(19) United States
(12) Patent Application Publication
(10) Pub. No.: US 2025/0066349 A1
(43) Pub. Date: Feb. 27, 2025

(54) CYCLIC COMPOUNDS AND THEIR USE FOR THE TREATMENT OF NEUROLOGICAL DISORDERS

(71) Applicant: Prothena Biosciences Limited, Dublin (IE)

(72) Inventor: Stéphane DE LOMBAERT, Chester, CT (US)

(21) Appl. No.: 18/717,600

(22) PCT Filed: Dec. 9, 2022
(86) PCT No.: PCT/US2022/052453

§ 371 Date: Jun. 7, 2024

Related U.S. Application Data
(60) Provisional application No. 63/288,154, filed on Dec. 10, 2021.
(60) Provisional application No. 63/288,161, filed on Dec. 10, 2021.
(60) Provisional application No. 63/288,167, filed on Dec. 10, 2021.
(60) Provisional application No. 63/288,197, filed on Dec. 10, 2021.

(51) Int. Cl. C07D 471/04; A61K 31/4184; A61K 31/427; A61K 31/428; A61K 31/4375; A61K 31/4439; A61K 31/444; A61K 31/454; A61K 31/4545; A61K 31/4741; A61K 31/4743; A61K 31/4745; A61K 31/496; A61K 31/4985; A61K 31/519; C07D 417/14; C07D 471/14; C07D 487/04; C07D 491/048; C07D 491/052; C07D 495/04; C07D 498/04; C07D 513/04; C07D 519/00

(52) U.S. Cl. CPC C07D 471/04; A61K 31/4184; A61K 31/427; A61K 31/428; A61K 31/4375; A61K 31/4439; A61K 31/444; A61K 31/454; A61K 31/4545; A61K 31/4741; A61K 31/4743; A61K 31/4745; A61K 31/496; A61K 31/4985; A61K 31/519; C07D 417/14; C07D 471/14; C07D 487/04; C07D 491/048; C07D 491/052; C07D 495/04; C07D 498/04; C07D 513/04; C07D 519/00

(57) ABSTRACT

This disclosure provides compounds of formula (I) and pharmaceutically acceptable salts thereof, that inhibit Dual specificity tyrosine-phosphorylation-regulated kinase 1A (DYRK1A). These chemical entities are useful, e.g., for treating a condition, disease or disorder in which increased (e.g., excessive) DYRK1A activation contributes to the pathology and/or symptoms and/or progression of the condition, disease or disorder (e.g., a neurological disorder) in a subject (e.g., a human). This disclosure also provides compositions containing the same as well as methods of using and making the same.

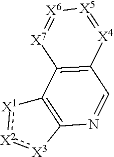

### TECHNICAL FIELD

This disclosure provides compounds and pharmaceutically acceptable salts thereof, that inhibit Dual specificity tyrosine-phosphorylation-regulated kinase 1A (DYRK1A). These compounds are useful, e.g., for treating a condition, disease or disorder in which increased (e.g., excessive) DYRK1A activation contributes to the pathology and/or symptoms and/or progression of the condition, disease or disorder (e.g., a neurological disorder in a subject (e.g., a human). This disclosure also provides compositions containing the same as well as methods of using and making the same.

### BACKGROUND

Dual-specificity tyrosine phosphorylation-regulated kinase 1A (DYRK1A) is a 763 amino acid, 85 kDa serine/threonine/tyrosine kinase located on chromosome 21 (2122.2). DYRK1A possesses catalytic activity that is regulated by autophosphorylation of a tyrosine residue (Y321) which results in constitutively active serine/threonine kinase activity. See Abbassi, et al., Pharmacology & Therapeutics, 151, 87-98 (2015). Since DYRK1A is constitutively active, its activity is dosage dependent. Thus, both elevated levels and depressed levels of DYRK1A, (relative to wild-type levels) have been shown to lead to neurological impairment. See Duchon and Herault, Front Behav. Neurosci. 10, 104-104 (2016). DYRK1A is also a member of a large family of CMGC kinases, which include cyclin-dependent kinases (CDKs), mitogen-activated protein kinases (MAPKs), glycogen synthase kinases (GSKs), and CDC-like kinases (CLKs).

DYRK1A additionally has been shown to have a role in cell cycle regulation, at least in part by phosphorylating (and thus inhibiting) the nuclear factor of activated T cells (NFAT) family of transcription factors. Additionally, over 20 substrates of DYRK1A have been identified, including cell signaling, chromatin modulation, gene expression, alternative splicing, cytoskeletal, and synaptic function. See Abassi, et al, (2016). DYRK1A dysregulation is implicated in various disease states such as Alzheimer's disease, autism, and Down syndrome. In some cases, novel mutations in DYRK1A have been associated with autism phenotypes. See e.g., Dang, et al., Molecular Psychiatry, 23, 747-758 (2018).

DYRK1A is also known to play an important role in brain development. For example, reduced DYRK1A activity (such has having a single copy of loss of function mutation) during neural development results in intellectual disability phenotypes. Conversely, trisomy 21 in Down syndrome individuals is associated with a triplication of the DYRK1A gene, which results in elevated DYRK1A activity. DYRK1A is located on chromosome 21, specifically within the “Down syndrome critical region” a portion of chromosome 21 that includes genes particularly relevant for developing Down syndrome phenotypes. As a result, individuals with Down syndrome have three copies of DYRK1A, and since DYRK1A is dosage sensitive, the elevated levels of DYRK1A in such individuals markedly affects the localization and function of the DYRK1A protein. The expression of DYRK1A is also elevated in the CNS in individuals with neurodegenerative diseases, such as Parkinson's disease, Pick's disease, and Alzheimer's disease.

Moreover, approximately 50% of individuals with Down syndrome ultimately develop Alzheimer's disease, with symptoms generally beginning between the ages of 40 and 60. DYRK1A phosphorylates amyloid precursor protein (APP) which promotes the production of pathogenic amyloid-D peptide (AD). Dyrk1A also phosphorylates tau both directly and indirectly (see Abassi, et al, (2016)). Both amyloid-O and tau pathologies are associated with Down syndrome phenotypes.

Normalization of DYRK1A gene dosage by crossing Ts65Dn mice (DS model) with DYRK1A knockout mice mice reverses many Alzheimer's-like phenotypes. See Garcia-Cerro et al., 2017. In individuals with Down Syndrome, DYRK1A mRNA levels, protein levels, and kinase activity are increased by ˜50%, reflecting the number of gene copies. See Liu et al., 2008; see also Wegiel et al., 2011.

Because no treatment is available for these neurological disorders, the prognosis for individuals with, for example, Alzheimer's disease is poor. This can be particularly devastating because Alzheimer's disease is responsible for a sharp decline in survival in individuals with Down syndrome that are over 45 years old. Only about 25% of those with Down syndrome live more than 60 years, and most of those have developed Alzheimer's disease.

Across all individuals, dementia remains a significant leading unmet medical need and a costly burden on public health. Currently, 1 in 3 seniors develops dementia, and about 70% of dementia cases are attributed to Alzheimer's disease. Some 11% of Americans over age 65 has AD, which constitutes over 6.2 million in 2021. This figure is projected to exceed 12 million in 2050 (www.Alz.org).

Presently, no therapies have been approved to treat Alzheimer's disease associated with Down syndrome, which represents a significant unmet medical need. Some DYRK1A inhibitors have been tested in vitro or in animal preclinical models to treat Alzheimer's disease or Down syndrome, however, since DYRK1A is a member of the highly conserved CMGC family of kinases, identifying compounds that selectively target DYRK1A has proved challenging. Thus, there remains a need to identify DYRK1A inhibitors to treat Down syndrome, Alzheimer's disease, Alzheimer's disease associated with Down syndrome, and other neurodegenerative and neurological diseases.

### SUMMARY

Some embodiments provide compounds of Formula (I):

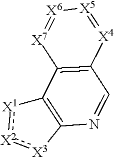

or a pharmaceutically acceptable salt thereof, wherein:
each dashed line represents a single bond or a double bond;
X¹ is CR^{1A}R^{1B}, NR^{A}, S, or O;
X² is CR^{2A}R^{2B}, C(═O), or NR^{B};
X³ is CR^{3A}R^{3B}, NR^{C}, S, or O;
X⁴ is CR⁴ or N;
X⁵ is CR⁵ or N;
X⁶ is CR⁶ or N;
X⁷ is CH, CF, or N;
R^{1A} is hydrogen, C1-C6 alkyl, —C(═O)NR^{F}R^{G}, —(C0-C6 alkyl)-5-6 membered 20 heteroaryl,
   —C(═O)-3-6 membered heterocyclyl optionally substituted with C1-C6 alkyl, a phenyl optionally substituted with halogen or —CO₂H, a 3-6 membered heterocyclyl optionally substituted with C1-C6 alkyl or —S(O₂)—C1-C6 alkyl, or a C3-C6 cycloalkyl optionally substituted with hydroxyl, —C(═O)NR^{F}R^{G}, —NR^{F}R^{G}, —CO₂R^{H}, or C1-C6 alkoxy;
R^{1B} is hydrogen or absent, wherein R^{1B} is absent when X [symbol] 2 is a double bond;
R^{2A} is hydrogen, C1-C6 alkyl, C1-C6 haloalkyl, C3-C6 cycloalkyl, 5-6 membered heteroaryl, a phenyl optionally substituted with —CO₂H, or a 3-6 membered heterocyclyl optionally substituted with C1-C6 alkyl, C1-C6 haloalkyl, C1-C6 hydroxyalkyl, or —(C0-C6 alkyl)-3-6 membered cycloalkyl optionally substituted with —CO₂H;
R^{2B} is hydrogen or absent, wherein R^{2B} is absent when either of x¹ [symbol] x² or x² [symbol] x³ is a double bond;
R^{3A} is hydrogen;
R^{3B} is hydrogen, C1-C6 alkyl, or absent, wherein R^{3B} is absent when x² [symbol] x³ is a double bond;
R⁴ is hydrogen, halogen, C1-C6 alkyl, or 3-6 membered heterocyclyl optionally substituted with C1-C6 alkyl;
R⁵ is hydrogen, —CO₂H, —C(═O)OCH₃, C1-C6 alkyl optionally substituted with hydroxyl, or a 5-6 membered heteroaryl optionally substituted with C1-C6 alkyl;
R⁶ is hydrogen or a 5-6 membered heteroaryl optionally substituted with 1-2 substituents independently selected from R^{D}, C1-C6 alkyl, and 4-6 membered heterocyclyl optionally substituted with hydroxyl;
R^{A} and R^{B} are independently absent, hydrogen, C1-C6 alkyl optionally substituted with 3-6 membered heterocyclyl or 5-6 membered heteroaryl, or a C3-C6 cycloalkyl optionally substituted with hydroxyl or C1-C6 alkoxy;
R^{C} is absent, hydrogen, or methyl;
R^{D} is

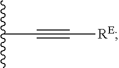

R^{E} is a C1-C6 alkyl, phenyl, 5-6 membered heteroaryl, or 4-6 membered heterocyclyl; wherein the C1-C6 alkyl, phenyl, 5-6 membered heteroaryl, and 4-6 membered heterocyclyl are each optionally substituted with 1-2 independently selected R^{I};
each R^{F} and R^{G} are independently selected from hydrogen and C1-C6 alkyl; or R^{F} and R^{G} together with the nitrogen atom to which they are attached form a 4-6 membered heterocyclyl optionally substituted with C1-C6 alkyl;
R^{H} is hydrogen or C1-C6 alkyl;
each R^{I} is independently C1-C6 alkyl, C1-C6 alkoxy, halogen, hydroxyl, cyano, and trifluoromethyl.

Some embodiments provide a compound of Formula (II):

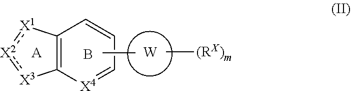

or a pharmaceutically acceptable salt thereof, wherein:
each dashed line represents a single bond or a double bond;
Rings A and B are aromatic;
Ring W is a 9 membered heteroaryl, a 9 membered heterocyclyl, or a 9 membered cycloalkyl;
each R^{X} is independently selected from C1-C6 alkyl, 3-6 membered heterocyclyl optionally substituted with C1-C6 alkyl, and C3-C6 cycloalkyl;
X¹ is CH, S, N, or NR^{A};
X² is N, CH, or CR²;
X³ is N, NR^{B}, O, CR³, or CH;
X⁴ is CH or N;
R² is benzyl or

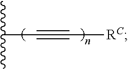

R³ is C1-C6 alkyl;
R^{A} is C1-C6 alkyl or C3-C6 cycloalkyl;
R^{B} is hydrogen or C3-C6 cycloalkyl;
R^{C} is 3-6 membered heterocyclyl optionally substituted with C1-C6 alkyl;
m is 0, 1, or 2; and
n is 0 or 1.

Some embodiments provide a compound of Formula (III):

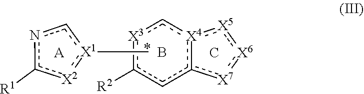

or a pharmaceutically acceptable salt thereof, wherein:
each dashed line represents a single bond or a double bond;
Rings A and B are aromatic;
Ring C is aromatic or partially saturated;
X¹ is C or N;
X² is CH, O, or S;
X³ is CH or N;
X⁴ is C or N;
X⁵ is CH, C(═O), O, S, NH, or NR^{A};
X⁶ is CH, N, or O;
X⁷ is CH, CR⁷, CH₂, CR^{B}R^{C}, or N;
R¹ is hydrogen or —XR^{D};
R² is hydrogen, —NHC(═O)-3-6 membered heterocyclyl optionally substituted with C1-C6 haloalkyl, or joins with the bond denoted with *;
R⁷ is C1-C6 alkyl optionally substituted with a 3-6 membered heterocyclyl optionally substituted with C(═O)OH;
R^{A} is -(3-6 membered heterocyclyl)ₘ-(C1-C6 alkyl)ₙ;
R^{B} and R^{C} are independently hydrogen or C1-C6 alkyl;
X is ethynylene, —NHC(═O)—, or —NHC(═O)OCH₂—;
R^{D} is 3-6 membered heterocyclyl optionally substituted with C1-C6 alkyl, 5-6 membered heteroaryl optionally substituted with C1-C6 alkyl, or C6-C10 aryl optionally substituted with C1-C6 alkyl;
m is 0 or 1; and
n is 0, 1, or 2;
provided that when n is 2, m is 1.

Some embodiments provide a compound of Formula (IV):

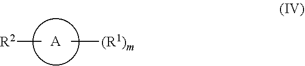

or a pharmaceutically acceptable salt thereof, wherein:
Ring A is a 12-14 membered fused tricyclic heterocyclyl comprising 2-5 nitrogen atoms or a 12-14 membered fused tricyclic heteroaryl comprising 2-5 nitrogen atoms;
R¹ is cyano, C1-C6 alkyl, —NHC(═O)(C1-C6 alkylene)ₙR^{A}, -Q-phenyl optionally substituted with 1-2 substituents independently selected from halogen, hydroxyl, and C1-C6 alkoxy; C3-C6 cycloalkyl optionally substituted with hydroxyl, —(C1-C6 alkylene)ₚ-5-10 membered heteroaryl, or 5-10 membered heterocyclyl;
R² is hydrogen, cyano, C1-C6 alkyl, —C(═O)—C1-C6 alkyl, —(SO₂)C1-C6 alkyl, —CO₂R^{B}, C1-C6 alkoxy optionally substituted with —NR^{C}R^{D};
R^{A} is 4-6 membered heterocyclyl optionally substituted with C1-C6 alkyl, or 5-10 membered heteroaryl optionally substituted with C1-C6 alkoxy or C1-C6 alkyl;
R^{B}, R^{C}, and R^{D} are independently hydrogen or C1-C6 alkyl;
Q is a bond or O;
m is 0 or 1;
n is 0 or 1; and
p is 0 or 1.

Also provided herein is a pharmaceutical composition comprising a compound of Formula (I), (II), (III), or (IV), or a pharmaceutically acceptable salt of any of the foregoing, and a pharmaceutically acceptable carrier.

Provided herein is a method for treating a neurological disorder in a subject in need thereof, the method comprising administering to the subject a therapeutically effective amount of a compound of Formula (I), (II), (III), or (IV), or a pharmaceutically acceptable salt of any of the foregoing, or a pharmaceutical composition as provided herein.

Also provided herein is a method for treating a neurological disorder in a subject in need thereof, the method comprising (a) determining that the neurological disorder is associated with a dysregulation of a DYRK1A gene, a DYRK1A protein, or expression or activity or level of any of the same; and (b) administering to the subject a therapeutically effective amount of a compound of Formula (I), (II), (III), or (IV), or a pharmaceutically acceptable salt of any of the foregoing, or a pharmaceutical composition as provided herein.

Provided herein is a method of treating a DYRK1A-associated disease or disorder in a subject, the method comprising administering to a subject identified or diagnosed as having a DYRK1A-associated disease or disorder a therapeutically effective amount of a compound of Formula (I), (II), (III), or (IV), or a pharmaceutically acceptable salt of any of the foregoing, or a pharmaceutical composition as provided herein.

This disclosure also provides a method of treating a DYRK1A-associated neurological disorder in a subject, the method comprising: determining that the neurological disorder in the subject is a DYRK1A-associated disease or disorder; and administering to the subject a therapeutically effective amount of a compound of Formula (I), (II), (III), or (IV), or a pharmaceutically acceptable salt of any of the foregoing, or a pharmaceutical composition as provided herein.

Further provided herein is a method of treating a DYRK1A-associated neurological disorder in a subject, the method comprising administering to a subject identified or diagnosed as having a DYRK1A-associated neurological disorder a therapeutically effective amount of a compound of Formula (I), (II), (III), or (IV), or a pharmaceutically acceptable salt of any of the foregoing, or a pharmaceutical composition as provided herein.

This disclosure also provides a method of treating a DYRK1A-associated neurological disorder in a subject, the method comprising: determining that the neurological disorder in the subject is a DYRK1A-associated neurological disorder; and administering to the subject a therapeutically effective amount of a compound of Formula (I), (II), (III), or (IV), or a pharmaceutically acceptable salt of any of the foregoing, or a pharmaceutical composition as provided herein.

Provided herein is a method of treating a subject, the method comprising administering a therapeutically effective amount of a compound of Formula (I), (II), (III), or (IV), or a pharmaceutically acceptable salt of any of the foregoing, or a pharmaceutical composition as provided herein, to a subject having a clinical record that indicates that the subject has a dysregulation of a DYRK1A gene, a DYRK1A protein, or expression or activity or level of any of the same.

This disclosure also provides a method for inhibiting DYRK1A in a mammalian cell, the method comprising contacting the mammalian cell with a therapeutically effective amount of a compound of Formula (I), (II), (III), or (IV), or a pharmaceutically acceptable salt of any of the foregoing salt of any of the foregoing.

The details of one or more embodiments of this disclosure are set forth in the accompanying drawings and the description below. Other features and advantages of the present disclosure will be apparent from the description and the claims.

### DETAILED DESCRIPTION

Dual-specificity tyrosine phosphorylation-regulated kinase TA (DYRK1A) is a member of the dual-specificity tyrosine phosphorylation regulated kinase (DYRK) family, which is also part of the larger CGMC family of kinases. DYRK1A is a 763 amino acid, 85 kDa serine/threonine kinase located on chromosome 21. DYRK1A contains a nuclear targeting signal sequence, a protein kinase domain, a leucine zipper motif, and a highly conservative 13-consecutive-histidine repeat. Alternative splicing DYRK1A generates several transcript variants differing from each other either in either the 5′ untranslated region or in the 3′ coding region resulting in at least five different isoforms.

DYRK1A possesses catalytic activity that is regulated by autophosphorylation of a tyrosine residue (Y321) which results in constitutively active serine/threonine kinase activity. Since DYRK1A is constitutively active, its activity is dosage dependent. Thus, both elevated levels and depressed levels of DYRK1A (relative to wild-type levels) have been shown to lead to neurological impairment.

DYRK1A displays a broad substrate spectrum (e.g., broad range of targets) including splicing factors, synaptic proteins, and transcription factors. It is ubiquitously expressed in all mammalian tissues and cells, although at different levels, with particularly high levels in embryonic and adult brain tissues. The human DYRK1A gene is a candidate gene to treat several Down syndrome characteristics, including intellectual impairment and Alzheimer's disease associated with Down syndrome, due to its localization in the Down syndrome critical region on chromosome 21 and its role in brain function. Notably, Drosophila with deleterious mutations in the ortholog of DYRK1A (“Minibrain”) have a reduced number of neurons in their central nervous system. Likewise, mice heterozygous for a disrupted allele of the Dyrk1a gene exhibit decreased viability, behavioral alterations, and delayed growth. Fotaki, et al., Mol Cell Biol., 22(18): 6636-6647 (2014).

The identification of hundreds of genes deregulated by DYRK1A overexpression and numerous cytosolic, cytoskeletal and nuclear proteins, including transcription factors, phosphorylated by DYRK1A, indicates that DYRK1A overexpression is central for the deregulation of multiple pathways in the developing and aging brain of individuals with Down syndrome. Identifying DYRK1A cell signaling or transduction pathways can lead to a better understanding of how DYRK1A overexpression (or under expression) leads to the various disease states in which it is known to be involved. Specifically, DYRK1A is known to be active in activated PI3K/Akt signaling, a pathway largely involved in neuronal development, growth, and survival. DYRK1A is also known to be active in ASK1/JNK1 activity and inhibitors of DYRK1A may induce neuronal death and apoptosis. DYRK1A is also known to phosphorylate p53 during embryonic brain development, and inhibitors of DYRK1A can prevent neuronal proliferation alteration. DYRK1A also phosphorylates synaptic proteins Amph 1, Dynamin 1, and Synaptojanin, which are involved in the regulation of endocytosis and inhibitors of DYRK1A can retain synaptic plasticity through preventing alteration of the number, size, and morphology of dendritic spines. DYRK1A also phosphorylates inhibit presenilin 1, the catalytic sub-unit of γ-secretase. Ryu, et al., J Neurochem., 115(3): 574-84 (2010).

DYRK1A overexpression leads to structural and functional alterations including intellectual disability and dementia, e.g., Alzheimer's disease. In particular, genes involved in learning disorders, synaptic flexibility changes, memory loss, and abnormal cell cycles, result in neuropathological symptoms similar to dementia associated with Alzheimer's disease. DYRK1A can also affect the proliferation and differentiation of neuronal progenitors, thus influencing neurogenesis and brain growth. It can also affect neurotransmission and dendritic spine formation through its interaction with synaptic proteins and the cytoskeleton.

One potential source of treatment are inhibitors of DYRK1A. Inhibitors that can normalize DYRK1A levels in Down syndrome may improve synaptic plasticity and delay the onset of Alzheimer's disease pathology, including tau hyperphosphorylation. Therefore, inhibiting DYRK1A activity in individuals with Down syndrome might counteract the phenotypic effects of its overexpression and is a potential avenue for the treatment of such developmental defects and prevention and/or mitigation of age-associated neurodegeneration, including Alzheimer's disease associated with Down syndrome. Studies have shown that inhibition of overexpressed DYRK1A resulted in normal DYRK1A levels and been found to improve cognitive and behavioral deficits in transgenic models. See, e.g., Stringer, et al., Mol Genet Genomic Med, 5, 451-465 (2017) and Feki and Hibaoui, Brain Sci, 8, 187 (2018). However, despite promising results there is considerable variation across studies in terms of outcomes. Discrepancies were attributed to differences in model, dose, route of administration, the composition of the inhibitor, and timing of administration.

Epigallocatechin gallate (EGCG) is the primary flavonoid of green tea and has been investigated for its therapeutic effects, which include anti-oxidative, anti-inflammatory, anti-cancer, anti-infective and neuroprotective activity. See, Bhat, et al. Towards the discovery of drug-like epigallocatechin gallate analogs as Hsp90 inhibitors, Bioorg Med Chem Lett, 24, 2263-2266 (2014). EGCG is a non-ATP competitive DYRK1A inhibitor and studies have shown that green tea extract comprising 41% EGCG were able to alleviate cognitive decline seen in transgenic mice over expressing DYRK1A. ECGC has also been shown to improve memory recognition and working memory. However, ECGC is not significantly selective and has numerous off-target effects, thus reducing its potential long-term use.

SM07883 is an orally bioavailable (% F 92% in mice, 109% in monkey), BBB penetrant, DYRK1A inhibitor (IC50 1.6 nM) that also shows potent inhibition for DYRK1B, CLK4, and GSK3β in kinase assays. It was found to protect against tau hyperphosphorylation in mouse models. SM07883 was tested for treatment of Alzheimer's disease in a phase 1 study in Australia (ACTRN12619000327189). However, according to the study description page at www.anzctr.org.au, the date of last data collection was in May 2019 and no results have been published for the trial.

This disclosure provides compounds of Formula (I), (II), (III), and (VI), and pharmaceutically acceptable salts of any of the foregoing, that inhibit Dual specificity tyrosine-phosphorylation-regulated kinase 1A (DYRK1A). These chemical entities are useful, e.g., for treating a condition, disease or disorder in which increased (e.g., excessive) DYRK1A activation contributes to the pathology and/or symptoms and/or progression of the condition, disease or disorder (e.g., a neurological disorder in a subject (e.g., a human). This disclosure also provides compositions containing the same as well as methods of using and making the same.

### Definitions

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entireties. In case of conflict, the present specification, including definitions, will control.

The term “compound,” as used herein is meant to include all stereoisomers, geometric isomers, tautomers, and isotopically enriched variants of the structures depicted. Compounds herein identified by name or structure as one particular tautomeric form are intended to include other tautomeric forms unless otherwise specified. For example, if quinazolin-4-ol is encompassed by a claim or embodiment, then quinazolin-4(3H)-one is also covered by the claim or embodiment (see below).

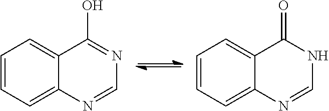 [SMILES: Oc1ncnc2ccccc12 ; O=c1[nH]cnc2ccccc12]

It will be appreciated that certain compounds provided herein may contain one or more centers of asymmetry and may therefore be prepared and isolated in a mixture of isomers such as a racemic mixture, or in an enantiomerically pure form. Unless otherwise indicated, when a disclosed compound is named or depicted by a structure without specifying the stereochemistry (e.g., a “flat” structure) and has one or more chiral centers, it is understood to represent all possible stereoisomers of the compound.

The term “about” when referring to a number or a numerical range means that the number or numerical range referred to is an approximation, for example, within experimental variability and/or statistical experimental error, and thus the number or numerical range may vary up to ±10% of the stated number or numerical range.

The term “acceptable” with respect to a formulation, composition or ingredient, as used herein, means having no persistent detrimental effect on the general health of the subject being treated.

The term “inhibit” or “inhibition of” means to reduce by a measurable amount, or to prevent entirely (e.g., 100% inhibition).

The term “therapeutically effective amount,” as used herein, refer to a sufficient amount of a chemical entity being administered which will relieve to some extent one or more of the symptoms of the disease or condition being treated. The result includes reduction and/or alleviation of the signs, symptoms, or causes of a disease, or any other desired alteration of a biological system. For example, a therapeutically effective amount” for therapeutic uses is the amount of the composition comprising a compound as disclosed herein required to provide a clinically significant decrease in disease symptoms. An appropriate “therapeutically effective” amount in any individual case is determined using any suitable technique, such as a dose escalation study.

The term “excipient” or “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described herein form with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and omithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to: rectal, oral, intravenous, aerosol, parenteral, ophthalmic, pulmonary, and topical administration.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The term “halogen” refers to fluoro (F), chloro (Cl), bromo (Br), or iodo (I).

The term “oxo” refers to a divalent doubly bonded oxygen atom (i.e., “═O”). As used herein, oxo groups are attached to carbon atoms to form carbonyls.

The term “hydroxyl” refers to an —OH radical.

The term “cyano” refers to a —CN radical.

The term “alkyl” refers to a saturated acyclic hydrocarbon radical that can be straight chain or branched chain, containing the indicated number of carbon atoms. For example, C1-C10 indicates that the group may have from 1 to 10 (inclusive) carbon atoms in it. Alkyl groups can either be unsubstituted or substituted with one or more substituents. Non-limiting examples include methyl, ethyl, iso-propyl, tert-butyl, n-hexyl. The term “saturated” as used in this context means only single bonds present between constituent carbon atoms and other available valences occupied by hydrogen and/or other substituents as defined herein. A “C0” alkyl refers to a bond, e.g., phenyl-(C0 alkyl)-OH corresponds to phenol.

The term “haloalkyl” refers to an alkyl group in which one or more hydrogen atoms is/are replaced with an independently selected halogen.

The term “alkoxy” refers to an —O-alkyl radical (e.g., —OCH₃).

The term “aryl” refers to a 6-20 carbon atom monocyclic, bicyclic, or tricyclic group wherein at least one ring in the system is aromatic. Examples of aryl groups include phenyl, naphthyl, tetrahydronaphthyl, and the like.

The term “cycloalkyl” as used herein refers to cyclic hydrocarbon groups having the indicated number of carbon atoms, e.g., 3 to 20 ring carbons (C3-C20), 3 to 16 ring carbons (C3-C16), 3-10 ring carbons (C3-C10), or 3-6 ring carbons (C3-C6). Cycloalkyl groups are saturated or partially unsatured (but not aromatic). Examples of cycloalkyl groups include, without limitation, cyclopropyl, cyclobutyl, cyclopentyl, cyclopentenyl, cyclohexyl, cyclohexenyl, cycloheptyl, and cyclooctyl. Cycloalkyl may include multiple fused and/or bridged rings. Non-limiting examples of fused/bridged cycloalkyl includes: bicyclo[1.1.0]butane, bicyclo[2.1.0]pentane, bicyclo[1.1.1]pentane, bicyclo[3.1.0]hexane, bicyclo[2.1.1]hexane, bicyclo[3.2.0]heptane, bicyclo[4.1.0]heptane, bicyclo[2.2.1]heptane, bicyclo[3.1.1]heptane, bicyclo[4.2.0]octane, bicyclo[3.2.1]octane, bicyclo[2.2.2]octane, and the like. Cycloalkyl also includes spirocyclic rings (e.g., spirocyclic bicycle wherein two rings are connected through just one atom). Non-limiting examples of spirocyclic cycloalkyls include spiro[2.2]pentane, spiro[2.5]octane, spiro[3.5]nonane, spiro[3.5]nonane, spiro[3.5]nonane, spiro[4.4]nonane, spiro[2.6]nonane, spiro[4.5]decane, spiro[3.6]decane, spiro[5.5]undecane, and the like.

The term “heteroaryl”, as used herein, means a monocyclic, bicyclic, or tricyclic group having 5 to 20 ring atoms (5-20 membered heteroaryl), such as 5, 6, 9, 10, or 14 ring atoms; wherein at least one ring in the system contains one or more heteroatoms independently selected from the group consisting of N, O, and S and at least one ring in the system is aromatic (but does not have to be a ring which contains a heteroatom, e.g. tetrahydroisoquinolinyl, e.g., tetrahydroquinolinyl). Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others. In some embodiments, the heteroaryl is selected from thienyl, pyridinyl, furyl, pyrazolyl, imidazolyl, isoindolinyl, pyranyl, pyrazinyl, and pyrimidinyl. For purposes of clarification, heteroaryl also includes aromatic lactams, aromatic cyclic ureas, or vinylogous analogs thereof, in which each ring nitrogen adjacent to a carbonyl is tertiary (i.e., all three valences are occupied by non-hydrogen substituents), such as one or more of pyridone (e.g.,

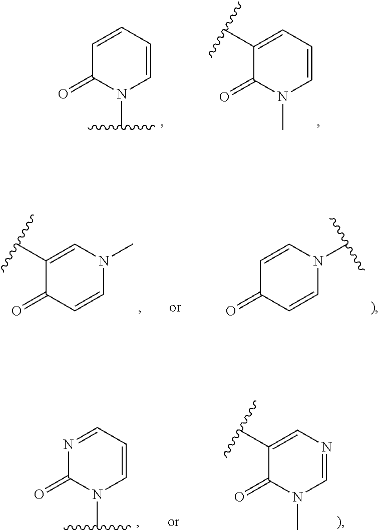

pyrimidone (e.g.,

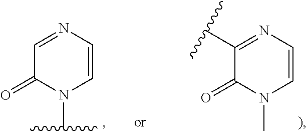

pyridazinone (e.g.,

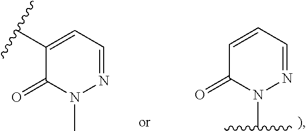

pyrazinone

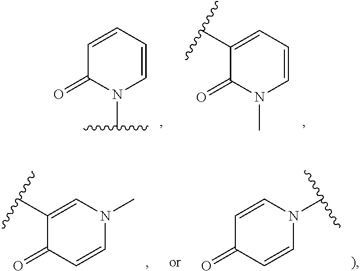

and imidazolone (e.g.

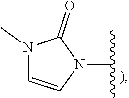 [SMILES: *n1ccn(C)c1=O]

wherein each ring nitrogen adjacent to a carbonyl is tertiary (i.e., the oxo group (i.e., “═O”) herein is a constituent part of the heteroaryl ring).

The term “heterocyclyl” refers to monocyclic, bicyclic, or tricyclic saturated or partially unsaturated ring systems with 3-16 ring atoms (e.g., 5-8 membered monocyclic, 8-12 membered bicyclic, or 11-14 membered tricyclic ring systems) having 1-3 heteroatoms if monocyclic, 1-6 heteroatoms if bicyclic, or 1-9 heteroatoms if tricyclic. The heteroatoms are selected from the group consisting of O, N, or S (e.g., carbon atoms and 1-3, 1-6, or 1-9 heteroatoms of N, O, or S if monocyclic, bicyclic, or tricyclic, respectively), wherein one or more ring atoms may be substituted by 1-3 oxo (forming, e.g., a lactam) and one or more N or S atoms may be substituted by 1-2 oxido (forming, e.g., an N-oxide, an S-oxide, or an S,S-dioxide), valence permitting; and wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, tetrahydropyridyl, dihydropyrazinyl, dihydropyridyl, dihydropyrrolyl, dihydrofuranyl, dihydrothiophenyl, and the like. Heterocyclyl may include multiple fused and bridged rings. Non-limiting examples of fused/bridged heteorocyclyl includes: 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, 3-azabicyclo[3.2.1]octane, 2-oxabicyclo[1.1.0]butane, 2-oxabicyclo[2.1.0]pentane, 2-oxabicyclo[1.1.1]pentane, 3-oxabicyclo[3.1.0]hexane, 5-oxabicyclo[2.1.1]hexane, 3-oxabicyclo[3.2.0]heptane, 3-oxabicyclo[4.1.0]heptane, 7-oxabicyclo[2.2.1]heptane, 6-oxabicyclo[3.1.1]heptane, 7-oxabicyclo[4.2.0]octane, 2-oxabicyclo[2.2.2]octane, 3-oxabicyclo[3.2.1]octane, and the like. Heterocyclyl also includes spirocyclic rings (e.g., spirocyclic bicycle wherein two rings are connected through just one atom). Non-limiting examples of spirocyclic heterocyclyls include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 7-azaspiro[4.5]decane 2,5-diazaspiro[3.6]decane, 3-azaspiro[5.5]undecane, 2-oxaspiro[2.2]pentane, 4-oxaspiro[2.5]octane, 1-oxaspiro[3.5]nonane, 2-oxaspiro[3.5]nonane, 7-oxaspiro[3.5]nonane, 2-oxaspiro[4.4]nonane, 6-oxaspiro[2.6]nonane, 1,7-dioxaspiro[4.5]decane, 2,5-dioxaspiro[3.6]decane, 1-oxaspiro[5.5]undecane, 3-oxaspiro[5.5]undecane, 3-oxa-9-azaspiro[5.5]undecane and the like.

As used herein, examples of aromatic rings include: benzene, pyridine, pyrimidine, pyrazine, pyridazine, pyridone, pyrrole, pyrazole, oxazole, thioazole, isoxazole, isothiazole, and the like.

The term “saturated” as used in this context means only single bonds present between constituent atoms.

As used herein, when a ring is described as being “partially unsaturated”, it means said ring has one or more additional degrees of unsaturation (in addition to the degree of unsaturation attributed to the ring itself; e.g., one or more double or triple bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

For the avoidance of doubt, and unless otherwise specified, for rings and cyclic groups (e.g., aryl, heteroaryl, heterocyclyl, cycloalkyl, and the like described herein) containing a sufficient number of ring atoms to form bicyclic or higher order ring systems (e.g., tricyclic ring systems), it is understood that such rings and cyclic groups encompass those having fused rings, including those in which the points of fusion are located (i) on adjacent ring atoms (e.g., [x.x.0] ring systems, in which 0 represents a zero atom bridge (e.g., N ))

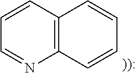

(ii) a single ring atom (spiro-fused ring systems) (e.g.,

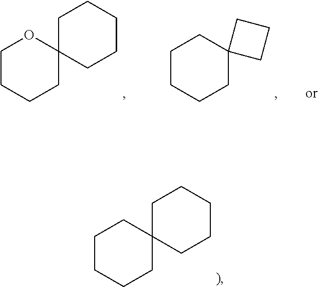 [SMILES: C1CCC2(CC1)CCCCO2 ; C1CCC2(CC1)CCC2 ; C1CCC2(CC1)CCCCC2]

or (iii) a contiguous array of ring atoms (bridged ring systems having all bridge lengths >0) (e.g.,

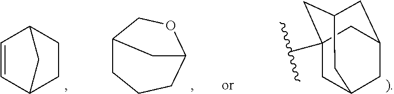 [SMILES: C1=CC2CCC1C2 ; C1CC2COC(C1)C2 ; *C12CC3CC(CC(C3)C1)C2]

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In addition, the compounds generically or specifically disclosed herein are intended to include all tautomeric forms. Thus, by way of example, a compound containing the moiety:

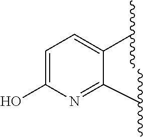 [SMILES: *c1ccc(O)nc1*]

encompasses the tautomeric form containing the moiety:

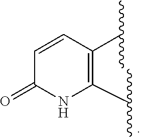 [SMILES: *c1ccc(=O)[nH]c1*]

Similarly a pyridinyl or pyrimidinyl moiety that is described to be optionally substituted with hydroxyl encompasses pyridone or pyrimidone tautomeric forms.

Dashed lines in chemical structures, for example, x¹ [symbol] x² and x² [symbol] x³ represent single or double bonds. One skilled in the art understands that, in this structure,

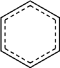

for example, the maximum number of double bonds is three.

### Compounds of Formula (I)

The substituent groups used in this section (e.g., R¹, R², and the like) refer solely to the groups in Formula (I).

Some embodiments provide a compound of Formula (1-0):

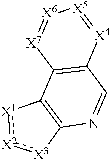

or a pharmaceutically acceptable salt thereof, wherein:
each dashed line represents a single bond or a double bond;
X¹ is CR^{1A}R^{1B}, NR^{A}, S, or O;
X² is CR^{2A}R^{2B}, C(═O), or NR^{B}.
X³ is CR^{3A}R^{3B}, NR^{C}, S, or O;
X⁴ is CR⁴ or N;
X⁵ is CR³ or N;
X⁶ is CR⁶ or N;
X⁷ is CH, CF, or N;
R^{1A} is hydrogen, C1-C6 alkyl, —C(═O)NR^{F}R^{G}, —(C0-C6 alkyl)-5-6 membered heteroaryl,
   —C(═O)-3-6 membered heterocyclyl optionally substituted with C1-C6 alkyl, a phenyl optionally substituted with halogen or —CO₂H, a 3-6 membered heterocyclyl optionally substituted with C1-C6 alkyl or —S(O₂)—C1-C6 alkyl, or a C3-C6 cycloalkyl optionally substituted with hydroxyl,
   —C(═O)NR^{F}R^{G}, —NR^{F}R^{G}, —CO₂R^{H}, or C1-C6 alkoxy;
R^{1B} is hydrogen or absent, wherein R^{1B} is absent when x¹ [symbol] x² is a double bond;
R^{2A} is hydrogen, halogen, C1-C6 alkyl, C1-C6 haloalkyl, C1-C6 alkoxy, C1-C6 haloalkoxy, C3-C6 cycloalkyl, 5-6 membered heteroaryl, a phenyl optionally substituted with halogen or —CO₂H, or a 3-6 membered heterocyclyl optionally substituted with C1-C6 alkyl, C1-C6 haloalkyl, C1-C6 hydroxyalkyl, or −(C0-C6 alkyl)-3-6 membered cycloalkyl optionally substituted with —CO₂H;
R^{2B} is hydrogen or absent, wherein R^{2B} is absent when either of x¹ [symbol] x² or x² [symbol] x³ is a double bond;
R^{3A} is hydrogen, cyano, or halogen;
R^{3B} is hydrogen, halogen, C1-C6 alkyl, C1-C6 haloalkyl, C1-C6 alkoxy, C1-C6 haloalkoxy, or absent, wherein R^{3B} is absent when x² [symbol] x³ is a double bond;
R⁴ is hydrogen, halogen, cyano, hydroxyl, C1-C6 alkyl, or 3-6 membered heterocyclyl optionally substituted with C1-C6 alkyl;
R⁵ is hydrogen, —CO₂H, —C(═O)OCH₃, C1-C6 alkyl optionally substituted with hydroxyl; C1-C6 haloalkyl, C1-C6 alkoxy, C1-C6 haloalkoxy, or a 5-6 membered heteroaryl optionally substituted with C1-C6 alkyl;
R⁶ is hydrogen or a 5-6 membered heteroaryl optionally substituted with 1-2 substituents independently selected from R^{D}, C1-C6 alkyl, and 4-6 membered heterocyclyl optionally substituted with hydroxyl;
R^{A} and R^{B} are independently absent, hydrogen, C1-C6 alkyl optionally substituted with 3-6 membered heterocyclyl or 5-6 membered heteroaryl, or a C3-C6 cycloalkyl optionally substituted with hydroxyl or C1-C6 alkoxy;
R^{C} is absent, hydrogen, or methyl;
R^{D} is

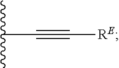

R^{E} is a C1-C6 alkyl, phenyl, 5-6 membered heteroaryl, or 4-6 membered heterocyclyl; wherein the C1-C6 alkyl, phenyl, 5-6 membered heteroaryl, and 4-6 membered heterocyclyl are each optionally substituted with 1-2 independently selected R^{I};
each R^{F} and R^{G} are independently selected from hydrogen and C1-C6 alkyl; or R^{F} and R^{G} together with the nitrogen atom to which they are attached form a 4-6 membered heterocyclyl optionally substituted with C1-C6 alkyl;
R^{H} is hydrogen or C1-C6 alkyl;
each R^{I} is independently C1-C6 alkyl, C1-C6 alkoxy, halogen, hydroxyl, cyano, or trifluoromethyl.

Some embodiments provide a compound of Formula (I):

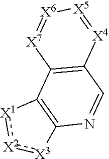

or a pharmaceutically acceptable salt thereof, wherein:
each dashed line represents a single bond or a double bond;
X¹ is CR^{1A}R^{1B}, NR^{A}, S, or O;
X² is CR^{2A}R^{2B}, C(═O), or NR^{B};
X³ is CR^{3A}R^{3B}, NR^{C}, S, or O;
X⁴ is CR⁴ or N;
X⁵ is CR⁵ or N;
X⁶ is CR⁶ or N;
X⁷ is CH, CF, or N;
R^{1A} is hydrogen, C1-C6 alkyl, —C(═O)NR^{F}R^{G}, —(C0-C6 alkyl)-5-6 membered heteroaryl,
   —C(═O)-3-6 membered heterocyclyl optionally substituted with C1-C6 alkyl, a phenyl optionally substituted with halogen or —CO₂H, a 3-6 membered heterocyclyl optionally substituted with C1-C6 alkyl or —S(O₂)—C1-C6 alkyl, or a C3-C6 cycloalkyl optionally substituted with hydroxyl,
   —C(═O)NR^{F}R^{G}, —NR^{F}R^{G}, —CO₂R^{H}, or C1-C6 alkoxy;
R^{1B} is hydrogen or absent, wherein R^{1B} is absent when x¹ [symbol] x² is a double bond;
R^{2A} is hydrogen, C1-C6 alkyl, C1-C6 haloalkyl, C3-C6 cycloalkyl, 5-6 membered heteroaryl, a phenyl optionally substituted with —CO₂H, or a 3-6 membered heterocyclyl optionally substituted with C1-C6 alkyl, C1-C6 haloalkyl, C1-C6 hydroxyalkyl, or —(C0-C6 alkyl)-3-6 membered cycloalkyl optionally substituted with —CO₂H;
R^{2B} is hydrogen or absent, wherein R^{2B} is absent when either of x¹ [symbol] x² or x² [symbol] x³ is a double bond;
R^{3A} is hydrogen;
R^{3B} is hydrogen, C1-C6 alkyl, or absent, wherein R^{3B} is absent when x² [symbol] x³ is a double bond;
R⁴ is hydrogen, halogen, C1-C6 alkyl, or 3-6 membered heterocyclyl optionally substituted with C1-C6 alkyl;
R⁵ is hydrogen, —CO₂H, —C(═O)OCH₃, C1-C6 alkyl optionally substituted with hydroxyl, or a 5-6 membered heteroaryl optionally substituted with C1-C6 alkyl;
R⁶ is hydrogen or a 5-6 membered heteroaryl optionally substituted with 1-2 substituents independently selected from R^{D}, C1-C6 alkyl, and 4-6 membered heterocyclyl optionally substituted with hydroxyl;
R^{A} and R^{B} are independently absent, hydrogen, C1-C6 alkyl optionally substituted with 3-6 membered heterocyclyl or 5-6 membered heteroaryl, or a C3-C6 cycloalkyl optionally substituted with hydroxyl or C1-C6 alkoxy;
R^{C} is absent, hydrogen, or methyl;
R^{D} is

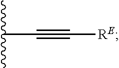

R^{E} is a C1-C6 alkyl, phenyl, 5-6 membered heteroaryl, or 4-6 membered heterocyclyl; wherein the C1-C6 alkyl, phenyl, 5-6 membered heteroaryl, and 4-6 membered heterocyclyl are each optionally substituted with 1-2 independently selected R^{I};
each R^{F} and R^{G} are independently selected from hydrogen and C1-C6 alkyl; or R^{F} and R^{G} together with the nitrogen atom to which they are attached form a 4-6 membered heterocyclyl optionally substituted with C1-C6 alkyl;
R^{H} is hydrogen or C1-C6 alkyl;
each R^{I} is independently C1-C6 alkyl, C1-C6 alkoxy, halogen, hydroxyl, cyano, or trifluoromethyl.

In some embodiments, X¹ is CR^{1A}R^{1B}, X² is CR^{2A}R^{2B}, X³ is NR^{C}, x¹ [symbol] x² is a double bond, x² [symbol] x³ is a single bond, R^{1B} is absent, and R^{2B} is absent.

In some embodiments, X¹ is NR^{A}, X² is CR^{2A}R²B, X³ is NR^{C}, x¹ [symbol] x² is a single bond, x² [symbol] x³ is a double bond, and R^{2B} and R^{C} are absent.

In some embodiments, X¹ is NR^{A}, X² is CR^{2A}R²B, X³ is NR^{C}, x¹ [symbol] x² is a double bond, x² [symbol] x³ is a single bond, and R^{2B} and R^{A} is absent.

In some embodiments, X¹ is S, X² is CR^{2A}R²B, X³ is CR^{3A}R^{3B} x¹ [symbol] x² is a single bond, x² [symbol] x³ is a double bond, R^{2B} is absent, and R^{3B} is absent.

In some embodiments, X¹ is S, X² is CR^{2A}R²B, X³ is NR^{C}, x¹ [symbol] x² is a single bond, x² [symbol] x³ is a double bond, and R^{2B} is absent.

In some embodiments, X¹ is NR^{A}, X² is CR^{2A}R²B, X³ is NR^{C}, x¹ [symbol] x² is a single bond, x² [symbol] x³ is a double bond, and R^{2B} and R^{C} are absent.

In some embodiments, X¹ is CR^{1A}R^{1B}, X² is NR^{B}, X³ is NR^{C}, x¹ [symbol] x² is a single bond, and x² [symbol] x³ is a single bond, and R^{B} is absent.

In some embodiments, X¹ is CR^{1A}R^{1B}, X² is C═O, X³ is NR^{C}, x¹ [symbol] x² is a single bond, x² [symbol] x³ is a single bond.

In some embodiments, X¹ is CR^{1A}R^{1B}, X² is CR^{2A}R^{2B}, X³ is O, x¹ [symbol] x² is a single bond, x² [symbol] x³ is a single bond.

In some embodiments, X¹ is CR^{1A}R^{1B}, X² is CR^{2A}R^{2B}, X³ is O, x¹ [symbol] x² is a double bond, x² [symbol] x³ is single bond, R^{1B} is absent, and R² is absent.

In some embodiments, the ring that includes X¹, X², and X³, contains one or two heteroatoms. In some embodiments, the ring that includes X¹, X², and X³, contains one or two nitrogen atoms and no other heteroatoms. In some embodiments, the ring that includes X¹, X², and X³, contains one nitrogen atom and one oxygen or one sulfur atom, and no other heteroatoms. In some embodiments, the ring that includes X¹, X², and X³, contains one sulfur atom and no other heteroatoms. In some embodiments, the ring that includes X¹, X², and X³, contains one oxygen atom and no other heteroatoms.

In some embodiments, X⁴ is CR⁴. In some embodiments, X⁴ is N. In some embodiments, X⁵ is CR⁵. In some embodiments, X⁵ is N. In some embodiments, X⁶ is CR⁶.

In some embodiments, X⁶ is N. In some embodiments, X⁷ is CH. In some embodiments, X⁷ is CF. In some embodiments, X⁷ is N. In some embodiments, one of X⁴, X⁵, X⁶, and X⁷ are N. In some embodiments, two of X⁴, X⁵, X⁶, and X⁷ are N. In some embodiments, X⁴ is CR⁴; X⁵ is CR⁵; X⁶ is CR⁶; and X⁷ is CH. In some embodiments, R^{1A} is C1-C6 alkyl. In some embodiments, R^{1A} is methyl.

In some embodiments, the ring that includes X⁴, X⁵, X⁶, and X⁷, contains one or two nitrogen atoms. In some embodiments, the ring that includes X⁴, X⁵, X⁶, and X⁷, contains one nitrogen atom. In some embodiments, the ring that includes X⁴, X⁵, X⁶, and X⁷, contains two nitrogen atoms. In some embodiments, the ring that includes X⁴, X⁵, X⁶, and X⁷, contains no nitrogen atoms In some embodiments, R^{1A} is —(C0-C6 alkyl)-5-6 membered heteroaryl. In some embodiments, R^{1A} is —(C1-C3 alkyl)-5-6 membered heteroaryl. In some embodiments, R^{1A} is —(CH₂)-5-6 membered heteroaryl. In some embodiments, R^{1A} is —(C0 alkyl)-5-6 membered heteroaryl, e.g., a -5-6 membered heteroaryl, where “C0 alkyl” represents a bond. In some embodiments, R^{1A} is

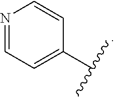 [SMILES: *c1ccncc1]

In some embodiments, R^{1A} is pyridinyl, pyrimidinyl, pyridazinyl, pyrazinyl, oxazolyl, pyrazolyl, thiazolyl, imidazolyl, isoxazolyl, isothiazolyl, or pyrrolyl. In some embodiments, R^{1A} is pyridinyl. In some embodiments, R^{1A} is 2-pyridinyl. In some embodiments, R^{1A} is a 3-pyridinyl. In some embodiments, R^{1A} is a 4-pyridinyl.

In some embodiments, R^{1A} is —C(═O)NR^{F}R^{G}. In some embodiments, R^{1A} is —C(═O)-3-6 membered heterocyclyl optionally substituted with C1-C6 alkyl.

In some embodiments, R^{1A} is —C(═O)-4-6 membered heterocyclyl optionally substituted with C1-C3 alkyl. In some embodiments, R^{1A} is —C(═O)-4-6 membered heterocyclyl substituted with C1-C3 alkyl. In some embodiments, R^{1A} is —C(═O)-4-6 membered heterocyclyl substituted with methyl. In some embodiments, R^{1A} is an unsubstituted —C(═O)-4-6 membered heterocyclyl. In some embodiments, the 4-6 membered heterocyclyl of R^{1A} is azetidinyl, oxetanyl, pyrrolidinyl, tetrahydrofuranyl, piperidinyl, tetrahydropyranyl, piperazinyl, or morpholinyl. In some embodiments, R^{1A} is

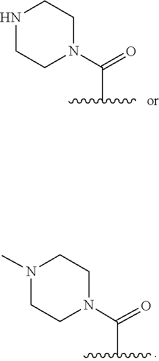

In some embodiments, R^{1A} is phenyl optionally substituted with halogen or —CO₂H. In some embodiments, R^{1A} is phenyl substituted with halogen. In some embodiments, R^{1A} is phenyl substituted with fluoro. In some embodiments, R^{1A} is phenyl substituted with —CO₂H. In some embodiments, R^{1A} is unsubstituted phenyl.

In some embodiments, R^{1A} is 3-6 membered heterocyclyl optionally substituted with C1-C6 alkyl or —S(O₂)—C1-C6 alkyl. In some embodiments, R^{1A} is 3-6 membered heterocyclyl substituted with C1-C6 alkyl or —S(O₂)CH₃. In some embodiments, R^{1A} is 3-6 membered heterocyclyl substituted with C1-C6 alkyl. In some embodiments, R^{1A} is 3-6 membered heterocyclyl substituted with methyl. In some embodiments, R^{1A} is 3-6 membered heterocyclyl optionally substituted with —S(O₂)—C1-C6 alkyl. In some embodiments, R^{1A} is 3-6 membered heterocyclyl substituted with —S(O₂)CH₃.

In some embodiments, R^{1A} is an unsubstituted 3-6 membered heterocyclyl. In some embodiments, R^{1A} is piperidinyl, piperazinyl, dihydropyridinyl, morpholinyl, pyrrolidinyl, azetidinyl, tetrahydropyranyl, tetrahydrofuranyl, or oxetanyl. In some embodiments, R^{1A} is 3-6 unsubstituted membered heterocyclyl. In some embodiments, R^{1A} is piperidinyl. In some embodiments, R^{1A} is 2-piperidinyl. In some embodiments, R^{1A} is 3-piperidinyl. In some embodiments, R^{1A} is 4-piperidinyl.

In some embodiments, R^{1A} is

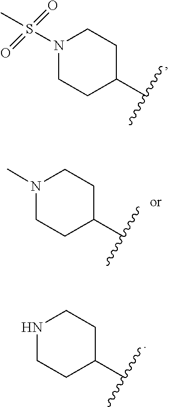

In some embodiments, R^{1A} is piperidinyl, pyrrolidyl, or azetidinyl, substituted with methyl or substituted with —S(O₂)CH₃. In some embodiments, R^{1A} is piperidinyl, pyrrolidinyl, or azetidinyl substituted with methyl. In some embodiments, R^{1A} is piperidinyl, pyrrolidinyl, or azetidinyl, substituted with methyl or substituted with —S(O₂)CH₃.

In some embodiments, R^{1A} is piperidinyl substituted with methyl. In some embodiments, R^{1A} is 2-piperidinyl substituted with methyl. In some embodiments, R^{1A} is 3-piperidinyl substituted with methyl. In some embodiments, R^{1A} is 4-piperidinyl substituted with methyl.

In some embodiments, R^{1A} is piperidinyl substituted with —S(O₂)CH₃. In some embodiments, R^{1A} is piperidinyl substituted with —S(O₂)CH₃. In some embodiments, R^{1A} is 2-piperidinyl substituted with —S(O₂)CH₃. In some embodiments, R^{1A} is 3-piperidinyl substituted with —S(O₂)CH₃. In some embodiments, R^{1A} is 4-piperidinyl substituted with —S(O₂)CH₃.

In some embodiments, R^{1A} is a C3-C6 cycloalkyl optionally substituted with hydroxyl, —C(═O)NR^{F}R^{G}, —NR^{F}R^{G}, —CO₂R^{H}, or C1-C6 alkoxy.

In some embodiments, R^{1A} is a C3-C6 cycloalkyl substituted with hydroxyl, —C(═O)NR^{F}R^{G}, —NR^{F}R^{G}, —CO₂R^{H}, or C1-C6 alkoxy.

In some embodiments, R^{A} is an unsubstituted C3-C6 cycloalkyl.

In some embodiments, R^{1A} is

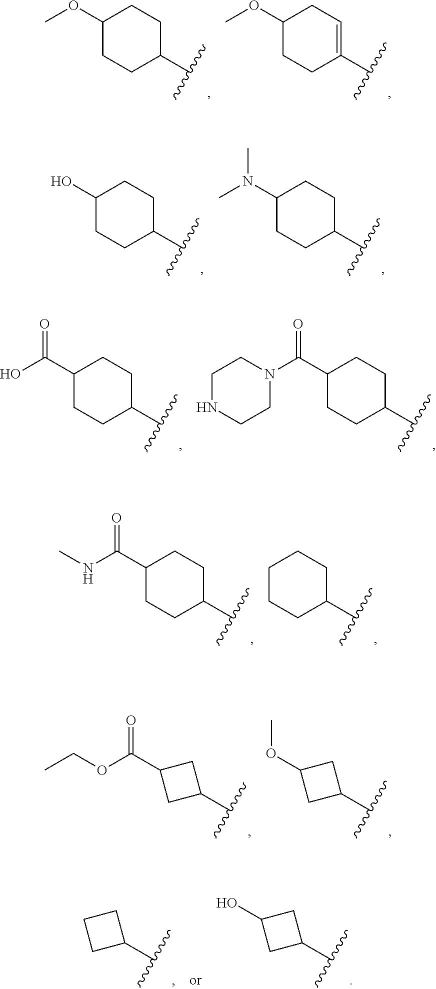

In some embodiments, R^{1A} is hydrogen.

In some embodiments, R^{1B} is hydrogen. In some embodiments, R^{1B} is absent.

In some embodiments, R^{2A} is hydrogen. In some embodiments, R^{2A} is C1-C6 alkyl. In some embodiments, R^{2A} is methyl. In some embodiments, R^{2A} is C1-C6 haloalkyl. In some embodiments, R^{2A} is trifluoromethyl. In some embodiments, R^{2A} is C3-C6 cycloalkyl.

In some embodiments, R^{2A} is 5-6 membered heteroaryl. In some embodiments, R^{2A} is

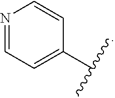 [SMILES: *c1ccncc1]

In some embodiments, R^{2A} is pyridinyl, pyrimidinyl, pyridazinyl, pyrazinyl, oxazolyl, pyrazolyl, thiazolyl, imidazolyl, isoxazolyl, isothiazolyl, or pyrrolyl. In some embodiments, R^{2A} is a pyridinyl. In some embodiments, R^{2A} is 2-pyridinyl. In some embodiments, R^{2A} is a 3-pyridinyl. In some embodiments, R^{2A} is a 4-pyridinyl.

In some embodiments, R^{2A} is phenyl optionally substituted with —CO₂H. In some embodiments, R^{2A} is phenyl substituted with —CO₂H. In some embodiments, R^{2A} is unsubstituted phenyl.

In some embodiments, R^{2A} is a 3-6 membered heterocyclyl optionally substituted with C1-C6 alkyl, C1-C6 haloalkyl, C1-C6 hydroxyalkyl, or —(C0-C6 alkyl)-3-6 membered cycloalkyl optionally substituted with —CO₂H. In some embodiments, R^{2A} is a 3-6 membered heterocyclyl substituted with C1-C6 alkyl, C1-C6 haloalkyl, C1-C6 hydroxyalkyl, or −(C0-C6 alkyl)-3-6 membered cycloalkyl substituted with —CO₂H. In some embodiments, R^{2A} is a 3-6 membered heterocyclyl substituted with C1-C6 alkyl, C1-C6 haloalkyl, C1-C6 hydroxyalkyl, or an unsubstituted —(C0-C6 alkyl)-3-6 membered cycloalkyl.

In some embodiments, R^{2A} is a 3-6 membered heterocyclyl substituted with C1-C6 alkyl. In some embodiments, R^{2A} is a 3-6 membered heterocyclyl substituted with C1-C3 alkyl.

In some embodiments, R^{2A} is a 3-6 membered heterocyclyl substituted with C1-C6 haloalkyl. In some embodiments, R^{2A} is a 3-6 membered heterocyclyl substituted with C1-C3 haloalkyl.

In some embodiments, R^{2A} is a 3-6 membered heterocyclyl substituted with C1-C6 hydroxyalkyl. In some embodiments, R^{2A} is a 3-6 membered heterocyclyl substituted with C1-C3 hydroxyalkyl.

In some embodiments, R^{2A} is a 3-6 membered heterocyclyl substituted with —(C0-C6 alkyl)-3-6 membered cycloalkyl optionally substituted with —CO₂H. In some embodiments, R^{2A} is a 3-6 membered heterocyclyl substituted with —(C0-C6 alkyl)-3-6 membered cycloalkyl substituted with —CO₂H. In some embodiments, R^{2A} is a 3-6 membered heterocyclyl substituted with an unsubstituted —(C0-C6 alkyl)-3-6 membered cycloalkyl. In some embodiments, the 3-6 membered heterocyclyl of R^{2A} is piperidinyl, dihydropyridinyl, pyrrolidinyl, azetidinyl, tetrahydropyranyl, tetrahydrofuranyl, or oxetanyl. In some embodiments, R^{2A} is a 4-6 membered heterocyclyl substituted with —(C0-C6 alkyl)-3-6 membered cycloalkyl optionally substituted with —CO₂H. In some embodiments, R^{2A} is a 4-6 membered heterocyclyl substituted with —(C0-C6 alkyl)-3-6 membered cycloalkyl substituted with —CO₂H. In some embodiments, R^{2A} is a 4-6 membered heterocyclyl substituted with an unsubstituted —(C0-C6 alkyl)-3-6 membered cycloalkyl.

In some embodiments, R^{2A} is an unsubstituted 3-6 membered heterocyclyl. In some embodiments, R^{2A} is piperidinyl, dihydropyridinyl, pyrrolidinyl, azetidinyl, tetrahydropyranyl, tetrahydrofuranyl, or oxetanyl.

In some embodiments, R^{2A} is

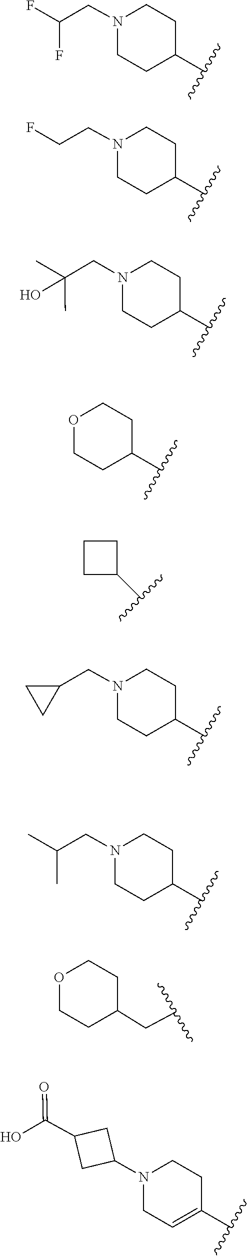

In some embodiments, R²B is hydrogen. In some embodiments, R^{2B} is absent.

In some embodiments, R^{3A} is hydrogen. In some embodiments, R^{3B} is hydrogen. In some embodiments, R^{3B} is C1-C6 alkyl. In some embodiments, R^{3B} is methyl. In some embodiments, R^{3B} is absent.

In some embodiments, R⁴ is hydrogen. In some embodiments, R⁴ is methyl. In some embodiments, R⁴ is 3-6 membered heterocyclyl optionally substituted with C1-C6 alkyl. In some embodiments, R⁴ is 3-6 membered heterocyclyl substituted with C1-C6 alkyl. In some embodiments, R⁴ is 3-6 membered heterocyclyl substituted with methyl. In some embodiments, R⁴ is piperidinyl, dihydropyridinyl, pyrrolidinyl, azetidinyl, tetrahydropyranyl, tetrahydrofuranyl, or oxetanyl; each substituted with C1-C6 alkyl. In some embodiments, R⁴ is an unsubstituted 3-6 membered heterocyclyl. In some embodiments, R⁴ is piperidinyl, dihydropyridinyl, pyrrolidinyl, azetidinyl, tetrahydropyranyl, tetrahydrofuranyl, or oxetanyl.

In some embodiments, R⁴ is piperidinyl optionally substituted with methyl. In some embodiments, R⁴ is piperazinyl optionally substituted with methyl. In some embodiments, R⁴ is 2-piperidinyl. In some embodiments, R⁴ is 3-piperidinyl. In some embodiments, R⁴ is 4-piperidinyl. In some embodiments, R⁴ is 2-piperidinyl substituted with methyl. In some embodiments, R⁴ is 3-piperidinyl substituted with methyl. In some embodiments, R⁴ is 4-piperidinyl substituted with methyl.

In some embodiments, R⁴ is

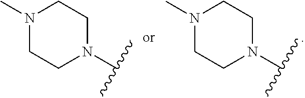

In some embodiments, R⁵ is hydrogen. In some embodiments, R⁵ is —CO₂H. In some embodiments, R⁵ is —C(═O)OCH₃. In some embodiments, R⁵ is C1-C6 alkyl optionally substituted with hydroxyl. In some embodiments, R⁵ is C1-C6 alkyl substituted with hydroxyl.

In some embodiments, R⁵ is —CH₂OH. In some embodiments, R⁵ is an unsubstituted C1-C6 alkyl. In some embodiments, R⁵ is methyl.

In some embodiments, R⁵ is a 5-6 membered heteroaryl optionally substituted with C1-C6 alkyl. In some embodiments, R⁵ is a 5-6 membered heteroaryl substituted with C1-C6 alkyl. In some embodiments, R⁵ is a 5-6 membered heteroaryl substituted with methyl. In some embodiments, R⁵ is pyridinyl, pyrimidinyl, pyridazinyl, pyrazinyl, oxazolyl, pyrazolyl, thiazolyl, imidazolyl, isoxazolyl, isothiazolyl, or pyrrolyl; each substituted with C1-C6 alkyl.

In some embodiments, R⁵ is an unsubstituted 5-6 membered heteroaryl. In some embodiments, R⁵ is pyridinyl, pyrimidinyl, pyridazinyl, pyrazinyl, oxazolyl, pyrazolyl, thiazolyl, imidazolyl, isoxazolyl, isothiazolyl, or pyrrolyl. In some embodiments, R⁵ is oxazolyl. In some embodiments, R⁵ is pyrazolyl optionally substituted with methyl.

In some embodiments, R⁵ is

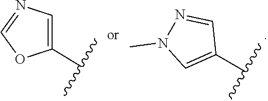

In some embodiments, R⁶ is hydrogen. In some embodiments, R⁶ is a 5-6 membered heteroaryl optionally substituted with 1-2 substituents independently selected from R^{D}, C1-C6 alkyl, and 4-6 membered heterocyclyl optionally substituted with hydroxyl. In some embodiments, R⁶ is a 5-6 membered heteroaryl substituted with 1-2 substituents independently selected from R^{D}, C1-C6 alkyl, and 4-6 membered heterocyclyl optionally substituted with hydroxyl. In some embodiments, R⁶ is a 5-6 membered heteroaryl substituted with 1-2 substituents independently selected from R^{D}, C1-C6 alkyl, and 4-6 membered heterocyclyl substituted with hydroxyl. In some embodiments, R⁶ is an unsubstituted 5-6 membered heteroaryl.

In some embodiments, R⁶ is a 5-6 membered heteroaryl substituted with one substituent selected from R^{D}, C1-C6 alkyl, and a 4-6 membered heterocyclyl optionally substituted with hydroxyl. In some embodiments, R⁶ is a 5-6 membered heteroaryl substituted with 2 substituents independently selected from R^{D}, C1-C6 alkyl, and 4-6 membered heterocyclyl optionally substituted with hydroxyl.

In some embodiments, R⁶ is a 5-6 membered heteroaryl substituted with 1 R^{D}. In some embodiments, R⁶ is a 5-6 membered heteroaryl substituted with 1 or 2 independently selected C1-C6 alkyl. In some embodiments, R⁶ is a 5-6 membered heteroaryl substituted with one C1-C6 alkyl. In some embodiments, R⁶ is a 5-6 membered heteroaryl substituted with 2 independently selected C1-C6 alkyl. In some embodiments, R⁶ is a 5-6 membered heteroaryl substituted with one 4-6 membered heterocyclyl optionally substituted with hydroxyl. In some embodiments, R⁶ is a 5-6 membered heteroaryl substituted with one 4-6 membered heterocyclyl substituted with hydroxyl. In some embodiments, R⁶ is a 5-6 membered heteroaryl substituted with one unsubstituted 4-6 membered heterocyclyl.

In some embodiments, R⁶ is a 5-6 membered heteroaryl substituted with one R^{D} and a 4-6 membered heterocyclyl optionally substituted with hydroxyl. In some embodiments, R⁶ is a 5-6 membered heteroaryl substituted with one C1-C6 alkyl and one R^{D}. In some embodiments, R⁶ is a 5-6 membered heteroaryl substituted with one C1-C6 alkyl and one 4-6 membered heterocyclyl optionally substituted with hydroxyl.

In some embodiments, the 5-6 membered heteroaryl of R⁶ is pyridinyl, pyrimidinyl, pyridazinyl, pyrazinyl, oxazolyl, pyrazolyl, thiazolyl, imidazolyl, isoxazolyl, isothiazolyl, or pyrrolyl. In some embodiments, the 5-6 membered heteroaryl of R⁶ is oxazolyl, thiazolyl, imidazolyl, or pyrazolyl. In some embodiments, the 5-6 membered heteroaryl of R⁶ is oxazolyl. In some embodiments, the 5-6 membered heteroaryl of R⁶ is thiazolyl. In some embodiments, the 5-6 membered heteroaryl of R⁶ is imidazolyl. In some embodiments, the 5-6 membered heteroaryl of R⁶ is pyrazolyl. In some embodiments, the 5-6 membered heteroaryl of R⁶ is pyridinyl, pyrimidinyl, pyridazinyl or pyrazinyl. In some embodiments, the 5-6 membered heteroaryl of R⁶ is pyridinyl. In some embodiments, the 5-6 membered heteroaryl of R⁶ is 2-pyridinyl. In some embodiments, the 5-6 membered heteroaryl of R⁶ is 3-pyridinyl. In some embodiments, the 5-6 membered heteroaryl of R⁶ is 4-pyridinyl.

In some embodiments, R⁶ is

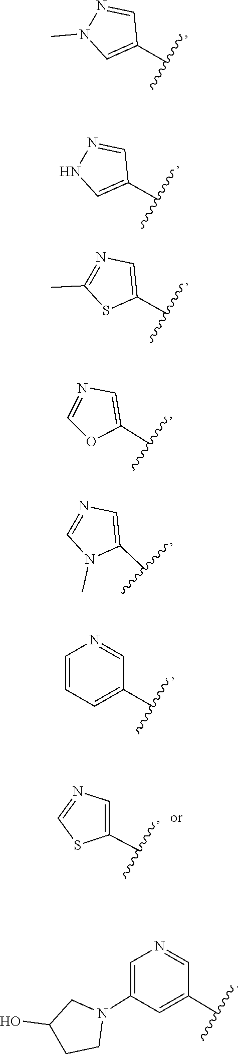

In some embodiments, R^{A} is absent. In some embodiments, R^{A} is hydrogen.

In some embodiments, R^{A} is C1-C6 alkyl optionally substituted with 3-6 membered heterocyclyl or 5-6 membered heteroaryl. In some embodiments, R^{A} is C1-C6 alkyl substituted with 3-6 membered heterocyclyl or 5-6 membered heteroaryl.

In some embodiments, R^{A} is C1-C6 alkyl substituted with 3-6 membered heterocyclyl. In some embodiments, R^{A} is —(CH₂)-4-6 membered heterocyclyl. For example, —(CH₂)-oxetanyl, —(CH₂)-pyrrolidinyl, —(CH₂)-tetrahydrofuranyl, —(CH₂)-tetrahydropyranyl, —(CH₂)-piperidinyl, —(CH₂)-piperazinyl, or —(CH₂)-morpholinyl.

In some embodiments, R^{A} is C1-C6 alkyl substituted with 5-6 membered heteroaryl. In some embodiments, R^{A} is —(CH₂)-5-6 membered heteroaryl. For example, —(CH₂)-pyridinyl, —(CH₂)-pyrimidinyl, —(CH₂)-pyridazinyl, —(CH₂)-pyrazinyl, —(CH₂)-oxazolyl, —(CH₂)-pyrazolyl, —(CH₂)-thiazolyl, —(CH₂)-imidazolyl, —(CH₂)-isoxazolyl, —(CH₂)-isothiazolyl, or —(CH₂)-pyrrolyl. In some embodiments, R^{A} is —(CH₂)-pyridinyl. In some embodiments, R^{A} is —(CH₂)-2-pyridinyl. In some embodiments, R^{A} is —(CH₂)-3-pyridinyl. In some embodiments, R^{A} is —(CH₂)-4-pyridinyl.

In some embodiments, R^{A} is

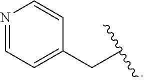 [SMILES: *Cc1ccncc1]

In some embodiments, R^{A} is a C3-C6 cycloalkyl optionally substituted with hydroxyl or C1-C6 alkoxy. In some embodiments, R^{A} is a C3-C6 cycloalkyl substituted with hydroxyl or C1-C6 alkoxy. In some embodiments, R^{A} is a C3-C6 cycloalkyl substituted with hydroxyl.

In some embodiments, R^{A} is a C3-C6 cycloalkyl substituted with C1-C6 alkoxy.

In some embodiments, R^{A} is an unsubstituted C3-C6 cycloalkyl.

In some embodiments, R^{A} is

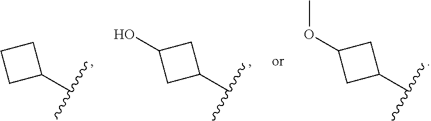

In some embodiments, R^{B} is absent. In some embodiments, R^{B} is hydrogen.

In some embodiments, R^{B} is C1-C6 alkyl optionally substituted with 3-6 membered heterocyclyl or 5-6 membered heteroaryl. In some embodiments, R^{B} is C1-C6 alkyl substituted with 3-6 membered heterocyclyl or 5-6 membered heteroaryl. In some embodiments, R^{B} is an unsubstituted C1-C6 alkyl.

In some embodiments, R^{B} is C1-C6 alkyl optionally substituted with 3-6 membered heterocyclyl or 5-6 membered heteroaryl. In some embodiments, R^{B} is C1-C6 alkyl substituted with 3-6 membered heterocyclyl or 5-6 membered heteroaryl.

In some embodiments, R^{B} is C1-C6 alkyl substituted with 3-6 membered heterocyclyl. In some embodiments, R^{B} is —(CH₂)-4-6 membered heterocyclyl. For example, —(CH₂)-oxetanyl, —(CH₂)-pyrrolidinyl, —(CH₂)-tetrahydrofuranyl, —(CH₂)-tetrahydropyranyl, —(CH₂)-piperidinyl, —(CH₂)-piperazinyl, or —(CH₂)-morpholinyl.

In some embodiments, R^{B} is C1-C6 alkyl substituted with 5-6 membered heteroaryl. In some embodiments, R^{B} is —(CH₂)-5-6 membered heteroaryl. For example, —(CH₂)-pyridinyl, —(CH₂)-pyrimidinyl, —(CH₂)-pyridazinyl, —(CH₂)-pyrazinyl, —(CH₂)-oxazolyl, —(CH₂)-pyrazolyl, —(CH₂)-thiazolyl, —(CH₂)-imidazolyl, —(CH₂)-isoxazolyl, —(CH₂)-isothiazolyl, or —(CH₂)-pyrrolyl. In some embodiments, R^{B} is —(CH₂)-pyridinyl. In some embodiments, R^{B} is —(CH₂)-2-pyridinyl. In some embodiments, R^{B} is —(CH₂)-3-pyridinyl. In some embodiments, R^{B} is —(CH₂)-4-pyridinyl.

In some embodiments, R^{B} is a C3-C6 cycloalkyl optionally substituted with hydroxyl or C1-C6 alkoxy.

In some embodiments, R^{B} is a C3-C6 cycloalkyl substituted with hydroxyl or C1-C6 alkoxy. In some embodiments, R^{B} is an unsubstituted C3-C6 cycloalkyl.

In some embodiments, R^{B} is a C3-C6 cycloalkyl optionally substituted with hydroxyl or C1-C6 alkoxy. In some embodiments, R^{B} is a C3-C6 cycloalkyl substituted with hydroxyl or C1-C6 alkoxy. In some embodiments, R^{B} is a C3-C6 cycloalkyl substituted with hydroxyl.

In some embodiments, R^{B} is a C3-C6 cycloalkyl substituted with C1-C6 alkoxy.

In some embodiments, R^{B} is an unsubstituted C3-C6 cycloalkyl.

In some embodiments, R^{C} is absent. In some embodiments, R^{C} is hydrogen. In some embodiments, R^{C} is methyl.

In some embodiments, R^{D} is

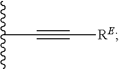

and R^{E} is a C1-C6 alkyl, phenyl, 5-6 membered heteroaryl, or 4-6 membered heterocyclyl; wherein the C1-C6 alkyl, phenyl, 5-6 membered heteroaryl, and 4-6 membered heterocyclyl are each optionally substituted with 1-2 independently selected R^{I}.

In some embodiments, R^{D} is

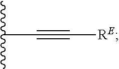

and R^{E} is C1-C16 alky optionally substituted with 1-2 independently selected R^{I}. In some embodiments, R^{D} is

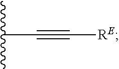

and R^{E} is phenyl optionally substituted with 1-2 independently selected R^{I}. In some embodiments, R^{D} is

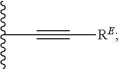

and R^{E} is a 5-6 membered heteroaryl optionally substituted with 1-2 independently selected R^{I}. In some embodiments, R^{D} is

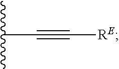

and R^{E} is a 4-6 membered heterocyclyl optionally substituted with 1-2 independently selected R^{I}.

In some embodiments, R^{D}

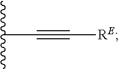

and R^{E} is C1-C6 alkyl substituted with 1-2 independently selected R^{I}. In some embodiments, R^{D} is

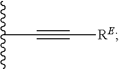

and R^{E} is phenyl substituted with 1-2 independently selected R^{I}. In some embodiments, R^{D} is

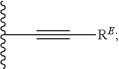

and R^{E} is a 5-6 membered heteroaryl substituted with 1-2 independently selected R^{I}. In some embodiments, R^{D} is

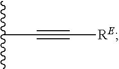

and R^{E} is a 4-6 membered heterocyclyl substituted with 1-2 independently selected R^{I}.

In some embodiments, R^{D}

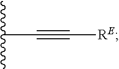

and R^{E} is C1-C6 alkyl substituted with 1 R^{I}. In some embodiments, R^{D} is

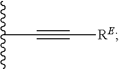

and R^{E} is phenyl substituted with 1 R^{I}. In some embodiments, R^{D} is

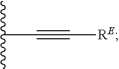

and R^{E} is a 5-6 membered heteroaryl substituted yIn some embodiments, R^{D} is

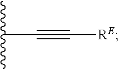

and R^{E} is a 4-6 membered heterocyclyl substituted with 1 R^{I}.

In some embodiments, R^{D} is

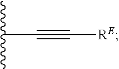

and R^{E} is C1-C6 alkyl substituted with 2 independently selected R^{I}. In some embodiments, R^{D} is

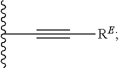

and R^{E} is phenyl substituted with 2 independently selected R^{I}. In some embodiments, R^{D} is

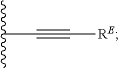

and R^{E} is a 5-6 membered heteroaryl substituted with 2 independently selected R^{I}. In some embodiments, R^{D} is

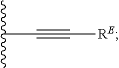

and R^{E} is a 4-6 membered heterocyclyl substituted with 2 independently selected R^{I}.

In some embodiments, R^{D} is

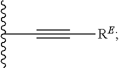

and R^{E} is an unsubstituted C1-C6 alkyl. In some embodiments, R^{D} is

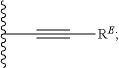

and R^{E} is an unsubstituted phenyl. In some embodiments, R^{D} is

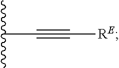

and R^{E} is an unsubstituted phenyl. In some embodiments, R^{D} is

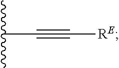

and R^{E} is an unsubstituted 4-6 membered heterocyclyl.

In some embodiments when R^{D} is

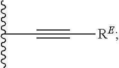

and the R^{E} group is substituted with 1 R^{I}. In some embodiments when R^{D} is

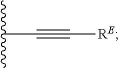

and the R^{E} group is substituted with 2 independently selected R^{I}. In some embodiments when R^{D} is

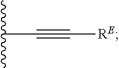

and the R^{E} group is unsubstituted.

In some embodiments, R^{E} is pyridinyl, pyrimidinyl, pyridazinyl, pyrazinyl, oxazolyl, pyrazolyl, thiazolyl, imidazolyl, isoxazolyl, isothiazolyl, or pyrrolyl; each optionally substituted with 1-2 independently selected R^{I}. In some embodiments, R^{E} is pyridinyl, pyrimidinyl, pyridazinyl, pyrazinyl, oxazolyl, pyrazolyl, thiazolyl, imidazolyl, isoxazolyl, isothiazolyl, or pyrrolyl; each optionally substituted with 1-2 independently selected R^{I}. In some embodiments, R^{E} is oxazolyl, thiazolyl, imidazolyl or pyrazolyl; each optionally substituted with 1-2 independently selected R^{I}. In some embodiments, R^{E} is oxazolyl, thiazolyl, imidazolyl or pyrazolyl; each optionally substituted with 1-2 independently selected R^{I}. In some embodiments, R^{E} is pyridinyl, pyrimidinyl, pyridazinyl or pyrazinyl; each optionally substituted with 1-2 independently selected R^{I}. In some embodiments, R^{E} is pyridinyl, pyrimidinyl, pyridazinyl or pyrazinyl; each optionally substituted with 1-2 independently selected R^{I}.

In some embodiments, R^{E} is pyridinyl, pyrimidinyl, pyridazinyl, pyrazinyl, oxazolyl, pyrazolyl, thiazolyl, imidazolyl, isoxazolyl, isothiazolyl, or pyrrolyl; each substituted with 1-2 independently selected R^{I}. In some embodiments, R^{E} is pyridinyl, pyrimidinyl, pyridazinyl, pyrazinyl, oxazolyl, pyrazolyl, thiazolyl, imidazolyl, isoxazolyl, isothiazolyl, or pyrrolyl; each substituted with 1-2 independently selected R^{I}. In some embodiments, R^{E} is oxazolyl, thiazolyl, imidazolyl or pyrazolyl; each substituted with 1-2 independently selected R^{I}. In some embodiments, R^{E} is oxazolyl, thiazolyl, imidazolyl or pyrazolyl; each substituted with 1-2 independently selected R^{I}. In some embodiments, R^{E} is pyridinyl, pyrimidinyl, pyridazinyl or pyrazinyl; each substituted with 1-2 independently selected R^{I}. In some embodiments, R^{E} is pyridinyl, pyrimidinyl, pyridazinyl or pyrazinyl; each optionally substituted with 1-2 independently selected R^{I}.

In some embodiments, R^{E} is pyridinyl, pyrimidinyl, pyridazinyl, pyrazinyl, oxazolyl, pyrazolyl, thiazolyl, imidazolyl, isoxazolyl, isothiazolyl, or pyrrolyl; each substituted with 1 R^{I}. In some embodiments, R^{E} is pyridinyl, pyrimidinyl, pyridazinyl, pyrazinyl, oxazolyl, pyrazolyl, thiazolyl, imidazolyl, isoxazolyl, isothiazolyl, or pyrrolyl; each substituted with 1 R^{I}. In some embodiments, R^{E} is oxazolyl, thiazolyl, imidazolyl or pyrazolyl; each substituted with 1 R^{I}. In some embodiments, R^{E} is oxazolyl, thiazolyl, imidazolyl or pyrazolyl; each substituted with 1 R^{I}. In some embodiments, R^{E} is pyridinyl, pyrimidinyl, pyridazinyl or pyrazinyl; each substituted with 1 R^{I}. In some embodiments, R^{E} is pyridinyl, pyrimidinyl, pyridazinyl or pyrazinyl; each optionally substituted with 1 R^{I}.

In some embodiments, R^{E} is pyridinyl, pyrimidinyl, pyridazinyl, pyrazinyl, oxazolyl, pyrazolyl, thiazolyl, imidazolyl, isoxazolyl, isothiazolyl, or pyrrolyl; each substituted with 2 independently selected R^{I}. In some embodiments, R^{E} is pyridinyl, pyrimidinyl, pyridazinyl, pyrazinyl, oxazolyl, pyrazolyl, thiazolyl, imidazolyl, isoxazolyl, isothiazolyl, or pyrrolyl; each substituted with 2 independently selected R^{I}. In some embodiments, R^{E} is oxazolyl, thiazolyl, imidazolyl or pyrazolyl; each substituted with 2 independently selected R^{I}. In some embodiments, R^{E} is oxazolyl, thiazolyl, imidazolyl or pyrazolyl; each substituted with 2 independently selected R^{I}. In some embodiments, R^{E} is pyridinyl, pyrimidinyl, pyridazinyl or pyrazinyl; each substituted with 2 independently selected R^{I}. In some embodiments, R^{E} is pyridinyl, pyrimidinyl, pyridazinyl or pyrazinyl; each optionally substituted with 2 independently selected R^{I}.

In some embodiments, R^{E} is an unsubstituted oxazolyl, thiazolyl, imidazolyl or pyrazolyl. In some embodiments, R^{E} is an unsubstituted oxazolyl, thiazolyl, imidazolyl or pyrazolyl. In some embodiments, R^{E} is an unsubstituted pyridinyl, pyrimidinyl, pyridazinyl or pyrazinyl. In some embodiments, R^{E} is an unsubstituted pyridinyl, pyrimidinyl, pyridazinyl or pyrazinyl.

In some embodiments, R^{E} is piperidinyl, piperazinyl, morpholinyl, pyrrolidinyl, azetidinyl, tetrahydropyranyl, tetrahydrofuranyl, or oxetanyl; each optionally substituted with 1-2 independently selected R^{I}. In some embodiments, R^{E} is piperidinyl optionally substituted with 1-2 independently selected R^{I}. In some embodiments, R^{E} is 2-piperidinyl optionally substituted with 1-2 independently selected R^{I}. In some embodiments, R^{E} is 3-piperidinyl optionally substituted with 1-2 independently selected R^{I}. In some embodiments, R^{E} is 4-piperidinyl optionally substituted with 1-2 independently selected R^{I}. In some embodiments, R^{E} is tetrahydroypyanyl optionally substituted with 1-2 independently selected R^{I}. In some embodiments, R^{E} is 2-tetrahydroypyanyl optionally substituted with 1-2 independently selected R^{I}. In some embodiments, R^{E} is 3-tetrahydroypyanyl optionally substituted with 1-2 independently selected R^{I}. In some embodiments, R^{E} is 4-tetrahydroypyanyl optionally substituted with 1-2 independently selected R^{I}.

In some embodiments, R^{E} is piperidinyl, piperazinyl, morpholinyl, pyrrolidinyl, azetidinyl, tetrahydropyranyl, tetrahydrofuranyl, or oxetanyl; each substituted with 1-2 independently selected R^{I}. In some embodiments, R^{E} is piperidinyl substituted with 1-2 independently selected R^{I}. In some embodiments, R^{E} is 2-piperidinyl substituted with 1-2 independently selected R^{I}. In some embodiments, R^{E} is 3-piperidinyl substituted with 1-2 independently selected R^{I}. In some embodiments, R^{E} is 4-piperidinyl substituted with 1-2 independently selected R^{I}. In some embodiments, R^{E} is tetrahydroypyanyl substituted with 1-2 independently selected R^{I}. In some embodiments, R^{E} is 2-tetrahydroypyanyl substituted with 1-2 independently selected R^{I}. In some embodiments, R^{E} is 3-tetrahydroypyanyl substituted with 1-2 independently selected R^{I}. In some embodiments, R^{E} is 4-tetrahydroypyanyl substituted with 1-2 independently selected R^{I}.

In some embodiments, R^{E} is piperidinyl, piperazinyl, morpholinyl, pyrrolidinyl, azetidinyl, tetrahydropyranyl, tetrahydrofuranyl, or oxetanyl; each substituted with 1 R^{I}. In some embodiments, R^{E} is piperidinyl substituted with 1 R^{I}. In some embodiments, R^{E} is 2-piperidinyl substituted with 1 R^{I}. In some embodiments, R^{E} is 3-piperidinyl substituted with 1 R^{I}. In some embodiments, R^{E} is 4-piperidinyl substituted with 1 R^{I}. In some embodiments, R^{E} is tetrahydroypyanyl substituted with 1 R^{I}. In some embodiments, R^{E} is 2-tetrahydroypyanyl substituted with 1 R^{I}. In some embodiments, R^{E} is 3-tetrahydroypyanyl substituted with 1 R^{I}. In some embodiments, R^{E} is 4-tetrahydroypyanyl substituted with 1 R^{I}.

In some embodiments, R^{E} is piperidinyl, piperazinyl, morpholinyl, pyrrolidinyl, azetidinyl, tetrahydropyranyl, tetrahydrofuranyl, or oxetanyl; each substituted with 2 independently selected R^{I}. In some embodiments, R^{E} is piperidinyl substituted with 2 independently selected R^{I}. In some embodiments, R^{E} is 2-piperidinyl substituted with 2 independently selected R^{I}. In some embodiments, R^{E} is 3-piperidinyl substituted with 2 independently selected R^{I}. In some embodiments, R^{E} is 4-piperidinyl substituted with 2 independently selected R^{I}. In some embodiments, R^{E} is tetrahydroypyanyl substituted with 2 independently selected R^{I}. In some embodiments, R^{E} is 2-tetrahydroypyanyl substituted with 2 independently selected R^{I}. In some embodiments, R^{E} is 3-tetrahydroypyanyl substituted with 1-2 independently selected R^{I}. In some embodiments, R^{E} is 4-tetrahydroypyanyl substituted with 1-2 independently selected R^{I}.

In some embodiments, R^{E} is an unsubstituted piperidinyl, piperazinyl, morpholinyl, pyrrolidinyl, azetidinyl, tetrahydropyranyl, tetrahydrofuranyl, or oxetanyl. In some embodiments, R^{E} is an unsubstituted piperidinyl. In some embodiments, R^{E} is an unsubstituted 2-piperidinyl. In some embodiments, R^{E} is an unsubstituted 3-piperidinyl. In some embodiments, R^{E} is an unsubstituted 4-piperidinyl. In some embodiments, R^{E} is an unsubstituted tetrahydroypyanyl. In some embodiments, R^{E} is an unsubstituted 2-tetrahydroypyanyl. In some embodiments, R^{E} is an unsubstituted 3-tetrahydroypyanyl. In some embodiments, R^{E} is an unsubstituted 4-tetrahydroypyanyl.

In some embodiments, R^{D} is

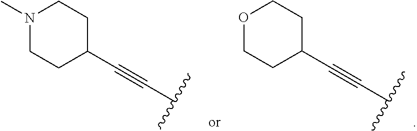

In some embodiments, each R^{F} and R^{G} are independently selected from hydrogen and C1-C6 alkyl. In some embodiments, one of R^{F} and R^{G} is hydrogen and the other of R^{F} and R^{G} is C1-C6 alkyl. In some embodiments, one of R^{F} and R^{G} is hydrogen and the other of R^{F} and R^{G} is methyl. In some embodiments, both R^{F} and R^{G} are hydrogen. In some embodiments, both R^{F} and R^{G} are C1-C6 alkyl. In some embodiments, both R^{F} and R^{G} are methyl.

In some embodiments, R^{F} and R^{G} together with the nitrogen atom to which they are attached form a 4-6 membered heterocyclyl optionally substituted with C1-C6 alkyl. In some embodiments, R^{F} and R^{G} together with the nitrogen atom to which they are attached form a 4-6 membered heterocyclyl substituted with C1-C6 alkyl. In some embodiments, R^{F} and R^{G} together with the nitrogen atom to which they are attached form a 4-6 membered heterocyclyl substituted with methyl. In some embodiments, R^{F} and R^{G} together with the nitrogen atom to which they are attached form an unsubstituted 4-6 membered heterocyclyl.

In some embodiments, R^{F} and R^{G} together with the nitrogen atom to which they are attached form

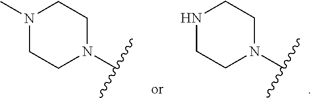

In some embodiments, R^{H} is hydrogen. In some embodiments, R^{H} is C1-C6 alkyl. In some embodiments, R^{H} is C1-C3 alkyl. In some embodiments, R^{H} is methyl.

In some embodiments, R^{I} is C1-C6 alkyl, C1-C6 alkoxy, halogen, hydroxyl, cyano, or trifluoromethyl. In some embodiments, R^{I} is C1-C3 alkyl, C1-C3 alkoxy, halogen, hydroxyl, cyano, or trifluoromethyl. In some embodiments, R^{I} is methyl, methoxy, fluoro, chloro, hydroxyl, cyano, or trifluoromethyl.

In some embodiments, R^{I} is C1-C6 alkyl. In some embodiments, R^{I} is C1-C3 alkyl. In some embodiments, R^{I} is methyl. In some embodiments, R^{I} is C1-C6 alkoxy. In some embodiments, R^{I} is C1-C3 alkoxy. In some embodiments, R^{I} is methoxy. In some embodiments, R^{I} is halogen. In some embodiments, R^{I} is fluoro or chloro. In some embodiments, R^{I} is fluoro. In some embodiments, R^{I} is chloro. In some embodiments, R^{I} is hydroxyl. In some embodiments, R^{I} is cyano. In some embodiments, R^{I} is trifluoromethyl.

In some embodiments, the compound of Formula (I) has the structure of Formula (I-A):

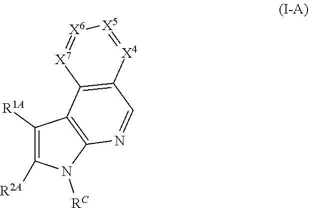

or a pharmaceutically acceptable salt thereof. The definitions of R^{1A}, R^{2A}, R^{C}, X⁴, X⁵, X⁶, and X⁷ are as described in Formula (I). In some embodiments, one of X⁴, X⁵, X⁶, and X⁷ is a nitrogen atom. In some embodiments, two of X⁴, X⁵, X⁶, and X⁷ are nitrogen atoms. In some embodiments, each of X⁴, X⁵, X⁶, and X⁷ is carbon (e.g., CR⁴, and the like).

In some embodiments, the compound of Formula (I) has the structure of Formula (I-B):

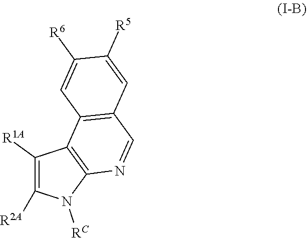

or a pharmaceutically acceptable salt thereof. The definitions of R^{1A}, R^{2A}, R^{C}, R⁵, and R⁶ are as described in Formula (I). In some embodiments, one of R⁵ and R⁶ is hydrogen, and the other of R⁵ and R⁶ is not hydrogen.

In some embodiments, the compound of Formula (I) has the structure of Formula (I-C):

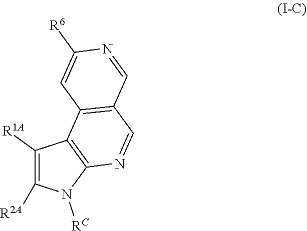

or a pharmaceutically acceptable salt thereof. The definitions of R^{1A}, R^{2A}, R^{C}, and R⁶ are as described in Formula (I).

In some embodiments, the compound of Formula (I) has the structure of Formula (I-D):

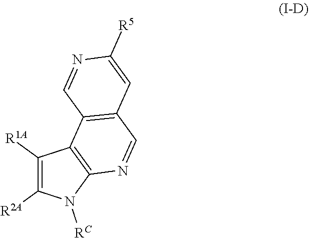

or a pharmaceutically acceptable salt thereof. The definitions of R^{1A}, R^{2A}, R^{C}, and R⁵ are as described in Formula (I).

In some embodiments, the compound of Formula (I) has the structure of Formula (I-E):

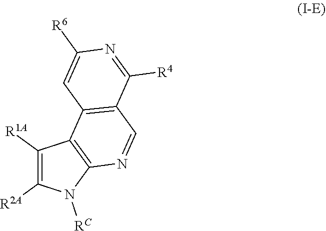

or a pharmaceutically acceptable salt thereof. The definitions of R^{A}, R^{2A}, R^{C}, R⁴, and R⁶ are as described in Formula (I).

In some embodiments, the compound of Formula (I) has the structure of Formula (I-F):

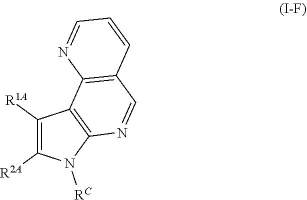

or a pharmaceutically acceptable salt thereof. The definitions of R^{1A}, R^{2A}, and R^{C} are as described in Formula (I).

In some embodiments, the compound of Formula (I) has the structure of Formula (I-G):

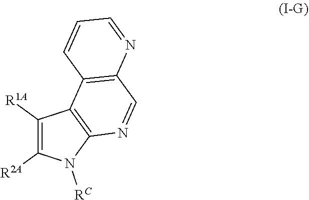

In some embodiments, the compound of Formula (I) has the structure of Formula (I-H):

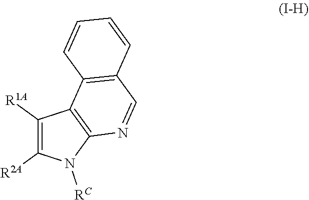

or a pharmaceutically acceptable salt thereof. The definitions of R^{1A}, R^{2A}, and R^{C} are as described in Formula (I).

In some embodiments, the compound of Formula (I) has the structure of Formula (I-I):

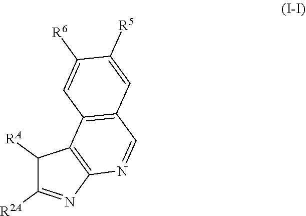

or a pharmaceutically acceptable salt thereof. The definitions of R^{A}, R^{2A}, R⁵, and R⁶ are as described in Formula (I). In some embodiments, one of R⁵ and R⁶ is hydrogen, and the other of R⁵ and R⁶ is not hydrogen.

In some embodiments, the compound of Formula (I) has the structure of Formula (I-J):

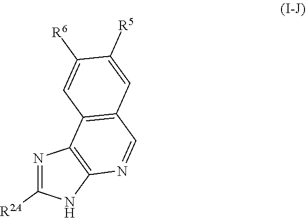

or a pharmaceutically acceptable salt thereof. The definitions of R^{2A}, R⁵, and R⁶ are as described in Formula (I). In some embodiments, one of R⁵ and R⁶ is hydrogen, and the other of R⁵ and R⁶ is not hydrogen.

In some embodiments, the compound of Formula (I) has the structure of Formula (I-K):

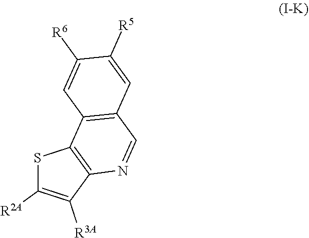

or a pharmaceutically acceptable salt thereof. The definitions of R^{2A}, R^{3A}, R^{I}, and R⁶ are as described in Formula (I). In some embodiments, one of R⁵ and R⁶ is hydrogen, and the other of R⁵ and R⁶ is not hydrogen.

In some embodiments, the compound of Formula (I) has the structure of Formula (I-L):

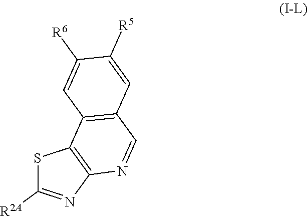

or a pharmaceutically acceptable salt thereof. The definitions of R^{2A}, R⁵, and R⁶ are as described in Formula (I). In some embodiments, one of R⁵ and R⁶ is hydrogen, and the other of R⁵ and R⁶ is not hydrogen.

In some embodiments, the compound of Formula (I) has the structure of Formula (I-M):

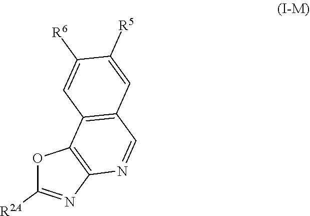

or a pharmaceutically acceptable salt thereof. The definitions of R^{2A}, R⁵, and R⁶ are as described in Formula (I). In some embodiments, one of R⁵ and R⁶ is hydrogen, and the other of R⁵ and R⁶ is not hydrogen.

In some embodiments, the compound of Formula (I) has the structure of Formula (I-N):

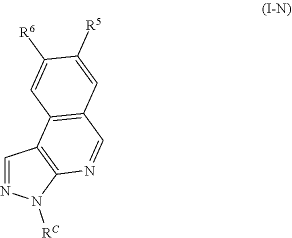

or a pharmaceutically acceptable salt thereof. The definitions of R^{C}, R⁵, and R⁶ are as described in Formula (I). In some embodiments, one of R⁵ and R⁶ is hydrogen, and the other of R⁵ and R⁶ is not hydrogen.

In some embodiments, the compound of Formula (I) has the structure of Formula (I-O):

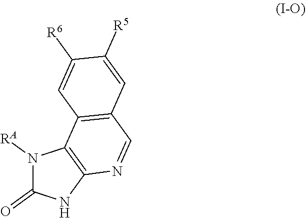

or a pharmaceutically acceptable salt thereof. The definitions of R^{A}, R⁵, and R⁶ are as described in Formula (I). In some embodiments, one of R⁵ and R⁶ is hydrogen, and the other of R⁵ and R⁶ is not hydrogen.

In some embodiments, the compound of Formula (I) has the structure of Formula (I-P):

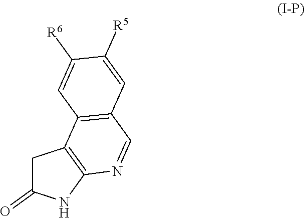

or a pharmaceutically acceptable salt thereof. The definitions of R⁵ and R⁶ are as described in Formula (I). In some embodiments, one of R⁵ and R⁶ is hydrogen, and the other of R⁵ and R⁶ is not hydrogen.

In some embodiments, the compound of Formula (I) has the structure of Formula (I-Q):

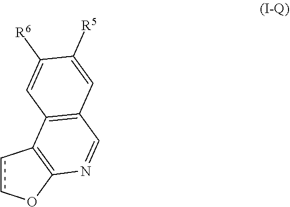

or a pharmaceutically acceptable salt thereof. The definitions of R⁵ and R⁶ are as described in Formula (I). In some embodiments, one of R⁵ and R⁶ is hydrogen, and the other of R⁵ and R⁶ is not hydrogen. In some embodiments, the dashed line in Formula (I-Q) is a single bond. In some embodiments, the dashed line in Formula (I-Q) is a double bond.

In some embodiments, the compound of Formula (I) is selected from a compound in Table 1, or a pharmaceutically acceptable salt thereof.

TABLE 1
Exemplary Compounds of Formula (I)

| Compound | |
|---|---|
| No. | Structure |
| 1 | 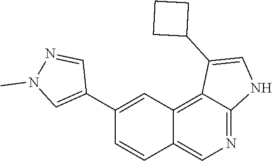 [SMILES: Cn1cc(-c2ccc3cnc4[nH]cc(C5CCC5)c4c3c2)cn1] |
| 2 | 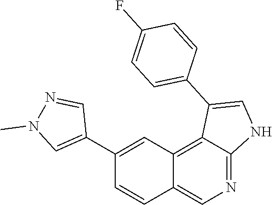 [SMILES: Cn1cc(-c2ccc3cnc4[nH]cc(-c5ccc(F)cc5)c4c3c2)cn1] |
| 3 | 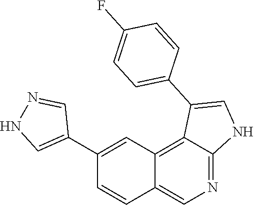 [SMILES: Fc1ccc(-c2c[nH]c3ncc4ccc(-c5cn[nH]c5)cc4c23)cc1] |
| 4 | 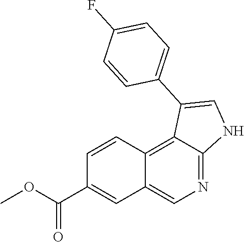 [SMILES: COC(=O)c1ccc2c(cnc3[nH]cc(-c4ccc(F)cc4)c32)c1] |
| 5 | 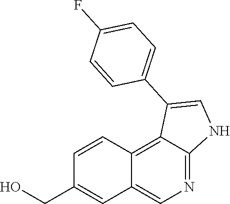 [SMILES: OCc1ccc2c(cnc3[nH]cc(-c4ccc(F)cc4)c32)c1] |
| 6 | 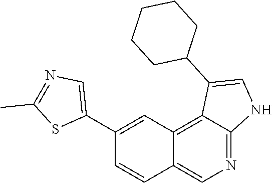 [SMILES: Cc1ncc(-c2ccc3cnc4[nH]cc(C5CCCCC5)c4c3c2)s1] |
| 7 | 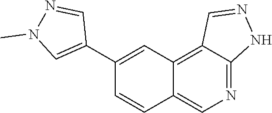 [SMILES: Cn1cc(-c2ccc3cnc4[nH]ncc4c3c2)cn1] |
| 8 | 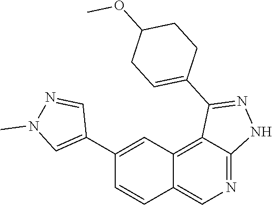 [SMILES: COC1CC=C(c2n[nH]c3ncc4ccc(-c5cnn(C)c5)cc4c23)CC1] |
| 9 | 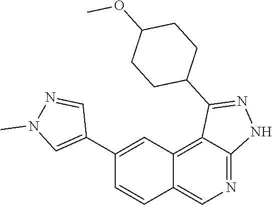 [SMILES: COC1CCC(c2n[nH]c3ncc4ccc(-c5cnn(C)c5)cc4c23)CC1] |
| 10 | 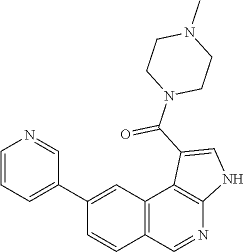 [SMILES: CN1CCN(C(=O)c2c[nH]c3ncc4ccc(-c5cccnc5)cc4c23)CC1] |
| 11 | 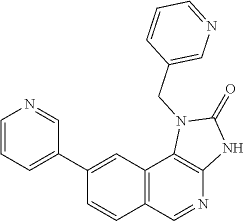 [SMILES: O=c1[nH]c2ncc3ccc(-c4cccnc4)cc3c2n1Cc1cccnc1] |
| 12 | 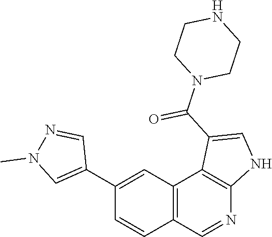 [SMILES: Cn1cc(-c2ccc3cnc4[nH]cc(C(=O)N5CCNCC5)c4c3c2)cn1] |
| 13 | 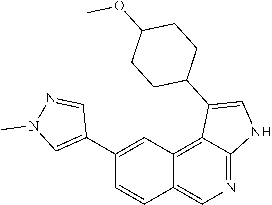 [SMILES: COC1CCC(c2c[nH]c3ncc4ccc(-c5cnn(C)c5)cc4c23)CC1] |
| 14 | 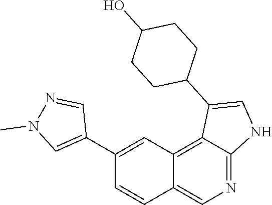 [SMILES: Cn1cc(-c2ccc3cnc4[nH]cc(C5CCC(O)CC5)c4c3c2)cn1] |
| 15 | 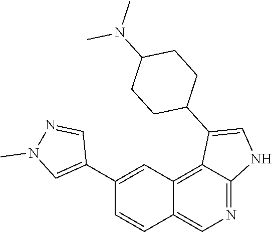 [SMILES: CN(C)C1CCC(c2c[nH]c3ncc4ccc(-c5cnn(C)c5)cc4c23)CC1] |
| 16 | 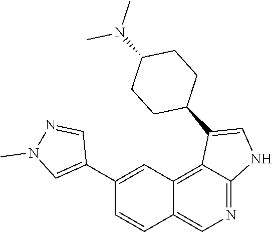 [SMILES: Cn1cc(-c2ccc3cnc4[nH]cc([C@H]5CC[C@H](N(C)C)CC5)c4c3c2)cn1] |
| 17 | 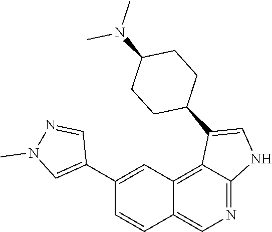 [SMILES: Cn1cc(-c2ccc3cnc4[nH]cc([C@H]5CC[C@@H](N(C)C)CC5)c4c3c2)cn1] |
| 18 | 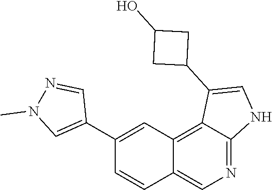 [SMILES: Cn1cc(-c2ccc3cnc4[nH]cc(C5CC(O)C5)c4c3c2)cn1] |
| 19 | 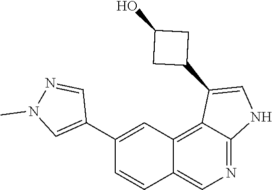 |
| 20 | 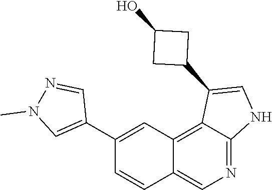 |
| 21 | 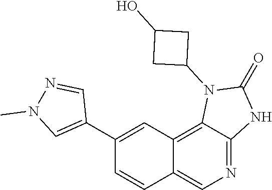 [SMILES: Cn1cc(-c2ccc3cnc4[nH]c(=O)n(C5CC(O)C5)c4c3c2)cn1] |
| 22 | 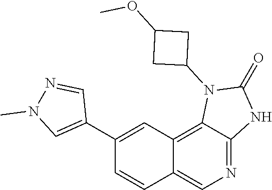 [SMILES: COC1CC(n2c(=O)[nH]c3ncc4ccc(-c5cnn(C)c5)cc4c32)C1] |
| 23 | 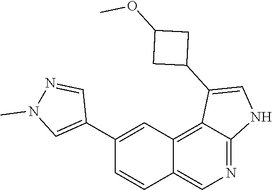 [SMILES: COC1CC(c2c[nH]c3ncc4ccc(-c5cnn(C)c5)cc4c23)C1] |
| 24 | 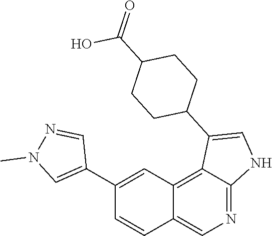 [SMILES: Cn1cc(-c2ccc3cnc4[nH]cc(C5CCC(C(=O)O)CC5)c4c3c2)cn1] |
| 25 | 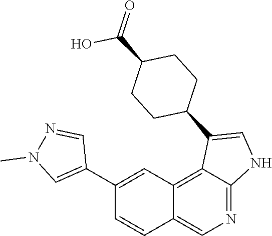 [SMILES: Cn1cc(-c2ccc3cnc4[nH]cc([C@H]5CC[C@@H](C(=O)O)CC5)c4c3c2)cn1] |
| 26 | 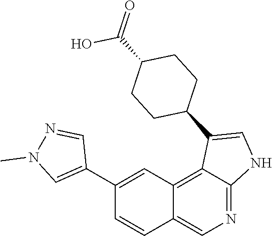 [SMILES: Cn1cc(-c2ccc3cnc4[nH]cc([C@H]5CC[C@H](C(=O)O)CC5)c4c3c2)cn1] |
| 27 | 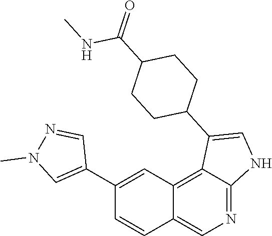 [SMILES: CNC(=O)C1CCC(c2c[nH]c3ncc4ccc(-c5cnn(C)c5)cc4c23)CC1] |
| 28 | 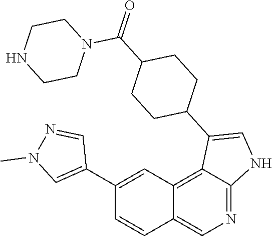 [SMILES: Cn1cc(-c2ccc3cnc4[nH]cc(C5CCC(C(=O)N6CCNCC6)CC5)c4c3c2)cn1] |
| 29 | 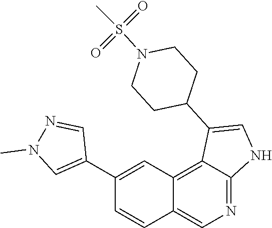 [SMILES: Cn1cc(-c2ccc3cnc4[nH]cc(C5CCN(S(C)(=O)=O)CC5)c4c3c2)cn1] |
| 30 | 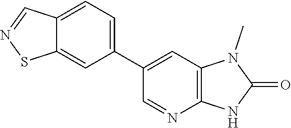 [SMILES: Cn1c(=O)[nH]c2ncc(-c3ccc4cnsc4c3)cc21] |
| 31 | 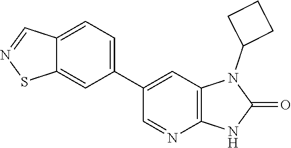 [SMILES: O=c1[nH]c2ncc(-c3ccc4cnsc4c3)cc2n1C1CCC1] |
| 32 | 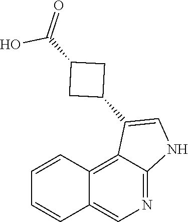 [SMILES: O=C(O)[C@H]1C[C@@H](c2c[nH]c3ncc4ccccc4c32)C1] |
| 33 | 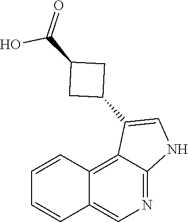 [SMILES: O=C(O)[C@H]1C[C@H](c2c[nH]c3ncc4ccccc4c32)C1] |
| 34 | 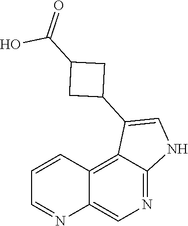 [SMILES: O=C(O)C1CC(c2c[nH]c3ncc4ncccc4c23)C1] |
| 35 | 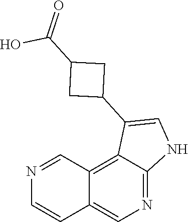 [SMILES: O=C(O)C1CC(c2c[nH]c3ncc4ccncc4c23)C1] |
| 36 | 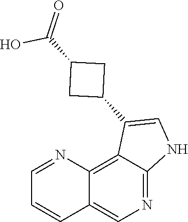 [SMILES: O=C(O)[C@H]1C[C@@H](c2c[nH]c3ncc4cccnc4c32)C1] |
| 37 | 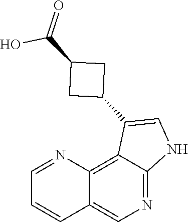 [SMILES: O=C(O)[C@H]1C[C@H](c2c[nH]c3ncc4cccnc4c32)C1] |
| 38 | 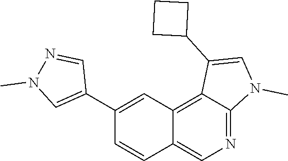 |
| 39 | 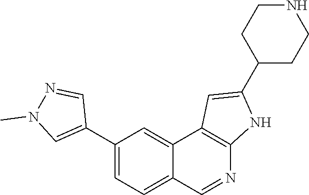 [SMILES: Cn1cc(-c2ccc3cnc4[nH]c(C5CCNCC5)cc4c3c2)cn1] |
| 40 | 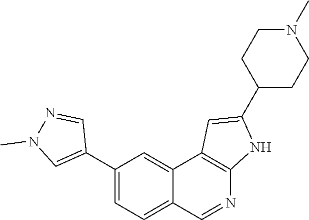 |
| 41 | 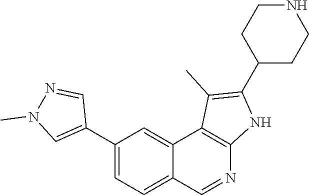 [SMILES: Cc1c(C2CCNCC2)[nH]c2ncc3ccc(-c4cnn(C)c4)cc3c12] |
| 42 | 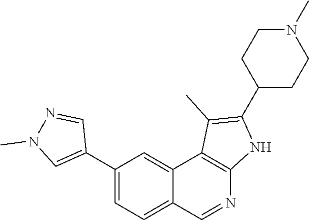 |
| 43 | 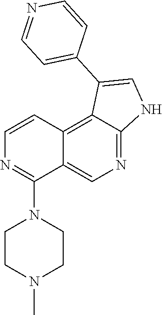 [SMILES: CN1CCN(c2nccc3c2cnc2[nH]cc(-c4ccncc4)c23)CC1] |
| 44 | 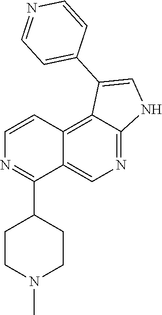 [SMILES: CN1CCC(c2nccc3c2cnc2[nH]cc(-c4ccncc4)c23)CC1] |
| 45 | 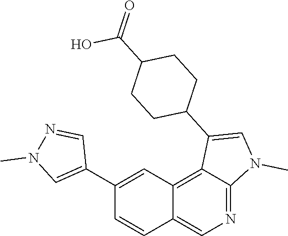 |
| 46 | 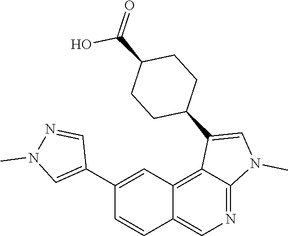 |
| 47 | 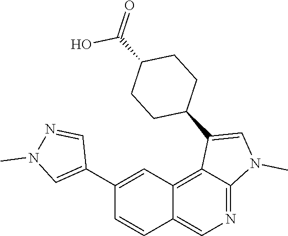 |
| 48 | 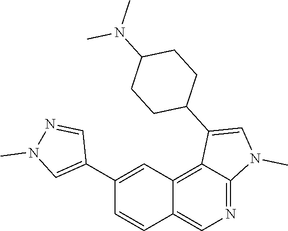 |
| 49 | 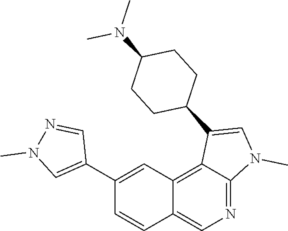 |
| 50 | 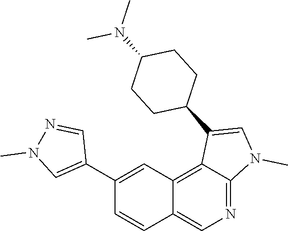 |
| 51 | 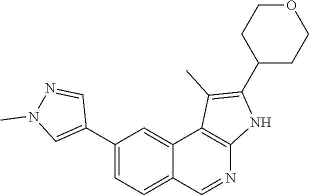 [SMILES: Cc1c(C2CCOCC2)[nH]c2ncc3ccc(-c4cnn(C)c4)cc3c12] |
| 52 | 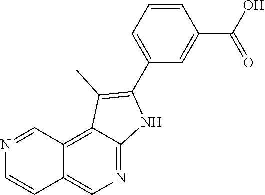 [SMILES: Cc1c(-c2cccc(C(=O)O)c2)[nH]c2ncc3ccncc3c12] |
| 53 | 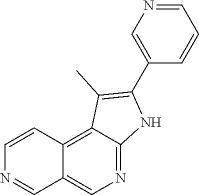 [SMILES: Cc1c(-c2cccnc2)[nH]c2ncc3cnccc3c12] |
| 54 | 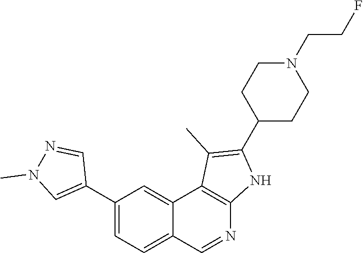 [SMILES: Cc1c(C2CCN(CCF)CC2)[nH]c2ncc3ccc(-c4cnn(C)c4)cc3c12] |
| 55 | 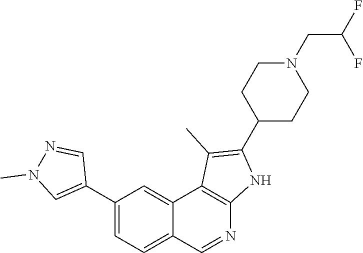 [SMILES: Cc1c(C2CCN(CC(F)F)CC2)[nH]c2ncc3ccc(-c4cnn(C)c4)cc3c12] |
| 56 | 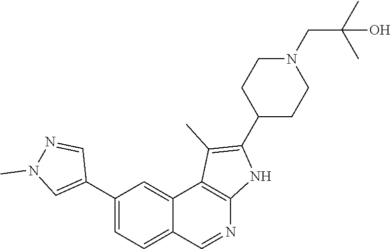 [SMILES: Cc1c(C2CCN(CC(C)(C)O)CC2)[nH]c2ncc3ccc(-c4cnn(C)c4)cc3c12] |
| 57 | 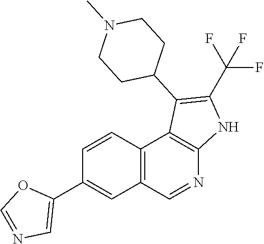 [SMILES: CN1CCC(c2c(C(F)(F)F)[nH]c3ncc4cc(-c5cnco5)ccc4c23)CC1] |
| 58 | 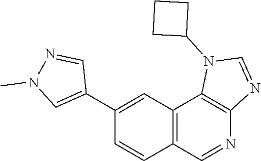 [SMILES: Cn1cc(-c2ccc3cnc4ncn(C5CCC5)c4c3c2)cn1] |
| 59 | 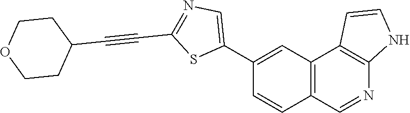 [SMILES: C(#CC1CCOCC1)c1ncc(-c2ccc3cnc4[nH]ccc4c3c2)s1] |
| 60 | 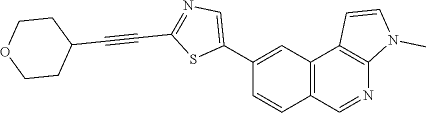 [SMILES: Cn1ccc2c3cc(-c4cnc(C#CC5CCOCC5)s4)ccc3cnc21] |
| 61 | 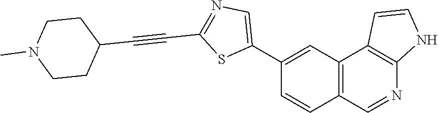 [SMILES: CN1CCC(C#Cc2ncc(-c3ccc4cnc5[nH]ccc5c4c3)s2)CC1] |
| 62 | 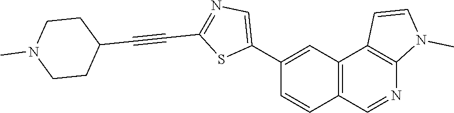 |
| 63 | 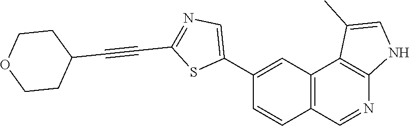 [SMILES: Cc1c[nH]c2ncc3ccc(-c4cnc(C#CC5CCOCC5)s4)cc3c12] |
| 64 | 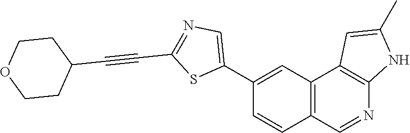 [SMILES: Cc1cc2c(ncc3ccc(-c4cnc(C#CC5CCOCC5)s4)cc32)[nH]1] |
| 65 | 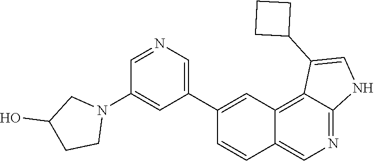 [SMILES: OC1CCN(c2cncc(-c3ccc4cnc5[nH]cc(C6CCC6)c5c4c3)c2)C1] |
| 66 | 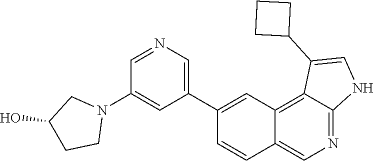 [SMILES: O[C@H]1CCN(c2cncc(-c3ccc4cnc5[nH]cc(C6CCC6)c5c4c3)c2)C1] |
| 67 | 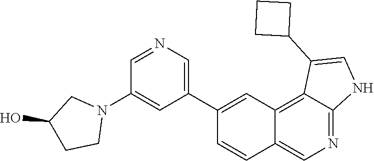 [SMILES: O[C@@H]1CCN(c2cncc(-c3ccc4cnc5[nH]cc(C6CCC6)c5c4c3)c2)C1] |
| 68 | 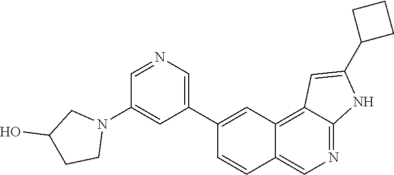 [SMILES: OC1CCN(c2cncc(-c3ccc4cnc5[nH]c(C6CCC6)cc5c4c3)c2)C1] |
| 69 | 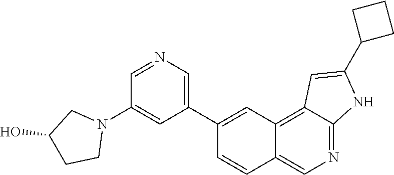 [SMILES: O[C@H]1CCN(c2cncc(-c3ccc4cnc5[nH]c(C6CCC6)cc5c4c3)c2)C1] |
| 70 | 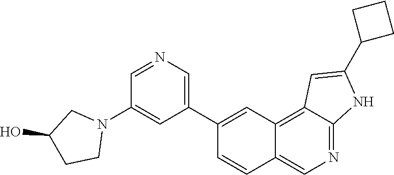 [SMILES: O[C@@H]1CCN(c2cncc(-c3ccc4cnc5[nH]c(C6CCC6)cc5c4c3)c2)C1] |
| 71 | 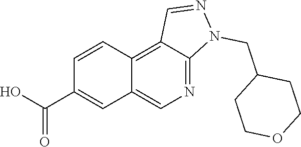 [SMILES: O=C(O)c1ccc2c(cnc3c2cnn3CC2CCOCC2)c1] |
| 72 | 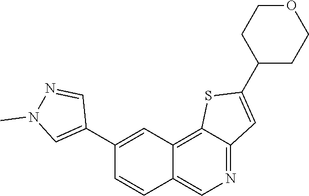 [SMILES: Cn1cc(-c2ccc3cnc4cc(C5CCOCC5)sc4c3c2)cn1] |
| 73 | 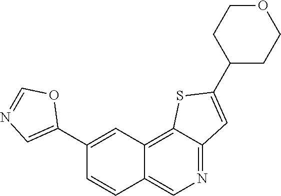 [SMILES: c1ncc(-c2ccc3cnc4cc(C5CCOCC5)sc4c3c2)o1] |
| 74 | 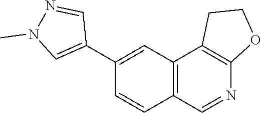 [SMILES: Cn1cc(-c2ccc3cnc4c(c3c2)CCO4)cn1] |
| 75 | 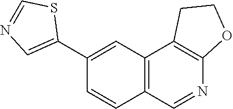 [SMILES: c1ncc(-c2ccc3cnc4c(c3c2)CCO4)s1] |
| 76 | 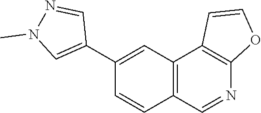 [SMILES: Cn1cc(-c2ccc3cnc4occc4c3c2)cn1] |
| 77 | 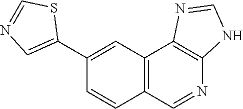 [SMILES: c1nc2c(ncc3ccc(-c4cncs4)cc32)[nH]1] |
| 78 | 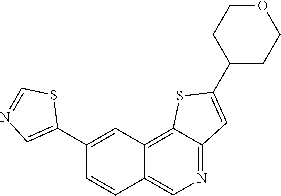 [SMILES: c1ncc(-c2ccc3cnc4cc(C5CCOCC5)sc4c3c2)s1] |
| 79 | 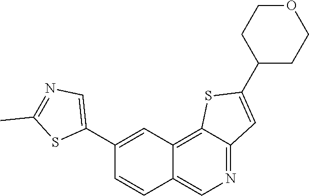 [SMILES: Cc1ncc(-c2ccc3cnc4cc(C5CCOCC5)sc4c3c2)s1] |
| 80 | 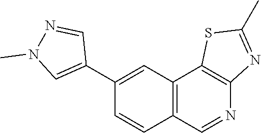 |
| 81 | 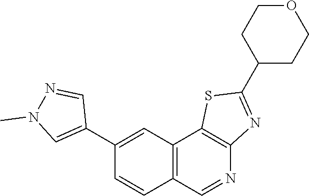 [SMILES: Cn1cc(-c2ccc3cnc4nc(C5CCOCC5)sc4c3c2)cn1] |
| 82 | 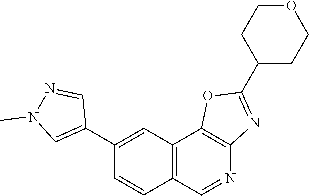 [SMILES: Cn1cc(-c2ccc3cnc4nc(C5CCOCC5)oc4c3c2)cn1] |
| 83 | 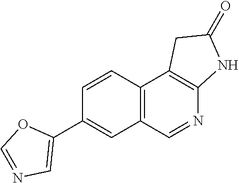 [SMILES: O=C1Cc2c(ncc3cc(-c4cnco4)ccc23)N1] |
| 84 | 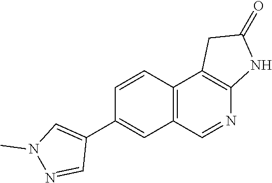 [SMILES: Cn1cc(-c2ccc3c4c(ncc3c2)NC(=O)C4)cn1] |
| 85 | 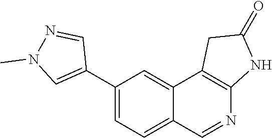 [SMILES: Cn1cc(-c2ccc3cnc4c(c3c2)CC(=O)N4)cn1] |
| 86 | 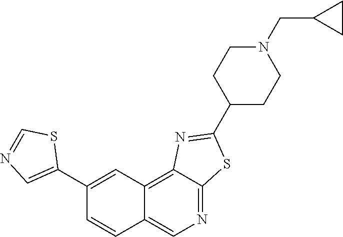 [SMILES: c1ncc(-c2ccc3cnc4sc(C5CCN(CC6CC6)CC5)nc4c3c2)s1] |
| 87 | 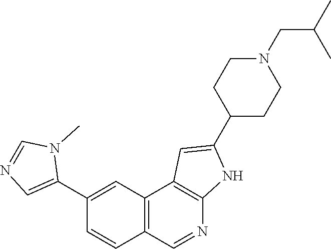 [SMILES: CC(C)CN1CCC(c2cc3c(ncc4ccc(-c5cncn5C)cc43)[nH]2)CC1] |
| 88 | 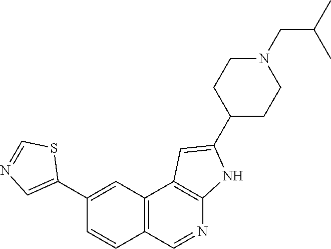 [SMILES: CC(C)CN1CCC(c2cc3c(ncc4ccc(-c5cncs5)cc43)[nH]2)CC1] |
| 89 | 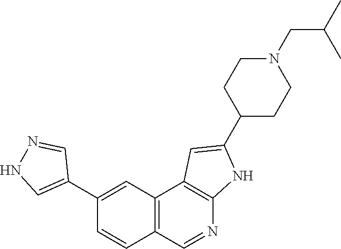 [SMILES: CC(C)CN1CCC(c2cc3c(ncc4ccc(-c5cn[nH]c5)cc43)[nH]2)CC1] |
| 95 | 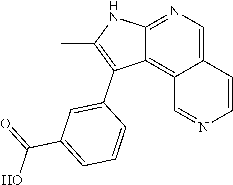 [SMILES: Cc1[nH]c2ncc3ccncc3c2c1-c1cccc(C(=O)O)c1] |

### Compounds of Formula (II)

The substituent groups used in this section (e.g., R¹, R², and the like) refer solely to the groups in Formula (II).

Some embodiments provide a compound of Formula (II-0):

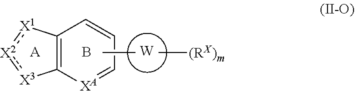

or a pharmaceutically acceptable salt thereof, wherein:
   each dashed line represents a single bond or a double bond;
   Rings A and B are aromatic;
   Ring W is a 9 membered heteroaryl, a 9 membered heterocyclyl, or a 9 membered cycloalkyl;
   each R^{X} is independently selected from halogen, cyano, hydroxyl, C1-C6 alkyl, C1-C6 haloalkyl, C1-C6 alkoxy, C1-C6 haloalkoxy, 3-6 membered heterocyclyl optionally substituted with C1-C6 alkyl or halogen, and C3-C6 cycloalkyl;
   X¹ is CH, S, N, or NR^{A};
   X² is N, CH, or CR²;
   X³ is N, NR^{B}, O, CR³, or CH;
   X⁴ is CH or N;
   R² is benzyl or

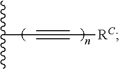

R³ is C1-C6 alkyl, C1-C6 haloalkyl, C1-C6 alkoxy, or C1-C6 haloalkoxy;
R^{A} is C1-C6 alkyl or C3-C6 cycloalkyl;
R^{B} is hydrogen, C1-C6 alkyl, or C3-C6 cycloalkyl;
R^{C} is 3-6 membered heterocyclyl optionally substituted with C1-C6 alkyl, halogen, or hydroxy;
m is 0, 1, or 2; and
n is 0 or 1.

Some embodiments provide a compound of Formula (II):

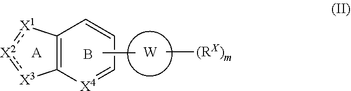

or a pharmaceutically acceptable salt thereof, wherein:
   each dashed line represents a single bond or a double bond;
   Rings A and B are aromatic;
   Ring W is a 9 membered heteroaryl, a 9 membered heterocyclyl, or a 9 membered cycloalkyl;
   each R^{X} is independently selected from C1-C6 alkyl, 3-6 membered heterocyclyl optionally substituted with C1-C6 alkyl, and C3-C6 cycloalkyl;
   X¹ is CH, S, N, or NR^{A};
   X² is N, CH, or CR²;
   X³ is N, NR^{B}, O, CR³, or CH;
   X⁴ is CH or N;
   R² is benzyl or n

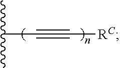

R³ is C1-C6 alkyl;
R^{A} is C1-C6 alkyl or C3-C6 cycloalkyl;
R^{B} is hydrogen or C3-C6 cycloalkyl;
R^{C} is 3-6 membered heterocyclyl optionally substituted with C1-C6 alkyl;
m is 0, 1, or 2; and
n is 0 or 1.

In some embodiments, the dashed line between X¹ and X² represents a single bond and the dashed line between X² and X³ represents a double bond.

In some embodiments, the dashed line between X¹ and X² represents a double bond and the dashed line between X² and X³ represents a single bond.

In some embodiments, Ring W is a 9 membered heteroaryl. In some embodiments, Ring W is pyrazolo[1,5-a]pyridinyl, pyrazolo[1,5-a]pyrimidinyl, 1H-pyrrolo[2,3-b]pyridinyl, 1H-pyrrolo[2,3-c]pyridinyl, thiazolo[4,5-c]pyridinyl, 1,3-dihydro-2H-imidazo[4,5-b]pyridin-2-only, indazolyl, or imidazo[1,2-a]pyrazine.

In some embodiments, Ring W is

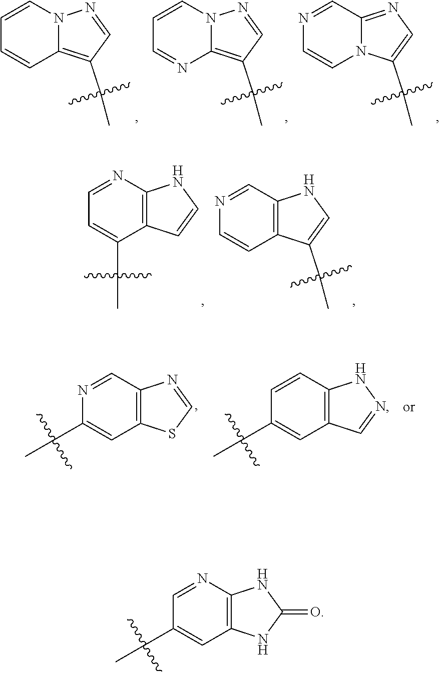

In some embodiments, Ring W is a 9 membered heterocyclyl. In some embodiments, Ring W is methylenedioxyphenyl. In some embodiments, Ring W is

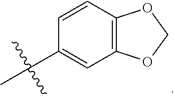 [SMILES: *c1ccc2c(c1)OCO2]

In some embodiments, Ring W is a 9 membered cycloalkyl.

In some embodiments, m is 0. In some embodiments, m is 1. In some embodiments, m is 2.

In some embodiments, at least one R^{X} is C1-C6 alkyl. In some embodiments, at least one R^{X} is methyl.

In some embodiments, at least one R^{X} is 3-6 membered heterocyclyl optionally substituted with C1-C6 alkyl. In some embodiments, at least one R^{X} is 3-6 membered heterocyclyl substituted with C1-C6 alkyl. In some embodiments, at least one R^{X} is 3-6 membered heterocyclyl substituted with methyl. In some embodiments, at least one R^{X} is 3-6 membered heterocyclyl substituted with ethyl. In some embodiments, at least one R^{X} is 3-6 membered heterocyclyl substituted with isobutyl. In some embodiments, at least one R^{X} is unsubstituted 3-6 membered heterocyclyl.

In some embodiments, the R^{X} heteroaryl is a 5-6 membered heteroaryl. In some embodiments, the R^{X} heteroaryl is a 5 membered heteroaryl. In some embodiments, the R^{X} heteroaryl is a 6 membered heteroaryl. In some embodiments, the R^{X} heteroaryl is a pyridonyl.

In some embodiments, at least one R^{X} is C3-C6 cycloalkyl. In some embodiments, at least one R^{X} is C3-C5 cycloalkyl. In some embodiments, at least one R^{X} is C3-C4 cycloalkyl.

In some embodiments, at least one R^{X} is cyclobutyl.

In some embodiments, X¹ is CH. In some embodiments, X¹ is S. In some embodiments, X¹ is N. In some embodiments, X¹ is NR^{A}.

In some embodiments, R^{A} is C1-C6 alkyl. In some embodiments, R^{A} is C1-C3 alkyl.

In some embodiments, R^{A} is methyl. In some embodiments, R^{A} is ethyl. In some embodiments, R^{A} is isopropyl.

In some embodiments, R^{A} is C3-C6 cycloalkyl. In some embodiments, R^{A} is C3-C4 cycloalkyl. In some embodiments, R^{A} is cyclopropyl. In some embodiments, R^{A} is cyclobutyl.

In some embodiments, X² is N. In some embodiments, X² is CH. In some embodiments, X² is CR².

In some embodiments, R² is benzyl. In some embodiments, R² is

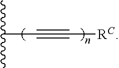

In some embodiments, n is 0. In some embodiments, n is 1.

In some embodiments, R^{C} is 3-6 membered heterocyclyl optionally substituted with C1-C6 alkyl. In some embodiments, R^{C} is 3-6 membered heterocyclyl substituted with C1-C6 alkyl. In some embodiments, R^{C} is 3-6 membered heterocyclyl substituted with methyl. In some embodiments, R^{C} is 3-6 membered heterocyclyl substituted with isobutyl. In some embodiments, R^{C} is unsubstituted 3-6 membered heterocyclyl. In some embodiments, the R^{C} heterocyclyl is a piperidinyl. In some embodiments, the R^{C} heterocyclyl is a 1-piperidinyl.

In some embodiments, X³ is N. In some embodiments, X³ is NR^{B}.

In some embodiments, R^{B} is hydrogen. In some embodiments, R^{B} is C3-C6 cycloalkyl.

In some embodiments, R^{B} is C3-C4 cycloalkyl. In some embodiments, R^{B} is cyclopropyl. In some embodiments, R^{B} is cyclobutyl.

In some embodiments, X³ is 0. In some embodiments, X³ is CH. In some embodiments, X³ is CR³.

In some embodiments, R³ is C1-C6 alkyl. In some embodiments, R³ is methyl.

In some embodiments, X⁴ is CH. In some embodiments, X⁴ is N.

In some embodiments, the compound is a compound of Formula (II-A):

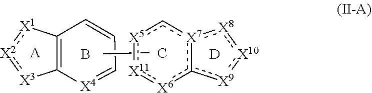

wherein:
   X¹, X², X³, and X⁴ are as defined in claim 1;
   Ring C is aromatic;
   X⁵ is CH, CR⁵, or N;
   X⁶ is CH or N;
   X⁷ is N or C;
   X⁸ is N, O, CH, or NR^{D};
   X⁹ is CH, NH, N, O, or S;
   X¹⁰ is CH, CH₂, C(═O), or N;
   X¹¹ is CH or N;
   R⁵ is 3-6 membered heterocyclyl optionally substituted with C1-C6 alkyl; and
   R^{D} is C1-C6 alkyl or C3-C6 cycloalkyl.

In some embodiments, the compound is a compound of Formula (II-A-i):

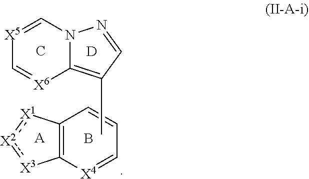

In some embodiments, the compound is a compound of Formula (II-A-ii):

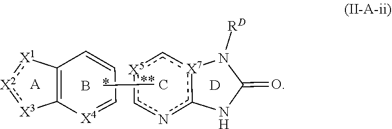

In some embodiments, the compound is a compound of Formula (II-A-iii):

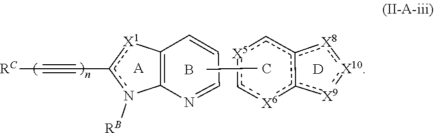

In some embodiments, the compound is a compound of Formula (I-A-iv):

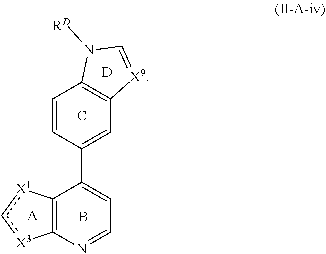

In some embodiments, the dashed line between X¹ and X² represents a single bond and the dashed line between X² and X³ represents a double bond.

In some embodiments, the dashed line between X¹ and X² represents a double bond and the dashed line between X² and X³ represents a single bond.

In some embodiments, X¹ is CH.

In some embodiments, X⁵ is N. In some embodiments, X⁵ is CH. In some embodiments, X⁵ is CR⁵.

In some embodiments, R⁵ is 3-6 membered heterocyclyl optionally substituted with C1-C6 alkyl. In some embodiments, R⁵ is 3-6 membered heterocyclyl substituted with C1-C6 alkyl. In some embodiments, R⁵ is 3-6 membered heterocyclyl substituted with methyl. In some embodiments, R⁵ is 3-6 membered heterocyclyl substituted with ethyl. In some embodiments, R⁵ is 3-6 membered heterocyclyl substituted with isobutyl. In some embodiments, R⁵ is unsubstituted 3-6 membered heterocyclyl.

In some embodiments, the R⁵ heteroaryl is a 5-6 membered heteroaryl. In some embodiments, the R⁵ heteroaryl is a 5 membered heteroaryl. In some embodiments, the R⁵ heteroaryl is a 6 membered heteroaryl. In some embodiments, the R⁵ heteroaryl is a pyridonyl.

In some embodiments, X⁶ is CH. In some embodiments, X⁶ is N.

In some embodiments, X⁷ is N. In some embodiments, X⁷ is C.

In some embodiments, X⁸ is N. In some embodiments, X⁸ is 0. In some embodiments, X⁸ is CH. In some embodiments, X⁸ is NR^{D}.

In some embodiments, R^{D} is C1-C6 alkyl. In some embodiments, R^{D} is methyl. In some embodiments, R^{D} is C3-C6 cycloalkyl. In some embodiments, R^{D} is C3-C5 cycloalkyl. In some embodiments, R^{D} is C3-C4 cycloalkyl. In some embodiments, R^{D} is cyclobutyl.

In some embodiments, X⁹ is CH. In some embodiments, X⁹ is NH. In some embodiments, X⁹ is N. In some embodiments, X⁹ is O. In some embodiments, X⁹ is S.

In some embodiments, X¹⁰ is CH. In some embodiments, X¹⁰ is CH₂. In some embodiments, X¹⁰ is C(═O). In some embodiments, X¹⁰ is N.

In some embodiments, X¹¹ is CH. In some embodiments, X¹¹ is N.

In some embodiments, the compound of Formula (II) is selected from a compound in Table 2, or a pharmaceutically acceptable salt thereof.

TABLE 2
Exemplary Compounds of Formula (II)

| Compound | |
|---|---|
| No. | Structure |
| 1 | 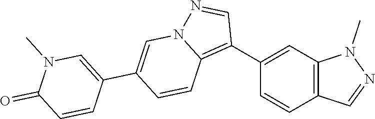 [SMILES: Cn1cc(-c2ccc3c(-c4ccc5cnn(C)c5c4)cnn3c2)ccc1=O] |
| 2 | 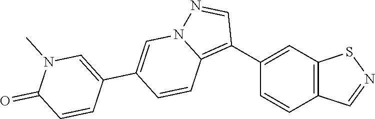 [SMILES: Cn1cc(-c2ccc3c(-c4ccc5cnsc5c4)cnn3c2)ccc1=O] |
| 3 | 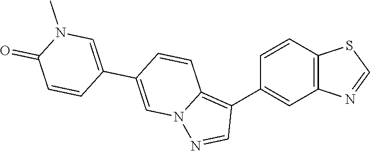 [SMILES: Cn1cc(-c2ccc3c(-c4ccc5scnc5c4)cnn3c2)ccc1=O] |
| 4 | 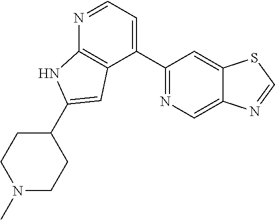 [SMILES: CN1CCC(c2cc3c(-c4cc5scnc5cn4)ccnc3[nH]2)CC1] |
| 5 | 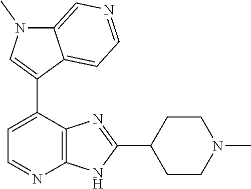 |
| 6 | 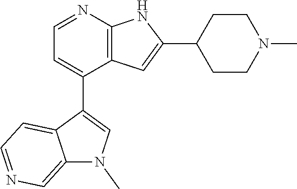 [SMILES: CN1CCC(c2cc3c(-c4cn(C)c5cnccc45)ccnc3[nH]2)CC1] |
| 7 | 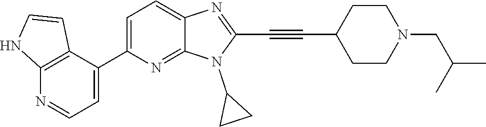 |
| 8 | 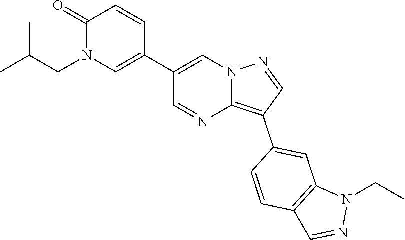 [SMILES: CCn1ncc2ccc(-c3cnn4cc(-c5ccc(=O)n(CC(C)C)c5)cnc34)cc21] |
| 9 | 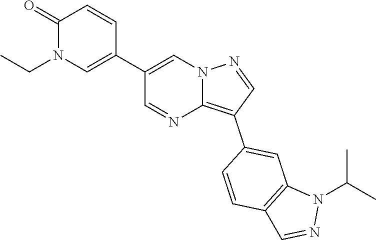 [SMILES: CCn1cc(-c2cnc3c(-c4ccc5cnn(C(C)C)c5c4)cnn3c2)ccc1=O] |

### Compounds of Formula (III)

The substituent groups used in this section (e.g., R¹, R², and the like) refer solely to the groups in Formula (III).

Some embodiments provide a compound of Formula (III):

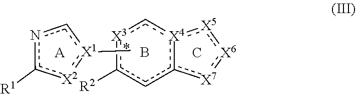

or a pharmaceutically acceptable salt thereof, wherein:
   each dashed line represents a single bond or a double bond;
   Rings A and B are aromatic;
   Ring C is aromatic or partially saturated;
   X¹ is C or N;
   X² is CH, O, or S;
   X³ is CH or N;
   X⁴ is C or N;
   X⁵ is CH, C(═O), O, S, NH, or NR^{A};
   X⁶ is CH, N, or O;
   X⁷ is CH, CR⁷, CH₂, CR^{B}R^{C}, or N;
   R¹ is hydrogen or —XR^{D};
   R² is hydrogen, —NHC(═O)-3-6 membered heterocyclyl optionally substituted with C1-C6 haloalkyl, or joins with the bond denoted with *;
   R⁷ is C1-C6 alkyl optionally substituted with a 3-6 membered heterocyclyl optionally substituted with C(═O)OH;
   R^{A} is -(3-6 membered heterocyclyl)ₘ-(C1-C6 alkyl)ₙ;
   R^{B} and R^{C} are independently hydrogen or C1-C6 alkyl;
X is ethynylene, —NHC(═O)—, or —NHC(═O)OCH₂—;
   R^{D} is 3-6 membered heterocyclyl optionally substituted with C1-C6 alkyl, 5-6 membered heteroaryl optionally substituted with C1-C6 alkyl, or C6-C10 aryl optionally substituted with C1-C6 alkyl;
   m is 0 or 1; and
   n is 0, 1, or 2;
   provided that when n is 2, m is 1.

In some embodiments, R¹ is hydrogen.

In some embodiments, R¹ is —XR^{D}.

In some embodiments, X is ethynylene. In some embodiments, X is —NHC(═O)—. In some embodiments, X is —NHC(═O)OCH₂—.

In some embodiments, R^{D} is 3-6 membered heterocyclyl optionally substituted with C1-C6 alkyl. In some embodiments, R^{D} is 3-6 membered heterocyclyl substituted with C1-C6 alkyl. In some embodiments, R^{D} is 3-6 membered heterocyclyl optionally substituted with methyl. In some embodiments, R^{D} is 3-6 membered heterocyclyl substituted with methyl. In some embodiments, R^{D} is unsubstituted 3-6 membered heterocyclyl. In some embodiments, the R^{D} 3-6 membered heterocyclyl is 5-6 membered heterocyclyl. In some embodiments, the R^{D} 3-6 membered heterocyclyl is piperidinyl.

In some embodiments, R^{D} is 5-6 membered heteroaryl optionally substituted with C1-C6 alkyl. In some embodiments, R^{D} is 5-6 membered heteroaryl substituted with C1-C6 alkyl. In some embodiments, R^{D} is 5-6 membered heteroaryl optionally substituted with methyl. In some embodiments, R^{D} is 5-6 membered heteroaryl substituted with methyl. In some embodiments, R^{D} is unsubstituted 5-6 membered heteroaryl. In some embodiments, the R^{D} 5-6 membered heteroaryl is 6 membered heteroaryl. In some embodiments, the R^{D} 5-6 membered heteroaryl is pyridonyl.

In some embodiments, R^{D} is C6-C10 aryl optionally substituted with C1-C6 alkyl. In some embodiments, R^{D} is C6-C10 aryl substituted with C1-C6 alkyl. In some embodiments, R^{D} is C6-C10 aryl optionally substituted with methyl. In some embodiments, R^{D} is C6-C1O aryl substituted with methyl. In some embodiments, R^{D} is unsubstituted C6-C10 aryl. In some embodiments, the R^{D} C6-C10 aryl is phenyl.

In some embodiments, R² is hydrogen. In some embodiments, R² is —NHC(═O)-3-6 membered heterocyclyl optionally substituted with C1-C6 haloalkyl. In some embodiments, R² is —NHC(═O)-3-6 membered heterocyclyl optionally substituted with fluoroethyl. In some embodiments, R² is —NHC(═O)-3-6 membered heterocyclyl substituted with C1-C6 haloalkyl.

In some embodiments, R² is —NHC(═O)-3-6 membered heterocyclyl substituted with fluoroethyl. In some embodiments, R² is unsubstituted —NHC(═O)-3-6 membered heterocyclyl. In some embodiments, the R² —NHC(═O)-3-6 membered heterocyclyl is —NHC(═O)-piperidinyl. In some embodiments, R² joins with the bond denoted with *.

In some embodiments, Ring A is attached to the position of Ring B ortho to X³ and X⁴.

In some embodiments, Ring A is attached to the position of Ring B ortho to R² and meta to X³ and X⁴.

In some embodiments, X¹ is C. In some embodiments, X¹ is N.

In some embodiments, X² is CH. In some embodiments, X² is O. In some embodiments, X² is S.

In some embodiments, X³ is CH. In some embodiments, the X³ CH is substituted with Ring A. In some embodiments, X³ is N.

In some embodiments, X⁴ is C. In some embodiments, X⁴ is N. In some embodiments, X⁵ is CH.

In some embodiments, the X⁵ CH is substituted with Ring A. In some embodiments, X⁵ is C(═O). In some embodiments, X⁵ is O. In some embodiments, X⁵ is S. In some embodiments, X⁵ is NH. In some embodiments, X⁵ is NR^{A}.

In some embodiments, R^{A} is -(3-6 membered heterocyclyl)ₘ-(C1-C6 alkyl)ₙ. In some embodiments, R^{A} is -(3-6 membered heterocyclyl)ₘ-(methyl)ₙ. In some embodiments, R^{A} is -(5-6 membered heterocyclyl)ₘ-(methyl)ₙ. In some embodiments, R^{A} is -(piperidinyl)ₘ-(methyl)ₙ.

In some embodiments, m is O. In some embodiments, m is 1.

In some embodiments, n is O. In some embodiments, n is 1.

In some embodiments, n is 2 and m is 1.

In some embodiments, X⁶ is CH. In some embodiments, the X⁶ CH is substituted with Ring A. In some embodiments, X⁶ is N. In some embodiments, X⁶ is O.

In some embodiments, X⁷ is CH. In some embodiments, the X⁷ CH is substituted with Ring A. In some embodiments, X⁷ is CR⁷.

In some embodiments, R⁷ is C1-C6 alkyl optionally substituted with a 3-6 membered heterocyclyl optionally substituted with C(═O)OH. In some embodiments, R⁷ is C1-C6 alkyl optionally substituted with a 3-6 membered heterocyclyl substituted with C(═O)OH. In some embodiments, R⁷ is C1-C6 alkyl substituted with a 3-6 membered heterocyclyl optionally substituted with C(═O)OH. In some embodiments, R⁷ is C1-C6 alkyl substituted with a 3-6 membered heterocyclyl substituted with C(═O)OH. In some embodiments, R⁷ is C1-C6 alkyl substituted with an unsubstituted 3-6 membered heterocyclyl. In some embodiments, the R⁷ 3-6 membered heterocyclyl is 5-6 membered heterocyclyl. In some embodiments, the R⁷ 3-6 membered heterocyclyl is pyrrolidinyl.

In some embodiments, R⁷ is unsubstituted C1-C6 alkyl. In some embodiments, the R⁷ C1-C6 alkyl is methyl. In some embodiments, X⁷ is CH₂.

In some embodiments, X⁷ is CR^{B}R^{C}. In some embodiments, R^{B} and R^{C} are hydrogen. In some embodiments, R^{B} is C1-C6 alkyl and R^{C} is hydrogen. In some embodiments, R^{B} is methyl and R^{C} is hydrogen. In some embodiments, R^{B} is hydrogen and R^{C} is C1-C6 alkyl. In some embodiments, R^{B} is hydrogen and R^{C} is methyl. In some embodiments, R^{B} and R^{C} are independently selected C1-C6 alkyl. In some embodiments, R^{B} and R^{C} are methyl. In some embodiments, X⁷ is N.

In some embodiments, Ring A is

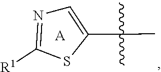

wherein the bond crossed by the wavy line is connected to Ring B or Ring C.

In some embodiments, Ring A is

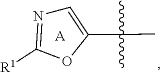

wherein the bond crossed by the wavy line is connected to Ring B or Ring C.

In some embodiments, Ring A is

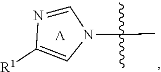

wherein the bond crossed by the wavy line is connected to Ring B or Ring C.

In some embodiments, Rings B and C are collectively

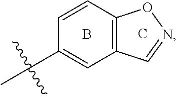

wherein the bond crossed by the wavy line is connected to Ring A.

In some embodiments, Rings B and C are collectively

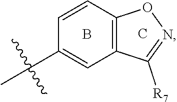

wherein the bond crossed by the wavy line is connected to Ring A.

In some embodiments, Rings B and C are collectively

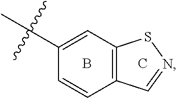

wherein the bond crossed by the wavy line is connected to Ring A.

In some embodiments, Rings B and C are collectively

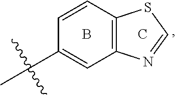

wherein the bond crossed by the wavy line is connected to Ring A.

In some embodiments, Rings B and C are collectively

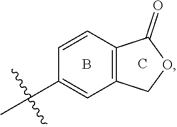

wherein the bond crossed by the wavy line is connected to Ring A.

In some embodiments, Rings B and C are collectively

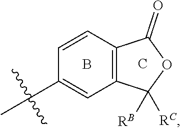

wherein the bond crossed by the wavy line is connected to Ring A.

In some embodiments, Rings B and C are collectively

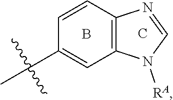

wherein the bond crossed by the wavy line is connected to Ring A.

In some embodiments, Rings B and C are collectively

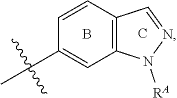

wherein the bond crossed by the wavy line is connected to Ring A.

In some embodiments, Rings B and C are collectively

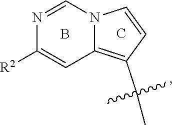

wherein the bond crossed by the wavy line is connected to Ring A.

In some embodiments, the compound of Formula (III) is a compound of Formula (III-A):

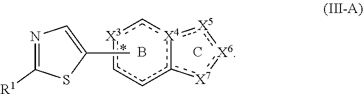

In some embodiments, the compound of Formula (III) is a compound of Formula (III-B):

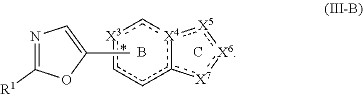

In some embodiments, the compound of Formula (III) is a compound of Formula (III-C):

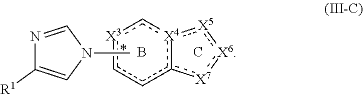

In some embodiments, the compound of Formula (III) is a compound of Formula (III-D):

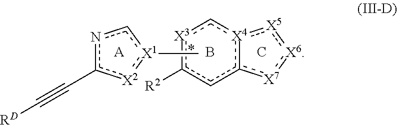

In some embodiments, the compound of Formula (III) is a compound of Formula (III-E):

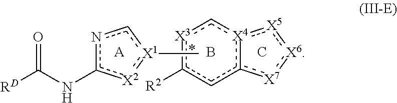

In some embodiments, the compound of Formula (III) is a compound of Formula (III-F):

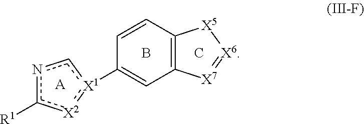

In some embodiments, the compound of Formula (III) is selected from a compound in Table 3, or a pharmaceutically acceptable salt thereof.

TABLE 3
Exemplary Compounds of Formula (III)

| Com- | |
|---|---|
| pound | |
| No. | Structure |
| 1 | 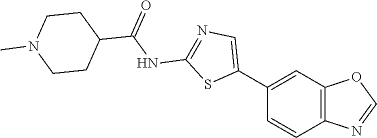 [SMILES: CN1CCC(C(=O)Nc2ncc(-c3ccc4ncoc4c3)s2)CC1] |
| 2 | 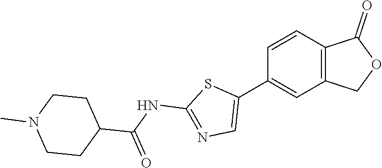 [SMILES: CN1CCC(C(=O)Nc2ncc(-c3ccc4c(c3)COC4=O)s2)CC1] |
| 3 | 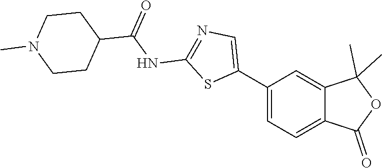 |
| 4 | 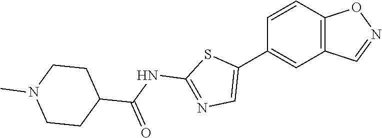 [SMILES: CN1CCC(C(=O)Nc2ncc(-c3ccc4oncc4c3)s2)CC1] |
| 5 | 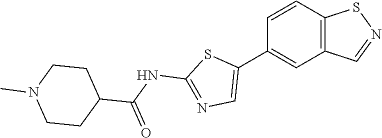 [SMILES: CN1CCC(C(=O)Nc2ncc(-c3ccc4sncc4c3)s2)CC1] |
| 6 | 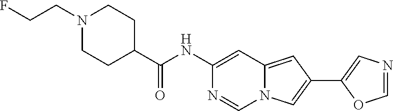 [SMILES: O=C(Nc1cc2cc(-c3cnco3)cn2cn1)C1CCN(CCF)CC1] |
| 7 | 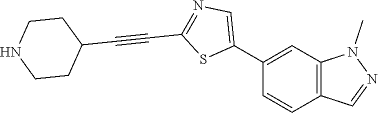 [SMILES: Cn1ncc2ccc(-c3cnc(C#CC4CCNCC4)s3)cc21] |
| 8 | 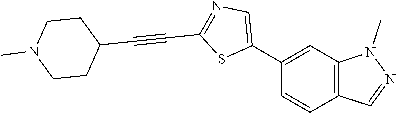 [SMILES: CN1CCC(C#Cc2ncc(-c3ccc4cnn(C)c4c3)s2)CC1] |
| 9 | 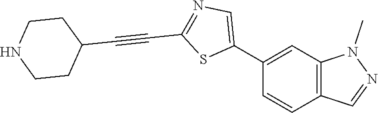 [SMILES: Cn1ncc2ccc(-c3cnc(C#CC4CCNCC4)s3)cc21] |
| 10 | 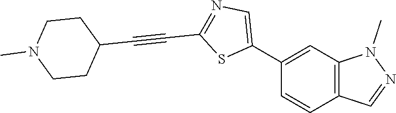 [SMILES: CN1CCC(C#Cc2ncc(-c3ccc4cnn(C)c4c3)s2)CC1] |

### Compounds of Formula (IV)

The substituent groups used in this section (e.g., R¹, R², and the like) refer solely to the groups in Formula (IV).

Some embodiments provide a compound of Formula (IV):

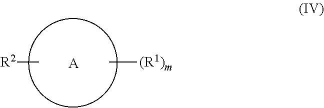

or a pharmaceutically acceptable salt thereof, wherein:
   Ring A is a 12-14 membered fused tricyclic heterocyclyl comprising 2-5 nitrogen atoms or a 12-14 membered fused tricyclic heteroaryl comprising 2-5 nitrogen atoms;
   R¹ is cyano, C1-C6 alkyl, —NHC(═O)(C1-C6 alkylene)ₙR^{A}, -Q-phenyl optionally substituted with 1-2 substituents independently selected from halogen, hydroxyl, and C1-C6 alkoxy; C3-C6 cycloalkyl optionally substituted with hydroxyl, —(C1-C6 alkylene)ₚ-5-10 membered heteroaryl, or 5-10 membered heterocyclyl;
   R² is hydrogen, cyano, C1-C6 alkyl, —C(═O)—C1-C6 alkyl, —(SO₂)C1-C6 alkyl, —CO₂R^{B}, C1-C6 alkoxy optionally substituted with —NR^{C}R^{D};
   R^{A} is 4-6 membered heterocyclyl optionally substituted with C1-C6 alkyl, or 5-10 membered heteroaryl optionally substituted with C1-C6 alkoxy or C1-C6 alkyl;
   R^{B}, R^{C}, and R^{D} are independently hydrogen or C1-C6 alkyl; Q is a bond or O;
   m is 0 or 1;
   n is 0 or 1; and
   p is 0 or 1.

In some embodiments, R¹ is cyano.

In some embodiments, R¹ is C1-C6 alkyl. In some embodiments, R¹ is methyl.

In some embodiments, R¹ is —NHC(═O)(C1-C6 alkylene)ₙR^{A}. In some embodiments, R¹ is —NHC(═O)(C1-C4 alkylene)ₙR^{A}. In some embodiments, R¹ is —NHC(═O)(C1-C2 alkylene)ₙR^{A}.

In some embodiments, R^{A} is 4-6 membered heterocyclyl optionally substituted with C1-C6 alkyl. In some embodiments, R^{A} is oxetanyl, azetidinyl, pyrrolidinyl, pyrrolidinonyl, imidazolidinyl, piperidinyl, piperazinyl, or morpholinyl; each optionally substituted with C1-C6 alkyl. In some embodiments, R^{A} is 4-6 membered heterocyclyl substituted with C1-C6 alkyl. In some embodiments, R^{A} is unsubstituted 4-6 membered heterocyclyl.

In some embodiments, R^{A} is 5-10 membered heteroaryl optionally substituted with C1-C6 alkoxy or C1-C6 alkyl. In some embodiments, R^{A} is 5-10 membered heteroaryl substituted with C1-C6 alkoxy. In some embodiments, R^{A} is 5-10 membered heteroaryl substituted with C1-C6 alkyl. In some embodiments, R^{A} is 5-6 membered heteroaryl optionally substituted with C1-C6 alkoxy or C1-C6 alkyl. In some embodiments, R^{A} is pyrrolyl, imidazolyl, oxazolyl, thiazolyl, pyridinyl, pyrimidinyl, or pyrazinyl; each optionally substituted with C1-C6 alkoxy or C1-C6 alkyl.

In some embodiments, R^{A} is 5-6 membered heteroaryl substituted with C1-C6 alkoxy. In some embodiments, R^{A} is 5-6 membered heteroaryl substituted with C1-C6 alkyl. In some embodiments, R^{A} is unsubstituted 5-6 membered heteroaryl.

In some embodiments, n is O. In some embodiments, n is 1.

In some embodiments, R¹ is -Q-phenyl optionally substituted with 1-2 substituents independently selected from halogen, hydroxyl, and C1-C6 alkoxy. In some embodiments, R¹ is -Q-phenyl substituted with 1-2 substituents independently selected from halogen, hydroxyl, and C1-C6 alkoxy. In some embodiments, R¹ is -Q-phenyl substituted with one or two independently selected halogen. In some embodiments, R¹ is -Q-phenyl substituted with one halogen. In some embodiments, R¹ is -Q-phenyl substituted with one hydroxyl. In some embodiments, R¹ is -Q-phenyl substituted with one or two independently selected C1-C6 alkoxy. In some embodiments, R¹ is -Q-phenyl substituted with one C1-C6 alkoxy. In some embodiments, R¹ is -Q-phenyl substituted with one halogen and one C1-C6 alkoxy. In some embodiments, R¹ is -Q-unsubstituted phenyl. In some embodiments, Q is a bond. In some embodiments, Q is O.

In some embodiments, R¹ is C3-C6 cycloalkyl optionally substituted with hydroxyl. In some embodiments, R¹ is C3-C6 cycloalkyl substituted with hydroxyl. In some embodiments, R¹ is unsubstituted C3-C6 cycloalkyl.

In some embodiments, R¹ is —(C1-C6 alkylene)ₚ-5-10 membered heteroaryl. In some embodiments, R¹ is —(C1-C6 alkylene)ₚ-5-6 membered heteroaryl. In some embodiments, R¹ is —(C1-C6 alkylene)ₚ-pyrrolyl, —(C1-C6 alkylene)ₚ-imidazolyl, —(C1-C6 alkylene)ₚ-oxazolyl, —(C1-C6 alkylene)ₚ-thiazolyl, —(C1-C6 alkylene)ₚ-pyridinyl, —(C1-C6 alkylene)ₚ-pyrimidinyl, or —(C1-C6 alkylene)ₚ-pyrazinyl. In some embodiments, R¹ is —(C1-C2 alkylene)ₚ-5-6 membered heteroaryl. In some embodiments, R¹ is —(C1-C2 alkylene)ₚ-pyrrolyl, —(C1-C2 alkylene)ₚ-imidazolyl, —(C1-C2 alkylene)ₚ-oxazolyl, —(C1-C2 alkylene)ₚ-thiazolyl, —(C1-C2 alkylene)ₚ-pyridinyl, —(C1-C2 alkylene)ₚ-pyrimidinyl, or —(C1-C2 alkylene)ₚ-pyrazinyl.

In some embodiments, p is 0. In some embodiments, p is 1.

In some embodiments, R¹ is a 5-10 membered heterocyclyl. In some embodiments, R¹ is a 5-6 membered heterocyclyl. In some embodiments, R¹ is tetrahydropyran or dihydropyran. In some embodiments, R¹ is a fused bicyclic 9-10 membered heterocyclyl. In some embodiments, R¹ is [1,3]dioxolo[4,5-b]pyridine, benzo[d][1,3]dioxole, 2,3-dihydrofuro[2,3-b]pyridine, 2,3-dihydrobenzofuran, or 2,3-dihydrofuro[3,2-b]pyridine.

In some embodiments, R² is hydrogen. In some embodiments, R² is cyano. In some embodiments, R² is C1-C6 alkyl. In some embodiments, R² is methyl.

In some embodiments, R² is —C(═O)—C1-C6 alkyl. In some embodiments, R² is —C(═O)CH₃.

In some embodiments, R² is —(SO₂)C1-C6 alkyl. In some embodiments, R² is —(SO₂)CH₃.

In some embodiments, R² is —CO₂R^{B}.

In some embodiments, R^{B} is C1-C6 alkyl. In some embodiments, R² is —CO₂CH₃. In some embodiments, R^{B} is hydrogen.

In some embodiments, R² is C1-C6 alkoxy optionally substituted with —NR^{C}R^{D}. In some embodiments, R² is C1-C6 alkoxy substituted with —NR^{C}R^{D}. In some embodiments, R² is ethoxy substituted with —NR^{C}R^{D}. In some embodiments, R² is unsubstituted C1-C6 alkoxy.

In some embodiments, R² is methoxy.

In some embodiments, R^{C} is C1-C6 alkyl. In some embodiments, R^{C} is methyl. In some embodiments, R^{C} is hydrogen, In some embodiments, R^{D} is C1-C6 alkyl. In some embodiments, R^{D} is methyl. In some embodiments, R^{D} is hydrogen. In some embodiments, R^{C} and R^{D} are the same. In some embodiments, R^{C} and R^{D} are different. In some embodiments, R^{C} and R^{D} are each hydrogen. In some embodiments, R^{C} and R^{D} are each methyl. In some embodiments, one of R^{C} and R^{D} is hydrogen and the other of R^{C} and R^{D} is C1-C6 alkyl.

In some embodiments, m is 1. In some embodiments, m is 0.

In some embodiments, Ring A is 12-14 membered fused tricyclic heterocyclyl comprising 2-4 nitrogen atoms.

In some embodiments, Ring A is 12-14 membered fused tricyclic heterocyclyl comprising 2-3 nitrogen atoms.

In some embodiments, Ring A is 12-14 membered fused tricyclic heterocyclyl comprising 2 nitrogen atoms.

In some embodiments, Ring A is 12-14 membered fused tricyclic heterocyclyl comprising one all-carbon 5-6 membered ring.

In some embodiments, Ring A is 12-14 membered fused tricyclic heterocyclyl further comprising one oxygen atom.

In some embodiments, Ring A is 12-14 membered fused tricyclic heteroaryl comprising 2-4 nitrogen atoms.

In some embodiments, Ring A is 12-14 membered fused tricyclic heteroaryl comprising 2-3 nitrogen atoms.

In some embodiments, Ring A is 12-14 membered fused tricyclic heteroaryl comprising 2 nitrogen atoms.

In some embodiments, Ring A is 12-14 membered fused tricyclic heteroaryl comprising one all-carbon 6 membered ring.

In some embodiments, Ring A is 6H-isochromeno[3,4-d]pyrimidine, 5,7-dihydro-2H-imidazo[4′,5′:4,5]benzo[1,2-d]oxazole-2,6(3H)-dione, 5,7-dihydroimidazo[4,5-f]indazol-6(1H)-one, oxazolo[4,5-g]isoquinolin-2(1H)-one; 1,7-dihydro-6H-oxazolo[5,4-f]indazol-6-one, 6,7,8,9-tetrahydro-3H-pyrrolo[2,3-c][2,7]naphthyridine, benzo[c][2,6]naphthyridine, 1,3,4,5-tetrahydrobenzo[c][1,7]naphthyridin-6(2H)-one, 3H-pyrazolo[3,4-c]quinolone, 2,3,4,7-tetrahydro-1H-pyrrolo[2,3-c][2,6]naphthyridine, or pyrrolo[1,2-a]quinoxalin-4(5H)-one.

In some embodiments, Ring A is 6H-isochromeno[3,4-d]pyrimidine. In some embodiments, Ring A is 5,7-dihydro-2H-imidazo[4′,5′:4,5]benzo[1,2-d]oxazole-2,6(3H)-dione. In some embodiments, Ring A is 5,7-dihydroimidazo[4,5-f]indazol-6(1H)-one. In some embodiments, Ring A is oxazolo[4,5-g]isoquinolin-2(1H)-one. In some embodiments, Ring A is 1,7-dihydro-6H-oxazolo[5,4-f]indazol-6-one. In some embodiments, Ring A is 6,7,8,9-tetrahydro-3H-pyrrolo[2,3-c][2,7]naphthyridine. In some embodiments, Ring A is benzo[c][2,6]naphthyridine. In some embodiments, Ring A is 1,3,4,5-tetrahydrobenzo[c][1,7]naphthyridin-6(2H)-one. In some embodiments, Ring A is 3H-pyrazolo[3,4-c]quinolone. In some embodiments, Ring A is 2,3,4,7-tetrahydro-1H-pyrrolo[2,3-c][2,6]naphthyridine. In some embodiments, Ring A is pyrrolo[1,2-a]quinoxalin-4(5H)-one.

In some embodiments, Ring A is

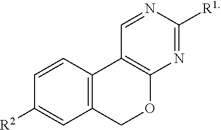

In some embodiments, Ring A is

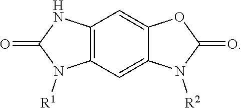

In some embodiments, Ring A is

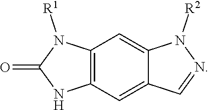

In some embodiments, Ring A is

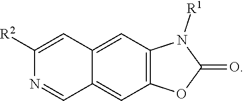

In some embodiments, Ring A is

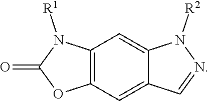

In some embodiments, Ring A is

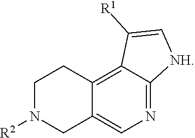

In some embodiments, Ring A is

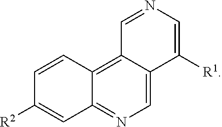

In some embodiments, Ring A is

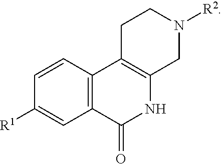

In some embodiments, Ring A is

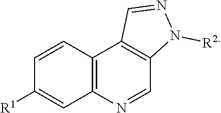

In some embodiments, Ring A is

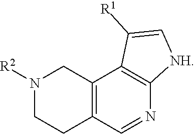

In some embodiments, Ring A is

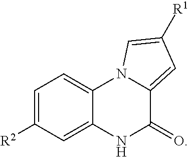

In some embodiments, the compound of Formula (IV) is selected from a compound in Table 4, or a pharmaceutically acceptable salt thereof.

TABLE 4
Exemplary Compounds of Formula (IV)

| Com- | |
|---|---|
| pound | |
| No. | Structure |
| 1 | 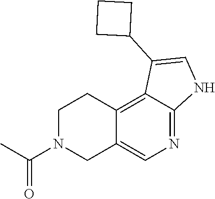 [SMILES: CC(=O)N1CCc2c(cnc3[nH]cc(C4CCC4)c23)C1] |
| 2 | 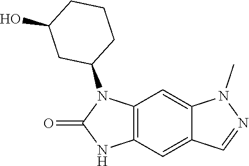 [SMILES: Cn1ncc2cc3[nH]c(=O)n([C@@H]4CCC[C@H](O)C4)c3cc21] |
| 3 | 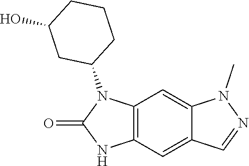 [SMILES: Cn1ncc2cc3[nH]c(=O)n([C@H]4CCC[C@@H](O)C4)c3cc21] |
| 4 | 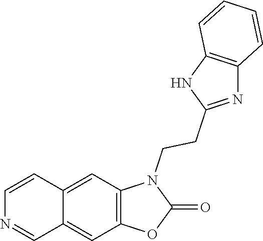 [SMILES: O=c1oc2cc3cnccc3cc2n1CCc1nc2ccccc2[nH]1] |
| 5 | 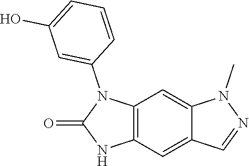 [SMILES: Cn1ncc2cc3[nH]c(=O)n(-c4cccc(O)c4)c3cc21] |
| 6 | 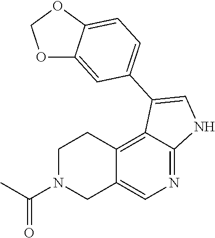 [SMILES: CC(=O)N1CCc2c(cnc3[nH]cc(-c4ccc5c(c4)OCO5)c23)C1] |
| 7 | 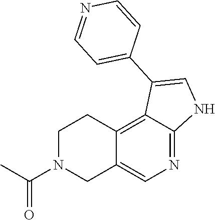 [SMILES: CC(=O)N1CCc2c(cnc3[nH]cc(-c4ccncc4)c23)C1] |
| 8 | 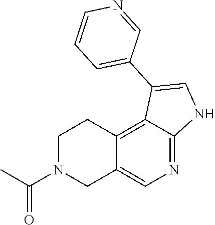 [SMILES: CC(=O)N1CCc2c(cnc3[nH]cc(-c4cccnc4)c23)C1] |
| 9 | 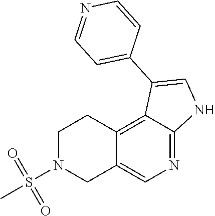 [SMILES: CS(=O)(=O)N1CCc2c(cnc3[nH]cc(-c4ccncc4)c23)C1] |
| 10 | 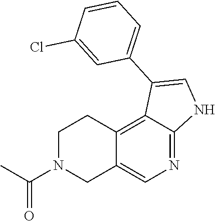 [SMILES: CC(=O)N1CCc2c(cnc3[nH]cc(-c4cccc(Cl)c4)c23)C1] |
| 11 | 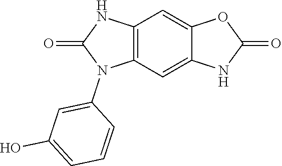 [SMILES: O=c1[nH]c2cc3c(cc2o1)[nH]c(=O)n3-c1cccc(O)c1] |
| 12 | 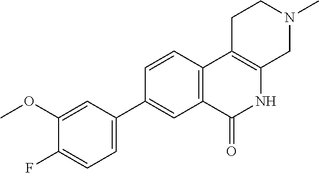 |
| 13 | 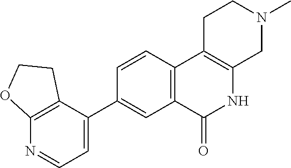 [SMILES: CN1CCc2c([nH]c(=O)c3cc(-c4ccnc5c4CCO5)ccc23)C1] |
| 14 | 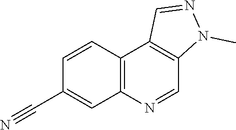 [SMILES: Cn1ncc2c3ccc(C#N)cc3ncc21] |
| 15 | 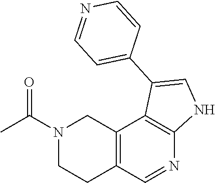 [SMILES: CC(=O)N1CCc2cnc3[nH]cc(-c4ccncc4)c3c2C1] |
| 16 | 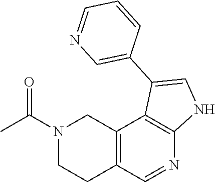 [SMILES: CC(=O)N1CCc2cnc3[nH]cc(-c4cccnc4)c3c2C1] |
| 17 | 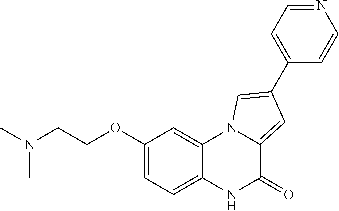 [SMILES: CN(C)CCOc1ccc2[nH]c(=O)c3cc(-c4ccncc4)cn3c2c1] |
| 18 | 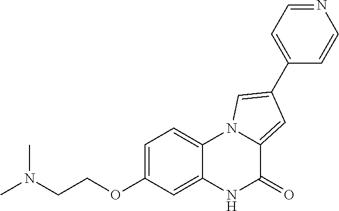 [SMILES: CN(C)CCOc1ccc2c(c1)[nH]c(=O)c1cc(-c3ccncc3)cn12] |
| 19 | 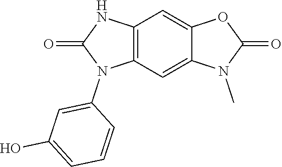 [SMILES: Cn1c(=O)oc2cc3[nH]c(=O)n(-c4cccc(O)c4)c3cc21] |
| 20 | 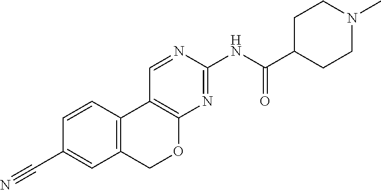 [SMILES: CN1CCC(C(=O)Nc2ncc3c(n2)OCc2cc(C#N)ccc2-3)CC1] |
| 21 | 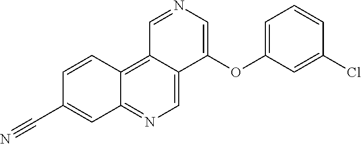 [SMILES: N#Cc1ccc2c(c1)ncc1c(Oc3cccc(Cl)c3)cncc12] |
| 22 | 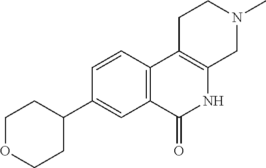 [SMILES: CN1CCc2c([nH]c(=O)c3cc(C4CCOCC4)ccc23)C1] |
| 23 | 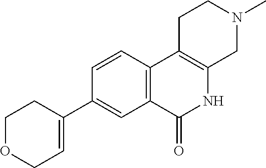 [SMILES: CN1CCc2c([nH]c(=O)c3cc(C4=CCOCC4)ccc23)C1] |
| 24 | 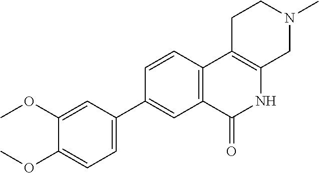 |
| 25 | 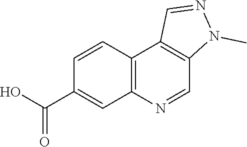 [SMILES: Cn1ncc2c3ccc(C(=O)O)cc3ncc21] |
| 26 | 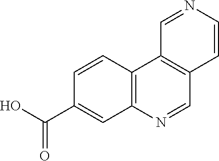 [SMILES: O=C(O)c1ccc2c(c1)ncc1ccncc12] |

### Pharmaceutical Compositions and Administration

### General

In some embodiments, the compounds described herein (e.g., compounds of Formula (I), (II), (III), (IV), and pharmaceutically acceptable salts of any of the foregoing, are administered as a pharmaceutical composition that includes the chemical compound and one or more pharmaceutically acceptable excipients. In some embodiments, the compound or a pharmaceutically acceptable salt thereof is a compound of Formula (I), or a pharmaceutically acceptable salt thereof. In some embodiments, the compound or a pharmaceutically acceptable salt thereof is a compound of Formula (II), or a pharmaceutically acceptable salt thereof. In some embodiments, the compound or a pharmaceutically acceptable salt thereof is a compound of Formula (III), or a pharmaceutically acceptable salt thereof. In some embodiments, the compound or a pharmaceutically acceptable salt thereof is a compound of Formula (IV), or a pharmaceutically acceptable salt thereof.

In some embodiments, the compounds can be administered in combination with one or more conventional pharmaceutical excipients as described herein. Dosage forms or compositions containing a chemical entity as described herein in the range of 0.005% to 100% with the balance made up from one or more pharmaceutically acceptable excipients may be prepared. The contemplated compositions may contain 0.001%-100% of a compound (or pharmaceutically acceptable salt thereof) provided herein, for example, from 0.1-95%, 75-85%, or 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

### Routes of Administration and Composition Components

In some embodiments, the compounds described herein or a pharmaceutical composition thereof can be administered to subject in need thereof by any accepted route of administration. Acceptable routes of administration include, but are not limited to, buccal, epidural, intracerebral, intradural, intramedullary, intrameningeal, intramuscular, intraspinal, intravascular, intravenous, nasal, oral, parenteral, peridural, respiratory (inhalation), subcutaneous, sublingual, submucosal, topical, transdermal, and transmucosal. In certain embodiments, a preferred route of administration is parenteral. In certain embodiments, a preferred route of administration is oral.

Compositions can be formulated for parenteral administration, e.g., formulated for injection via the intravenous, intramuscular, or sub-cutaneous routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

In other embodiments, the compounds described herein or a pharmaceutical composition thereof are suitable for local delivery to the digestive or GI tract by way of oral administration (e.g., solid or liquid dosage forms).

Solid dosage forms for oral administration include capsules, tablets, pills, powders, and granules. In such solid dosage forms, the compounds is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In some embodiments, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule).

Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds (i.e., excipients) include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

### Dosages

The dosages may be varied depending on the requirement of the patient, the severity of the condition being treating and the particular compound being employed. Determination of the proper dosage for a particular situation can be determined by one skilled in the medical arts. The total daily dosage may be divided and administered in portions throughout the day or by means providing continuous delivery.

In some embodiments, the compounds described herein are administered at a dosage of from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 1 mg/kg; from about 0.1 mg/kg to about 0.5 mg/kg).

### Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a therapeutic compound is administered to an individual for a period of time followed by a separate period of time. In another embodiment, a therapeutic compound is administered for a first period and a second period following the first period, with administration stopped during the second period, followed by a third period where administration of the therapeutic compound is started and then a fourth period following the third period where administration is stopped. In an aspect of this embodiment, the period of administration of a therapeutic compound followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

### Methods of Treatment

### Indications

This disclosure provides compounds of Formula (I), (II), (III), (IV), and pharmaceutically acceptable salts of any of the foregoing, that inhibit Dual specificity tyrosine-phosphorylation-regulated kinase 1A (DYRK1A). These compounds are useful for treating neurological disorders, e.g., DYRK1A-associated neurological disorders. In some embodiments, the compound or a pharmaceutically acceptable salt thereof is a compound of Formula (I), or a pharmaceutically acceptable salt thereof. In some embodiments, the compound or a pharmaceutically acceptable salt thereof is a compound of Formula (II), or a pharmaceutically acceptable salt thereof. In some embodiments, the compound or a pharmaceutically acceptable salt thereof is a compound of Formula (III), or a pharmaceutically acceptable salt thereof. In some embodiments, the compound or a pharmaceutically acceptable salt thereof is a compound of Formula (IV), or a pharmaceutically acceptable salt thereof. “Neurological disorder” refers to any disease or disorder of the nervous system and/or visual system. “Neurological disease” or “neurological disorder” are used interchangeably herein, and include diseases or disorders that involve the central nervous system (CNS; e.g., brain, brainstem and cerebellum), the peripheral nervous system (PNS; including cranial nerves), and the autonomic nervous system (parts of which are located in both the CNS and PNS), including both structural and/or functional diseases and disorders (e.g., neurological syndrome).

Examples of neurological disorders include, but are not limited to, headache, stupor and coma, dementia, seizure, sleep disorders, trauma, infections, neoplasms, neuroopthalmology, movement disorders, demyelinating diseases, spinal cord disorders, and disorders of peripheral nerves, muscle and neuromuscular junctions. Addiction and mental illness, include, but are not limited to, bipolar disorder and schizophrenia, are also included in the definition of neurological disorder. The following is a list of several neurological disorders, symptoms, signs and syndromes that can be treated using compositions and methods according to the present invention: acquired epileptiform aphasia; acute disseminated encephalomyelitis; adrenoleukodystrophy; agenesis of the corpus callosum; agnosia: Aicardi syndrome; Alexander disease; Alpers' disease; alternating hemiplegia; vascular dementia; amyotrophic lateral sclerosis; anencephaly; Angelman syndrome; angiomatosis; anoxia; aphasia; apraxia; arachnoid cysts; arachnoiditis; Anronl-Chiari malformation; arteriovenous malformation; Asperger syndrome; ataxia telegiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm: Bloch Sulzberger syndrome; brachial plexus injury; brain abscess; brain injury; Brown-Sequard syndrome; Cana van disease; carpal tunnel syndrome; causalgia; central pain syndrome; central pontine myelinolysis: cephalic disorder; cerebral aneurysm; cerebral arteriosclerosis: cerebral atrophy; cerebral gigantism; cerebral palsy; Charcot-Marie-Tooth disease; Chiari malformation; chorea; chronic inflammatory demyelinating polyneuropathy; chronic pain; chronic regional pain syndrome; Coffin Lowry syndrome; coma, including persistent vegetative state; congenital facial diplegia; corticobasal degeneration: cranial arteritis; craniosynostosis; Creutzfeldt-Jakob disease; cumulative trauma disorders; Cushing's syndrome; cytomegalic inclusion body disease; cytomegalovirus infection; dancing eyes-dancing feet syndrome; Dandy Walker syndrome; Dawson disease; De Moisier's syndrome; Dejerine-Klumke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; fronto-temporal dementia; Gaucher's disease; Gerstmann's syndrome; giant cell arteritis; giant cell inclusion disease; globoid cell leukodystrophy; Guillain-Barre syndrome; HTLV-1-associated myelopathy; Hallervorden-Spatz disease; head injury; headache; hemifacial spasm; hereditary spastic paraplegia: heredopathia atactic a polyneuritiformis; herpes zoster oticus; herpes zoster; Hirayama syndrome; HIV associated dementia and neuropathy (also neurological manifestations of AIDS); holoprosencephaly; Huntington's disease and other polyglutamine repeat diseases; hydranencephaly: hydrocephalus; hypercortisolism; hypoxia; immune-mediated encephalomyelitis; inclusion body myositis; incontinentia pigmenti; infantile phytanic acid storage disease; infantile spasms; inflammatory myopathy; intracranial cyst; intracranial hypertension; Joubert syndrome; Kearns-Sayre syndrome; Kennedy disease Kinsboume syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gustaut syndrome; Lesch-Nyhan syndrome; leukodystrophy; Lewy body dementia; Lissencephaly; locked-in syndrome; Lou Gehrig's disease (i.e., motor neuron disease or amyotrophic lateral sclerosis); lumbar disc disease; Lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neuron disease; Moyamoya disease; mucopolysaccharidoses; milti-infarct dementia; multifocal motor neuropathy; multiple sclerosis and other demyelinating disorders; multiple system atrophy with postural hypotension; p muscular dystrophy; myasthenia gravis; myelmociastic diffuse sclerosis; myoclonic encephalopathy of infants; myoclonus; myopathy; myotonia congenital; narcolepsy: neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Neurodegenerative disease or disorder (Parkinson's disease, Huntington's disease, Alzheimer's disease, amyotrophic lateral sclerosis (ALS), dementia, multiple sclerosis and other diseases and disorders associated with neuronal cell death); paramyotonia congenital; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; porencephaly; post-polio syndrome; postherpetic neuralgia; postinfectious encephalomyelitis; postural hypotension; Prader-Willi syndrome; primary lateral sclerosis; prion diseases; progressive hemifacial atrophy; progressive multifocalleukoencephalopathy; progressive sclerosing poliodystrophy; progressive supranuclear palsy; Ramsay-Hunt syndrome (types I and II); Rasmussen's encephalitis: reflex sympathetic dystrophy syndrome; Refsum disease; repetitive motion disorders, repetitive stress injuries; restless legs syndrome; retrovirus-associated myelopathy; Rett syndrome; Reye's syndrome; Saint Vitus dance; Sandhoff disease; Schilder's disease; schizencephaly; septo-optic dysplasia: shaken baby syndrome; shingles: Shy-Drager syndrome; Sjogren's syndrome; Soto's syndrome; spasticity; spina bifida; spinal cord injury; spinal muscular atrophy; Stiff-Person syndrome; stroke; Sturge-Weber syndrome; subacute sclerosing panencephalitis; subcortical arteriosclerotic encephalopathy; Sydenham chorea; syncope; syringomyelia; tardive dyskinesia; Tay-Sachs disease; temporal arteritis; tethered spinal cord syndrome; Thomsen disease; thoracic outlet syndrome; Tic Douloureux; Todd's paralysis; Tourette syndrome; transient ischemic attack; transmissible spongiform encephalopathies; transverse myelitis; traumatic brain injury; tremor, trigeminal neuralgia; tropical spastic paraparesis: tuberous sclerosis; vascular dementia (multi-infarct dementia); vasculitis including temporal arteritis; Von Hippel-Liodau disease; Wallenberg's syndrome; Werdnig-Hoffman disease; West syndrome; Williams syndrome; Wildoris disease; and Zellweger syndrome.

In some embodiments, the neurological disease or neurological disorder is Alzheimer's disease, Down syndrome, Alzheimer's disease associated with Down syndrome, Parkinson's disease, ALS, dementia, Huntington's disease, multiple sclerosis, proximal lateral sclerosis, stroke, stroke, or mild cognitive impairment.

In some embodiments, the dementia may be Alzheimer's dementia, cerebrovascular dementia, dementia due to head injury, multi-infarct dementia, mixed or alcoholic dementia of Alzheimer's disease and multi-infarct dementia.

The ability of test compounds to act as inhibitors of DYRK1A may be demonstrated by assays known in the art. The activity of the compounds and compositions provided herein as DYRK1A inhibitors can be assayed in vitro, in vivo, or in a cell line. In vitro assays include assays that determine inhibition of the kinase. Alternate in vitro assays quantitate the ability of the inhibitor to bind to the protein kinase and can be measured either by radio labelling the compound prior to binding, isolating the compound/kinase complex and determining the amount of radio label bound, or by running a competition experiment where new compounds are incubated with the kinase bound to known radio ligands.

Potency of a DYRK1A inhibitor as provided herein can be determined by EC₅₀ or IC₅₀ values. A compound with a lower EC₅₀ or IC₅₀ value, as determined under substantially similar conditions, is a more potent inhibitor relative to a compound with a higher EC₅₀ or IC₅₀ value.

In some embodiments, the substantially similar conditions comprise determining a DYRK1A-dependent phosphorylation level, in vitro or in vivo (e.g., in neural cells, such as neurons, astrocytes, oligodendrocytes, microglia, ependymal cells, Schwann cells, and satellite cells, expressing a wild type DYRK1A, a mutant DYRK1A, or a fragment of any thereof).

Potency of a DYRK1A inhibitor as provided herein can also be determined by IC₅₀ value. A compound with a lower IC₅₀ value, as determined under substantially similar conditions, is a more potent inhibitor relative to a compound with a higher IC₅₀ value. In some embodiments, the substantially similar conditions comprise determining a DYRK1A-dependent phosphorylation level, in vitro or in vivo (e.g., in neural cells, such as neurons, astrocytes, oligodendrocytes, microglia, ependymal cells, Schwann cells, and satellite cells, expressing a wild type DYRK1A, a mutant DYRK1A, or a fragment of any thereof).

As used herein, terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, the terms “subject,” “individual,” or “patient,” are used interchangeably, refers to any animal, including mammals such as mice, rats, other rodents, rabbits, dogs, cats, swine, cattle, sheep, horses, primates, and humans. In some embodiments, the subject is a human. In some embodiments, the subject has experienced and/or exhibited at least one symptom of the disease or disorder to be treated and/or prevented.

In some embodiments, the subject has been identified or diagnosed as having a neurological disorder with a dysregulation of a DYRK1A gene, a DYRK1A protein, or expression or activity, or level of any of the same (a DYRK1A-associated neurological disorder) (e.g., as determined using a regulatory agency-approved, e.g., FDA-approved, assay or kit). The subject can be a subject that is positive for a dysregulation of a DYRK1A gene, a DYRK1A protein, or expression or activity, or level of any of the same (e.g., identified as positive using a regulatory agency-approved, e.g., FDA-approved, assay or kit). In some embodiments, the subject is suspected of having a DYRK1A-associated neurological disorder.

In some embodiments, the subject has a clinical record indicating that the subject has a neurological disorder that has a dysregulation of a DYRK1A gene, a DYRK1A protein, or expression or activity, or level of any of the same (and optionally the clinical record indicates that the subject should be treated with any of the compositions provided herein).

In certain embodiments, compounds of Formula (I), (II), (III), (IV), or pharmaceutically acceptable salts of any of the foregoing, are useful for preventing neurological disorders as defined herein (for example, Alzheimer's disease). The term “preventing” as used herein means to delay the onset, recurrence or spread, in whole or in part, of the disease or condition as described herein, or a symptom thereof.

The term “DYRK1A-associated neurological disorder” as used herein refers disorders associated with or having a dysregulation of a DYRK1A gene, a DYRK1A protein, or the expression or activity or level of any (e.g., one or more) of the same (e.g., any of the types of dysregulation of a DYRK1A gene, or a DYRK1A protein, or the expression or activity or level of any of the same described herein). Non-limiting examples of a DYRK1A-associated disease or disorder include, for example, Down Syndrome, Alzheimer's disease, and Alzheimer's disease associated with Down Syndrome.

The phrase “dysregulation of a DYRK1A gene, a DYRK1A protein, or the expression or activity or level of any of the same” refers to a gene duplication (or multiplication) that results in an increased level of DYRK1A in a cell, or a mutation in a regulatory sequence (e.g., a promoter and/or enhancer) that results in an increased level of DYRK1A in a cell), or increased expression (e.g., increased levels) of a wild type DYRK1A in a mammalian cell due to aberrant cell signaling and/or dysregulated autocrine/paracrine signaling (e.g., as compared to a control cell lacking the aberrant signaling).

Some embodiments provide a method for treating a neurological disorder in a subject in need thereof, the method comprises administering to the subject a therapeutically effective amount of a compound of Formula (I), (II), (III), (IV), or a pharmaceutically acceptable salt of any of the foregoing, or a pharmaceutical composition comprising Formula (I), (II), (III), or (IV), or a pharmaceutically acceptable salt of any of the foregoing.

In some embodiments, the method for treating a neurological disorder in a subject in need thereof, comprises (a) determining that the neurological disorder is associated with a dysregulation of a DYRK1A gene, a DYRK1A protein, or expression or activity or level of any of the same; and (b) administering to the subject a therapeutically effective amount of a compound of Formula (I), (II), (III), (IV), or a pharmaceutically acceptable salt of any of the foregoing, or a pharmaceutical composition comprising Formula (I), (II), (III), or (IV), or a pharmaceutically acceptable salt of any of the foregoing.

Some embodiments provide a method of treating a DYRK1A-associated neurological disorder in a subject, the method comprises administering to a subject identified or diagnosed as having a DYRK1A-associated neurological disorder a therapeutically effective amount of a compound of Formula (I), (II), (III), (IV), or a pharmaceutically acceptable salt of any of the foregoing, or a pharmaceutical composition comprising Formula (I), (II), (III), or (IV), or a pharmaceutically acceptable salt of any of the foregoing.

In some embodiments, the method of treating a DYRK1A-associated neurological disorder in a subject, comprises:
(a) determining that the neurological disorder in the subject is a DYRK1A-associated neurological disorder; and
(b) administering to the subject a therapeutically effective amount of a compound of Formula (I), (II), (III), (IV), or a pharmaceutically acceptable salt of any of the foregoing, or a pharmaceutical composition comprising Formula (I), (II), (III), or (IV), or a pharmaceutically acceptable salt of any of the foregoing.

Some embodiments provide a method of treating a subject, the method comprises administering a therapeutically effective amount of a compound of Formula (I), (II), (III), (IV), or a pharmaceutically acceptable salt of any of the foregoing, or a pharmaceutical composition comprising Formula (I), (II), (III), (IV), or a pharmaceutically acceptable salt of any of the foregoing, to a subject having a clinical record that indicates that the subject has a dysregulation of a DYRK1A gene, DYRK1A protein, or expression or activity or level of any of the same.

In some embodiments, the method comprises the step of determining that the neurological disorder in the subject is a DYRK1A-associated neurological disorder and includes performing an assay to detect dysregulation in a DYRK1A gene, a DYRK1A protein, or expression or activity or level of any of the same in a sample from the subject.

Some embodiments provide a method for treating a neurological disorder in a subject in need thereof, the method comprising (a) determining that the neurological disorder is associated with Down Syndrome; and (b) administering to the subject a therapeutically effective amount of a compound of Formula (I), (II), (III), (IV), or a pharmaceutically acceptable salt of any of the foregoing, or a pharmaceutical composition comprising Formula (I), (II), (III), or (IV), or a pharmaceutically acceptable salt of any of the foregoing.

In some embodiments, the step of determining that the neurological disorder in the subject is associated with Down Syndrome includes performing an assay on a sample from the subject.

In some embodiments, the method further comprises obtaining a sample from the subject. In some embodiments, the sample is a blood sample. In some embodiments, the sample is a sample of cerebrospinal fluid (CSF).

In some embodiments, the assay is selected from the group consisting of sequencing, immunohistochemistry, enzyme-linked immunosorbent assay, and fluorescence in situ hybridization (FISH).

In some embodiments, the FISH is break apart FISH analysis. In some embodiments, the sequencing is pyrosequencing or next generation sequencing.

In some embodiments, the DYRK1A-associated neurological disorder is selected from the group consisting of Down Syndrome, Alzheimer's disease, and Alzheimer's disease associated with Down syndrome. In some embodiments, the DYRK1A-associated neurological disorder is Alzheimer's disease associated with Down syndrome.

In some embodiments, the method further comprises administering to the subject an additional therapy or therapeutic agent as described herein.

Some embodiments provide a method for modulating DYRK1A in a mammalian cell, the method comprises contacting the mammalian cell with a therapeutically effective amount of a compound of a Formula (I), (II), (III), or (IV). or a pharmaceutically acceptable salt of any of the foregoing.

In some embodiments, the contacting occurs in vivo. In some embodiments, the contacting occurs in vitro. In some embodiments, the mammalian cell is a mammalian neural cell. In some embodiments, the mammalian neural cell is a mammalian DYRK1A-associated neural cell. In some embodiments, the cell has a dysregulation of a DYRK1A gene, a DYRK1A protein, or expression or activity or level of any of the same. In some embodiments, the cell has a chromosomal abnormality associated with Down Syndrome.

Exemplary Sequence of Human Dual specificity tyrosine-phosphorylation-regulated kinase TA (UniProtKB entry Q13627) (SEQ ID NO: 1)

MHTGGETSAC KPSSVRLAPS FSFHAAGLQM AGQMPHSHQY
SDRRQPNISD QQVSALSYSD QIQQPLTNQV MPDIVMLQRR
MPQTFRDPAT APLRKLSVDL IKTYKHINEV YYAKKKRRHQ
QGQGDDSSHK KERKVYNDGY DDDNYDYIVK NGEKWMDRYE
IDSLIGKGSF GQVVKAYDRV EQEWVAIKII KNKKAFLNQA
QIEVRLLELM NKHDTEMKYY IVHLKRHFMF RNHLCLVFEM
LSYNLYDLLR NTNFRGVSLN LTRKFAQQMC TALLFLATPE
LSIIHCDLKP ENILLCNPKR SAIKIVDFGS SCQLGQRIYQ
YIQSRFYRSP EVLLGMPYDL AIDMWSLGCI LVEMHTGEPL
FSGANEVDQM NKIVEVLGIP PAHILDQAPK ARKFFEKLPD
GTWNLKKTKD GKREYKPPGT RKLHNILGVE TGGPGGRRAG
ESGHTVADYL KFKDLILRML DYDPKTRIQP YYALQHSFFK
KTADEGTNTS NSVSTSPAME QSQSSGTTSS TSSSSGGSSG
TSNSGRARSD PTHQHRHSGG HFTAAVQAMD CETHSPQVRQ
QFPAPLGWSG TEAPTQVTVE THPVQETTFH VAPQQNALHH
HHGNSSHHHH HHHHHHHHHG QQALGNRTRP RVYNSPTNSS
STQDSMEVGH SHHSMTSLSS STTSSSTSSS STGNQGNQAY
QNRPVAANTL DFGQNGAMDV NLTVYSNPRQ ETGIAGHPTY
QFSANTGPAH YMTEGHLTMR QGADREESPM TGVCVQQSPV
ASS

In some embodiments, compounds of Formula (I), (II), (III), or (IV). or pharmaceutically acceptable salt of any of the foregoing, are useful for treating a neurological disorder that has been identified as being associated with dysregulation of DYRK1A.

Accordingly, provided herein are methods for treating a subject diagnosed with (or identified as having) a neurological disorder that include administering to the subject a therapeutically effective amount of a compound of Formula (I), (II), (III), or (IV), or a pharmaceutically acceptable salt of any of the foregoing.

Also provided herein are methods for treating a subject identified or diagnosed as having a DYRK1A-associated neurological disorder that include administering to the subject a therapeutically effective amount of a compound of Formula (I), (II), (III), or (IV), or a pharmaceutically acceptable salt of any of the foregoing, or a pharmaceutical composition thereof. In some embodiments, the subject that has been identified or diagnosed as having a DYRK1A-associated neurological disorder through the use of a regulatory agency-approved, e.g., FDA-approved test or assay for identifying dysregulation of a DYRK1A gene, a DYRK1A protein, or expression or activity or level of any of the same, in a subject or a biological sample (e.g., blood and/or CSF) from the subject or by performing any of the non-limiting examples of assays described herein. In some embodiments, the test or assay is provided as a kit. In some embodiments, the neurological disorder is a DYRK1A-associated neurological disorder.

The term “regulatory agency” refers to a country's agency for the approval of the medical use of pharmaceutical agents with the country. For example, a non-limiting example of a regulatory agency is the U.S. Food and Drug Administration (FDA).

Also provided is a method for inhibiting DYRK1A activity in a cell, comprising contacting the cell with a compound of Formula (I), (II), (III), or (IV), or a pharmaceutically acceptable salt of any of the foregoing. In some embodiments, the contacting is in vitro. In some embodiments, the contacting is in vivo. In some embodiments, the contacting is in vivo, wherein the method comprises administering a therapeutically effective amount of a compound of Formula (I), (II), (III), or (IV), or a pharmaceutically acceptable salt of any of the foregoing, to a subject having a cell having aberrant DYRK1A activity. In some embodiments, the cell is a neural cell. In some embodiments, the neural cell is a DYRK1A-associated neural cell.

As used herein, the term “contacting” refers to the bringing together of indicated moieties in an in vitro system or an in vivo system. For example, “contacting” a DYRK1A protein with a compound provided herein includes the administration of a compound provided herein to an individual or subject, such as a human, having a DYRK1A protein, as well as, for example, introducing a compound provided herein into a sample containing a cellular or purified preparation containing the DYRK1A protein.

The phrase “therapeutically effective amount” means an amount of compound that, when administered to a subject in need of such treatment, is sufficient to (i) treat a DYRK1A protein-associated disease or disorder, (ii) attenuate, ameliorate, or eliminate one or more symptoms of the particular disease, condition, or disorder, or (iii) delay the onset of one or more symptoms of the particular disease, condition, or disorder described herein. The amount of a compound of Formula (I), (II), (III), or (IV), or a pharmaceutically acceptable salt of any of the foregoing, that will correspond to such an amount will vary depending upon factors such as the particular compound, disease condition and its severity, the identity (e.g., weight) of the subject in need of treatment, but can nevertheless be routinely determined by one skilled in the art.

When employed as pharmaceuticals, the compounds of Formula (I), (II), (III), or (IV), including pharmaceutically acceptable salts o any of the foregoing, can be administered in the form of pharmaceutical compositions as described herein.

### Combinations

In some embodiments, of any of the methods described herein, the compound of Formula (I), (II), (III), or (IV), or a pharmaceutically acceptable salt of any of the foregoing, is administered in combination with a therapeutically effective amount of at least one additional therapeutic agent selected from one or more additional therapies or therapeutic agents.

In some embodiments, the methods described herein further comprise administering one or more additional therapies selected from typical antipsychotics, atypical antipsychotics, antidepressants, electroconvulsive therapy, transcranial magnetic stimulation, benzodiazepines, mood stabilizers, cholinesterase inhibitors, memantine, NSAIDs, analgesics, anxiolytics, gabapentin and pregabalin.

In some embodiments, the methods described herein further comprise providing cognitive behavior therapy to the subject.

In some embodiments, the one or more additional therapies is a standard of care treatment for neuropathic pain. In some embodiments, the one or more additional therapies is a standard of care treatment for Alzheimer's disease. In some embodiments, the one or more additional therapies is a standard of care treatment for Alzheimer's disease associated with Down Syndrome.

In some embodiments, the one or more additional therapies is a typical antipsychotic. Representative typical antipsychotics include, but are not limited to chlorpromazine, chlorprothixene, levomepromazine, mesoridazine, periciazine, promazine, loxapine, molindone, perphenazine, thiothixene, droperidol, flupentixol, fluphenazine, haloperidol, pimozide, prochlorperazine, thioproperazine, trifluoperazine, and zuclopenthixol.

In some embodiments, the one or more additional therapies is an atypical antipsychotic. Representative atypical antipsychotics include, but are not limited to aripiprazole, risperidone, olanzapine, quetiapine, asenapine, paliperidone, ziprasidone, or lurasidone.

In some embodiments, the one or more additional therapies is an antidepressant. In some embodiments, the antidepressant is an atypical antidepressant, a selective serotonin reuptake inhibitor, a selective serotonin and norepinephrine reuptake inhibitor, a monoamine oxidase inhibitor, a selective norepinephrine reuptake inhibitor, or a tricyclic antidepressant.

In some embodiments, the compound of Formula (I), (II), (III), or (IV), or a pharmaceutically acceptable salt of any of the foregoing, and the one or more additional therapies are administered as separate dosages sequentially in any order. In some embodiments, the compound of Formula (I), (II), (III), or (IV), or a pharmaceutically acceptable salt of any of the foregoing, and the one or more additional therapies are administered as a single dosage form.

In some embodiments, the antidepressant is an atypical antidepressant. Representative atypical antidepressants include, but are not limited to mirtazapine, mianserin, bupropion, trazodone, nefazodone, tianeptine, opipramol, agomelatine, vilazodone, and vortioxetine.

In some embodiments, the antidepressant is a selective serotonin reuptake inhibitor. Representative selective serotonin reuptake inhibitors include, but are not limited to citalopram, escitalopram, fluoxetine, fluvoxamine, paroxetine, and sertraline.

In some embodiments, the antidepressant is a selective serotonin and norepinephrine reuptake inhibitor. Representative selective serotonin and norepinephrine reuptake inhibitors include, but are not limited to atomoxetine, desvenlafaxine, duloxetine, levomilnacipran, milnacipran, sibutramine, tramadol, and venlafaxine.

In some embodiments, the antidepressant is a monoamine oxidase inhibitor. Representative monoamine oxidase inhibitors include, but are not limited to moclobemide, rasagiline, selegiline, or safinamide.

In some embodiments, the antidepressant is a selective norepinephrine reuptake inhibitor. Representative selective norepinephrine reuptake inhibitors include, but are not limited to reboxetine.

In some embodiments, the antidepressant is a tricyclic antidepressant. Representative tricyclic antidepressants include, but are not limited to amineptine, amitriptyline, amoxapine, butriptyline, clomipramine, desipramine, dibenzepin, dosulepin, doxepin, imipramine, iprindole, lofepramine, maprotiline, norclomipramine, northiaden, nortriptyline, pipramol, protriptyline, tianeptine, and trimipramine.

In some embodiments, the one or more additional therapies is a benzodiazepine. Representative benzodiazepines include, but are not limited to alprazolam, bromazepam, chlordiazepoxide, clonazepam, clorazepate, diazepam, flurazepam, lorazepam, oxazepam, temazepam, or triazolam.

In some embodiments, the one or more additional therapies is a mood stabilizer. Representative mood stabilizers include, but are not limited to lithium, valproic acid, lamotrigine, or carbamazepine. In some embodiments, the one or more additional therapies is electroconvulsive therapy or transcranial magnetic stimulation.

In some embodiments, the one or more additional therapies is sertraline. In some embodiments, the one or more additional therapies is venlafaxine.

In some embodiments, the one or more additional therapies is a cholinesterase inhibitor. Representative cholinesterase inhibitors include, but are not limited to donepezil, galantamine, and rivastigmine.

In some embodiments, the one or more additional therapies is memantine.

In some embodiments, the one or more additional therapies is an NSAID. Representative NSAIDs include, but are not limited to clonixin, licofelone, salicylates (such as aspirin and diflunisal), propionic acid derivative (such as ibuprofen, dexibuprofen, naproxen, fenoprofen, ketoprofen, dexketoprofen, flurbiprofen, and oxaprozin), acetic acid derivatives (such as indomethacin, tolmetin, sulindac, etodolac, ketorolac, diclofenac, aceclofenac, and bromfenac), and COX-2 inhibitors (such as celecoxib).

In some embodiments, the one or more additional therapies is an analgesic. Representative analgesics include, but are not limited to nefopam, flupiritine, ziconotide, acetaminophen, and opioids (such as morphine, oxycodone, methadone, codeine, fentanyl, hydrocodone, and tramadol).

In some embodiments, the one or more additional therapies is an anxiolytic. Representative anxiolytics include, but are not limited to alnespirone, adinazolam, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazepam, quazepam, reclazepam, tracazolate, trepipam, temazepam, triazolam, uldazepam, and zolazepam.

In some embodiments, the one or more additional therapies is gabapentin or pregabalin.

In some embodiments, the one or more additional therapies is one additional therapy. In some embodiments, the one or more additional therapies is two, three, or four additional therapies.

Some embodiments provide a method of treating a neurological disorder, comprising administering a compound of Formula (I), (II), (III), or (IV), or a pharmaceutically acceptable salt of any of the foregoing, and one or more additional therapies selected from typical antipsychotics, atypical antipsychotics, antidepressants, electroconvulsive therapy, transcranial magnetic stimulation, benzodiazepines, mood stabilizers, cholinesterase inhibitors, memantine, NSAIDs, analgesics, anxiolytics, gabapentin and pregabalin, to a subject in need thereof.

In some embodiments, the subject was being administered the one or more additional therapies prior to initiation of treatment with a compound of Formula (I), (II), (III), or (IV), or a pharmaceutically acceptable salt of any of the foregoing. In some embodiments, the subject was being administered the one or more additional therapies prior to initiation of treatment with a compound of Formula (I), (II), (III), or (IV), or a pharmaceutically acceptable salt of any of the foregoing, but after treatment with a compound of Formula (I), (II), (III), or (IV), or a pharmaceutically acceptable salt of any of the foregoing, for a period of time, the subject is no longer administered the one or more additional therapies. In some embodiments, of this paragraph, the period of time is about 1 month to about 1 year, for example, about 1 month to about 5 months, about 3 months to about 8 months, about 7 months to about 1 year, about 1 month, about 2 months, about 3 months, about 4 months, about 5 months, about 6 months, about 7 months, about 8 months, about 9 months, about 10 months, about 11 months, about 12 months, or any value in between. In some embodiments, the amount of the one or more additional therapies is decreased during the period of time, to zero at the end of the period of time.

In some embodiments, the subject has previously been administered one or more additional therapies selected from typical antipsychotics, atypical antipsychotics, antidepressants, electroconvulsive therapy, transcranial magnetic stimulation, benzodiazepines, mood stabilizers, cholinesterase inhibitors, memantine, NSAIDs, analgesics, anxiolytics, gabapentin, and pregabalin; wherein the subject was not responsive to the previous one or more therapies.

In some embodiments, the subject has previously been administered a standard of care treatment for neuropathic pain and the subject was not responsive to the previous therapy. In some embodiments, the subject has previously been administered a standard of care treatment for Alzheimer's disease and the subject was not responsive to the previous therapy. In some embodiments, the subject has previously been administered a standard of care treatment for Alzheimer's disease associated with Down Syndrome and the subject was not responsive to the previous therapy.

In some embodiments, the subject has previously been administered one or more additional therapies selected from typical antipsychotics, atypical antipsychotics, antidepressants, electroconvulsive therapy, transcranial magnetic stimulation, benzodiazepines, mood stabilizers, cholinesterase inhibitors, memantine, NSAIDs, analgesics, anxiolytics, gabapentin, and pregabalin, and was not responsive to the previous therapy.

In some embodiments, the subject has previously been administered one or more typical antipsychotics such as chlorpromazine, chlorprothixene, levomepromazine, mesoridazine, periciazine, promazine, loxapine, molindone, perphenazine, thiothixene, droperidol, flupentixol, fluphenazine, haloperidol, pimozide, prochlorperazine, thioproperazine, trifluoperazine, and zuclopenthixol, and was not responsive to the previous therapy.

In some embodiments, the subject has previously been administered one or more atypical antipsychotics, such as aripiprazole, risperidone, olanzapine, quetiapine, asenapine, paliperidone, ziprasidone, or lurasidone, and was not responsive to the previous therapy.

In some embodiments, the subject has previously been administered one or more antidepressants and was not responsive to the previous therapy. In some embodiments, the antidepressant is an atypical antidepressant, a selective serotonin reuptake inhibitor, a selective serotonin and norepinephrine reuptake inhibitor, a monoamine oxidase inhibitor, a selective norepinephrine reuptake inhibitor, or a tricyclic antidepressant, and was not responsive to the previous therapy.

In some embodiments, the subject has previously been administered one or more atypical antidepressants, such as mirtazapine, mianserin, bupropion, trazodone, nefazodone, tianeptine, opipramol, agomelatine, vilazodone, and vortioxetine, and was not responsive to the previous therapy.

In some embodiments, the subject has previously been administered one or more selective serotonin reuptake inhibitors, such as citalopram, escitalopram, fluoxetine, fluvoxamine, paroxetine, and sertraline, and was not responsive to the previous therapy.

In some embodiments, the subject has previously been administered one or more selective serotonin and norepinephrine reuptake inhibitors, such as atomoxetine, desvenlafaxine, duloxetine, levomilnacipran, milnacipran, sibutramine, tramadol, and venlafaxine, and was not responsive to the previous therapy.

In some embodiments, the subject has previously been administered one or more monoamine oxidase inhibitors, such as moclobemide, rasagiline, selegiline, or safinamide, and was not responsive to the previous therapy.

In some embodiments, the subject has previously been administered one or more selective norepinephrine reuptake inhibitors, such as reboxetine, and was not responsive to the previous therapy.

In some embodiments, the subject has previously been administered one or more tricyclic antidepressants, such as amineptine, amitriptyline, amoxapine, butriptyline, clomipramine, desipramine, dibenzepin, dosulepin, doxepin, imipramine, iprindole, lofepramine, maprotiline, norclomipramine, northiaden, nortriptyline, pipramol, protriptyline, tianeptine, and trimipramine, and was not responsive to the previous therapy.

In some embodiments, the subject has previously been administered one or more benzodiazepines, such as alprazolam, bromazepam, chlordiazepoxide, clonazepam, clorazepate, diazepam, flurazepam, lorazepam, oxazepam, temazepam, or triazolam, and was not responsive to the previous therapy.

In some embodiments, the subject has previously been administered one or more mood stabilizers, such as lithium, valproic acid, lamotrigine, or carbamazepine, and was not responsive to the previous therapy.

In some embodiments, the one or more additional therapies is electroconvulsive therapy or transcranial magnetic stimulation, and was not responsive to the previous therapy.

In some embodiments, the subject has previously been administered sertraline, and was not responsive to the previous therapy. In some embodiments, the subject has previously been administered venlafaxine, and was not responsive to the previous therapy.

In some embodiments, the subject has previously been administered one or more cholinesterase inhibitors such as donepezil, galantamine, or rivastigmine, and was not responsive to the previous therapy.

In some embodiments, the subject has previously been administered memantine, and was not responsive to the previous therapy.

In some embodiments, the subject has previously been administered one or more NSAIDs such as clonixin, licofelone, aspirin, diflunisal, ibuprofen, dexibuprofen, naproxen, fenoprofen, ketoprofen, dexketoprofen, flurbiprofen, oxaprozin, indomethacin, tolmetin, sulindac, etodolac, ketorolac, diclofenac, aceclofenac, bromfenac), or celecoxib, and was not responsive to the previous therapy.

In some embodiments, the subject has previously been administered one or more analgesics such as nefopam, flupiritine, ziconotide, acetaminophen, morphine, oxycodone, methadone, codeine, fentanyl, hydrocodone, or tramadol, and was not responsive to the previous therapy.

In some embodiments, the subject has previously been administered one or more anxiolytics, such as alnespirone, adinazolam, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazepam, quazepam, reclazepam, tracazolate, trepipam, temazepam, triazolam, uldazepam, or zolazepam, and was not responsive to the previous therapy.

In some embodiments, the subject has previously been administered gabapentin or pregabalin, and was not responsive to the previous therapy.

In some embodiments, the one or more additional therapies previously administered to the subject is 1-3 additional therapies. In some embodiments, the one or more additional therapies previously administered to the subject is one additional therapy. In some embodiments, the one or more additional therapies previously administered to the subject is two additional therapies. In some embodiments, the one or more additional therapies previously administered to the subject is three additional therapies.

Subjects that were “non-responsive” to a previous therapy includes subjects where the previous therapy lacked sufficient clinical efficacy, subjects that experienced an unacceptable number and/or severity of side effects due to the previous therapy (sufficient to require discontinuation of treatment), and subjects that experienced both of the foregoing. Side effects include, but are not limited to weight gain, flattened affect, tardive dyskinesia, drowsiness, nausea, vomiting, constipation, dry mouth, restlessness, dizziness, loss of sexual desire, erectile dysfunction, insomnia, and blurred vision.

### Embodiments

### Exemplary Embodiments of Compounds of Formula (I)

Embodiment 1: A compound of Formula (I):

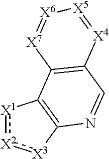

or a pharmaceutically acceptable salt thereof, wherein:
each dashed line represents a single bond or a double bond;
X¹ is CR^{1A}R^{1B}, NR^{A}, S, or O;
X² is CR^{2A}R^{2B}, C(═O), or NR^{B};
X³ is CR^{3A}R^{3B}, NR^{C}, S, or O;
X⁴ is CR⁴ or N;
X⁵ is CR⁵ or N;
X⁶ is CR⁶ or N;
X⁷ is CH, CF, or N;
R^{1A} is hydrogen, C1-C6 alkyl, —C(═O)NR^{F}R^{G}, —(C0-C6 alkyl)-5-6 membered heteroaryl,
   —C(═O)-3-6 membered heterocyclyl optionally substituted with C1-C6 alkyl, a phenyl optionally substituted with halogen or —CO₂H, a 3-6 membered heterocyclyl optionally substituted with C1-C6 alkyl or —S(O₂)—C1-C6 alkyl, or a C3-C6 cycloalkyl optionally substituted with hydroxyl,
   —C(═O)NR^{F}R^{G}, —NR^{F}R^{G}, —CO₂R^{H}, or C1-C6 alkoxy;
R^{1B} is hydrogen or absent, wherein R^{1B} is absent when x¹ [symbol] ² is a double bond;
R^{2A} is hydrogen, C1-C6 alkyl, C1-C6 haloalkyl, C3-C6 cycloalkyl, 5-6 membered heteroaryl, a phenyl optionally substituted with —CO₂H, or a 3-6 membered heterocyclyl optionally substituted with C1-C6 alkyl, C1-C6 haloalkyl, C1-C6 hydroxyalkyl, or —(C0-C6 alkyl)-3-6 membered cycloalkyl optionally substituted with —CO₂H;
R^{2B} is hydrogen or absent, wherein R^{2B} is absent when either of x¹ [symbol] x² or x² [symbol] x³ is a double bond;
R^{3A} is hydrogen;
R^{3B} is hydrogen, C1-C6 alkyl, or absent, wherein R^{3B} is absent when x² [symbol] x³ is a double bond;
R⁴ is hydrogen, halogen, C1-C6 alkyl, or 3-6 membered heterocyclyl optionally substituted with C1-C6 alkyl;
R⁵ is hydrogen, —CO₂H, —C(═O)OCH₃, C1-C6 alkyl optionally substituted with hydroxyl, or a 5-6 membered heteroaryl optionally substituted with C1-C6 alkyl;
R⁶ is hydrogen or a 5-6 membered heteroaryl optionally substituted with 1-2 substituents independently selected from R^{D}, C1-C6 alkyl, and 4-6 membered heterocyclyl optionally substituted with hydroxyl;
R^{A} and R^{B} are independently absent, hydrogen, C1-C6 alkyl optionally substituted with 3-6 membered heterocyclyl or 5-6 membered heteroaryl, or a C3-C6 cycloalkyl optionally substituted with hydroxyl or C1-C6 alkoxy;
R^{C} is absent, hydrogen, or methyl;
R^{D} is

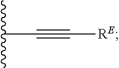

R^{E} is a C1-C6 alkyl, phenyl, 5-6 membered heteroaryl, or 4-6 membered heterocyclyl; wherein the C1-C6 alkyl, phenyl, 5-6 membered heteroaryl, and 4-6 membered heterocyclyl are each optionally substituted with 1-2 independently selected R^{I};
each R^{F} and R^{G} are independently selected from hydrogen and C1-C6 alkyl; or R^{F} and R^{G} together with the nitrogen atom to which they are attached form a 4-6 membered heterocyclyl optionally substituted with C1-C6 alkyl;
R^{H} is hydrogen or C1-C6 alkyl;
each R^{I} is independently C1-C6 alkyl, C1-C6 alkoxy, halogen, hydroxyl, cyano, or trifluoromethyl.

Embodiment 2: The compound of Embodiment 1, wherein X¹ is CR^{1A}R^{1B}, X² is CR^{2A}R^{2B}, X³ is NR^{C}, x¹ [symbol] x² is a double bond, x² [symbol] x³ is a single bond, RIB is absent, and R²B is absent.

Embodiment 3: The compound of Embodiment 1, wherein X¹ is NR^{A}, X² is CR^{2A}R^{2B}, X³ is NR^{C}, x¹ [symbol] x² is a single bond, x² [symbol] x³ is a double bond, and R^{2B} and R^{C} are absent.

Embodiment 4: The compound of Embodiment 1, wherein X¹ is NR^{A}, X² is CR^{2A}R^{2B}X³ is NR^{C}, x¹ [symbol] x² is a double bond, X² [symbol] x³ is a single bond, and R^{2B} and R^{A} is absent.

Embodiment 5: The compound of Embodiment 1, wherein X¹ is S, X² is CR^{2A}R^{2B}, X³ is CR^{3A}R^{3B} x¹ [symbol] x² is a single bond, x² [symbol] x³ is a double bond, R^{2B} is absent, and R^{3B} is absent.

Embodiment 6: The compound of Embodiment 1, wherein X¹ is S, X² is CR^{2A}R^{2B}, X³ is NR^{C}, x¹ [symbol] x² is a single bond, x² [symbol] x³ is a double bond, and R^{2B} is absent.

Embodiment 7: The compound of Embodiment 1, wherein X¹ is NR^{A}, X² is CR^{2A}R^{2B}, X³ is NR^{C}, x¹ [symbol] x² is a single bond, x² [symbol] x³ is a double bond, and R^{2B} and R^{C} are absent.

Embodiment 8: The compound of Embodiment 1, wherein X¹ is CR^{1A}R^{1B}, X² is NR^{B}, X³ is NR^{C}, x¹ [symbol] x² is a single bond, and X² [symbol] x³ is a single bond, and R^{B} is absent.

Embodiment 9: The compound of Embodiment 1, wherein X¹ is CR^{1A}R^{1B}, X² is C═O, X³ is NR^{C}, x¹ [symbol] x² is a single bond, x² [symbol] x³ is a single bond.

Embodiment 10: The compound of Embodiment 1, wherein X¹ is CR^{1A} R^{1B}, X² is CR^{2A}R^{2B}, X³ is O, x¹ [symbol] x² is a single bond, x² [symbol] x³ is a single bond.

Embodiment 11: The compound of Embodiment 1, wherein X¹ is CR^{1A}R^{1B}, X² is CR^{2A}R^{2B}, X³ is O, x¹ [symbol] x² is a double bond, x² [symbol] x³ is a single bond, R^{1B} is absent, and R^{2B} is absent.

Embodiment 12: The compound ofany one of Embodiments 1-11, wherein X⁴ is CR⁴.

Embodiment 13: The compound of any one of Embodiments 1-11, wherein X⁴ is N.

Embodiment 14: The compound of any one of Embodiments 1-13, wherein X⁵ is CR.

Embodiment 15: The compound of any one of Embodiments 1-11, wherein X⁵ is N.

Embodiment 16: The compound of any one of Embodiments 1-15, wherein X⁶ is CR⁶.

Embodiment 17: The compound of any one of Embodiments 1-11, wherein X⁶ is N.

Embodiment 18: The compound of any one of Embodiments 1-17, wherein X⁷ is CH.

Embodiment 19: The compound of any one of Embodiments 1-17, wherein X⁷ is CF.

Embodiment 20: The compound of any one of Embodiments 1-11, wherein X⁷ is N.

Embodiment 21: The compound of any one of Embodiments 1-11, wherein one of X⁴, X⁵, X⁶, and X⁷ are N.

Embodiment 22: The compound of any one of Embodiments 1-11, wherein two of X⁴, X⁵, X⁶, and X⁷ are N.

Embodiment 23: The compound of any one of Embodiments 1-11, wherein X⁴ is CR⁴; X⁵ is CR⁵; X⁶ is CR⁶; and X⁷ is CH.

V24: The compound of any one of Embodiments 1, 2, or 8-23, wherein R^{1A} is C1-C6 alkyl.

Embodiment 25: The compound of any one of Embodiments 1, 2, or 8-24, wherein R^{1A} is methyl.

Embodiment 26: The compound of any one of Embodiments 1, 2, or 8-23, wherein R^{1A} is —(C0-C6 alkyl)-5-6 membered heteroaryl.

Embodiment 27: The compound of any one of Embodiments 1, 2, 8-23, or 26, wherein R^{1A} is —(CH₂)-5-6 membered heteroaryl.

Embodiment 28: The compound of any one of Embodiments 1, 2, 8-23, wherein R^{1A} is —(C0 alkyl)-5-6 membered heteroaryl.

Embodiment 29: The compound of any one of Embodiments 1, 2, 8-23, or 28, wherein R^{1A} is

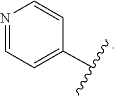 [SMILES: *c1ccncc1]

Embodiment 30: The compound of any one of Embodiments 1, 2, or 8-23, wherein R^{1A} is —C(═O)NR^{F}R^{G}.

Embodiment 31: The compound of any one of Embodiments 1, 2, or 8-23, wherein R^{1A} is —C(═O)-3-6 membered heterocyclyl optionally substituted with C1-C6 alkyl.

Embodiment 32: The compound of any one of Embodiments 1, 2, 8-23, or 31, wherein R^{1A} is —C(═O)-4-6 membered heterocyclyl optionally substituted with C1-C3 alkyl.

Embodiment 33: The compound of any one of Embodiments 1, 2, 8-23, or 31-32, wherein R^{1A} is —C(═O)-4-6 membered heterocyclyl substituted with C1-C3 alkyl.

Embodiment 34: The compound of any one of Embodiments 1, 2, 8-23, or 31, wherein R^{1A} is an unsubstituted —C(═O)-4-6 membered heterocyclyl.

Embodiment 35: The compound of any one of Embodiments 1, 2, 8-23, or 31-34, wherein R^{1A} is

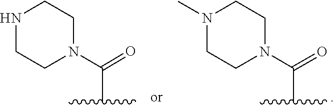

Embodiment 36: The compound of any one of Embodiments 1, 2, or 8-23, wherein R^{1A} is phenyl optionally substituted with halogen or —CO₂H.

Embodiment 37: The compound of any one of Embodiments 1, 2, 8-23, or 36, wherein R^{1A} is phenyl substituted with halogen.

Embodiment 38: The compound of any one of Embodiments 1, 2, 8-23, or 36, wherein R^{1A} is phenyl substituted with —CO₂H.

Embodiment 39: The compound of any one of Embodiments 1, 2, 8-23, or 36, wherein R^{1A} is unsubstituted phenyl.

Embodiment 40: The compound of any one of Embodiments 1, 2, or 8-23, wherein R^{1A} is 3-6 membered heterocyclyl optionally substituted with C1-C6 alkyl or —S(O₂)—C1-C6 alkyl.

Embodiment 41: The compound of any one of Embodiments 1, 2, 8-23, or 40 wherein R^{1A} is 3-6 membered heterocyclyl substituted with C1-C6 alkyl or —S(O₂)CH₃.

Embodiment 42: The compound of any one of Embodiments 1, 2, 8-23, or 40, wherein R^{1A} is an unsubstituted 3-6 membered heterocyclyl.

Embodiment 43: The compound of any one of Embodiments 1, 2, 8-23, or 40-42 wherein R is

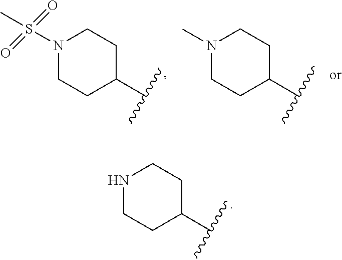

Embodiment 44: The compound of any one of Embodiments 1, 2, or 8-23, wherein R^{1A} is a C3-C6 cycloalkyl optionally substituted with hydroxyl, —C(═O)NR^{F}R^{G}, —NR^{F}R^{G}, —CO₂R^{H}, or C1-C6 alkoxy.

Embodiment 45: The compound of any one of Embodiments 1, 2, 8-23, or 44, wherein R^{1A} is a C3-C6 cycloalkyl substituted with hydroxyl, —C(═O)NR^{F}R^{G}, —NR^{F}R^{G}, —CO₂R, or C1-C6 alkoxy.

Embodiment 46: The compound of any one of Embodiments 1, 2, 8-23, or 44, wherein R^{1A} is an unsubstituted C3-C6 cycloalkyl.

Embodiment 47: The compound of any one of Embodiments 1, 2, 8-23, or 44-46 wherein R^{1A} is

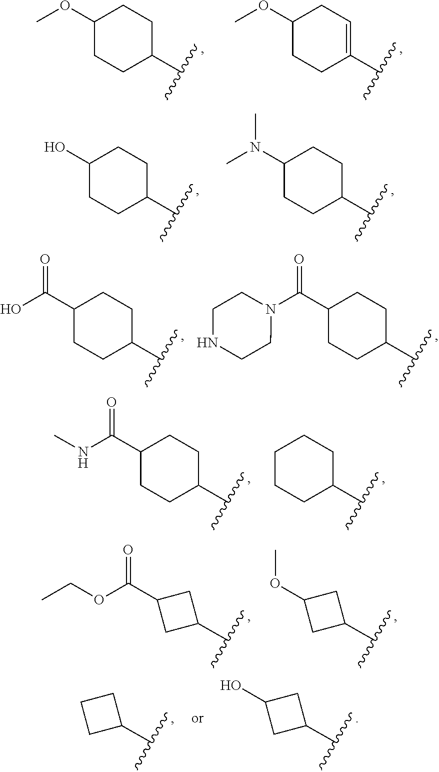

Embodiment 48: The compound of any one of Embodiments 1, 2, or 8-23, wherein R^{1A} is hydrogen.

Embodiment 49: The compound of any one of Embodiments 1 or 8-48, wherein R^{1B} is hydrogen.

Embodiment 50: The compound of any one of Embodiments 1, 2, or 8-48, wherein R^{1B} is absent.

Embodiment 51: The compound of any one of Embodiments 1-7 or 10-50, wherein R^{2A} is hydrogen.

Embodiment 52: The compound of any one of Embodiments 1-7 or 10-50, wherein R^{2A} is C1-C6 alkyl.

Embodiment 53: The compound of any one of Embodiments 1-7, 10-50, or 52, wherein R^{2A} is methyl.

Embodiment 54: The compound of any one of Embodiments 1-7 or 10-50, wherein R^{2A} is C1-C6 haloalkyl.

Embodiment 55: The compound of any one of Embodiments 1-7, 10-50, or 54 wherein R^{2A} is trifluoromethyl.

Embodiment 56: The compound of any one of Embodiments 1-7 or 10-50, wherein R^{2A} is C3-C6 cycloalkyl.

Embodiment 57: The compound of any one of Embodiments 1-7 or 10-50, wherein R^{2A} is 5-6 membered heteroaryl.

Embodiment 58: The compound of any one of Embodiments 1-7, 10-50, or 57 wherein R^{2A} is

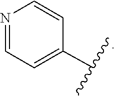 [SMILES: *c1ccncc1]

Embodiment 59: The compound of any one of Embodiments 1-7 or 10-50, wherein R^{2A} is phenyl optionally substituted with —CO₂H.

Embodiment 60: The compound of any one of Embodiments 1-7 or 10-50, wherein R^{2A} is phenyl substituted with —CO₂H.

Embodiment 61: The compound of any one of Embodiments 1-7 or 10-50, wherein R^{2A} is unsubstituted phenyl.

Embodiment 62: The compound of any one of Embodiments 1-7 or 10-50, wherein R^{2A} is a 3-6 membered heterocyclyl optionally substituted with C1-C6 alkyl, C1-C6 haloalkyl, C1-C6 hydroxyalkyl, or —(C0-C6 alkyl)-3-6 membered cycloalkyl optionally substituted with —CO₂H.

Embodiment 63: The compound of any one of Embodiments 1-7, 10-50, or 62, wherein R^{2A} is a 3-6 membered heterocyclyl substituted with C1-C6 alkyl, C1-C6 haloalkyl, C1-C6 hydroxyalkyl, or —(C0-C6 alkyl)-3-6 membered cycloalkyl optionally substituted with —CO₂H.

Embodiment 64: The compound of any one of Embodiments 1-7, 10-50, or 62, wherein R^{2A} is a 3-6 membered heterocyclyl substituted with C1-C6 alkyl, C1-C6 haloalkyl, C1-C6 hydroxyalkyl, or —(C0-C6 alkyl)-3-6 membered cycloalkyl substituted with —CO₂H.

Embodiment 65: The compound of any one of Embodiments 1-7, 10-50, or 62, wherein R^{2A} is a 3-6 membered heterocyclyl substituted with C1-C6 alkyl, C1-C6 haloalkyl, C1-C6 hydroxyalkyl, or an unsubstituted —(C0-C6 alkyl)-3-6 membered cycloalkyl.

Embodiment 66: The compound of any one of Embodiments 1-7, 10-50, or 62, wherein R^{2A} is an unsubstituted 3-6 membered heterocyclyl.

Embodiment 67: The compound of any one of Embodiments 1-7, 10-50 or 62-66, wherein R^{2A},

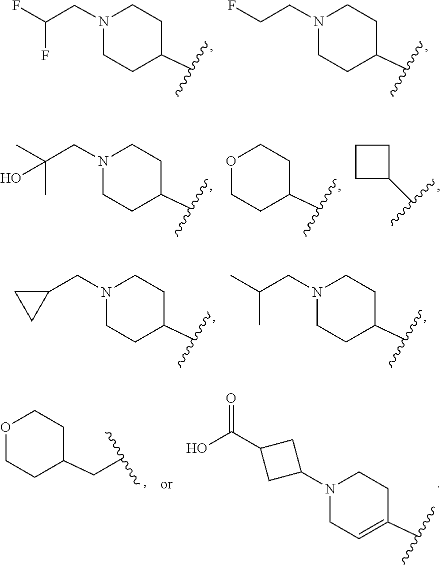

Embodiment 68: The compound of any one of Embodiments 1 or 10-67, wherein R^{2B} is hydrogen.

Embodiment 69: The compound of any one of Embodiments 1-7 or 10-67, wherein R^{2B} is absent.

Embodiment 70: The compound of any one of Embodiments 1, 5, or 12-69, wherein R^{1A} is hydrogen.

Embodiment 71: The compound of any one of Embodiments 1, 5, or 12-70, wherein R^{3B} is hydrogen.

Embodiment 72: The compound of any one of Embodiments 1, 5, or 12-70, wherein R^{3B} is C1-C6 alkyl.

Embodiment 73: The compound of any one of Embodiments 1, 5, 12-70, or 72, wherein R^{3B} is methyl.

Embodiment 74: The compound of any one of Embodiments 1, 5, or 12-70, wherein R^{3B} is absent.

Embodiment 75: The compound of any one of Embodiments 1-12 or 14-74, wherein R⁴ is hydrogen.

Embodiment 76: The compound of any one of Embodiments 1-12 or 14-74, wherein R⁴ is halogen.

Embodiment 77: The compound of any one of Embodiments 1-12 or 14-74, wherein R⁴ is C1-C6 alkyl.

Embodiment 78: The compound of any one of Embodiments 1-12, 14-74, or 77, wherein R⁴ is methyl.

Embodiment 79: The compound of any one of Embodiments 1-12 or 14-74, wherein R⁴ is 3-6 membered heterocyclyl optionally substituted with C1-C6 alkyl.

Embodiment 80: The compound of any one of Embodiments 1-12, 14-74, or 79, wherein R⁴ is 3-6 membered heterocyclyl substituted with C1-C6 alkyl.

Embodiment 81: The compound of any one of Embodiments 1-12, 14-74, or 79-80, wherein R⁴ is 3-6 membered heterocyclyl optionally substituted with methyl.

Embodiment 82: The compound of any one of Embodiments 1-12, 14-74, or 79, wherein R⁴ is an unsubstituted 3-6 membered heterocyclyl.

Embodiment 83: The compound of any one of Embodiments 1-12, 14-74, or 79-82, wherein R⁴ is

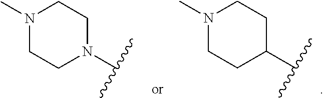

Embodiment 84: The compound of any one of Embodiments 1-14 or 16-83, wherein R⁵ is hydrogen.

Embodiment 85: The compound of any one of Embodiments 1-14 or 16-83, wherein R⁵ is —CO₂H or —C(═O)OCH₃.

Embodiment 86: The compound of any one of Embodiments 1-14 or 16-83, wherein R⁵ is C1-C6 alkyl optionally substituted with hydroxyl.

Embodiment 87: The compound of any one of Embodiments 1-14, 16-83, or 86, wherein R⁵ is C1-C6 alkyl substituted with hydroxyl.

Embodiment 88: The compound of any one of Embodiments 1-14, 16-83, or 86, wherein R⁵ is an unsubstituted C1-C6 alkyl.

Embodiment 89: The compound of any one of Embodiments 1-14 or 16-83, wherein R⁵ is a 5-6 membered heteroaryl optionally substituted with C1-C6 alkyl.

Embodiment 90: The compound of any one of Embodiments 1-14, 16-83, or 89, wherein R⁵ is a 5-6 membered heteroaryl substituted with C1-C6 alkyl.

Embodiment 91: The compound of any one of Embodiments 1-14, 16-83, or 89-90, wherein R⁵ is a 5-6 membered heteroaryl substituted with methyl.

Embodiment 92: The compound of any one of Embodiments 1-14, 16-83, or 89, wherein R⁵ is an unsubstituted 5-6 membered heteroaryl.

Embodiment 93: The compound of any one of Embodiments 1-14, 16-83, or 89-92, wherein R

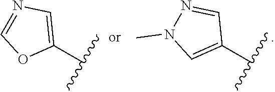

Embodiment 94: The compound of any one of Embodiments 1-16 or 18-93, wherein R⁶ is hydrogen.

Embodiment 95: The compound of any one of Embodiments 1-16 or 18-93, wherein R⁶ is a 5-6 membered heteroaryl optionally substituted with 1-2 substituents independently selected from R^{D}, C1-C6 alkyl, and 4-6 membered heterocyclyl optionally substituted with hydroxyl.

Embodiment 96: The compound of any one of Embodiments 1-16, 18-93, or 95, wherein R⁶ is a 5-6 membered heteroaryl substituted with 1-2 substituents independently selected from R^{D}, C1-C6 alkyl, and 4-6 membered heterocyclyl optionally substituted with hydroxyl.

Embodiment 97: The compound of any one of Embodiments 1-16, 18-93, or 95-96, wherein R⁶ is a 5-6 membered heteroaryl substituted with one substituent selected from R^{D}, C1-C6 alkyl, and a 4-6 membered heterocyclyl optionally substituted with hydroxyl.

Embodiment 98: The compound of any one of Embodiments 1-16, 18-93, or 95-96, wherein R⁶ is a 5-6 membered heteroaryl substituted with 2 substituents independently selected from R^{D}, C1-C6 alkyl, and 4-6 membered heterocyclyl optionally substituted with hydroxyl.

Embodiment 99: The compound of any one of Embodiments 1-16, 18-93, 95-96, or 98, wherein R⁶ is a 5-6 membered heteroaryl substituted with 1-2 substituents independently selected from R^{D}, C1-C6 alkyl, and 4-6 membered heterocyclyl substituted with hydroxyl.

Embodiment 100: The compound of any one of Embodiments 1-16, 18-93, 95-96, or 98-99, wherein R⁶ is a 5-6 membered heteroaryl substituted with 1-2 substituents independently selected from R^{D}, C1-C6 alkyl, and an unsubstituted 4-6 membered heterocyclyl.

Embodiment 101: The compound of any one of Embodiments 1-16, 18-93, or 95, wherein R⁶ is an unsubstituted 5-6 membered heteroaryl.

Embodiment 102: The compound of any one of Embodiments 1-16, 18-93, or 95-101, wherein R⁶ is

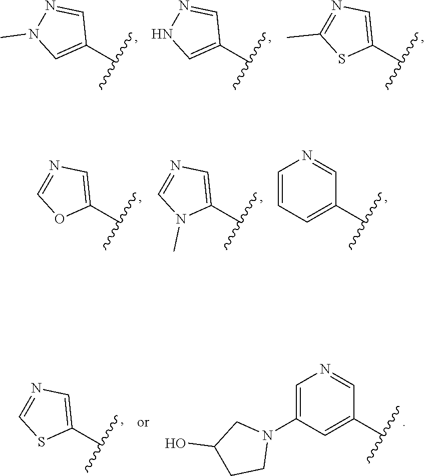

Embodiment 103: The compound of any one of Embodiments 1, 4, or 12-102, wherein R^{A} is absent.

Embodiment 104: The compound of any one of Embodiments 1, 3, 7, or 12-102, wherein R^{A} is hydrogen.

Embodiment 105: The compound of any one of Embodiments 1, 3, 7, or 12-102, wherein R^{A} is C1-C6 alkyl optionally substituted with 3-6 membered heterocyclyl or 5-6 membered heteroaryl.

Embodiment 106: The compound of any one of Embodiments 1, 3, 7, 12-102, or 105, wherein R^{A} is C1-C6 alkyl substituted with 3-6 membered heterocyclyl or 5-6 membered heteroaryl.

Embodiment 107: The compound of any one of Embodiments 1, 3, 7, 12-102, or 105-106, wherein R^{A} is C1-C6 alkyl substituted with 3-6 membered heterocyclyl.

Embodiment 108: The compound of any one of Embodiments 1, 3, 7, 12-102, or 105-106, wherein R^{A} is C1-C6 alkyl substituted with 5-6 membered heteroaryl.

Embodiment 109: The compound of any one of Embodiments 1, 3, 7, 12-102, or 105-108, wherein R^{A} is

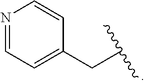 [SMILES: *Cc1ccncc1]

Embodiment 110: The compound of any one of Embodiments 1, 3, 7, or 12-102 wherein R^{A} is a C3-C6 cycloalkyl optionally substituted with hydroxyl or C1-C6 alkoxy.

Embodiment 111: The compound of any one of Embodiments 1, 3, 7, 12-102, or 110, wherein R^{A} is a C3-C6 cycloalkyl substituted with hydroxyl or C1-C6 alkoxy.

Embodiment 112: The compound of any one of Embodiments 1, 3, 7, 12-102, or 110, wherein R^{A} is an unsubstituted C3-C6 cycloalkyl.

Embodiment 113: The compound of any one of Embodiments 1, 3, 7, 12-102, or 110-112, wherein R^{A} is

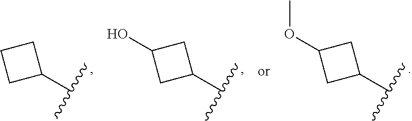

Embodiment 114: The compound of any one of Embodiments 1, 8, or 12-114, wherein R^{B} is absent.

Embodiment 115: The compound of any one of Embodiments 1, 8, or 12-114, wherein R^{B} is hydrogen.

Embodiment 116: The compound of any one of Embodiments 1, 8, or 12-114, wherein R^{B} is C1-C6 alkyl optionally substituted with 3-6 membered heterocyclyl or 5-6 membered heteroaryl.

Embodiment 117: The compound of any one of Embodiments 1, 8, 12-114, or 116, wherein R^{B} is C1-C6 alkyl substituted with 3-6 membered heterocyclyl or 5-6 membered heteroaryl.

Embodiment 118: The compound of any one of Embodiments 1, 8, 12-114, or 116-117, wherein R^{B} is C1-C6 alkyl substituted with 3-6 membered heterocyclyl.

Embodiment 119: The compound of any one of Embodiments 1, 8, 12-114, or 116-117, wherein R^{B} is C1-C6 alkyl substituted with 5-6 membered heteroaryl.

Embodiment 120: The compound of any one of Embodiments 1, 8, or 12-114, wherein R^{B} is a C3-C6 cycloalkyl optionally substituted with hydroxyl or C1-C6 alkoxy.

Embodiment 121: The compound of any one of Embodiments 1, 8, 12-114, or 120, wherein R^{B} is a C3-C6 cycloalkyl substituted with hydroxyl or C1-C6 alkoxy.

Embodiment 122: The compound of any one of Embodiments 1, 8, 12-114, or 120, wherein R^{B} is an unsubstituted C3-C6 cycloalkyl.

Embodiment 123: The compound of any one of Embodiments 1, 3, 6, 7, or 12-122, wherein R^{C} is absent.

Embodiment 124: The compound of any one of Embodiments 1, 4, 8, 9, or 12-122, wherein R^{C} is hydrogen.

Embodiment 125: The compound of any one of Embodiments 1, 4, 8, 9, or 12-122, wherein R^{C} is methyl.

Embodiment 126: The compound of any one of Embodiments 1-125, wherein R^{D} is

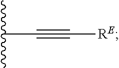

and R^{E} is a C1-C6 alkyl, phenyl, 5-6 membered heteroaryl, or 4-6 membered heterocyclyl; wherein the C1-C6 alkyl, phenyl, 5-6 membered heteroaryl, and 4-6 membered heterocyclyl are each optionally substituted with 1-2 independently selected R^{I}.

Embodiment 127: The compound of any one of Embodiments 1-126, wherein R^{E} is C1-C6 alkyl optionally substituted with 1-2 independently selected R^{I}.

Embodiment 128: The compound of any one of Embodiments 1-127, wherein R^{E} is C1-C6 alkyl optionally substituted with 1 R^{I}.

Embodiment 129: The compound of any one of Embodiments 1-127, wherein R^{E} is C1-C6 alkyl optionally substituted with 2 independently selected R^{I}.

Embodiment 130: The compound of any one of Embodiments 1-127, wherein R^{E} is an unsubstituted C1-C6 alkyl.

Embodiment 131: The compound of any one of Embodiments 1-126, wherein R^{E} is phenyl optionally substituted with 1-2 independently selected R^{I}.

Embodiment 132: The compound of any one of Embodiments 1-126 or 131, wherein R^{E} is phenyl optionally substituted with 1 R^{I}.

Embodiment 133: The compound of any one of Embodiments 1-126 or 131, wherein R^{E} is phenyl optionally substituted with 2 independently selected R^{I}.

Embodiment 134: The compound of any one of Embodiments 1-126 or 131, wherein R^{E} is an unsubstituted phenyl.

Embodiment 135: The compound of any one of Embodiments 1-126, wherein R^{E} is 5-6 membered heteroaryl optionally substituted with 1-2 independently selected R^{I}.

Embodiment 136: The compound of any one of Embodiments 1-126 or 135, wherein R^{E} is 5-6 membered heteroaryl optionally substituted with 1 R^{I}.

Embodiment 137: The compound of any one of Embodiments 1-126 or 135, wherein R^{E} is 5-6 membered heteroaryl optionally substituted with 2 independently selected R^{I}.

Embodiment 138: The compound of any one of Embodiments 1-126 or 135, wherein R^{E} is an unsubstituted 5-6 membered heteroaryl.

Embodiment 139: The compound of any one of Embodiments 1-126, wherein R^{E} is 4-6 membered heterocyclyl optionally substituted with 1-2 independently selected R^{I}.

Embodiment 140: The compound of any one of Embodiments 1-126 or 139, wherein R^{E} is 4-6 membered heterocyclyl optionally substituted with 1 R^{I}.

Embodiment 141: The compound of any one of Embodiments 1-126 or 139, wherein R^{E} is 4-6 membered heterocyclyl optionally substituted with 2 independently selected R^{I}.

Embodiment 142: The compound of any one of Embodiments 1-126 or 139, wherein R^{E} is an unsubstituted 4-6 membered heterocyclyl.

Embodiment 143: The compound of any one of Embodiments 1-126, wherein R^{D} is

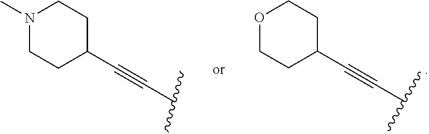

Embodiment 144: The compound of any one of Embodiments 1-23, 30, 44-45, or 49-143, wherein each R^{F} and R^{G} are independently selected from hydrogen and C1-C6 alkyl.

Embodiment 145: The compound of any one of Embodiments 1-23, 30, 44-45, or 49-143, wherein R^{F} and R^{G} together with the nitrogen atom to which they are attached form a 4-6 membered heterocyclyl optionally substituted with C1-C6 alkyl.

Embodiment 146: The compound of any one of Embodiments 1-23, 30, 44-45, 49-143, or 145, wherein R^{F} and R^{G} together with the nitrogen atom to which they are attached form

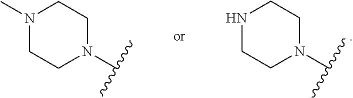

Embodiment 147: The compound of any one of Embodiments 1, 2, 8-23, 44-45, or 49-146, wherein R^{H} is hydrogen.

Embodiment 148: The compound of any one of Embodiments 1, 2, 8-23, 44-45, or 49-146, wherein R^{H} is C1-C6 alkyl.

Embodiment 149: The compound of any one of Embodiments 1-16, 18-129, 131-133, 135-137, 139-141, or 144-148, wherein R^{I} is C1-C6 alkyl, C1-C6 alkoxy, halogen, hydroxyl, cyano, or trifluoromethyl.

Embodiment 150: The compound of any one of Embodiments 1-16, 18-129, 131-133, 135-137, 139-141, or 144-149, wherein R^{I} is C1-C6 alkyl.

Embodiment 151: The compound of any one of Embodiments 1-16, 18-129, 131-133, 135-137, 139-141, or 144-149, wherein R^{I} is C1-C6 alkoxy.

Embodiment 152: The compound of any one of Embodiments 1-16, 18-129, 131-133, 135-137, 139-141, or 144-149, wherein R^{I} is halogen.

Embodiment 153: The compound of any one of Embodiments 1-16, 18-129, 131-133, 135-137, 139-141, or 144-149, wherein R^{I} is hydroxyl.

Embodiment 154: The compound of any one of Embodiments 1-16, 18-129, 131-133, 135-137, 139-141, or 144-149, wherein R^{I} is cyano.

Embodiment 155: The compound of any one of Embodiments 1-16, 18-129, 131-133, 135-137, 139-141, or 144-149, wherein R^{I} is trifluoromethyl.

Embodiment 156: The compound of any one of Embodiments 1-155, wherein the compound of Formula (I) has the structure of Formula (I-A):

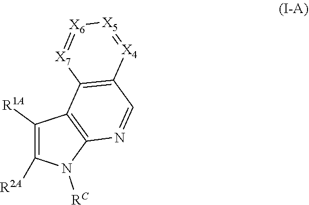

or a pharmaceutically acceptable salt thereof.

Embodiment 157: The compound of any one of Embodiments 1-155, wherein the compound of Formula (I) has the structure of Formula (I-B):

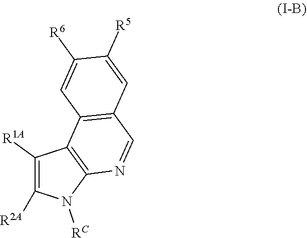

or a pharmaceutically acceptable salt thereof.

Embodiment 158: The compound of any one of Embodiments 1-155, wherein the compound of Formula (I) has the structure of Formula (I-C):

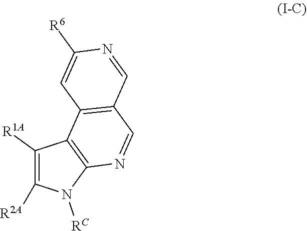

or a pharmaceutically acceptable salt thereof.

Embodiment 159: The compound of any one of Embodiments 1-155, wherein the compound of Formula (I) has the structure of Formula (I-D):

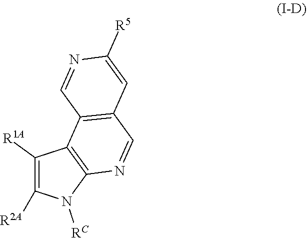

or a pharmaceutically acceptable salt thereof.

Embodiment 160: The compound of any one of Embodiments 1-155, wherein the compound of Formula (I) has the structure of Formula (I-E):

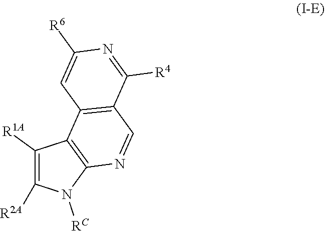

or a pharmaceutically acceptable salt thereof.

Embodiment 161: The compound of any one of Embodiments 1-155, wherein the compound of Formula (I) has the structure of Formula (I-F):

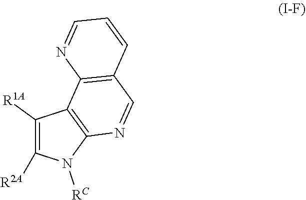

or a pharmaceutically acceptable salt thereof.

Embodiment 162: The compound of any one of Embodiments 1-155, wherein the compound of Formula (I) has the structure of Formula (I-G):

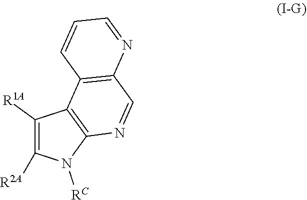

or a pharmaceutically acceptable salt thereof.

Embodiment 163: The compound of any one of Embodiments 1-155, wherein the compound of Formula (I) has the structure of Formula (I-H):

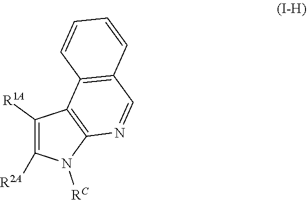

or a pharmaceutically acceptable salt thereof.

Embodiment 164: The compound of any one of Embodiments 1-155, wherein the compound of Formula (I) has the structure of Formula (I-I):

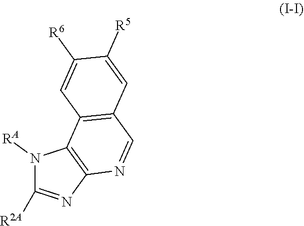

or a pharmaceutically acceptable salt thereof.

Embodiment 165: The compound of any one of Embodiments 1-155, wherein the compound of Formula (I) has the structure of Formula (I-J):

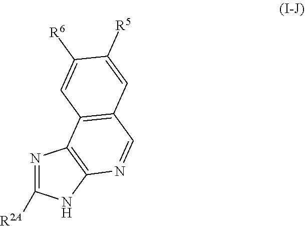

or a pharmaceutically acceptable salt thereof.

Embodiment 166: The compound of any one of Embodiments 1-155, wherein the compound of Formula (I) has the structure of Formula (I-K):

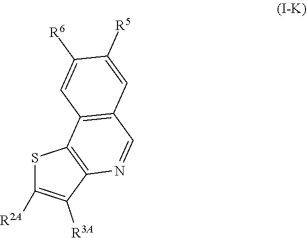

or a pharmaceutically acceptable salt thereof.

Embodiment 167: The compound of any one of Embodiments 1-155, wherein the compound of Formula (I) has the structure of Formula (I-L):

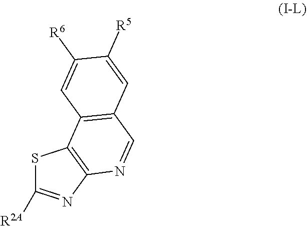

or a pharmaceutically acceptable salt thereof.

Embodiment 168: The compound of any one of Embodiments 1-155, wherein the compound of Formula (I) has the structure of Formula (I-M):

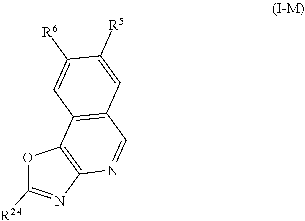

or a pharmaceutically acceptable salt thereof.

Embodiment 169: The compound of any one of Embodiments 1-155, wherein the compound of Formula (I) has the structure of Formula (I-N):

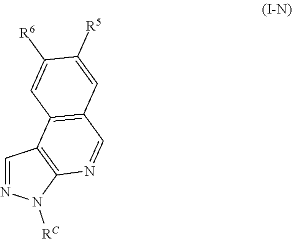

or a pharmaceutically acceptable salt thereof.

Embodiment 170: The compound of any one of Embodiments 1-155, wherein the compound of Formula (I) has the structure of Formula (1-0):

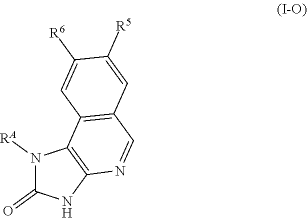

or a pharmaceutically acceptable salt thereof.

Embodiment 171: The compound of any one of Embodiments 1-155, wherein the compound of Formula (I) has the structure of Formula (I-P):

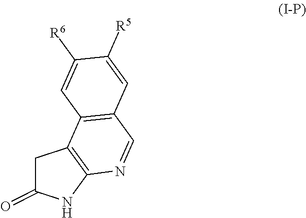

or a pharmaceutically acceptable salt thereof.

Embodiment 172: The compound of any one of Embodiments 1-155, wherein the compound of Formula (I) has the structure of Formula (I-Q):

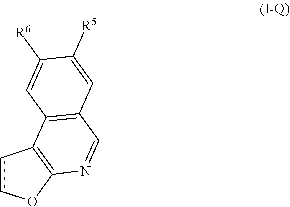

or a pharmaceutically acceptable salt thereof.

### Exemplary Embodiments of Compounds of Formula (II)

Embodiment 1: a compound of Formula (II):

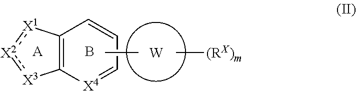

or a pharmaceutically acceptable salt thereof, wherein:
each dashed line represents a single bond or a double bond;
Rings A and B are aromatic;
Ring W is a 9 membered heteroaryl, a 9 membered heterocyclyl, or a 9 membered cycloalkyl;
each R^{X} is independently selected from C1-C6 alkyl, 3-6 membered heterocyclyl optionally substituted with C1-C6 alkyl, and C3-C6 cycloalkyl;
X¹ is CH, S, N, or NR^{A};
X² is N, CH, or CR²;
X³ is N, NR^{B}, O, CR³, or CH;
X⁴ is CH or N;
R² is benzyl or

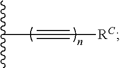

R³ is C1-C6 alkyl;
R^{A} is C1-C6 alkyl or C3-C6 cycloalkyl;
R^{B} is hydrogen or C3-C6 cycloalkyl;
R^{C} is 3-6 membered heterocyclyl optionally substituted with C1-C6 alkyl;
m is 0, 1, or 2; and
n is 0 or 1.

Embodiments 2: The compound of embodiment 1, wherein the dashed line between X¹ and X² represents a single bond and the dashed line between X² and X³ represents a double bond.

Embodiment 3: The compound of embodiment 1, wherein the dashed line between X¹ and X² represents a double bond and the dashed line between X² and X³ represents a single bond.

Embodiment 4: The compound of any one of embodiments 1-3, wherein Ring W is a 9 membered heteroaryl.

Embodiment 5: The compound of embodiment 4, wherein Ring W is pyrazolo[1,5-a]pyridinyl, pyrazolo[1,5-a]pyrimidinyl, 1H-pyrrolo[2,3-b]pyridinyl, 1H-pyrrolo[2,3-c]pyridinyl, thiazolo[4,5-c]pyridinyl, 1,3-dihydro-2H-imidazo[4,5-b]pyridin-2-only, indazolyl, or imidazo[1,2-a]pyrazine.

Embodiment 6: The compound of any one of embodiments 4-5, wherein Ring W is

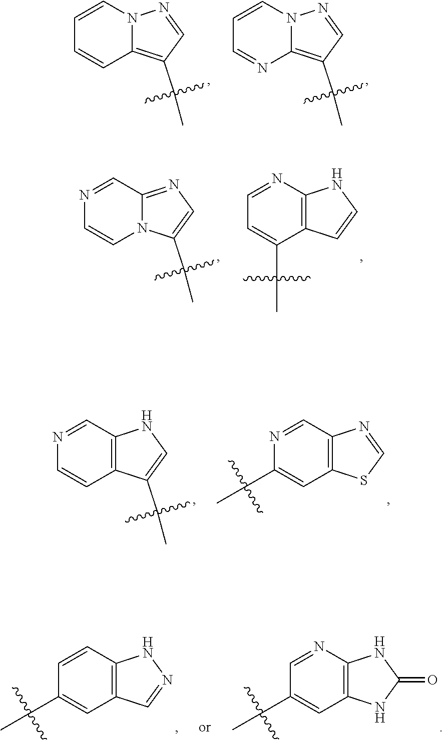

Embodiment 7: The compound of any one of embodiments 1-3, wherein Ring W is a 9 membered heterocyclyl.

Embodiment 8: The compound of embodiment 7, wherein Ring W is methylenedioxyphenyl.

Embodiment 9: The compound of embodiment 8, wherein Ring W is

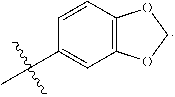 [SMILES: *c1ccc2c(c1)OCO2]

Embodiment 10: The compound of any one of embodiments 1-3, wherein Ring W is a 9 membered cycloalkyl.

Embodiment 11: The compound of any one of embodiments 1-10, wherein m is 1.

Embodiment 12: The compound of any one of embodiments 1-10, wherein m is 2.

Embodiment 13: The compound of any one of embodiments 1-12, wherein at least one R^{X} is C1-C6 alkyl.

Embodiment 14: The compound of embodiment 1-13, wherein at least one R^{X} is methyl.

Embodiment 15: The compound of any one of embodiments 1-14, wherein at least one R^{X} is 3-6 membered heterocyclyl optionally substituted with C1-C6 alkyl.

Embodiment 16: The compound of any one of embodiments 1-15, wherein at least one R^{X} is 3-6 membered heterocyclyl optionally substituted with methyl.

Embodiment 17: The compound of any one of embodiments 1-15, wherein at least one R^{X} is 3-6 membered heterocyclyl optionally substituted with ethyl.

Embodiment 18: The compound of any one of embodiments 1-15, wherein at least one R^{X} is 3-6 membered heterocyclyl optionally substituted with isobutyl.

Embodiment 19: The compound of any one of embodiments 1-15, wherein at least one R^{X} is 3-6 membered heterocyclyl substituted with C1-C6 alkyl.

Embodiment 20: The compound of any one of embodiments 1-15 and 19, wherein at least one R^{X} is 3-6 membered heterocyclyl substituted with methyl.

Embodiment 21: The compound of any one of embodiments 1-15 and 19, wherein at least one R^{X} is 3-6 membered heterocyclyl substituted with ethyl.

Embodiment 22: The compound of any one of embodiments 1-15 and 19, wherein at least one R^{X} is 3-6 membered heterocyclyl substituted with isobutyl.

Embodiment 23: The compound of any one of embodiments 1-15, wherein at least one R^{X} is unsubstituted 3-6 membered heterocyclyl.

Embodiment 24: The compound of any one of embodiments 15-23, wherein the R^{X} heteroaryl is a 5-6 membered heteroaryl.

Embodiment 25: The compound of any one of embodiments 15-24, wherein the R^{X} heteroaryl is a 6 membered heteroaryl.

Embodiment 26: The compound of any one of embodiments 15-25, wherein the R^{X} heteroaryl is a pyridonyl.

Embodiment 27: The compound of any one of embodiments 1-12, wherein at least one R^{X} is C3-C6 cycloalkyl.

Embodiment 28: The compound of any one of embodiments 1-12 and 27, wherein at least one R^{X} is C3-C5 cycloalkyl.

Embodiment 29: The compound of any one of embodiments 1-12 and 27-28, wherein at least one R^{X} is C3-C4 cycloalkyl.

Embodiment 30: The compound of any one of embodiments 1-12 and 27-29, wherein at least one R^{X} is cyclobutyl.

Embodiment 31: The compound of any one of embodiments 1-10, wherein m is O.

Embodiment 32: The compound of any one of embodiments 1-31, wherein X¹ is CH.

Embodiment 33: The compound of any one of embodiments 1-31, wherein X¹ is S.

Embodiment 34: The compound of any one of embodiments 1-31, wherein X¹ is N.

Embodiment 35: The compound of any one of embodiments 1-31, wherein X¹ is NR^{A}.

Embodiment 36: The compound of any one of embodiments 1-31 and 35, wherein R^{A} is C1-C6 alkyl.

Embodiment 37: The compound of any one of embodiments 1-31 and 35, wherein R^{A} is C1-C3 alkyl.

Embodiment 38: The compound of any one of embodiments 1-31 and 35, wherein R^{A} is methyl.

Embodiment 39: The compound of any one of embodiments 1-31 and 35, wherein R^{A} is ethyl.

Embodiment 40: The compound of any one of embodiments 1-31 and 35, wherein R^{A} is isopropyl.

Embodiment 41: The compound of any one of embodiments 1-31 and 35, wherein R^{A} is C3-C6 cycloalkyl.

Embodiment 42: The compound of any one of embodiments 1-31 and 35, wherein R^{A} is C3-C4 cycloalkyl.

Embodiment 43: The compound of any one of embodiments 1-31 and 35, wherein R^{A} is cyclopropyl.

Embodiment 44: The compound of any one of embodiments 1-31 and 35, wherein R^{A} is cyclobutyl.

Embodiment 45: The compound of any one of embodiments 1-44, wherein X² is N.

Embodiment 46: The compound of any one of embodiments 1-44, wherein X² is CH.

Embodiment 47: The compound of any one of embodiments 1-44, wherein X² is CR² Embodiment 48: The compound of any one of embodiments 1-44 and 47, wherein R² is benzyl.

Embodiment 49: The compound of any one of embodiments 1-44 and 47, wherein R² is

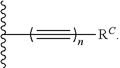

Embodiment 50: The compound of any one of embodiments 1-44 and 47, wherein n is 0.

Embodiment 51: The compound of any one of embodiments 1-44 and 47, wherein n is 1.

Embodiment 52: The compound of any one of embodiments 1-44, 47, and 49-51, wherein R^{C} is 3-6 membered heterocyclyl optionally substituted with methyl.

Embodiment 53: The compound of any one of embodiments 1-44, 47, and 49-51, wherein R^{C} is 3-6 membered heterocyclyl optionally substituted with isobutyl.

Embodiment 54: The compound of any one of embodiments 1-44, 47, and 49-51, wherein R^{C} is 3-6 membered heterocyclyl substituted with C1-C6 alkyl.

Embodiment 55: The compound of any one of embodiments 1-44, 47, and 49-51, wherein R^{C} is 3-6 membered heterocyclyl substituted with methyl.

Embodiment 56: The compound of any one of embodiments 1-44, 47, and 49-51, wherein R^{C} is 3-6 membered heterocyclyl substituted with isobutyl.

Embodiment 57: The compound of any one of embodiments 1-44, 47, and 49-51, wherein R^{C} is unsubstituted 3-6 membered heterocyclyl.

Embodiment 58: The compound of any one of embodiments 52-57, wherein the R^{C} heterocyclyl is a 5-6 membered heterocyclyl.

Embodiment 59: The compound of any one of embodiments 52-57, wherein the R^{C} heterocyclyl is a piperidinyl.

Embodiment 60: The compound of any one of embodiments 52-57, wherein the R^{C} heterocyclyl is a 1-piperidinyl.

Embodiment 61: The compound of any one of embodiments 1-60, wherein X³ is N.

Embodiment 62: The compound of any one of embodiments 1-60, wherein X³ is NR^{B}.

Embodiment 63: The compound of any one of embodiments 1-60 and 62, wherein R^{B} is hydrogen.

Embodiment 64: The compound of any one of embodiments 1-60 and 62, wherein R^{B} is C3-C6 cycloalkyl.

Embodiment 65: The compound of any one of embodiments 1-60 and 62, wherein R^{B} is C3-C4 cycloalkyl.

Embodiment 66: The compound of any one of embodiments 1-60 and 62, wherein R^{B} is cyclopropyl.

Embodiment 67: The compound of any one of embodiments 1-60 and 62, wherein R^{B} is cyclobutyl.

Embodiment 68: The compound of any one of embodiments 1-60, wherein X³ is O.

Embodiment 69: The compound of any one of embodiments 1-60, wherein X³ is CR³.

Embodiment 70: The compound of any one of embodiments 1-60 and 69, wherein R³ is C1-C6 alkyl.

Embodiment 71: The compound of any one of embodiments 1-60 and 69, wherein R³ is methyl.

Embodiment 72: The compound of any one of embodiments 1-60, wherein X³ is CH.

Embodiment 73: The compound of any one of embodiments 1-72, wherein X⁴ is CH.

Embodiment 74: The compound of any one of embodiments 1-72, wherein X⁴ is N.

Embodiment 75: The compound of embodiment 1, wherein:
X⁵ is CH, CR⁵, or N;
X⁶ is CH or N;
X⁷ is N or C;
X⁸ is N, CH, or NR^{D};
X⁹ is CH, NH, or N;
X¹⁰ is CH, CH₂, or N;
X¹¹ is CH or N;
R⁵ is 3-6 membered heterocyclyl optionally substituted with C1-C6 alkyl; and
R^{D} is C1-C6 alkyl or C3-C6 cycloalkyl.

Embodiment 76: The compound of embodiment 1, wherein the compound is a compound of Formula (II-A):

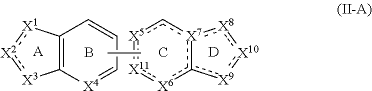

wherein:
X¹, X², X³, and X⁴ are as defined in embodiment 1;
Ring C is aromatic;
X⁵ is CH, CR⁵, or N;
X⁶ is CH or N;
X⁷ is N or C;
X⁸ is N, O, CH, or NR^{D};
X⁹ is CH, NH, N, O, or S;
X¹⁰ is CH, CH₂, C(═O), or N;
X¹¹ is CH or N;
R⁵ is 3-6 membered heterocyclyl optionally substituted with C1-C6 alkyl; and
R^{D} is C1-C6 alkyl or C3-C6 cycloalkyl.

Embodiment 77: The compound of embodiment 1, wherein the compound is a compound of Formula (II-A-i):

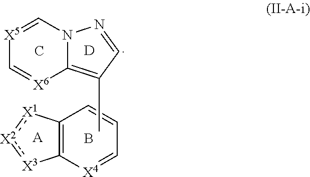

Embodiment 78: The compound of embodiment 1, wherein the compound is a compound of Formula (II-A-ii):

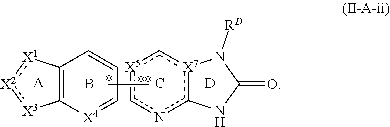

Embodiment 79: The compound of embodiment 1, wherein the compound is a compound of Formula (II-A-iii):

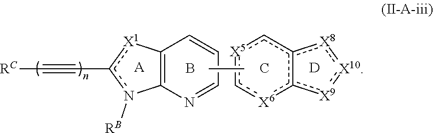

Embodiment 80: The compound of embodiment 1, wherein the compound is a compound of Formula (II-A-iv):

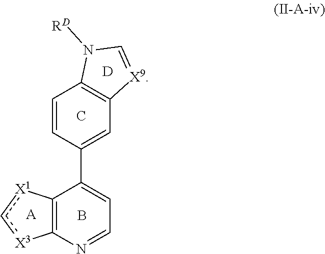

Embodiment 81: The compound of any one of embodiments 76-78, wherein the dashed line between X¹ and X² represents a single bond and the dashed line between X² and X³ represents a double bond.

Embodiment 82: The compound of any one of embodiments 76-78, wherein the dashed line between X¹ and X² represents a double bond and the dashed line between X² and X³ represents a single bond.

Embodiment 83: The compound of any one of embodiments 76-82, wherein X¹ is CH.

Embodiment 84: The compound of any one of embodiments 76-82, wherein X¹ is S.

Embodiment 85: The compound of any one of embodiments 76-82, wherein X¹ is N.

Embodiment 86: The compound of any one of embodiments 76-82, wherein X¹ is NR^{A}.

Embodiment 87: The compound of any one of embodiments 76-82 and 86, wherein R^{A} is C1-C6 alkyl.

Embodiment 88: The compound of any one of embodiments 76-82 and 86, wherein R^{A} is C1-C3 alkyl.

Embodiment 89: The compound of any one of embodiments 76-82 and 86, wherein R^{A} is methyl.

Embodiment 90: The compound of any one of embodiments 76-82 and 86, wherein R^{A} is ethyl.

Embodiment 91: The compound of any one of embodiments 76-82 and 86, wherein R^{A} is isopropyl.

Embodiment 92: The compound of any one of embodiments 76-82 and 86, wherein R^{A} is C3-C6 cycloalkyl.

Embodiment 93: The compound of any one of embodiments 76-82 and 86, wherein R^{A} is C3-C4 cycloalkyl.

Embodiment 94: The compound of any one of embodiments 76-82 and 86, wherein R^{A} is cyclopropyl.

Embodiment 95: The compound of any one of embodiments 76-82 and 86, wherein R^{A} is cyclobutyl.

Embodiment 96: The compound of any one of embodiments 76-78 and 81-95, wherein X² is N.

Embodiment 97: The compound of any one of embodiments 76-78 and 81-95, wherein X² is CH.

Embodiment 98: The compound of any one of embodiments 76-78 and 81-95, wherein X² is CR².

Embodiment 99: The compound of any one of embodiments 76-78, 81-95, and 98, wherein R² is benzyl.

Embodiment 100: The compound of any one of embodiments 76-78, 81-95, and 98, R^{C} wherein R² is

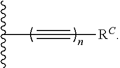

Embodiment 101: The compound of any one of embodiments 76-79, 81-95, and 98, wherein n is 0.

Embodiment 102: The compound of any one of embodiments 76-79, 81-95, and 98, wherein n is 1.

Embodiment 103: The compound of any one of embodiments 76-79, 81-95, 98, and 100-102, wherein R^{C} is 3-6 membered heterocyclyl optionally substituted with methyl.

Embodiment 104: The compound of any one of embodiments 76-79, 81-95, 98, and 100-102, wherein R^{C} is 3-6 membered heterocyclyl optionally substituted with isobutyl.

Embodiment 105: The compound of any one of embodiments 76-79, 81-95, 98, and 100-102 wherein R^{C} is 3-6 membered heterocyclyl substituted with C1-C6 alkyl.

Embodiment 106: The compound of any one of embodiments 76-79, 81-95, 98, and 100-102, wherein R^{C} is 3-6 membered heterocyclyl substituted with methyl.

Embodiment 107: The compound of any one of embodiments 76-79, 81-95, 98, and 100-102, wherein R^{C} is 3-6 membered heterocyclyl substituted with isobutyl.

Embodiment 108: The compound of any one of embodiments 76-79, 81-95, 98, and 100-102, wherein R^{C} is unsubstituted 3-6 membered heterocyclyl.

Embodiment 109: The compound of any one of embodiments 104-108, wherein the R^{C} heterocyclyl is a 5-6 membered heterocyclyl.

Embodiment 110: The compound of any one of embodiments 104-109, wherein the R^{C} heterocyclyl is a piperidinyl.

Embodiment 111: The compound of any one of embodiments 104-109, wherein the R^{C} heterocyclyl is a 1-piperidinyl.

Embodiment 112: The compound of any one of embodiments 76-78 and 80-111, wherein X³ is N.

Embodiment 113: The compound of any one of embodiments 76-78 and 80-111, wherein X³ is NR^{B}.

Embodiment 114: The compound of any one of embodiments 76-111 and 113, wherein R^{B} is hydrogen.

Embodiment 115: The compound of any one of embodiments 76-111 and 113, wherein R^{B} is C3-C6 cycloalkyl.

Embodiment 116: The compound of any one of embodiments 76-111 and 113, wherein R^{B} is C3-C4 cycloalkyl.

Embodiment 117: The compound of any one of embodiments 76-111 and 113, wherein R^{B} is cyclopropyl.

Embodiment 118: The compound of any one of embodiments 76-111 and 113, wherein R^{B} is cyclobutyl.

Embodiment 119: The compound of any one of embodiments 76-78 and 80-111, wherein X³ is O.

Embodiment 120: The compound of any one of embodiments 76-78 and 80-111, wherein X³ is CR³.

Embodiment 121: The compound of any one of embodiments 76-78 and 80-111 and 120, wherein R³ is C1-C6 alkyl.

Embodiment 122: The compound of any one of embodiments 76-78 and 80-111 and 120, wherein R³ is methyl.

Embodiment 123: The compound of any one of embodiments 76-78 and 80-111, wherein X³ is CH.

Embodiment 124: The compound of any one of embodiments 76-78 and 81-123, wherein X⁴ is CH.

Embodiment 125: The compound of any one of embodiments 76-78 and 81-123, wherein X⁴ is N.

Embodiment 126: The compound of embodiment 1, wherein:
X⁵ is CH, CR⁵, or N;
X⁶ is CH or N;
X⁷ is N or C;
X⁸ is N, CH, or NR^{D};
X⁹ is CH, NH, or N;
X¹⁰ is CH, CH₂, or N;
X¹¹ is CH or N;
R⁵ is 3-6 membered heterocyclyl optionally substituted with C1-C6 alkyl; and
R^{D} is C1-C6 alkyl or C3-C6 cycloalkyl.

Embodiment 127: The compound of any one of embodiments 76-79 and 81-126, wherein X⁵ is CH.

Embodiment 128: The compound of any one of embodiments 76-79 and 81-126, wherein X⁵ is CR⁵.

Embodiment 129: The compound of any one of embodiments 76-79, 81-126, and 128, wherein R⁵ is 3-6 membered heterocyclyl optionally substituted with C1-C6 alkyl.

Embodiment 130: The compound of any one of embodiments 76-79, 81-126, and 129, wherein R⁵ is 3-6 membered heterocyclyl optionally substituted with methyl.

Embodiment 131: The compound of any one of embodiments 76-79, 81-126, and 129, wherein R⁵ is 3-6 membered heterocyclyl optionally substituted with ethyl.

Embodiment 132: The compound of any one of embodiments 76-79, 81-126, and 129, wherein R⁵ is 3-6 membered heterocyclyl optionally substituted with isobutyl.

Embodiment 133: The compound of any one of embodiments 76-79, 81-126, and 129, wherein R⁵ is 3-6 membered heterocyclyl substituted with C1-C6 alkyl.

Embodiment 134: The compound of any one of embodiments 76-79, 81-126, and 129, wherein R⁵ is 3-6 membered heterocyclyl substituted with methyl.

Embodiment 135: The compound of any one of embodiments 76-79, 81-126, and 129, wherein R⁵ is 3-6 membered heterocyclyl substituted with ethyl.

Embodiment 136: The compound of any one of embodiments 76-79, 81-126, and 129, wherein R⁵ is 3-6 membered heterocyclyl substituted with isobutyl.

Embodiment 137: The compound of any one of embodiments 76-79, 81-126, and 129, wherein R⁵ is unsubstituted 3-6 membered heterocyclyl.

Embodiment 138: The compound of any one of embodiments 129-137, wherein the R⁵ heteroaryl is a 5-6 membered heteroaryl.

Embodiment 139: The compound of any one of embodiments 129-137, wherein the R⁵ heteroaryl is a 6 membered heteroaryl.

Embodiment 140: The compound of any one of embodiments 129-137, wherein the R⁵ heteroaryl is a pyridonyl.

Embodiment 141: The compound of any one of embodiments 76-79 and 81-126, wherein X⁵ is N.

Embodiment 142: The compound of any one of embodiments 76-77, 79. and 81-141, wherein X⁶ is CH.

Embodiment 143: The compound of any one of embodiments 76-77, 79. and 81-141, wherein X⁶ is N.

Embodiment 144: The compound of any one of embodiments 76, 78, 81-143, wherein X⁷ is N.

Embodiment 145: The compound of any one of embodiments 76, 78, 81-143, wherein X⁷ is C.

Embodiment 146: The compound of any one of embodiments 76, 79, and 81-145, wherein X⁸ is N.

Embodiment 147: The compound of any one of embodiments 76, 79, and 81-145, wherein X⁸ is O.

Embodiment 148: The compound of any one of embodiments 76, 79, and 81-145, wherein X⁸ is CH.

Embodiment 149: The compound of any one of embodiments 76, 79, and 81-145, wherein X⁸ is NR^{D}.

Embodiment 150: The compound of any one of embodiments 76, 78-80, 81-145, and 149, wherein R^{D} is C1-C6 alkyl.

Embodiment 151: The compound of any one of embodiments 76, 78-80, 81-145, and 149, wherein R^{D} is methyl.

Embodiment 152: The compound of any one of embodiments 76, 78-80, 81-145, and 149, wherein R^{D} is C3-C6 cycloalkyl.

Embodiment 153: The compound of any one of embodiments 76, 78-80, 81-145, and 149, wherein R^{D} is C3-C5 cycloalkyl.

Embodiment 154: The compound of any one of embodiments 76, 78-80, 81-145, and 149, wherein R^{D} is C3-C4 cycloalkyl.

Embodiment 155: The compound of any one of embodiments 76, 78-80, 81-146, and 149, wherein R^{D} is cyclobutyl.

Embodiment 156: The compound of any one of embodiments 76, 79-80, and 81-155, wherein X⁹ is CH.

Embodiment 157: The compound of any one of embodiments 76, 79-80, and 81-155, wherein X⁹ is NH.

Embodiment 158: The compound of any one of embodiments 76, 79-80, and 81-155, wherein X⁹ is N.

Embodiment 159: The compound of any one of embodiments 76, 79-80, and 81-155, wherein X⁹ is O.

Embodiment 160: The compound of any one of embodiments 76, 79-80, and 81-155, wherein X⁹ is S.

Embodiment 161: The compound of any one of embodiments 76, 79, and 81-160, wherein X¹⁰ is CH.

Embodiment 162: The compound of any one of embodiments 76, 79, and 81-160, wherein X¹⁰ is CH₂.

Embodiment 163: The compound of any one of embodiments 76, 79, and 81-160, wherein X¹⁰ is C(═O).

Embodiment 164: The compound of any one of embodiments 76, 79, and 81-160, wherein X¹⁰ is N.

Embodiment 165: The compound of any one of embodiments 76 and 81-164, wherein X¹¹ is CH.

Embodiment 166: The compound of any one of embodiments 76 and 81-164, wherein X¹¹ is N.

### Exemplary Embodiments of Compounds of Formula (III)

Embodiment 1: A compound of Formula (III):

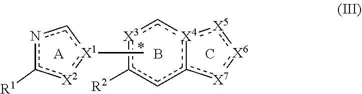

or a pharmaceutically acceptable salt thereof, wherein:
each dashed line represents a single bond or a double bond;
Rings A and B are aromatic;
Ring C is aromatic or partially saturated;
X¹ is C or N;
X² is CH, O, or S;
X³ is CH or N;
X⁴ is C or N;
X⁵ is CH, C(═O), O, S, NH, or NR^{A};
X⁶ is CH, N, or O;
X⁷ is CH, CR⁷, CH₂, CR^{B}R^{C}, or N;
R¹ is hydrogen or —XR^{D};
R² is hydrogen, —NHC(═O)-3-6 membered heterocyclyl optionally substituted with C1-C6 haloalkyl, or joins with the bond denoted with *;
R⁷ is C1-C6 alkyl optionally substituted with a 3-6 membered heterocyclyl optionally substituted with C(═O)OH;
R^{A} is -(3-6 membered heterocyclyl)ₘ-(C1-C6 alkyl)ₙ;
R^{B} and R^{C} are independently hydrogen or C1-C6 alkyl;
X is ethynylene, —NHC(═O)—, or —NHC(═O)OCH₂—;
R^{D} is 3-6 membered heterocyclyl optionally substituted with C1-C6 alkyl, 5-6 membered heteroaryl optionally substituted with C1-C6 alkyl, or C6-C10 aryl optionally substituted with C1-C6 alkyl;
m is 0 or 1; and
n is 0, 1, or 2;
provided that when n is 2, m is 1.

Embodiment 2: The compound of embodiment 1, wherein R¹ is hydrogen.

Embodiment 3: The compound of embodiment 1, wherein R¹ is —XR^{D}.

Embodiment 4: The compound of any one of embodiments 1 and 3, wherein X is ethynylene.

Embodiment 5: The compound of any one of embodiments 1 and 3, wherein X is —NHC(═O)—.

Embodiment 6: The compound of any one of embodiments 1 and 3, wherein X is —NHC(═O)OCH₂—.

Embodiment 7: The compound of any one of embodiments 1 and 3-6, wherein R^{D} is 3-6 membered heterocyclyl optionally substituted with C1-C6 alkyl.

Embodiment 8: The compound of any one of embodiments 1 and 3-6, wherein R^{D} is 3-6 membered heterocyclyl optionally substituted with methyl.

Embodiment 9: The compound of any one of embodiments 1 and 3-6, wherein R^{D} is 3-6 membered heterocyclyl substituted with C1-C6 alkyl.

Embodiment 10: The compound of any one of embodiments 1 and 3-6, wherein R^{D} is 3-6 membered heterocyclyl substituted with methyl.

Embodiment 11: The compound of any one of embodiments 1 and 3-6, wherein R^{D} is unsubstituted 3-6 membered heterocyclyl.

Embodiment 12: The compound of any one of embodiments 7-11, wherein the R^{D} 3-6 membered heterocyclyl is 5-6 membered heterocyclyl.

Embodiment 13: The compound of any one of embodiments 7-11, wherein the R^{D} 3-6 membered heterocyclyl is piperidinyl.

Embodiment 14: The compound of any one of embodiments 1 and 3-6, wherein R^{D} is 5-6 membered heteroaryl optionally substituted with C1-C6 alkyl.

Embodiment 15: The compound of any one of embodiments 1 and 3-6, wherein R^{D} is 5-6 membered heteroaryl optionally substituted with methyl.

Embodiment 16: The compound of any one of embodiments 1 and 3-6, wherein R^{D} is 5-6 membered heteroaryl substituted with C1-C6 alkyl.

Embodiment 17: The compound of any one of embodiments 1 and 3-6, wherein R^{D} is 5-6 membered heteroaryl substituted with methyl.

Embodiment 18: The compound of any one of embodiments 1 and 3-6, wherein R^{D} is unsubstituted 5-6 membered heteroaryl.

Embodiment 19: The compound of any one of embodiments 14-18, wherein the R^{D} 5-6 membered heteroaryl is 6 membered heteroaryl.

Embodiment 20: The compound of any one of embodiments 14-18, wherein the R^{D} 5-6 membered heteroaryl is pyridonyl.

Embodiment 21: The compound of any one of embodiments 1 and 3-6, wherein R^{D} is C6-C10 aryl optionally substituted with C1-C6 alkyl.

Embodiment 22: The compound of any one of embodiments 1 and 3-6, wherein R^{D} is C6-C10 aryl optionally substituted with methyl.

Embodiment 23: The compound of any one of embodiments 1 and 3-6, wherein R^{D} is C6-C10 aryl substituted with C1-C6 alkyl.

Embodiment 24: The compound of any one of embodiments 1 and 3-6, wherein R^{D} is C6-C10 aryl substituted with methyl.

Embodiment 25: The compound of any one of embodiments 1 and 3-6, wherein R^{D} is unsubstituted C6-C10 aryl.

Embodiment 26: The compound of any one of embodiments 21-25, wherein the R^{D} C6-C10 aryl is phenyl.

Embodiment 27: The compound of any one of embodiments 1-26, wherein R² is hydrogen.

Embodiment 28: The compound of any one of embodiments 1-26, wherein R² is —NHC(═O)-3-6 membered heterocyclyl optionally substituted with C1-C6 haloalkyl.

Embodiment 29: The compound of any one of embodiments 1-26, wherein R² is —NHC(═O)-3-6 membered heterocyclyl optionally substituted with fluoroethyl.

Embodiment 30: The compound of any one of embodiments 1-26, wherein R² is —NHC(═O)-3-6 membered heterocyclyl substituted with C1-C6 haloalkyl.

Embodiment 31: The compound of any one of embodiments 1-26, wherein R² is —NHC(═O)-3-6 membered heterocyclyl substituted with fluoroethyl.

Embodiment 32: The compound of any one of embodiments 1-26, wherein R² is unsubstituted —NHC(═O)-3-6 membered heterocyclyl.

Embodiment 33: The compound of any one of embodiments 28-32, wherein the R² —NHC(═O)-3-6 membered heterocyclyl is —NHC(═O)-piperidinyl.

Embodiment 34: The compound of any one of embodiments 1-26, wherein R² joins with the bond denoted with *.

Embodiment 35: The compound of any one of embodiments 1-33, wherein Ring A is attached to the position of Ring B ortho to X³ and X⁴.

Embodiment 36: The compound of any one of embodiments 1-33, wherein Ring A is attached to the position of Ring B ortho to R² and meta to X³ and X⁴.

Embodiment 37: The compound of any one of embodiments 1-36, wherein X¹ is C.

Embodiment 38: The compound of any one of embodiments 1-36, wherein X¹ is N.

Embodiment 39: The compound of any one of embodiments 1-38, wherein X² is CH.

Embodiment 40: The compound of any one of embodiments 1-38, wherein X² is O.

Embodiment 41: The compound of any one of embodiments 1-38, wherein X² is S.

Embodiment 42: The compound of any one of embodiments 1-41, wherein X³ is CH.

Embodiment 43: The compound of embodiment 42, wherein the X³ CH is substituted with Ring A.

Embodiment 44: The compound of any one of embodiments 1-41, wherein X³ is N.

Embodiment 45: The compound of any one of embodiments 1-44, wherein X⁴ is C.

Embodiment 46: The compound of any one of embodiments 1-44, wherein X⁴ is N.

Embodiment 47: The compound of any one of embodiments 1-46, wherein X⁵ is CH.

Embodiment 48: The compound of embodiment 47, wherein the X⁵ CH is substituted with Ring A.

Embodiment 49: The compound of any one of embodiments 1-46, wherein X⁵ is C(═O).

Embodiment 50: The compound of any one of embodiments 1-46, wherein X⁵ is O.

Embodiment 51: The compound of any one of embodiments 1-46, wherein X⁵ is S.

Embodiment 52: The compound of any one of embodiments 1-46, wherein X⁵ is NH.

Embodiment 53: The compound of any one of embodiments 1-46, wherein X⁵ is NR^{A}.

Embodiment 54: The compound of any one of embodiments 1-46 and 53, wherein R^{A} is -(3-6 membered heterocyclyl)ₘ-(C1-C6 alkyl)ₙ.

Embodiment 55: The compound of any one of embodiments 1-46 and 53, wherein R^{A} is -(3-6 membered heterocyclyl)ₘ-(methyl)ₙ.

Embodiment 56: The compound of any one of embodiments 1-46 and 53, wherein R^{A} is -(5-6 membered heterocyclyl)ₘ-(methyl)ₙ.

Embodiment 57: The compound of any one of embodiments 1-46 and 53, wherein R^{A} is -(piperidinyl)ₘ-(methyl)ₙ.

Embodiment 58: The compound of any one of embodiments 1-46 and 53-57, wherein m is 0.

Embodiment 59: The compound of any one of embodiments 1-46 and 53-57, wherein m is 1.

Embodiment 60: The compound of any one of embodiments 1-46 and 53-59, wherein n is 0.

Embodiment 61: The compound of any one of embodiments 1-46 and 53-59, wherein n is 1.

Embodiment 62: The compound of any one of embodiments 1-46 and 53-57, wherein n is 2 and m is 1.

Embodiment 63: The compound of any one of embodiments 1-62, wherein X⁶ is CH.

Embodiment 64: The compound of embodiment 63, wherein the X⁶ CH is substituted with Ring A.

Embodiment 65: The compound of any one of embodiments 1-62, wherein X⁶ is N.

Embodiment 66: The compound of any one of embodiments 1-62, wherein X⁶ is O.

Embodiment 67: The compound of any one of embodiments 1-66, wherein X⁷ is CH.

Embodiment 68: The compound of embodiment 67, wherein the X⁷ CH is substituted with Ring A.

Embodiment 69: The compound of any one of embodiments 1-66, wherein X⁷ is CR⁷.

Embodiment 70: The compound of any one of embodiments 1-66 and 69, wherein R⁷ is C1-C6 alkyl optionally substituted with a 3-6 membered heterocyclyl optionally substituted with C(═O)OH.

Embodiment 71: The compound of any one of embodiments 1-66 and 69, wherein R⁷ is C1-C6 alkyl optionally substituted with a 3-6 membered heterocyclyl substituted with C(═O)OH.

Embodiment 72: The compound of any one of embodiments 1-66 and 69, wherein R⁷ is C1-C6 alkyl substituted with a 3-6 membered heterocyclyl optionally substituted with C(═O)OH.

Embodiment 73: The compound of any one of embodiments 1-66 and 69, wherein R⁷ is C1-C6 alkyl substituted with a 3-6 membered heterocyclyl substituted with C(═O)OH.

Embodiment 74: The compound of any one of embodiments 1-66 and 69, wherein R⁷ is C1-C6 alkyl substituted with an unsubstituted 3-6 membered heterocyclyl.

Embodiment 75: The compound of any one of embodiments 70-74, wherein the R⁷ C1-C6 membered heterocyclyl is 5-6 membered heterocyclyl.

Embodiment 76: The compound of any one of embodiments 70-74, wherein the R⁷ C1-C6 membered heterocyclyl is pyrrolidinyl.

Embodiment 77: The compound of any one of embodiments 1-66 and 69, wherein R⁷ is unsubstituted C1-C6 alkyl.

Embodiment 78: The compound of any one of embodiments 70-77, wherein the R⁷ C1-C6 alkyl is methyl.

Embodiment 79: The compound of any one of embodiments 1-66, wherein X⁷ is CH₂.

Embodiment 80: The compound of any one of embodiments 1-66, wherein X⁷ is CR^{B}R^{C}.

Embodiment 81: The compound of any one of embodiments 1-66 and 80, wherein R^{B} and R^{C} are hydrogen.

Embodiment 82: The compound of any one of embodiments 1-66 and 80, wherein R^{B} is C1-C6 alkyl and R^{C} is hydrogen.

Embodiment 83: The compound of any one of embodiments 1-66 and 80, wherein R^{B} is methyl and R^{C} is hydrogen.

Embodiment 84: The compound of any one of embodiments 1-66 and 80, wherein R^{B} is hydrogen and R^{C} is C1-C6 alkyl.

Embodiment 85: The compound of any one of embodiments 1-66 and 80, wherein R^{B} is hydrogen and R^{C} is methyl.

Embodiment 86: The compound of any one of embodiments 1-66 and 80, wherein R^{B} and R^{C} are independently selected C1-C6 alkyl.

Embodiment 87: The compound of any one of embodiments 1-66 and 80, wherein R^{B} and R^{C} are methyl.

Embodiment 88: The compound of any one of embodiments 1-66, wherein X⁷ is N.

Embodiment 89: The compound of any one of embodiments 1-36 and 42-88, wherein Ring A is

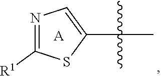

wherein the bond crossed by the wavy line is connected to Ring B or Ring C.

Embodiment 90: The compound of any one of embodiments 1-36 and 42-88, wherein Ring A is

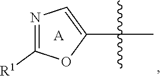

wherein the bond crossed by the wavy line is connected to Ring B or Ring C.

Embodiment 91: The compound of any one of embodiments 1-36 and 42-88, wherein Ring A is

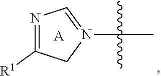

wherein the bond crossed by the wavy line is connected to Ring B or Ring C.

Embodiment 92: The compound of any one of embodiments 1-26 and 37-41, wherein Rings B and C are collectively

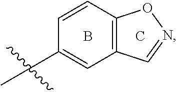

wherein the bond crossed by the wavy line is connected to Ring A.

Embodiment 93: The compound of any one of embodiments 1-26, 37-41, and 70-78, wherein Rings B and C are collectively

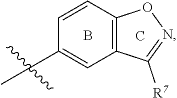

wherein the bond crossed by the wavy line is connected to Ring A.

Embodiment 94: The compound of any one of embodiments 1-26 and 37-41, wherein Rings B and C are collectively

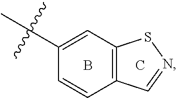

wherein the bond crossed by the wavy line is connected to Ring A.

Embodiment 95: The compound of any one of embodiments 1-26 and 37-41, wherein Rings B and C are collectively

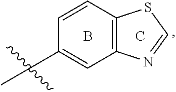

wherein the bond crossed by the wavy line is connected to Ring A.

Embodiment 96: The compound of any one of embodiments 1-26 and 37-41, wherein Rings B and C are collectively

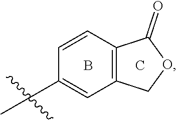

wherein the bond crossed by the wavy line is connected to Ring A.

Embodiment 97: The compound of any one of embodiments 1-26, 37-41, and 81-87, wherein Rings B and C are collectively

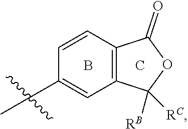

wherein the bond crossed by the wavy line is connected to Ring A.

Embodiment 98: The compound of any one of embodiments 1-26, 37-41, and 54-57, wherein Rings B and C are collectively

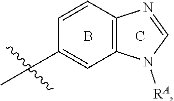

wherein the bond crossed by the wavy line is connected to Ring A.

Embodiment 99: The compound of any one of embodiments 1-26, 37-41, and 54-57, wherein Rings B and C are collectively

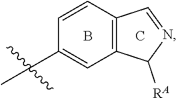

wherein the bond crossed by the wavy line is connected to Ring A.

Embodiment 100: The compound of any one of embodiments 1-34 and 37-41, wherein Rings B and C are collectively

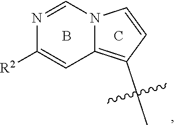

wherein the bond crossed by the wavy line is connected to Ring A.

Embodiment 101: The compound of embodiment 1, wherein the compound of Formula (III) is a compound of Formula (III-A):

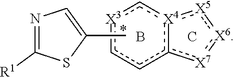

Embodiment 102: The compound of embodiment 1, wherein the compound of Formula (III) is a compound of Formula (III-B):

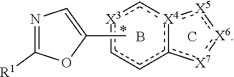

Embodiment 103: The compound of embodiment 1, wherein the compound of Formula (III) is a compound of Formula (III-C):

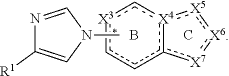

Embodiment 104: The compound of embodiment 1, wherein the compound of Formula (III) is a compound of Formula (I-IID):

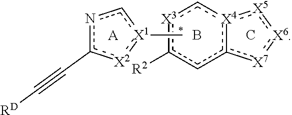

Embodiment 105: The compound of embodiment 1, wherein the compound of Formula (III) is a compound of Formula (III-E):

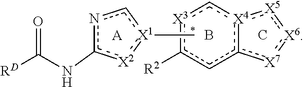

Embodiment 106: The compound of embodiment 1, wherein the compound of Formula (III) is a compound of Formula (III-F):

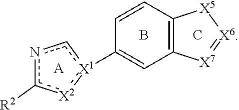

Embodiment 107: The compound of any one of embodiments 101-103 and 106, wherein R¹ is hydrogen.

Embodiment 108: The compound of any one of embodiments 101-103 and 106, wherein R¹ is —XR^{D}.

Embodiment 109: The compound of any one of embodiments 101-103 and 106, wherein X is ethynylene.

Embodiment 110: The compound of any one of embodiments 101-103 and 106, wherein X is —NHC(═O)—.

Embodiment 111: The compound of any one of embodiments 101-103 and 106, wherein X is —NHC(═O)OCH₂—.

Embodiment 112: The compound of any one of embodiments 101-111, wherein R^{D} is 3-6 membered heterocyclyl optionally substituted with C1-C6 alkyl.

Embodiment 113: The compound of any one of embodiments 101-111, wherein R^{D} is 3-6 membered heterocyclyl optionally substituted with methyl.

Embodiment 114: The compound of any one of embodiments 101-111, wherein R^{D} is 3-6 membered heterocyclyl substituted with C1-C6 alkyl.

Embodiment 115: The compound of any one of embodiments 101-111, wherein R^{D} is 3-6 membered heterocyclyl substituted with methyl.

Embodiment 116: The compound of any one of embodiments 101-111, wherein R^{D} is unsubstituted 3-6 membered heterocyclyl.

Embodiment 117: The compound of any one of embodiments 112-116, wherein the R^{D}3-6 membered heterocyclyl is 5-6 membered heterocyclyl.

Embodiment 118: The compound of any one of embodiments 112-116, wherein the R^{D}3-6 membered heterocyclyl is piperidinyl.

Embodiment 119: The compound of any one of embodiments 101-111, wherein R^{D} is 5-6 membered heteroaryl optionally substituted with C1-C6 alkyl.

Embodiment 120: The compound of any one of embodiments 101-111, wherein R^{D} is 5-6 membered heteroaryl optionally substituted with methyl.

Embodiment 121: The compound of any one of embodiments 101-111, wherein R^{D} is 5-6 membered heteroaryl substituted with C1-C6 alkyl.

Embodiment 122: The compound of any one of embodiments 101-111, wherein R^{D} is 5-6 membered heteroaryl substituted with methyl.

Embodiment 123: The compound of any one of embodiments 101-111, wherein R^{D} is unsubstituted 5-6 membered heteroaryl.

Embodiment 124: The compound of any one of embodiments 119-123, wherein the R^{D}5-6 membered heteroaryl is 6 membered heteroaryl.

Embodiment 125: The compound of any one of embodiments 119-123, wherein the R^{D}5-6 membered heteroaryl is pyridonyl.

Embodiment 126: The compound of any one of embodiments 101-111, wherein R^{D} is C6-C10 aryl optionally substituted with C1-C6 alkyl.

Embodiment 127: The compound of any one of embodiments 101-111, wherein R^{D} is C6-C10 aryl optionally substituted with methyl.

Embodiment 128: The compound of any one of embodiments 101-111, wherein R^{D} is C6-C10 aryl substituted with C1-C6 alkyl.

Embodiment 129: The compound of any one of embodiments 101-111, wherein R^{D} is C6-C10 aryl substituted with methyl.

Embodiment 130: The compound of any one of embodiments 101-111, wherein R^{D} is unsubstituted C6-C10 aryl.

Embodiment 131: The compound of any one of embodiments 126-130, wherein the R^{D}C6-C10 aryl is phenyl.

Embodiment 132: The compound of any one of embodiments 101-105 and 107-131, wherein R² is hydrogen.

Embodiment 133: The compound of any one of embodiments 101-105 and 107-131, wherein R² is —NHC(═O)-3-6 membered heterocyclyl optionally substituted with C1-C6 haloalkyl.

Embodiment 134: The compound of any one of embodiments 101-105 and 107-131, wherein R² is —NHC(═O)-3-6 membered heterocyclyl optionally substituted with fluoroethyl.

Embodiment 135: The compound of any one of embodiments 101-105 and 107-131, wherein R² is —NHC(═O)-3-6 membered heterocyclyl substituted with C1-C6 haloalkyl.

Embodiment 136: The compound of any one of embodiments 101-105 and 107-131, wherein R² is —NHC(═O)-3-6 membered heterocyclyl substituted with fluoroethyl.

Embodiment 137: The compound of any one of embodiments 101-105 and 107-131, wherein R² is unsubstituted —NHC(═O)-3-6 membered heterocyclyl.

Embodiment 138: The compound of any one of embodiments 133-137, wherein the R² —NHC(═O)-3-6 membered heterocyclyl is —NHC(═O)-piperidinyl.

Embodiment 139: The compound of any one of embodiments 101-105 and 107-131, wherein R² joins with the bond denoted with *.

Embodiment 140: The compound of any one of embodiments 101-105 and 107-138, wherein Ring A is attached to the position of Ring B ortho to X³ and X⁴.

Embodiment 141: The compound of any one of embodiments 101-105 and 107-138, wherein Ring A is attached to the position of Ring B ortho to R² and meta to X³ and X⁴.

Embodiment 142: The compound of any one of embodiments 104-106 and 107-141, wherein X¹ is C.

Embodiment 143: The compound of any one of embodiments 104-106 and 107-141, wherein X¹ is N.

Embodiment 144: The compound of any one of embodiments 104-106 and 107-143, wherein X² is CH.

Embodiment 145; The compound of any one of embodiments 104-106 and 107-143, wherein X² is O.

Embodiment 146: The compound of any one of embodiments 104-106 and 107-143, wherein X² is S.

Embodiment 147: The compound of any one of embodiments 101-105 and 107-146, wherein X³ is CH.

Embodiment 148: The compound of embodiment 147, wherein the X³ CH is substituted with Ring A.

Embodiment 149: The compound of any one of embodiments 101-105 and 107-146, wherein X³ is N.

Embodiment 150: The compound of any one of embodiments 101-105 and 107-149, wherein X⁴ is C.

Embodiment 151: The compound of any one of embodiments 101-105 and 107-149, wherein X⁴ is N.

Embodiment 152: The compound of any one of embodiments 101-151, wherein X⁵ is CH.

Embodiment 153: The compound of embodiment 152, wherein the X⁵ CH is substituted with Ring A.

Embodiment 154: The compound of any one of embodiments 101-151, wherein X⁵ is C(═O).

Embodiment 155: The compound of any one of embodiments 101-151, wherein X⁵ is 0.

Embodiment 156: The compound of any one of embodiments 101-151, wherein X⁵ is S.

Embodiment 157: The compound of any one of embodiments 101-151, wherein X⁵ is NH.

Embodiment 158: The compound of any one of embodiments 101-151, wherein X⁵ is NR^{A}.

Embodiment 159: The compound of any one of embodiments 101-151 and 158, wherein R^{A} is -(3-6 membered heterocyclyl)ₘ-(C1-C6 alkyl)ₙ.

Embodiment 160: The compound of any one of embodiments 101-151 and 158, wherein R^{A} is -(3-6 membered heterocyclyl)ₘ-(methyl)ₙ.

Embodiment 161: The compound of any one of embodiments 101-151 and 158, wherein R^{A} is -(5-6 membered heterocyclyl)ₘ-(methyl)ₙ.

Embodiment 162: The compound of any one of embodiments 101-151 and 158, wherein R^{A} is -(piperidinyl)ₘ-(methyl)ₙ.

Embodiment 163: The compound of any one of embodiments 101-151 and 158-162, wherein m is 0.

Embodiment 164: The compound of any one of embodiments 101-151 and 158-162, wherein m is 1.

Embodiment 165: The compound of any one of embodiments 101-151 and 158-164, wherein n is 0.

Embodiment 166: The compound of any one of embodiments 101-151 and 158-164, wherein n is 1.

Embodiment 167: The compound of any one of embodiments 101-151 and 158-162, wherein n is 2 and m is 1.

Embodiment 168: The compound of any one of embodiments 101-167, wherein X⁶ is CH.

Embodiment 169: The compound of embodiment 168, wherein the X⁶ CH is substituted with Ring A.

Embodiment 170: The compound of any one of embodiments 101-167, wherein X⁶ is N.

Embodiment 171: The compound of any one of embodiments 101-167, wherein X⁶ is 0.

Embodiment 172: The compound of any one of embodiments 101-171, wherein X⁷ is CH.

Embodiment 173: The compound of embodiment 172, wherein the X⁷ CH is substituted with Ring A.

Embodiment 174: The compound of any one of embodiments 101-171, wherein X⁷ is CR⁷.

Embodiment 175: The compound of any one of embodiments 101-171 and 174, wherein R⁷ is C1-C6 alkyl optionally substituted with a 3-6 membered heterocyclyl optionally substituted with C(═O)OH.

Embodiment 176: The compound of any one of embodiments 101-171 and 174, wherein R⁷ is C1-C6 alkyl optionally substituted with a 3-6 membered heterocyclyl substituted with C(═O)OH.

Embodiment 177: The compound of any one of embodiments 101-171 and 174, wherein R⁷ is C1-C6 alkyl substituted with a 3-6 membered heterocyclyl optionally substituted with C(═O)OH.

Embodiment 178: The compound of any one of embodiments 101-171 and 174, wherein R⁷ is C1-C6 alkyl substituted with a 3-6 membered heterocyclyl substituted with C(═O)OH.

Embodiment 179: The compound of any one of embodiments 101-171 and 174, wherein R⁷ is C1-C6 alkyl substituted with an unsubstituted 3-6 membered heterocyclyl.

Embodiment 180: The compound of any one of embodiments 175-179, wherein the R⁷ 3-6 membered heterocyclyl is 5-6 membered heterocyclyl.

Embodiment 181: The compound of any one of embodiments 175-179, wherein the R⁷ 3-6 membered heterocyclyl is pyrrolidinyl.

Embodiment 182: The compound of any one of embodiments 101-171 and 174, wherein R⁷ is unsubstituted C1-C6 alkyl.

Embodiment 183: The compound of any one of embodiments 175-182, wherein the R⁷ C1-C6 alkyl is methyl.

Embodiment 184: The compound of any one of embodiments 101-171, wherein X⁷ is CH₂.

Embodiment 185: The compound of any one of embodiments 101-171, wherein X⁷ is CR^{B}R^{C}.

Embodiment 186: The compound of any one of embodiments 101-171 and 185, wherein R^{B} and R^{C} are hydrogen.

Embodiment 187: The compound of any one of embodiments 101-171 and 185, wherein R^{B} is C1-C6 alkyl and R^{C} is hydrogen.

Embodiment 188: The compound of any one of embodiments 101-171 and 185, wherein R^{B} is methyl and R^{C} is hydrogen.

Embodiment 189: The compound of any one of embodiments 101-171 and 185, wherein R^{B} is hydrogen and R^{C} is C1-C6 alkyl.

Embodiment 190: The compound of any one of embodiments 101-171 and 185, wherein R^{B} is hydrogen and R^{C} is methyl.

Embodiment 191: The compound of any one of embodiments 101-171 and 185, wherein R^{B} and R^{C} are independently selected C1-C6 alkyl.

Embodiment 192: The compound of any one of embodiments 101-171 and 185, wherein R^{B} and R^{C} are methyl.

Embodiment 193: The compound of any one of embodiments 101-171, wherein X⁷ is N.

Embodiment 194: The compound of any one of embodiments 104-131 and 147-193, wherein Ring A is

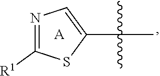

wherein the bond crossed by the wavy line is connected to Ring B or Ring C.

Embodiment 195: The compound of any one of embodiments 104-131 and 147-193, wherein Ring A is

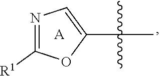

wherein the bond crossed by the wavy line is connected to Ring B or Ring C.

Embodiment 196: The compound of any one of embodiments 104-131 and 147-193, wherein Ring A is

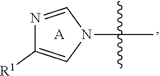

wherein the bond crossed by the wavy line is connected to Ring B or Ring C.

Embodiment 197: The compound of any one of embodiments 101-131, wherein Rings B and C are collectively

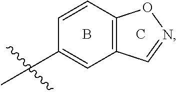

wherein the bond crossed by the wavy line is connected to Ring A.

Embodiment 198: The compound of any one of embodiments 101-131 and 175-192, wherein Rings B and C are collectively

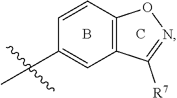

wherein the bond crossed by the wavy line is connected to Ring A.

Embodiment 199: The compound of any one of embodiments 101-105 and 107-131, wherein Rings B and C are collectively

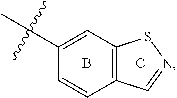

wherein the bond crossed by the wavy line is connected to Ring A.

Embodiment 200: The compound of any one of embodiments 101-131, wherein Rings B and C are collectively

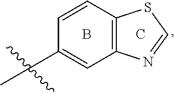

wherein the bond crossed by the wavy line is connected to Ring A.

Embodiment 201: The compound of any one of embodiments 101-131, wherein Rings B and C are collectively

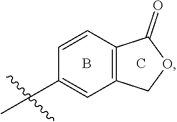

wherein the bond crossed by the wavy line is connected to Ring A.

Embodiment 202: The compound of any one of embodiments 101-131 and 186-192, wherein Rings B and C are collectively

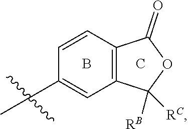

wherein the bond crossed by the wavy line is connected to Ring A.

Embodiment 203: The compound of any one of embodiments 101-105, 107-131, and 159-167, wherein Rings B and C are collectively

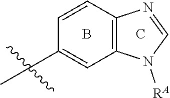

wherein the bond crossed by the wavy line is connected to Ring A.

Embodiment 204: The compound of any one of embodiments 101-105, 107-131, and 159-167, wherein Rings B and C are collectively

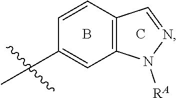

wherein the bond crossed by the wavy line is connected to Ring A.

Embodiment 205: The compound of any one of embodiments 101-105 and 107-139, wherein Rings B and C are collectively

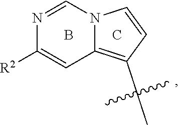

wherein the bond crossed by the wavy line is connected to Ring A.

### Exemplary Embodiments of Compounds of Formula (IV)

Embodiment 1: A compound of Formula (IV):

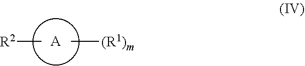

or a pharmaceutically acceptable salt thereof, wherein:
Ring A is a 12-14 membered fused tricyclic heterocyclyl comprising 2-5 nitrogen atoms or a 12-14 membered fused tricyclic heteroaryl comprising 2-5 nitrogen atoms;
R¹ is cyano, C1-C6 alkyl, —NHC(═O)(C1-C6 alkylene)ₙR^{A}, -Q-phenyl optionally substituted with 1-2 substituents independently selected from halogen, hydroxyl, and C1-C6 alkoxy; C3-C6 cycloalkyl optionally substituted with hydroxyl, —(C1-C6 alkylene)ₚ-5-10 membered heteroaryl, or 5-10 membered heterocyclyl;
R² is hydrogen, cyano, C1-C6 alkyl, —C(═O)—C1-C6 alkyl, —(SO₂)C1-C6 alkyl, —CO₂R^{B}, C1-C6 alkoxy optionally substituted with —NR^{C}R^{D};
R^{A} is 4-6 membered heterocyclyl optionally substituted with C1-C6 alkyl, or 5-10 membered heteroaryl optionally substituted with C1-C6 alkoxy or C1-C6 alkyl;
R^{B}, R^{C}, and R^{D} are independently hydrogen or C1-C6 alkyl;
Q is a bond or O;
m is 0 or 1;
n is 0 or 1; and
p is 0 or 1.

Embodiment 2: The compound of embodiment 1, wherein R¹ is cyano.

Embodiment 3: The compound of embodiment 1, wherein R¹ is C1-C6 alkyl.

Embodiment 4: The compound of embodiment 1 or 3, wherein R¹ is methyl.

Embodiment 5: The compound of embodiment 1, wherein R¹ is —NHC(═O)(C1-C6 alkylene)ₙR^{A}.

Embodiment 6: The compound of embodiment 1 or 5, wherein R¹ is —NHC(═O)(C1-C4 alkylene)ₙR^{A}.

Embodiment 7: The compound of any one of embodiment 1 or 5-6, wherein R¹ is —NHC(═O)(C1-C2 alkylene)ₙR^{A}.

Embodiment 8: The compound of any one of embodiment 1 or 5-7, wherein R^{A} is 4-6 membered heterocyclyl optionally substituted with C1-C6 alkyl.

Embodiment 9: The compound of any one of embodiment 1 or 5-8, wherein R^{A} is oxetanyl, azetidinyl, pyrrolidinyl, pyrrolidinonyl, imidazolidinyl, piperidinyl, piperazinyl, or morpholinyl; each optionally substituted with C1-C6 alkyl.

Embodiment 10: The compound of any one of embodiment 1 or 5-8, wherein R^{A} is 4-6 membered heterocyclyl substituted with C1-C6 alkyl.

Embodiment 11: The compound of any one of embodiment 1 or 5-8, wherein R^{A} is unsubstituted 4-6 membered heterocyclyl.

Embodiment 12: The compound of any one of embodiment 1 or 5-7, wherein R^{A} is 5-10 membered heteroaryl optionally substituted with C1-C6 alkoxy or C1-C6 alkyl.

Embodiment 13: The compound of any one of embodiment 1, 5-7, or 12, wherein R^{A} is 5-10 membered heteroaryl substituted with C1-C6 alkoxy.

Embodiment 14: The compound of any one of embodiment 1, 5-7, or 12-13, wherein R^{A} is 5-10 membered heteroaryl substituted with C1-C6 alkyl.

Embodiment 15: The compound of any one of embodiment 1, 5-7, or 12, wherein R^{A} is 5-6 membered heteroaryl optionally substituted with C1-C6 alkoxy or C1-C6 alkyl.

Embodiment 16: The compound of any one of embodiment 1, 5-7, 12, or 15, wherein R^{A} is pyrrolyl, imidazolyl, oxazolyl, thiazolyl, pyridinyl, pyrimidinyl, or pyrazinyl; each optionally substituted with C1-C6 alkoxy or C1-C6 alkyl.

Embodiment 17: The compound of any one of embodiment 1, 5-7, 12, or 15, wherein R^{A} is 5-6 membered heteroaryl substituted with C1-C6 alkoxy.

Embodiment 18: The compound of any one of embodiment 1, 5-7, 12, or 15, wherein R^{A} is 5-6 membered heteroaryl substituted with C1-C6 alkyl.

Embodiment 19: The compound of any one of embodiment 1, 5-7, 12, or 15, wherein R^{A} is unsubstituted 5-6 membered heteroaryl.

Embodiment 20: The compound of any one of embodiments 1 or 5-19, wherein n is 0.

Embodiment 21: The compound of any one of embodiments 1 or 5-19, wherein n is 1.

Embodiment 22: The compound of embodiment 1, wherein R¹ is -Q-phenyl optionally substituted with 1-2 substituents independently selected from halogen, hydroxyl, and C1-C6 alkoxy.

Embodiment 23: The compound of embodiment 1 or 22, wherein R¹ is -Q-phenyl substituted with one or two independently selected halogen.

Embodiment 24: The compound of any one of embodiments 1 or 22-23, wherein R¹ is -Q-phenyl substituted with one halogen.

Embodiment 25: The compound of embodiment 1 or 22, wherein R¹ is -Q-phenyl substituted with one hydroxyl.

Embodiment 26: The compound of embodiment 1 or 22, wherein R¹ is -Q-phenyl substituted with one or two independently selected C1-C6 alkoxy.

Embodiment 27: The compound of embodiment 1 or 22, wherein R¹ is -Q-phenyl substituted with one halogen and one C1-C6 alkoxy.

Embodiment 28: The compound of embodiment 1 or 22, wherein R¹ is -Q-unsubstituted phenyl.

Embodiment 29: The compound of any one of embodiments 1 or 22-28, wherein Q is a bond.

Embodiment 30: The compound of any one of embodiments 1 or 22-28, wherein Q is 0.

Embodiment 31: The compound of embodiment 1, wherein R¹ is C3-C6 cycloalkyl optionally substituted with hydroxyl.

Embodiment 32: The compound of embodiment 1 or 31, wherein R¹ is C3-C6 cycloalkyl substituted with hydroxyl.

Embodiment 33: The compound of embodiment 1 or 31, wherein R¹ is unsubstituted C3-C6 cycloalkyl.

Embodiment 34: The compound of embodiment 1, wherein R¹ is —(C1-C6 alkylene)ₚ-5-10 membered heteroaryl.

Embodiment 35: The compound of embodiment 1 or 34, wherein R¹ is —(C1-C6 alkylene)ₚ-5-6 membered heteroaryl.

Embodiment 36: The compound of any one of embodiments 1 or 34-35, wherein R¹ is —(C1-C6 alkylene)ₚ-pyrrolyl, —(C1-C6 alkylene)ₚ-imidazolyl, —(C1-C6 alkylene)ₚ-oxazolyl, —(C1-C6 alkylene)ₚ-thiazolyl, —(C1-C6 alkylene)ₚ-pyridinyl, —(C1-C6 alkylene)ₚ-pyrimidinyl, or —(C1-C6 alkylene)ₚ-pyrazinyl.

Embodiment 36: The compound of any one of embodiments 1 or 34-35, wherein R¹ is —(C1-C2 alkylene)ₚ-5-6 membered heteroaryl.

Embodiment 38: The compound of any one of embodiments 1, 34-35, or 37, wherein R¹ is —(C1-C2 alkylene)ₚ-pyrrolyl, —(C1-C2 alkylene)ₚ-imidazolyl, —(C1-C2 alkylene)ₚ-oxazolyl, —(C1-C2 alkylene)ₚ-thiazolyl, —(C1-C2 alkylene)ₚ-pyridinyl, —(C1-C2 alkylene)ₚ-pyrimidinyl, or —(C1-C2 alkylene)ₚ-pyrazinyl.

Embodiment 39: The compound of any one of embodiments 1 or 34-39, wherein p is 0.

Embodiment 40: The compound of any one of embodiments 1 or 34-39, wherein p is 1.

Embodiment 41: The compound of embodiment 1, wherein R¹ is a 5-10 membered heterocyclyl.

Embodiment 42: The compound of embodiment 1 or 41, wherein R¹ is a 5-6 membered heterocyclyl.

Embodiment 43: The compound of any one of embodiments 1 or 41-42, wherein R¹ is tetrahydropyran or dihydropyran.

Embodiment 44: The compound of embodiment 1 or 41, wherein R¹ is a fused bicyclic 9-10 membered heterocyclyl.

Embodiment 45: The compound of any one of embodiments 1, 41, or 44, wherein R¹ is [1,3]dioxolo[4,5-b]pyridine, benzo[d][1,3]dioxole, 2,3-dihydrofuro[2,3-b]pyridine, 2,3-dihydrobenzofuran, or 2,3-dihydrofuro[3,2-b]pyridine.

Embodiment 46: The compound of any one of embodiments 1-45, wherein R² is hydrogen.

Embodiment 47: The compound of any one of embodiments 1-45, wherein R² is cyano.

Embodiment 48: The compound of any one of embodiments 1-45, wherein R² is C1-C6 alkyl.

Embodiment 49: The compound of any one of embodiments 1-45 or 48, wherein R² is methyl.

Embodiment 50: The compound of any one of embodiments 1-45, wherein R² is —C(═O)—C1-C6 alkyl.

Embodiment 51: The compound of any one of embodiments 1-45 or 50, wherein R² is —C(═O)CH₃.

Embodiment 51: The compound of any one of embodiments 1-45, wherein R² is —(SO₂)C1-C6 alkyl.

Embodiment 53: The compound of any one of embodiments 1-45 or 52, wherein R² is —(SO₂)CH₃.

Embodiment 54: The compound of any one of embodiments 1-45, wherein R² is —CO₂R^{B}.

Embodiment 55: The compound of any one of embodiments 1-45 or 54, wherein R^{B} is C1-C6 alkyl.

Embodiment 56: The compound of any one of embodiments 1-45 or 54-55, wherein R² is —CO₂CH₃.

Embodiment 57: The compound of any one of embodiments 1-45 or 54, wherein R^{B} is hydrogen.

Embodiment 58: The compound of any one of embodiments 1-45, wherein R² is C1-C6 alkoxy optionally substituted with —NR^{C}R^{D}.

Embodiment 59: The compound of any one of embodiments 1-45 or 58, wherein R² is C1-C6 alkoxy substituted with —NR^{C}R^{D}.

Embodiment 60: The compound of any one of embodiments 1-45 or 58-59, wherein R² is ethoxy substituted with —NR^{C}R^{D}.

Embodiment 61: The compound of any one of embodiments 1-45 or 58-60, wherein R^{C} is C1-C6 alkyl.

Embodiment 62: The compound of any one of embodiments 1-45 or 58-61, wherein R^{C} is methyl.

Embodiment 63: The compound of any one of embodiments 1-45 or 58-60, wherein R^{C} is hydrogen, Embodiment 64: The compound of any one of embodiments 1-45 or 58-63, wherein R^{D} is C1-C6 alkyl.

Embodiment 65: The compound of any one of embodiments 1-45 or 58-64, wherein R^{D} is methyl.

Embodiment 66: The compound of any one of embodiments 1-45 or 58-63, wherein R^{D} is hydrogen, Embodiment 67: The compound of any one of embodiments 1-45 or 58-60, wherein R^{C} and R^{D} are the same.

Embodiment 68: The compound of any one of embodiments 1-45 or 58-60, wherein R^{C} and R^{D} are different.

Embodiment 69: The compound of any one of embodiments 1-45 or 58-60, wherein R^{C} and R^{D} are each hydrogen.

Embodiment 70: The compound of any one of embodiments 1-45 or 58-60, wherein R^{C} and R^{D} are each methyl.

Embodiment 71: The compound of any one of embodiments 1-45 or 58-60, wherein one of R^{C} and R^{D} is hydrogen and the other of R^{C} and R^{D} is C1-C6 alkyl.

Embodiment 72: The compound of any one of embodiments 1-45 or 58, wherein R² is unsubstituted C1-C6 alkoxy.

Embodiment 73: The compound of any one of embodiments 1-45, 58, or 72, wherein R² is methoxy.

Embodiment 74: The compound of any one of embodiments 1-73, wherein m is 1.

Embodiment 75: The compound of any one of embodiments 1-73, wherein m is 0.

Embodiment 76: The compound of any one of embodiments 1-75, wherein Ring A is 12-14 membered fused tricyclic heterocyclyl comprising 2-4 nitrogen atoms.

Embodiment 77: The compound of any one of embodiments 1-76, wherein Ring A is 12-14 membered fused tricyclic heterocyclyl comprising 2-3 nitrogen atoms.

Embodiment 78: The compound of any one of embodiments 1-77, wherein Ring A is 12-14 membered fused tricyclic heterocyclyl comprising 2 nitrogen atoms.

Embodiment 79: The compound of any one of embodiments 1-78, wherein Ring A is 12-14 membered fused tricyclic heterocyclyl comprising one all-carbon 5-6 membered ring.

Embodiment 80: The compound of any one of embodiments 1-79, wherein Ring A is 12-14 membered fused tricyclic heterocyclyl further comprising one oxygen atom.

Embodiment 81: The compound of any one of embodiments 1-75, wherein Ring A is 12-14 membered fused tricyclic heteroaryl comprising 2-4 nitrogen atoms.

Embodiment 82: The compound of any one of embodiments 1-75 or 81, wherein Ring A is 12-14 membered fused tricyclic heteroaryl comprising 2-3 nitrogen atoms.

Embodiment 83: The compound of any one of embodiments 1-75 or 81-82, wherein Ring A is 12-14 membered fused tricyclic heteroaryl comprising 2 nitrogen atoms.

Embodiment 84: The compound of any one of embodiments 1-75 or 81-83, wherein Ring A is 12-14 membered fused tricyclic heteroaryl comprising one all-carbon 6 membered ring.

Embodiment 85: The compound of any one of embodiments 1-75, wherein Ring A is 6H-isochromeno[3,4-d]pyrimidine, 5,7-dihydro-2H-imidazo[4′,5′:4,5]benzo[1,2-d]oxazole-2,6(3H)-dione, 5,7-dihydroimidazo[4,5-f]indazol-6(1H)-one, oxazolo[4,5-g]isoquinolin-2(1H)-one; 1,7-dihydro-6H-oxazolo[5,4-f]indazol-6-one, 6,7,8,9-tetrahydro-3H-pyrrolo[2,3-c][2,7]naphthyridine, benzo[c][2,6]naphthyridine, 1,3,4,5-tetrahydrobenzo[c][1,7]naphthyridin-6(2H)-one, 3H-pyrazolo[3,4-c]quinolone, 2,3,4,7-tetrahydro-1H-pyrrolo[2,3-c][2,6]naphthyridine, or pyrrolo[1,2-a]quinoxalin-4(5H)-one.

Embodiment 86: The compound of any one of embodiments 1-75, wherein Ring A is

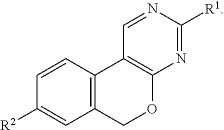

Embodiment 87: The compound of any one of embodiments 1-75, wherein Ring A is

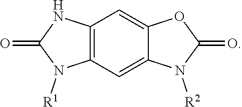

Embodiment 88: The compound of any one of embodiments 1-75, wherein Ring A is

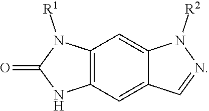

Embodiment 89: The compound of any one of embodiments 1-75, wherein Ring A is

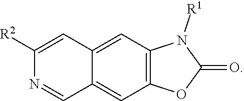

Embodiment 90: The compound of any one of embodiments 1-75, wherein Ring A is

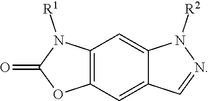

Embodiment 91: The compound of any one of embodiments 1-75, wherein Ring A is

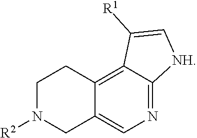

Embodiment 92: The compound of any one of embodiments 1-75, wherein Ring A is

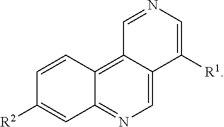

Embodiment 93: The compound of any one of embodiments 1-75, wherein Ring A is

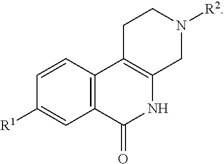

Embodiment 94: The compound of any one of embodiments 1-75, wherein Ring A is

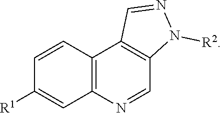

Embodiment 95: The compound of any one of embodiments 1-75, wherein Ring A is

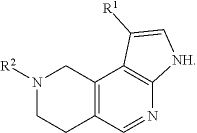

Embodiment 96: The compound of any one of embodiments 1-75, wherein Ring A is

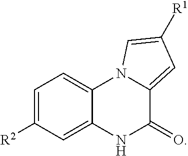

Further exemplary Embodiments of compounds of Formulae (I), (II), (III), and (IV):

Embodiment 1: A compound selected from a compound in Table 1, Table 2, Table 3, or Table 4, or a pharmaceutically acceptable salt of any of the foregoing; or a compound described in the Examples section herein, or a pharmaceutically acceptable salt thereof.

Embodiment 2: A pharmaceutical composition comprising a compound of Embodiment 1 or any one of the Embodiments of compounds of Formulae (I), (II), (III), and (IV), or a pharmaceutically acceptable salt thereof, and pharmaceutically acceptable diluent or carrier.

Embodiment 3: A method for treating a neurological disorder in a subject in need thereof, the method comprising administering to the subject a therapeutically effective amount of a compound of Embodiment 1 or any one of the Embodiments of compounds of Formulae (I), (II), (III), and (IV), or a pharmaceutically acceptable salt thereof, or a pharmaceutical composition according to Embodiment 2.

Embodiment 4: The method of Embodiment 3, wherein the neurological disorder is selected from the group consisting of Down Syndrome, Alzheimer's disease, and Alzheimer's disease associated with Down Syndrome.

Embodiment 5: The method of Embodiment 3 or 4, wherein the neurological disorder is selected Alzheimer's disease associated with Down syndrome.

Embodiment 6: A method of treating a subject, the method comprising administering a therapeutically effective amount of a compound of Embodiment 1 or any one of the Embodiments of compounds of Formulae (I), (II), (III), and (IV), or a pharmaceutically acceptable salt thereof, or a pharmaceutical composition according to Embodiment 2, to a subject having a clinical record that indicates that the subject has a dysregulation of a DYRK1A gene, DYRK1A protein, or expression or activity or level of any of the same.

Embodiment 7: A method of treating a DYRK1A-associated neurological disorder in a subject, the method comprising administering to a subject identified or diagnosed as having a DYRK1A-associated neurological disorder a therapeutically effective amount of a compound of Embodiment 1 or any one of the Embodiments of compounds of Formulae (I), (II), (III), and (IV), or a pharmaceutically acceptable salt thereof, or a pharmaceutical composition according to Embodiment 2.

Embodiment 8: A method of treating a DYRK1A-associated neurological disorder in a subject, the method comprising:
(a) determining that the neurological disorder in the subject is a DYRK1A-associated neurological disorder; and
(b) administering to the subject a therapeutically effective amount of a compound of Embodiment 1 or any one of the Embodiments of compounds of Formulae (I), (II), (III), and (IV), or a pharmaceutically acceptable salt thereof, or a pharmaceutical composition according to Embodiment 2.

Embodiment 9: A method for treating a neurological disorder in a subject in need thereof, the method comprising (a) determining that the neurological disorder is associated with a dysregulation of a DYRK1A gene, a DYRK1A protein, or expression or activity or level of any of the same; and (b) administering to the subject a therapeutically effective amount of a compound of Embodiment 1 or any one of the Embodiments of compounds of Formulae (I), (II), (III), and (IV), or a pharmaceutically acceptable salt thereof, or a pharmaceutical composition according to Embodiment 2.

Embodiment 10: The method of Embodiment 8 or 9, wherein the step of determining that the neurological disorder in the subject is a DYRK1A-associated neurological disorder includes performing an assay to detect dysregulation in a DYRK1A gene, a DYRK1A protein, or expression or activity or level of any of the same in a sample from the subject.

Embodiment 11: A method for treating a neurological disorder in a subject in need thereof, the method comprising (a) determining that the neurological disorder is associated with Down Syndrome; and (b) administering to the subject a therapeutically effective amount of a compound of Embodiment 1 or any one of the Embodiments of compounds of Formulae (I), (II), (III), and (IV), or a pharmaceutically acceptable salt thereof, or a pharmaceutical composition according to Embodiment 2.

Embodiment 12: The method of Embodiment 11, wherein the step of determining that the neurological disorder in the subject is associated with Down Syndrome includes performing an assay on a sample from the subject.

Embodiment 13: The method of any one of Embodiments 8-11, wherein the assay is selected from the group consisting of sequencing, immunohistochemistry, enzyme-linked immunosorbent assay, and fluorescence in situ hybridization (FISH).

Embodiment 14: The method of any one of embodiments 7-10, wherein the DYRK1A-associated neurological disorder is selected from the group consisting of Down Syndrome, Alzheimer's disease, and Alzheimer's disease associated with Down Syndrome.

Embodiment 15: The method of any one of embodiments 7-10 and 14, wherein the DYRK1A-associated neurological disorder is Alzheimer's disease associated with Down syndrome.

Embodiment 16: A method for modulating DYRK1A in a mammalian cell, the method comprising contacting the mammalian cell with a therapeutically effective amount of a compound of Embodiment 1 or any one of the Embodiments of compounds of Formulae (I), (II), (III), and (IV), or a pharmaceutically acceptable salt thereof.

Embodiment 17: The method of embodiment 16, wherein the contacting occurs in vivo.

Embodiment 18: The method of embodiment 16, wherein the contacting occurs in vitro.

Embodiment 19: The method of any one of embodiments 16-18, wherein the mammalian cell is a mammalian neural cell.

Embodiment 20: The method of embodiment 19, wherein the mammalian neural cell is a mammalian DYRK1A-associated neural cell.

Embodiment 21: The method of any one of embodiments 16-20, wherein the cell has a dysregulation of a DYRK1A gene, a DYRK1A protein, or expression or activity or level of any of the same.

Embodiment 22: The method of any one of embodiments 16-21, wherein the cell has a chromosomal abnormality associated with Down Syndrome.

### Examples

### Compound Preparation

The compounds disclosed herein can be prepared in a variety of ways using commercially available starting materials, compounds known in the literature, or from readily prepared intermediates, by employing standard synthetic methods and procedures either known to those skilled in the art, or in light of the teachings herein. The synthesis of the compounds disclosed herein can be achieved by generally following the schemes provided herein, with modification for specific desired substituents.

Standard synthetic methods and procedures for the preparation of organic molecules and functional group transformations and manipulations can be obtained from the relevant scientific literature or from standard textbooks in the field. Although not limited to any one or several sources, classic texts such as R. Larock, Comprehensive Organic Transformations, VCH Publishers (1989); L. Fieser and M. Fieser, Fieser and Fieser's Reagents for Organic Synthesis, John Wiley and Sons (1994); Smith, M. B., March, J., March's Advanced Organic Chemistry: Reactions, Mechanisms, and Structure, 5th edition, John Wiley & Sons: New York, 2001; and Greene, T. W., Wuts, P. G. M., Protective Groups in Organic Synthesis, 3rd edition, John Wiley & Sons: New York, 1999, are useful and recognized reference textbooks of organic synthesis known to those in the art. The following descriptions of synthetic methods are designed to illustrate, but not to limit, general procedures for the preparation of compounds of the present disclosure.

The synthetic processes disclosed herein can tolerate a wide variety of functional groups; therefore, various substituted starting materials can be used. The processes generally provide the desired final compound at or near the end of the overall process, although it may be desirable in certain instances to further convert the compound to a pharmaceutically acceptable salt thereof.

### NMR

¹H Nuclear magnetic resonance (NMR) spectroscopy (¹H NMR) was carried out on a Bruker Avance 400 MHz.

### Liquid Chromatography-Mass Spectrometry

Unless otherwise indicated, Liquid Chromatography-Mass Spectrometry (LCMS) was taken on a quadruple Mass Spectrometer on Shimadzu LCMS 2010 (Column: Shim-pack XR-ODS (3.0×30 mm, 2.2 m)) operating in ESI (+) ionization mode. Flow Rate: 0.8 mL/min, Acquire Time: 2 min or 3 min, Wavelength: UV220, Oven Temp.: 50° C.

### Preparative High-Performance Liquid Chromatography

Preparative High-Performance Liquid Chromatography (Prep-HPLC) was conducted using one or more of methods A-H.

Method A (used in, e.g., Examples 1, 2, 7, 28, 42, 48, 49, 50, 51, and 53):

Column: Fuji C18 (300×25), YMC (250×20); Wavelength: 220 nm; Mobile phase: A CH₃CN (0.10% TFA); B water (0.10% TFA); Flow rate: 25 mL/min; Injection Volume: 2 mL; Run time: 20 min; Equilibration: 3 min.

Method B (used in, e.g., Examples 1, 2, 7, 16, 17, 25, 26, 28, 29, 30, 32, 34, 37, 42, and 46-53):

Column: Fuji C18 (300×25), YMC 250×20; Wavelength: 220 nm; Mobile phase: A CH₃CN (0.05% NH₃·H₂O as an additive); B water (0.05% NH₃·H₂O as an additive); Flow rate: 25 mL/min; Injection Volume: 2 mL; Run time: 20 min; Equilibration: 3 min.

Method C (used in, e.g., Examples 3, 4, 6, and 8):

Column: Phenomenex luna C18 100*40 mm*3 um, YMC (250×20); Wavelength: 220 nm; Mobile phase: A CH₃CN; B water (0.225% FA); Flow rate: 25 mL/min; Injection Volume: 1 mL; Run time: 15 min; Equilibration: 3 min.

Method D (used in, e.g., Examples 9-11, 13, 14, 20-24, 27, 31, 33, 35, 36, 38-41, and 43-45):

Column: Phenomenex luna C18 (100×25), YMC (250×20); Wavelength: 220 nm; Mobile phase: A CH₃CN; B water (0.225% FA); Flow rate: 25 mL/min; Injection Volume: 2 mL; Run time: 10 min; Equilibration: 3 min.

Method E (used in, e.g., Example 12):

Column: Fuji C18 (300×25), YMC (250×20); Wavelength: 220 nm; Mobile phase: A CH₃CN (0.225% FA); B water (0.225% FA); Flow rate: 25 mL/min; Injection Volume: 2 mL; Run time: 20 min; Equilibration: 3 min.

Method E (used in, e.g., Example 15):

Column: Phenomenex luna C18 (100×25), YMC (250×20); Wavelength: 220 nm; Mobile phase: A CH₃CN; B water (0.04% HCl); Flow rate: 25 mL/min; Injection Volume: 2 mL; Run time: 10 min; Equilibration: 3 min.

Method F (used in, e.g., Example 18):

Column: Phenomenex luna C18 (100×25), YMC (250×20); Wavelength: 220 nm; Mobile phase: A CH₃CN; B water (0.05% HCl); Flow rate: 25 mL/min; Injection Volume: 2 mL; Run time: 10 min; Equilibration: 3 min.

Method G (used in, e.g., Example 19):

Column: Phenomenex luna C18 100×40 mm×3 um, YMC (250×20); Wavelength: 220 nm; Mobile phase: A CH₃CN; B water (0.05% HCl); Flow rate: 25 mL/min; Injection Volume: 1 mL; Run time: 15 min; Equilibration: 3 min.

Method H (used in, e.g., Example 24):

Column: Phenomenex luna C18 (100×25), YMC (250×20); Wavelength: 220 nm; Mobile phase: A CH₃CN; B water (0.1% TFA); Flow rate: 25 mL/min; Injection Volume: 2 mL; Run time: 10 min; Equilibration: 3 min.

Method I: Column: Phenomenex luna C18 (100×25), YMC (250×20); Wavelength: 220 nm; Mobile phase: A CH₃CN; B water (0.025% FA); Flow rate: 25 mL/min; Injection Volume: 2 mL; Run time: 10 min; Equilibration: 3 min.

Method J: Column: Fuji C18 (300×25), YMC 250×20; Wavelength: 220 nm; Mobile phase: A CH₃CN (0.05% FA as an additive); B water (0.05% FA as an additive); Flow rate: 25 mL/min; Injection Volume: 2 mL; Run time: 20 min; Equilibration: 3 min.

### Supercritical Fluid Chromatography

Supercritical fluid chromatography (SFC) methods A and B are described as follows:

Method A (used in, e.g., Example 5, 10, 11, 14):

Column: Daicel Chiralpak AD (250×30), YMC (250×20); Wavelength: 220 nm; Mobile phase: B: 55%, EtOH (0.1% NH₃H₂O); Flow rate: 80 mL/min; Injection Volume: 100 mL.

Method B (used in, e.g., Example 38)

Column: Daicel Chiralpak IE (250 mm×30 mm, 10 um); mobile phase: [0.10% NH₃H₂O in EtOH].

### Abbreviations

| Abbreviation | Meaning |
|---|---|
| (HCHO)ₙ | paraformaldehyde |
| (t-Bu₃P)₂Pd | bis(tri-tert-butylphosphine)palladium(0) |
| (tol)₃P | tri(o-tolyl)phosphine |
| AcCl | acetyl chloride |
| ACN | acetonitrile |
| aq | aqueous |
| Bis-pin | bis(pinacolato)diboron |
| BINAP | (±)-2,2′-Bis(diphenylphosphino)-1,1′-binaphthalene |
| BnBr | (bromomethyl)benzene |
| BnCl | benzyl chloride |
| BnNHNH₂ | benzylhydrazine hydrochloride |
| BnSH | benzylthiol |
| Boc | t-butyloxy carbonyl |
| Boc₂O | di-tert-butyl dicarbonate |
| BocNH₂ | tert-butyl carbamate |
| BPO | Benzoyl Peroxide |
| BrettPhos | 2-dicyclohexylphosphino-2′,4′,6′-triisopropyl-3,6-dimethoxybiphenyl |
| BrettPhos Pd G3 | methanesulfonato(2-dicyclohexylphosphino-3,6-dimethoxy-2′,4′,6′-tri- |
| | i-propyl-1,1′-biphenyl)(2′-amino-1,1′-biphenyl-2-yl)palladium(II) |
| brs | broad singlet |
| C¹⁸ | octyldecylsilane |
| CbzCl | benzyl carbonochloridate |
| Cbz | carbobenzyloxy |
| CDCl₃ | deuterated chloroform |
| CDI | carbonyl diimidazole |
| CuCN | Copper(I) cyanide |
| Cul | Copper (I) iodide |
| CPME | Cyclopentyl methyl ether |
| CyJohnphos | (2-Biphenyl)dicyclohexylphosphine |
| CH₂Cl₂, DCM | dichloromethane |
| CH₃CN, MeCN | acetonitrile |
| ClCO₂Bn | benzyl carbonochloridate |
| CO | carbon monoxide |
| conc. | concentrated |
| Cs₂CO₃ | cesium carbonate |
| CuI | copper (I) iodide |
| d | doublet |
| DABCO | 1,4-diazabicyclo[2.2.2]octane |
| DAST | diethylaminosulfur trifluoride |
| DCE | dichloroethane |
| dd | doublet of doublets |
| DIBAL-H | diisobutylaluminum hydride |
| DIEA, DIPEA | diisopropylethylamine |
| DIAD | diisopropyl azodicarboxylate |
| DMF | N,N-dimethylformamide |
| Dioxane | 1,4-dioxane |
| DMF-DMA | N,N-dimethylformamide dimethylacetamide |
| DMP | Dess Martin periodinane |
| DMSO | dimethyl sulfoxide |
| DMSO-d₆ | deuterated dimethylsulfoxide |
| DPPA | diphenylphosphoryl azide |
| Dtbpy | 4,4-di-tert-butyl-2,2′ dipyridine |
| EDCI | 1-ethyl-3-(3-dimethylaminopropyl)carbodiimide |
| eq, equiv | equivalent(s) |
| ESI | electrospray ionization |
| EtI | Ethyl iodide |
| Et₂O | diethyl ether |
| Et₃SiH | triethylsilane |
| EtOAc | ethyl acetate |
| FA | formic acid |
| HATU | N,N,N,N-tetramethyl-o-(7-azabenzotriazol-1-yl)uronium |
| | hexafluorophosphate |
| g | gram(s) |
| Grubb′s II | Benzylidene[1,3-bis(2,4,6-trimethylphenyl)-2- |
| catalyst | imidazolidinylidene]dichloro(tricyclohexylphosphine)rutheniumum |
| H₂ | hydrogen |
| H₂O | water |
| H₂SO₄ | sulfuric acid |
| HBPin | 4,4,5,5-Tetramethyl-1,3,2-dioxaborolane |
| HBr | hydrogen bromide |
| HC(OMe)₃ | trimethoxymethane |
| HCHO | formaldehyde |
| HCl | hydrochloric acid |
| HCOOEt | ethyl formate |
| HCOONH₄ | ammonium formate |
| HF/Py | hydrogen fluoride-pyridine |
| HI | hydrogen iodide |
| HOAc, AcOH | acetic acid |
| HOBt | hydroxy benzotriazole |
| HPLC | high performance liquid chromatography |
| hr, h | hour(s) |
| I₂ | iodine |
| [Ir(OMe)(COD)]₂ | Bis(1,5-cyclooctadiene)di-μ-methoxy diiridium(I) |
| i-Pr₂NH | diisopropylamine |
| i-PrOH | isopropanol |
| K₂CO₃ | potassium carbonate |
| KHCO₃ | potassium hydrogen carbonate |
| KOAc | potassium acetate |
| KOH | potassium hydroxide |
| LCMS | liquid chromatography-mass spectrometry |
| LDA | lithium diisopropylamide |
| LiAlH₄, LAH | lithium aluminum hydride |
| LiHMDS | lithium bis(trimethylsilyl)amide |
| LiCl | lithium chloride |
| LiOH | lithium hydroxide |
| m | multiplet |
| M | molar |
| Me | methyl |
| MeCN | acetonitrile |
| Me₂NH | dimethylamine |
| Mel | iodomethane |
| MsCl | Methanesulfonyl chloride |
| Me₂SO₄ | Dimethyl sulfate |
| MeOH | methanol |
| mg | milligram(s) |
| MgSO₄ | magnesium sulfate |
| MHz | megahertz |
| min | minute(s) |
| mL | milliliter(s) |
| mm | millimeter(s) |
| mmol | millimole(s) |
| MnO₂ | manganese dioxide |
| MW | microwave |
| N₂ | diatomic nitrogen |
| NaOAc | Sodium acetate |
| Na₂CO₃ | sodium carbonate |
| NaBH(OAc)₃ | sodium triacetoxy borohydride |
| NaBH₃CN | sodium cyanoborohydride |
| NaClO | sodium hypochlorite |
| NaClO₂ | sodium chlorite |
| NaH | sodium hydride |
| NaH₂PO₄ | monosodium phosphate |
| NaHCO₃ | sodium bicarbonate |
| NaOMe, MeONa | sodium methoxide |
| NBS | N-bromosuccinimide |
| NCS | N-Chlorosuccinimide |
| n-Bu3SnH | Tributyltin hydride |
| n-Bu₃P | tri-n-butylphosphine |
| n-Bu₃SnCl | tributyltin chloride |
| n-BuLi | n-butyllithium |
| NH₂NH₂ | hydrazine |
| NH₂OH | hydroxylamine |
| NH₃ | ammonia |
| NH₄Cl | ammonium chloride |
| NIS | N-iodosuccinimide |
| NMP | N-methyl pyrrolidinone |
| NMI | 1-methylimidazole |
| NMR | nuclear magnetic resonance |
| ° C. | degrees celsius |
| PCy₃ | tricyclohexylphosphine |
| Pd₂(dba)₃-CHCl₃ | tris(dibenzylideneacetone)dipalladium(0)-chloroform adduct |
| Pd(dppf)Cl₂ | [1,1′-bis(diphenylphosphino)ferrocene]dichloropalladium(II) |
| Pd(dppf)Cl₂- | 1,1′-bis(diphenylphosphino)ferrocene-palladium(II)dichloride |
| CH₂Cl₂ | dichloromethane complex |
| Pd(dtbpf)Cl₂ | [1,1′-Bis(di-tert-butylphosphino)ferrocene]dichloropalladium(II) |
| Pd/C | Palladium (0) on activated carbon |
| Pd(OAc)₂ | palladium (II) acetate |
| PPh₃ | Triphenylphosphine |
| PPTS | Pyridinium p-toluenesulfonate |
| Pd(PPh₃)₄ | Tetrakis(triphenylphospine)palladium (0) |
| Pd(PPh₃)Cl₂ | bis(triphenylphosphine)palladium(II) dichloride |
| Pd/C | palladium on carbon |
| PE | petroleum ether |
| PhI(OAc)₂ | phenyliodonium acetate |
| PhNTf₂ | N-phenyl-bis(trifluoromethanesulfonimide) |
| PivOH | pivalic acid |
| PPA | Polyphosphoric acid |
| POCl₃ | phosphorus oxychloride |
| prep-HPLC | preparative high-performance liquid chromatography |
| prep-TLC | preparative thin layer chromatography |
| psi | pounds per square inch |
| PtO₂ | platinum dioxide |
| RT | room temperature |
| Ruphos-Pd-G2 | Chloro(2-dicyclohexylphosphino-2′,6′-diisopropoxy-1,1′-biphenyl)[2- |
| | (2′-amino-1,1′-biphenyl)]palladium(II) |
| Ruphos | 2-Dicyclohexylphosphino-2′,6′-diisopropoxy biphenyl |
| s | singlet |
| Sat. | saturated |
| SEMCl | 2-(trimethylsily)ethoxymethyl chloride |
| SFC | supercritical fluid chromatography |
| SiO₂ | silica gel |
| SO₂Cl₂ | sulfonyl chloride |
| Sodium | sodium phosphate |
| phosphate buffer | |
| Speedvac | Savant SC250EXP SpeedVac Concentrator |
| Sulfolane | 126-Thiolane-1,1-dione |
| T3P | propylphosphonic anhydride |
| TABF | tetrabutylammonium fluoride trihydrate |
| TBAB | Tetrabutylammonium bromide |
| TBAF | tetrabutylammonium fluoride |
| TCFH | N,N,N,N-tetramethylchloroformamidinium hexafluorophosphate |
| TBSCl | tert-butyldimethylsilyl chloride |
| t-BuOH | tert-butanol |
| t-BuOK | potassium tert-butoxide |
| t-BuONO | tert-Butyl nitrite |
| t-Bu₃PHBF₄ | Tri-tert-butylphosphonium tetrafluoroborate |
| TEA, Et₃N | triethylamine |
| TEMPO | 2,2,6,6-tetramethylpiperidinooxy |
| TFA | trifluoroacetic acid |
| TFAA | trifluoroacetic anhydride |
| TfOH | trifluoromethanesulfonic acid |
| THF | tetrahydrofuran |
| TLC | thin layer chromatography |
| TMAD | Tetramethylazodicarboxamide |
| TMSCHNH₂ | Trimethylsilyldiazomethane |
| TMSCN | Trimethylsilyl cyanide |
| TMSCl | trimethylsilyl chloride |
| TosMIC | toluenesulfonylmethyl isocyanide |
| Troc-Cl | 2,2,2-trichloroethoxycarbonyl chloride |
| Trt | Trityl protecting group |
| Trt-Cl | trityl chloride |
| TsCl | para-toluenesulfonyl chloride |
| um, mm | micrometer(s) |
| UV | ultraviolet |
| XantPhos | (9,9-dimethyl-9H-xanthene-4,5-diyl)bis(diphenylphosphane) |
| XantPhos Pd G2 | chloro[(4,5-bis(diphenylphosphino)-9,9-dimethylxanthene)-2-(2- |
| | amino-1,1-biphenyl)]palladium(II) |
| Xphos | 2-dicyclohexylphosphino-2′,4′,6′-triisopropylbiphenyl |
| Xphos-Pd-G3 | (2-dicyclohexylphosphino-2′,4′,6′-triisopropyl-1,1′-biphenyl)[2-(2′- |
| | amino-1,1′-biphenyl)]palladium(II) methanesulfonate |
| X3 | Three times |
| X2 | Two times |
| ZnF₂ | zinc (II) fluoride |

### Synthesis

The synthesis of the reported compounds was performed according to the following procedures.

### Preparation of Intermediates of Formula (I)

### Preparation of 6-bromo-4-iodoisoquinolin-3-amine (Int-I)

To a mixture of compound 6-bromoisoquinolin-3-amine (1.00 g, 4.48 mmol) in DMF (15 mL) was added NIS (1.01 g, 4.48 mmol) at 0° C. The reaction mixture was stirred at 0° C. for 1 hour to give a yellow mixture. The reaction mixture was concentrated to dryness in vacuum. The residue was purified by flash column (SiO₂, EtOAc 0% to 30% in petroleum ether (PE)) to give Int-I (1.20 g, yield: 77%) as a yellow solid. ¹H NMR (400 MHz, CDCl₃) δ 5.06 (2H, brs), 7.30 (1H, d, J=8.8 Hz), 7.52 (1H, d, J=8.8 Hz) 7.90 (1H, s), 8.61 (1H, s).

### Preparation of 2-methyl-5-(tributylstannyl)thiazole (Int-2)

To a solution of compound 2-methylthiazole (1.00 g, 10.1 mmol) in anhydrous THF (20 mL) was added n-BuLi (5.25 mL, 13.1 mmol, 2.5M in hexane) dropwise at −78° C. After the completion of addition, the reaction mixture was stirred at −78° C. for 1 hour. Then a solution of tributyltin chloride (4.27 g, 13.1 mmol) in THF (10 mL) was added dropwise to the reaction at −78° C. The reaction mixture was stirred at −78° C. for another 1 hour then at 20° C. for 1 hour to give a yellow mixture. The reaction mixture was quenched with saturated aqueous NaHCO₃ (50 mL) and extracted with EtOAc (50 mL×3). The combined organic layer was dried over anhydrous Na₂SO₄, filtered and concentrated. The residue was purified by flash column (SiO₂, 0% to 30% EtOAc in PE) to give Int-2 (3.80 g, yield: 97%) as colorless oil. ¹H NMR (400 MHz, CDCl₃) δ 0.75-0.85 (10H, m), 1.00-1.05 (5H, m), 1.20-1.30 (6H, m), 1.40-1.55 (6H, m), 2.69 (3H, s), 7.49 (1H, s).

### Preparation of (cyclohexylethynyl)trimethylsilane (Int-3)

To a solution of compound ethynylcyclohexane (1.00 g, 9.24 mmol) in anhydrous THF (20 mL) was added n-BuLi (7.40 mL, 18.5 mmol, 2.5M in hexane) dropwise at −78° C. The reaction mixture was stirred at −78° C. for 0.5 hour, then TMSCl (1.21 g, 11.1 mmol) was added dropwise at −78° C. and the reaction mixture was stirred at 25° C. for 2 hours to give a yellow mixture. The reaction mixture was quenched with saturated aqueous NH₄Cl (25 mL) and extracted with EtOAc (40 mL×4). The combined organic layer was dried over anhydrous Na₂SO₄, filtered and concentrated. The residue was purified by Combi Flash (SiO₂, PE as eluent) to give compound Int-3 (500 mg, yield: 30%) as colorless oil. ¹H NMR (400 MHz, CDCl₃) δ 0.00 (9H, s), 1.08-1.20 (3H, m), 1.23-1.40 (3H, m), 1.50-1.70 (4H, m), 2.15-2.30 (1H, m).

### Preparation of 4-iodo-6-(pyridin-3-yl)isoquinolin-3-amine (Int-4)

### Step 1: Preparation of 6-(pyridin-3-yl)isoquinolin-3-amine

A mixture of 6-bromoisoquinolin-3-amine (550 mg, 2.34 mmol), pyridin-3-ylboronic acid (345 mg, 2.81 mmol), Pd(PPh₃)₄ (271 mg, 0.234 mmol) and Na₂CO₃ (497 mg, 4.68 mmol) in dioxane (10 mL) and water (1 mL) was degassed and purged with N₂ for 3 times. Then the mixture was stirred at 100° C. for 12 hours under N₂ atmosphere. The reaction mixture was diluted with water (50 mL) and extracted with EtOAc (50 mL×2). The combined organic layer was washed with brine (50 mL), dried over anhydrous Na₂SO₄, filtered and concentrated. The residue was purified by Combi Flash (0% to 100% EtOAc in PE) to give 6-(pyridin-3-yl)isoquinolin-3-amine (470 mg, yield: 52%) as a yellow solid. ¹H NMR (400 MHz, DMSO-d₆) δ 6.03 (2H, s), 6.70 (1H, s), 7.46-7.57 (2H, m), 7.87-7.95 (2H, m), 8.16-8.23 (1H, m), 8.62 (1H, dd, J=4.4, 0.8 Hz), 8.86 (1H, s), 9.01 (1H, d, J=2.0 Hz).

### Step 2: Preparation of 4-iodo-6-(pyridin-3-yl)isoquinolin-3-amine (Int-4)

To a solution of 6-(pyridin-3-yl)isoquinolin-3-amine (450 mg, 2.03 mmol) in DMF (5 mL) was added NIS (458 mg, 2.03 mmol) portion-wise at 0° C. The mixture was stirred at 0° C. for 2 hours. The reaction mixture was concentrated and the residue was purified by Combi Flash (0% to 100% EtOAc in PE) to give Int-4 (250 mg, yield: 35%) as a yellow solid. ¹H NMR (400 MHz, DMSO-d₆) δ 6.34 (2H, brs), 7.60-7.70 (2H, m), 7.84 (1H, s), 8.00 (1H, d, J=8.4 Hz), 8.20 (1H, d, J=8.0 Hz), 8.67 (1H, dd, J=4.8, 1.6 Hz), 8.87 (1H, s), 8.99 (1H, d, J=2.0 Hz).

### Preparation of 4-iodo-6-(1-methyl-1H-pyrazol-4-yl)isoquinolin-3-amine (Int-5)

### Step 1: Preparation of 6-(1-methyl-1H-pyrazol-4-yl)isoquinolin-3-amine

A mixture of 6-bromoisoquinolin-3-amine (1.50 g, 6.72 mmol), 1-methyl-4-(4,4,5,5-tetramethyl-1,3,2-dioxaborolan-2-yl)-1H-pyrazole (1.54 g, 7.40 mmol), Pd(dppf)Cl₂ (492 mg, 0.672 mmol), K₂CO₃ (1.86 g, 13.5 mmol) in dioxane (30 mL) and H₂O (6 mL) was degassed and purged with N₂ for 3 times, and then the mixture was stirred at 100° C. for 1 hour under N₂ atmosphere. The reaction mixture was filtered through a pad of celite and the filtrate was concentrated. The residue was purified by Combi Flash (0% to 50% EtOAc in PE) to give 6-(1-methyl-1H-pyrazol-4-yl)isoquinolin-3-amine (930 mg, yield: 47%) as a light brown solid. ¹H NMR (400 MHz, DMSO-d₆) δ 3.88 (3H, s), 5.88 (2H, brs), 6.57 (1H, s), 7.37 (1H, d, J=8.0 Hz), 7.66 (1H, s), 7.75 (1H, d, J=8.8 Hz), 7.99 (1H, s), 8.27 (1H, s), 8.71 (1H, s).

### Step 2: Preparation of 4-iodo-6-(1-methyl-1H-pyrazol-4-yl)isoquinolin-3-amine (Int-5)

To a solution of 6-(1-methyl-1H-pyrazol-4-yl)isoquinolin-3-amine (880 mg, 3.92 mmol) in anhydrous DMF (15 mL) was added NIS (883 mg, 3.92 mmol) at 0° C. The mixture was stirred at 0° C. for 1 hour. The reaction mixture was poured into water (20 mL) at 0° C. and filtered. The solid was collected and purified by Combi Flash (0% to 50% EtOAc in PE) to give compound Int-5 (590 mg, yield: 36%) as a light brown solid. ¹H NMR (400 MHz, CD₃OD) δ 3.96 (3H, s), 7.49 (1H, dd, J=8.0, 1.6 Hz), 7.59 (1H, s), 7.71 (1H, s), 7.76 (1H, d, J=8.4 Hz), 8.12 (1H, s), 8.61 (1H, s).

### Preparation of ((4-(benzyloxy)cyclohexyl)ethynyl)trimethylsilane (Int-6)

### Step 1: Preparation of (((4-(methoxymethylene)cyclohexyl)oxy)methyl)benzene

To a solution of (methoxymethyl)triphenylphosphonium chloride (8.73 g, 25.5 mmol) in anhydrous THF (120 mL) was added LDA (14.7 mL, 29.4 mmol, 2M in THF) dropwise at 0° C. After the completion of the addition, the reaction mixture was stirred at 0° C. for 0.5 hour, the solution was cooled to −65° C. and a solution of 4-(benzyloxy)cyclohexan-1-one (4.00 g, 19.6 mmol) in anhydrous THF (200 mL) was added dropwise to the above solution at −65° C. The mixture was stirred at −65° C. for 1 hour then allowed to stir at 20° C. for 17 hours. The reaction mixture was quenched with water (100 mL) at 25° C. and then extracted with EtOAc (100 mL×3). The combined organic layer was washed with brine (100 mL), dried over anhydrous Na₂SO₄, filtered and concentrated. The residue was purified by Combi Flash (0% to 10% EtOAc in PE) to give (((4-(methoxymethylene)cyclohexyl)oxy)methyl)benzene (3.18 g, yield: 69%) as colorless oil.

### Step 2: Preparation of 4-(benzyloxy)cyclohexane-1-carbaldehyde

To a mixture of (((4-(methoxymethylene)cyclohexyl)oxy)methyl)benzene (3.18 g, 13.7 mmol) in DCM (50 mL) and H₂O (3 mL) was added TFA (4.90 g, 43.0 mmol) at 0° C. The reaction mixture was stirred at 20° C. for 1 hour to give a yellow mixture. The reaction mixture was diluted with H₂O (20 mL) and extracted with DCM (100 mL×3). The combined organic layer was dried over anhydrous Na₂SO₄, filtered and concentrated to give 4-(benzyloxy)cyclohexane-1-carbaldehyde (2.20 g, yield: 73%) as colorless oil. ¹H NMR (400 MHz, CDCl₃) δ 1.46-1.50 (2H, m), 1.56-1.62 (2H, m), 1.75-2.00 (2H, m), 2.02-2.09 (2H, m), 2.15-2.22 (1H, m), 4.49 (2H, s), 7.15-7.20 (1H, m), 7.25-7.35 (4H, m), 9.56 (1H, s).

### Step 3: Preparation of (((4-ethynylcyclohexyl)oxy)methyl)benzene

To a suspension of 4-(benzyloxy)cyclohexane-1-carbaldehyde (2.20 g, 10.1 mmol) and K₂CO₃ (4.18 g, 30.2 mmol) in MeOH (20 mL) was added dimethyl (1-diazo-2-oxopropyl)phosphonate (4.08 g, 20.2 mmol) at 20° C. The mixture was stirred at 20° C. for 16 hours. The reaction mixture was diluted with H₂O (20 mL) and extracted with EtOAc (50 mL×2). The combined organic layer was dried over anhydrous Na₂SO₄, filtered and concentrated.

The residue was purified by Combi Flash (0% to 20% EtOAc in PE) to give (((4-ethynylcyclohexyl)oxy)methyl)benzene (800 mg, yield: 37%) as colorless oil. ¹H NMR (400 MHz, CDCl₃) δ 1.25-1.40 (3H, m), 1.50-1.70 (1H, m), 1.75-1.85 (1H, m), 1.95-2.05 (3H, m), 2.20-2.50 (1H, m), 3.25-3.40 (1H, m), 4.46 (2H, s), 7.15-7.23 (1H, m), 7.25-7.30 (4H, m).

### Step 4: Preparation of ((4-(benzyloxy)cyclohexyl)ethynyl)trimethylsilane (Int-6)

To a solution of (((4-ethynylcyclohexyl)oxy)methyl)benzene (800 mg, 3.73 mmol) in anhydrous THF (20 mL) was added n-BuLi (1.94 mL, 4.85 mmol, 2.5 M in hexane) dropwise at −65° C. After the completion of the addition, the reaction mixture was stirred at −65° C. for 1.5 hours. Then TMSCl (527 mg, 4.85 mmol) was added dropwise to the above solution at −65° C. Then the mixture was allowed to stir at 20° C. for 16.5 hours. The reaction mixture was quenched with water (10 mL) at 20° C. and then diluted with EtOAc (30 mL) and separated. The aqueous layer was extracted with EtOAc (15 mL×2). The combined organic layer was washed with brine (20 mL), dried over anhydrous Na₂SO₄, filtered and concentrated. The residue was purified by Combi Flash (0% to 10% EtOAc in PE) to give Int-6 (970 mg, yield: 90%) as colorless oil. ¹H NMR (400 MHz, CDCl₃) δ 0.08-0.10 (9H, m), 1.25-1.40 (3H, m), 1.60-1.80 (2H, m), 1.90-2.00 (3H, m) 2.25-2.55 (1H, m), 3.28-3.35 (1H, m), 4.47-4.48 (2H, m), 7.20-7.25 (2H, m), 7.27-7.33 (3H, m).

### Preparation of ((3-(benzyloxy)cyclobutyl)ethynyl)trimethylsilane (Int-7)

### Step 1: Preparation of ((3-(methoxymethylene)cyclobutoxy)methyl)benzene

To a mixture of (methoxymethyl)triphenylphosphonium chloride (10.1 g, 29.5 mmol) in anhydrous THF (80 mL) was added LDA (17.0 mL, 34.1 mmol, 2M in THF) dropwise at 0° C. After the completion of the addition, the reaction mixture was stirred at 0° C. for 0.5 hour, then the reaction mixture was cooled to −70° C. and a solution of 3-(benzyloxy)cyclobutan-1-one (4.00 g, 22.7 mmol) in anhydrous THF (150 mL) was added dropwise at −70° C. The reaction mixture was stirred at −70° C. for another 1 hour, then allowed to stir at 20° C. for 16 hours to give a yellow mixture. The reaction mixture was poured into brine (100 mL) and extracted with EtOAc (100 mL×3). The combined organic layer was dried over anhydrous Na₂SO₄, filtered and concentrated. The residue was purified by Combi Flash (SiO₂, 0% to 20% EtOAc in PE) to give ((3-(methoxymethylene)cyclobutoxy)methyl)benzene (4.10 g, yield: 88%) as colorless oil. ¹H NMR (400 MHz, CDCl₃) δ 2.65-2.75 (2H, m), 2.80-2.90 (1H, m), 2.95-3.05 (1H, m), 3.58 (3H, s), 4.10-4.20 (1H, m), 4.49 (2H, s), 5.90 (1H, s), 7.29-7.40 (5H, m).

### Step 2: Preparation of 3-(benzyloxy)cyclobutane-1-carbaldehyde

To a mixture of ((3-(methoxymethylene)cyclobutoxy)methyl)benzene (1.00 g, 4.90 mmol) in DCM (25 mL) and H₂O (1.5 mL) was added TFA (1.54 g, 13.5 mmol) at 0° C. The reaction mixture was stirred at 0° C. for 1 hour to give a yellow mixture. The reaction mixture was poured into brine (30 mL) and extracted with DCM (50 mL×4). The combined organic layer was dried over anhydrous Na₂SO₄, filtered and concentrated to give 3-(benzyloxy)cyclobutane-1-carbaldehyde (1.00 g, crude) as colorless oil. ¹H NMR (400 MHz, CDCl₃) δ 2.10-2.25 (2H, m), 2.30-2.40 (1H, m), 2.40-2.50 (1H, m), 2.60-3.05 (1H, m), 3.95-4.05 (1H, m), 4.35-4.45 (2H, m), 7.20-7.30 (5H, m), 9.55-9.75 (1H, m).

### Step 3: Preparation of ((3-ethynylcyclobutoxy)methyl)benzene

To a mixture of 3-(benzyloxy)cyclobutane-1-carbaldehyde (1.00 g, 5.26 mmol) and dimethyl (1-diazo-2-oxopropyl)phosphonate (2.02 g, 10.5 mmol) in MeOH (15 mL) was added K₂CO₃ (2.18 g, 15.8 mmol) at 0° C. The reaction mixture was stirred at 20° C. for 16 hours to give a yellow mixture. The reaction mixture was filtered and the filtrate was concentrated. The residue was purified by Combi Flash (SiO₂, 0% to 15% EtOAc in PE) to give ((3-ethynylcyclobutoxy)methyl)benzene (540 mg, yield: 55% for 2 steps) as colorless oil. ¹H NMR (400 MHz, CDCl₃) δ 2.00-2.10 (2H, m), 2.20-2.35 (2H, m), 2.37-3.05 (2H, m), 3.75-4.30 (1H, m), 4.34 (2H, s), 7.20-7.30 (5H, m).

### Step 4: Preparation of ((3-(benzyloxy)cyclobutyl)ethynyl)trimethylsilane

To a mixture of ((3-ethynylcyclobutoxy)methyl)benzene (540 mg, 2.90 mmol) in anhydrous THF (10 mL) was added n-BuLi (1.51 mL, 3.78 mmol, 2.5M in hexane) dropwise at −70° C. After the completion of the addition, the mixture was stirred at −70° C. for 1 hour.

TMSCl (409 mg, 3.77 mmol) was added to the mixture and the resulting reaction mixture was stirred at 20° C. for 4 hours to give a yellow mixture. The reaction mixture was poured into brine (20 mL) and extracted with EtOAc (20 mL×4). The combined organic layer was dried over anhydrous Na₂SO₄, filtered and concentrated. The residue was purified by Combi Flash (SiO₂, 0% to 15% EtOAc in PE) to give Int-7 (690 mg, yield: 92%) as colorless oil. ¹H NMR (400 MHz, CDCl₃) δ 0.16 (9H, s), 2.10-2.20 (1H, m), 2.25-3.10 (4H, m), 3.90-4.40 (1H, m), 4.42 (3H, s), 7.30-7.40 (5H, m).

Preparation of methyl 4-((trimethylsilyl)ethynyl)cyclohexane-1-carboxylate (Int-8)

### Step 1: Preparation of methyl 4-(methoxymethylene)cyclohexane-1-carboxylate

To a solution of (methoxymethyl)triphenylphosphonium chloride (25.7 g, 74.9 mmol) in anhydrous THF (150 mL) was added LDA (43.2 mL, 86.4 mmol, 2M in THF) dropwise at 0° C. After stirring at 0° C. for 0.5 hour, the reaction mixture was cooled to −70° C. and a solution of methyl 4-oxocyclohexane-1-carboxylate (9.00 g, 57.6 mmol) in anhydrous THF (150 mL) was added dropwise at −70° C. After the completion of the addition, the reaction mixture was stirred at −70° C. for 1 hour and then allowed to stir at 20° C. for 14.5 hours. The reaction mixture was quenched with H₂O (30 mL) and concentrated. The residue was poured into water (100 mL) and extracted with EtOAc (100 mL×3), the combined organic layer was washed with brine (150 mL), dried over anhydrous Na₂SO₄, filtered and concentrated. The residue was purified by Combi Flash (ISCO®; 40 g SepaFlash® Silica Flash Column, Eluent of 0˜8% Ethyl acetate/Petroleum ether gradient @55 mL/min) to give methyl 4-(methoxymethylene)cyclohexane-1-carboxylate (9.20 g, yield: 87%) as colorless oil. ¹H NMR (400 MHz, CDCl₃) δ 1.40-1.55 (2H, m), 1.70-1.82 (1H, m), 1.86-2.02 (3H, m), 2.07-2.17 (1H, m), 2.37-2.49 (1H, m), 2.69-2.80 (1H, m), 3.54 (3H, s), 3.67 (3H, s), 5.78 (1H, s).

### Step 2: Preparation of methyl 4-formylcyclohexane-1-carboxylate

To a solution of methyl 4-(methoxymethylene)cyclohexane-1-carboxylate (9.20 g, 49.9 mmol) in DCM (280 mL) and H₂O (20 mL) was added TFA (14.2 g, 125 mmol) at 0° C., the mixture was stirred at 0° C. for 1 hour and then stirred at 20° C. for 2 hours. The reaction mixture was concentrated and the residue was poured into water (150 mL) and extracted with DCM (100 mL×2), the combined organic layer was dried over anhydrous Na₂SO₄, filtered and concentrated to give methyl 4-formylcyclohexane-1-carboxylate (8.40 g, yield: 99%) as light yellow oil. ¹H NMR (400 MHz, CDCl₃) δ 1.22-1.38 (1H, m), 1.44-1.57 (1H, m), 1.64-1.80 (3H, m), 1.90-2.00 (1H, m), 2.05-2.15 (2H, m), 2.19-2.33 (1H, m), 2.34-2.51 (1H, m), 3.65-3.71 (3H, m), 9.60-9.69 (1H, m).

### Step 3: Preparation of methyl 4-ethynylcyclohexane-1-carboxylate

To a solution of methyl 4-formylcyclohexane-1-carboxylate (8.40 g, 49.4 mmol) and dimethyl (1-diazo-2-oxopropyl)phosphonate (20.9 g, 109 mmol) in absolute MeOH (150 mL) was added K₂CO₃ (20.5 g, 148 mmol) at 0° C., the mixture was stirred at 20° C. for 12 hours. The reaction mixture was concentrated and the residue was poured into H₂O (80 mL) and extracted with EtOAc (80 mL×2). The combined organic layer was dried over anhydrous Na₂SO₄, filtered and concentrated. The residue was purified by Combi Flash (ISCO®; 40 g SepaFlash® Silica Flash Column, Eluent of 0˜6% Ethyl acetate/Petroleum ether gradient @50 mL/min) to give methyl 4-ethynylcyclohexane-1-carboxylate (5.10 g, yield: 61%) as light yellow oil.

¹H NMR (400 MHz, CDCl₃) δ 1.35-1.50 (2H, m), 1.56-1.63 (1H, m), 1.69-1.88 (2H, m), 1.88-2.09 (4H, m), 2.25-2.75 (2H, m), 3.65-3.72 (3H, m).

### Step 4: Preparation of methyl 4-((trimethylsilyl)ethynyl)cyclohexane-1-carboxylate (Int-8)

To a solution of methyl 4-ethynylcyclohexane-1-carboxylate (5.10 g, 30.7 mmol) in anhydrous THF (100 mL) was added LDA (16.1 mL, 32.2 mmol, 2M in THF) dropwise at −70° C., the mixture was stirred at −70° C. for 1.5 hours. TMSCl (5.00 g, 46.0 mmol) was added to the mixture at −70° C. and stirred at −70° C. for 0.5 hour and then allowed to stir at 20° C. for 2 hours under N₂ atmosphere. The reaction mixture was quenched with H₂O (20 mL) and concentrated. The residue was poured into H₂O (100 mL) and extracted with EtOAc (100 mL×2). The combined organic layer was dried over anhydrous Na₂SO₄, filtered and concentrated. The residue was purified by Combi Flash (ISCO®; 40 g SepaFlash® Silica Flash Column, Eluent of 0-7% Ethyl acetate/Petroleum ether gradient @40 mL/min) to give Int-8 (4.20 g, yield: 57%) as light yellow oil. ¹H NMR (400 MHz, CDCl₃) δ 0.10-0.20 (9H, m), 1.30-1.60 (3H, m), 1.70-1.80 (2H, m), 1.85-2.10 (3H, m), 2.25-2.75 (2H, m), 3.60-3.75 (3H, m).

### Preparation of tert-butyl 4-((trimethylsilyl)ethynyl)piperidine-1-carboxylate (Int-9)

To a solution of tert-butyl 4-ethynylpiperidine-1-carboxylate (500 mg, 2.39 mmol) in anhydrous THF (10 mL) was added n-BuLi (1.24 mL, 3.10 mmol, 2.5M in hexane) dropwise at −70° C. After stirring at −70° C. for 1.5 hours, TMSCl (337 mg, 3.11 mmol) was added at -70° C. and the mixture was stirred at 20° C. for 4 hours. The reaction mixture was quenched with H₂O (10 mL) at 20° C. and extracted with EtOAc (10 mL×2). The combined organic layer was washed with brine (10 mL), dried over anhydrous Na₂SO₄, filtered and concentrated. The residue was purified by Combi Flash (ISCO®; 4 g SepaFlash® Silica Flash Column, Eluent of 0-20% Ethyl acetate/Petroleum ether gradient @40 mL/min) to give Int-9 (660 mg, yield: 98%) as colorless oil. ¹H NMR (400 MHz, CDCl₃) δ 0.15 (9H, s), 1.46 (9H, s), 1.50-1.60 (2H, m), 1.70-1.80 (2H, m), 2.55-2.65 (1H, m), 3.10-3.30 (2H, m), 3.55-3.80 (2H, m).

### Preparation of 6-(4,4,5,5-tetramethyl-1,3,2-dioxaborolan-2-yl)benzo[d]isothiazole (Int-10)

### Step 1: Preparation of 2-(benzylthio)-4-bromobenzaldehyde

To a solution of BnSH (3.36 g, 27.1 mmol) in THF (50 mL) was added t-BuOK (3.04 g, 27.1 mmol). The mixture was stirred at 25° C. for 0.2 hour. Then a solution of 4-bromo-2-fluorobenzaldehyde (5.00 g, 24.6 mmol) in THF (50 mL) was added to the mixture and stirred at 25° C. for 0.8 hour. The reaction mixture was concentrated and the residue diluted with H₂O (80 mL) and extracted with EtOAc (100 mL×3). The combined organic layers were washed with brine (200 mL), dried over anhydrous Na₂SO₄, filtered and concentrated. The residue was purified by silica gel column (0% to 10% EtOAc in PE) to give 2-(benzylthio)-4-bromobenzaldehyde (5.50 g, yield: 73%) as a yellow solid. ¹H NMR (400 MHz, CDCl₃) δ 4.07 (2H, s), 7.20-7.29 (5H, m), 7.38 (1H, dd, J=8.0, 1.6 Hz), 7.52 (1H, d, J=1.6 Hz), 7.59 (1H, d, J=8.4 Hz), 10.10 (1H, s).

### Step 2: Preparation of 6-bromobenzo[d]isothiazole

To a solution of 2-(benzylthio)-4-bromobenzaldehyde (5.50 g, 17.9 mmol) in DCE (50 mL) was added SO₂Cl₂ (2.42 g, 17.9 mmol). The mixture was stirred at 25° C. for 1 hour. The reaction mixture was concentrated and the residue was dissolved in THF (50 mL), NH₃ (7.7 mL, 7M in CH₃OH) was added, then the resulting mixture was stirred at 25° C. for 11 hours.

The reaction mixture was concentrated and the residue was diluted with H₂O (50 mL) and extracted with EtOAc (80 mL×3). The combined organic layers were washed with brine (100 mL), dried over anhydrous Na₂SO₄, filtered and concentrated. The residue was purified by silica gel column (0% to 20% EtOAc in PE) to give 6-bromobenzo[d]isothiazole (3.00 g, yield: 78%) as a yellow solid. ¹H NMR (400 MHz, CDCl₃) δ 7.57 (1H, dd, J=8.4, 1.6 Hz), 7.94 (1H, d, J=8.4 Hz), 8.15 (1H, s), 8.88 (1H, s).

### Step 3: Preparation of 6-(4,4,5,5-tetramethyl-1, 3,2-dioxaborolan-2-yl)benzo[d]isothiazole (Int-10)

To a solution of 6-bromobenzo[d]isothiazole (3.00 g, 14.0 mmol), Bis-Pin (4.63 g, 18.2 mmol) in dioxane (50 mL) was added KOAc (2.06 g, 21.0 mmol), PCy₃ (432 mg, 1.54 mmol) and Pd₂(dba)₃ (642 mg, 0.700 mmol), then the mixture was degassed and purged with N₂ for 3 times and the mixture was stirred at 110° C. for 12 hours under N₂ atmosphere. The reaction mixture was suspended in CH₃OH (50 mL) and filtered. The filtrate was concentrated and the residue was purified by silica gel column (0% to 10% EtOAc in PE) to give Int-10 (3.00 g, yield: 82%) as a yellow solid.

Preparation of methyl 3-((trimethylsilyl)ethynyl)cyclobutane-1-carboxylate (Int-11)

### Step 1: Preparation of methyl 3-(methoxymethylene)cyclobutane-1-carboxylate

To a solution of (methoxymethyl)triphenylphosphonium chloride (34.7 g, 101 mmol) in anhydrous THF (300 mL) was added LDA (58.5 mL, 117 mmol, 2M in THF) dropwise at 0° C. After the completion of the addition, the reaction mixture was stirred at 0° C. for 0.5 hour, then cooled to −65° C. and a solution of methyl 3-oxocyclobutane-1-carboxylate (10.0 g, 78.1 mmol) in anhydrous THF (200 mL) was added dropwise to the above solution at −65° C. The mixture was stirred at −65° C. for 1 hour, then slowly warmed to 25° C. and stirred at 25° C. for 17 hours. The reaction mixture was quenched with water (200 mL) and extracted with EtOAc (100 mL×3). The combined organic layer was dried over anhydrous Na₂SO₄, filtered and concentrated. The residue was purified by Combi Flash (0% to 15% EtOAc in PE) to give methyl 3-(methoxymethylene)cyclobutane-1-carboxylate (9.00 g, yield: 74%) as colorless oil. ¹H NMR (400 MHz, CDCl₃) δ 2.80-3.00 (4H, m), 3.10-3.25 (1H, m), 3.55 (3H, s), 3.70 (3H, s), 5.80-5.90 (1H, m).

### Step 2: Preparation of methyl 3-formylcyclobutane-1-carboxylate

To a mixture of methyl 3-(methoxymethylene)cyclobutane-1-carboxylate (5.00 g, 32.0 mmol) in DCM (72 mL) and water (4.2 mL) was added TFA (8.39 g, 73.6 mmol, 5.45 mL) at 0° C. The reaction mixture was stirred at 25° C. for 2 hours to give a yellow mixture. The reaction mixture was concentrated and the residue was diluted with water (50 mL), then extracted with DCM (50 mL×2). The combined organic layer was dried over anhydrous Na₂SO₄, filtered and concentrated to give methyl 3-formylcyclobutane-1-carboxylate (3.70 g, crude) as colorless oil.

### Step 3: Preparation of methyl 3-ethynylcyclobutane-1-carboxylate

To a solution of methyl 3-formylcyclobutane-1-carboxylate (3.70 g, 26.0 mmol) and K₂CO₃ (7.19 g, 52.1 mmol) in MeOH (50 mL) was added dimethyl (1-diazo-2-oxopropyl)phosphonate (7.50 g, 39.0 mmol). The mixture was stirred at 20° C. for 16 hours. The reaction mixture was concentrated and the residue was diluted with water (50 mL) and extracted with EtOAc 100 mL (50 mL×2). The combined organic layer was dried over anhydrous Na₂SO₄, filtered and concentrated. The residue was purified by Combi Flash (0% to 15% EtOAc in PE) to give methyl 3-ethynylcyclobutane-1-carboxylate (1.10 g, yield: 31% for 2 steps) as colorless oil. ¹H NMR (400 MHz, CDCl₃) δ 2.20-2.25 (1H, m), 2.35-2.50 (2H, m), 2.50-2.65 (2H, m), 2.90-3.30 (2H, m), 3.65-3.75 (3H, m).

### Step 4: Preparation of methyl 3-((trimethylsilyl)ethynyl)cyclobutane-1-carboxylate (Int-11)

To a solution of methyl 3-ethynylcyclobutane-1-carboxylate (1.10 g, 7.96 mmol) in anhydrous THF (40 mL) was added LDA (4.18 mL, 9.36 mmol, 2M in THF) dropwise at -65° C., the mixture was stirred at −65° C. for 1.5 hours. TMSCl (1.30 g, 11.9 mmol) was added dropwise to the mixture at −65° C. After the completion of the addition, the reaction mixture was stirred at −65° C. for 0.5 hour and then at 25° C. for 2 hours under N₂ atmosphere. The reaction mixture was quenched with water (50 mL) at 25° C. and then extracted with EtOAc (50 mL×2). The combined organic layer was dried over anhydrous Na₂SO₄, filtered and concentrated. The residue was purified by Combi Flash (0% to 15% EtOAc in PE) to give Int-11 (1.00 g, yield: 60%) as colorless oil. ¹H NMR (400 MHz, CDCl₃) δ 0.00-0.06 (9H, m), 2.15-2.30 (2H, m), 2.30-2.45 (2H, m), 2.75-3.20 (2H, m), 3.50-3.60 (3H, m).

### Preparation of 5-iodo-1, 7-naphthyridin-6-amine (Int-12)

### Step 1: Preparation of 8-bromo-1, 7-naphthyridin-6-amine

A solution of 3-(cyanomethyl)picolinonitrile (800 mg, 5.59 mmol) in HBr (8 mL, 33% in HOAc) was stirred at 20° C. for 1 hour. The reaction mixture was diluted with EtOAc (20 mL) and adjusted pH=7 with saturated aqueous NaHCO₃ (20 mL) at 20° C., then extracted with EtOAc (20 mL×2). The combined organic layer was washed with brine (20 mL), dried over anhydrous Na₂SO₄, filtered and concentrated to give 8-bromo-1,7-naphthyridin-6-amine (1.08 g, yield: 86%) as a light green solid. ¹H NMR (400 MHz, DMSO-d₆) δ 6.48 (2H, brs), 6.60 (1H, s), 7.48 (1H, dd, J=8.4, 4.0 Hz), 8.02 (1H, dd, J=8.4, 1.2 Hz), 8.59 (1H, d, J=2.0 Hz).

### Step 2: Preparation of 1, 7-naphthyridin-6-amine

A mixture of 8-bromo-1,7-naphthyridin-6-amine (1.08 g, 4.82 mmol), 10% Pd/C (180 mg, 50% H₂O wt/wt) and NaOH (231 mg, 5.78 mmol) in MeOH (15 mL) was degassed and purged with H₂ for 3 times, then the mixture was hydrogenated (15 psi) at 20° C. for 16 hours. The reaction mixture was filtered through a pad of celite and the filtrate was concentrated to give 1, 7-naphthyridin-6-amine (700 mg, crude) as a yellow solid. ¹H NMR (400 MHz, DMSO-d₆) δ 6.16 (2H, brs), 6.61 (1H, s), 7.41 (1H, dd, J=8.4, 4.0 Hz), 7.96 (1H, d, J=8.4 Hz), 8.52 (1H, dd, J=4.0, 1.6 Hz), 8.87 (1H, s).

### Step 3: Preparation of 5-iodo-1, 7-naphthyridin-6-amine (Int-12)

To a solution of 1,7-naphthyridin-6-amine (700 mg, 4.82 mmol) in anhydrous DMF (10 mL) was added NIS (1.08 g, 4.82 mmol) at 20° C. The mixture was stirred at 20° C. for 1 hour. The reaction mixture was slowly poured into water (10 mL) at 20° C. and filtered. The solid was collected and purified by Combi Flash (0% to 30% EtOAc in PE) to give Int-12 (590 mg, yield: 45% for 2 steps) as a light yellow solid. ¹H NMR (400 MHz, DMSO-d₆) o 6.44 (2H, brs), 7.61 (1H, dd, J=8.4, 4.0 Hz), 8.00-8.05 (1H, m), 8.61 (1H, dd, J=4.0, 1.2 Hz), 8.87 (1H, s).

### Preparation of 4-iodo-2,6-naphthyridin-3-amine (Int-13)

### Step 1: Preparation of tert-butyl 2-cyano-2-(4-cyanopyridin-3-yl)acetate

A mixture of 3-bromoisonicotinonitrile (5.00 g, 27.3 mmol), tert-butyl cyanoacetate (5.01 g, 35.5 mmol), Pd(dppf)Cl₂ (2.00 g, 2.73 mmol) and t-BuOK (6.44 g, 57.4 mmol) in anhydrous dioxane (100 mL) was degassed and purged with N₂ for 3 times. Then the reaction mixture was stirred at 70° C. for 3 hours. The reaction mixture was quenched with saturated aqueous NH₄Cl (100 mL) and extracted with EtOAc (100 mL×3). The combined organic layer was washed with brine (500 mL), dried over anhydrous Na₂SO₄, filtered and concentrated. The residue was purified by silica gel column chromatography (PE/EtOAc=2/1 to 1/1) to give tert-butyl 2-cyano-2-(4-cyanopyridin-3-yl)acetate (2.80 g, yield: 42%) as yellow oil. ¹H NMR (400 MHz, CDCl₃) δ 1.35-1.45 (9H, m), 4.95-5.00 (1H, m), 7.50-7.60 (1H, m), 8.75-8.85 (1H, m), 8.94 (1H, s).

### Step 2: Preparation of 1-bromo-2,6-naphthyridin-3-amine

A solution of tert-butyl 2-cyano-2-(4-cyanopyridin-3-yl)acetate (2.50 g, 10.3 mmol) in HBr (10 mL, 33% in HOAc) was stirred at 25° C. for 0.5 hour. The reaction mixture was basified with saturated aqueous NaHCO₃ to pH=8 and extracted with EtOAc (100 mL×3). The combined organic layer was washed with brine (20 mL), dried over anhydrous Na₂SO₄, filtered and concentrated. The residue was purified by silica gel column (PE/EtOAc =2/1 to 1/1) to give 1-bromo-2,6-naphthyridin-3-amine (700 mg, yield: 30%) as a yellow solid.

¹H NMR (400 MHz, CDCl₃) δ 4.64 (2H, brs), 6.65-6.70 (1H, m), 7.70 (1H, d, J=6.0 Hz), 8.33 (1H, d, J=6.0 Hz), 8.95 (1H, s).

### Step 3: Preparation of 2,6-naphthyridin-3-amine

To a solution of 1-bromo-2,6-naphthyridin-3-amine (700 mg, 3.12 mmol) in MeOH (20 mL) was added 10% Pd/C (400 mg, 50% moisture wt/wt) and NaOH (125 mg, 3.12 mmol) under N₂ atmosphere. The mixture was degassed and purged with H₂ for 3 times and then hydrogenated (15 psi) at 25° C. for 2 hours. The reaction mixture filtered and the filtrate was concentrated. To the residue was added H₂O (20 mL) and extracted with EtOAc (60 mL×3). The combined organic layer was washed with brine (15 mL), dried over anhydrous Na₂SO₄, filtered and concentrated to give 2,6-naphthyridin-3-amine (400 mg, yield: 61%) as a yellow solid. ¹H NMR (400 MHz, DMSO-d₆) δ 6.28 (2H, brs), 6.73 (1H, s), 7.66 (1H, d, J=5.6 Hz), 8.17 (1H, d, J=5.6 Hz), 8.93 (1H, s), 9.02 (1H, s).

### Step 4: Preparation of 4-iodo-2,6-naphthyridin-3-amine (Int-13)

To a solution of 2,6-naphthyridin-3-amine (200 mg, 1.38 mmol) in DMF (2 mL) was added NIS (372 mg, 1.65 mmol) at 25° C. . The mixture was stirred at 25° C. for 0.5 hour. The reaction mixture was quenched with water (15 mL) and extracted with EtOAc (40 mL×3). The combined organic layer was washed with brine (15 mL) and dried over anhydrous Na₂SO₄, filtered and concentrated. The residue was purified by Combi Flash (0% to 30% EtOAc in PE) to give Int-13 (100 mg, yield: 27%) as yellow oil. ¹H NMR (400 MHz, CD₃OD) δ 7.58 (1H, d, J=5.6 Hz), 8.22 (1H, d, J=5.6 Hz), 8.77 (1H, s), 8.99 (1H, s).

### Preparation of 8-iodo-1,6-naphthyridin-7-amine (Int-14)

### Step 1: Preparation of 2-bromo-5-iodopyridin-4-amine

A solution of 2-bromopyridin-4-amine (13.0 g, 75.1 mmol) and NIS (20.3 g, 90.2 mmol) in CH₃CN (200 mL) was stirred at 70° C. for 12 hours. The reaction mixture was concentrated and the residue was diluted with H₂O (100 mL) and extracted with EtOAc (200 mL×3). The combined organic layer was washed with brine (100 mL), dried over anhydrous Na₂SO₄, filtered and concentrated. The residue was purified by Combi Flash (0% to 12% EtOAc in PE) to give 2-bromo-5-iodopyridin-4-amine (3.60 g, yield: 16%) as a yellow solid. ¹H NMR (400 MHz, DMSO-d₆) δ 6.51 (2H, brs), 6.78 (1H, s), 8.17 (1H, s).

### Step 2: Preparation of ethyl (E)-3-(4-amino-6-bromopyridin-3-yl)acrylate

A mixture of 2-bromo-5-iodopyridin-4-amine (3.60 g, 12.0 mmol), prop-2-enoate (2.41 g, 24.1 mmol), TEA (1.83 g, 18.1 mmol), tri-o-tolylphosphine (293 mg, 0.963 mmol) and Pd(OAc)₂ (108 mg, 0.482 mmol) in DMF (35 mL) was degassed and purged with N₂ for 3 times and then the mixture was stirred at 100° C. for 3 hours under N₂ atmosphere. The reaction mixture was suspended in EtOAc (200 mL) and filtered to remove the insoluble. The filtrate was washed with H₂O (50 mL×2), dried over anhydrous Na₂SO₄, filtered and concentrated. The residue was purified by Combi Flash (0% to 30% EtOAc in PE) to give ethyl (E)-3-(4-amino-6-bromopyridin-3-yl)acrylate (2.30 g, yield: 70%) as a yellow solid. ¹H NMR (400 MHz, DMSO-d₆) δ 1.26 (3H, t, J=6.8 Hz), 4.19 (2H, q, J=6.8 Hz), 6.53 (1H, d, J=15.6 Hz), 6.77 (1H, s), 6.81 (2H, brs), 7.74 (1H, d, J=16.0 Hz), 8.24 (1H, s).

### Step 3: Preparation of 7-bromo-1,6-naphthyridin-2(JH)-one

A solution of ethyl (E)-3-(4-amino-6-bromopyridin-3-yl)acrylate (2.20 g, 8.11 mmol) and n-Bu₃P (1.64 g, 8.11 mmol) in HOAc (20 mL) was stirred at 110° C. for 1 hour. The reaction mixture was filtered and the solid was washed with EtOAc (10 mL×2) and dried to give 7-bromo-1,6-naphthyridin-2(1H)-one (1.50 g, yield: 78%) as a yellow solid. ¹H NMR (400 MHz, DMSO-d₆) δ 6.62 (1H, d, J=9.6 Hz), 7.37 (1H, s), 8.00 (1H, d, J=9.6 Hz), 8.66 (1H, s), 12.10 (1H, brs).

### Step 4: Preparation of 7-bromo-2-chloro-1,6-naphthyridine

To a solution of 7-bromo-1,6-naphthyridin-2(1H)-one (1.50 g, 6.67 mmol) in POCl₃ (49.5 g, 323 mmol) was added DMF (49 mg, 0.667 mmol). Then the reaction mixture was stirred at 80° C. for 2 hours. The reaction mixture was concentrated and the residue was quenched with H₂O (50 mL) and neutralized with 1M aqueous NaOH to pH=7 and filtered. The solid was washed with H₂O (10 mL×2) and dried in vacuum to give 7-bromo-2-chloro-1,6-naphthyridine (1.50 g, yield: 92%) as a yellow solid. ¹H NMR (400 MHz, DMSO-d₆) δ 7.83 (1H, d, J=8.4 Hz), 8.23 (1H, s), 8.69 (1H, dd, J=8.8 Hz), 9.32 (1H, s).

### Step 5: Preparation of 7-bromo-1,6-naphthyridine

A solution of 7-bromo-2-chloro-1,6-naphthyridine (1.50 g, 6.16 mmol), Pd(PPh₃)₄ (570 mg, 0.493 mmol) in anhydrous toluene (30 mL) was degassed and purged with N₂ for 3 times. Then n-Bu₃SnH (1.79 g, 6.16 mmol) was added to the reaction mixture and the resulting reaction mixture was stirred at 25° C. under N₂ atmosphere for 16 hours. The reaction mixture was diluted with EtOAc (100 mL) and washed with H₂O (50 mL×3), dried over anhydrous Na₂SO₄, filtered and concentrated. The residue was purified by Combi Flash (0% to 25% EtOAc in PE) to give 7-bromo-1,6-naphthyridine (800 mg, yield: 62%) as a yellow solid. ¹H NMR (400 MHz, DMSO-d₆) δ 7.76 (1H, dd, J=8.0, 4.0 Hz), 8.23 (1H, s), 8.64 (1H, d, J=8.4 Hz), 9.17 (1H, dd, J=4.0, 1.6 Hz), 9.29 (1H, s).

### Step 6: Preparation of tert-butyl (1,6-naphthyridin-7-yl)carbamate

A mixture of 7-bromo-1,6-naphthyridine (800 mg, 3.83 mmol), BocNH₂ (493 mg, 4.21 mmol), Pd₂(dba)₃ (350 mg, 0.383 mmol), Xantphos (443 mg, 0.765 mmol) and Cs₂CO₃ (3.74 g, 11.5 mmol) in anhydrous dioxane (10 mL) was degassed and purged with N₂ for 3 times and then the mixture was stirred at 100° C. for 12 hours under N₂ atmosphere. The reaction mixture was suspended in MeOH (10 mL) and filtered to remove the insoluble. The filtrate was concentrated and the residue was purified by Combi Flash (0% to 20% EtOAc in PE) to give tert-butyl (1,6-naphthyridin-7-yl)carbamate (400 mg, yield: 42%) as a yellow solid. ¹H NMR (400 MHz, DMSO-d₆) δ 1.52 (9H, s), 7.50 (1H, dd, J=8.4, 4.4 Hz), 8.23 (1H, s), 8.45 (1H, d, J=8.0 Hz), 9.02 (1H, dd, J=4.4, 1.6 Hz), 9.16 (1H, s), 10.09 (1H, brs).

### Step 7: Preparation of 1,6-naphthyridin-7-amine

To a solution of tert-butyl (1,6-naphthyridin-7-yl)carbamate (400 mg, 1.63 mmol) in DCM (4 mL) was added TFA (4 mL) and the reaction mixture was stirred at 25° C. for 2 hours to give a yellow mixture. The reaction mixture was concentrated and the residue was diluted with H₂O (10 mL) and extracted with EtOAc (20 mL×3). The combined organic layer was washed with brine (20 mL), dried over anhydrous Na₂SO₄, filtered and concentrated to give 1,6-naphthyridin-7-amine (200 mg, yield: 84%) as a yellow solid. ¹H NMR (400 MHz, DMSO-d₆) δ 6.29 (2H, brs), 6.71 (1H, s), 7.14 (1H, dd, J=8.4, 4.4 Hz), 8.19 (1H, dd, J=8.4, 1.2 Hz), 8.75 (1H, dd, J=4.4, 2.0 Hz), 8.89 (1H, s).

### Step 8: Preparation of 8-iodo-1,6-naphthyridin-7-amine (Int-14)

To a solution of 1,6-naphthyridin-7-amine (200 mg, 1.38 mmol) in DMF (4 mL) was added NIS (341 mg, 1.52 mmol). Then the reaction mixture was stirred at 25° C. for 12 hours.

The reaction mixture was diluted with H₂O (20 mL) and extracted with EtOAc (30 mL×3).

The combined organic layer was washed with brine (30 mL), dried over anhydrous Na₂SO₄, filtered and concentrated. The residue was purified by Combi Flash (0% to 30% EtOAc in PE) to give Int-14 (330 mg, yield: 88%) as a yellow solid. ¹H NMR (400 MHz, DMSO-d₆) δ 6.53 (2H, brs), 7.29 (1H, dd, J=8.4, 4.4 Hz), 8.24 (1H, d, J=8.0 Hz), 8.83 (1H, s), 8.87 (1H, dd, J=4.40, 2.4 Hz).

### Preparation of tert-butyl 4-(prop-1-yn-1-yl)piperidine-1-carboxylate (Int-15)

To a solution of tert-butyl 4-ethynylpiperidine-1-carboxylate (1.00 g, 4.78 mmol) in anhydrous THF (15 mL) was added n-BuLi (2.48 mL, 6.20 mmol, 2.5M in hexane) dropwise at −65° C. After the addition, the mixture was warmed up to 25° C. slowly and then MeI (1.02 g, 7.17 mmol) was added dropwise at 25° C. The resulting mixture was stirred at 25° C. for 12 hours. The reaction mixture was quenched with H₂O (25 mL) and then extracted with EtOAc (50 mL×2). The combined organic layer was dried over anhydrous Na₂SO₄, filtered and concentrated. The residue was purified by Combi Flash (0% to 6% EtOAc in PE) to give Int-15 (770 mg, yield: 72%) as a colorless oil. ¹H NMR (400 MHz, CDCl₃) δ 1.38 (s, 9H), 1.40-1.50 (2H, m), 1.60-1.70 (2H, m), 1.73 (3H, s), 2.40-2.50 (1H, m), 3.00-3.10 (2H, m), 3.55-3.65 (2H, m).

### Preparation of 4-((trimethylsilyl)ethynyl)pyridine (Int-16)

A mixture of 4-bromopyridine (4.00 g, 25.3 mmol), CuI (145 mg, 0.759 mmol), Pd(PPh₃)₂Cl₂ (888 mg, 1.27 mmol) and TEA (7.69 g, 76.0 mmol) in DMF (65 mL) was degassed and purged with N₂ for three times. Ethynyltrimethylsilane (3.73 g, 38.0 mmol) was added and the resulting mixture was stirred at 25° C. for 16 hours under N₂ atmosphere. The reaction mixture was filtered and the filtrated was concentrated. The residue was purified by flash silica gel chromatography (ISCO®; 40 g SepaFlash® Silica Flash Column, Eluent of ˜10% Ethyl acetate/Petroleum ether gradient @50 mL/min) to give Int-16 (2.50 g, yield: 56%) as yellow oil.

### Preparation of 4-(3-methyl-8-(1-methyl-1H-pyrazol-4-yl)-3H-pyrrolo[2,3-c]isoquinolin-1-yl)cyclohexan-1-ol (Int-17)

### Step 1: Preparation of 1-(4-(benzyloxy)cyclohexyl)-3-methyl-8-(1-methyl-1H-pyrazol-4-yl)-3H-pyrrolo[2,3-c]isoquinoline

To a solution of 1-(4-(benzyloxy)cyclohexyl)-8-(1-methyl-1H-pyrazol-4-yl)-3H-pyrrolo[2,3-c]isoquinoline (330 mg, 0.755 mmol) in anhydrous DMF (5 mL) was added NaH (60 mg, 1.51 mmol, 60% dispersion in mineral oil) at 0° C., then MeI (160 mg, 1.13 mmol) was added dropwise to the above solution. The mixture was stirred at 0° C. for 1 hour. The reaction mixture was quenched by addition MeOH (2 mL) at 0° C., then concentrated. The residue was purified by Combi Flash (0% to 50% EtOAc in PE) to give 1-(4-(benzyloxy)cyclohexyl)-3-methyl-8-(1-methyl-1H-pyrazol-4-yl)-3H-pyrrolo[2,3-c]isoquinoline (440 mg, yield: 99%) as yellow oil.

### Step 2: Preparation of 4-(3-methyl-8-(1-methyl-1H-pyrazol-4-yl)-3H-pyrrolo[2,3-c]isoquinolin-1-yl)cyclohexan-1-ol (Int-17)

A mixture of 1-(4-(benzyloxy)cyclohexyl)-3-methyl-8-(1-methyl-1H-pyrazol-4-yl)-3H-pyrrolo[2,3-c]isoquinoline (440 mg, 0.976 mmol) in TFA (5 mL) was stirred at 90° C. for 2 hours. The reaction mixture was concentrated and the residue was dissolved in MeOH (5 mL) and treated with K₂CO₃ (1 g), then stirred for 1 hour and filtered. The filtrate was concentrated and the residue was purified by Combi Flash (0% to 100% EtOAc in PE) to give Int-17 (320 mg, yield: 90%) as a yellow solid.

### Preparation of 4-iodo-2,6-naphthyridin-3-amine (Int-18)

### Step 1: Preparation of tert-butyl 2-cyano-2-(4-cyanopyridin-3-yl)acetate

A mixture of 3-bromoisonicotinonitrile (9.00 g, 49.2 mmol), tert-butyl 2-cyanoacetate (8.33 g, 59.0 mmol), t-BuOK (11.6 g, 103 mmol) and Pd(dppf)Cl₂ (3.60 g, 4.92 mmol) in dioxane (100 mL) was degassed and purged with N₂ for 3 times, and then the mixture was stirred at 70° C. for 12 hours under N₂ atmosphere. The reaction mixture was concentrated and the residue was diluted with NH₄Cl (100 mL) and extracted with EtOAc (150 mL×3). The combined organic layers were washed with brine (150 mL), dried over anhydrous Na₂SO₄, filtered and concentrated. The residue was purified by silica gel column (0% to 30% EtOAc in PE) to give tert-butyl 2-cyano-2-(4-cyanopyridin-3-yl)acetate (6.68 g, yield: 56%) as yellow oil.

¹H NMR (400 MHz, CDCl₃) δ 1.53 (9H, s), 5.05 (1H, s), 7.65 (1H, d, J=4.8 Hz), 8.89 (1H, d, J=4.8 Hz), 9.02 (1H, s).

### Step 2: Preparation of 1-bromo-2,6-naphthyridin-3-amine

A mixture of tert-butyl 2-cyano-2-(4-cyanopyridin-3-yl)acetate (6.68 g, 27.5 mmol) in HBr (29.8 g, 121 mmol, 33% in HOAc) was stirred at 25° C. for 0.5 hour. The reaction mixture was basified with saturated aqueous NaHCO₃ to pH=7 and extracted with EtOAc (150 mL×3). The combined organic layers were washed with brine (150 mL), dried over anhydrous Na₂SO₄, filtered and concentrated. The residue was purified by silica gel column (0% to 100% EtOAc in PE) to give 1-bromo-2,6-naphthyridin-3-amine (1.40 g, yield: 23%) as a yellow solid.

¹H NMR (400 MHz, DMSO-d₆) δ 6.68 (2H, brs), 6.77 (1H, s), 7.61 (1H, d, J=6.0 Hz), 8.25 (1H, d, J=6.0 Hz), 9.05 (1H, s).

### Step 3: Preparation of 2,6-naphthyridin-3-amine

A mixture of 1-bromo-2,6-naphthyridin-3-amine (500 mg, 2.23 mmol), triethylsilane (311 mg, 2.68 mmol), XPhos-Pd-G₃ (189 mg, 0.223 mmol) and K₂CO₃ (617 mg, 4.46 mmol) in dioxane (8 mL) and H₂O (1 mL) was degassed and purged with N₂ for 3 times, and then the mixture was stirred at 90° C. for 16 hours under N₂ atmosphere. The reaction mixture was concentrated and the residue was purified by silica gel column (0% to 5% MeOH in DCM) to give 2,6-naphthyridin-3-amine (120 mg, yield: 37%) as a yellow solid.

¹H NMR (400 MHz, DMSO-d₆) δ 6.28 (2H, brs), 6.73 (1H, s), 7.66 (1H, d, J=5.6 Hz), 8.17 (1H, d, J=5.6 Hz), 8.93 (1H, s), 9.02 (1H, s).

### Step 4: Preparation of 4-iodo-2,6-naphthyridin-3-amine (Int-18)

To a solution of 2,6-naphthyridin-3-amine (120 mg, 0.827 mmol) in DMF (2 mL) was added NIS (186 mg, 0.827 mmol). The mixture was stirred at 25° C. for 1 hour. The reaction mixture was diluted with H₂O (10 mL) and extracted with EtOAc (20 mL×3). The combined organic layers were washed with brine (30 mL), dried over anhydrous Na₂SO₄, filtered and concentrated to give Int-18 (130 mg, yield: 50%) as a yellow solid.

¹H NMR (400 MHz, DMSO-d₆) δ 6.57 (2H, brs), 7.69 (1H, dd, J=5.6, 0.8 Hz), 8.36 (1H, d, J=5.2 Hz), 8.93 (1H, s), 9.02 (1H, s).

### Preparation of 4-iodo-2,7-naphthyridin-3-amine (Int-19)

### Step 1: Preparation of (E)-2,6-dichloro-4-(2-(dimethylamino)vinyl)nicotinonitrile

A mixture of 2,6-dichloro-4-methylnicotinonitrile (30.0 g, 160 mmol) in DMF (300 mL) was added DMF-DMA (38.2 g, 321 mmol). The mixture was stirred at 100° C. for 12 hours. The reaction mixture was concentrated and the residue was diluted with EtOAc (180 mL) and then poured into H₂O (600 mL) and stirred at 25° C. for 0.5 hour. The precipitate was filtered and washed with EtOAc (10 mL×2) and dried to give (E)-2,6-dichloro-4-(2-(dimethylamino)vinyl)nicotinonitrile (12.0 g, yield: 31%) as a gray solid.

¹H NMR (400 MHz, DMSO-d₆) δ 2.90 (3H, s), 3.10-3.24 (3H, m), 5.05 (1H, d, J=12.8 Hz), 7.60 (1H, s), 8.08 (1H, d, J=12.8 Hz).

### Step 2: Preparation of 6,8-dichloro-2,7-naphthyridin-1(2H)-one

A mixture of (E)-2,6-dichloro-4-(2-(dimethylamino)vinyl)nicotinonitrile (12.0 g, 49.6 mmol) in conc. HCl (64 mL) was stirred at 45° C. for 16 hours. After cooled down to room temperature, ice water (200 mL) was added to the reaction mixture. The precipitate was collected by filtration and dried to give 6,8-dichloro-2,7-naphthyridin-1(2H)-one (10.0 g, yield: 83%) as a yellow solid.

¹H NMR (400 MHz, DMSO-d₆) δ 6.53 (1H, dd, J=7.2, 1.2 Hz), 7.51 (1H, t, J=6.8 Hz), 7.78 (1H, s), 11.77 (1H, brs).

### Step 3: Preparation of 1,3,8-trichloro-2,7-naphthyridine

A solution of 6,8-dichloro-2,7-naphthyridin-1(2H)-one (2.00 g, 9.30 mmol) in POCl₃ (33.0 g, 215 mmol) was stirred at 160° C. for 2 hours. The reaction mixture was concentrated and the residue was basified with saturated aqueous NaHCO₃ to pH=8 at 0° C., then extracted with EtOAc (20 mL×2). The combined organic layer was dried anhydrous Na₂SO₄, filtered and concentrated. The residue was purified by silica gel column (PE/EtOAc=3/1) to afford 1,3,8-trichloro-2,7-naphthyridine (1.05 g, yield: 48%) as a gray solid.

¹H NMR (400 MHz, DMSO-d₆) δ 7.96 (1H, d, J=5.6 Hz), 8.28 (1H, s), 8.59 (1H, d, J=5.6 Hz).

### Step 4: Preparation of 3-chloro-2,7-naphthyridine

A solution of 1,3,8-trichloro-2,7-naphthyridine (1.05 g, 4.50 mmol), Pd(PPh₃)₄ (1.04 g, 0.899 mmol) and PPh₃ (354 mg, 1.35 mmol) in toluene (50 mL) was degassed and purged with N₂ for 3 times, and then n-Bu₃SnH (5.27 g, 18.1 mmol) was added and the resulting reaction mixture was stirred at 20° C. for 24 hours under N₂ atmosphere. The reaction mixture was concentrated and the residue was purified by silica gel column (PE/EtOAc=3/1) to afford 3-chloro-2,7-naphthyridine (240 mg, yield: 31%) as a gray solid.

### Step 5: Preparation of tert-butyl (2,7-naphthyridin-3-yl)carbamate

A mixture of 3-chloro-2,7-naphthyridine (172 mg, 1.05 mmol), BocNH₂ (367 mg, 3.14 mmol), Pd₂(dba)₃ (96 mg, 0.10 mmol), XPhos (100 mg, 0.209 mmol) and Cs₂CO₃ (681 mg, 2.09 mmol) in 1, 4-dioxane (5 mL) was degassed and purged with N₂ for 3 times, then the mixture was stirred at 90° C. for 12 hours under N₂ atmosphere. The reaction mixture was concentrated and the residue was purified by silica gel column (PE/EtOAc=3/1) to afford tert-butyl (2,7-naphthyridin-3-yl)carbamate (285 mg, yield: 88%) as a brown solid.

¹H NMR (400 MHz, DMSO-d₆) δ 1.51 (9H, s), 7.77 (1H, d, J=5.6 Hz), 8.15 (1H, s), 8.55 (1H, d, J=5.6 Hz), 9.28 (1H, s), 9.34 (1H, s), 10.21 (1H, brs).

### Step 6: Preparation of 2,7-naphthyridin-3-amine

A solution of tert-butyl (2,7-naphthyridin-3-yl)carbamate (285 mg, 1.16 mmol) in DCM (5 mL) and TFA (1.25 mL) was stirred at 20° C. for 1 hour. The reaction mixture was concentrated and the residue was basified with saturated aqueous NaHCO₃ to pH=8 and extracted with DCM (10 mL×3). The combined organic layers were dried over anhydrous Na₂SO₄, filtered and concentrated to give 2,7-naphthyridin-3-amine (123 mg, crude) as a yellow solid.

### Step 7: Preparation of 4-iodo-2,7-naphthyridin-3-amine (Int-19)

A solution of 2,7-naphthyridin-3-amine (123 mg, 0.847 mmol) in DMF (3 mL) was added NIS (191 mg, 0.847 mmol) and stirred at 0° C. for 3 hours. The reaction mixture was poured into water (5 mL) and the precipitate was filtered. The solid was washed with water (3 mL) and dried to give Int-19 (200 mg, yield: 87%) as a yellow solid.

### Preparation of tert-butyl 4-(prop-1-yn-1-yl)piperidine-1-carboxylate (Int-20)

A solution of tert-butyl 4-ethynylpiperidine-1-carboxylate (6.00 g, 28.7 mmol) in anhydrous THF (100 mL) was added n-BuLi (28 mL, 69.5 mmol, 2.5M in hexane) dropwise at −65° C. After the addition, the mixture was warmed up to 15° C. and MeI (10.2 g, 71.7 mmol) was added dropwise at 15° C. The resulting mixture was stirred at 15° C. for 12 hours. The reaction mixture was quenched by addition H₂O (100 mL) and extracted with EtOAc (150 mL×3). The combined organic layer was washed with brine (100 mL), dried over anhydrous Na₂SO₄, filtered and concentrated. The residue was purified by flash silica gel chromatography (PE/EtOAc=10/1) to give Int-20 (2.10 g, yield: 33%) as yellow oil.

¹H NMR (400 MHz, CDCl₃) δ 1.46 (9H, s), 1.49-1.57 (2H, m), 1.70-1.78 (2H, m), 1.81 (3H, s), 2.43-2.56 (1H, m), 3.05-3.22 (2H, m), 3.60-3.79 (2H, m).

### Preparation of tert-butyl 4-(3,3,3-trifluoroprop-1-yn-1-yl)piperidine-1-carboxylate (Int-21)

To a solution of 3,3-dimethyl-1-(trifluoromethyl)-1,3-dihydro-113-benzo[d][1,2]iodaoxole (2.84 g, 8.60 mmol), 1, 10-phenanthroline (413 mg, 2.29 mmol), CuI (218 mg, 1.15 mmol) and KHCO₃ (1.15 g, 11.5 mmol) in anhydrous DCM (20 mL) was added tert-butyl 4-ethynylpiperidine-1-carboxylate (1.20 g, 5.73 mmol) in anhydrous DCM (15 mL) dropwise over 2 hour at 25° C. under N₂ atmosphere and the reaction mixture was stirred at 45° C. for 16 hours under N₂ atmosphere. The reaction mixture turned into green suspension. The reaction mixture was diluted with H₂O (40 mL), DCM (20 mL) and separated. The aqueous phase was extracted with DCM (40 mL×3) and the combined organic layer was dried over anhydrous Na₂SO₄, filtered and concentrated. The residue was purified by flash silica gel chromatography (ISCO®; 20 g SepaFlash® Silica Flash Column, Eluent of 4˜6% Ethyl acetate/Petroleum ether gradient @40 mL/min) to give tert-butyl 4-(3,3,3-trifluoroprop-1-yn-1-yl)piperidine-1-carboxylate (1.40 g, yield: 88%) as colorless oil.

¹H NMR (400 MHz, CDCl₃) δ 1.47 (9H, s), 1.60-1.71 (2H, m), 1.79-1.90 (2H, m), 2.63-2.77 (1H, m), 3.12-3.24 (2H, m), 3.64-3.78 (2H, m).

### Preparation of 5-bromo-2-((tetrahydro-2H-pyran-4-yl)ethynyl)thiazole (Int-22)

### Step 1: Preparation of 5-bromo-2-iodothiazole

A mixture of 5-bromothiazol-2-amine (5.00 g, 27.9 mmol), isopentyl nitrite (4.91 g, 41.9 mmol), CH₂I₂ (8.98 g, 33.5 mmol) in THF (60 mL) was degassed and purged with N₂ for 3 times, then the mixture was stirred at 20° C. for 16 hours under N₂ atmosphere. The mixture turned into black solution. The reaction mixture was concentrated and the residue was purified by flash silica gel chromatography (ISCO®; 40 g SepaFlash® Silica Flash Column, Eluent of ˜2% Ethyl acetate/Petroleum ether gradient @40 mL/min) to give 5-bromo-2-iodothiazole (3.45 g, contains impurity) as brown oil.

### Step 2: Preparation of 5-bromo-2-((tetrahydro-2H-pyran-4-yl)ethynyl)thiazole (Int-22)

To a solution of impure 5-bromo-2-iodothiazole (2.64 g, contains impurity), Pd(PPh₃)₂Cl₂ (542 mg, 0.772 mmol), CuI (294 mg, 1.54 mmol) and TEA (3.90 g, 38.6 mmol) in THF (30 mL) was added 4-ethynyltetrahydro-2H-pyran (850 mg, 7.72 mmol) in THF (5 mL) dropwise under N₂ atmosphere and the reaction mixture was stirred at 35° C. for 3 hours under N₂ atmosphere to give brown solution. The reaction mixture was concentrated and the residue was purified by flash silica gel chromatography (ISCO®; 20 g SepaFlash® Silica Flash Column, Eluent of -10% Ethyl acetate/Petroleum ether gradient @45 mL/min) to give impure Int-22 (890 mg, contains impurity) as a yellow solid.

### Preparation of 8-(4,4,5,5-tetramethyl-1, 3,2-dioxaborolan-2-yl)-3H-pyrrolo[2,3-c]isoquinoline (Int-23)

### Step 1: Preparation of 6-bromo-4-iodoisoquinolin-3-amine

To a solution of 6-bromoisoquinolin-3-amine (3.00 g, 13.5 mmol) in anhydrous DMF (40 mL) was added NIS (3.33 g, 14.8 mmol) portion-wise at 0° C. and the mixture was stirred at 20° C. for 2 hours to give brown suspension. The reaction mixture was poured into a mixture of saturated aqueous Na₂CO₃ (100 mL) and ice water (100 mL). The precipitate was filtered and washed with H₂O (30 mL×2), then dried to give 6-bromo-4-iodoisoquinolin-3-amine (4.70 g, crude) as a brown solid, which was used into the next step without further purification.

### Step 2: Preparation of 6-bromo-4-((trimethylsilyl)ethynyl)isoquinolin-3-amine

A solution of 6-bromo-4-iodoisoquinolin-3-amine (4.70 g, 13.5 mmol), CuI (513 mg, 2.69 mmol), Pd(PPh₃)₂Cl₂ (945 mg, 1.35 mmol) and TEA (6.81 g, 67.3 mmol) in THF (80 mL) was degassed and purged with N₂ for 3 times, ethynyltrimethylsilane (1.59 g, 16.2 mmol) was added to the reaction mixture and the reaction mixture was stirred at 70° C. for 1.5 hours under N₂ atmosphere to give brown solution. The reaction mixture was concentrated and the residue was purified by flash silica gel chromatography (ISCO®; 40 g SepaFlash® Silica Flash Column, Eluent of ˜13% Ethyl acetate/Petroleum ether gradient @50 mL/min) to give 6-bromo-4-((trimethylsilyl)ethynyl)isoquinolin-3-amine (1.50 g, yield: 34% for two steps) as a yellow solid.

### Step 3: Preparation of 8-bromo-3H-pyrrolo[2,3-c]isoquinoline

To a solution of 6-bromo-4-((trimethylsilyl)ethynyl)isoquinolin-3-amine (300 mg, 0.940 mmol) in NMP (4 mL) was added NaH (207 mg, 5.17 mmol, 60% dispersion in mineral oil) at 0° C., the reaction mixture was stirred at 0° C. for 1 minute and stirred at 100° C. for 10 minutes to give brown solution. The reaction mixture was added dropwise into H₂O (80 mL) at 0° C., then extracted with EtOAc (70 mL×2). The combined organic layer was washed with brine (80 mL), dried over anhydrous Na₂SO₄ and concentrated. The residue was purified by flash silica gel chromatography (ISCO®; 12 g SepaFlash® Silica Flash Column, Eluent of ˜22% Ethyl acetate/Petroleum ether gradient @40 mL/min), then diluted with EtOAc (15 mL), washed with H₂O (15 mL×2), dried over anhydrous Na₂SO₄, filtered and concentrated to give 8-bromo-3H-pyrrolo[2,3-c]isoquinoline (115 mg, yield: 50%) as a white solid.

¹H NMR (400 MHz, DMSO-d₆) δ 7.11 (1H, dd, J=3.2, 2.0 Hz), 7.49 (1H, t, J=2.8 Hz), 7.62 (1H, dd, J=8.8, 2.0 Hz), 8.09 (1H, d, J=8.8 Hz), 8.55 (1H, d, J=1.6 Hz), 8.90 (1H, s), 12.09 (1H, brs).

### Step 4: Preparation of 8-(4,4,5,5-tetramethyl-1,3,2-dioxaborolan-2-yl)-3H-pyrrolo[2, 3-c]isoquinoline (Int-23)

To a solution of 8-bromo-3H-pyrrolo[2,3-c]isoquinoline (115 mg, 0.465 mmol) and Bis-Pin (295 mg, 1.16 mmol) in 1, 4-dioxane (4 mL) was added KOAc (137 mg, 1.40 mmol) and Pd(dppf)Cl₂ (51 mg, 0.070 mmol) under N₂ atmosphere, the mixture was stirred at 110° C. for 16 hours under N₂ atmosphere to give brown solution. The reaction mixture was filtered through a pad of celite and the solid was washed with 1, 4-dioxane (5 mL×3). The filtrate was concentrated to give Int-23 (300 mg, crude) as brown solid, which was used into the next step without further purification.

### Preparation of 1-methyl-8-(4,4,5,5-tetramethyl-1, 3,2-dioxaborolan-2-yl)-3H-pyrrolo[2, 3-c]isoquinoline (Int-24)

### Step 1: Preparation of 8-bromo-1-methyl-2-(trimethylsilyl)-3H-pyrrolo[2,3-c]isoquinoline

A mixture of Int-I (1.00 g, 2.87 mmol), Pd(dppf)Cl₂ (210 mg, 0.290 mmol), LiCl (121 mg, 2.87 mmol) and KOAc (562 mg, 5.73 mmol) in DMF (40 mL) was degassed and purged with N₂ for 3 times, then trimethyl(prop-1-yn-1-yl)silane (965 mg, 8.61 mmol) was added into the reaction mixture and stirred at 120° C. for 16 hours under N₂ atmosphere. The reaction mixture was filtered and the filtrate was concentrated. The residue was purified by flash silica gel chromatography (PE/EtOAc=1/1) to afford 8-bromo-1-methyl-2-(trimethylsilyl)-3H-pyrrolo[2,3-c]isoquinoline (370 mg, yield: 30%) as a yellow solid.

### Step 2: Preparation of 8-bromo-1-methyl-3H-pyrrolo[2,3-c]isoquinoline

A mixture of 8-bromo-1-methyl-2-(trimethylsilyl)-3H-pyrrolo[2,3-c]isoquinoline (370 mg, 1.11 mmol) in TFA (6 mL) was stirred 60° C. for 1 hour. The reaction mixture was concentrated and the residue was diluted with H₂O (30 mL), then basified with saturated aqueous Na₂CO₃ to pH=8 and extracted with EtOAc (30 mL×3). The combined organic layer was washed with brine (30 mL), dried over anhydrous Na₂SO₄, filtered and concentrated. The residue was purified by flash silica gel chromatography (PE/EtOAc=1/1) to afford 8-bromo-1-methyl-3H-pyrrolo[2,3-c]isoquinoline (100 mg, yield: 35%) as a yellow solid.

### Step 3: Preparation of 1-methyl-8-(4,4,5,5-tetramethyl-1, 3,2-dioxaborolan-2-yl)-3H-pyrrolo[2, 3-c]isoquinoline (Int-24)

A mixture of 8-bromo-1-methyl-3H-pyrrolo[2,3-c]isoquinoline (100 mg, 0.383 mmol), Bis-Pin (117 mg, 0.460 mmol), Pd(OAc)₂ (9 mg, 0.04 mmol), PCy₃ (22 mg, 0.077 mmol) and KOAc (75 mg, 0.77 mmol) in 1, 4-dioxane (5 mL) was degassed and purged with N₂ for 3 times, and then the mixture was stirred at 90° C. for 16 hours under N₂ atmosphere. The reaction mixture was concentrated and the residue was purified by flash silica gel chromatography (PE/EtOAc=1/1) to afford Int-24 (150 mg, crude) as a yellow solid.

### Preparation of 2-methyl-8-(4,4,5,5-tetramethyl-1, 3,2-dioxaborolan-2-yl)-3H-pyrrolo[2, 3-c]isoquinoline (Int-25)

### Step 1: Preparation of 6-bromo-4-(prop-1-yn-1-yl)isoquinolin-3-amine

A mixture of Int-I (500 mg, 1.43 mmol), Pd(PPh₃)₂Cl₂ (100 mg, 0.143 mmol), CuI (54 mg, 0.029 mmol) in Et₃N (10 mL) was degassed and purged with N₂ for 3 times at 0° C.

Propyne (4.30 mL, 4.30 mmol, 1 M in THF) was added into the reaction mixture and the mixture was stirred at 20° C. for 2 hours. The reaction mixture was concentrated and the residue was diluted with H₂O (50 mL), then extracted with EtOAc (60 mL×3). The combined organic layer was washed with brine (50 mL), dried over anhydrous Na₂SO₄, filtered and concentrated.

The residue was purified by flash silica gel column (PE/EtOAc=1/1) to afford 6-bromo-4-(prop-1-yn-1-yl)isoquinolin-3-amine (140 mg, yield: 37%) as a yellow solid.

### Step 2: Preparation of 8-bromo-2-methyl-3H-pyrrolo[2,3-c]isoquinoline

To a solution of 6-bromo-4-(prop-1-yn-1-yl)isoquinolin-3-amine (140 mg, 0.536 mmol) in DMF (5 mL) was added t-BuOK (240 mg, 2.14 mmol) at 20° C. Then the reaction mixture was stirred at 20° C. for 2 hours. The reaction mixture was concentrated and the residue was purified by flash silica gel column (PE/EtOAc=2/1) to afford 8-bromo-2-methyl-3H-pyrrolo[2,3-c]isoquinoline (120 mg, yield: 86%) as a yellow solid.

### Step 3: Preparation of 2-methyl-8-(4,4,5,5-tetramethyl-1, 3,2-dioxaborolan-2-yl)-3H-pyrrolo[2, 3-c]isoquinoline (Int-25)

A mixture of 8-bromo-2-methyl-3H-pyrrolo[2,3-c]isoquinoline (120 mg, 0.459 mmol), Bis-Pin (140.04 mg, 0.551 mmol), Pd(OAc)₂ (10 mg, 0.046 mmol), PCy₃ (26 mg, 0.091 mmol) and KOAc (90 mg, 0.92 mmol) in 1, 4-dioxane (3 mL) was degassed and purged with N₂ for 3 times, then the mixture was stirred at 90° C. for 16 hours under N₂ atmosphere. The reaction mixture was concentrated and the residue was purified by flash silica gel column (PE/EtOAc =2/1) to afford Int-25 (140 mg, yield: 98%) as a yellow solid.

¹H NMR (400 MHz, DMSO-d₆) δ 1.36 (12H, s), 2.48 (3H, s), 6.78 (1H, s), 7.67 (1H, dd, J=8.0, 0.4 Hz), 8.05 (1H, d, J=8.0 Hz), 8.48 (1H, s), 8.79 (1H, s), 11.86 (1H, brs).

### Preparation of (cyclobutylethynyl)trimethylsilane (Int-26)

### Step 1: Preparation of (6-chlorohex-1-yn-1-yl)trimethylsilane

To a solution of ethynyltrimethylsilane (10.0 g, 102 mmol) in anhydrous THF (80 mL) was added n-BuLi (40.7 mL, 102 mmol, 2.5 M in hexane) dropwise at 0° C. under N₂ atmosphere. The reaction mixture was stirred at 0° C. for 0.5 hour. A solution of 1-chloro-4-iodo-butane (22.2 g, 102 mmol) in anhydrous THF (60 mL) was added dropwise at 0° C. The reaction mixture was allowed to warm to 20° C. and stirred for 15.5 hours to give light yellow solution. The mixture was quenched with ice-water (400 mL) and concentrated. The aqueous phase was extracted with EtOAc (300 mL×2). The extracts were washed with brine (300 mL), dried over anhydrous Na₂SO₄, filtered and concentrated. The residue was purified by flash silica gel chromatography (ISCO®; 120 g SepaFlash® Silica Flash Column, Eluent of 1-15% Ethyl acetate/Petroleum ether gradient @100 mL/min) to give (6-chlorohex-1-yn-1-yl)trimethylsilane (17.4 g, yield: 91%) as colorless oil.

### Step 2: Preparation of (cyclobutylethynyl)trimethylsilane (Int-26)

To a solution of i-Pr₂NH (18.7 g, 184 mmol) in THF (400 mL) was added n-BuLi (81.1 mL, 203 mmol, 2.5M in hexane) dropwise at 0° C. under N₂ atmosphere, the mixture was stirred at 0° C. for 0.5 hour. The reaction mixture was cooled to −70° C., (6-chlorohex-1-yn-1-yl)trimethylsilane (17.4 g, 92.2 mmol) in THF (60 mL) was added to the reaction mixture dropwise and the reaction mixture was stirred at −70° C. for 1 hour and stirred at 20° C. for 16 hours under N₂ atmosphere to give yellow solution. The reaction mixture was quenched with saturated aqueous NH₄Cl (300 mL) carefully at 0° C. and separated, the aqueous phase was extracted with pentane (200 mL×2). The combined organic layer was washed with brine (300 mL), dried over anhydrous Na₂SO₄, filtered and concentrated (bath temp. <10° C.). The residue was distilled under reduced pressure to collect the distillation at 76-80° C./˜100 kPa to give Int-26 (4.20 g, yield: 30%) as yellow oil.

¹H NMR (400 MHz, CDCl₃) δ 0.16 (9H, s), 1.81-1.96 (2H, m), 2.08-2.20 (2H, m), 2.21-2.33 (2H, m), 2.99-3.10 (1H, m).

### Preparation of (5-(3-((tert-butyldimethylsilyl)oxy)pyrrolidin-1-yl)pyridin-3-yl)boronic acid (Int-27)

### Step 1: Preparation of 3-((tert-butyldimethylsilyl)oxy)pyrrolidine

To a solution of pyrrolidin-3-ol (1.00 g, 11.5 mmol) and TBSCl (2.60 g, 17.2 mmol) in MeCN (20 mL) was added imidazole (2.34 g, 34.4 mmol) at 0° C., the mixture was stirred at 20° C. for 16 hours under N₂ atmosphere to give colorless solution. The reaction mixture was concentrated and the residue was diluted with EtOAc (50 mL), washed with saturated aqueous NaHCO₃ (50 mL), H₂O (50 mL), brine (50 mL), dried over anhydrous Na₂SO₄, filtered and concentrated to give 3-((tert-butyldimethylsilyl)oxy)pyrrolidine (2.30 g, crude) as pale oil, which was used into the next step without further purification.

### Step 2: Preparation of 3-bromo-5-(3-((tert-butyldimethylsilyl)oxy)pyrrolidin-1-yl)pyridine

To a solution of 3-bromo-5-iodopyridine (2.90 g, 10.2 mmol) and 3-((tert-butyldimethylsilyl)oxy)pyrrolidine (2.06 g, 10.2 mmol) in 1, 4-dioxane (40 mL) was added Xantphos (1.18 g, 2.04 mmol), Pd₂(dba)₃ (935 mg, 1.02 mmol) and Cs₂CO₃ (6.66 g, 20.4 mmol) under N₂ atmosphere, the mixture was stirred at 100° C. for 12 hours under N₂ atmosphere to give brown solution. The reaction mixture was concentrated and the residue was diluted with H₂O (50 mL), extracted with EtOAc (50 mL×2). The combined organic layer was washed with brine (60 mL), dried over anhydrous Na₂SO₄, filtered and concentrated. The residue was purified by flash silica gel chromatography (ISCO®; 20 g SepaFlash® Silica Flash Column, Eluent of 9˜12% Ethyl acetate/Petroleum ether gradient @40 mL/min) to give 3-bromo-5-(3-((tert-butyldimethylsilyl)oxy)pyrrolidin-1-yl)pyridine (2.60 g, yield: 71%) as yellow oil.

¹H NMR (400 MHz, CDCl₃) δ 0.10 (6H, d, J=4.4 Hz), 0.89 (9H s), 1.97-2.04 (1H, m), 2.07-2.16 (1H, m), 3.10-3.20 (1H, m), 3.30-3.38 (1H, m), 3.40-3.54 (2H, m), 4.52-4.62 (1H, m), 6.93 (1H, s), 7.81-8.09 (2H, m).

### Step 3: Preparation of (5-(3-((tert-butyldimethylsilyl)oxy)pyrrolidin-1-yl)pyridin-3-yl)boronic acid (Int-27)

To a solution of 3-bromo-5-(3-((tert-butyldimethylsilyl)oxy)pyrrolidin-1-yl)pyridine (1.30 g, 3.64 mmol) and Bis-Pin (1.39 g, 5.46 mmol) in 1, 4-dioxane (20 mL) was added KOAc (714 mg, 7.28 mmol) and Pd(dppf)Cl₂ (266 mg, 0.364 mmol) under N₂ atmosphere, the mixture was stirred at 110° C. for 16 hours under N₂ atmosphere to give brown solution. The reaction mixture was filtered through a pad of celite and the solid was washed with 1, 4-dioxane (10 mL×2). The filtrate was concentrated to give Int-27 (2.80 g, crude) as black brown gum, which was used for the next step without further purification.

### Preparation of 4-iodo-7-(1-methyl-1H-pyrazol-4-yl)isoquinolin-3-amine (Int-28)

### Step 1: Preparation of 7-(1-methyl-1H-pyrazol-4-yl)isoquinolin-3-amine

A mixture of 7-bromoisoquinolin-3-amine (7.20 g, 32.3 mmol), 1-methyl-4-(4,4,5,5-tetramethyl-1,3,2-dioxaborolan-2-yl)-1H-pyrazole (10.1 g, 48.4 mmol), Pd(dppf)Cl₂ (2.36 g, 3.23 mmol) and Na₂CO₃ (6.84 g, 64.6 mmol) in 1,4-dioxane (70 mL) and H₂O (7 mL) was degassed and purged with N₂ for 3 times, then the mixture was stirred at 90° C. for 12 hours under N₂ atmosphere. The reaction mixture was quenched with water (50 mL) and extracted with EtOAc (100 mL×2). The combined organic layer was dried over anhydrous Na₂SO₄, filtered and concentrated. The residue was triturated with MeOH (30 ml) to give 7-(1-methyl-1H-pyrazol-4-yl)isoquinolin-3-amine (5.60 g, yield: 77%) as a gray solid.

### Step 2: Preparation of 4-iodo-7-(1-methyl-1H-pyrazol-4-yl)isoquinolin-3-amine (Int-28)

To a solution of 7-(1-methyl-1H-pyrazol-4-yl)isoquinolin-3-amine (1.00 g, 4.50 mmol) in DMF (20 mL) was added NIS (1.10 g, 4.90 mmol). The mixture was stirred at 25° C. for 2 hours. The reaction mixture was concentrated under reduced pressure. The residue was purified by Combi Flash (0% to 100% EtOAc in PE) to give Int-28 (300 mg, yield: 19%) as brown gum.

### Preparation of Compounds of Formula (I)

### Example 1. Preparation of Compound 1

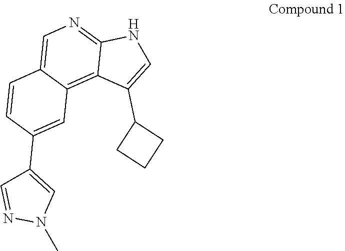 [SMILES: Cn1cc(-c2ccc3cnc4[nH]cc(C5CCC5)c4c3c2)cn1]

### Step 1: Preparation of 8-bromo-1-cyclobutyl-2-(trimethylsilyl)-3H-pyrrolo[2,3-c]isoquinoline

A mixture of Int-1 (400 mg, 1.15 mmol), Int-26 (210 mg, 1.38 mmol), LiCl (49 mg, 1.2 mmol), Na₂CO₃ (36 mg, 3.44 mmol) and Pd(dppf)Cl₂ (84 mg, 0.12 mmol) in anhydrous DMF (10 mL) was degassed and purged with N₂ for 3 times. Then the reaction mixture was stirred at 100° C. under N₂ atmosphere for 12 hours to give a yellow mixture. The reaction mixture was poured into brine (40 mL) and extracted with EtOAc (30 mL×5). The combined organic layer was washed with brine (50 mL×2), dried over Na₂SO₄ and concentrated. The residue was purified by Combi Flash (SiO₂, EtOAc 0% to 25% in PE) to give 8-bromo-1-cyclobutyl-2-(trimethylsilyl)-3H-pyrrolo[2,3-c]isoquinoline (170 mg, yield: 40%) as a yellow solid.

### Step 2: Preparation of 8-bromo-1-cyclobutyl-3H-pyrrolo[2,3-c]isoquinoline

To a solution of 8-bromo-1-cyclobutyl-2-(trimethylsilyl)-3H-pyrrolo[2,3-c]isoquinoline (170 mg, 0.455 mmol) in THF (6 mL) was added TBAF (0.91 mL, 0.91 mmol, 1 M in THF) at 0° C. The reaction mixture was stirred at 25° C. for 2 hour to give a yellow mixture. The reaction mixture was poured into saturated NH₄Cl (20 mL) and extracted with EtOAc (20 mL×3). The combined organic layer was washed with saturated NH₄Cl (20 mL×5), dried over anhydrous Na₂SO₄, filtered and concentrated to give 8-bromo-1-cyclobutyl-3H-pyrrolo[2, 3-c]isoquinoline (100 mg, yield: 73%) as a yellow solid.

¹H NMR (400 MHz, CDCl₃) δ 1.85-1.95 (2H, m), 2.05-2.15 (2H, m), 2.45-2.55 (2H, m), 3.88-3.98 (1H, m), 7.09 (1H, s), 7.45 (1H, dd, J=8.4, 1.6 Hz), 7.78 (1H, d, J=8.8 Hz), 8.25 (1H, s), 8.72 (1H, s), 9.07 (1H, brs).

### Step 3: Preparation of 1-cyclobutyl-8-(1-methyl-1H-pyrazol-4-yl)-3H-pyrrolo[2,3-c]isoquinoline (Compound 1)

A mixture of 8-bromo-8-cyclobutyl-3H-pyrrolo[2,3-c]isoquinoline (100 mg, 0.332 mmol), (1-methyl-pH-pyrazol-4-yl)boronic acid (84 mg, 0.66 mmol) Pd(dppf)Cl₂ (27 mg, 0.033 mmol) and Na₂CO₃ (106 mg, 0.996 mmol) in dioxane (3 mL) and H₂J (0.4 mL) was degassed and purged with N₂ for 3 times. Then the resulting reaction mixture was stirred at 90° C. for 6 hours to give a yellow mixture. The reaction mixture was poured into water (5 mL) and extracted with EtOAc (10 mL×3). The combined organic layer was dried over anhydrous Na₂SO₄, filtered and concentrated. The residue was purified by Combi Flash (SiO₂, EtOAc 000 to 80% in PE) to give Compound 1(60 mg, yield: 60H) as a8 yellow solid.

¹H NMR (400 MHz, DMSO-d₆) δ 1.90-2.00 (1H, m), 2.15-2.25 (3H, i), 2.55-2.65 (2H, i), 3.95 (3H, s), 4.10-4.20 (1H, n), 7.31 (1H, d, J=2.0 Hz), 7.71 (1H, dd, J 8.4, 1.6 Hz), 8.04 (1H, s), 8.10 (1H, d, J=8.4 Hz), 8.22 (1H, s), 8.35 (1H, s), 8.79 (1H, s), 11.73 (1H, brs),

The following compounds were synthesized analogously to Compound 1

| Comp. | | | |
|---|---|---|---|
| No | Structure | Name | ¹H NMR (400 MHz) |
| 2 | 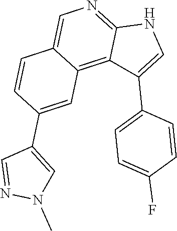 [SMILES: Cn1cc(-c2ccc3cnc4[nH]cc(-c5ccc(F)cc5)c4c3c2)cn1] | 1-(4-fluorophenyl)- 8-(1-methyl-1H- pyrazol-4-yl)-3H- pyrrolo[2,3- c]isoquinoline | CDCl₃; δ 3.88 (3H, s), 7.10-7.20 (2H, m), 7.46 (1H, s), 7.51 (1H, dd, J = 8.4, 1.2 Hz), 7.54-7.61 (3H, m), 7.95 (1H, d, J = 8.4 Hz), 8.09 (1H, s), 8.80 (1H, s), 9.23 (1H, s). |
| 3 | 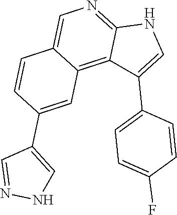 [SMILES: Fc1ccc(-c2c[nH]c3ncc4ccc(-c5cn[nH]c5)cc4c23)cc1] | 1-(4-fluorophenyl)- 8-(1H-pyrazol-4-yl)- 3H-pyrrolo[2,3- c]isoquinoline | DMSO-d₆; δ 7.40-7.50 (3H, m), 7.60-7.70 (3H, m), 7.75 (1H, d, J = 8.4 Hz), 8.05-8.15 (3H, m), 8.88 (1H, s), 12.19 (1H, brs), 13.03- 13.13 (1H, m). |
| 6 | 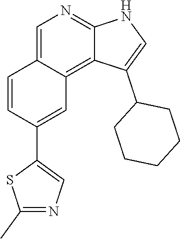 [SMILES: Cc1ncc(-c2ccc3cnc4[nH]cc(C5CCCCC5)c4c3c2)s1] | 5-(1-cyclohexyl-3H- pyrrolo[2,3- c]isoquinolin-8-yl)- 2-methylthiazole | DMSO-d₆; δ 1.30-1.40 (1H, m), 1.45-1.50 (2H, m), 1.55-1.70 (2H, m), 1.80-1.90 (3H, m), 2.10-2.20 (2H, m), 2.75 (3H, s), 3.10-3.20 (1H, m), 7.24 (1H, d, J = 2.4 Hz), 7.87 (1H, d, J = 8.8 Hz), 8.19 (1H, d, J = 8.8 Hz), 8.20-8.30 (2H, m), 8.86 (1H, s), 11.84 (1H, brs). |
| 65 | 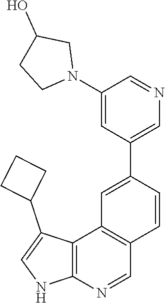 [SMILES: OC1CCN(c2cncc(-c3ccc4cnc5[nH]cc(C6CCC6)c5c4c3)c2)C1] | 1-(5-(1-cyclobutyl- 3H-pyrrolo[2,3- c]isoquinolin-8- yl)pyridin-3- yl)pyrrolidin-3-ol | — |
| 66* | 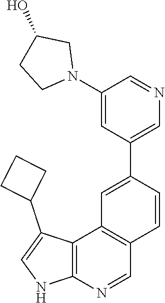 [SMILES: O[C@H]1CCN(c2cncc(-c3ccc4cnc5[nH]cc(C6CCC6)c5c4c3)c2)C1] | (S)-1-(5-(1- cyclobutyl-3H- pyrrolo[2,3- c]isoquinolin-8- yl)pyridin-3- yl)pyrrolidin-3-ol | DMSO-d₆; δ 1.90-2.01 (2H, m), 2.08-2.28 (4H, m), 2.54-2.61 (2H, m), 3.20-3.27 (1H, m), 3.40-3.52 (2H, m), 3.52-3.58 (1H, m), 4.09- 4.20 (1H, m), 4.43-4.50 (1H, m), 5.07 (1H, brs), 7.19 (1H, t, J = 2.4 Hz), 7.36 (1H, s), 7.83 (1H, dd, J = 8.8, 2.0 Hz), 8.01 (1H, d, J = 2.8 Hz), 8.23 (1H, d, J = 8.4 Hz), 8.28 (1H, d, J = 1.6 Hz), 8.37 (1H, s), 8.91 (1H, s), 11.85 (1H, brs). |
| 67* | 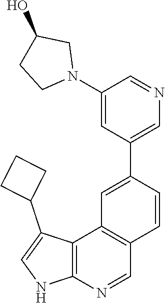 [SMILES: O[C@@H]1CCN(c2cncc(-c3ccc4cnc5[nH]cc(C6CCC6)c5c4c3)c2)C1] | (R)-1-(5-(1- cyclobutyl-3H- pyrrolo[2,3- c]isoquinolin-8- yl)pyridin-3- yl)pyrrolidin-3-ol | DMSO-d₆; δ 1.89-2.01 (2H, m), 2.09-2.28 (4H, m), 2.53-2.60 (2H, m), 3.20-3.27 (1H, m), 3.40-3.51 (2H, m), 3.52-3.58 (1H, m), 4.08- 4.20 (1H, m), 4.43-4.50 (1H, m), 5.05 (1H, brs), 7.17-7.22 (1H, m), 7.36 (1H, s), 7.83 (1H, dd, J = 8.8, 1.6 Hz), 8.01 (1H, d, J = 2.0 Hz), 8.23 (1H, d, J = 8.4 Hz), 8.28 (1H, s), 8.37 (1H, s), 8.91 (1H, s), 11.85 (1H, brs). |

*SFC separation (column: DAICEL CHIRALPAK AD (250 mm* 30 mm, 10 um); mobile phase: [0.1% NH₃H₂O IPA]; B%: 50%-50%)

### Example 2. Preparation of Compound 4

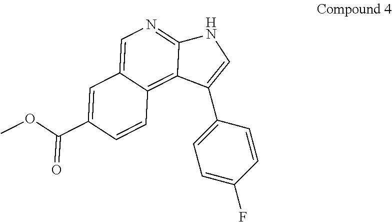 [SMILES: COC(=O)c1ccc2c(cnc3[nH]cc(-c4ccc(F)cc4)c32)c1]

### Step 1: Preparation of methyl 3-aminoisoquinoline-7-carboxylate

A mixture of 7-bromoisoquinolin-3-amine (1.50 g, 6.72 mmol), Pd(dppf)Cl₂·CH₂Cl₂ (549 mg, 0.672 mmol), TEA (2.04 g, 20.2 mmol) in MeOH (30 mL) and DMF (6 mL) was degassed and purged with CO for 3 times, and then the mixture was stirred at 60° C. for 43 hours under CO atmosphere (50 psi). The reaction mixture was filtered by a pad of celite and the filtrate was concentrated. The residue was purified by Combi Flash (0% to 50% EtOAc in PE) to give methyl 3-aminoisoquinoline-7-carboxylate (670 mg, yield: 44%) as a light red solid.

¹H NMR (400 MHz, CDCl₃) δ 3.97 (3H, s), 4.67 (2H, brs), 6.73 (1H, s), 7.54 (1H, d, J=8.8 Hz), 8.06 (1H, dd, J=8.8 Hz), 8.55 (1H, s), 8.96 (1H, s).

### Step 2: Preparation of methyl 3-amino-4-iodoisoquinoline-7-carboxylate

To a solution of methyl 3-aminoisoquinoline-7-carboxylate (100 mg, 0.495 mmol) in DMF (2 mL) was added NIS (100 mg, 0.445 mmol) at 0° C. The mixture was stirred at 0° C. for 1 hour. The reaction mixture was poured into ice-water (10 mL) and the precipitate was filtered and dried under vacuum to give methyl 3-amino-4-iodoisoquinoline-7-carboxylate (120 mg, yield: 56%) as a light red solid.

### Step 3: Preparation of methyl 1-(4-fluorophenyl)-2-(trimethylsilyl)-3H-pyrrolo[2,3-c]isoquinoline-7-carboxylate

A mixture of compound methyl 3-amino-4-iodoisoquinoline-7-carboxylate (60 mg, 0.18 mmol), ((4-fluorophenyl)ethynyl)trimethylsilane (63 mg, 0.33 mmol), Pd(dppf)Cl₂ (13 mg, 0.018 mmol), LiCl (8 mg, 0.02 mmol) and K₂CO₃ (76 mg, 0.55 mmol) in DMF (5 mL) was degassed and purged with N₂ for 3 times and then the mixture was stirred at 110° C. for 1 hour under N₂ atmosphere. The reaction mixture was filtered by a pad of celite and the solid was washed with EtOAc (5 mL×2). The filtrate was concentrated and the residue was purified by Combi Flash (0% to 50% EtOAc in PE) to give methyl 1-(4-fluorophenyl)-2-(trimethylsilyl)-3H-pyrrolo[2,3-c]isoquinoline-7-carboxylate (30 mg, yield: 41%) as a light brown solid.

¹H NMR (400 MHz, CDCl₃) δ 0.20 (9H, s), 3.97 (3H, s), 7.15-7.25 (2H, m), 7.42-7.49 (2H, m), 7.61 (1H, d, J=8.8 Hz), 8.06 (1H, dd, J=8.8, 1.6 Hz), 8.78 (1H, s), 9.00 (1H, s), 9.31 (1H, brs).

### Step 4: Preparation of methyl 1-(4-fluorophenyl)-3H-pyrrolo[2,3-c]isoquinoline-7-carboxylate (Compound 4)

To a solution of methyl 1-(4-fluorophenyl)-2-(trimethylsilyl)-3H-pyrrolo[2,3-c]isoquinoline-7-carboxylate (30 mg, 0.076 mmol) in THF (1 mL) was added TBAF (0.15 mL, 0.15 mmol, 1M in THF) at 20° C. The mixture was stirred at 20° C. for 1 hour. The reaction mixture was diluted with 1N aqueous HCl (3 mL) and extracted with EtOAc (5 mL×2). The combined organic layer was washed with 1N aqueous HCl (5 mL×2) and brine (6 mL), dried over anhydrous Na₂SO₄ and concentrated. The residue was purified by prep-HPLC (0.2% FA as an additive), then lyophilized to give Compound 4 (7.04 mg, yield: 28%) as an off-white solid.

¹H NMR (400 MHz, CDCl₃) δ 4.00 (3H, s), 7.15-7.25 (2H, m), 7.34 (1H, d, J=2.4 Hz), 7.55-7.60 (2H, m), 8.08 (1H, d, J=8.8 Hz), 8.14 (1H, d, J=1.6 Hz), 8.82 (1H, d, J=2.0 Hz), 9.04 (1H, s), 9.64 (1H, brs).

### Example 3. Preparation of Compound 5

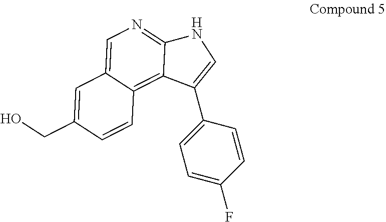 [SMILES: OCc1ccc2c(cnc3[nH]cc(-c4ccc(F)cc4)c32)c1]

To a solution of Compound 4 (240 mg, 0.749 mmol) in THF (5 mL) was added DIBAL-H (4.5 mL, 4.5 mmol, 1M in toluene) at −65° C. The mixture was stirred at −65° C. for 2 hours. The reaction mixture was quenched with saturated aqueous NH₄Cl (5 mL) dropwise at −65° C., then diluted with H₂O (3 mL) and extracted with EtOAc (6 mL×3). The combined organic layer was washed with brine (10 mL), dried over anhydrous Na₂SO₄, filtered and concentrated. The residue was purified by Combi Flash (0% to 50% EtOAc in PE) to give Compound 5 (19.06 mg, yield: 8%) as a yellow solid.

¹H NMR (400 MHz, DMSO-d₆) δ 4.66 (2H, d, J=5.6 Hz), 5.33 (1H, t, J=5.6 Hz), 7.30-7.40 (2H, m), 7.47 (1H, s), 7.50-7.55 (1H, m), 7.60-7.65 (2H, m), 7.95 (1H, d, J=8.4 Hz), 8.06 (1H, s), 8.93 (1H, s), 12.20 (1H, brs).

### Example 4. Preparation of Compound 7

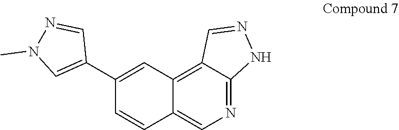 [SMILES: Cn1cc(-c2ccc3cnc4[nH]ncc4c3c2)cn1]

### Step 1: Preparation of (Z)-3-hydroxy-2-(3-methoxyphenyl)acrylonitrile

To a mixture of 2-(3-methoxyphenyl)acetonitrile (23.0 g, 156 mmol) and ethyl formate (12.2 g, 164 mmol) in anhydrous THF (1800 mL) was added dropwise a suspension of NaH (6.88 g, 172 mmol, 60% dispersion in mineral oil) in anhydrous THF (200 mL). The reaction mixture was stirred at 50° C. for 5 hours. The mixture was carefully acidified with 1M aqueous HCl to pH=2 and concentrated. The residue was poured into H₂O (500 mL) and extracted with EtOAc (500 mL×3). The combined organic layer was washed with brine (800 mL), dried over anhydrous Na₂SO₄, filtered and concentrated to give (Z)-3-hydroxy-2-(3-methoxyphenyl)acrylonitrile(29.0 g, crude) as a brown solid, which was directly used in the next step without purification.

¹H NMR (400 MHz, DMSO-d₆) δ 3.75 (1.2H, s), 3.76 (1.8H, s), 6.78-6.86 (1H, m), 6.93-7.00 (1.2H, m), 7.18-7.34 (1.8H, m), 7.65 (0.4H, s), 8.06 (0.6H, s), 12.12 (1H, brs).

### Step 2: Preparation of 1-benzyl-4-(3-methoxyphenyl)-1H-pyrazol-5-amine

To a solution of (Z)-3-hydroxy-2-(3-methoxyphenyl)acrylonitrile (29.0 g, crude) in EtOH (500 mL) was added benzylhydrazine hydrochloride (52.5 g, 331 mmol) and AcOH (24.9 g, 414 mmol), then the mixture was stirred at 85° C. for 3 hours. The reaction mixture was concentrated and the residue was basified with saturated aqueous NaHCO₃ to pH=8. Then the mixture was diluted with H₂O (600 mL) and extracted with EtOAc (600 mL×2). The combined organic layer was washed with brine (800 mL), dried over anhydrous Na₂SO₄ and concentrated. The residue was purified by Combi Flash (ISCO®; 120 g SepaFlash® Silica Flash Column, Eluent of 0˜2% methanol/dichloromethane gradient @80 mL/min) to give 1-benzyl-4-(3-methoxyphenyl)-1H-pyrazol-5-amine (33.0 g, yield: 74% for 2 steps) as a yellow solid.

### Step 3: Preparation of 3-benzyl-8-methoxy-3H-pyrazolo[3,4-c]isoquinoline

A solution of 1-benzyl-4-(3-methoxyphenyl)-1H-pyrazol-5-amine (1.00 g, 3.58 mmol) and paraformaldehyde (107 mg, 3.58 mmol) in TFA (25 mL) was stirred at 23° C. for 22 hours. Then another batch of paraformaldehyde (107 mg, 3.58 mmol) was added to the mixture and the mixture was stirred at 23° C. for 18 hours. We did this reaction for 9 batches in parallel. The reaction mixtures were combined and concentrated. The residue was basified with 2M aqueous NaOH to pH=11 and diluted with H₂O (80 mL), then extracted with DCM (70 mL×3). The combined organic layer was washed with brine (100 mL), dried over anhydrous Na₂SO₄, filtered and concentrated. The residue was purified by Combi Flash (ISCO®; 80 g SepaFlash® Silica Flash Column, Eluent of 0˜16% Ethyl acetate/Petroleum ether gradient @50 m/min) to give 3-benzyl-8-methoxy-3H-pyrazolo[3,4-c]isoquinoline (1.35 g, yield: 13%) as a white solid.

¹H NMR (400 MHz, CDCl₃) δ 4.02 (3H, s), 5.81 (2H, s), 7.17 (1H, dd, J=8.8, 2.4 Hz), 7.22-7.27 (1H, m), 7.30-7.40 (4H, m), 7.46 (1H, d, J=2.4 Hz), 7.97 (1H, d, J=8.8 Hz), 8.36 (1H, s), 8.94 (1H, s).

### Step 4: Preparation of 3H-pyrazolo[3,4-c]isoquinolin-8-ol

A solution of 3-benzyl-8-methoxy-3H-pyrazolo[3,4-c]isoquinoline (1.35 g, 4.67 mmol) in 55% aqueous HI (50 mL) was stirred at 100° C. for 12 hours. The reaction mixture was concentrated and the residue was poured into H₂O (60 mL), then basified with saturated aqueous NaHCO₃ to pH=8 and extracted with EtOAc (60 mL×3). The combined organic layer was dried over anhydrous Na₂SO₄, filtered and concentrated. The residue was purified by Combi Flash (ISCO®; 40 g SepaFlash® Silica Flash Column, Eluent of 0˜70% Ethyl acetate/Petroleum ether gradient @40 mL/min) and triturated with MeCN (6 mL) for twice and triturated with DCM (6 mL) for twice to give 3H-pyrazolo[3,4-c]isoquinolin-8-ol (540 mg, yield: 50%, LCMS purity: 80%) as an-off white solid.

### Step 5: Preparation of 3H-pyrazolo[3,4-c]isoquinolin-8-yl trifluoromethanesulfonate

To a solution of 3H-pyrazolo[3,4-c]isoquinolin-8-ol (130 mg, 0.562 mmol, purity: 80%) and PhNTf₂ (301 mg, 0.842 mmol) in DMF (4 mL) was added DIEA (436 mg, 3.37 mmol), the mixture was stirred at 20° C. for 3 hours. The reaction mixture was poured into water (30 mL) and extracted with EtOAc (25 mL×2). The combined organic layer was washed with brine (30 mL), dried over anhydrous Na₂SO₄, filtered and concentrated. The residue was purified by Combi Flash (ISCO®; 4 g SepaFlash® Silica Flash Column, Eluent of 0˜33% Ethyl acetate/Petroleum ether gradient @20 mL/min) to give 3H-pyrazolo[3,4-c]isoquinolin-8-yl trifluoromethanesulfonate (200 mg, yield: 96%) as a white solid.

### Step 6: Preparation of 8-(1-methyl-1H-pyrazol-4-yl)-3H-pyrazolo[3,4-c]isoquinoline (Compound 7)

To a solution of 3H-pyrazolo[3,4-c]isoquinolin-8-yl trifluoromethanesulfonate (200 mg, 0.541 mmol) and (1-methyl-1H-pyrazol-4-yl)boronic acid (89 mg, 0.70 mmol) in 1, 4-dioxane (4 mL) and H₂O (1 mL) was added Pd(dppf)Cl₂ (40 mg, 0.054 mmol) and Na₂CO₃ (115 mg, 1.08 mmol) under N₂ atmosphere. Then the mixture was stirred at 90° C. for 12 hours under N₂ atmosphere. The reaction mixture was poured into water (30 mL) and extracted with EtOAc (30 mL×3). The combined organic layer was washed with brine (40 mL), dried over anhydrous Na₂SO₄, filtered and concentrated. The residue was purified by flash silica gel chromatography (ISCO®; 4 g SepaFlash® Silica Flash Column, Eluent of 0˜75% Ethyl acetate/Petroleum ether gradient @20 mL/min) to give Compound 7 (53 mg, yield: 39%) as a white solid.

¹H NMR (400 MHz, CD₃OD) δ 3.99 (3H, s), 7.83 (1H, d, J=8.4 Hz), 8.10 (1H, s), 8.15 (1H, d, J=8.0 Hz), 8.24 (1H, s), 8.49 (1H, s), 8.57 (1H, s), 9.02 (1H, s).

### Example 5. Preparation of Compound 8

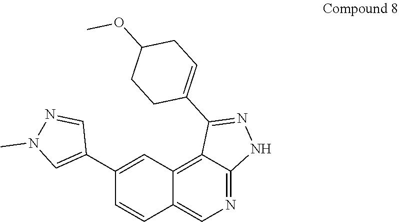 [SMILES: COC1CC=C(c2n[nH]c3ncc4ccc(-c5cnn(C)c5)cc4c23)CC1]

### Step 1: Preparation of 1-iodo-8-(1-methyl-1H-pyrazol-4-yl)-3H-pyrazolo[3,4-c]isoquinoline

To a mixture of Compound 7 (40 mg, 0.16 mmol) in THF (1 mL) was added t-BuOK (40 mg, 0.35 mmol) and 12 (86 mg, 0.34 mmol) in one portion at 20° C., the mixture was stirred at 20° C. for 12 hours. The reaction mixture was quenched with saturated aqueous Na₂SO₃ (10 mL), then poured into H₂O (15 mL) and extracted with EtOAc (15 mL×3). The combined organic layer was washed with brine (20 mL), dried over anhydrous Na₂SO₄, filtered and concentrated. The residue was purified by flash silica gel chromatography (ISCO®; 4 g SepaFlash® Silica Flash Column, Eluent of 0˜65% Ethyl acetate/Petroleum ether gradient @20 mL/min) to give 1-iodo-8-(1-methyl-1H-pyrazol-4-yl)-3H-pyrazolo[3,4-c]isoquinoline (35 mg, yield: 48%) as a yellow solid.

### Step 2: Preparation of 4-methoxycyclohex-1-en-1-yl trifluoromethanesulfonate

A solution of 4-methoxycyclohexan-1-one (500 mg, 3.90 mmol) in anhydrous THF (5 mL) was added dropwise to a solution of LDA (2.34 mL, 4.68 mmol, 2M in THF) in anhydrous THF (10 mL) at −78° C., the mixture was stirred at −78° C. for 0.25 hour. PhNTf₂ (1.53 g, 4.29 mmol) in anhydrous THF (5 mL) was added dropwise to the mixture at −78° C., After stirring for 0.25 hour, the mixture was warmed to 20° C. and stirred at 20° C. for 1.5 hours. The reaction mixture was quenched with saturated aqueous NH₄Cl (25 mL) and extracted EtOAc (30 mL×2). The combined organic layer was dried over MgSO₄, filtered and concentrated. The residue was purified by flash silica gel chromatography (ISCO®; 4 g SepaFlash® Silica Flash Column, Eluent of 0˜10% Ethyl acetate/Petroleum ether gradient @20 mL/min) to give 4-methoxycyclohex-1-en-1-yl trifluoromethanesulfonate (1.05 g, quantitative yield) as yellow oil.

¹H NMR (400 MHz, CDCl₃) δ 1.83-2.03 (2H, m), 2.18-2.29 (1H, m), 2.30-2.41 (1H, m), 2.42-2.54 (2H, m), 3.38 (3H, s), 3.48-3.57 (1H, m), 5.60-5.71 (1H, m).

### Step 3: Preparation of 2-(4-methoxycyclohex-1-en-1-yl)-4,4,5,5-tetramethyl-1, 3,2-dioxaborolane

A mixture of 4-methoxycyclohex-1-en-1-yl trifluoromethanesulfonate (550 mg, 2.11 mmol), Bis-pin (644 mg, 2.54 mmol), KOAc (622 mg, 6.34 mmol) and Pd(dppf)Cl₂·DCM (86 mg, 0.11 mmol) in anhydrous dioxane (5 mL) was degassed and purged with N₂ for 3 times. Then the reaction mixture was stirred at 80° C. for 16 hours under N₂ atmosphere. The reaction mixture was poured into water (25 mL) and extracted with EtOAc (25 mL×3), the combined organic layer was washed with brine (30 mL), dried over anhydrous Na₂SO₄, filtered and concentrated. The residue was purified by flash silica gel chromatography (ISCO®; 12 g SepaFlash® Silica Flash Column, Eluent of 0˜6% Ethyl acetate/Petroleum ether gradient @25 mL/min) to give impure 2-(4-methoxycyclohex-1-en-1-yl)-4,4,5,5-tetramethyl-1,3,2-dioxaborolane (390 mg, yield: 77%) as colorless oil.

¹H NMR (400 MHz, CDCl₃) δ 1.26 (12H, s), 1.49-1.60 (1H, m), 1.87-1.97 (1H, m), 2.06-2.20 (2H, m), 2.26-2.40 (1H, m), 2.41-2.53 (1H, m), 3.38 (3H, s), 3.42-3.51 (1H, m), 6.42-6.51 (1H, m).

### Step 4: Preparation of 1-(4-methoxycyclohex-1-en-1-yl)-8-(1-methyl-1H-pyrazol-4-yl)-3H-pyrazolo[3,4-c]isoquinoline (Compound 8)

A mixture of 1-iodo-8-(1-methyl-1H-pyrazol-4-yl)-3H-pyrazolo[3,4-c]isoquinoline (35 mg, 0.093 mmol), 2-(4-methoxycyclohex-1-en-1-yl)-4,4,5,5-tetramethyl-1,3,2-dioxaborolane (44 mg, 0.19 mmol), Pd(dppf)Cl₂ (6.8 mg, 0.0093 mmol) and Na₂CO₃ (20 mg, 0.19 mmol) in 1, 4-dioxane (2 mL) and H₂O (0.5 mL) was degassed and purged with N₂ for 3 times. The resulting reaction mixture was stirred at 90° C. for 12 hours under N₂ atmosphere. The reaction mixture was poured into water (15 mL) and extracted with EtOAc (15 mL×3). The combined organic layer was washed with brine (20 mL), dried over anhydrous Na₂SO₄, filtered and concentrated. The residue was purified by prep-TLC (DCM/MeOH=10/1), then further purified by prep-HPLC (0.225% FA as an additive) and lyophilized to give Compound 8 (1.22 mg, yield: 3.6%) as a white solid.

¹H NMR (400 MHz, DMSO-d₆) δ 1.89-2.11 (2H, m), 2.34-2.40 (1H, m), 2.59-2.63 (1H, m), 3.26-3.29 (2H, m), 3.41 (3H, s), 3.71-3.80 (1H, m), 3.93 (3H, s), 6.00-6.10 (1H, m), 7.80 (1H, d, J=8.0 Hz), 7.97 (1H, s), 8.18 (1H, d, J=7.6 Hz), 8.27 (1H, s), 8.49 (1H, s), 9.03 (1H, s), 13.71 (1H, brs).

### Example 6. Preparation of Compound 9

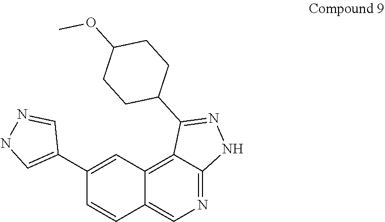 [SMILES: COC1CCC(c2n[nH]c3ncc4ccc(-c5cn[nH]c5)cc4c23)CC1]

A mixture of Compound 8 (36 mg, 0.076 mmol) and PtO₂ (36 mg, 0.16 mmol) in MeOH (40 mL) was degased and purged with H₂ for 3 times and then hydrogenated (50 psi) at 70° C. for 20 hours. The reaction mixture was filtered through a pad of celite and the filtrate was concentrated. The residue was purified by prep-HPLC (0.225% FA as an additive) and lyophilized to give Compound 9 (8.30 mg, yield: 30%) as a light yellow solid.

¹H NMR (400 MHz, DMSO-d₆) δ 1.73-2.25 (8H, m), 3.28 (3H, s), 3.48-3.61 (2H, m), 3.94 (3H, s), 7.83 (1H, d, J=8.4 Hz), 8.05-8.10 (1H, m), 8.20-8.30 (2H, m), 8.39 (1H, s), 9.03 (1H, s), 13.48 (1H, brs).

### Example 7. Preparation of Compound 10

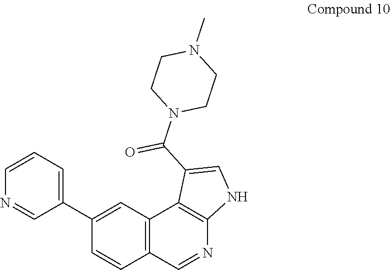 [SMILES: CN1CCN(C(=O)c2c[nH]c3ncc4ccc(-c5cccnc5)cc4c23)CC1]

### Step 1: Preparation of (8-(pyridin-3-yl)-2-(trimethylsilyl)-3H-pyrrolo[2,3-c]isoquinolin-1-yl)methanol

A mixture of Int-4 (260 mg, 0.749 mmol), 3-(trimethylsilyl)prop-2-yn-1-ol (192 mg, 1.50 mmol), Pd(dppf)Cl₂ (55 mg, 0.075 mmol), LiCl (32 mg, 0.075 mmol) and KOAc (147 mg, 1.50 mmol) in anhydrous DMF (6 mL) was degassed and purged with N₂ for 3 times, and then the mixture was stirred at 110° C. for 17 hours under N₂ atmosphere. The reaction mixture was filtered through a pad of celite and the filtrate was concentrated. The residue was purified by Combi Flash (0% to 100% EtOAc in PE) to give (8-(pyridin-3-yl)-2-(trimethylsilyl)-3H-pyrrolo[2,3-c]isoquinolin-1-yl)methanol (290 mg, yield: 60%) as a light yellow solid.

¹H NMR (400 MHz, CD₃OD) δ 0.49 (9H, s), 5.16 (2H, s), 7.55-7.60 (1H, m), 7.80 (1H, d, J=8.4 Hz), 8.21 (1H, d, J=8.4 Hz), 8.29 (1H, d, J=8.0 Hz), 8.59 (1H, d, J=4.8 Hz), 8.85-8.95 (2H, m), 9.00 (1H, d, J=1.6 Hz).

### Step 2: Preparation of (8-(pyridin-3-yl)-3H-pyrrolo[2, 3-c]isoquinolin-1-yl)methanol

To a solution of (8-(pyridin-3-yl)-2-(trimethylsilyl)-3H-pyrrolo[2,3-c]isoquinolin-1-yl)methanol (290 mg, 0.835 mmol) in anhydrous THF (5 mL) was added TBAF (1.67 mmol, 1.67 mL, 1 M in THF) dropwise at 20° C. The mixture was stirred at 20° C. for 7 hours. The reaction mixture was quenched with saturated aqueous NH₄Cl (10 mL), then extracted with EtOAc (10 mL×3). The combined organic layer was washed with saturated aqueous NH₄Cl (10 mL) and brine (10 mL), dried over anhydrous Na₂SO₄, filtered and concentrated. The residue was purified by Combi Flash (0% to 100% EtOAc in PE) to give (8-(pyridin-3-yl)-3H-pyrrolo[2,3-c]isoquinolin-1-yl)methanol (210 mg, yield: 88%) as a light brown solid.

¹H NMR (400 MHz, CD₃OD) δ 5.09 (2H, s), 7.45 (1H, s), 7.61 (1H, dd, J=7.6, 2.8 Hz), 7.84 (1H, dd, J=8.4, 1.2 Hz), 8.25 (1H, d, J=8.8 Hz), 8.32 (1H, d, J=8.0 Hz), 8.60 (1H, d, J=4.0 Hz), 8.82 (1H, s), 8.89 (1H, s), 9.02 (1H, s).

### Step 3: Preparation of 8-(pyridin-3-yl)-3H-pyrrolo[2,3-c]isoquinoline-1-carbaldehyde

To a solution of give (8-(pyridin-3-yl)-3H-pyrrolo[2,3-c]isoquinolin-1-yl)methanol (210 mg, 0.763 mmol) in anhydrous DCM (5 mL) was added Dess-Martin periodinane (1.29 g, 3.05 mmol), then the mixture was stirred at 20° C. for 2 hours. The reaction mixture was filtered through a pad of celite and the filtrate was concentrated. The residue was purified by Combi Flash (0% to 100% EtOAc in PE) to give 8-(pyridin-3-yl)-3H-pyrrolo[2,3-c]isoquinoline-1-carbaldehyde (150 mg, yield: 66%) as a light yellow solid.

¹H NMR (400 MHz, CD₃OD) δ 7.90-8.00 (3H, m), 8.14 (1H, d, J=7.6 Hz), 8.26 (1H, d, J=8.4 Hz), 8.32-8.36 (1H, m), 8.60-8.65 (1H, m), 9.03 (1H, s), 9.07 (1H, d, J=2.0 Hz), 9.91 (1H, s), 9.98 (1H, s).

### Step 4: Preparation of 8-(pyridin-3-yl)-3H-pyrrolo[2,3-c]isoquinoline-1-carboxylic acid

To a solution of NaClO₂ (299 mg, 3.29 mmol) and NaH₂PO₄ (659 mg, 5.49 mmol) in H₂O (4 mL) was added dropwise a solution of 8-(pyridin-3-yl)-3H-pyrrolo[2,3-c]isoquinoline-1-carbaldehyde (150 mg, 0.549 mmol) and 2-methylbut-2-ene (1.92 g, 27.4 mmol) in t-BuOH (1 mL) and THF (4 mL) at 0° C. The mixture was stirred at 0° C. for 6 hours, then at 20° C. for 12 hours. The reaction mixture was diluted with H₂O (10 mL) and extracted with EtOAc (5 mL×4). The combined organic layer was washed with H₂O (5 mL) and brine (8 mL), dried over anhydrous Na₂SO₄, filtered and concentrated to give 8-(pyridin-3-yl)-3H-pyrrolo[2,3-c]isoquinoline-1-carboxylic acid (150 mg, yield: 73%) as a light gray solid.

¹H NMR (400 MHz, CD₃OD) δ 6.80 (1H, s), 7.05-7.10 (1H, m), 7.12 (1H, s), 7.15-7.20 (2H, m), 7.30-7.35 (1H, m), 7.35-7.40 (2H, m), 7.45 (1H, s), 7.47 (1H, s), 7.50-7.55 (1H, m).

### Step 5: Preparation of (4-methylpiperazin-1-yl)(8-(pyridin-3-yl)-3H-pyrrolo[2,3-c]isoquinolin-1-yl)methanone (Compound 10)

To a solution of 8-(pyridin-3-yl)-3H-pyrrolo[2,3-c]isoquinoline-1-carboxylic acid (130 mg, 0.449 mmol) and 1-methylpiperazine (90 mg, 0.899 mmol) in anhydrous DMF (3 mL) was added DIPEA (174 mg, 1.35 mmol), HOBt (182 mg, 1.35 mmol) and EDCI (215 mg, 1.12 mmol) at 20° C. The mixture was stirred at 20° C. for 24 hours, then at 50° C. for 5 hours. The reaction mixture was diluted with H₂O (10 mL) and extracted with EtOAc (20 mL×4). The combined organic layer was washed with H₂O (10 mL) and brine (10 mL), dried over anhydrous Na₂SO₄, filtered and concentrated. The residue was purified by prep-HPLC (0.225% FA as an additive) to give Compound 10 (2.11 mg, yield: 1%) as a light gray solid.

¹H NMR (400 MHz, CDCl₃) δ 1.25-1.40 (4H, m), 2.35-2.50 (4H, m), 3.70-3.80 (3H, m), 7.45-7.50 (1H, m), 7.56 (1H, s), 7.79 (1H, dd, J=8.4, 1.6 Hz), 8.05 (1H, d, J=8.0 Hz), 8.19 (1H, d, J=8.4 Hz), 8.58 (1H, s), 8.69 (1H, d, J=4.8 Hz), 9.01 (1H, s), 9.03 (1H, d, J=2.0 Hz), 9.75 (1H, brs).

### Example 8. Preparation of Compound 11

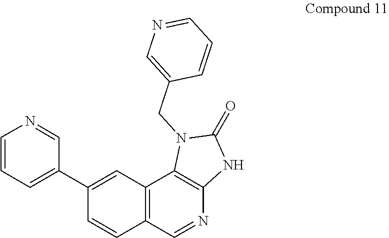 [SMILES: O=c1[nH]c2ncc3ccc(-c4cccnc4)cc3c2n1Cc1cccnc1]

### Step 1: Preparation of 2,2,2-trichloroethyl (pyridin-3-ylmethyl)carbamate

To a solution of pyridin-3-ylmethanamine (1.00 g, 9.25 mmol) and Et₃N (1.87 g, 18.5 mmol) in anhydrous DCM (10 mL) was added Troc-C1 (2.55 g, 12.0 mmol) at 0° C. The mixture was stirred at 0° C. for 1 hour, then stirred at 25° C. and for another 1 hour. The reaction mixture was quenched with water (50 mL) and extracted with DCM (100 mL×2). The combined organic layer was dried over anhydrous Na₂SO₄, filtered and concentrated. The residue was purified by Combi Flash (0% to 100% EtOAc in PE) to give 2,2,2-trichloroethyl (pyridin-3-ylmethyl)carbamate (747 mg, yield: 29%) as yellow oil.

¹H NMR (400 MHz, CDCl₃) δ 4.48 (2H, d, J=6.4 Hz), 4.79 (2H, s), 5.40 (1H, brs), 7.29-7.34 (1H, m), 7.69 (1H, d, J=8.0 Hz), 8.56-8.63 (2H, m).

### Step 2: Preparation of 1-(4-iodo-6-(pyridin-3-yl)isoquinolin-3-yl)-3-(pyridin-3-ylmethyl)urea

A mixture of Int-4 (200 mg, 0.576 mmol) and 2,2,2-trichloroethyl (pyridin-3-ylmethyl)carbamate (196 mg, 0.691 mmol) in DMF (4 mL) was added NaH (69 mg, 1.73 mmol, 60% dispersion in mineral oil) at 0° C., then the reaction mixture was stirred at 0° C. for 2 hours. The reaction mixture was quenched with saturated aqueous NH₄Cl (5 mL), then diluted with water (10 mL) and extracted with EtOAc (25 mL×2). The combined organic layer was washed with brine (50 mL), dried over anhydrous Na₂SO₄, filtered and concentrated. The residue was purified by Combi Flash (0% to 10% MeOH in DCM) to yield 1-(4-iodo-6-(pyridin-3-yl)isoquinolin-3-yl)-3-(pyridin-3-ylmethyl)urea (60 mg, yield: 22%) as a yellow solid.

### Step 3: Preparation of 8-(pyridin-3-yl)-1-(pyridin-3-ylmethyl)-1,3-dihydro-2H-imidazo[4,5-c]isoquinolin-2-one (Compound 11)

A mixture of 1-(4-iodo-6-(pyridin-3-yl)isoquinolin-3-yl)-3-(pyridin-3-ylmethyl)urea (50 mg, 0.10 mmol), Pd₂(dba)₃ (10 mg, 0.010 mmol), Xantphos (12 mg, 0.021 mmol) and Cs₂CO₃ (68 mg, 0.21 mmol) in anhydrous dioxane (2 mL) was degassed and purged with N₂ for 3 times. Then the mixture was stirred at 100° C. for 12 hours under N₂ atmosphere. The reaction mixture was diluted with water (10 mL) and extracted with EtOAc (25 mL×2). The combined organic layer was washed with brine (25 mL), dried over anhydrous Na₂SO₄, filtered and concentrated. The residue was purified by prep-HPLC (0.05% HCOONH₄ as an additive) and lyophilized to give Compound 11 (11.25 mg, yield: 25%) as an off-white solid.

¹H NMR (400 MHz, CDCl₃) δ 5.66 (2H, s), 7.30-7.35 (1H, m), 7.40-7.45 (1H, m), 7.60-7.70 (3H, m), 7.94 (1H, s), 8.12 (1H, d, J=8.8 Hz), 8.62 (1H, d, J=4.4 Hz), 8.70-8.75 (3H, m), 8.90 (1H, s), 9.25 (1H, brs).

### Example 9. Preparation of Compound 12

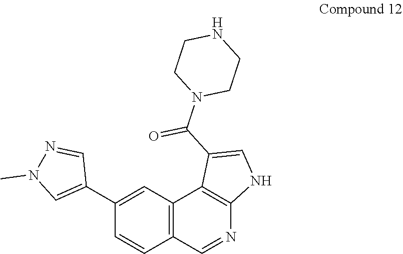 [SMILES: Cn1cc(-c2ccc3cnc4[nH]cc(C(=O)N5CCNCC5)c4c3c2)cn1]

### Step 1: Preparation of (8-(1-methyl-1H-pyrazol-4-yl)-2-(trimethylsilyl)-3H-pyrrolo[2,3-c]isoquinolin-1-yl)methanol

A mixture of Int-5 (540 mg, 1.54 mmol), 3-(trimethylsilyl)prop-2-yn-1-ol (396 mg, 3.08 mmol), Pd(dppf)Cl₂ (113 mg, 0.154 mmol), KOAc (303 mg, 3.08 mmol) and LiCl (65 mg, 1.5 mmol) in anhydrous DMF (20 mL) was degassed and purged with N₂ for 3 times, and then the mixture was stirred at 110° C. for 12 hours under N₂ atmosphere. The reaction mixture was filtered through a pad of celite and the solid was washed with EtOAc (3 mL×2). The filtrate was concentrated and the residue was purified by Combi Flash (0% to 100% EtOAc in PE) to give compound 6 (290 mg, yield: 45%) as a light brown solid.

¹H NMR (400 MHz, CD₃OD) δ 0.49 (9H, s), 3.97 (3H, s), 5.15 (2H, s), 7.60 (1H, s), 7.65-7.75 (2H, m), 7.97 (1H, s), 8.71 (1H, s), 8.75 (1H, s).

### Step 2: Preparation of (8-(1-methyl-1H-pyrazol-4-yl)-3H-pyrrolo[2, 3-c]isoquinolin-1-yl)methanol

To a solution of (8-(1-methyl-1H-pyrazol-4-yl)-2-(trimethylsilyl)-3H-pyrrolo[2,3-c]isoquinolin-1-yl)methanol (290 mg, 0.827 mmol) in anhydrous THF (5 mL) was added TBAF (1.65 mL, 1.65 mmoL, 1 M in THF) dropwise at 20° C. The mixture was stirred at 20° C. for 28 hours. The reaction mixture was filtered and the filtrate was concentrated. The residue was purified by Combi Flash (0% to 100% EtOAc in PE) to give (8-(1-methyl-1H-pyrazol-4-yl)-3H-pyrrolo[2,3-c]isoquinolin-1-yl)methanol (170 mg, yield: 68%) as a light gray solid.

¹H NMR (400 MHz, CD₃OD) δ 3.98 (3H, s), 5.08 (2H, s), 7.39 (1H, s), 7.73 (1H, dd, J=8.8, 1.6 Hz), 8.05 (1H, s), 8.08 (1H, d, J=8.4 Hz), 8.20 (1H, s), 8.64 (1H, s), 8.77 (1H, s).

### Step 3: Preparation of 8-(1-methyl-1H-pyrazol-4-yl)-3H-pyrrolo[2,3-c]isoquinoline-1-carbaldehyde

To a solution of (8-(1-methyl-1H-pyrazol-4-yl)-3H-pyrrolo[2,3-c]isoquinolin-1-yl)methanol (70 mg, 0.25 mmol) in anhydrous DCM (3 mL) was added Dess-Martin periodinane (213 mg, 0.503 mmol) at 20° C. The mixture was stirred at 20° C. for 3 hours. The reaction mixture was filtered through a pad of celite and the filtrate was concentrated. The residue was purified by Combi Flash (0% to 100% EtOAc in PE) to give 8-(1-methyl-1H-pyrazol-4-yl)-3H-pyrrolo[2,3-c]isoquinoline-1-carbaldehyde (110 mg, yield: 64%) as a light gray solid.

### Step 4: Preparation of 8-(1-methyl-1H-pyrazol-4-yl)-3H-pyrrolo[2,3-c]isoquinoline-1-carboxylic acid

To a solution of NaClO₂ (216 mg, 2.39 mmol) and NaH₂PO₄ (478 mg, 3.98 mmol) in water (2 mL) was added dropwise a solution of 8-(1-methyl-1H-pyrazol-4-yl)-3H-pyrrolo[2,3-c]isoquinoline-1-carbaldehyde (110 mg, 0.398 mmol) and 2-methylbut-2-ene (1.40 g, 19.9 mmol) in t-BuOH (0.5 mL) and THF (2 mL) at 0° C. The mixture was stirred at 0° C. for 4 hours, then at 20° C. for 70 hours. The reaction mixture was diluted with water (5 mL) and basified with saturated aqueous NaHCO₃ to pH=8 then washed with EtOAc (5 mL). The aqueous layer was acidified with 1M aqueous HCl to pH=3, then extracted with EtOAc (5 mL×3). The combined organic layer was washed with brine (6 mL), dried over anhydrous Na₂SO₄, filtered and concentrated to give 8-(1-methyl-1H-pyrazol-4-yl)-3H-pyrrolo[2,3-c]isoquinoline-1-carboxylic acid (80 mg, yield: 59%) as a light brown solid.

### Step 5: Preparation of tert-butyl 4-(8-(1-methyl-1H-pyrazol-4-yl)-3H-pyrrolo[2,3-c]isoquinoline-1-carbonyl)piperazine-1-carboxylate

To a solution of 8-(1-methyl-1H-pyrazol-4-yl)-3H-pyrrolo[2,3-c]isoquinoline-1-carboxylic acid (70 mg, 0.24 mmol) and 1-Boc-piperazine (107 mg, 0.479 mmol) in anhydrous DMF (3 mL) was added DIPEA (124 mg, 0.958 mmol), HOBt (97 mg, 0.72 mmol) and EDCI (115 mg, 0.599 mmol). The mixture was stirred at 20° C. for 16 hours. The reaction mixture was concentrated and the residue was diluted with water (8 mL) and extracted with EtOAc (8 mL×3). The combined organic layer was washed with brine (10 mL), dried over anhydrous Na₂SO₄, filtered and concentrated. The residue was purified by Combi Flash (0% to 6% MeOH in DCM) to give tert-butyl 4-(8-(1-methyl-1H-pyrazol-4-yl)-3H-pyrrolo[2,3-c]isoquinoline-1-carbonyl)piperazine-1-carboxylate (30 mg, yield: 25%) as a light brown solid.

### Step 6: Preparation of (8-(1-methyl-1H-pyrazol-4-yl)-3H-pyrrolo[2, 3-c]isoquinolin-1-yl)(piperazin-1-yl)methanone (Compound 12)

A mixture of tert-butyl 4-(8-(1-methyl-1H-pyrazol-4-yl)-3H-pyrrolo[2,3-c]isoquinoline-1-carbonyl)piperazine-1-carboxylate (30 mg, 0.065 mmol) in 4M HCl/dioxane (2 mL) was stirred at 25° C. for 1 hour. The reaction mixture was concentrated and the residue was purified by prep-HPLC (0.225% FA as an additive), then lyophilized to give Compound 12 (3.37 mg, yield: 12%, HCl salt) as a yellow solid.

¹H NMR (400 MHz, D₂O) δ 3.25-3.35 (4H, m), 3.86 (3H, s), 3.90-4.00 (4H, m), 7.40 (1H, d, J=8.4 Hz), 7.64 (1H, s), 7.67 (1H, s), 7.72 (1H, s), 7.77 (1H, s), 7.85 (1H, d, J=8.8 Hz), 8.63 (1H, s).

### Example 10. Preparation of Compound 13

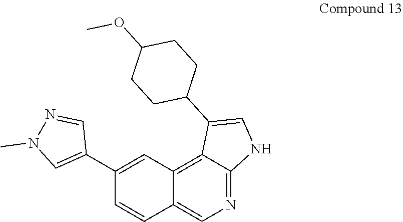 [SMILES: COC1CCC(c2c[nH]c3ncc4ccc(-c5cnn(C)c5)cc4c23)CC1]

### Step 1: Preparation of 1-(4-(benzyloxy)cyclohexyl)-8-(1-methyl-1H-pyrazol-4-yl)-2-(trimethylsilyl)-3H-pyrrolo[2,3-c]isoquinoline

A mixture of Int-5 (470 mg, 1.34 mmol), Int-6 (769 mg, 2.68 mmol), Pd(dppf)Cl₂ (98 mg, 0.13 mmol), KOAc (263 mg, 2.68 mmol) and LiCl (57 mg, 1.34 mmol) in DMF (10 mL) was degassed and purged with N₂ for 3 times, and then the mixture was stirred at 110° C. for 16 hours under N₂ atmosphere. The reaction mixture was concentrated under reduced pressure and the residue was purified by Combi Flash (0% to 10% EtOAc in PE) to give 1-(4-(benzyloxy)cyclohexyl)-8-(1-methyl-1H-pyrazol-4-yl)-2-(trimethylsilyl)-3H-pyrrolo[2,3-c]isoquinoline (700 mg, yield: 83%) as a light brown solid.

¹H NMR (400 MHz, DMSO-d₆) δ 0.40-0.42 (9H, m), 1.40-1.60 (2H, m), 1.70-1.90 (2H, m), 2.00-2.10 (1H, m), 2.20-2.40 (3H, m), 3.60-3.70 (1H, m), 3.91 (3H, s), 4.00-4.10 (1H, m), 4.65 (2H, s), 7.30-7.50 (4H, m), 7.69 (1H, d, J=8.4 Hz), 7.90-7.95 (2H, m), 8.12 (1H, d, J=8.4 Hz), 8.25 (1H, s), 8.45 (1H, s), 8.83 (1H, s), 11.36 (1H, brs).

### Step 2: Preparation of 1-(4-(benzyloxy)cyclohexyl)-8-(1-methyl-1H-pyrazol-4-yl)-3H-pyrrolo[2,3-c]isoquinoline

To a solution of 1-(4-(benzyloxy)cyclohexyl)-8-(1-methyl-1H-pyrazol-4-yl)-2-(trimethylsilyl)-3H-pyrrolo[2,3-c]isoquinoline (650 mg, 1.28 mmol) in THF (10 mL) was added TBAF (2.56 mL, 2.56 mmol, 1M in THF) at 20° C. The mixture was stirred at 20° C. for 40 hours. The reaction mixture was diluted with water (30 mL) and extracted with EtOAc (20 mL×3). The combined organic layer was washed with water (20 mL) and brine (20 mL), dried over anhydrous Na₂SO₄, filtered and concentrated to give 1-(4-(benzyloxy)cyclohexyl)-8-(1-methyl-1H-pyrazol-4-yl)-3H-pyrrolo[2,3-c]isoquinoline (550 mg, yield: 98%) as alight yellow solid.

¹H NMR (400 MHz, DMSO-d₆) δ 1.10-1.40 (3H, m), 1.50-1.70 (2H, m), 1.80-1.90 (1H, m), 1.93-2.10 (1H, m), 2.20-2.30 (1H, m), 3.10-3.15 (1H, m), 3.40-3.50 (1H, m), 3.93 (3H, s), 4.60 (2H, s), 7.15-7.25 (1H, m) 7.30-7.40 (4H, m), 7.71 (1H, d, J=8.4 Hz), 8.02 (1H, s), 8.11 (1H, d, J=8.4 Hz), 8.29 (1H, d, J=5.2 Hz), 8.31 (1H, s), 8.79 (1H, s), 11.71 (1H, brs).

### Step 3: Preparation of 1-(4-(benzyloxy)cyclohexyl)-8-(1-methyl-1H-pyrazol-4-yl)-3-tosyl-3H-pyrrolo[2,3-c]isoquinoline

To a solution of 1-(4-(benzyloxy)cyclohexyl)-8-(1-methyl-1H-pyrazol-4-yl)-3H-pyrrolo[2,3-c]isoquinoline (180 mg, 0.412 mmol) in anhydrous THF (5 mL) was added NaH (33 mg, 0.82 mmol, 60% dispersion in mineral oil) at 0° C. After stirring at 0° C. for 0.2 hour, TosCl (173 mg, 0.907 mmol) was added to the above solution at 0° C. The mixture was stirred at 0° C. for 1 hour. The reaction mixture was quenched with water (5 mL) at 0° C. and then extracted with EtOAc (10 mL×2). The combined organic layer was washed with brine (10 mL), dried over anhydrous Na₂SO₄, filtered and concentrated to give 1-(4-(benzyloxy)cyclohexyl)-8-(1-methyl-1H-pyrazol-4-yl)-3-tosyl-3H-pyrrolo[2,3-c]isoquinoline (200 mg, yield: 77%) as a yellow solid.

¹H NMR (400 MHz, DMSO-d₆) δ 1.45-1.94 (5H, m), 2.05-2.15 (1H, m), 2.15-2.28 (3H, m), 2.31 (3H, s), 3.45-3.50 (1H, m), 3.91-3.96 (3H, m), 4.52-4.62 (2H, m), 7.33-7.42 (6H, m), 7.56-7.59 (1H, m), 7.87 (1H, d, J=8.4 Hz), 8.00-8.05 (3H, m), 8.06 (1H, s), 8.19 (1H, d, J=8.4 Hz), 8.24-8.29 (1H, m), 8.32-8.38 (1H, m), 8.97 (1H, s).

### Step 4: Preparation of 4-(8-(1-methyl-1H-pyrazol-4-yl)-3-tosyl-3H-pyrrolo[2,3-c]isoquinolin-1-yl)cyclohexan-1-ol

A solution of 1-(4-(benzyloxy)cyclohexyl)-8-(1-methyl-1H-pyrazol-4-yl)-3-tosyl-3H-pyrrolo[2,3-c]isoquinoline (200 mg, 0.339 mmol) in TFA (3 mL) was stirred at 90° C. for 1 hour. The reaction mixture was concentrated then the residue was dissolved into MeOH and treated with K₂CO₃ to give 4-(8-(1-methyl-1H-pyrazol-4-yl)-3-tosyl-3H-pyrrolo[2,3-c]isoquinolin-1-yl)cyclohexan-1-ol (170 mg, yield: 77%).

¹H NMR (400 MHz, DMSO-d₆) δ 1.45-1.65 (3H, m), 1.78-1.94 (2H, m), 1.90-2.05 (2H, m), 2.10-2.20 (2H, m), 2.31 (3H, s), 3.93 (3H, s), 4.38-4.73 (1H, m), 7.12-7.28 (1H, m), 7.28-7.34 (1H, m), 7.39 (1H, d, J=8.4 Hz), 7.57 (1H, s), 7.67-7.91 (1H, m), 7.96-8.07 (2H, m), 8.19 (1H, d, J=8.4 Hz), 8.23-8.32 (1H, m), 8.34 (1H, s), 8.72-9.02 (1H, m).

### Step 5: Preparation of 1-(4-methoxycyclohexyl)-8-(1-methyl-1H-pyrazol-4-yl)-3-tosyl-3H-pyrrolo[2,3-c]isoquinoline

To a solution of 4-(8-(1-methyl-1H-pyrazol-4-yl)-3-tosyl-3H-pyrrolo[2,3-c]isoquinolin-1-yl)cyclohexan-1-ol (170 mg, 0.340 mmol) in anhydrous DMF (5 mL) was added NaH (54 mg, 1.36 mmol, 60% dispersion in mineral oil) at 0° C. After stirring at 0° C. for 0.5 hour, MeI (193 mg, 1.36 mmol) was added to the above solution. The mixture was stirred at 0° C. for another 16.5 hours. The reaction mixture was quenched with MeOH (5 mL) at 20° C. and concentrated. The residue was purified by Combi Flash (0% to 100% EtOAc in PE) to give 1-(4-methoxycyclohexyl)-8-(1-methyl-1H-pyrazol-4-yl)-3-tosyl-3H-pyrrolo[2,3-c]isoquinoline (50 mg, yield: 28%) as a light yellow solid.

### Step 6: Preparation of 1-(4-methoxycyclohexyl)-8-(1-methyl-1H-pyrazol-4-yl)-3H-pyrrolo[2, 3-c]isoquinoline (Compound 13)

To a solution of 1-(4-methoxycyclohexyl)-8-(1-methyl-1H-pyrazol-4-yl)-3-tosyl-3H-pyrrolo[2,3-c]isoquinoline (50 mg, 0.097 mmol) in THF (2 mL), MeOH (1 mL) and H₂O (0.5 mL) was added KOH (16 mg, 0.029 mmol) at 20° C. The mixture was stirred at 60° C. for 2 hours. The reaction mixture was diluted with water (5 mL) and extracted with EtOAc (10 mL×3). The combined organic layer was washed with brine (5 mL), dried over anhydrous Na₂SO₄, filtered and concentrated. The residue was purified by pre-HPLC (0.225% FA as an additive), then lyophilized to give Compound 13 (3.74 mg, yield: 10%) as a white solid.

¹H NMR (400 MHz, DMSO-d₆) δ 1.40-1.60 (4H, m), 2.10-2.20 (4H, m), 3.20-3.30 (5H, m), 3.94 (3H, s), 7.19 (1H, d, J=2.4 Hz), 7.72 (1H, dd, J=8.4, 1.6 Hz), 8.00 (1H, s), 8.11 (1H, d, J=8.4 Hz), 8.25-8.40 (2H, m), 8.79 (1H, s), 11.72 (1H, brs).

### Example 11. Preparation of Compound 14

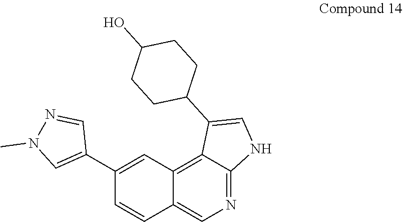 [SMILES: Cn1cc(-c2ccc3cnc4[nH]cc(C5CCC(O)CC5)c4c3c2)cn1]

A mixture of 1-(4-(benzyloxy)cyclohexyl)-8-(1-methyl-1H-pyrazol-4-yl)-3H-pyrrolo[2,3-c]isoquinoline (100 mg, 0.229 mmol) in TFA (3 mL) was stirred at 90° C. for 14 hours. The reaction mixture was concentrated and the residue was dissolved in MeOH and treated with K₂CO₃, then filtered and the filtrate was concentrated. The residue was purified by pre-HPLC (0.225% FA as an additive), then lyophilized to give Compound 14 (60 mg, yield: 75%) as a light yellow solid.

¹H NMR (400 MHz, DMSO-d₆) δ 1.40-1.65 (3H, m), 1.75-2.00 (2H, m), 2.00-2.20 (3H, m), 3.50-3.60 (1H, m), 3.93 (3H, s), 4.40-4.70 (1H, m), 7.15-7.20 (1H, m), 7.65-7.75 (1H, m), 7.95-8.05 (1H, m), 8.10-8.15 (1H, m), 8.25-8.40 (2H, m), 8.79 (1H, s), 11.70 (1H, brs).

### Example 12. Preparation of Compound 15, 16 and 17

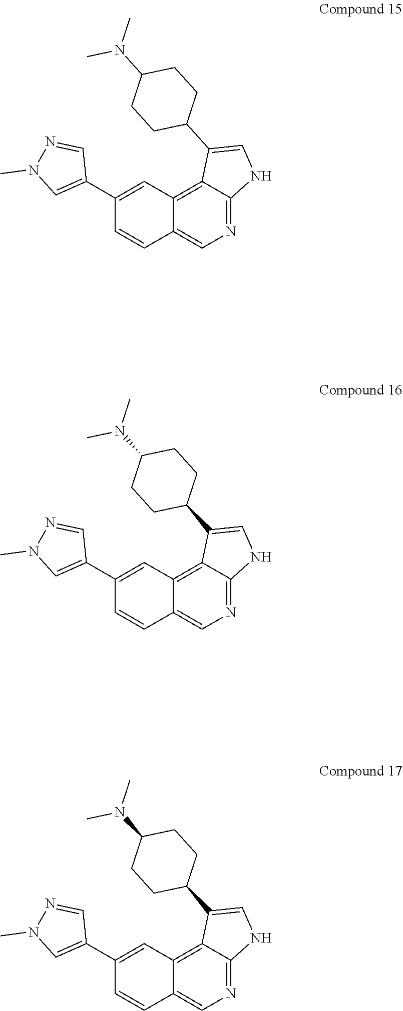 [SMILES: CN(C)C1CCC(c2c[nH]c3ncc4ccc(-c5cnn(C)c5)cc4c23)CC1 ; Cn1cc(-c2ccc3cnc4[nH]cc([C@H]5CC[C@H](N(C)C)CC5)c4c3c2)cn1 ; Cn1cc(-c2ccc3cnc4[nH]cc([C@H]5CC[C@@H](N(C)C)CC5)c4c3c2)cn1]

### Step 1: Preparation of 4-(8-(1-methyl-1H-pyrazol-4-yl)-3H-pyrrolo[2, 3-c]isoquinolin-1-yl)cyclohexan-1-one

To a solution of Compound 14 (200 mg, 0.577 mmol) in DCM (5 mL) was added Dess-Martin periodinane (490 mg, 1.15 mmol) at 20° C. The mixture was stirred at 20° C. for 1 hour. The reaction mixture was quenched with saturated aqueous NaHCO₃ (10 mL) at 20° C. and then extracted with DCM (10 mL×3). The combined organic layer was washed with brine (15 mL), dried over anhydrous Na₂SO₄, filtered and concentrated. The residue was purified by Combi Flash (0% to 100% EtOAc in PE) to give 4-(8-(1-methyl-1H-pyrazol-4-yl)-3H-pyrrolo[2,3-c]isoquinolin-1-yl)cyclohexan-1-one (160 mg, yield: 72%) as a yellow solid.

¹H NMR (400 MHz, DMSO-d₆) δ 1.80-1.95 (2H, m), 2.15-2.40 (4H, m), 2.80-2.90 (2H, m), 3.80-3.90 (1H, m), 3.93 (3H, s), 7.26 (1H, d, J=2.4 Hz), 7.72-7.78 (1H, m), 8.00-8.06 (1H, m), 8.12 (1H, s), 8.30-8.40 (2H, m), 8.81 (1H, s), 11.79 (1H, brs).

### Step 2: Preparation of N,N-dimethyl-4-(8-(1-methyl-1H-pyrazol-4-yl)-3H-pyrrolo[2,3-c]isoquinolin-1-yl)cyclohexan-1-amine (Compound 15)

To a solution of N,N-dimethylamine HCl salt (152 mg, 1.86 mmol) in DCE (5 mL) was added TEA (235 mg, 2.32 mmol) at 20° C. and the reaction mixture was stirred at 20° C. for 0.5 hour. To the reaction mixture were added 4-(8-(1-methyl-1H-pyrazol-4-yl)-3H-pyrrolo[2,3-c]isoquinolin-1-yl)cyclohexan-1-one (160 mg, 0.465 mmol) and HOAc (56 mg, 0.93 mmol), followed by NaBH(OAc)₃ (197 mg, 0.929 mmol) added at 20° C. The mixture was stirred at 20° C. for another 16.5 hours. The reaction mixture was quenched with saturated aqueous NaHCO₃ (10 mL) at 20° C. and then extracted with DCM (10 mL×3). The combined organic layer was washed with brine (10 mL), dried over anhydrous Na₂SO₄, filtered and concentrated.

The residue was purified by prep-HPLC (0.225% FA as an additive), then lyophilized to give Compound 15 (30 mg, yield: 17%, FA salt) as a yellow solid.

¹H NMR (400 MHz, DMSO-d₆) δ 1.50-1.60 (1H, m), 1.60-1.85 (2H, m), 1.86-2.10 (5H, m), 2.25-2.40 (6H, m), 2.60-3.40 (1H, m), 3.80-3.90 (1H, m), 3.90-3.95 (3H, m), 7.20-7.25 (1H, m), 7.71 (1H, dd, J=8.8, 1.6 Hz), 8.10-8.15 (1H, m), 8.20-8.40 (3H, m), 8.75-8.80 (1H, m), 11.70-11.75 (1H, m).

### Step 3: SFC Purification

Compound 15 (30 mg, FA salt) was submitted for SFC separation and lyophilized to give Compound 16 (7.31 mg, yield: 24%, trans isomer) as a yellow solid and Compound 17 (14.74 mg, yield: 49%, cis isomer) as a yellow solid.

Compound 16: ¹H NMR (400 MHz, DMSO-d₆) δ 1.41-1.55 (2H, m), 1.56-1.70 (2H, m), 1.95-2.15 (2H, m), 2.20-2.30 (2H, m), 2.33 (6H, s), 2.35-2.45 (1H, m), 3.20-3.30 (1H, m), 3.93 (3H, s), 7.19 (1H, s), 7.70 (1H, d, J=8.4 Hz), 8.00 (1H, s), 8.11 (1H, d, J=8.4 Hz), 8.25-8.35 (2H, m), 8.79 (1H, s), 11.73 (1H, brs).

Compound 17: ¹H NMR (400 MHz, DMSO-d₆) δ 1.60-1.75 (2H, m), 1.80-1.90 (4H, m), 1.95-2.05 (2H, m), 2.10-2.15 (1H, m), 2.20 (6H, s), 3.35-3.45 (1H, m), 3.93 (3H, s), 7.21 (1H, d, J=2.0 Hz), 7.70 (1H, d, J=8.4 Hz), 8.02 (1H, s), 8.10 (1H, d, J=8.4 Hz), 8.25-8.35 (2H, m), 8.78 (1H, s), 11.70 (1H, brs).

The following compounds were synthesized analogously to Compounds 15, 16, and 17

| Comp. | | | |
|---|---|---|---|
| No | Structure | Name | ¹H NMR (400 MHz) |
| 48 | 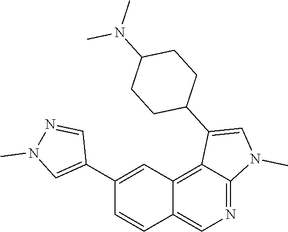 | N,N-dimethyl-4-(3- methyl-8-(1- methyl-1H-pyrazol- 4-yl)-3H- pyrrolo[2,3- c]isoquinolin-1- yl)cyclohexan-1- amine | — |
| 49 | 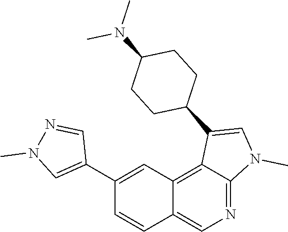 | (1s,4s)-N,N- dimethyl-4-(3- methyl-8-(1- methyl-1H-pyrazol- 4-yl)-3H- pyrrolo[2,3- c]isoquinolin-1- yl)cyclohexan-1- amine | D₂O; δ 1.40-1.50 (8H, m), 2.35- 2.30 (1H, m), 2.55 (6H, s), 2.85- 2.95 (1H, m), 3.61 (3H, s), 3.67 (3H, s), 6.53 (1H, d, J = 8.4 Hz), 6.65 (1H, s), 6.78 (1H, s), 7.00- 7.05 (3H, m), 7.93 (1H, s), 8.42 (1H, s). |
| 50 | 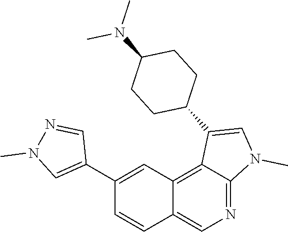 | (1r,4r)-N,N- dimethyl-4-(3- methyl-8-(1- methyl-1H-pyrazol- 4-yl)-3H- pyrrolo[2,3- c]isoquinolin-1- yl)cyclohexan-1- amine | D₂O; δ 0.59-0.87 (4H, m), 1.20- 1.30 (2H, m), 1.39-1.52 (1H, m), 1.55-1.60 (2H, m), 2.53 (6H, s), 2.61-2.77 (1H, m), 3.53 (3H, s), 3.71 (3H, s), 6.43 (1H, s), 6.50 (1H, s), 6.66 (1H, d, J = 8.4 Hz), 6.86 (1H, s), 7.09-7.14 (2H, m), 7.90 (1H, s), 8.41 (1H, s). |

### Example 13. Preparation of Compound 18, 19 and 20

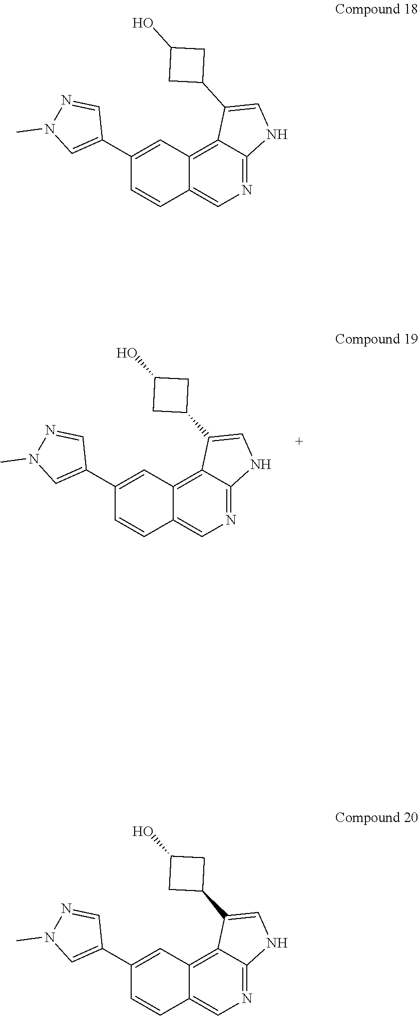 [SMILES: Cn1cc(-c2ccc3cnc4[nH]cc(C5CC(O)C5)c4c3c2)cn1 ; Cn1cc(-c2ccc3cnc4[nH]cc([C@H]5C[C@@H](O)C5)c4c3c2)cn1 ; Cn1cc(-c2ccc3cnc4[nH]cc([C@H]5C[C@H](O)C5)c4c3c2)cn1]

### Step 1: Preparation of 1-(3-(benzyloxy)cyclobutyl)-8-(1-methyl-1H-pyrazol-4-yl)-2-(trimethylsilyl)-3H-pyrrolo[2,3-c]isoquinoline

A mixture of Int-5 (370 mg, 1.06 mmol) and Int-7 (546 mg, 2.11 mmol), LiCl (45 mg, 1.1 mmol), KOAc (207 mg, 2.11 mmol) and Pd(dppf)Cl₂ (77 mg, 0.11 mmol) in anhydrous DMF (15 mL) was degassed and purged with N₂ for 3 times. Then the reaction mixture was stirred at 105° C. for 17 hours under N₂ atmosphere to give a yellow mixture. The reaction mixture was concentrated and the residue was purified by Combi Flash (SiO₂, 0% to 40% EtOAc in PE) to give 1-(3-(benzyloxy)cyclobutyl)-8-(1-methyl-1H-pyrazol-4-yl)-2-(trimethylsilyl)-3H-pyrrolo[2,3-c]isoquinoline (600 mg, yield: 95%) as yellow oil.

¹H NMR (400 MHz, DMSO-d₆) δ 0.45-0.51 (9H, m), 2.70-2.75 (2H, m), 2.85-2.95 (2H, m), 3.60-3.70 (3H, m), 3.95-4.00 (2H, m), 4.25-4.30 (1H, m), 4.40-4.50 (1H, m), 7.35-7.45 (5H, m), 8.00-8.10 (3H, m), 8.20-8.30 (1H, m), 8.75-8.90 (1H, m), 9.15-9.30 (1H, m).

### Step 2: Preparation of 1-(3-(benzyloxy)cyclobutyl)-8-(1-methyl-1H-pyrazol-4-yl)-3H-pyrrolo[2,3-c]isoquinoline

To a mixture of 1-(3-(benzyloxy)cyclobutyl)-8-(1-methyl-1H-pyrazol-4-yl)-2-(trimethylsilyl)-3H-pyrrolo[2,3-c]isoquinoline (600 mg, 1.25 mmol) in THF (15 mL) was added TBAF (2.50 mL, 2.50 mmol, 1M in THF) at 0° C. The reaction mixture was stirred at 25° C. for 4 hours to give a yellow mixture. The reaction mixture was poured into saturated aqueous NH₄Cl (20 mL) and extracted with EtOAc (20 mL×2). The combined organic layer was washed with saturated aqueous NH₄Cl (10 mL×6) and brine (10 mL×2), dried over anhydrous Na₂SO₄, filtered and concentrated to give 1-(3-(benzyloxy)cyclobutyl)-8-(1-methyl-1H-pyrazol-4-yl)-3H-pyrrolo[2,3-c]isoquinoline (500 mg, crude) as a yellow solid.

### Step 3: Preparation of 3-(8-(1-methyl-1H-pyrazol-4-yl)-3H-pyrrolo[2, 3-c]isoquinolin-1-yl)cyclobutan-1-ol (Compound 18)

A solution of 1-(3-(benzyloxy)cyclobutyl)-8-(1-methyl-1H-pyrazol-4-yl)-2-(trimethylsilyl)-3H-pyrrolo[2,3-c]isoquinoline (100 mg, 0.245 mmol) and TFA (140 mg, 1.22 mmol) was stirred at 90° C. for 2 hours to give a black mixture. The mixture was concentrated and the residue was dissolved in MeOH (4 mL), then K₂CO₃ (200 mg, 1.45 mmol) was added and the mixture was stirred at 20° C. for another 0.5 hour to give a yellow mixture. The reaction mixture was filtered and the filtrate was concentrated. The residue was purified by prep-HPLC (0.2% FA as an additive), then lyophilized to give Compound 18 (41 mg, yield: 52% for 2 steps) as a yellow solid.

¹H NMR (400 MHz, DMSO-d₆) δ 1.95-2.05 (1H, m), 2.80-2.90 (1H, m), 3.45-3.55 (1H, m), 3.94 (3H, s), 3.95-4.00 (1H, m), 4.30-4.35 (1H, m), 5.05-5.15 (1H, m), 7.20-7.40 (1H, m), 7.71 (1H, d, J=8.4 Hz), 8.00-8.20 (4H, m), 8.80 (1H, s), 11.72 (1H, brs).

### Step 4: SFC Purification

Compound 18 (30 mg, 0.094 mmol) was submitted for SFC separation and lyophilized to give Compound 19 (14.35 mg, yield: 46%, cis isomer) as a white solid and Compound 20 (10.40 mg, yield: 34%, trans isomer) as a white solid.

Compound 19: ¹H NMR (400 MHz, DMSO-d₆) δ 1.90-2.05 (2H, m), 2.80-2.90 (2H, m), 3.45-3.55 (1H, m), 3.95 (3H, s), 4.20-4.40 (1H, m), 5.11 (1H, d, J=4.8 Hz), 7.24 (1H, s), 7.71 (1H, d, J=8.4 Hz), 8.05-8.15 (2H, m), 8.28 (1H, s), 8.37 (1H, s), 8.79 (1H, s), 11.73 (1H, brs).

Compound 20: ¹H NMR (400 MHz, DMSO-d₆) δ 2.50-2.55 (2H, m), 3.35-3.40 (2H, m), 4.00 (3H, s), 4.02-4.05 (1H, m), 4.30-4.45 (1H, m), 5.20 (1H, d, J=6.0 Hz), 7.42 (1H, s), 7.76 (1H, d, J=8.4 Hz), 8.07 (1H, s), 8.10-8.20 (2H, m), 8.39 (1H, s), 8.85 (1H, s), 11.78 (1H, brs).

### Example 14. Preparation of Compound 21

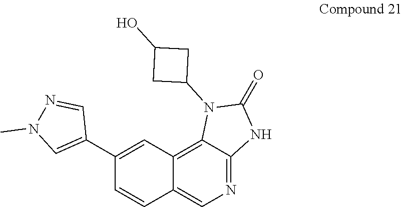 [SMILES: Cn1cc(-c2ccc3cnc4[nH]c(=O)n(C5CC(O)C5)c4c3c2)cn1]

### Step 1: Preparation of 3-(benzyloxy)cyclobutan-1-one oxime

To a solution of 3-(benzyloxy)cyclobutan-1-one (15.0 g, 85.1 mmol) in pyridine (150 mL) was added NH₂OH·HCl (11.8 g, 170 mmol) at 25° C. The mixture was stirred at 25° C. for 12 hours. The reaction mixture was concentrated and the residue was diluted with water (200 mL), then extracted with EtOAc (50 mL×3). The combined organic layer was washed with brine (50 mL), dried over anhydrous Na₂SO₄, filtered and concentrated. The residue was purified by Combi Flash (0% to 20% EtOAc in PE) to give 3-(benzyloxy)cyclobutan-1-one oxime (14.0 g, 86%) as a white solid.

¹H NMR (400 MHz, CDCl₃) δ 2.80-2.93 (2H, m), 3.04-3.13 (1H, m), 3.17-3.26 (1H, m), 4.14-4.21 (1H, m), 4.44 (2H, s), 7.14 (1H, s), 7.23-7.35 (5H, m).

### Step 2: Preparation of 3-(benzyloxy)cyclobutan-1-amine

To a solution of 3-(benzyloxy)cyclobutan-1-one oxime (14.0 g, 73.2 mmol) in anhydrous THF (70 mL) was added LiAlH₄ (6.95 g, 183 mmol) portion-wise at 0° C. After the completion of the addition, the reaction mixture was stirred at 70° C. for 4 hours. The reaction mixture was quenched with water (100 mL) carefully at 0° C. and extracted with EtOAc (50 mL×2). The combined organic layer was washed with brine (50 mL), dried over anhydrous Na₂SO₄, filtered and concentrated to give 3-(benzyloxy)cyclobutan-1-amine (7.00 g, yield: 54%) as red oil.

### Step 3: Preparation of 2,2,2-trichloroethyl (3-(benzyloxy)cyclobutyl)carbamate

To a solution of 3-(benzyloxy)cyclobutan-1-amine (7.00 g, 39.5 mmol) and Et₃N (7.99 g, 79.0 mmol) in anhydrous DCM (30 mL) was added Troc-C1 (12.6 g, 59.2 mmol) dropwise at 0° C., The mixture was stirred at 0° C. for 1 hour. Then the reaction was warmed up to 25° C. and stirred for 11 hours. The reaction mixture was diluted with water (100 mL) and extracted with DCM (50 mL×2). The combined organic layer was washed with brine (50 mL) and dried over anhydrous Na₂SO₄, filtered and concentrated. The residue was purified by Combi Flash (0% to 100% EtOAc in PE) twice to give 2,2,2-trichloroethyl (3-(benzyloxy)cyclobutyl)carbamate (2.10 g, yield: 15%) as colorless oil.

¹H NMR (400 MHz, CDCl₃) δ 1.95-2.30 (2H, m), 2.45-2.60 (1H, m), 2.75-2.80 (1H, m), 3.55-3.70 (1H, m), 3.75-3.90 (1H, m), 4.44 (2H, s), 4.71-4.73 (2H, m), 7.32-7.37 (5H, m).

### Step 4: Preparation of 1-(3-(benzyloxy)cyclobutyl)-3-(4-iodo-6-(1-methyl-1H-pyrazol-4-yl)isoquinolin-3-yl)urea

To a solution of compound 2,2,2-trichloroethyl (3-(benzyloxy)cyclobutyl)carbamate (2.11 g, 6.00 mmol) and Int-5 (1.00 g, 2.86 mmol) in anhydrous DMF (10 mL) was added NaH (571 mg, 14.3 mmol, 60% dispersion in mineral oil) at 0° C., then the mixture was stirred at 25° C. for 12 hours. The reaction mixture was quenched with water (50 mL) and filtered. The crude product was triturated with CH₃CN (10 mL) to give 1-(3-(benzyloxy)cyclobutyl)-3-(4-iodo-6-(1-methyl-1H-pyrazol-4-yl)isoquinolin-3-yl)urea (940 mg, yield: 60%) as a black brown solid.

¹H NMR (400 MHz, DMSO-d₆) δ 1.85-1.96 (1.6H, m), 2.19-2.30 (1.4H, m), 2.61-2.66 (1H, m), 3.78-3.87 (1H, m), 3.90-4.00 (3H, m), 4.16-4.34 (1H, m), 4.40 (2H, s), 7.25-7.45 (5H, m) 7.80-7.90 (2H, m), 7.96 (1H, s), 8.05-8.10 (2H, m), 8.37-8.53 (2H, m), 9.01 (1H, s).

### Step 5: Preparation of 1-(3-(benzyloxy)cyclobutyl)-8-(1-methyl-1H-pyrazol-4-yl)-1,3-dihydro-2H-imidazo[4,5-c]isoquinolin-2-one

A mixture of 1-(3-(benzyloxy)cyclobutyl)-3-(4-iodo-6-(1-methyl-1H-pyrazol-4-yl)isoquinolin-3-yl)urea (900 mg, 1.63 mmol), Pd₂(dba)₃ (149 mg, 0.163 mmol), Xantphos (188 mg, 0.325 mmol) and Cs₂CO₃ (1.06 g, 3.25 mmol) in dioxane (10 mL) was degassed and purged with N₂ for 3 times. Then the mixture was stirred at 100° C. for 12 hours under N₂ atmosphere. The reaction mixture was diluted with water (25 mL) and then extracted with EtOAc (25 mL×2). The combined organic layer was washed with brine (25 mL), dried over anhydrous Na₂SO₄, filtered and concentrated. The residue was purified by Combi Flash (0% to 100% EtOAc in PE) twice to give 1-(3-(benzyloxy)cyclobutyl)-8-(1-methyl-1H-pyrazol-4-yl)-1,3-dihydro-2H-imidazo[4,5-c]isoquinolin-2-one (300 mg, yield: 43%) as a yellow solid.

### Step 6: Preparation of 1-(3-hydroxycyclobutyl)-8-(1-methyl-1H-pyrazol-4-yl)-1,3-dihydro-2H-imidazo[4,5-c]isoquinolin-2-one (Compound 21)

A mixture of compound 1-(3-(benzyloxy)cyclobutyl)-8-(1-methyl-1H-pyrazol-4-yl)-1,3-dihydro-2H-imidazo[4,5-c]isoquinolin-2-one (130 mg, 0.224 mmol) in TFA (10 mL) was stirred at 90° C. for 2 hours. Then the reaction was concentrated and the residue was dissolved in MeOH (10 mL), then K₂CO₃ (47 mg, 0.34 mmol) was added and the mixture was stirred at 25° C. for 4 hours. The reaction mixture was filtered and the filtrate was concentrated.

The residue was purified by Combi Flash (0 to 15% DCM in MeOH), then further purified by prep-HPLC (0.225% FA as an additive) and lyophilized to give Compound 21 (3.0 mg, yield: 12%) as a light yellow solid.

¹H NMR (400 MHz, DMSO-d₆) δ 2.76-2.92 (3H, m), 3.18-3.28 (1H, m), 3.91-3.96 (3H, m), 4.10-4.60 (1H, m), 4.70-4.80 (1H, m), 5.25-5.40 (1H, m), 7.68-7.76 (1H, m), 8.05-8.13 (2H, m), 8.17-8.27 (1H, m), 8.37-8.50 (1H, m), 8.72 (1H, s), 11.68 (1H, brs).

### Example 15. Preparation of Compound 22

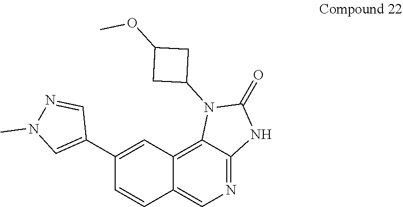 [SMILES: COC1CC(n2c(=O)[nH]c3ncc4ccc(-c5cnn(C)c5)cc4c32)C1]

### Step 1: Preparation of 3-(8-(1-methyl-1H-pyrazol-4-yl)-2-oxo-3-tosyl-2, 3-dihydro-1H-imidazo[4,5-c]isoquinolin-1-yl)cyclobutyl 4-methylbenzenesulfonate

To a solution of Compound 21 (60 mg, 0.18 mmol) and TsCl (102 mg, 0.537 mmol) in anhydrous DMF (2 mL) was added NaH (21 mg, 0.54 mmol, 60% dispersion in mineral oil) at 0° C. Then the reaction mixture was stirred at 25° C. for 2 hours. The reaction mixture was quenched with water (10 mL) and extracted with EtOAc (20 mL×2). The combined organic layer was washed with brine (20 mL), dried over anhydrous Na₂SO₄, filtered and concentrated to give 3-(8-(1-methyl-1H-pyrazol-4-yl)-2-oxo-3-tosyl-2,3-dihydro-1H-imidazo[4,5-c]isoquinolin-1-yl)cyclobutyl 4-methylbenzenesulfonate (150 mg, crude) as yellow oil.

### Step 2: Preparation of 1-(3-methoxycyclobutyl)-8-(1-methyl-1H-pyrazol-4-yl)-1,3-dihydro-2H-imidazo[4,5-c]isoquinolin-2-one (Compound 22)

A mixture of 3-(8-(1-methyl-1H-pyrazol-4-yl)-2-oxo-3-tosyl-2,3-dihydro-1H-imidazo[4,5-c]isoquinolin-1-yl)cyclobutyl 4-methylbenzenesulfonate (150 mg, 0.233 mmol), NaOMe (15 mg, 0.28 mmol) in MeOH (2 mL) was stirred at 25° C. for 6 hours under N₂ atmosphere. The reaction mixture was concentrated and the residue was diluted with water (25 mL) and extracted with EtOAc (25 mL×2). The combined organic layer was dried over anhydrous Na₂SO₄, filtered and concentrated. The residue was purified by prep-HPLC (0.225% FA as an additive) to give Compound 22 (1.50 mg, yield: 1.8%) as an off-white solid.

¹H NMR (400 MHz, CDCl₃) δ 2.58-2.66 (1H, m), 3.11-3.18 (2H, m), 3.40-3.50 (1H, m), 3.65-3.75 (3H, m), 4.04 (3H, s), 4.25-4.40 (0.5H, m), 4.85-4.90 (0.5H, m), 5.03-5.14 (0.5H, m), 5.51-5.67 (0.5H, m), 7.55-7.65 (1H, m), 7.75-8.05 (3H, m), 8.00-8.20 (1H, m), 8.70-8.80 (1H, m).

### Example 16. Preparation of Compound 23

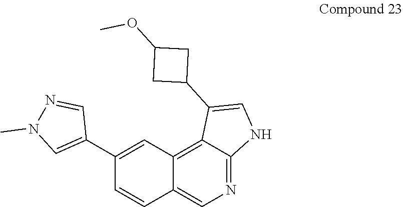 [SMILES: COC1CC(c2c[nH]c3ncc4ccc(-c5cnn(C)c5)cc4c23)C1]

### Step 1: Preparation of 1-(3-(benzyloxy)cyclobutyl)-8-(1-methyl-1H-pyrazol-4-yl)-3-tosyl-3H-pyrrolo[2,3-c]isoquinoline

To a mixture of 1-(3-(benzyloxy)cyclobutyl)-8-(1-methyl-1H-pyrazol-4-yl)-3H-pyrrolo[2,3-c]isoquinoline (250 mg, 0.612 mmol) in anhydrous THF (8 mL) was added NaH (32 mg, 0.080 mmol, 60% dispersion in mineral oil) at 0° C. for and the mixture was stirred at 0° C. for 0.5 hour. Then TsCl (175 mg, 0.918 mmol) was added to the reaction mixture and the reaction was stirred at 20° C. for 3 hours to give a yellow mixture. The reaction mixture was poured into saturated aqueous NH₄Cl (10 mL) and extracted with EtOAc (30 mL×4). The combined organic layer was dried over anhydrous Na₂SO₄, filtered and concentrated. The residue was purified by Combi Flash (SiO₂, 0% to 35% EtOAc in PE) to give 1-(3-(benzyloxy)cyclobutyl)-8-(1-methyl-1H-pyrazol-4-yl)-3-tosyl-3H-pyrrolo[2,3-c]isoquinoline (305 mg, yield: 88%) as yellow gum.

¹H NMR (400 MHz, CDCl₃) δ 1.75-1.85 (1H, m), 2.15-2.20 (1H, m), 2.27 (3H, s), 2.55-2.70 (2H, m), 2.80-2.90 (1H, m), 3.40-3.45 (1H, m), 3.65-3.70 (1H, m), 3.90-4.00 (3H, m), 4.15-4.25 (1H, m), 4.40-4.50 (2H, m), 7.17 (1H, s), 7.20-7.35 (6H, s), 7.50-7.60 (2H, m), 7.67 (1H, s), 7.81 (1H, s), 7.90-8.15 (4H, m), 8.85-8.90 (1H, m).

### Step 2: Preparation of 3-(8-(1-methyl-1H-pyrazol-4-yl)-3-tosyl-3H-pyrrolo[2,3-c]isoquinolin-1-yl)cyclobutan-1-ol

A solution of 1-(3-(benzyloxy)cyclobutyl)-8-(1-methyl-1H-pyrazol-4-yl)-3-tosyl-3H-pyrrolo[2,3-c]isoquinoline (300 mg, 0.533 mmol) in TFA (10 mL) was stirred at 90° C. for 2 hours to give a yellow mixture. The reaction mixture was concentrated and the residue was dissolved in MeOH (10 mL), then K₂CO₃ (368 mg, 2.67 mmol) was added to the mixture at 20° C. The resulting reaction mixture was stirred at 20° C. for another 1 hr to give a yellow mixture. The reaction mixture was poured into saturated aqueous NH₄Cl (40 mL) and extracted with EtOAc (50 mL×3). The combined organic layer was dried over Na₂SO₄, filtered and concentrated to give 3-(8-(1-methyl-1H-pyrazol-4-yl)-3-tosyl-3H-pyrrolo[2,3-c]isoquinolin-1-yl)cyclobutan-1-ol (250 mg, crude) as a yellow solid.

### Step 3: Preparation of 1-(3-methoxycyclobutyl)-8-(1-methyl-1H-pyrazol-4-yl)-3-tosyl-3H-pyrrolo[2,3-c]isoquinoline

To a mixture of 3-(8-(1-methyl-1H-pyrazol-4-yl)-3-tosyl-3H-pyrrolo[2,3-c]isoquinolin-1-yl)cyclobutan-1-ol (100 mg, 0.212 mmol) in anhydrous DMF (2 mL) was added NaH (34 mg, 0.85 mmol, 60% dispersion in mineral oil) at 0° C. The mixture was stirred at 0° C. for 0.5 hour, MeI (120 mg, 0.846 mmol) was added to the mixture and the reaction was stirred at 20° C. for another 2.5 hours to give a yellow mixture. The reaction mixture was poured into saturated aqueous NH₄Cl (10 mL) and extracted with EtOAc (10 mL×4). The combined organic layer was washed with brine (10 mL×2) and water (10 mL×2), dried over anhydrous Na₂SO₄, filtered and concentrated to give 1-(3-methoxycyclobutyl)-8-(1-methyl-1H-pyrazol-4-yl)-3-tosyl-3H-pyrrolo[2,3-c]isoquinoline (100 mg, crude) as yellow gum.

### Step 4: Preparation of 1-(3-methoxycyclobutyl)-8-(1-methyl-1H-pyrazol-4-yl)-3H-pyrrolo[2, 3-c]isoquinoline (Compound 23)

A mixture of 1-(3-methoxycyclobutyl)-8-(1-methyl-1H-pyrazol-4-yl)-3-tosyl-3H-pyrrolo[2,3-c]isoquinoline (100 mg, 0.206 mmol) and KOH (35 mg, 0.62 mmol) in THF (2 mL), MeOH (1 mL) and H₂O (0.5 mL) was stirred at 80° C. for 2 hours to give a yellow mixture. The reaction mixture was poured into brine (10 mL) and extracted with EtOAc (20 mL×4). The combined organic layer was dried over anhydrous Na₂SO₄, filtered and concentrated. The residue was purified by prep-HPLC (0.225% FA as an additive), then lyophilized to give Compound 23 (21.00 mg, yield: 31% for 3 steps) as a white solid.

¹H NMR (400 MHz, DMSO-d₆) δ 1.95-2.05 (1H, m), 2.53-2.63 (2H, m), 2.85-2.90 (1H, m), 3.15-3.25 (3H, m) 3.60-3.70 (1H, m), 3.95 (3H, s), 4.00-4.10 (1H, m), 7.25-7.43 (1H, m), 7.72 (1H, d, J=8.4 Hz), 8.03 (0.5H, s), 8.05-8.15 (2H, m), 8.27 (0.5H, s), 8.30-8.40 (1H, m), 8.80-8.90 (1H, m), 11.75 (1H, brs).

### Example 17. Preparation of Compounds 24, 25, and 26

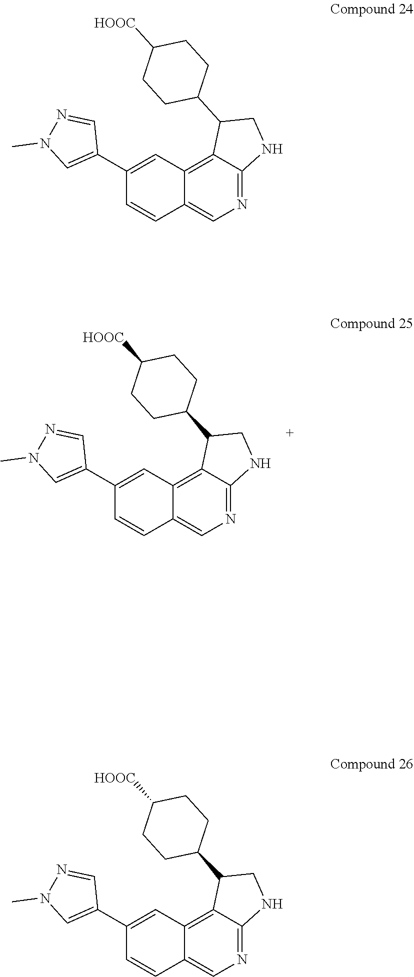 [SMILES: Cn1cc(-c2ccc3cnc4c(c3c2)C(C2CCC(C(=O)O)CC2)CN4)cn1 ; Cn1cc(-c2ccc3cnc4c(c3c2)C([C@H]2CC[C@@H](C(=O)O)CC2)CN4)cn1 ; Cn1cc(-c2ccc3cnc4c(c3c2)C([C@H]2CC[C@H](C(=O)O)CC2)CN4)cn1]

### Compound 24 Compound 25 Compound 26

### Step 1: Preparation of methyl 4-(8-(1-methyl-1H-pyrazol-4-yl)-2-(trimethylsilyl)-3H-pyrrolo[2,3-c]isoquinolin-1-yl)cyclohexane-1-carboxylate

A mixture of Int-5 (1.90 g, 5.43 mmol), Int-8 (3.23 g, 13.6 mmol), Pd(dppf)Cl₂ (397 mg, 0.542 mmol), KOAc (1.07 g, 10.9 mmol) and LiCl (230 mg, 5.43 mmol) in anhydrous DMF (40 mL) was degassed and purged with N₂ for 3 times. Then the mixture was stirred at 110° C. for 16 hours under N₂ atmosphere. The reaction mixture was concentrated and the residue was purified by flash silica gel chromatography (ISCO®; 20 g SepaFlash® Silica Flash Column, Eluent of 0˜₉₀% Ethyl acetate/Petroleum ether gradient @35 mL/min) to give methyl 4-(8-(1-methyl-1H-pyrazol-4-yl)-2-(trimethylsilyl)-3H-pyrrolo[2,3-c]isoquinolin-1-yl)cyclohexane-1-carboxylate (1.30 g, yield: 47%) as a yellow solid.

### Step 2: Preparation of methyl 4-(8-(1-methyl-1H-pyrazol-4-yl)-3H-pyrrolo[2,3-c]isoquinolin-1-yl)cyclohexane-1-carboxylate

To a solution of methyl 4-(8-(1-methyl-1H-pyrazol-4-yl)-2-(trimethylsilyl)-3H-pyrrolo[2,3-c]isoquinolin-1-yl)cyclohexane-1-carboxylate (1.30 g, 2.82 mmol) in THF (20 mL) was added TBAF (5.64 mL, 5.64 mmol, 1M in THF), then the mixture was stirred at 20° C. for 12 hours. The reaction mixture was poured into EtOAc (60 mL) and washed with saturated aqueous NH₄Cl (60 mL×2), dried over anhydrous Na₂SO₄, filtered and concentrated.

The residue was purified by Combi Flash (ISCO®; 12 g SepaFlash® Silica Flash Column, Eluent of 0˜3% MeOH/DCM gradient @30 mL/min) to give methyl 4-(8-(1-methyl-1H-pyrazol-4-yl)-3H-pyrrolo[2,3-c]isoquinolin-1-yl)cyclohexane-1-carboxylate (900 mg, yield: 79%) as a yellow solid.

¹H NMR (400 MHz, CDCl₃) δ 1.50-1.60 (1H, m), 1.75-1.95 (3H, m), 2.18-2.31 (2H, m), 2.34-2.57 (3H, m), 3.23-3.41 (1H, m), 3.72-3.77 (3H, m), 4.01-4.07 (3H, m), 7.10-7.17 (1H, m), 7.56-7.65 (1H, m), 7.77 (1H, s), 7.94 (1H, s), 8.04 (1H, d, J=8.4 Hz), 8.33 (1H, d, J=8.8 Hz), 8.84 (1H, d, J=2.4 Hz), 9.26-9.41 (1H, m).

### Step 3: Preparation of 4-(8-(1-methyl-1H-pyrazol-4-yl)-3H-pyrrolo[2, 3-c]isoquinolin-1-yl)cyclohexane-1-carboxylic acid (Compound 24)

A solution of methyl 4-(8-(1-methyl-1H-pyrazol-4-yl)-3H-pyrrolo[2,3-c]isoquinolin-1-yl)cyclohexane-1-carboxylate (800 mg, 2.06 mmol) and LiOH·H₂O (259 mg, 6.18 mmol) in H₂O (5 mL), THF (5 mL) and MeOH (5 mL) was stirred at 60° C. for 2 hours. The reaction mixture was concentrated to give Compound 24 (900 mg, crude, Li salt) as yellow solid, which was used into the next step without further purification.

### Step 4: Preparative HPLC purification

100 mg of Compound 24 (Li salt, crude) was poured into H₂O (15 mL) and acidified with 1M aqueous HCl to pH=6 and extracted with EtOAc (20 mL×5). The combined organic layer was concentrated and the residue was purified by prep-HPLC (0.225% FA as an additive), then lyophilized to give pure Compound 24 (25.85 mg, yield: 27%) as a light yellow solid.

¹H NMR (400 MHz, DMSO-d₆) δ 1.43-1.56 (1H, m), 1.57-1.68 (1H, m), 1.68-1.81 (1H, m), 1.81-1.95 (1H, m), 2.01-2.13 (2H, m), 2.15-2.34 (2.5H, m), 2.69-2.77 (0.5H, m), 3.27-3.30 (1H, m), 3.93 (3H, s), 7.15-7.20 (1H, m), 7.66-7.74 (1H, m), 8.00 (1H, d, J=8.8 Hz), 8.11 (1H, d, J=8.8 Hz), 8.27 (1H, s), 8.30 (1H, s), 8.75-8.82 (1H, m), 11.70-11.75 (1H, m).

### Step 5: SFC Purification

Compound 24 (100 mg, 0.263 mmol) was submitted for SFC separation (0.1% NH₃H₂O as an additive), then lyophilized to give Compound 25 (13.45 mg, yield: 14%) as a yellow solid and Compound 26 (26.71 mg, yield: 27%) as a light yellow solid.

Compound 25: ¹H NMR (400 MHz, DMSO-d₆) δ 1.10-1.30 (1H, m), 1.55-1.70 (2H, m), 1.80-1.95 (2H, m), 2.00-2.10 (2H, m), 2.20-2.30 (2H, m), 2.65-2.75 (1H, m), 3.94 (3H, s), 7.18 (1H, d, J=2.4 Hz), 7.71 (1H, d, J=8.4 Hz), 8.02 (1H, s), 8.11 (1H, d, J=8.4 Hz), 8.28 (1H, s), 8.31 (1H, s), 8.79 (1H, s), 11.72 (1H, d, J=1.6 Hz).

Compound 26: ¹H NMR (400 MHz, DMSO-d₆) δ 1.45-1.60 (2H, m), 1.65-1.80 (2H, m), 2.05-2.15 (2H, m), 2.20-2.35 (3H, m), 3.23-3.26 (1H, m), 3.93 (3H, s), 7.19 (1H, d, J=2.0 Hz), 7.69 (1H, dd, J=8.4, 1.2 Hz), 7.99 (1H, s), 8.11 (1H, d, J=8.4 Hz), 8.26 (1H, s), 8.30 (1H, s), 8.79 (1H, s), 11.73 (1H, brs).

The following compounds were synthesized analogously to Compounds 24, 25, and 26

| Comp. | | | |
|---|---|---|---|
| No | Structure | Name | ¹H NMR (400 MHz) |
| 32 | 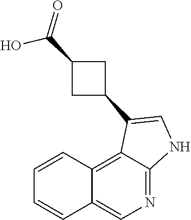 [SMILES: O=C(O)[C@H]1C[C@@H](c2c[nH]c3ncc4ccccc4c32)C1] | (1s,3s)-3-(3H- pyrrolo[2,3- c]isoquinolin-1- yl)cyclobutane-1- carboxylic acid | CD₃OD; δ 2.40-2.55 (2H, m), 2.90-3.00 (2H, m), 3.35-3.40 (1H, m), 4.10-4.23 (1H, m), 7.62 (1H, s), 7.75-7.85 (1H, m), 8.15-8.20 (1H, m), 8.42 (1H, d, J = 8.0 Hz), 8.54 (1H, d, J = 8.4 Hz), 9.26 (1H, s). |
| 33 | 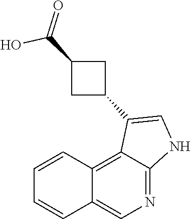 [SMILES: O=C(O)[C@H]1C[C@H](c2c[nH]c3ncc4ccccc4c32)C1] | (1r,3r)-3-(3H- pyrrolo[2,3- c]isoquinolin-1- yl)cyclobutane-1- carboxylic acid | CD₃OD; δ 2.50-2.65 (2H, m), 2.90-3.05 (2H, m), 3.20-3.30 (1H, m), 4.20-4.35 (1H, m), 7.70 (1H, s), 7.75-7.85 (1H, m), 8.10-8.20 (1H, m), 8.35- 8.50 (2H, m), 9.25 (1H, s). |
| 34 | 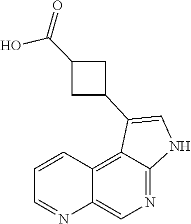 [SMILES: O=C(O)C1CC(c2c[nH]c3ncc4ncccc4c23)C1] | 3-(3H-pyrrolo[3,2- f][1,7]naphthyridin- 1-yl)cyclobutane-1- carboxylic acid | DMSO-d₆; δ 2.22-2.35 (2H, m), 2.70-2.84 (2H, m), 3.12- 3.24 (1H, m), 3.89-4.03 (1H, m), 7.50-7.70 (1H, m), 7.84- 7.93 (1H, m), 8.50-8.80 (1H, m), 8.90-9.00 (1H, m), 9.04 (1H, s), 12.23 (1H, brs). |
| 35 | 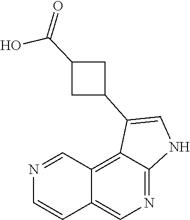 [SMILES: O=C(O)C1CC(c2c[nH]c3ncc4ccncc4c23)C1] | 3-(7H-pyrrolo[2,3- c][2,6]naphthyridin- 9-yl)cyclobutane-1- carboxylic acid | DMSO-d₆; δ 2.27-2.37 (2H, m), 2.72-2.84 (2H, m), 3.10- 3.24 (1H, m), 3.98-4.18 (1H, m), 7.50-7.70 (1H, m), 8.05 (1H, d, J = 4.4 Hz), 8.61 (1H, d, J = 5.6 Hz), 8.90-9.10 (1H, m), 9.50-9.70 (1H, m), 12.20 (1H, brs). |
| 36 | 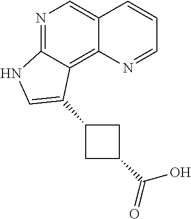 [SMILES: O=C(O)[C@H]1C[C@@H](c2c[nH]c3ncc4cccnc4c32)C1] | (1s,3s)-3-(7H- pyrrolo[2,3- h][1,6]naphthyridin- 9-yl)cyclobutane-1- carboxylic acid | DMSO-d₆; δ 2.25-2.45 (2H, m), 2.60-2.70 (2H, m), 2.99- 3.24 (1H, m), 4.15-4.35 (1H, m), 7.40 (1H, s), 7.52 (1H, dd, J = 8.0, 4.4 Hz), 8.50-8.60 (1H, m), 8.92 (1H, s), 9.06 (1H, d, J = 4.4, 2.0 Hz), 12.00 (1H, brs). |
| 37 | 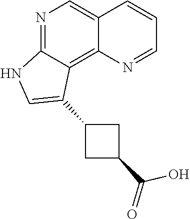 [SMILES: O=C(O)[C@H]1C[C@H](c2c[nH]c3ncc4cccnc4c32)C1] | (1r,3r)-3-(7H- pyrrolo[2,3- h][1,6]naphthyridin- 9-yl)cyclobutane-1- carboxylic acid | DMSO-d₆; δ 2.43-2.47 (2H, m), 2.63-2.72 (2H, m), 3.10- 3.20 (1H, m), 4.25-4.40 (1H, m), 7.40-7.60 (2H, m), 8.50- 8.55 (1H, m), 8.92 (1H, s), 9.08 (1H, dd, J = 4.4, 2.4 Hz), 12.01 (1H, brs). |

### Example 18. Preparation of Compound 27

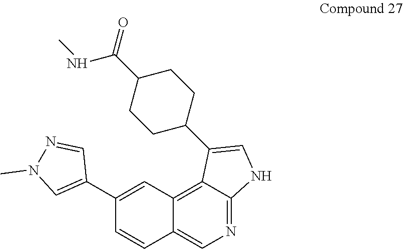 [SMILES: CNC(=O)C1CCC(c2c[nH]c3ncc4ccc(-c5cnn(C)c5)cc4c23)CC1]

A solution of Compound 24 (100 mg, 0.263 mmol), methylamine hydrochloride (53 mg, 0.79 mmol), EDCI (126 mg, 0.657 mmol), HOBt (89 mg, 0.66 mmol) and DIPEA (204 mg, 1.58 mmol) in anhydrous DMF (5 mL) was stirred at 45° C. for 12 hours. The reaction mixture was poured into water (30 mL) and extracted with EtOAc (30 mL×3). The combined organic layer was dried over anhydrous Na₂SO₄, filtered and concentrated. The residue was purified by prep-HPLC (0.225% FA as an additive), then lyophilized to give Compound 27 (45.34 mg, yield: 51%) as a yellow solid.

¹H NMR (400 MHz, DMSO-d₆) δ 1.40-2.10 (7H, m), 2.20-2.35 (2H, m), 2.56-2.65 (3H, m), 3.20-3.30 (1H, m), 3.94 (3H, s), 7.16-7.23 (1H, m), 7.62-7.78 (2H, m), 7.94-8.03 (1H, m), 8.07-8.14 (1H, m), 8.22-8.34 (2H, m), 8.76-8.82 (1H, m), 11.67-11.77 (1H, m).

### Example 19. Preparation of Compound 28

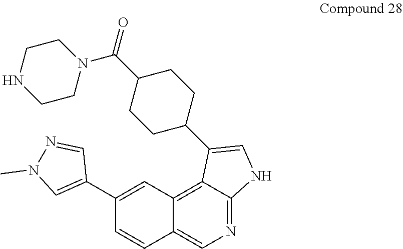 [SMILES: Cn1cc(-c2ccc3cnc4[nH]cc(C5CCC(C(=O)N6CCNCC6)CC5)c4c3c2)cn1]

### Step 1: Preparation of tert-butyl 4-(4-(8-(1-methyl-1H-pyrazol-4-yl)-3H-pyrrolo[2,3-c]isoquinolin-1-yl)cyclohexane-1-carbonyl)piperazine-1-carboxylate

A mixture of Compound 24 (200 mg, 0.526 mmol), 1-Boc-piperazine (196 mg, 1.05 mmol), EDCI (252 mg, 1.31 mmol), HOBt (178 mg, 1.31 mmol) and DIPEA (408 mg, 3.15 mmol) in anhydrous DMF (4 mL) was stirred at 45° C. for 12 hours. The reaction mixture was poured into water (40 mL) and extracted with DCM (40 mL×3). The combined organic layer was dried over anhydrous Na₂SO₄, filtered and concentrated. The residue was purified by Combi Flash (ISCO®; 4 g SepaFlash® Silica Flash Column, Eluent of 0˜7% MeOH/DCM gradient @20 mL/min) tert-butyl 4-(4-(8-(1-methyl-1H-pyrazol-4-yl)-3H-pyrrolo[2,3-c]isoquinolin-1-yl)cyclohexane-1-carbonyl)piperazine-1-carboxylate (230 mg, yield: 88%) as a light yellow solid.

### Step 2: Preparation of (4-(8-(1-methyl-1H-pyrazol-4-yl)-3H-pyrrolo[2,3-c]isoquinolin-1-yl)cyclohexyl)(piperazin-1-yl)methanone (Compound 28)

To a solution of tert-butyl 4-(4-(8-(1-methyl-1H-pyrazol-4-yl)-3H-pyrrolo[2,3-c]isoquinolin-1-yl)cyclohexane-1-carbonyl)piperazine-1-carboxylate (230 mg, 0.424 mmol) in MeOH (5 mL) was added 4M HCl/MeOH (10 mL) at 0° C., the mixture was stirred at 20° C. for 2 hours. The reaction mixture was concentrated and the residue was purified by prep-HPLC (0.05% NH₃H₂O as an additive), then lyophilized to give Compound 28 (116.70 mg, yield: 61%) as a white solid.

¹H NMR (400 MHz, DMSO-d₆) δ 1.49-1.65 (1.3H, m), 1.70-1.89 (4H, m), 1.91-2.09 (1.6H, m), 2.18-2.29 (1.3H, m), 2.58-2.73 (4.7H, m), 2.88-2.97 (0.4H, m), 3.39-3.49 (4.7H, m), 3.93 (3H, s), 7.16-7.22 (1H, m), 7.66-7.73 (1H, m), 7.93-8.02 (1H, m), 8.07-8.15 (1H, m), 8.23-8.34 (2H, m), 8.75-8.82 (1H, m), 11.67-11.76 (1H, m).

### Example 20. Preparation of Compound 29

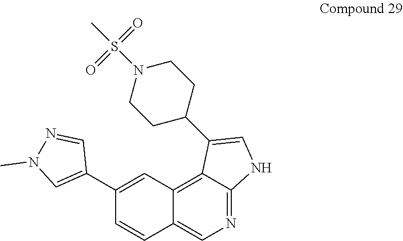 [SMILES: Cn1cc(-c2ccc3cnc4[nH]cc(C5CCN(S(C)(=O)=O)CC5)c4c3c2)cn1]

### Step 1: Preparation of tert-butyl 4-(8-(1-methyl-1H-pyrazol-4-yl)-2-(trimethylsilyl)-3H-pyrrolo[2,3-c]isoquinolin-1-yl)piperidine-1-carboxylate

A mixture of Int-5 (400 mg, 1.14 mmol), Int-9 (643 mg, 2.28 mmol), Pd(dppf)Cl₂ (84 mg, 0.11 mmol), KOAc (224 mg, 2.28 mmol) and LiCl (48 mg, 1.1 mmol) in anhydrous DMF (10 mL) was degassed and purged with N₂ for 3 times and then the mixture was stirred at 110° C. for 17 hours under N₂ atmosphere. The reaction mixture was concentrated and the residue was purified by Combi Flash (ISCO®; 4 g SepaFlash® Silica Flash Column, Eluent of 0-60% Ethyl acetate/Petroleum ether gradient @40 mL/min) to give tert-butyl 4-(8-(1-methyl-TH-pyrazol-4-yl)-2-(trimethylsilyl)-3H-pyrrolo[2,3-c]isoquinolin-1-yl)piperidine-1-carboxylate (440 mg, yield: 70%) as a light yellow solid.

### Step 2: Preparation of tert-butyl 4-(8-(1-methyl-1H-pyrazol-4-yl)-3H-pyrrolo[2,3-c]isoquinolin-1-yl)piperidine-1-carboxylate

To a solution of tert-butyl 4-(8-(1-methyl-TH-pyrazol-4-yl)-2-(trimethylsilyl)-3H-pyrrolo[2,3-c]isoquinolin-1-yl)piperidine-1-carboxylate (440 mg, 0.812 mmol) in THF (10 mL) was added TBAF (2.44 mL, 2.44 mmol, 1M in THF) and the mixture was stirred at 20° C. for 12 hours. The reaction mixture was diluted with EtOAc (40 mL) and washed with saturated aqueous NH₄Cl (40 mL×2), dried over anhydrous Na₂SO₄, filtered and concentrated. The residue was purified by Combi Flash (ISCO®; 12 g SepaFlash® Silica Flash Column, Eluent of 0˜80% Ethyl acetate/Petroleum ether gradient @25 mL/min) to give tert-butyl 4-(8-(1-methyl-TH-pyrazol-4-yl)-3H-pyrrolo[2,3-c]isoquinolin-1-yl)piperidine-1-carboxylate (240 mg, yield: 69%) as a light yellow solid.

¹H NMR (400 MHz, CDCl₃) δ 1.52 (9H, s), 1.65-1.80 (2H, m), 2.25-2.35 (2H, m), 2.95-3.15 (2H, m), 3.35-3.50 (1H, m), 4.04 (3H, s), 4.25-4.50 (2H, m), 7.11 (1H, d, J=2.4 Hz), 7.62 (1H, d, J=8.4), 7.76 (1H, s), 7.91 (1H, s), 8.05 (1H, d, J=8.4 Hz), 8.32 (1H, s), 8.82 (1H, brs), 8.85 (1H, s).

### Step 3: Preparation of 8-(1-methyl-1H-pyrazol-4-yl)-(piperidin-4-yl)-3H-pyrrolo[2,3-c]isoquinoline

To a solution of tert-butyl 4-(8-(1-methyl-1H-pyrazol-4-yl)-3H-pyrrolo[2,3-c]isoquinolin-1-yl)piperidine-1-carboxylate (200 mg, 0.463 mmol) in EtOAc (10 mL) was added 4M HCl/EtOAc (8.33 mL, 33.3 mmol) at 0° C., the mixture was stirred at 0° C. for 0.5 hour and then at 20° C. for 0.5 hour. The reaction mixture was concentrated and the residue was diluted with water (40 mL) and basified with 2M NaOH to pH=12 and extracted with DCM (30 mL×5). The combined organic layer was dried over anhydrous Na₂SO₄, filtered and concentrated give 8-(1-methyl-TH-pyrazol-4-yl)-1-(piperidin-4-yl)-3H-pyrrolo[2,3-c]isoquinoline (150 mg, crude) as a light yellow solid, which was directly used for the next step without purification.

¹H NMR (400 MHz, CD₃OD) δ 1.65-1.80 (2H, m), 2.20-2.30 (2H, m), 2.90-3.05 (2H, m), 3.20-3.30 (2H, m), 3.40-3.50 (1H, m), 3.99 (3H, s), 7.20 (1H, s), 7.68 (1H, dd, J=8.4, 1.6 Hz), 7.95 (1H, s), 8.07 (1H, d, J=8.4 Hz), 8.11 (1H, s), 8.35 (1H, s), 8.73 (1H, s).

### Step 4: Preparation of 8-(1-methyl-1H-pyrazol-4-yl)-1-(1-(methylsulfonyl)piperidin-4-yl)-3H-pyrrolo[2, 3-c]isoquinoline (Compound 29)

To a solution of 8-(1-methyl-1H-pyrazol-4-yl)-1-(piperidin-4-yl)-3H-pyrrolo[2,3-c]isoquinoline (130 mg, 0.392 mmol) and TEA (119 mg, 1.18 mmol) in anhydrous DCM (1 mL) was added a solution of MsCl (180 mg, 1.57 mmol) in DCM (1 mL) dropwise at 0° C., the mixture was stirred at 0° C. for 1 hour and then at 20° C. for 11 hours. The reaction mixture was quenched with saturated aqueous NaHCO₃ (20 mL) and water (30 mL), then extracted with DCM (25 mL×3). The combined organic layer was washed with brine (40 mL), dried over anhydrous Na₂SO₄, filtered and concentrated. The residue was purified by prep-HPLC (0.04% HCl as an additive) to give Compound 29 (23.26 mg, yield: 13% for 2 steps, HCl salt) as a yellow solid.

¹H NMR (400 MHz, DMSO-d₆) δ 1.65-1.85 (2H, m), 2.15-2.30 (2H, m), 2.98 (3H, s), 3.10-3.20 (2H, m), 3.40-3.55 (1H, m), 3.75-3.80 (2H, m), 3.94 (3H, s), 7.34 (1H, s), 7.78 (1H, d, J=8.8 Hz), 8.10 (1H, s), 8.18 (1H, d, J=8.4 Hz), 8.29 (1H, s), 8.36 (1H, s), 8.90 (1H, s), 12.00 (1H, brs).

### Example 21. Preparation of Compound 30

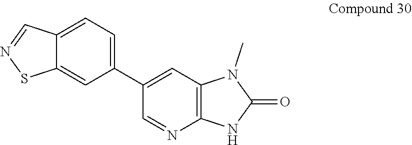 [SMILES: Cn1c(=O)[nH]c2ncc(-c3ccc4cnsc4c3)cc21]

### Step 1: Preparation of benzyl (2-amino-5-bromopyridin-3-yl)carbamate

To a solution of 5-bromopyridine-2,3-diamine (2.50 g, 13.3 mmol), pyridine (3.16 g, 39.9 mmol) in THF (50 mL) was added benzyl carbonochloridate (2.27 g, 13.3 mmol) at 0° C.

The mixture was stirred at 25° C. for 3 hours. The reaction mixture was concentrated and the residue was diluted with H₂O (50 mL), then extracted with EtOAc (80 mL×3). The combined organic layers were washed with saturated aqueous NaHCO₃ (80 mL), brine (100 mL), dried over anhydrous Na₂SO₄, filtered and concentrated. The residue was purified by silica gel column (0% to 40% EtOAc in PE) to give benzyl (2-amino-5-bromopyridin-3-yl)carbamate (2.50 g, yield: 49%) as a white solid.

### Step 2: Preparation of 5-bromo-N³-methylpyridine-2, 3-diamine

To a suspension of LiAlH₄ (848 mg, 22.3 mmol) in Et₂₀ (50 mL) was added benzyl (2-amino-5-bromopyridin-3-yl)carbamate (1.80 g, 5.59 mmol) at 0° C. The mixture was stirred at 25° C. for 5 hours. The reaction mixture was quenched by with H₂O (1 mL) and 1N aqueous NaOH (1 mL), then diluted with H₂O (50 mL) and extracted with EtOAc (80 mL×3). The combined organic layers were washed with brine (100 mL), dried over anhydrous Na₂SO₄, filtered and concentrated. The residue was purified by silica gel column (0% to 80% EtOAc in PE) to give 5-bromo-N³-methylpyridine-2,3-diamine (1.00 g, yield: 70%) as a white solid.

¹H NMR (400 MHz, DMSO-d₆) δ 2.70 (3H, d, J=4.8 Hz), 5.16-5.29 (1H, m), 5.66 (2H, brs), 6.57 (1H, d, J=2.0 Hz), 7.29 (1H, d, J=2.0 Hz).

### Step 3: Preparation of 6-bromo-1-methyl-1,3-dihydro-2H-imidazo[4,5-b]pyridin-2-one

To a solution of 5-bromo-N³-methylpyridine-2,3-diamine (800 mg, 3.96 mmol) in CH₃CN (20 mL) was added CDI (1.28 g, 7.92 mmol). The mixture was stirred at 80° C. for 3 hours. The reaction mixture was concentrated and the residue was purified by silica gel column (0% to 90% EtOAc in PE) to give 6-bromo-1-methyl-1,3-dihydro-2H-imidazo[4,5-b]pyridin-2-one (900 mg, yield: 89%) as a white solid.

¹H NMR (400 MHz, DMSO-d₆) δ 3.28 (3H, s), 7.74 (1H, d, J=2.0 Hz), 7.99 (1H, d, J=2.0 Hz), 11.74 (1H, brs).

### Step 4: Preparation of 6-bromo-1-methyl-3-trityl-1,3-dihydro-2H-imidazo[4,5-b]pyridin-2-one

To a solution of 6-bromo-1-methyl-1,3-dihydro-2H-imidazo[4,5-b]pyridin-2-one (100 mg, 0.438 mmol) in DCM (2 mL) was added Trt-C1 (141 mg, 0.504 mmol) and Et₃N (73 mg, 0.72 mmol). The mixture was stirred at 25° C. for 16 hours. The reaction mixture was diluted with H₂O (10 mL) and extracted with DCM (20 mL×3). The combined organic layers were washed with brine (30 mL), dried over anhydrous Na₂SO₄, filtered and concentrated. The residue was purified by silica gel column (0% to 30% EtOAc in PE) to give 6-bromo-1-methyl-3-trityl-1,3-dihydro-2H-imidazo[4,5-b]pyridin-2-one (250 mg, yield: 61%) as a white solid.

### Step 5: Preparation of 6-(benzo[d]isothiazol-6-yl)-1-methyl-3-trityl-1,3-dihydro-2H-imidazo[4,5-b]pyridin-2-one

To a solution of 6-bromo-1-methyl-3-trityl-1,3-dihydro-2H-imidazo[4,5-b]pyridin-2-one (200 mg, 0.425 mmol), Int-10 (122 mg, 0.468 mmol) in dioxane (4 mL) and H₂O (0.5 mL) was added Pd(dppf)Cl₂ (31 mg, 0.042 mmol) and Na₂CO₃ (135 mg, 1.28 mmol), then the mixture was degassed and purged with N₂ for 3 times and the mixture was stirred at 90° C. for 12 hours under N₂ atmosphere. The reaction mixture was suspended in CH₃OH (20 mL) and filtered. The filtrate was concentrated and the residue was purified by silica gel column (0% to 35% EtOAc in PE) to give 6-(benzo[d]isothiazol-6-yl)-1-methyl-3-trityl-1,3-dihydro-2H-imidazo[4,5-b]pyridin-2-one (170 mg, yield: 76%) as a yellow solid.

### Step 6: Preparation of 6-(benzo[d]isothiazol-6-yl)-1-methyl-1,3-dihydro-2H-imidazo[4,5-b]pyridin-2-one (Compound 30)

To a solution of 6-(benzo[d]isothiazol-6-yl)-1-methyl-3-trityl-1,3-dihydro-2H-imidazo[4,5-b]pyridin-2-one (170 mg, 0.324 mmol) in DCM (5 mL) was added TFA (2 mL). The mixture was stirred at 25° C. for 1 hour. The reaction mixture was concentrated and the residue was triturated with MeOH (5 mL) to afford Compound 30 (29.61 mg, yield: 31%) as an off-white solid.

¹H NMR (400 MHz, DMSO-d₆) δ 3.38 (3H, s), 7.87-7.93 (2H, m), 8.31 (1H, d, J=8.4 Hz), 8.37 (1H, d, J=2.0 Hz), 8.57 (1H, s), 9.16 (1H, s), 11.69 (1H, brs).

### Example 22. Preparation of Compound 31

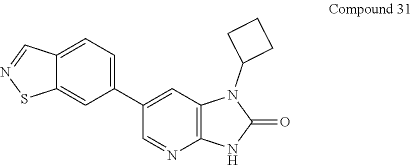 [SMILES: O=c1[nH]c2ncc(-c3ccc4cnsc4c3)cc2n1C1CCC1]

### Step 1: Preparation of 5-bromo-N³-cyclobutylpyridine-2, 3-diamine

To a solution of 5-bromopyridine-2,3-diamine (500 mg, 28.5 mmol) and cyclobutanone (186 mg, 28.5 mmol) in DCM (3 mL) was added AcOH (160 mg, 31.4 mmol) at 20° C. The reaction mixture was stirred at 20° C. for 5 hours. NaBH(OAc)₃ (845 mg, 42.8 mmol) was added and the reaction mixture was stirred at 20° C. for another 16 hours. The reaction mixture was basified with saturated aqueous NaHCO₃ to pH=8 and extracted with DCM (30 mL×3). The combined organic layer was washed with brine (30 mL), dried over anhydrous Na₂SO₄ and concentrated. The residue was purified by Combi Flash (SiO₂, 75% EtOAc in PE) to give 5-bromo-N³-cyclobutylpyridine-2, 3-diamine (397 mg, yield: 62%) as a brown solid.

¹H NMR (400 MHz, CDCl₃) δ 1.76-1.94 (4H, m), 2.36-2.55 (2H, m), 3.40-3.50 (1H, m), 3.80-3.90 (1H, m), 4.20 (2H, brs), 6.74 (1H, s), 7.60 (1H, d, J=2.0 Hz).

### Step 2: Preparation of 6-bromo-1-cyclobutyl-1,3-dihydro-2H-imidazo[4,5-b]pyridin-2-one

A solution of 5-bromo-N³-cyclobutylpyridine-2,3-diamine (1.14 g, 4.71 mmol) and CDI (1.53 g, 9.42 mmol) in CH₃CN (10 mL) was stirred at 80° C. for 5 hours. The reaction mixture was concentrated and the residue was purified by Combi Flash (SiO₂, 50% EtOAc in PE) to give 6-bromo-1-cyclobutyl-1,3-dihydro-2H-imidazo[4,5-b]pyridin-2-one (1.13 g, yield: 89%) as a yellow solid.

¹H NMR (400 MHz, DMSO-d₆) δ 1.63-1.92 (2H, m), 2.18-2.31 (2H, m), 2.68-2.83 (2H, m), 4.75-4.80 (1H, m), 7.87 (1H, s), 7.99 (1H, d, J=2.0 Hz), 11.76 (1H, brs).

### Step 3: Preparation of 6-bromo-1-cyclobutyl-3-trityl-1,3-dihydro-2H-imidazo[4,5-b]pyridin-2-one

To a solution of 6-bromo-1-cyclobutyl-1,3-dihydro-2H-imidazo[4,5-b]pyridin-2-one (553 mg, 2.07 mmol) and Et₃N (364 mg, 3.59 mmol) in DCM (8 mL) was added TrtCl (633 mg, 2.21 mmol) at 20° C. The reaction mixture was stirred at 20° C. for 16 hours. The product mixture was diluted with EtOAc (50 mL) and washed with saturated aqueous NaHCO₃ (30 mL×3), brine (30 mL), dried over anhydrous MgSO₄, filtered, and concentrated. The residue was purified by Combi Flash (SiO₂, 75% EtOAc in PE) to give 6-bromo-1-cyclobutyl-3-trityl-1,3-dihydro-2H-imidazo[4,5-b]pyridin-2-one (800 mg, yield: 76%) as colorless gum.

### Step 4: Preparation of 6-(benzo[d]isothiazol-6-yl)-1-cyclobutyl-3-trityl-1,3-dihydro-2H-imidazo[4,5-b]pyridin-2-one

A mixture of 6-bromo-1-cyclobutyl-3-trityl-1,3-dihydro-2H-imidazo[4,5-b]pyridin-2-one (200 mg, 0.391 mmol), 6-(4,4,5,5-tetramethyl-1,3,2-dioxaborolan-2-yl)benzo[d]isothiazole (112 mg, 0.431 mmol), Pd(dppf)Cl₂ (43 mg, 0.043 mmol) and Na₂CO₃ (125 mg, 1.25 mmol) in dioxane (5 mL) and H₂O (0.5 mL) was degassed and purged with N₂ for 3 times, then the mixture was stirred at 90° C. for 3 hours under N₂ atmosphere. The reaction mixture was diluted with water (20 mL) and EtOAc (30 mL), then filtered through a pad of celite. The filtrate was separated and the aqueous layer was extracted with EtOAc (30 mL×2). The combined organic layer was washed with brine (30 mL), dried over anhydrous Na₂SO₄, filtered and concentrated. The residue was purified by Combi Flash (SiO₂, 75% EtOAc in PE) to give 6-(benzo[d]isothiazol-6-yl)-1-cyclobutyl-3-trityl-1,3-dihydro-2H-imidazo[4,5-b]pyridin-2-one (170 mg, yield: 77%) as a yellow solid.

¹H NMR (400 MHz, DMSO-d₆) δ 1.73-1.99 (2H, m), 2.27-2.41 (2H, m), 2.79-2.95 (2H, m), 4.80-4.99 (1H, m), 7.17-7.25 (3H, m), 7.27-7.35 (6H, m), 7.54-7.60 (6H, m), 7.91 (1H, dd, J=8.4, 1.2 Hz), 8.02 (1H, d, J=2.0 Hz), 8.23 (1H, s), 8.31 (1H, d, J=8.4 Hz), 8.61 (1H, s), 9.19 (1H, s).

### Step 5: Preparation of 6-(benzo[d]isothiazol-6-yl)-1-cyclobutyl-1,3-dihydro-2H-imidazo[4,5-b]pyridin-2-one (Compound 31)

A solution of 6-(benzo[d]isothiazol-6-yl)-1-cyclobutyl-3-trityl-1,3-dihydro-2H-imidazo[4,5-b]pyridin-2-one (170 mg, 0.301 mmol) in DCM (5 mL) and TFA (5 mL) was stirred at 20° C. for 3 hours under N₂ atmosphere. The reaction mixture was concentrated and the crude product was triturated with CH₃CN (10 mL) to give Compound 31 (18 mg, yield: 18%) as a yellow solid.

¹H NMR (400 MHz, DMSO-d₆) δ 1.71-1.97 (2H, m), 2.25-2.37 (2H, m), 2.80-2.90 (2H, m), 4.80-4.90 (1H, m), 7.90 (1H, dd, J=8.4, 1.6 Hz), 8.00 (1H, s), 8.31 (1H, d, J=8.4 Hz), 8.36 (1H, d, J=2.0 Hz), 8.59 (1H, s), 9.16 (1H, s), 11.71 (1H, brs)

### Example 23. Preparation of Compound 38

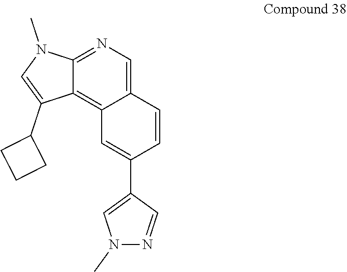 [SMILES: Cn1cc(-c2ccc3cnc4c(c(C5CCC5)cn4C)c3c2)cn1]

### Step 1: Preparation of 1-cyclobutyl-8-(1-methyl-1H-pyrazol-4-yl)-2-(trimethylsilyl)-3H-pyrrolo[2,3-c]isoquinoline

A mixture of Int-5 (300 mg, 0.857 mmol), Int-26 (261 mg, 1.71 mmol), Pd(dppf)Cl₂ (63 mg, 0.086 mmol), LiCl (36 mg, 0.86 mmol) and Na₂CO₃ (272 mg, 2.57 mmol) in anhydrous DMF (5 mL) was degassed and purged with N₂ for 3 times, and then the mixture was stirred at 105° C. for 16 hours under N₂ atmosphere. The reaction mixture was concentrated and the residue was purified by Combi Flash (0% to 60% EtOAc in PE) to give 1-cyclobutyl-8-(1-methyl-1H-pyrazol-4-yl)-2-(trimethylsilyl)-3H-pyrrolo[2,3-c]isoquinoline (220 mg, yield: 69%) as a yellow solid.

¹H NMR (400 MHz, DMSO-d₆) δ 0.48 (9H, s), 2.20-2.30 (2H, m), 2.40-2.50 (2H, m), 2.70-2.80 (2H, m), 4.00 (3H, s), 4.25-4.35 (1H, m), 7.78 (1H, d, J=8.40), 8.01 (1H, s), 8.20 (1H, d, J=8.40 Hz), 8.32 (1H, s), 8.64 (1H, s), 8.89 (1H, s).

### Step 2: Preparation of 1-cyclobutyl-8-(1-methyl-1H-pyrazol-4-yl)-3H-pyrrolo[2,3-c]isoquinoline

A mixture of 1-cyclobutyl-8-(1-methyl-1H-pyrazol-4-yl)-2-(trimethylsilyl)-3H-pyrrolo[2,3-c]isoquinoline (220 mg, 0.587 mmol) in TFA (4 mL) was stirred at 50° C. for 2 hours. The reaction mixture was concentrated and the residue was basified with saturated aqueous NaHCO₃ to pH=8 and extracted with EtOAc (50 mL×2). The combined organic layer was dried over anhydrous Na₂SO₄, filtered and concentrated. The residue was purified by Combi Flash (0% to 80% EtOAc in PE) to give 1-cyclobutyl-8-(1-methyl-1H-pyrazol-4-yl)-3H-pyrrolo[2,3-c]isoquinoline (130 mg, yield: 73%) as a yellow solid.

¹H NMR (400 MHz, DMSO-d₆) δ 1.91-1.98 (1H, m), 2.11-2.25 (3H, m), 2.56-2.63 (2H, m), 3.95 (3H, s), 4.09-4.22 (1H, m), 7.31 (1H, d, J=1.6 Hz), 7.71 (1H, dd, J=8.4, 1.2 Hz), 8.04 (1H, s), 8.10 (1H, d, J=8.80 Hz), 8.22 (1H, s), 8.35 (1H, s), 8.79 (1H, s), 11.73 (1H, brs).

### Step 3: Preparation of 1-cyclobutyl-3-methyl-8-(1-methyl-1H-pyrazol-4-yl)-3H-pyrrolo[2,3-c]isoquinoline

To a solution of 1-cyclobutyl-8-(1-methyl-1H-pyrazol-4-yl)-3H-pyrrolo[2,3-c]isoquinoline (65 mg, 0.21 mmol) in anhydrous DMF (3 mL) was added NaH (17 mg, 0.43 mmol, 60% dispersion in mineral oil) at 0° C. Then MeI (92 mg, 0.64 mmol) was added to the reaction mixture at 0° C. The mixture was stirred at 0° C. for 1 hour, then quenched with water (5 mL) at 0° C. and extracted with EtOAc (25 mL×2). The combined organic layer was dried over anhydrous Na₂SO₄, filtered and concentrated. The residue was purified by prep-HPLC (0.225% FA as an additive) and lyophilized to give Compound 38 (13.2 mg, yield: 19%) as an off-white solid.

¹H NMR (400 MHz, DMSO-d₆) δ 1.92-2.01 (1H, m), 2.10-2.23 (3H, m), 2.56-2.64 (2H, m), 3.92 (3H, s), 3.94 (3H, s), 4.10-4.22 (1H, m), 7.40 (1H, s), 7.72 (1H, d, J=8.0 Hz), 8.05 (1H, s), 8.12 (1H, d, J=8.4 Hz), 8.21 (1H, s), 8.36 (1H, s), 8.84 (1H, s).

### Example 24. Preparation of Compound 39 and 40

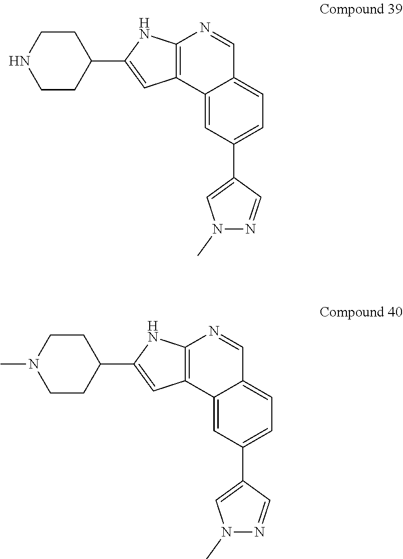 [SMILES: Cn1cc(-c2ccc3cnc4[nH]c(C5CCNCC5)cc4c3c2)cn1 ; CN1CCC(c2cc3c(ncc4ccc(-c5cnn(C)c5)cc43)[nH]2)CC1]

### Step 1: Preparation of tert-butyl 4-((3-amino-6-(1-methyl-1H-pyrazol-4-yl)isoquinolin-4-yl)ethynyl)piperidine-1-carboxylate

A mixture of Int-5 (300 mg, 857 mmol), tert-butyl 4-ethynylpiperidine-1-carboxylate (538 mg, 2.57 mmol), CuI (33 mg, 0.17 mmol), Pd(PPh₃)₂Cl₂ (60 mg, 0.086 mmol) and Et₃N (433 mg, 4.28 mmol) in anhydrous DMF (10 mL) was degassed and purged with N₂ for 3 times. Then the mixture was stirred at 60° C. for 4 hours under N₂ atmosphere. The reaction mixture was concentrated. The residue was purified by Combi Flash (0% to 80% EtOAc in PE) to give tert-butyl 4-((3-amino-6-(1-methyl-1H-pyrazol-4-yl)isoquinolin-4-yl)ethynyl)piperidine-1-carboxylate (360 mg, yield: 97%) as a yellow solid.

### Step 2: Preparation of tert-butyl 4-(8-(1-methyl-1H-pyrazol-4-yl)-3H-pyrrolo[2,3-c]isoquinolin-2-yl)piperidine-1-carboxylate

A mixture of tert-butyl 4-((3-amino-6-(1-methyl-1H-pyrazol-4-yl)isoquinolin-4-yl)ethynyl)piperidine-1-carboxylate (200 mg, 0.463 mmol) and t-BuOK (208 mg, 1.85 mmol) in DMF (5 mL) was stirred at 80° C. for 2 hours. The reaction mixture was concentrated and the residue was purified by Combi Flash (0% to 90% EtOAc in PE) to give tert-butyl 4-(8-(1-methyl-1H-pyrazol-4-yl)-3H-pyrrolo[2,3-c]isoquinolin-2-yl)piperidine-1-carboxylate (150 mg, yield: 75%) as a yellow solid.

¹H NMR (400 MHz, DMSO-d₆) δ 1.40 (9H, s), 1.50-1.70 (2H, m), 2.00-2.10 (2H, m), 2.90-3.00 (2H, m), 3.25-3.35 (2H, m), 3.91 (3H, s), 4.05-4.15 (1H, m), 6.81 (1H, s), 7.68 (1H, d, J=8.4 Hz), 7.95 (1H, s), 8.04 (1H, d, J=8.4 Hz), 8.30-8.40 (2H, m), 8.72 (1H, s), 11.80 (1H, brs).

### Step 3: Preparation of 8-(1-methyl-1H-pyrazol-4-yl)-2-(piperidin-4-yl)-3H-pyrrolo[2, 3-c]isoquinoline (Compound 39)

A mixture of tert-butyl 4-(8-(1-methyl-1H-pyrazol-4-yl)-3H-pyrrolo[2,3-c]isoquinolin-2-yl)piperidine-1-carboxylate (40 mg, 0.093 mmol) in TFA (1 mL) and DCM (4 mL) was stirred at 25° C. for 4 hours. The reaction mixture was concentrated and the residue was purified by prep-HPLC (0.1% TFA as an additive), then lyophilized to give Compound 39 (7.78 mg, yield: 25%, TFA salt) as a yellow solid.

¹H NMR (400 MHz, CD₃OD) δ 2.00-2.17 (2H, m), 2.40-2.50 (2H, m), 3.20-3.30 (3H, m), 3.55-3.65 (2H, m), 4.02 (3H, s), 7.02 (1H, s), 7.81 (1H, d, J=8.8 Hz), 8.12 (1H, s), 8.15 (1H, d, J=8.8 Hz), 8.26 (1H, s), 8.44 (1H, s), 8.85 (1H, s).

### Step 4: Preparation of 8-(1-methyl-1H-pyrazol-4-yl)-2-(1-methylpiperidin-4-yl)-3H-pyrrolo[2, 3-c]isoquinoline (Compound 40)

To a solution of Compound 39 (150 mg, 0.453 mmol, TFA salt) in MeOH (4 mL) was added 37% aqueous formaldehyde (68 mg, 2.26 mmol) at 25° C. After the addition, the mixture was stirred at 25° C. for 30 minutes and then NaBH₃CN (85 mg, 1.4 mmol) was added. The resulting mixture was stirred at 25° C. for another 1 hour. The reaction mixture was concentrated and the residue was purified by prep-HPLC (0.225% FA as an additive), then lyophilized to give Compound 40 (14.54 mg, yield: 9%, FA salt) as a brown solid.

¹H NMR (400 MHz, CD₃OD) δ 2.05-2.20 (2H, m), 2.30-2.45 (2H, m), 2.89 (3H, s), 3.05-3.20 (3H, m), 3.55-3.65 (2H, m), 3.95 (3H, s), 6.83 (1H, s), 7.60 (1H, dd, J=8.4, 0.8 Hz), 7.96 (1H, d, J=8.4 Hz), 8.00 (1H, s), 8.08 (1H, s), 8.25 (1H, s), 8.56 (1H, s), 8.67 (1H, s).

### Example 25. Preparation of Compound 41 and 42

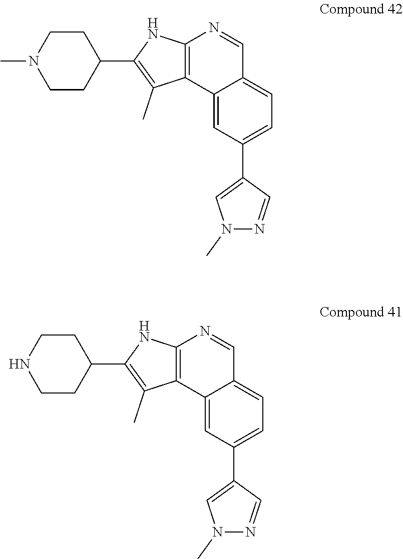 [SMILES: Cc1c(C2CCN(C)CC2)[nH]c2ncc3ccc(-c4cnn(C)c4)cc3c12 ; Cc1c(C2CCNCC2)[nH]c2ncc3ccc(-c4cnn(C)c4)cc3c12]

### Step 1: Preparation of tert-butyl 4-(1-methyl-8-(1-methyl-1H-pyrazol-4-yl)-3H-pyrrolo[2,3-c]isoquinolin-2-yl)piperidine-1-carboxylate

A mixture of Int-5 (200 mg, 0.571 mmol), Int-15 (383 mg, 1.71 mmol), Pd(dppf)Cl₂ (42 mg, 0.057 mmol), KOAc (112 mg, 1.14 mmol) and LiCl (24 mg, 0.057 mmol) in anhydrous DMF (8 mL) was degassed and purged with N₂ for 3 times. Then the mixture was stirred at 105° C. for 12 hours under N₂ atmosphere. The reaction mixture was concentrated and the residue was purified by Combi Flash (0% to 75% EtOAc in PE) to give tert-butyl 4-(1-methyl-8-(1-methyl-1H-pyrazol-4-yl)-3H-pyrrolo[2,3-c]isoquinolin-2-yl)piperidine-1-carboxylate (131 mg, yield: 51%) as a yellow solid.

¹H NMR (400 MHz, DMSO-d₆) δ 1.40-1.44 (1H, m), 1.45 (9H, s), 1.60-1.70 (3H, m), 1.80-1.90 (3H, m), 2.64 (3H, s), 3.10-3.20 (1H, m), 3.90-3.95 (3H, m), 4.10-4.20 (1H, m), 7.60-7.70 (1H, m), 7.95-8.00 (2H, m), 8.35-8.40 (2H, m), 8.72 (1H, s), 11.57 (1H, brs).

### Step 2: Preparation of 1-methyl-8-(1-methyl-1H-pyrazol-4-yl)-2-(piperidin-4-yl)-3H-pyrrolo[2, 3-c]isoquinoline (Compound 41)

A mixture of tert-butyl 4-(1-methyl-8-(1-methyl-1H-pyrazol-4-yl)-3H-pyrrolo[2,3-c]isoquinolin-2-yl)piperidine-1-carboxylate (120 mg, 0.269 mmol) in TFA (1 mL) and DCM (4 mL) was stirred at 25° C. for 2 hours. The reaction mixture was concentrated and the residue was purified by prep-HPLC (0.1% TFA as an additive), then lyophilized to give Compound 41 (20.2 mg, yield: 21%, TFA salt) as a yellow solid.

¹H NMR (400 MHz, CD₃OD) δ 2.10-2.20 (4H, m), 2.76 (3H, s), 3.25-3.30 (2H, m), 3.40-3.50 (1H, m), 3.60-3.70 (2H, m), 4.02 (3H, s), 7.78 (1H, dd, J=8.4, 2.8 Hz), 8.05 (1H, d, J=0.8 Hz), 8.15 (1H, d, J=8.8 Hz), 8.23 (1H, s), 8.56 (1H, s), 8.79 (1H, s).

### Step 3: Preparation of 1-methyl-8-(1-methyl-1H-pyrazol-4-yl)-2-(1-methylpiperidin-4-yl)-3H-pyrrolo[2,3-c]isoquinoline (Compound 42)

To a solution of Compound 41(180 mg, 0.521 mmol, TFA salt) in MeOH (5 mL) was added 37% aqueous formaldehyde (211 mg, 2.61 mmol) at 25° C. Then the mixture was stirred at 25° C. for 30 minutes, then NaBH₃CN (98 mg, 1.6 mmol) was added and the resulting mixture was stirred at 25° C. for another 1 hour. The reaction mixture was concentrated and the residue was purified by prep-HPLC (0.225% FA as an additive), then lyophilized to give Compound 42 (7.88 mg, yield: 4%, FA salt) as a yellow solid.

¹H NMR (400 MHz, CD₃OD) δ 2.00-2.35 (4H, m), 2.64 (3H, s), 2.88 (3H, s), 3.05-3.20 (2H, m), 3.35-3.40 (1H, m), 3.55-3.65 (2H, m), 3.94 (3H, s), 7.55-7.65 (1H, m), 7.94 (1H, s), 8.00 (1H, d, J=8.4 Hz), 8.10 (1H, s, HCOOH), 8.42 (1H, s), 8.47 (1H, s), 8.64 (1H s).

### Example 26. Preparation of Compound 43

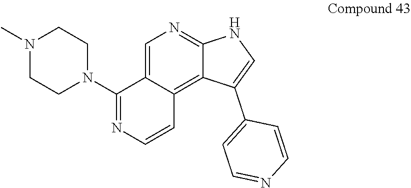 [SMILES: CN1CCN(c2nccc3c2cnc2[nH]cc(-c4ccncc4)c23)CC1]

### Step 1: Preparation of (E)-2,6-dichloro-4-(2-(dimethylamino)vinyl)nicotinonitrile

A solution of 2,6-dichloro-4-methylnicotinonitrile (30.0 g, 160 mmol) and DMF-DMA (38.2 g, 321 mmol) in DMF (300 mL) was stirred at 100° C. for 3 hours. The reaction mixture was concentrated. The residue was diluted with EtOAc (180 mL) and H₂O (600 mL) and stirred at 25° C. for 30 min, a yellow solid was precipitated. The solid was filtered and washed with EtOAc (60 mL×2) and the filtered cake was dried to give (E)-2,6-dichloro-4-(2-(dimethylamino)vinyl)nicotinonitrile (18.0 g, yield: 46%) as a gray solid.

### Step 2: Preparation of 6,8-dichloro-2,7-naphthyridin-1(2H)-one

A solution of (E)-2,6-dichloro-4-(2-(dimethylamino)vinyl)nicotinonitrile (18.0 g, 74.4 mmol) in aqueous 12N HCl (95 mL) was stirred at 45° C. for 15 hours. After cooled to room temperature, ice water (200 mL) was added and the precipitate was collected by filtration, washed with water (150 mL), EtOAc (130 mL) and dried to give 6,8-dichloro-2,7-naphthyridin-1(2H)-one (11.0 g, yield: 69%) as a yellow solid.

¹H NMR (400 MHz, DMSO-d₆) δ 6.53 (1H, d, J=6.8 Hz), 7.51 (1H, t, J=6.4 Hz), 7.77 (1H, s), 11.77 (1H, brs).

### Step 3: Preparation of 6-chloro-2,7-naphthyridin-1(2H)-one

To a solution of 6,8-dichloro-2,7-naphthyridin-1(2H)-one (11.0 g, 51.2 mmol) in i-PrOH (420 mL) was added hydrazine hydrate (24.5 g, 613 mmol, 80% purity) dropwise at 0° C., the mixture was stirred at 25° C. for 0.5 hour and stirred at 55° C. for 12 hours. The reaction mixture was cooled to room temperature, the solid was filtered and washed with MeOH (200 mL), dried to give the intermediate (10.6 g) as a yellow solid, which was suspended in MeCN (660 mL). 1N aqueous NaOH (126 mL) and H₂O (792 mL) were added and the mixture was stirred at 50° C. for 3 hours. The reaction mixture was cooled to 0° C. 9% aqueous NaClO (104 g, 126 mmol) was added dropwise to the mixture, the reaction mixture was stirred at 25° C. for 12 hours. The reaction mixture was cooled to 0° C. and acidified with 1N aqueous HCl to pH=6, then filtered. The filtrated was extracted with EtOAc (800 mL×3), the combined organic layer was dried over anhydrous Na₂SO₄, filtered and concentrated. The crude product was triturated with EtOAc (60 mL), filtered and dried to give 6-chloro-2,7-naphthyridin-1(2H)-one (7.50 g, yield: 83% for two steps) as a brown solid.

### Step 4: Preparation of 2-benzyl-6-chloro-2,7-naphthyridin-1(2H)-one

To a solution of 6-chloro-2,7-naphthyridin-1(2H)-one (5.50 g, 30.5 mmol) and Cs₂CO₃ (11.9 g, 36.5 mmol) in DMF (80 mL) was added BnCl (4.05 g, 32.0 mmol), the mixture was stirred at 25° C. for 12 hours. The reaction mixture was concentrated and the residue was diluted with H₂O (300 mL), extracted with EtOAc (300 mL×3). The combined organic layer was dried over anhydrous Na₂SO₄, filtered and concentrated. The residue was purified by flash silica gel chromatography (ISCO®; 40 g SepaFlash® Silica Flash Column, Eluent of 16˜55% Ethyl acetate/Petroleum ether gradient @65 mL/min) to give 2-benzyl-6-chloro-2,7-naphthyridin-1(2H)-one (4.40 g, yield: 53%) as a yellow solid.

### Step 5: Preparation of tert-butyl (7-benzyl-8-oxo-7,8-dihydro-2,7-naphthyridin-3-yl)carbamate

To a solution of 2-benzyl-6-chloro-2,7-naphthyridin-1(2H)-one (4.40 g, 16.3 mmol) and BocNH₂ (2.86 g, 24.4 mmol) in 1, 4-dioxane (60 mL) was added XPhos (1.55 g, 3.25 mmol), Cs₂CO₃ (10.6 g, 32.5 mmol) and Pd₂(dba)₃ (1.49 g, 1.63 mmol) under N₂ atmosphere, the mixture was stirred at 90° C. for 16 hours under N₂ atmosphere. The reaction mixture was diluted into H₂O (300 mL), extracted with EtOAc (300 mL×3). The aqueous phase was filtered and the filtered cake was dried to give tert-butyl (7-benzyl-8-oxo-7,8-dihydro-2,7-naphthyridin-3-yl)carbamate (4.00 g, yield: 70%) as a yellow solid.

### Step 6: Preparation of 6-amino-2-benzyl-2,7-naphthyridin-1 (2H)-one

To a solution of tert-butyl (7-benzyl-8-oxo-7,8-dihydro-2,7-naphthyridin-3-yl)carbamate (4.00 g, 11.4 mmol) in DCM (50 mL) was added TFA (12 mL), the mixture was stirred at 25° C. for 3 hours. The reaction mixture was concentrated and the residue was diluted with H₂O (120 mL) and DCM (120 mL), then basified with saturated aqueous Na₂CO₃ to pH =9. A light yellow solid was collected by filtration and the filtrate was extracted with DCM (60 mL×2). The combined organic layer was dried over anhydrous Na₂SO₄, filtered and concentrated to give the desired compound. The two batches were combined to give 6-amino-2-benzyl-2,7-naphthyridin-1(2H)-one (2.80 g, yield: 98%) as a yellow solid.

¹H NMR (400 MHz, DMSO-d₆) δ 5.05 (2H, s), 6.23-6.35 (2H, m), 6.57 (2H, brs), 7.22-7.38 (5H, m), 7.46 (1H, d, J=7.6 Hz), 8.82 (1H, s).

### Step 7: Preparation of 6-amino-2-benzyl-5-iodo-2,7-naphthyridin-1(2H)-one

A solution of 6-amino-2-benzyl-2,7-naphthyridin-1(2H)-one (2.80 g, 11.1 mmol) and NIS (3.01 g, 13.4 mmol) in DMF (30 mL) was stirred at 0° C. for 5 hours and the reaction mixture was stirred at 25° C. for 9 hours. The reaction mixture was concentrated and the residue was purified by flash silica gel chromatography (ISCO®; 40 g SepaFlash® Silica Flash Column, Eluent of 10˜35% Ethyl acetate/dichloromethane gradient @40 mL/min) to give 6-amino-2-benzyl-5-iodo-2,7-naphthyridin-1(2H)-one (1.40 g, yield: 33%) as a yellow solid.

### Step 8: Preparation of 7-benzyl-1-(pyridin-4-yl)-2-(trimethylsilyl)-3,7-dihydro-6H-pyrrolo[2,3-c][2, 7]naphthyridin-6-one

To a solution of 6-amino-2-benzyl-5-iodo-2,7-naphthyridin-1(2H)-one (550 mg, 1.46 mmol) and Int-16 (767 mg, 4.37 mmol) in DMF (10 mL) was added Pd(PPh₃)₂Cl₂ (102 mg, 0.146 mmol) and DABCO (327 mg, 2.92 mmol) under N₂ atmosphere, the mixture was stirred at 145° C. for 2 hours under microwave irradiation. The reaction mixture was concentrated and the residue was purified by flash silica gel chromatography (ISCO®; 20 g SepaFlash® Silica Flash Column, Eluent of 55˜90% Ethyl acetate/Petroleum ether gradient @40 mL/min) to give 7-benzyl-1-(pyridin-4-yl)-2-(trimethylsilyl)-3,7-dihydro-6H-pyrrolo[2,3-c][2,7]naphthyridin-6-one (1.00 g, yield: 81%) as a yellow solid.

¹H NMR (400 MHz, DMSO-d₆) δ 0.12 (9H, s), 5.13 (2H, s), 6.04 (1H, d, J=7.6 Hz), 7.23-7.36 (5H, m), 7.44-7.49 (2H, m), 7.68 (1H, d, J=7.6 Hz), 8.64-8.70 (2H, m), 9.13 (1H, s), 12.24 (1H, brs).

### Step 9: Preparation of I-(pyridin-4-yl)-3, 7-dihydro-6H-pyrrolo[2,3-c][2, 7]naphthyridin-6-one

A solution of 7-benzyl-1-(pyridin-4-yl)-2-(trimethylsilyl)-3,7-dihydro-6H-pyrrolo[2,3-c][2,7]naphthyridin-6-one (1.00 g, 2.36 mmol) in TfOH (10 mL) was stirred at 120° C. for 16 hours. The reaction mixture was quenched with 2N aqueous NaOH (30 mL) dropwise at 0° C., the precipitate formed was filtered and dried to give 1-(pyridin-4-yl)-3,7-dihydro-6H-pyrrolo[2,3-c][2,7]naphthyridin-6-one (800 mg, crude) as a gray solid, which was used into the next step without further purification.

¹H NMR (400 MHz, DMSO-d₆) δ 6.59 (1H, d, J=7.6 Hz), 7.39 (1H, d, J=7.2 Hz), 7.55-7.57 (2H, m), 7.77 (1H, s), 8.64 (2H, d, J=6.0 Hz), 9.10 (1H, s), 11.50 (1H, brs), 12.50 (1H, brs).

### Step 10: Preparation of 6-chloro-1-(pyridin-4-yl)-3H-pyrrolo[2,3-c][2, 7]naphthyridine

A solution of 1-(pyridin-4-yl)-3,7-dihydro-6H-pyrrolo[2,3-c][2,7]naphthyridin-6-one (650 mg, crude) in POCl₃ (15 mL) was stirred at 90° C. for 18 hours. The reaction mixture was concentrated and the residue was diluted with DCM (300 mL), basified with saturated aqueous NaHCO₃ (200 mL) to pH=8 at 0° C. and separated. The organic layer was dried over anhydrous Na₂SO₄, filtered and concentrated to give 6-chloro-1-(pyridin-4-yl)-3H-pyrrolo[2,3-c][2,7]naphthyridine (800 mg, crude) as a brown solid.

### Step 11: Preparation of 6-(4-methylpiperazin-1-yl)-1-(pyridin-4-yl)-3H-pyrrolo[2,3-c][2, 7]naphthyridine (Compound 43)

To a solution of 6-chloro-1-(pyridin-4-yl)-3H-pyrrolo[2,3-c][2,7]naphthyridine (150 mg, crude) and 1-methylpiperazine (161 mg, 1.60 mmol) in DMF (3 mL) was added DIPEA (345 mg, 2.67 mmol), the mixture was stirred at 100° C. for 16 hours. The reaction mixture was diluted with H₂O (35 mL), extracted with DCM (35 mL×2). The combined organic layer was dried over anhydrous Na₂SO₄, filtered and concentrated. The residue was purified by flash silica gel chromatography (ISCO®; 4 g SepaFlash® Silica Flash Column, Eluent of 17-22% DCM/MeOH gradient @30 mL/min), then further purified by prep-HPLC (0.04% NH₃H₂O+10 mM NH₄HCO₃ as an additive) and lyophilized to give Compound 43 (21 mg, yield: 2% for three steps) as a white solid.

¹H NMR (400 MHz, DMSO-d₆) δ 2.29 (3H, s), 2.56-2.65 (4H, m), 3.41-3.51 (4H, m), 7.39 (1H, d, J=6.0 Hz), 7.59-7.64 (2H, m), 7.78 (1H, s), 8.16 (1H, d, J=6.0 Hz), 8.62-8.68 (2H, m), 9.09 (1H, s), 12.67 (1H, brs).

### Example 27. Preparation of Compound 44

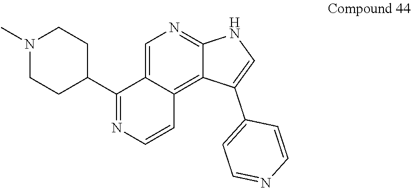 [SMILES: CN1CCC(c2nccc3c2cnc2[nH]cc(-c4ccncc4)c23)CC1]

### Step 1: Preparation of 6-(1-methyl-1,2,3,6-tetrahydropyridin-4-yl)-1-(pyridin-4-yl)-3H-pyrrolo[2,3-c][2, 7]naphthyridine

To a 6-chloro-1-(pyridin-4-yl)-3H-pyrrolo[2,3-c][2,7]naphthyridine (200 mg, 0.712 mmol) and 1-methyl-4-(4,4,5,5-tetramethyl-1,3,2-dioxaborolan-2-yl)-1,2,3,6-tetrahydropyridine (318 mg, 1.42 mmol) in 1, 4-dioxane (3 mL) and H₂O (0.6 mL) was added Pd(dppf)Cl₂ (52 mg, 0.071 mmol) and Na₂CO₃ (151 mg, 1.42 mmol). The mixture was bubbled with N₂ for 6 minutes at 25° C. and stirred at 90° C. for 16 hours under N₂ atmosphere. The reaction mixture was diluted with H₂O (35 mL), extracted with DCM (35 mL×2). The combined organic layer was dried over anhydrous Na₂SO₄, filtered and concentrated. The residue was purified by prep-HPLC (0.225% FA as an additive) to give 6-(1-methyl-1,2,3,6-tetrahydropyridin-4-yl)-1-(pyridin-4-yl)-3H-pyrrolo[2,3-c][2,7]naphthyridine (30 mg, yield: 10%, FA salt) as yellow gum.

### Step 2: Preparation of 6-(1-methylpiperidin-4-yl)-1-(pyridin-4-yl)-3H-pyrrolo[2,3-c][2, 7]naphthyridine (Compound 44)

A solution of 6-(1-methyl-1,2,3,6-tetrahydropyridin-4-yl)-1-(pyridin-4-yl)-3H-pyrrolo[2,3-c][2,7]naphthyridine (25 mg, 0.065 mmol, FA salt), PtO₂ (25 mg, 0.11 mmol) and Et₃N (33 mg, 0.32 mmol) in MeOH (30 mL) was degassed and purged with Ar for 3 times and then charged with H₂, the mixture was stirred at 25° C. for 22 hours under H₂ (50 psi) atmosphere. The reaction mixture was filtered and the filtrate was concentrated. The residue was purified by prep-HPLC (0.05% NH₃H₂O+10 mM NH₄HCO₃ as an additive) and then further purified by prep-HPLC (0.225% FA as an additive) and lyophilized to give Compound 44 (4 mg, yield: 14%, FA salt) as a yellow solid.

¹H NMR (400 MHz, DMSO-d₆) δ 1.82-1.93 (2H, m), 2.00-2.14 (2H, m), 2.27-2.38 (5H, m), 2.94-3.04 (2H, m), 3.81-3.87 (1H, m), 7.60-7.68 (2H, m), 7.75-7.83 (2H, m), 8.23 (1H, s), 8.53 (1H, d, J=6.0 Hz), 8.64-8.71 (2H, m), 9.46 (1H, s), 12.77 (1H, brs).

### Example 28. Preparation of Compound 45, 46 and 47

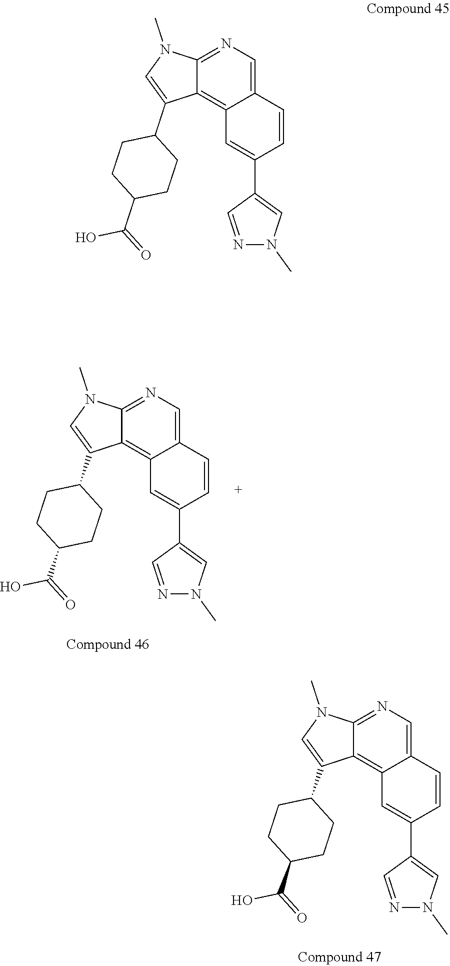 [SMILES: Cn1cc(-c2ccc3cnc4c(c(C5CCC(C(=O)O)CC5)cn4C)c3c2)cn1 ; Cn1cc(-c2ccc3cnc4c(c3c2)c([C@H]2CC[C@@H](C(=O)O)CC2)cn4C)cn1 ; Cn1cc(-c2ccc3cnc4c(c3c2)c([C@H]2CC[C@H](C(=O)O)CC2)cn4C)cn1]

### Step 1: Preparation of 4-(3-methyl-8-(1-methyl-1H-pyrazol-4-yl)-3H-pyrrolo[2,3-c]isoquinolin-1-yl)cyclohexane-1-carboxylic acid (Compound 45)

To a solution of Compound 24 (240 mg, 0.640 mmol) in DMF (5 mL) was added NaH (51 mg, 1.3 mmol, 60% dispersion in mineral oil). The mixture was stirred at 0° C. for 0.5 hour, then MeI (136 mg, 0.960 mmol) was added to the mixture and stirred at 0° C. for 0.5 hour. The reaction mixture was quenched by addition H₂O (1 mL) and then NaOH (51 mg, 1.28 mmol) was added and stirred at 25° C. for 12 hours. The reaction mixture was concentrated and the residue was acidified with 1N aqueous HCl to pH=5 and filtered. The solid was washed with water (5 mL×2) and dried to give Compound 45 (220 mg, yield: 88%) as a yellow solid.

### Step 2: SFC Purification

Compound 45 (100 mg, 0.260 mmol) was purified by SFC (condition: column DAICEL CHIRALPAK AD (250 mm*30 mm, 10 um); mobile phase: [0.1% NH₃H₂O IPA]; B %: 50%-50%), then lyophilized to give Compound 46 (26.8 mg, yield: 26%) as a yellow solid and Compound 47 (40.34 mg, yield: 40% r) as an off-white solid.

Compound 46: ¹H NMR (400 MHz, DMSO-d₆) δ 1.56-1.71 (2H, m), 1.83-1.95 (2H, m), 2.00-2.12 (2H, m), 2.15-2.25 (2H, m), 2.68-2.77 (1H, m), 3.4-3.48 (1H, m), 3.89 (3H, s), 3.94 (3H, s), 7.28 (1H, s), 7.73 (1H, dd, J=8.4, 1.2 Hz), 8.02 (1H, s), 8.13 (1H, d, J=8.8 Hz), 8.27 (1H, s), 8.31 (1H, s), 8.84 (1H, s).

Compound 47: ¹H NMR (400 MHz, DMSO-d₆) δ 1.43-1.55 (2H, m), 1.70-1.80 (2H, m), 2.00-2.15 (2H, m), 2.21-2.37 (3H, m), 3.25-3.30 (1H, m), 3.90 (3H, s), 3.94 (3H, s), 7.29 (1H, s), 7.72 (1H, dd, J=8.8, 1.6 Hz), 8.01 (1H, s), 8.14 (1H, d, J=8.4 Hz), 8.26 (1H, s), 8.32 (1H, s), 8.85 (1H, s).

### Example 29. Preparation of Compound 51

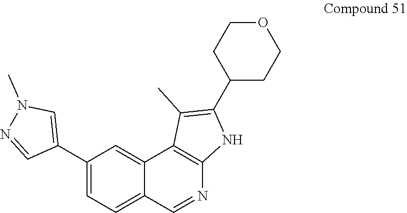 [SMILES: Cc1c(C2CCOCC2)[nH]c2ncc3ccc(-c4cnn(C)c4)cc3c12]

### Step 1: Preparation of 4-(prop-1-yn-1-yl)tetrahydro-2H-pyran

To a solution of 4-ethynyltetrahydro-2H-pyran (500 mg, 4.54 mmol) in THF (15 mL) was added n-BuLi (2.4 mL, 6.00 mmol, 2.5 M in hexane) dropwise at −65° C. After the addition, the mixture was warmed up to 25° C., and then MeI (966 mg, 6.81 mmol) was added dropwise at 25° C. The resulting mixture was stirred at 25° C. for 12 hours. The reaction mixture was quenched by addition H₂O (25 mL) and then extracted with EtOAc (25 mL×2). The combined organic layers were dried over anhydrous Na₂SO₄, filtered and concentrated. The residue was purified by Combi Flash (0% to 6% EtOAc in PE) to give 4-(prop-1-yn-1-yl)tetrahydro-2H-pyran (230 mg, yield: 41%) as colorless oil.

### Step 2: Preparation of 1-methyl-8-(1-methyl-1H-pyrazol-4-yl)-2-(tetrahydro-2H-pyran-4-yl)-3H-pyrrolo[2, 3-c]isoquinoline (Compound 51)

A mixture of Int-5 (100 mg, 0.286 mmol), 4-(prop-1-yn-1-yl)tetrahydro-2H-pyran (106 mg, 0.857 mmol), Pd(dppf)Cl₂ (21 mg, 0.286 mmol), LiCl (12 mg, 0.286 mmol) and KOAc (56 mg, 0.571 mmol) in DMF (4 mL) was degassed and purged with N₂ for 3 times, and then the mixture was stirred at 100° C. for 12 hours under N₂ atmosphere. The reaction mixture was concentrated and the residue was purified by prep-HPLC (0.225% FA as an additive) then lyophilized to give Compound 51 (13.5 mg, yield: 13%) as a yellow solid.

¹H NMR (400 MHz, DMSO-d₆) δ 1.60-1.68 (2H, m), 1.95-2.09 (2H, m), 2.65 (3H, s), 3.23-3.26 (1H, m), 3.47-3.57 (2H, m), 3.93 (3H, s), 3.95-4.03 (2H, m), 7.69 (1H, dd, J=8.8, 1.6 Hz), 8.05 (1H, s), 8.07 (1H, d, J=8.4 Hz), 8.36-8.43 (2H, m), 8.73 (1H, s), 11.61 (1H, brs).

### Example 30. Preparation of Compound 52

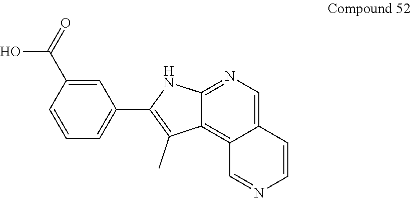 [SMILES: Cc1c(-c2cccc(C(=O)O)c2)[nH]c2ncc3ccncc3c12]

### Step 1: Preparation of ethyl 3-(9-methyl-7H-pyrrolo[2,3-c][2,6]naphthyridin-8-yl)benzoate

A mixture of Int-13 (100 mg, 0.369 mmol), Int-18 (139 mg, 0.738 mmol), Pd(dppf)Cl₂ (27 mg, 0.037 mmol), LiCl (16 mg, 0.37 mmol) and KOAc (72 mg, 0.74 mmol) in DMF (5 mL) was degassed and purged with N₂ for 3 times, and then the mixture was stirred at 105° C. for 16 hours under N₂ atmosphere. The reaction mixture was diluted with H₂O (20 mL) and extracted with EtOAc (20 mL×3). The combined organic layers were washed with brine (30 mL), dried over anhydrous Na₂SO₄, filtered and concentrated. The residue was purified by silica gel column (0% to 60% EtOAc in PE) to give ethyl 3-(9-methyl-7H-pyrrolo[2,3-c][2,6]naphthyridin-8-yl)benzoate (55 mg, yield: 35%) as a yellow solid.

### Step 2: Preparation of 3-(9-methyl-7H-pyrrolo[2, 3-c][2,6]naphthyridin-8-yl)benzoic acid (Compound 52)

To a solution of ethyl 3-(9-methyl-7H-pyrrolo[2,3-c][2,6]naphthyridin-8-yl)benzoate (55 mg, 0.17 mmol) in THF (2 mL), MeOH (2 mL) and H₂O (1 mL) was added LiOH·H₂O (70 mg, 1.7 mmol). The mixture was stirred at 25° C. for 1 hour. The reaction mixture was concentrated and the residue was acidified with 1N aqueous HCl to pH=5 and filtered. The solid was washed with water (5 mL×2) and dried, then further purified by prep-HPLC (0.225% FA as an additive) and lyophilized to afford Compound 52 (2.08 mg, yield: 4%) as a yellow solid.

¹H NMR (400 MHz, DMSO-d₆) δ 2.80 (3H, s), 7.68 (1H, t, J=8.0 Hz), 7.92 (1H, d, J=7.6 Hz), 8.00 (1H, d, J=7.6 Hz), 8.06-8.10 (1H, m), 8.25 (1H, s), 8.65 (1H, d, J=5.2 Hz), 9.03 (1H, s), 9.90 (1H, s), 12.63 (1H, brs).

The following compound was synthesized analogously to Compound 52

| Comp. | | | |
|---|---|---|---|
| No | Structure | Name | ¹H NMR (400 MHz) |
| 95 | 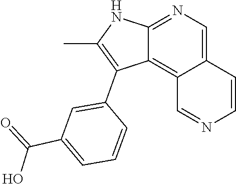 [SMILES: Cc1[nH]c2ncc3ccncc3c2c1-c1cccc(C(=O)O)c1] | 3-(8-methyl-7H- pyrrolo[2,3- c][2,6]naphthyridin- 9-yl)benzoic acid | DMSO-d₆; δ 2.42 (3H, s), 7.67- 7.73 (1H, m), 7.81 (1H, d, J = 7.6 Hz), 8.00-8.10 (3H, m), 8.51 (1H, d, J = 5.6 Hz), 8.97 (1H, s), 9.14 (1H, s), 12.51 (1H, brs). |

### Example 31. Preparation of Compound 53

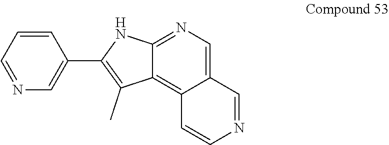 [SMILES: Cc1c(-c2cccnc2)[nH]c2ncc3cnccc3c12]

A mixture of Int-19 (120 mg, 0.443 mmol), 3-(prop-1-yn-1-yl)pyridine (156 mg, 1.33 mmol), DABCO (99 mg, 0.89 mmol) and Pd(PPh₃)₂Cl₂ (31 mg, 0.044 mmol) in DMF (2 mL) was stirred at 145° C. under microwave irradiation for 2 hours under N₂ atmosphere. The reaction mixture was concentrated and the residue was purified by silica gel column (PE/EtOAc =1/1), followed by prep-TLC (DCM/MeOH=10/1), then further purified by prep-HPLC (0.225% FA as an additive) and lyophilized to afford Compound 53 (1 mg, yield: 1%) as a yellow solid.

¹H NMR (400 MHz, DMSO-d₆) δ 2.74 (3H, s), 7.58 (1H, dd, J=7.2, 4.4 Hz), 8.07-8.10 (1H, m), 8.28 (1H, d, J=6.0 Hz), 8.62 (1H, dd, J=4.8, 1.6 Hz), 8.72 (1H, d, J=6.0 Hz), 8.90 (1H, d, J=2.0 Hz), 9.13 (1H, s), 9.48 (1H, s), 12.66 (1H, brs).

### Example 32. Preparation of Compound 54

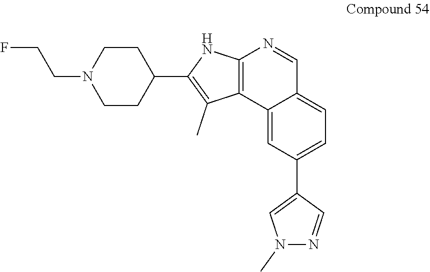 [SMILES: Cc1c(C2CCN(CCF)CC2)[nH]c2ncc3ccc(-c4cnn(C)c4)cc3c12]

### Step 1: Preparation of tert-butyl 4-(1-methyl-8-(1-methyl-1H-pyrazol-4-yl)-3H-pyrrolo[2,3-c]isoquinolin-2-yl)piperidine-1-carboxylate

A mixture of Int-5 (1.00 g, 2.86 mmol), Int-20 (1.91 g, 8.57 mmol), Pd(dppf)Cl₂ (209 mg, 0.286 mmol), KOAc (561 mg, 5.71 mmol) and LiCl (121 mg, 2.86 mmol) in DMF (20 mL) was degassed and purged with N₂ for 3 times, and then the mixture was stirred at 105° C. for 12 hours under N₂ atmosphere. The reaction mixture was concentrated and the residue was diluted with H₂O (50 mL), then extracted with EtOAc (50 mL×4). The combined organic layer was washed with brine (100 mL), dried over anhydrous Na₂SO₄, filtered and concentrated. The residue was purified by flash silica gel chromatography (EtOAc as eluent) to afford tert-butyl 4-(1-methyl-8-(1-methyl-TH-pyrazol-4-yl)-3H-pyrrolo[2,3-c]isoquinolin-2-yl)piperidine-1-carboxylate (650 mg, yield: 51%) as a yellow solid.

¹H NMR (400 MHz, DMSO-d₆) δ 1.40-1.45 (10H, m), 1.65-1.75 (2H, m), 1.77-1.87 (2H, m), 2.63 (3H, s), 3.09-3.21 (2H, m), 3.92 (3H, s), 4.07-4.11 (1H, m), 7.68 (1H, dd, J=8.4, 1.6 Hz), 8.03-8.09 (2H, m), 8.34-8.41 (2H, m), 8.71 (1H, s), 11.56 (1H, brs).

### Step 2: Preparation of 1-methyl-8-(1-methyl-1H-pyrazol-4-yl)-2-(piperidin-4-yl)-3H-pyrrolo[2,3-c]isoquinoline

A mixture of tert-butyl 4-(1-methyl-8-(1-methyl-TH-pyrazol-4-yl)-3H-pyrrolo[2,3-c]isoquinolin-2-yl)piperidine-1-carboxylate (650 mg, 1.46 mmol) in DCM (12 mL) and TFA (12 mL) was stirred at 25° C. for 1 hour. The reaction mixture was concentrated and the residue was diluted with H₂O (30 mL), then basified with saturated aqueous NaHCO₃ to pH=8, filtered and dried to give 1-methyl-8-(1-methyl-TH-pyrazol-4-yl)-2-(piperidin-4-yl)-3H-pyrrolo[2,3-c]isoquinoline (290 mg, yield: 58%) as a gray solid.

### Step 3: Preparation of 2-(1-(2-fluoroethyl)piperidin-4-yl)-1-methyl-8-(1-methyl-1H-pyrazol-4-yl)-3H-pyrrolo[2, 3-c]isoquinoline (Compound 54)

A mixture of 1-methyl-8-(1-methyl-1H-pyrazol-4-yl)-2-(piperidin-4-yl)-3H-pyrrolo[2,3-c]isoquinoline (75 mg, 0.22 mmol), CH₂FCH₂CH₂CH₂Br (138 mg, 1.09 mmol) and K₂CO₃ (90 mg, 0.65 mmol) in DMF (5 mL) was stirred at 80° C. for 6 hours. The reaction mixture was filtered and the filtrate was concentrated. The residue was purified by prep-HPLC (0.225% FA as an additive), then lyophilized to afford Compound 54 (15.7 mg, yield: 16%, FA salt) as a yellow solid.

¹H NMR (400 MHz, DMSO-d₆) δ 1.65-1.75 (2H, m), 1.90-2.08 (2H, m), 2.16-2.22 (2H, m), 2.62 (3H, s), 2.64-2.66 (1H, m), 2.70-2.74 (1H, m), 2.89-2.99 (1H, m), 3.00-3.10 (2H, m), 3.92 (3H, s), 4.50-4.54 (1H, m), 4.62-4.66 (1H, m), 7.67 (1H, dd, J=8.4, 1.6 Hz), 8.04 (1H, s), 8.06 (1H, d, J=8.4 Hz), 8.36 (1H, s), 8.39 (1H, s), 8.71 (1H, s), 11.55 (1H, brs).

The following compounds were synthesized analogously to Compound 54

| Comp. | | | |
|---|---|---|---|
| No | Structure | Name | ¹H NMR (400 MHz) |
| 55 | 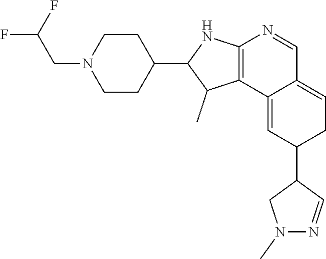 [SMILES: CC1c2c(ncc3c2=CC(C2C=NN(C)C2)CC=3)NC1C1CCN(CC(F)F)CC1] | 2-(1-(2,2- difluoroethyl)piperidin- 4-yl)-1-methyl-8- (1-methyl-1H- pyrazol-4-yl)-3H- pyrrolo[2,3- c]isoquinoline | DMSO-d₆; δ 1.65-1.75 (2H, m), 2.00-2.04 (2H, m), 2.21- 2.45 (2H, m), 2.62 (3H, s), 2.69-2.88 (2H, m), 2.96-2.98 (1H, m), 3.05-3.08 (2H, m), 3.92 (3H, s), 6.01-6.39 (1H, m), 7.68 (1H, dd, J = 8.4, 1.6 Hz), 8.04 (1H, s), 8.06 (1H, d, J = 8.4 Hz), 8.36 (1H, s), 8.39 (1H, s), 8.71 (1H, s), 11.57 (1H, brs). |
| 56 | 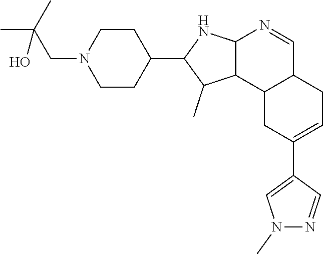 [SMILES: CC1C(C2CCN(CC(C)(C)O)CC2)NC2N=CC3CC=C(c4cnn(C)c4)CC3C21] | 2-methyl-1-(4-(1- methyl-8-(1-methyl- 1H-pyrazol-4-yl)-3H- pyrrolo[2,3- c]isoquinolin-2- yl)piperidin-1- yl)propan-2-ol | DMSO-d₆; δ 1.13 (6H, s), 1.60-1.68 (2H, m), 1.91-2.10 (2H, m), 2.24-2.29 (3H, m), 2.29-2.36 (1H, m), 2.61 (3H, s), 2.84-2.93 (1H, m), 3.07- 3.12 (2H, m), 3.92 (3H, s), 4.07 (1H, s), 7.67 (1H, dd, J = 8.4, 1.6 Hz), 8.04 (1H, s), 8.06 (1H, d, J = 8.4 Hz), 8.36 (1H, s), 8.39 (1H, s), 8.70 (1H, s), 11.53 (1H, brs). |

### Example 33. Preparation of Compound 57

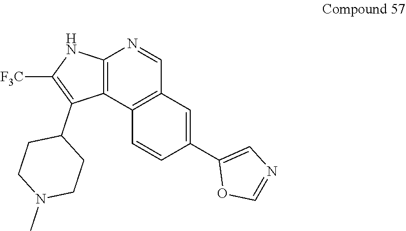 [SMILES: CN1CCC(c2c(C(F)(F)F)[nH]c3ncc4cc(-c5cnco5)ccc4c23)CC1]

### Step 1: Preparation of methyl 3-chloroisoquinoline-7-carboxylate

A mixture of 7-bromo-3-chloroisoquinoline (1.75 g, 7.22 mmol), Pd(dppf)Cl₂·CH₂Cl₂ (1.18 g, 1.44 mmol) and Et₃N (3.65 g, 36.1 mmol) in MeOH (50 mL) and DMF (10 mL) was degassed and purged with CO for three times, the mixture was stirred at 70° C. under CO (50 psi) atmosphere for 20 hours. The reaction mixture turned into yellow suspension. After cooled to room temperature, the reaction mixture was concentrated. The residue was purified by flash silica gel chromatography (ISCO®R; 40 g SepaFlash® Silica Flash Column, Eluent of 7˜30% Ethyl acetate/Petroleum ether gradient @70 mL/min) to give methyl 3-chloroisoquinoline-7-carboxylate (3.00 g, yield: 94% o) as a white solid.

### Step 2: Preparation of methyl 3-((tert-butoxycarbonyl)amino)isoquinoline-7-carboxylate

To a solution of methyl 3-chloroisoquinoline-7-carboxylate (1.10 g, 4.96 mmol) and BocNH₂ (1.16 g, 9.93 mmol) in 1, 4-dioxane (30 mL) was added Pd₂(dba)₃ (454 mg, 0.496 mmol), Xantphos (574 mg, 0.992 mmol) and Cs₂CO₃ (4.85 g, 14.9 mmol) under N₂ atmosphere, the mixture was stirred at 110° C. for 16 hours under N₂ atmosphere. The reaction mixture turned into yellow suspension. The reaction mixture was concentrated and the residue was diluted with H₂O (40 mL) and EtOAc (40 mL). The precipitated was filtered and dried to give methyl 3-((tert-butoxycarbonyl)amino)isoquinoline-7-carboxylate (1.20 g, yield: 80%) as gray solid.

### Step 3: Preparation of methyl 3-aminoisoquinoline-7-carboxylate

To a solution of methyl 3-((tert-butoxycarbonyl)amino)isoquinoline-7-carboxylate (1.20 g, 3.97 mmol) in DCM (15 mL) was added TFA (15 mL) at 0° C., the mixture was stirred at 25° C. for 12 hours. The reaction mixture turned into brown solution. The reaction mixture was concentrated and the residue was diluted with H₂O (50 mL), basified with saturated aqueous NaHCO₃ to pH=8, extracted with DCM (50 mL×5). The combined organic layer was dried over anhydrous Na₂SO₄, filtered and concentrated. The residue was purified by flash silica gel chromatography (ISCO®; 4 g SepaFlash® Silica Flash Column, Eluent of 45˜80% Ethyl acetate/Petroleum ether gradient @40 mL/min) to give methyl 3-aminoisoquinoline-7-carboxylate (600 mg, yield: 73%) as a yellow solid.

¹H NMR (400 MHz, DMSO-d₆) δ 3.86 (3H, s), 6.40 (2H, brs), 6.62 (1H, s), 7.55 (1H, d, J=8.8 Hz), 7.86 (1H, dd, J=8.8, 1.6 Hz), 8.47-8.51 (1H, m), 8.99 (1H, s).

### Step 4: Preparation of methyl 3-amino-4-iodoisoquinoline-7-carboxylate

To a solution of methyl 3-aminoisoquinoline-7-carboxylate (600 mg, 2.97 mmol) in DMF (8 mL) was added NIS (734 mg, 3.26 mmol) at 0° C., the mixture was stirred at 25° C. for 3 hours to give brown solution. The reaction mixture was added into H₂O (30 mL) dropwise. The precipitated was filtered and dried to give methyl 3-amino-4-iodoisoquinoline-7-carboxylate (980 mg, crude) as a brown solid.

### Step 5: Preparation of methyl 1-(1-(tert-butoxycarbonyl)piperidin-4-yl)-2-(trifluoromethyl)-3H-pyrrolo[2,3-c]isoquinoline-7-carboxylate

To a solution of methyl 3-amino-4-iodoisoquinoline-7-carboxylate (490 mg, 1.49 mmol) and Int-21 (497 mg, 1.79 mmol) in DMF (8 mL) was added Pd(PPh₃)₄ (345 mg, 0.299 mmol) and Et₃N (453 mg, 4.48 mmol) under N₂ atmosphere, the mixture was stirred at 130° C. for 16 hours under N₂ atmosphere to give black suspension. After cooled to room temperature, the reaction mixture was concentrated and the residue was purified by flash silica gel chromatography (ISCO®; 20 g SepaFlash® Silica Flash Column, Eluent of ˜30% Ethyl acetate/Petroleum ether gradient @40 mL/min) to give methyl 1-(1-(tert-butoxycarbonyl)piperidin-4-yl)-2-(trifluoromethyl)-3H-pyrrolo[2,3-c]isoquinoline-7-carboxylate (210 mg, yield: 29%) as a yellow solid.

### Step 6: Preparation of tert-butyl 4-(7-(hydroxymethyl)-2-(trifluoromethyl)-3H-pyrrolo[2,3-c]isoquinolin-1-yl)piperidine-1-carboxylate

To a solution of methyl 1-(1-(tert-butoxycarbonyl)piperidin-4-yl)-2-(trifluoromethyl)-3H-pyrrolo[2,3-c]isoquinoline-7-carboxylate (370 mg, 0.775 mmol) in THF (10 mL) was added DIBAL-H (2.3 mL, 2.32 mmol, 1M in toluene) dropwise at 0° C. under N₂ atmosphere, the mixture was stirred at 0° C. for 3 hours under N₂ atmosphere to give brown solution. The reaction mixture was quenched with MeOH (2 mL) and concentrated. The residue was diluted with H₂O (60 mL), extracted with EtOAc (60 mL×2). The combined organic layer was dried over anhydrous Na₂SO₄, filtered and concentrated. The residue was purified by flash silica gel chromatography (ISCO®; 12 g SepaFlash® Silica Flash Column, Eluent of ˜60% Ethyl acetate/Petroleum ether gradient @40 mL/min) to give tert-butyl 4-(7-(hydroxymethyl)-2-(trifluoromethyl)-3H-pyrrolo[2,3-c]isoquinolin-1-yl)piperidine-1-carboxylate (220 mg, yield: 59%) as a yellow solid.

### Step 7: Preparation of tert-butyl 4-(7-formyl-2-(trifluoromethyl)-3H-pyrrolo[2,3-c]isoquinolin-1-yl)piperidine-1-carboxylate

To a solution of tert-butyl 4-(7-(hydroxymethyl)-2-(trifluoromethyl)-3H-pyrrolo[2,3-c]isoquinolin-1-yl)piperidine-1-carboxylate (220 mg, 0.489 mmol) in anhydrous DCM (15 mL) was added MnO₂ (340 mg, 3.92 mmol), the mixture was stirred at 45° C. for 16 hours. The reaction mixture was filtered through a pad of celite and the solid was washed with DCM (10 mL×3). The combined organic layer was concentrated and the residue was dissolved in DCM (15 mL), then MnO₂ (340 mg, 3.92 mmol) was added and the mixture was stirred at 45° C. for another 20 hours to give black suspension. The reaction mixture was filtered through a pad of celite and the solid was washed with DCM/MeOH (10 mL ×3, 10/1). The filtrate was concentrated to give tert-butyl 4-(7-formyl-2-(trifluoromethyl)-3H-pyrrolo[2,3-c]isoquinolin-1-yl)piperidine-1-carboxylate (155 mg, crude) as yellow solid.

### Step 8: Preparation of tert-butyl 4-(7-(oxazol-5-yl)-2-(trifluoromethyl)-3H-pyrrolo[2,3-c]isoquinolin-1-yl)piperidine-1-carboxylate

A mixture of tert-butyl 4-(7-formyl-2-(trifluoromethyl)-3H-pyrrolo[2,3-c]isoquinolin-1-yl)piperidine-1-carboxylate (155 mg, crude), K₂CO₃ (62 mg, 0.45 mmol) and TosMIC (81 mg, 0.42 mmol) in MeOH (8 mL) was stirred at 80° C. for 2 hours under N₂ atmosphere to give yellow mixture. The reaction mixture was concentrated and then diluted with H₂O (40 mL), extracted with EtOAc (40 mL×2). The combined organic layer was dried over anhydrous Na₂SO₄, filtered and concentrated. The residue was purified by flash silica gel chromatography (ISCO®; 4 g SepaFlash® Silica Flash Column, Eluent of ˜40% Ethyl acetate/Petroleum ether gradient @30 mL/min) to give tert-butyl 4-(7-(oxazol-5-yl)-2-(trifluoromethyl)-3H-pyrrolo[2,3-c]isoquinolin-1-yl)piperidine-1-carboxylate (90 mg, yield: 38% for two steps) as a yellow solid.

¹H NMR (400 MHz, DMSO-d₆) δ 1.48 (9H, s), 1.68-2.19 (4H, m), 2.92-3.12 (2H, m), 3.29-3.31 (1H, m), 4.11-4.23 (2H, m), 7.84 (1H, s), 8.10-8.22 (1H, m), 8.42-8.53 (1H, m), 8.56 (1H, s), 8.60 (1H, d, J=1.6 Hz), 9.19 (1H, s), 13.19 (1H, brs).

### Step 9: Preparation of 5-(1-(piperidin-4-yl)-2-(trifluoromethyl)-3H-pyrrolo[2,3-c]isoquinolin-7-yl)oxazole

To a solution of tert-butyl 4-(7-(oxazol-5-yl)-2-(trifluoromethyl)-3H-pyrrolo[2,3-c]isoquinolin-1-yl)piperidine-1-carboxylate (90 mg, 0.19 mmol) in DCM (3 mL) was added TFA (3 mL) at 0° C., the mixture was stirred at 20° C. for 3 hours to give light yellow solution. The reaction mixture was concentrated to give 5-(1-(piperidin-4-yl)-2-(trifluoromethyl)-3H-pyrrolo[2,3-c]isoquinolin-7-yl)oxazole (100 mg, crude, TFA salt) as a brown solid.

### Step 10: Preparation of 5-(1-(1-methylpiperidin-4-yl)-2-(trifluoromethyl)-3H-pyrrolo[2,3-c]isoquinolin-7-yl)oxazole (Compound 57)

5-(1-(Piperidin-4-yl)-2-(trifluoromethyl)-3H-pyrrolo[2,3-c]isoquinolin-7-yl)oxazole (100 mg, crude, TFA salt) in MeOH (8 mL) was neutralized with DIPEA, then aqueous HCHO (130 mg, 1.60 mmol, 37% purity) and HOAc (36 mg, 0.60 mmol) were added and the mixture was stirred at 20° C. for 1 hour. NaBH₃CN (38 mg, 0.60 mmol) was added to the reaction mixture and stirred at 20° C. for another 1 hour to give brown solution. The reaction mixture was concentrated and the residue was purified by prep-HPLC (0.05% NH₃H₂O+10 mM NH₄HCO₃ as an additive), then lyophilized to give Compound 57 (25 mg, yield: 32% for two steps) as an off-white solid.

¹H NMR (400 MHz, DMSO-d₆) δ 1.66-1.89 (2H, m), 2.03-2.16 (2H, m), 2.20-2.32 (4H, m), 2.52-2.58 (2H, m), 2.93-3.04 (2H, m), 7.86 (1H, s), 8.30 (1H, dd, J=8.8, 1.6 Hz), 8.48-8.71 (3H, m), 9.17 (1H, s).

### Example 34. Preparation of Compound 58

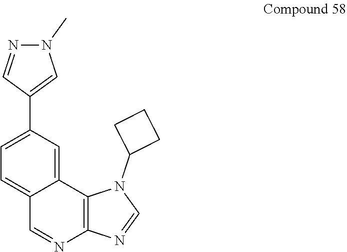 [SMILES: Cn1cc(-c2ccc3cnc4ncn(C5CCC5)c4c3c2)cn1]

### Step 1: Preparation of 2-bromo-4-(1-methyl-1H-pyrazol-4-yl)benzaldehyde

A mixture of 2-bromo-4-iodobenzaldehyde (1.00 g, 3.22 mmol), 1-methyl-4-(4,4,5,5-tetramethyl-1,3,2-dioxaborolan-2-yl)-1H-pyrazole (669 mg, 3.22 mmol), Pd(dppf)Cl₂ (2.35 g, 3.22 mmol) and Na₂CO₃ (1.02 g, 9.65 mmol) in 1, 4-dioxane (10 mL) and H₂O (1 mL) was degassed and purged with N₂ for 3 times, and then the mixture was stirred at 90° C. for 1 hour under N₂ atmosphere. The reaction mixture was concentrated and the residue was purified by flash silica gel chromatography (PE/EtOAc=3/1) to afford 2-bromo-4-(1-methyl-1H-pyrazol-4-yl)benzaldehyde (440 mg, yield: 52%) as a yellow solid.

¹H NMR (400 MHz, DMSO-d₆) δ 3.88 (3H, s), 7.74-7.79 (1H, m), 7.80-7.84 (1H, m), 8.02 (1H, s), 8.09 (1H, s), 8.41 (1H, s), 10.15 (1H, s).

### Step 2: Preparation of 4-(1-methyl-1H-pyrazol-4-yl)-2-(4,4,5,5-tetramethyl-1, 3,2-dioxaborolan-2-yl)benzaldehyde

A mixture of 2-bromo-4-(1-methyl-1H-pyrazol-4-yl)benzaldehyde (440 mg, 1.66 mmol), Bis-Pin (506 mg, 1.99 mmol), Pd(dppf)Cl₂ (121 mg, 0.166 mmol) and KOAc (489 mg, 4.98 mmol) in 1, 4-dioxane (5 mL) was degassed and purged with N₂ for 3 times, and then the mixture was stirred at 90° C. for 14 hours under N₂ atmosphere. The reaction mixture was concentrated and the residue was purified by flash silica gel chromatography (DCM/MeOH=10/1) to afford 4-(1-methyl-1H-pyrazol-4-yl)-2-(4,4,5,5-tetramethyl-1,3,2-dioxaborolan-2-yl)benzaldehyde (270 mg, yield: 33%) as yellow oil.

### Step 3: Preparation of 1-cyclobutyl-4-nitro-1H-imidazole

To a solution of 4-nitro-1H-imidazole (630 mg, 5.57 mmol) in anhydrous DMF (10 mL) was added NaH (245 mg, 6.13 mmol, 60% dispersion in mineral oil) at 20° C., the reaction mixture turned into yellow solution from white suspension. Then bromocyclobutane (1.50 g, 11.1 mmol) was added to the mixture and the mixture was stirred at 80° C. for 12 hours. The reaction mixture was quenched with saturated aqueous NH₄Cl (50 mL) and extracted with EtOAc (25 mL×3). The combined organic layer was washed with brine (25 mL), dried over anhydrous Na₂SO₄ and concentrated. The residue was purified by flash silica gel chromatography (EtOAc as eluent) to afford 1-cyclobutyl-4-nitro-1H-imidazole (360 mg, yield: 39%) as a yellow solid.

¹H NMR (400 MHz, CDCl₃) δ 1.91-2.06 (2H, m), 2.34-2.47 (2H, m), 2.56-2.67 (2H, m), 4.61-4.74 (1H, m), 7.48 (1H, d, J=1.2 Hz), 7.84 (1H, d, J=1.6 Hz).

### Step 4: Preparation of 2-(1-cyclobutyl-4-nitro-1H-imidazol-5-yl)-4-(1-methyl-1H-pyrazol-4-yl)benzaldehyde

A mixture of 1-cyclobutyl-4-nitro-1H-imidazole (220 mg, 1.32 mmol), 4-(1-methyl-1H-pyrazol-4-yl)-2-(4,4,5,5-tetramethyl-1,3,2-dioxaborolan-2-yl)benzaldehyde (384 mg, 1.45 mmol), Pd(PPh₃)₂Cl₂ (46 mg, 0.066 mmol), CuI (301 mg, 1.58 mmol), PivOH (40 mg, 0.40 mmol) and K₂CO₃ (236 mg, 1.71 mmol) in DMA (4 mL) was degassed and purged with N₂ for 3 times, and then the mixture was stirred at 130° C. for 14 hours under N₂ atmosphere. The reaction mixture was filtered and the filtrate was concentrated. The residue was purified by flash silica gel chromatography (DCM/MeOH=10/1) to afford 2-(1-cyclobutyl-4-nitro-1H-imidazol-5-yl)-4-(1-methyl-1H-pyrazol-4-yl)benzaldehyde (220 mg, yield: 48%) as a yellow solid.

### Step 5: Preparation of 1-cyclobutyl-8-(1-methyl-1H-pyrazol-4-yl)-1H-imidazo[4,5-c]isoquinoline (Compound 58)

A mixture of 2-(1-cyclobutyl-4-nitro-1H-imidazol-5-yl)-4-(1-methyl-1H-pyrazol-4-yl)benzaldehyde (220 mg, 0.626 mmol), Fe powder (140 mg, 2.50 mmol), and NH₄Cl (134 mg, 2.50 mmol) in EtOH (5 mL) and H₂O (5 mL) was stirred at 90° C. for 8 hours. The reaction mixture was poured into EtOH (20 mL) and filtered. The filtrate was concentrated and the residue was diluted with H₂O (20 mL), then extracted with EtOAc (20 mL×3). The combined organic layer was washed with brine (30 mL), dried over anhydrous Na₂SO₄, filtered and concentrated. The residue was purified by prep-HPLC (0.225% FA as an additive), then lyophilized to afford Compound 58 (42 mg, yield: 22%) as a yellow solid.

¹H NMR (400 MHz, CDCl₃) δ 2.08-2.21 (2H, m), 2.56-2.68 (2H, m), 2.78-2.88 (2H, m), 4.03 (3H, s), 5.25-5.36 (1H, m), 7.67 (1H, dd, J=8.4, 1.6 Hz), 7.79 (1H, s), 7.92 (1H, s), 8.09 (1H, d, J=8.4 Hz), 8.19 (1H, s), 8.25 (1H, s), 9.06 (1H, s).

### Example 35. Preparation of Compound 59

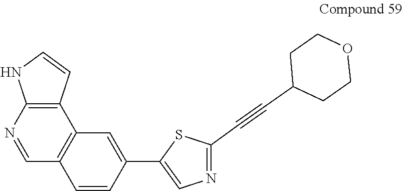 [SMILES: C(#CC1CCOCC1)c1ncc(-c2ccc3cnc4[nH]ccc4c3c2)s1]

To a solution of Int-23 (300 mg, crude) and Int-22 (416 mg, 1.53 mmol) in 1, 4-dioxane (6 mL) and H₂O (1.2 mL) was added Pd(dtbpf)Cl₂ (100 mg, 0.153 mmol) and Na₂CO₃ (270 mg, 2.55 mmol), the mixture was stirred at 100° C. for 16 hours under N₂ atmosphere to give brown suspension. The reaction mixture was concentrated and the residue was purified by flash silica gel chromatography (ISCO®; 12 g SepaFlash®Silica Flash Column, Eluent of ˜68% Ethyl acetate/Petroleum ether gradient @30 mL/min) to give Compound 59 (70 mg, yield: 42% for two steps) as a yellow solid.

¹H NMR (400 MHz, DMSO-d₆) δ 1.59-1.75 (2H, m), 1.84-1.95 (2H, m), 2.99-3.13 (1H, m), 3.43-3.53 (2H, m), 3.77-3.88 (2H, m), 7.14-7.19 (1H, m), 7.51 (1H, t, J=2.8 Hz), 7.81 (1H, dd, J=8.4, 1.6 Hz), 8.20 (1H, d, J=8.4 Hz), 8.53-8.62 (2H, m), 8.90 (1H, s), 12.07 (1H, brs).

### Example 36. Preparation of Compound 60

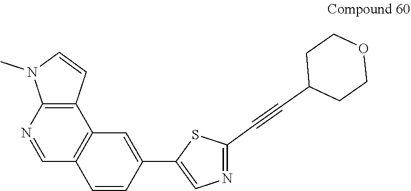 [SMILES: Cn1ccc2c3cc(-c4cnc(C#CC5CCOCC5)s4)ccc3cnc21]

To a solution of Compound 59 (50 mg, 0.14 mmol) in DMF (2 mL) was added NaH (28 mg, 0.70 mmol, 60% dispersion in mineral oil) at 0° C. and the reaction mixture was stirred at 0° C. for 0.5 hour. MeI (197 mg, 1.39 mmol) was added to the reaction mixture and stirred at 20° C. for 2 hours to give yellow solution. The reaction mixture was added into 0.3N aqueous HCl (20 mL) at 0° C. and basified with saturated aqueous NaHCO₃ to pH=8, extracted with DCM (20 mL×2). The combined organic layer was washed with brine (30 mL), dried over anhydrous Na₂SO₄, filtered and concentrated. The residue was purified by flash silica gel chromatography (ISCO®; 4 g SepaFlash®Silica Flash Column, Eluent of ˜50% Ethyl acetate/Petroleum ether gradient @35 mL/min) and then further purified by prep-HPLC (0.225% FA as an additive), then lyophilized to give Compound 60 (9 mg, yield: 17%) as a yellow solid.

¹H NMR (400 MHz, DMSO-d₆) δ 1.59-1.76 (2H, m), 1.84-1.97 (2H, m), 2.97-3.15 (1H, m), 3.42-3.54 (2H, m), 3.75-3.88 (2H, m), 3.96 (3H, s), 7.17 (1H, d, J=3.2 Hz), 7.56 (1H, d, J=3.2 Hz), 7.82 (1H, dd, J=8.4, 1.2 Hz), 8.22 (1H, d, J=8.8 Hz), 8.54-8.61 (2H, m), 8.94 (1H, s).

### Example 37. Preparation of Compound 61

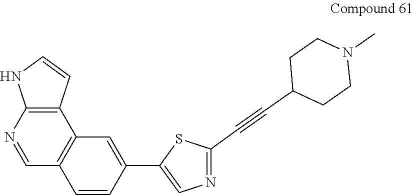 [SMILES: CN1CCC(C#Cc2ncc(-c3ccc4cnc5[nH]ccc5c4c3)s2)CC1]

### Step 1: Preparation of tert-butyl 4-((5-bromothiazol-2-yl)ethynyl)piperidine-1-carboxylate

A mixture of impure 5-bromo-2-iodothiazole (300 mg), tert-butyl 4-ethynylpiperidine-1-carboxylate (144 mg, 0.690 mmol), CuI (26 mg, 0.14 mmol), TEA (349 mg, 3.45 mmol) and Pd(PPh₃)₂Cl₂ (48 mg, 0.069 mmol) in THF (15 mL) was degassed and purged with N₂ for 3 times, then the mixture was stirred at 40° C. for 16 hours under N₂ atmosphere. The mixture turned into yellow suspension. The reaction mixture was concentrated to give a residue. The residue was purified by flash silica gel chromatography (ISCO®; 4 g SepaFlash® Silica Flash Column, Eluent of ˜6% Ethyl acetate/Petroleum ether gradient @30 mL/min) to give impure tert-butyl 4-((5-bromothiazol-2-yl)ethynyl)piperidine-1-carboxylate (100 mg, contains impurities) as yellow oil.

### Step 2: Preparation of tert-butyl 4-((5-(3H-pyrrolo[2,3-c]isoquinolin-8-yl)thiazol-2-yl)ethynyl)piperidine-1-carboxylate

To a solution of Int-23 (350 mg, crude) and tert-butyl 4-((5-bromothiazol-2-yl)ethynyl)piperidine-1-carboxylate (574 mg) in 1, 4-dioxane (6 mL) and H₂O (1.2 mL) was added Pd(dtbpf)Cl₂ (78 mg, 0.12 mmol) and Na₂CO₃ (315 mg, 2.97 mmol) under N₂ atmosphere, the mixture was stirred at 100° C. for 12 hours under N₂ atmosphere to give brown suspension. The reaction mixture was concentrated. The residue was purified by flash silica gel chromatography (ISCO®; 12 g SepaFlash® Silica Flash Column, Eluent of ˜48% Ethyl acetate/Petroleum ether gradient @40 mL/min) to give tert-butyl 4-((5-(3H-pyrrolo[2,3-c]isoquinolin-8-yl)thiazol-2-yl)ethynyl)piperidine-1-carboxylate (80 mg, yield: 51% for two steps) as a yellow solid.

### Step 3: Preparation of 2-(piperidin-4-ylethynyl)-5-(3H-pyrrolo[2,3-c]isoquinolin-8-yl)thiazole

To a solution of tert-butyl 4-((5-(3H-pyrrolo[2,3-c]isoquinolin-8-yl)thiazol-2-yl)ethynyl)piperidine-1-carboxylate (60 mg, 0.13 mmol) in DCM (4 mL) was added TFA (2.16 g, 18.9 mmol) and the reaction mixture was stirred at 20° C. for 2 hours to give yellow solution. The reaction mixture was concentrated to give 2-(piperidin-4-ylethynyl)-5-(3H-pyrrolo[2,3-c]isoquinolin-8-yl)thiazole (90 mg, crude, TFA salt) as a yellow solid which was used into the next step without further purification.

### Step 4: Preparation of 2-((1-methylpiperidin-4-yl)ethynyl)-5-(3H-pyrrolo[2,3-c]isoquinolin-8-yl)thiazole (Compound 61)

A solution of 2-(piperidin-4-ylethynyl)-5-(3H-pyrrolo[2,3-c]isoquinolin-8-yl)thiazole (90 mg, crude, TFA salt) in MeOH (3 mL) was neutralized with DIPEA to pH=7, then 37% aqueous HCHO (124 mg, 1.52 mmol) and HOAc (34 mg, 0.57 mmol) were added to the reaction mixture and the reaction mixture was stirred at 20° C. for 0.5 hour. NaBH₃CN (36 mg, 0.57 mmol) was added to the reaction mixture and stirred at 20° C. for 1.5 hours to give yellow solution. The reaction mixture was concentrated and the residue was purified by prep-HPLC (0.225% FA as an additive), then lyophilized to give Compound 61 (19 mg, yield: 41% for two steps) as a yellow solid.

¹H NMR (400 MHz, DMSO-d₆) δ 1.60-1.75 (2H, m), 1.84-1.96 (2H, m), 2.03-2.21 (5H, m), 2.57-2.68 (2H, m), 2.70-2.88 (1H, m), 7.12-7.19 (1H, m), 7.51 (1H, t, J=2.8 Hz), 7.81 (1H, dd, J=8.4, 1.6 Hz), 8.19 (1H, d, J=8.4 Hz), 8.54 (1H, s), 8.58 (1H, s), 8.90 (1H, s), 12.06 (1H, brs).

### Example 38. Preparation of Compound 62

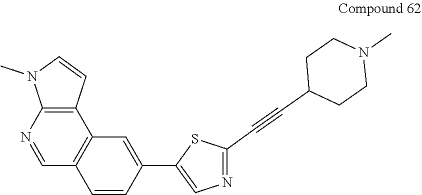

### Step 1: Preparation of tert-butyl 4-((5-(3-methyl-3H-pyrrolo[2,3-c]isoquinolin-8-yl)thiazol-2-yl)ethynyl)piperidine-1-carboxylate

To a solution of tert-butyl 4-((5-(3H-pyrrolo[2,3-c]isoquinolin-8-yl)thiazol-2-yl)ethynyl)piperidine-1-carboxylate (165 mg, 0.359 mmol) in DMF (2 mL) was added NaH (58 mg, 1.4 mmol, 60% dispersion in mineral oil) at 0° C. and the reaction mixture was stirred at 0° C. for 0.5 hour. MeI (511 mg, 3.60 mmol) was added to the reaction mixture and the reaction mixture was stirred at 20° C. for 1.5 hours to give yellow solution. The reaction mixture was added into 0.2N aqueous HCl (30 mL) at 0° C., then basified with saturated aqueous NaHCO₃ to pH=8 and extracted with DCM (40 mL). The organic layer was washed with H₂O (40 mL), dried over anhydrous Na₂SO₄, filtered and concentrated. The residue was purified by flash silica gel chromatography (ISCO®; 4 g SepaFlash® Silica Flash Column, Eluent of ˜-50% Ethyl acetate/Petroleum ether gradient @40 mL/min) to give tert-butyl 4-((5-(3-methyl-3H-pyrrolo[2,3-c]isoquinolin-8-yl)thiazol-2-yl)ethynyl)piperidine-1-carboxylate (55 mg, impure) as a yellow solid.

### Step 2: Preparation of 5-(3-methyl-3H-pyrrolo[2,3-c]isoquinolin-8-yl)-2-(piperidin-4-ylethynyl)thiazole

To a solution of tert-butyl 4-((5-(3-methyl-3H-pyrrolo[2,3-c]isoquinolin-8-yl)thiazol-2-yl)ethynyl)piperidine-1-carboxylate (55 mg, impure) in DCM (3 mL) was added TFA (3 mL) and the reaction mixture was stirred at 20° C. for 2 hours to give yellow solution. The reaction mixture was concentrated to give 5-(3-methyl-3H-pyrrolo[2,3-c]isoquinolin-8-yl)-2-(piperidin-4-ylethynyl)thiazole (80 mg, crude, TFA salt) as a yellow solid, which was used into the next step without further purification.

### Step 3: Preparation of 5-(3-methyl-3H-pyrrolo[2,3-c]isoquinolin-8-yl)-2-((1-methylpiperidin-4-yl)ethynyl)thiazole (Compound 62)

A solution of 5-(3-methyl-3H-pyrrolo[2,3-c]isoquinolin-8-yl)-2-(piperidin-4-ylethynyl)thiazole (80 mg, crude, TFA salt) in MeOH (2 mL) was neutralized with DIPEA to pH=7. 37% aqueous HCHO (107 mg, 1.32 mmol) and HOAc (30 mg, 0.49 mmol) were added to the reaction mixture and stirred at 20° C. for 0.5 hour. NaBH₃CN (31 mg, 0.49 mmol) was added to the reaction mixture and stirred at 20° C. for another 1.5 hours to give yellow solution. The reaction mixture was concentrated and the residue was purified by prep-HPLC (0.05% NH₃H₂O+10 mM NH₄HCO₃ as an additive), then purified by SFC (column: DAICEL CHIRALPAK IA (250 mm*30 mm, 10 um); mobile phase: [0.1% NH₃H₂O EtOH]; B %: 60%-60%), further purified by prep-HPLC (0.225% FA as an additive) and lyophilized to give Compound 62 (3 mg, yield: 2% for three steps) as a yellow solid.

¹H NMR (400 MHz, DMSO-d₆) δ 1.61-1.75 (2H, m), 1.86-1.96 (2H, m), 2.06-2.22 (5H, m), 2.56-2.65 (2H, m), 2.73-2.85 (1H, m), 3.96 (3H, s), 7.17 (1H, d, J=3.2 Hz), 7.56 (1H, d, J=3.2 Hz), 7.82 (1H, dd, J=8.8, 2.0 Hz), 8.19-8.25 (1H, m), 8.54 (1H, s), 8.56-8.59 (1H, m), 8.94 (1H, s).

### Example 39. Preparation of Compound 63

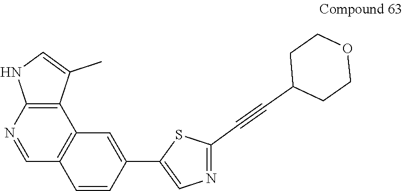 [SMILES: Cc1c[nH]c2ncc3ccc(-c4cnc(C#CC5CCOCC5)s4)cc3c12]

A mixture of Int-24 (150 mg, 0.487 mmol), Int-22 (133 mg, 0.490 mmol), Pd(dtbpf)Cl₂ (32 mg, 0.049 mmol) and Na₂CO₃ (103 mg, 0.980 mmol) in 1, 4-dioxane (4 mL) and H₂O (0.4 mL) was degassed and purged with N₂ for 3 times, and then the mixture was stirred at 90° C. for 1 hour under N₂ atmosphere. The reaction mixture was filtered and the filtrate was concentrated. The residue was purified by silica gel column (PE/EtOAc=1/1), then further purified by prep-HPLC (0.225% FA as an additive) and lyophilized to afford Compound 63 (7.23 mg, yield: 3%) as a yellow solid.

¹H NMR (400 MHz, CDCl₃) δ 1.80-1.90 (2H, m), 1.93-2.03 (2H, m), 2.74 (3H, s), 2.89-3.03 (1H, m), 3.52-3.67 (2H, m), 3.93-4.04 (2H, m), 7.17 (1H, s), 7.69 (1H, dd, J=8.4, 1.6 Hz), 8.09 (1H, d, J=8.4 Hz), 8.14 (1H, s), 8.52 (1H, s), 8.86 (1H, s), 9.14 (1H, brs).

### Example 40. Preparation of Compound 64

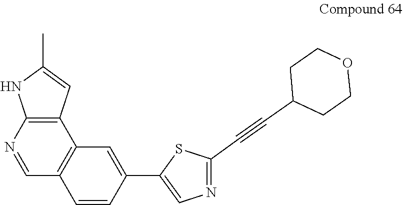 [SMILES: Cc1cc2c(ncc3ccc(-c4cnc(C#CC5CCOCC5)s4)cc32)[nH]1]

A mixture of Int-25 (140 mg, 0.454 mmol), Int-22 (148 mg, 0.545 mmol), Pd(dtbpf)Cl₂ (30 mg, 0.045 mmol) and Na₂CO₃ (96 mg, 0.91 mmol) in 1, 4-dioxane (4 mL) and H₂O (0.4 mL) was degassed and purged with N₂ for 3 times, then the mixture was stirred at 90° C. for 1 hour under N₂ atmosphere. The reaction mixture was concentrated and the residue was purified by silica gel column (EtOAc as eluent), then further purified by prep-HPLC (0.225% FA as an additive) and lyophilized to afford Compound 64 (19.1 mg, yield: 11%) as a yellow solid.

¹H NMR (400 MHz, DMSO-d₆) δ 1.62-1.73 (2H, m), 1.87-1.95 (2H, m), 3.01-3.11 (1H, m), 3.30 (3H, s), 3.44-3.51 (2H, m), 3.79-3.85 (2H, m), 6.84 (1H, d, J=0.8 Hz), 7.77 (1H, dd, J=8.4, 1.6 Hz), 8.15 (1H, d, J=8.4 Hz), 8.47 (1H, s), 8.53 (1H, s), 8.78 (1H, s), 11.88 (1H, brs).

### Example 41. Preparation of Compound 68, 69, and 70

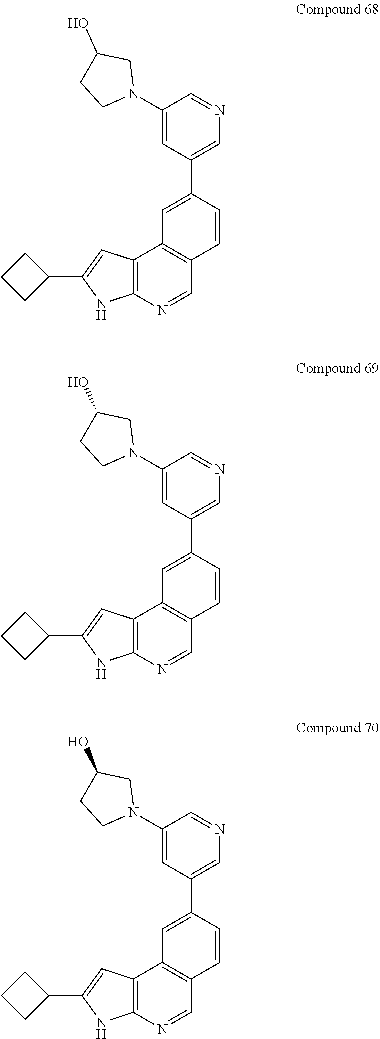 [SMILES: OC1CCN(c2cncc(-c3ccc4cnc5[nH]c(C6CCC6)cc5c4c3)c2)C1 ; O[C@H]1CCN(c2cncc(-c3ccc4cnc5[nH]c(C6CCC6)cc5c4c3)c2)C1 ; O[C@@H]1CCN(c2cncc(-c3ccc4cnc5[nH]c(C6CCC6)cc5c4c3)c2)C1]

### Step 1: Preparation of 6-bromo-4-(cyclobutylethynyl)isoquinolin-3-amine

A solution of Int-1 (1.00 g, 2.87 mmol), Int-26 (523 mg, 3.44 mmol), CuI (109 mg, 0.573 mmol), Pd(PPh₃)₂Cl₂ (201 mg, 0.286 mmol) and TEA (1.45 g, 14.3 mmol) in THF (15 mL) was degassed and purged with N₂ for 3 times, TBAF (5.7 mL, 5.73 mmol, 1M in THF) was added to the reaction mixture and stirred at 70° C. for 1.1 hour. The reaction mixture was concentrated and the residue was purified by Combi Flash (0% to 10% MeOH in DCM) to give 6-bromo-4-(cyclobutylethynyl)isoquinolin-3-amine (400 mg, yield: 40%) as a light brown solid.

### Step 2: Preparation of 8-bromo-2-cyclobutyl-3H-pyrrolo[2,3-c]isoquinoline

To a solution of 6-bromo-4-(cyclobutylethynyl)isoquinolin-3-amine (280 mg, 0.929 mmol) in NMP (5 mL) was added NaH (185 mg, 4.65 mmol, 60% dispersion in mineral oil). The mixture was stirred at 20° C. for 1 hour, then at 100° C. for 12 hours. The reaction mixture was quenched by addition water (20 mL) at 0° C. and extracted with EtOAc (20 mL×3). The combined organic layer was washed with water (10 mL) and brine (10 mL), dried over anhydrous Na₂SO₄, filtered and concentrated. The residue was purified by Combi Flash (0% to 100% EtOAc in PE) to give 8-bromo-2-cyclobutyl-3H-pyrrolo[2,3-c]isoquinoline (150 mg, yield: 53%) as a yellow solid.

¹H NMR (400 MHz, CDCl₃) δ 2.11-2.21 (2H, m), 2.47-2.60 (4H, m), 3.80-3.85 (1H, m), 6.67-6.75 (1H, m), 7.49-7.57 (1H, m), 7.89 (1H, d, J=8.8 Hz), 8.32 (1H, s), 8.77-8.85 (1H, m), 10.80 (1H, s).

### Step 3: Preparation of 8-(5-(3-((tert-butyldimethylsilyl)oxy)pyrrolidin-1-yl)pyridin-3-yl)-2-cyclobutyl-3H-pyrrolo[2,3-c]isoquinoline

A mixture of 8-bromo-2-cyclobutyl-3H-pyrrolo[2,3-c]isoquinoline (120 mg, 0.398 mmol), Int-27 (513 mg, 1.59 mmol), Pd(dppf)Cl₂ (29 mg, 0.040 mmol) and Na₂CO₃ (84 mg, 0.80 mmol) in dioxane (3 mL) and H₂O (0.6 mL) was degassed and purged with N₂ for 3 times, and then the mixture was stirred at 100° C. for 1 hour under N₂ atmosphere. The reaction mixture was concentrated and the residue was purified by Combi Flash (0% to 100% EtOAc in PE) to give 8-(5-(3-((tert-butyldimethylsilyl)oxy)pyrrolidin-1-yl)pyridin-3-yl)-2-cyclobutyl-3H-pyrrolo[2,3-c]isoquinoline (130 mg, yield: 55%) as a yellow solid.

### Step 4: Preparation of Compound 68, 69, and 70

To a solution of 8-(5-(3-((tert-butyldimethylsilyl)oxy)pyrrolidin-1-yl)pyridin-3-yl)-2-cyclobutyl-3H-pyrrolo[2,3-c]isoquinoline (120 mg, 0.240 mmol) in absolute MeOH (2 mL) was added 6N aqueous HCl (2 mL) at 20° C. The mixture was stirred at 20° C. for 1 hour. The reaction mixture was concentrated and the residue was basified with 2N aqueous NaOH to pH =8, then extracted with DCM (20 mL×3). The combined organic layer was washed with brine (20 mL), dried over anhydrous Na₂SO₄, filtered and concentrated. The residue was purified by prep-HPLC (0.225% FA as an additive) to give Compound 68 (30 mg) as a yellow solid. It was submitted for SFC separation (SFC condition: Daicel Chiralpak IE (250 mm×30 mm, 10 um); mobile phase: [0.1% NH₃H₂O in EtOH]) then lyophilized to give Compound 69 (14.47 mg, yield: 15%, ee=100%) as a yellow solid and Compound 70 (14.03 mg, yield: 14%, ee=100%) as a yellow solid.

Compound 69: ¹H NMR (400 MHz, DMSO-d₆) δ 1.85-2.01 (2H, m), 2.01-2.16 (2H, m), 2.31-2.40 (4H, m), 3.20-3.30 (1H, m), 3.41-3.52 (2H, m), 3.50-3.60 (1H, m), 3.65-3.82 (1H, m), 4.45-4.50 (1H, m), 4.92-5.14 (1H, m), 6.99 (1H, s), 7.26 (1H, t, J=2.0 Hz), 7.81 (1H, dd, J=8.8, 2.0 Hz), 7.98 (1H, d, J=2.4 Hz), 8.17 (1H, d, J=8.8 Hz), 8.33 (1H, d, J=2.0 Hz), 8.54 (1H, s), 8.83 (1H, s), 11.87 (1H, brs).

Compound 70: ¹H NMR (400 MHz, DMSO-d₆) δ 1.86-2.00 (2H, m), 2.00-2.12 (2H, m), 2.31-2.40 (4H, m), 3.20-3.30 (1H, m), 3.41-3.52 (2H, m), 3.50-3.60 (1H, m), 3.65-3.82 (1H, m), 4.45-4.50 (1H, m), 4.92-5.14 (1H, m), 6.99 (1H, d, J=2.0 Hz), 7.26 (1H, t, J=2.0 Hz), 7.81 (1H, dd, J=8.4, 1.6 Hz), 7.98 (1H, s), 8.17 (1H, d, J=8.8 Hz), 8.33 (1H, s), 8.54 (1H, s), 8.83 (1H, s), 11.87 (1H, s).

### Example 42. Preparation of Compound 71

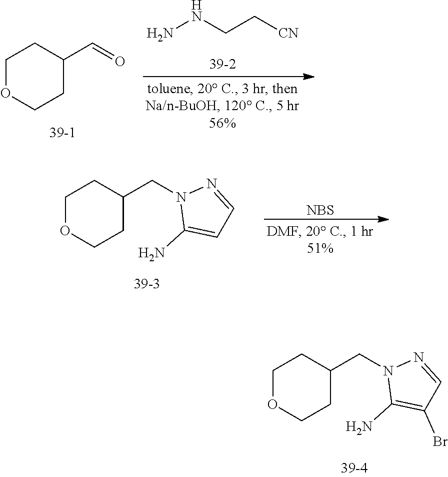 [SMILES: N#CCCNN ; O=CC1CCOCC1 ; Nc1ccnn1CC1CCOCC1 ; "NBS" ; Nc1c(Br)cnn1CC1CCOCC1]

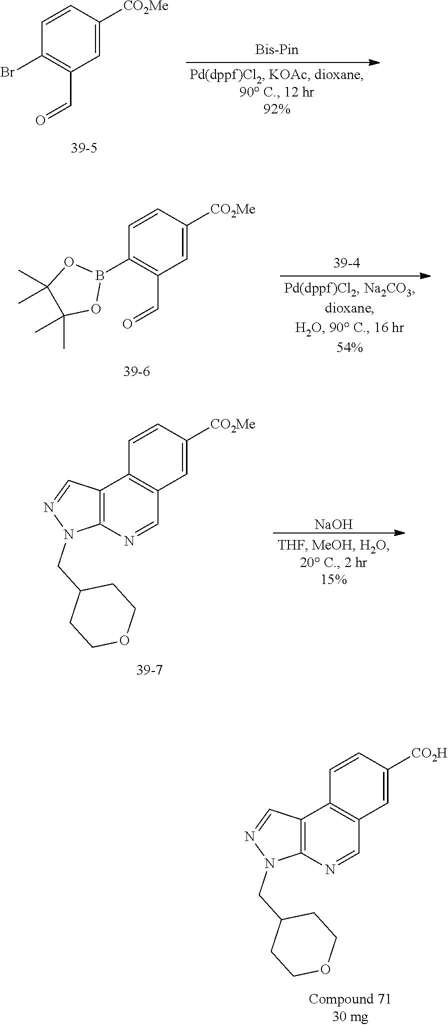 [SMILES: COC(=O)c1ccc(Br)c(C=O)c1 ; COC(=O)c1ccc(B2OC(C)(C)C(C)(C)O2)c(C=O)c1 ; COC(=O)c1ccc2c(cnc3c2cnn3CC2CCOCC2)c1 ; "NaOH" ; O=C(O)c1ccc2c(cnc3c2cnn3CC2CCOCC2)c1]

### Preparation of Compound 39-3

To a mixture of compound 39-1 (500 mg, 5.87 mmol) in toluene (5 mL) was added a solution of compound 39-2 (671 mg, 5.87 mmol) in toluene (5 mL) at 20° C., then the mixture was stirred at 20° C. for 3 hours. The reaction mixture was concentrated and the residue was added into a solution of sodium butoxide in n-BuOH, which was prepared by Na (410 mg, 17.83 mmol) reacted with n-BuOH (10 mL) and the reaction mixture was stirred at 120° C. for 5 hours. The reaction mixture was quenched with isopropanol (20 mL) and concentrated. The residue was purified by silica gel column (DCM/MeOH=10/1) to afford compound 39-3 (600 mg, yield: 56%) as black-brown oil.

¹H NMR (400 MHz, CDCl₃) δ 1.31-1.46 (2H, m), 1.47-1.62 (2H, m), 2.13-2.27 (1H, m), 3.34-3.41 (2H, m), 3.83 (2H, d, J=7.2 Hz), 3.95-4.05 (2H, m), 5.56 (1H, d, J=2.0 Hz), 7.29 (1H, d, J=2.0 Hz).

### Preparation of Compound 39-4

To a solution of compound 39-3 (500 mg, 2.76 mmol) in DMF (5 mL) was added NBS (540 mg, 3.03 mmol) at 20° C., then stirred at 20° C. for 1 hour. The reaction mixture was concentrated and the residue was diluted with H₂O (20 mL), then extracted with EtOAc (40 mL×3). The combined organic layer was washed with brine (40 mL), dried over anhydrous Na₂SO₄, filtered and concentrated. The residue was purified by silica gel column (EtOAc as eluent) to afford compound 39-4 (440 mg, yield: 51%) as a red solid.

¹H NMR (400 MHz, DMSO-d₆) δ 1.15-1.29 (2H, m), 1.35-1.40 (2H, m), 1.93-2.06 (1H, m), 3.16-3.26 (2H, m), 3.69-3.89 (4H, m), 5.35 (2H, brs), 7.14 (1H, s).

### Preparation of Compound 39-6

A mixture of compound 39-5 (800 mg, 3.29 mmol), Bis-Pin (1.25 g, 4.94 mmol), Pd(dppf)Cl₂ (241 mg, 0.330 mmol) and KOAc (969 mg, 9.87 mmol) in 1, 4-dioxane (10 mL) was degassed and purged with N₂ for 3 times, then the mixture was stirred at 90° C. for 16 hours under N₂ atmosphere. The reaction mixture was filtered and the filtrate was concentrated. The residue was purified by silica gel column (PE/EtOAc=2/1) to afford compound 39-6 (890 mg, yield: 92%) as a white solid.

¹H NMR (400 MHz, CDCl₃) δ 1.42 (12H, s), 3.97 (3H, s), 7.91 (1H, d, J=7.6 Hz), 8.24 (1H, dd, J=7.6, 1.6 Hz), 8.57 (1H, d, J=1.6 Hz), 10.52 (1H, s).

### Preparation of Compound 39-7

A mixture of compound 39-6 (250 mg, 0.862 mmol), compound 39-4 (224 mg, 0.862 mmol), Pd(dppf)Cl₂ (63 mg, 0.086 mmol), Na₂CO₃ (274 mg, 2.59 mmol) in 1, 4-dioxane (4 mL) and H₂O (0.4 mL) was degassed and purged with N₂ for 3 times, then the mixture was stirred at 90° C. for 16 hours under N₂ atmosphere. The reaction mixture was concentrated and the residue was purified by silica gel column (EtOAc as eluent) to afford compound 39-7 (150 mg, yield: 54%) as a yellow solid.

¹H NMR (400 MHz, CDCl₃) δ 1.46-1.57 (4H, m), 2.34-2.46 (1H, m), 3.32-3.43 (2H, m), 3.90-4.00 (2H, m), 4.03 (3H, s), 4.55 (2H, d, J=7.2 Hz), 8.24 (1H, d, J=8.8 Hz), 8.39-8.48 (2H, m), 8.85 (1H, d, J=1.2 Hz), 9.14 (1H, s).

### Preparation of Compound 71

A solution of compound 39-7 (150 mg, 0.461 mmol) in THF (1.6 mL), MeOH (1.6 mL) and H₂O (0.8 mL) was added NaOH (74 mg, 1.8 mmol), the reaction mixture was stirred at 20° C. for 2 hours. The reaction mixture was acidified with FA to pH=3 and filtered. The crude product was washed with water (1 mL×3), then triturated with MeOH (3 mL) and lyophilized to afford Compound 71 (21.08 mg, yield: 15%) as a white solid.

¹H NMR (400 MHz, DMSO-d₆) δ 1.24-1.45 (4H, m), 2.20-2.32 (1H, m), 3.22-3.27 (2H, m), 3.75-3.85 (2H, m), 4.49 (2H, d, J=7.2 Hz), 8.37 (1H, dd, J=8.8, 1.6 Hz), 8.46 (1H, d, J=8.4 Hz), 8.70 (1H, s), 8.92 (1H, d, J=1.2 Hz), 9.37 (1H, s).

### Example 43. Preparation of Compound 72

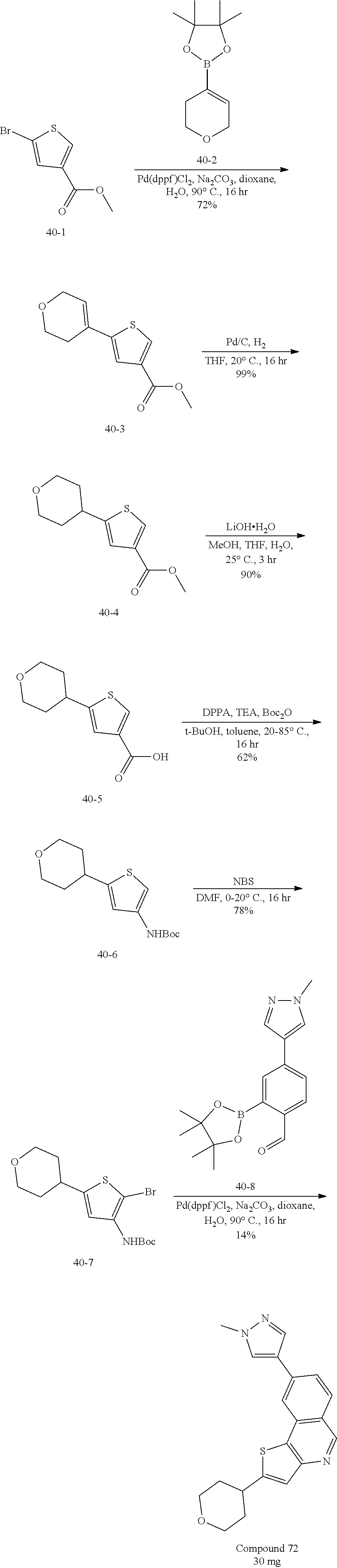

### Preparation of Compound 40-3

A mixture of compound 40-1 (3.00 g, 13.6 mmol), compound 40-2 (3.42 g, 16.3 mmol), Pd(dppf)Cl₂ (993 mg, 1.36 mmol) and Na₂CO₃ (2.88 g, 27.1 mmol) in 1, 4-dioxane (30 mL) and H₂O (3 mL) was degassed and purged with N₂ for 3 times, and then the mixture was stirred at 90° C. for 16 hours under N₂ atmosphere. The reaction mixture was concentrated and the residue was purified by silica gel column (0% to 10% EtOAc in PE) to give compound 40-3 (2.20 g, yield: 72%) as a yellow solid.

¹H NMR (400 MHz, DMSO-d₆) δ 2.42-2.49 (2H, m), 3.76-3.83 (5H, m), 4.17-4.22 (2H, m), 6.24-6.26 (1H, m), 7.37 (1H, s), 8.22 (1H, s).

### Preparation of Compound 40-4

To a solution of compound 40-3 (2.20 g, 9.81 mmol) in THF (50 mL) was added Pd/C (300 mg, 10% purity). The mixture was degassed and purged with H₂ for 3 times, and then the mixture was stirred at 20° C. for 16 hours under H₂ atmosphere (15 Psi). The reaction mixture were filtered and the solid was washed with THF (10 mL×3), the filtrate was concentrated to give compound 40-4 (2.20 g, yield: 99%) as a yellow solid.

### Preparation of Compound 40-5

To a solution of compound 40-4 (2.20 g, 9.72 mmol) in THF (10 mL) and CH₃OH (10 mL) was added LiOH·H₂O (4.08 g, 97.2 mmol) and H₂O (5 mL). The mixture was stirred at 25° C. for 3 hours. The reaction mixture was concentrated and the residue was acidified with 1N aqueous HCl to pH=5 and filtered. The solid was washed with water (5 mL×2) and dried to give compound 40-5 (1.85 g, yield: 90%) as a yellow solid.

### Preparation of Compound 40-6

To a solution of compound 40-5 (850 mg, 4.00 mmol) in toluene (25 mL) was added TEA (1.22 g, 12.0 mmol) and DPPA (3.31 g, 12.0 mmol). The mixture was stirred at 20° C. for 1 hour. Then the Boc₂O (3.50 g, 16.0 mmol) and t-BuOH (25 mL) were added to the mixture and stirred at 85° C. for 15 hours. The reaction mixture was concentrated and the residue was diluted with H₂O (50 mL) and extracted with EtOAc (80 mL×3). The combined organic layers were washed with brine (100 mL), dried over anhydrous Na₂SO₄, filtered and concentrated. The residue was purified by silica gel column (0% to 11% EtOAc in PE) to give compound 40-6 (700 mg, yield: 62%) as yellow oil.

### Preparation of Compound 40-7

To a solution of compound 40-6 (200 mg, 0.705 mmol) in DMF (5 mL) was added NBS (126 mg, 0.705 mmol) at 0° C. The mixture was stirred at 20° C. for 16 hours. The reaction mixture was poured into water (20 mL) and filtered. The solid was washed with water (10 mL×2) and dried. The residue was purified by silica gel column (0% to 5% EtOAc in PE) to give compound 40-7 (200 mg, yield: 78%) as a yellow solid.

¹H NMR (400 MHz, DMSO-d₆) δ 1.45 (9H, s), 1.53-1.62 (2H, m), 1.79-1.88 (2H, m), 2.94-3.06 (1H, m), 3.36-3.46 (2H, m), 3.84-3.95 (2H, m), 6.91 (1H, s), 8.73 (1H, brs).

### Preparation of Compound 72

A mixture of compound 40-7 (200 mg, 0.552 mmol), compound 40-8 (207 mg, 0.662 mmol), Pd(dppf)Cl₂ (40 mg, 0.055 mmol) and Na₂CO₃ (117 mg, 1.10 mmol) in dioxane (8 mL) and H₂O (1 mL) was degassed and purged with N₂ for 3 times, and then the mixture was stirred at 90° C. for 16 hours under N₂ atmosphere. The reaction mixture was suspended in CH₃OH (20 mL) and filtered. The filtrate was concentrated and the residue was purified by prep-HPLC (0.225% o FA as an additive), then lyophilized to afford Compound 72 (27.91 mg, yield: 14% o) as a yellow solid

¹H NMR (400 MHz, DMSO-d₆) δ 2 1.73-1.87 (2H, m), 1.99-2.11 (2H, n), 3.25-3.28 (1H, m), 3.49-3.57 (2H, m), 3.92 (3H, s), 3.96-4.03 (2H, n), 7.48 (1H, s), 7.92 (1H, dd, J=8.4, 1.6 5 Hz), 8.13-8.26 (3H, in), 8.50 (1H, s), 9.16 (1H, s).

The following compounds were synthesized analogously to Compound 72

| Comp. | | | |
|---|---|---|---|
| No | Structure | Name | ¹H NMR (400 MHz) |
| 73 | 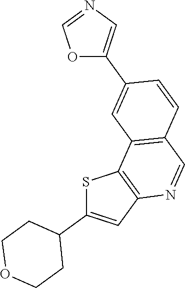 [SMILES: c1ncc(-c2ccc3cnc4cc(C5CCOCC5)sc4c3c2)o1] | 5-(2-(tetrahydro- 2H-pyran-4- yl)thieno[3,2- c]isoquinolin-8- yl)oxazole | DMSO-d₆; δ 1.73-1.85 (2H, m), 1.99- 2.09 (2H, m), 3.27-3.31 (1H, m), 3.45-3.58 (2H, m), 3.91-4.05 (2H, m), 7.53 (1H, s), 8.04 (1H, dd, J = 8.4, 1.2 Hz), 8.09 (1H, s), 8.27-8.40 (2H, m), 8.62 (1H, s), 9.25 (1H, s). |
| 74 | 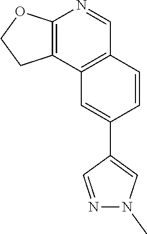 [SMILES: Cn1cc(-c2ccc3cnc4c(c3c2)CCO4)cn1] | 8-(1-methyl-1H- pyrazol-4-yl)-1,2- dihydrofuro[2,3- c]isoquinoline | DMSO-d₆; δ 3.51 (2H, t, J = 8.8 Hz), 3.91 (3H, s), 4.73 (2H, t, J = 8.8 Hz), 7.65 (1H, dd, J = 8.8, 1.6 Hz), 7.83 (1H, s), 8.01 (1H, d, J = 8.4 Hz), 8.10 (1H, s), 8.39 (1H, s), 8.76 (1H, s). |
| 75 | 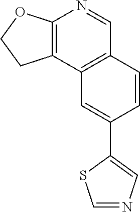 [SMILES: c1ncc(-c2ccc3cnc4c(c3c2)CCO4)s1] | 8-(thiazol-5-yl)-1,2- dihydrofuro[2,3- c]isoquinoline | DMSO-d₆; δ 3.56 (2H, t, J = 8.8 Hz), 4.76 (2H, t, J = 8.8 Hz), 7.77 (1H, dd, J = 8.4, 1.6 Hz), 7.94 (1H, s), 8.12 (1H, d, J = 8.4 Hz), 8.59 (1H, s), 8.87 (1H, s), 9.22 (1H, s). |
| 76 | 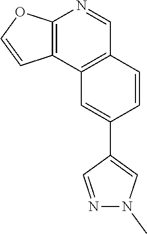 [SMILES: Cn1cc(-c2ccc3cnc4occc4c3c2)cn1] | 8-(1-methyl-1H- pyrazol-4- yl)furo[2,3- c]isoquinoline | DMSO-d₆; δ 3.94 (3H, s), 7.61 (1H, d, J = 2.4 Hz), 7.90 (1H, dd, J = 8.8, 1.6 Hz), 8.13-8.20 (2H, m), 8.23 (1H, d, J = 8.4 Hz), 8.43 (1H, s), 8.55 (1H, s), 8.97 (1H, s). |
| 78 | 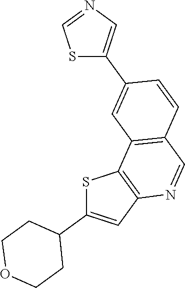 [SMILES: c1ncc(-c2ccc3cnc4cc(C5CCOCC5)sc4c3c2)s1] | 2-(tetrahydro-2H- pyran-4-yl)-8- (thiazol-5- yl)thieno[3,2- c]isoquinoline | DMSO-d₆; δ 1.74-1.87 (2H, m), 2.00- 2.09 (2H, m), 3.27-3.30 (1H, m), 3.48-3.57 (2H, m), 3.95-4.04 (2H, m), 7.53 (1H, s), 8.03 (1H, dd, J = 8.4, 1.6 Hz), 8.25-8.36 (2H, m), 8.68 (1H, s), 9.24-9.27 (2H, m). |
| 79 | 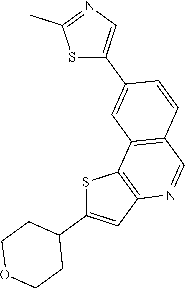 [SMILES: Cc1ncc(-c2ccc3cnc4cc(C5CCOCC5)sc4c3c2)s1] | 8-(2-methylthiazol- 5-yl)-2-(tetrahydro- 2H-pyran-4- yl)thieno[3,2- c]isoquinoline | DMSO-d₆; δ 1.73-1.87 (2H, m), 1.99- 2.10 (2H, m), 2.75 (3H, s), 3.27-3.30 (1H, m), 3.46-3.58 (2H, m), 3.95- 4.05 (2H, m), 7.52 (1H, s), 7.96 (1H, dd, J = 8.4, 1.6 Hz), 8.20 (1H, s), 8.28 (1H, d, J = 8.4 Hz), 8.41 (1H, s), 9.23 (1H, s). |
| 86 | 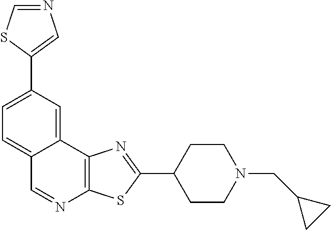 [SMILES: c1ncc(-c2ccc3cnc4sc(C5CCN(CC6CC6)CC5)nc4c3c2)s1] | 2-(1- (cyclopropylmethyl) piperidin-4-yl)-8- (thiazol-5- yl)thiazolo[5,4- c]isoquinoline | CD₃OD; δ 0.01-0.05 (2H, m), 0.33- 0.44 (2H, m), 0.69-0.81 (1H, m), 1.80-1.96 (2H, m), 2.05-2.15 (4H, m), 2.17 (2H, d, J = 6.4 Hz), 3.03- 3.08 (2H, m), 3.11-3.12 (1H, m), 7.88 (1H, dd, J = 8.4, 2.0 Hz), 8.08 (1H, d, J = 8.8 Hz), 8.28 (1H, s), 8.64 (1H, s), 8.91 (1H, s), 8.96 (1H, s). |

### Example 44. Preparation of Compound 77

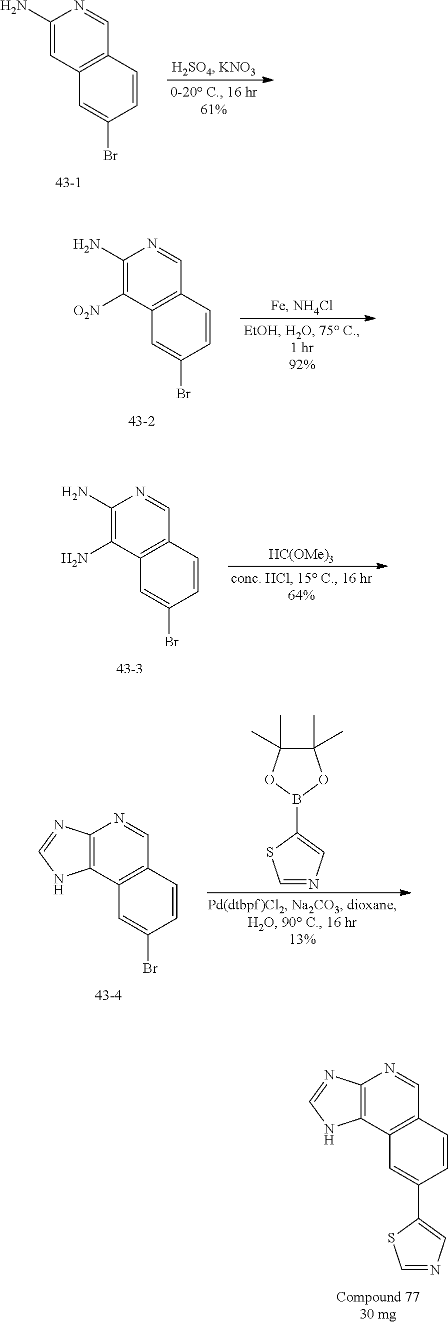

### Preparation of Compound 43-2

To a solution of compound 43-1 (300 mg, 1.34 mmol) in conc. H₂SO₄ (5 mL) was added KNO₃ (136 mg, 1.34 mmol) at 0′° C. The mixture was stirred at 20° C. for 16 hours. The reaction mixture was poured into ice-water (30 mL) and filtered. The solid was washed with water (10 mL×2) and dried. The crude product was triturated with CH₃OH (5 mL) to give compound 43-2 (220 mg, yield: 61%) as a yellow solid.

### Preparation of Compound 43-3

To a solution of compound 43-2 (220 mg, 0.821 mmol) in EtOH (4 mL) and H₂O (4 mL) was added Fe powder (183 mg, 3.28 mmol) and NH₄Cl (176 mg, 3.28 mmol). The mixture was stirred at 75° C. for 1 hour. The reaction mixture was suspended in CH₃OH (20 mL) and filtered. The filtrate was concentrated to give compound 43-3 (180 mg, yield: 92%) as a yellow solid.

### Preparation of Compound 43-4

To a solution of compound 43-3 (180 mg, 0.756 mmol) in trimethoxymethane (4.59 g, 43.2 mmol) was added ION aqueous HCl (0.15 mL). The mixture was stirred at 15° C. for 16 hours. The reaction mixture was concentrated and the residue was basified with saturated aqueous Na₂CO₃ to pH=10, then extracted with EtOAc (15 mL×3). The combined organic layer was washed brine (15 mL), dried over anhydrous Na₂SO₄ and concentrated. The residue was purified by silica gel column (0% to 5% CH₃OH in DCM) to give compound 43-4 (120 mg, yield: 64%) as a yellow solid.

¹H NMR (400 MHz, DMSO-d₆) δ 7.77 (1H, dd, J=8.8, 2.0 Hz), 8.22 (1H, d, J=8.8 Hz), 8.47 (1H, s), 8.58 (1H, s), 9.11 (1H, s), 13.49 (1H, brs).

### Preparation of Compound 77

A mixture of compound 43-4 (100 mg, 0.403 mmol), 5-(4,4,5,5-tetramethyl-1,3,2-dioxaborolan-2-yl)thiazole (102 mg, 0.483 mmol), Pd(dtbpf)Cl₂(26 mg, 0.040 mmol) and Na₂CO₃ (85 mg, 0.81 mmol) in 1, 4-dioxane (4 mL) and H₂O (0.5 mL) was degassed and purged with N₂ for 3 times, and then the mixture was stirred at 90° C. for 16 hours under N₂ atmosphere. The reaction mixture was concentrated and the residue was purified by silica gel column (0% to 5% CH₃OH in DCM), then further purified by prep-HPLC (0.225% FA as an additive) and lyophilized to afford Compound 77 (15.07 mg, yield: 13%) as a white solid.

¹H NMR (400 MHz, DMSO-d₆) δ 7.97-8.09 (1H, m), 8.32 (1H, d, J=8.8 Hz), 8.46 (1H, s), 8.50-8.70 (2H, m), 9.09 (1H, s), 9.24 (1H, s), 13.50 (1H, brs).

### Example 45. Preparation of Compound 80

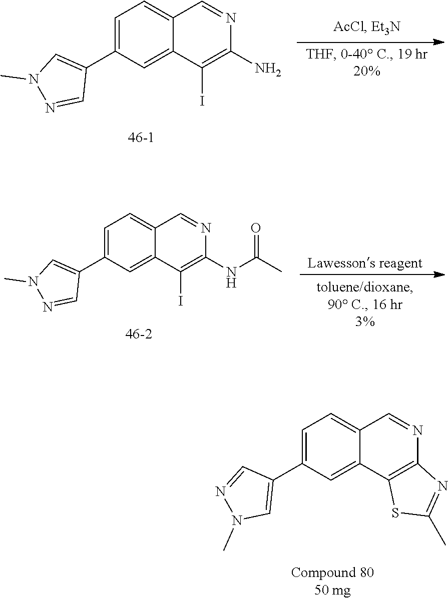 [SMILES: Cn1cc(-c2ccc3cnc(N)c(I)c3c2)cn1 ; CC(=O)Nc1ncc2ccc(-c3cnn(C)c3)cc2c1I ; Cc1nc2ncc3ccc(-c4cnn(C)c4)cc3c2s1]

### Preparation of Compound 46-2

To a solution of compound 46-1 (500 mg, 1.43 mmol) in THF (12 mL) was added AcCl (123 mg, 1.57 mmol) and TEA (159 mg, 1.57 mmol) at 0° C. The resulting mixture was stirred at 25° C. for 3 hours and heated at 40° C. for 16 hours under N₂ atmosphere. The reaction mixture was diluted with H₂O (40 mL) and extracted with EtOAc (40 mL×3). The combined organic layers were washed with H₂O (15 mL) and brine (15 mL), dried over anhydrous Na₂SO₄, filtered and concentrated. The residue was purified by silica gel column (PE/EtOAc=1/1) to afford compound 46-2 (130 mg, yield: 20%) as a yellow solid.

¹H NMR (400 MHz, DMSO-d₆) δ 2.06 (3H, s), 3.93 (3H, s), 7.97 (1H, dd, J=8.4, 1.6 Hz), 8.05-8.10 (2H, m), 8.12 (1H, d, J=8.4 Hz), 8.44 (1H, s), 9.06 (1H, s).

### Preparation of Compound 80

A mixture of compound 46-2 (100 mg, 0.255 mmol) and Lawesson's reagent (103 mg, 0.255 mmol) in toluene (6 mL) and 1, 4-dioxane (6 mL) was stirred at 90° C. for 16 hours under N₂ atmosphere. The reaction mixture was cooled to 25° C. and basified with saturated aqueous NaHCO₃ to pH=8, then diluted with H₂O (30 mL) and extracted with EtOAc (30 mL×3). The combined organic layers were dried over anhydrous Na₂SO₄, filtered and concentrated. The residue was purified by prep-HPLC (0.225% FA as an additive) and lyophilized to afford Compound 80 (2.41 mg, yield: 3%) as a yellow solid.

¹H NMR (400 MHz, DMSO-d₆) δ 2.92 (3H, s), 3.92 (3H, s), 7.98 (1H, dd, J=8.8, 1.6 Hz), 8.20 (1H, s), 8.22-8.28 (2H, m), 8.50 (1H, s), 9.22 (1H, s).

The following compound was synthesized analogously to Compound 80

| Comp. | | | |
|---|---|---|---|
| No | Structure | Name | ¹H NMR (400 MHz) |
| 81 | 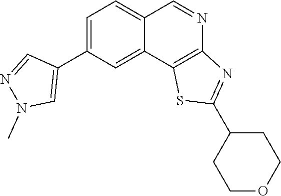 [SMILES: Cn1cc(-c2ccc3cnc4nc(C5CCOCC5)sc4c3c2)cn1] | 8-(1-methyl-1H- pyrazol-4-yl)-2- (tetrahydro-2H- pyran-4- yl)thiazolo[4,5- c]isoquinoline | DMSO-d₆; δ 1.83-1.95 (2H, m), 2.10-2.15 (2H, m), 3.52-3.60 (3H, m), 3.92 (3H, s), 3.95-4.05 (2H, m), 8.00 (1H, dd, J = 8.4, 1.6 Hz), 8.22 (1H, s), 8.26-8.31 (2H, m), 8.52 (1H, s), 9.26 (1H, s) |

### Example 46. Preparation of Compound 82

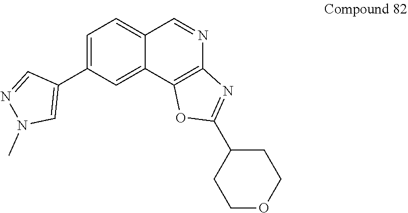 [SMILES: Cn1cc(-c2ccc3cnc4nc(C5CCOCC5)oc4c3c2)cn1]

### Step 1: Preparation of N-(4-iodo-6-(1-methyl-1H-pyrazol-4-yl)isoquinolin-3-yl)tetrahydro-2H-pyran-4-carboxamide

A mixture of Int-5 (500 mg, 1.43 mmol), tetrahydro-2H-pyran-4-carboxylic acid (223 mg, 1.71 mmol), T3P (2.73 g, 4.28 mmol, 50% in EtOAc) in pyridine (10 mL) was stirred at 90° C. for 12 hours. The reaction mixture was quenched with saturated aqueous Na₂CO₃ (25 mL) and extracted with EtOAc (25 mL×3). The combined organic layers were dried over anhydrous Na₂SO₄, filtered and concentrated. The residue was purified by Combi Flash (0% to 100% EtOAc in PE) to give N-(4-iodo-6-(1-methyl-1H-pyrazol-4-yl)isoquinolin-3-yl)tetrahydro-2H-pyran-4-carboxamide (660 mg, yield: 84%) as a brown solid.

### Step 2: Preparation of 8-(1-methyl-1H-pyrazol-4-yl)-2-(tetrahydro-2H-pyran-4-yl)oxazolo[4,5-c]isoquinoline (Compound 82)

A mixture of N-(4-iodo-6-(1-methyl-1H-pyrazol-4-yl)isoquinolin-3-yl)tetrahydro-2H-pyran-4-carboxamide (660 mg, 1.20 mmol), CuI (12 mg, 0.06 mmol), Cs₂CO₃ (781 mg, 2.40 mmol) and 1,10-phenathroline (22 mg, 0.12 mmol) in DMSO (20 mL) was degassed and purged with N₂ for 3 times, then the mixture was stirred at 110° C. for 12 hours under N₂ atmosphere. The reaction mixture was diluted with H₂O (25 mL) and extracted DCM (25 mL×3). The combined organic layers were dried over anhydrous Na₂SO₄, filtered and concentrated. The residue was purified by prep-HPLC (0.05% HCl as an additive) and lyophilized to give Compound 82 (23.92 mg, yield: 6%) as a yellow solid.

¹H NMR (400 MHz, DMSO-d₆) δ 1.90-2.05 (2H, m), 2.10-2.20 (2H, m), 3.40-3.50 (1H, m), 3.55-3.60 (2H, m), 3.93 (3H, s), 3.95-4.05 (2H, m), 7.98 (1H, dd, J=8.8, 1.6 Hz), 8.21 (1H, s), 8.30 (1H, d, J=8.8 Hz), 8.34 (1H, s), 8.53 (1H, s), 9.17 (1H, s).

### Example 47. Preparation of Compound 83

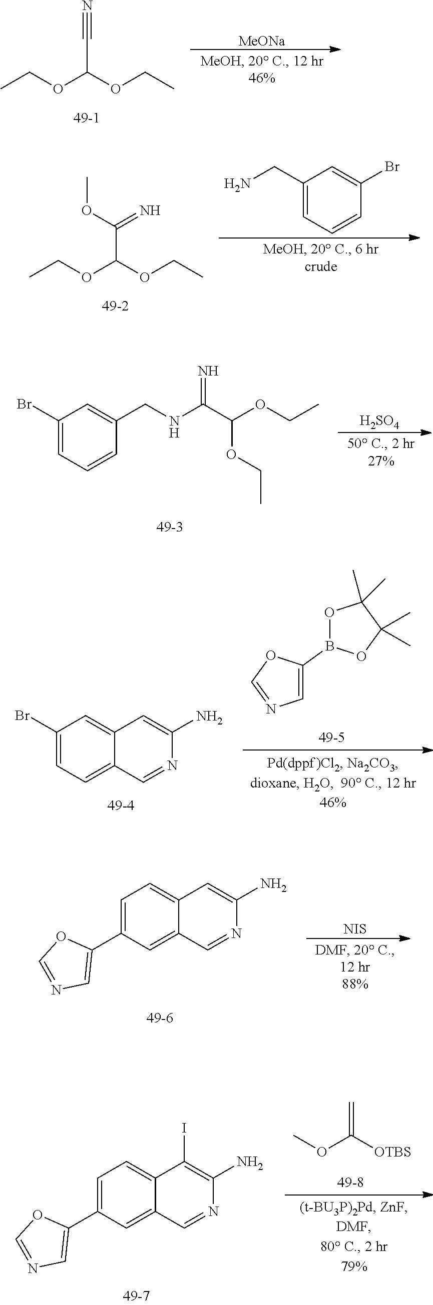

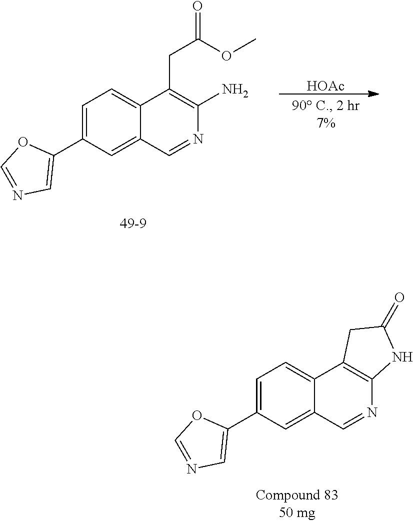 [SMILES: COC(=O)Cc1c(N)ncc2cc(-c3cnco3)ccc12 ; O=C1Cc2c(ncc3cc(-c4cnco4)ccc23)N1 ; "HOAc"]

### Preparation of Compound 49-2

To a solution of compound 49-1 (5.00 g, 38.7 mmol) in MeOH (25 mL) was added MeONa (697 mg, 3.87 mmol) and stirred at 20° C. for 12 hours. The reaction mixture was quenched with dry ice and concentrated. The residue was diluted with H₂O (50 mL) and extracted with DCM (100 mL×3). The combined organic layers were washed with brine (50 mL), dried over anhydrous Na₂SO₄, filtered and concentrated to give compound 49-2 (2.90 g, yield: 46%) as yellow gum.

### Preparation of Compound 49-3

To a solution of (3-bromophenyl)methanamine (1.70 g, 9.14 mmol) in MeOH (20 mL) was added compound 49-2 (2.95 g, 18.3 mmol). The mixture was stirred at 20° C. for 6 hours.

The reaction mixture was concentrated to give compound 49-3 (2.50 g, crude) as yellow oil.

### Preparation of Compound 49-4

A solution of compound 49-3 (2.50 g, 7.93 mmol) in conc. H₂SO₄ (10 mL) was stirred at 50° C. for 2 hours. The reaction mixture was quenched with H₂O (50 mL) at 10° C. and basified with 2N aqueous NaOH to pH=10 and extracted with EtOAc (100 mL×3). The combined organic layers were dried over anhydrous Na₂SO₄, filtered and concentrated. The residue was purified by Combi Flash (0 to 100% EtOAc in PE) to give compound 49-4 (480 mg, yield: 27%) as a brown solid.

¹H NMR (400 MHz, DMSO-d₆) δ 6.09 (2H, brs), 6.61 (1H, s), 7.46-7.56 (2H, m), 8.05 (1H, s), 8.80 (1H, s).

### Preparation of Compound 49-6

A mixture of compound 49-4 (480 mg, 2.15 mmol), compound 49-5 (2.94 g, 15.1 mmol), Pd(dppf)Cl₂ (158 mg, 0.215 mmol) and Na₂CO₃ (456 mg, 4.30 mmol) in 1,4-dioxane (10 mL) and H₂O (1 mL) was degassed and purged with N₂ for 3 times, then the mixture was stirred at 90° C. for 12 hours under N₂ atmosphere. The reaction mixture was diluted with H₂O (25 mL) and extracted with EtOAc (50 mL×3). The combined organic layers were dried over anhydrous Na₂SO₄, filtered and concentrated. The residue was purified by Combi Flash (0 to 40% EtOAc in PE) to give compound 49-6 (210 mg, yield: 46%) as a yellow solid.

### Preparation of Compound 49-7

To a solution of compound 49-6 (200 mg, 0.947 mmol) in DMF (10 mL) was added NIS (235 mg, 1.04 mmol) and stirred at 20° C. for 12 hours. The reaction mixture was concentrated and the residue was diluted with H₂O (25 mL) and extracted with EtOAc (50 mL×3). The combined organic layers were washed with brine (25 mL), dried over anhydrous Na₂SO₄, filtered and concentrated. The residue was purified by Combi Flash (0 to 100% EtOAc in PE) to give compound 49-7 (280 mg, yield: 88%) as a yellow solid.

### Preparation of Compound 49-9

A mixture of compound 49-7 (230 mg, 0.683 mmol), compound 49-8 (514 mg, 2.73 mmol), (t-Bu₃P)₂Pd (70 mg, 0.14 mmol) and ZnF₂ (212 mg, 2.05 mmol) in DMF (10 mL) was degassed and purged with N₂ for 3 times, then the mixture was stirred at 80° C. for 2 hours under N₂ atmosphere. The reaction mixture was concentrated and the residue was diluted with H₂O (25 mL) and extracted with EtOAc (25 mL×3). The combined organic layers were dried over anhydrous Na₂SO₄, filtered and concentrated. The residue was purified by Combi Flash (0 to 30% EtOAc in PE) to give compound 49-9 (70 mg, yield: 25%) as yellow gum.

### Preparation of Compound 83

A mixture of compound 49-4 (70 mg, 0.25 mmol) in HOAc (4 mL) was stirred at 90° C. for 2 hours. The reaction mixture was concentrated and the residue was purified by prep-HPLC (0.225% FA as an additive), then lyophilized to give Compound 83 (4.48 mg, yield: 7%) as an off-white solid.

¹H NMR (400 MHz, DMSO-d₆) δ 3.88 (2H, s), 7.78-7.84 (2H, m), 8.06 (1H, dd, J=8.8, 1.6 Hz), 8.37 (1H, s), 8.53 (1H, s), 9.07 (1H, s), 11.12 (1H, brs).

The following compounds were synthesized analogously to Compound 83

| Comp. | | | |
|---|---|---|---|
| No | Structure | Name | ¹H NMR (400 MHz) |
| 84 | 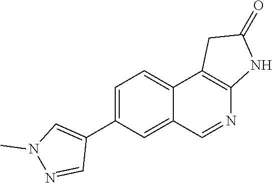 [SMILES: Cn1cc(-c2ccc3c4c(ncc3c2)NC(=O)C4)cn1] | 7-(1-methyl-1H- pyrazol-4-yl)-1,3- dihydro-2H- pyrrolo[2,3- c]isoquinolin-2-one | DMSO-d₆; δ 3.85 (2H, s), 3.91 (3H, s), 7.71 (1H, d, J = 8.8 Hz), 7.96 (1H, dd, J = 8.8, 2.0 Hz), 8.01 (1H, s), 8.20 (1H, s), 8.28 (1H, s), 8.92 (1H, s), 11.00 (1H, brs). |
| 85 | 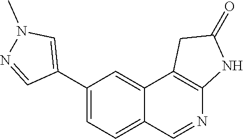 [SMILES: Cn1cc(-c2ccc3cnc4c(c3c2)CC(=O)N4)cn1] | 8-(1-methyl-1H- pyrazol-4-yl)-1,3- dihydro-2H- pyrrolo[2,3- c]isoquinolin-2-one | DMSO-d₆; δ 3.83 (2H, s), 3.91 (3H, s), 7.67 (1H, dd, J = 8.8, 1.6 Hz), 7.84 (1H, s), 8.00 (1H, d, J = 8.8 Hz), 8.08 (1H, s), 8.37 (1H, s), 8.88 (1H, s), 10.96 (1H, brs). |

### Example 48. Preparation of Compound 87

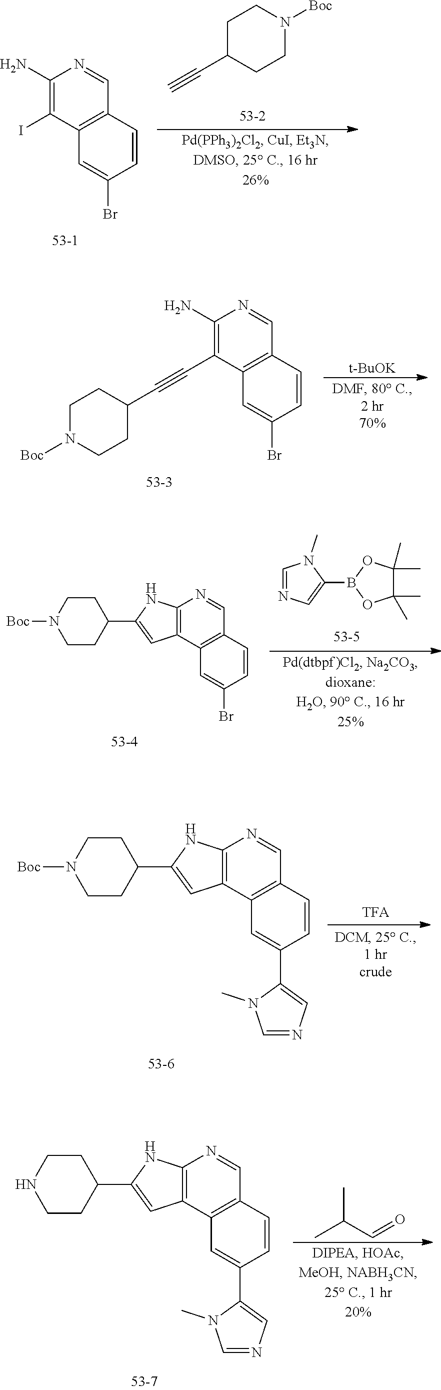

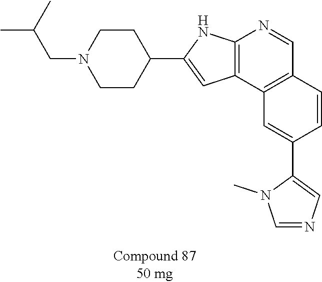 [SMILES: CC(C)CN1CCC(c2cc3c(ncc4ccc(-c5cncn5C)cc43)[nH]2)CC1]

### Preparation of Compound 53-3

A mixture of compound 53-1 (2.50 g, 7.16 mmol), compound 53-2 (1.80 g, 8.60 mmol), Pd(PPh₃)₂Cl₂ (503 mg, 0.716 mmol), CuI (273 mg, 1.43 mmol) and Et₃N (3.62 g, 35.8 mmol) in DMSO (40 mL) was degassed and purged with N₂ for 3 times, then the mixture was stirred at 20° C. for 16 hours under N₂ atmosphere. The residue was diluted with H₂O (50 mL) and extracted with EtOAc (100 mL×3). The combined organic layers were washed with brine (150 mL), dried over anhydrous Na₂SO₄, filtered and concentrated. The residue was purified by Combi Flash (0-30% EtOAc in PE) to give compound 53-3 (800 mg, yield: 26%) as yellow oil.

### Preparation of Compound 53-4

To a solution of compound 53-3 (800 mg, 1.86 mmol) in DMF (15 mL) was added t-BuOK (835 mg, 7.44 mmol). The mixture was stirred at 80° C. for 2 hours. The reaction mixture was diluted with H₂O (50 mL) and extracted with EtOAc (50 mL×3). The combined organic layers were washed with brine (50 mL×3), dried over anhydrous Na₂SO₄, filtered and concentrated. The residue was purified by Combi Flash (0-30% EtOAc in PE) to give compound 53-4 (560 mg, yield: 70%) as a yellow solid.

¹H NMR (400 MHz, DMSO-d₆) δ 1.43 (9H, s), 1.56-1.68 (2H, m), 2.00-2.07 (2H, m), 2.50-2.55 (1H, m), 2.98-3.05 (2H, m), 4.04-4.13 (2H, m), 6.87 (1H, d, J=2.0 Hz), 7.59 (1H, dd, J=8.8, 2.0 Hz), 8.06 (1H, d, J=8.8 Hz), 8.47 (1H, d, J=2.0 Hz), 8.83 (1H, s), 11.98 (1H, brs).

### Preparation of Compound 53-6

A mixture of compound 53-4 (200 mg, 0.465 mmol), compound 53-5 (145 mg, 0.698 mmol), Pd(dtbpf)Cl₂ (31 mg, 0.046 mmol) and Na₂CO₃ (99 mg, 0.93 mmol) in 1,4-dioxane (10 mL) and H₂O (1 mL) was degassed and purged with N₂ for 3 times, then the mixture was stirred at 90° C. for 16 hours under N₂ atmosphere. The reaction mixture was diluted with H₂O (20 mL) and extracted with EtOAc (25 mL×3). The combined organic layers were washed with brine (25 mL), dried over anhydrous Na₂SO₄, filtered and concentrated. The residue was purified by Combi Flash (0-100% EtOAc in PE) to give compound 53-6 (50 mg, yield: 25%) as yellow oil.

### Preparation of Compound 53-7

A solution of compound 53-6 (50 mg, 0.12 mmol) in DCM (2 mL) and TFA (1 mL) was stirred at 25° C. for 1 hour. The reaction mixture was concentrated to give compound 7 (40 mg, crude, TFA salt) as yellow gum.

### Preparation of Compound 87

A solution of compound 53-7 (40 mg, 0.12 mmol, TFA salt) in MeOH (3 mL) was added DIPEA (16 mg, 0.12 mmol) and stirred at 25° C. for 5 minutes, Then HOAc (8 mg, 0.1 mmol) was added and the reaction mixture stirred at 25° C. for 10 minutes. The isobutyraldehyde (26 mg, 0.36 mmol) was added to the reaction mixture and stirred for 0.5 hour. NaBH₃CN (38 mg, 0.60 mmol) was added and stirred at 25° C. for 15 minutes. The reaction mixture was quenched with NH₄Cl (0.5 mL) and concentrated. The residue was purified by prep-HPLC (0.225% FA as an additive) and lyophilized to give Compound 87 (9.48 mg, yield: 20%, FA salt) as a yellow solid.

¹H NMR (400 MHz, DMSO-d₆) δ 0.90 (6H, d, J=6.4 Hz), 1.76-1.90 (3H, m), 2.03-2.23 (6H, m), 2.77-2.87 (1H, m), 2.97-3.08 (2H, m), 3.84 (3H, s), 6.89 (1H, s), 7.28 (1H, s), 7.60 (1H, dd, J=8.4, 0.8 Hz), 7.81 (1H, s), 8.08-8.22 (2H, m), 8.29 (1H, s), 8.82 (1H, s).

The following compounds were synthesized analogously to Compound 87

| Comp. | | | |
|---|---|---|---|
| No | Structure | Name | ¹H NMR (400 MHz) |
| 88 | 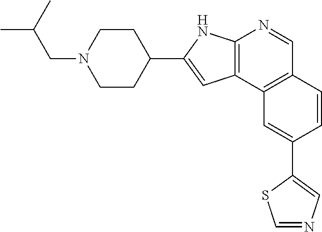 [SMILES: CC(C)CN1CCC(c2cc3c(ncc4ccc(-c5cncs5)cc43)[nH]2)CC1] | 5-(2-(1- isobutylpiperidin-4- yl)-3H-pyrrolo[2,3- c]isoquinolin-8- yl)thiazole | DMSO-d₆; δ 0.89 (6H, d, J = 6.8 Hz), 1.70-1.89 (3H, m), 1.99-2.14 (6H, m), 2.74-2.86 (1H, m), 2.93-3.00 (2H, m), 6.93 (1H, d, J = 2.0 Hz), 7.80 (1H, dd, J = 8.4, 1.6 Hz), 8.12- 8.19 (1H, m), 8.52 (1H, s), 8.59 (1H, s), 8.81 (1H, s), 9.19 (1H, s), 11.89 (1H, brs). |
| 89 | 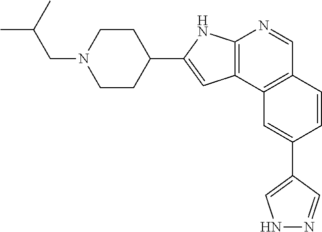 [SMILES: CC(C)CN1CCC(c2cc3c(ncc4ccc(-c5cn[nH]c5)cc43)[nH]2)CC1] | 2-(1- isobutylpiperidin-4- yl)-8-(1H-pyrazol- 4-yl)-3H- pyrrolo[2,3- c]isoquinoline | DMSO-d₆; δ 0.92 (6H, d, J = 6.8 Hz), 1.76-1.93 (3H, m), 2.05-2.25 (6H, m), 2.77-2.90 (1H, m), 2.99-3.10 (2H, m), 6.84 (1H, d, J = 2.0 Hz), 7.75 (1H, dd, J = 8.4, 1.2 Hz), 8.05 (1H, d, J = 8.4 Hz), 8.32 (2H, s), 8.44 (1H, s), 8.74 (1H, s), 11.78 (1H, brs). |

### Intermediates of Formula (II)

Schemes below illustrate the preparation of intermediates.

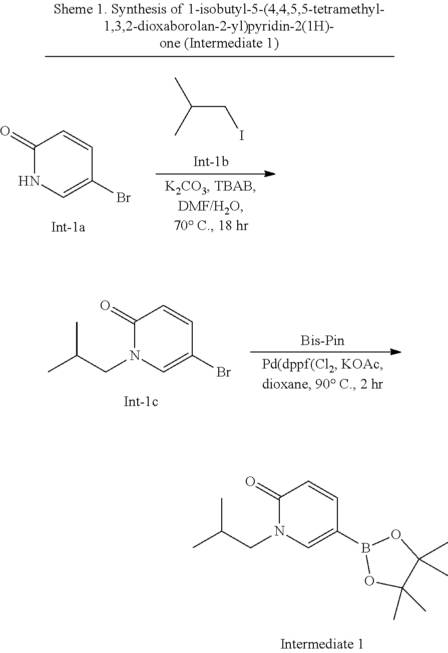

### Step 1. Synthesis of 5-bromo-1-isobutylpyridin-2(1H)-one

A mixture of 5-bromopyridin-2(1H)-one (1.00 g, 5.75 mmol), Int-1b (1.27 g, 6.90 mmol), TBAB (185 mg, 0.574 mmol) and K₂CO₃ (3.97 g, 28.7 mmol) in DMF (20 mL) and H₂O (2 mL) was stirred at 70° C. for 18 hours. The reaction mixture was diluted with H₂O (30 mL) and extracted with EtOAc (40 mL×3). The combined organic layer was washed with brine (50 mL), dried over anhydrous Na₂SO₄, filtered and concentrated. The residue was purified by flash silica gel column (PE/EtOAc=2/1) to afford compound 3 (580 mg, yield: 44%) as yellow oil.

¹H NMR (400 MHz, CDCl₃) δ 0.96 (6H, d, J=6.4 Hz), 2.08-2.25 (1H, m), 3.72 (2H, d, J=7.6 Hz), 6.32-6.60 (1H, m), 7.31-7.38 (2H, m).

### Step 2. Synthesis of 1-isobutyl-5-(4,4,5,5-tetramethyl-1, 3,2-dioxaborolan-2-yl)pyridin-2(1H)-one

A mixture of 5-bromo-1-isobutylpyridin-2(1H)-one (530 mg, 2.30 mmol), Bis-Pin (701 mg, 2.76 mmol), Pd(dppf)Cl₂ (168 mg, 0.230 mmol) and KOAc (678 mg, 6.91 mmol) in 1, 4-dioxane (10 mL) was degassed and purged with N₂ for 3 times, then the mixture was stirred at 90° C. for 2 hours under N₂ atmosphere. The reaction mixture was concentrated and the residue was purified by flash silica gel column (PE/EtOAc=1/1) to afford 1-isobutyl-5-(4,4,5,5-tetramethyl-1,3,2-dioxaborolan-2-yl)pyridin-2(1H)-one (680 mg, yield: 98%) as yellow oil.

¹H NMR (400 MHz, CDCl₃) δ 0.95 (6H, d, J=6.8 Hz), 1.30-1.35 (13H, m), 3.77 (2H, d, J=7.2 Hz), 4.08-4.18 (1H, m), 6.60 (1H, d, J=8.8 Hz), 7.62 (1H, d, J=8.8 Hz), 7.69 (1H, s).

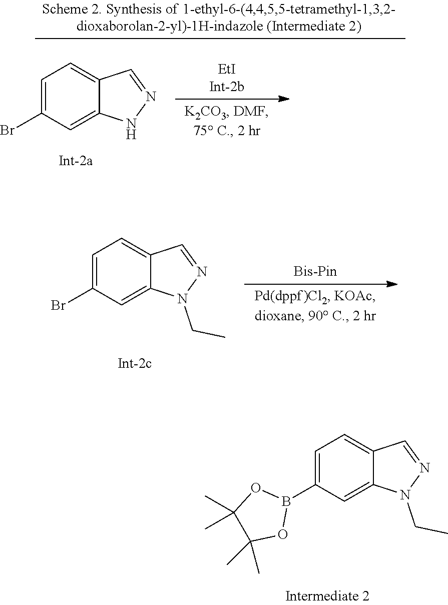 [SMILES: Brc1ccc2cn[nH]c2c1 ; CCn1ncc2ccc(Br)cc21 ; CCn1ncc2ccc(B3OC(C)(C)C(C)(C)O3)cc21]

### Step 1. Synthesis of 6-bromo-1-ethyl-1H-indazole

A mixture of 6-bromo-1H-indazole (2.00 g, 10.2 mmol), EtI (3.17 g, 20.3 mmol) and K₂CO₃ (4.21 g, 30.4 mmol) in DMF (20 mL) was stirred at 75° C. for 2 hours. The reaction mixture was diluted with H₂O (50 mL) and extracted with EtOAc (50 mL×3). The combined organic layer was washed with brine (50 mL), dried over anhydrous Na₂SO₄, filtered and concentrated. The residue was purified by flash silica gel column (PE/EtOAc=2/1) to afford 6-bromo-1-ethyl-1H-indazole (1.50 g, yield: 66%) as colorless oil.

¹H NMR (400 MHz, CDCl₃) δ 1.52 (3H, t, J=7.6 Hz), 4.40 (2H, q, J=7.6 Hz), 7.25 (1H, dd, J=8.4, 1.2 Hz), 7.55-7.65 (2H, m), 7.96 (1H, s).

### Step 2. Synthesis of I-ethyl-6-(4,4,5,5-tetramethyl-1, 3,2-dioxaborolan-2-yl)-1H-indazole

A mixture of 6-bromo-1-ethyl-1H-indazole (900 mg, 4.00 mmol), Bis-Pin (1.22 g, 4.80 mmol), Pd(dppf)Cl₂ (292 mg, 0.399 mmol) and KOAc (1.18 g, 12.00 mmol) in 1, 4-dioxane (20 mL) was degassed and purged with N₂ for 3 times, then the mixture was stirred at 90° C. for 2 hours under N₂ atmosphere. The reaction mixture was concentrated and the residue was purified by flash silica gel column (PE/EtOAc=2/1) to afford 1-ethyl-6-(4,4,5,5-tetramethyl-1,3,2-dioxaborolan-2-yl)-1H-indazole (1.50 g, crude) as yellow oil.

¹H NMR (400 MHz, CDCl₃) δ 1.40 (12H, s), 1.54 (3H, t, J=7.2 Hz), 4.50 (2H, q, J=7.2 Hz), 7.56 (1H, d, J=8.0 Hz), 7.73 (1H, d, J=8.0 Hz), 7.94 (1H, s), 8.01 (1H, s).

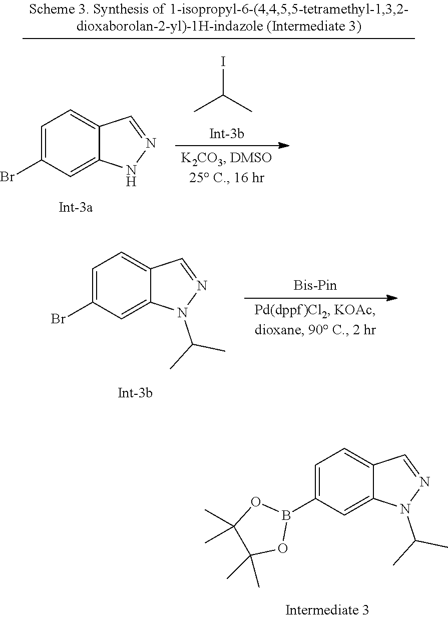

### Step 1. Synthesis of 6-bromo-1-isopropyl-1H-indazole

To a solution of 6-bromo-1H-indazole (200 mg, 1.02 mmol) in DMSO (5 mL) was added K₂CO₃ (281 mg, 2.03 mmol) and 2-iodopropane (207 mg, 1.22 mmol). The mixture was stirred at 25° C. for 16 hours. The reaction mixture was diluted with H₂O (25 mL) and extracted with EtOAc (30 mL×3). The combined organic layers were washed with brine (40 mL), dried over anhydrous Na₂SO₄, filtered and concentrated. The residue was purified by silica gel column (0% to 14% EtOAc in PE) to give 6-bromo-1-isopropyl-1H-indazole (150 mg, yield: 62%) as yellow oil.

¹H NMR (400 MHz, CDCl₃) δ 1.59 (6H, d, J=6.8 Hz), 4.69-4.87 (1H, m), 7.24 (1H, dd, J=8.8, 1.6 Hz), 7.59 (1H, d, J=8.8 Hz), 7.63 (1H, s), 7.98 (1H, s).

### Step 2. Synthesis of 1-isopropyl-6-(4,4,5,5-tetramethyl-1,3,2-dioxaborolan-2-yl)-1H-indazole

A mixture of 6-bromo-1-isopropyl-1H-indazole (150 mg, 0.627 mmol), Bis-Pin (239 mg, 0.941 mmol), Pd(dppf)Cl₂ (46 mg, 0.062 mmol) and KOAc (185 mg, 1.88 mmol) in dioxane (5 mL) was degassed and purged with N₂ for 3 times, then the mixture was stirred at 90° C. for 2 hours under N₂ atmosphere. The reaction mixture was concentrated and the residue was purified by silica gel column (0% to 5% EtOAc in PE) to give 1-isopropyl-6-(4,4,5,5-tetramethyl-1,3,2-dioxaborolan-2-yl)-1H-indazole (150 mg, yield: 83%) as a white solid.

¹H NMR (400 MHz, CDCl₃) δ 1.41 (12H, s), 1.62 (6H, d, J=6.8 Hz), 4.93-5.04 (1H, m), 7.57 (1H, d, J=8.4 Hz), 7.74 (1H, d, J=8.0 Hz), 7.98 (1H, s), 8.04 (1H, s).

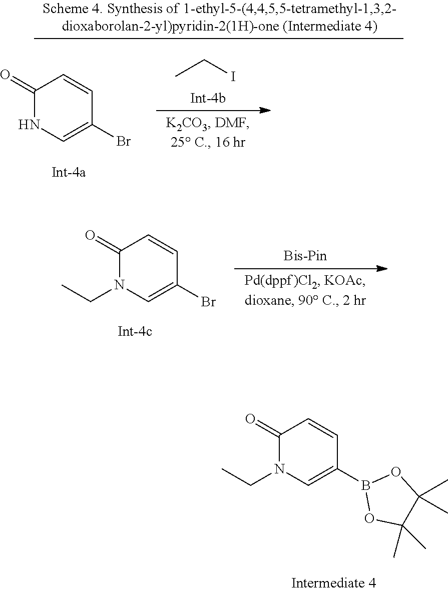

### Step 1. Synthesis of 5-bromo-1-ethylpyridin-2(1H)-one

A mixture of 5-bromopyridin-2(1H)-one (200 mg, 1.15 mmol), iodoethane (215 mg, 1.38 mmol) and K₂CO₃ (477 mg, 3.45 mmol) in DMF (5 mL) was stirred at 25° C. for 16 hours under N₂ atmosphere. The reaction mixture was diluted with H₂O (25 mL) and extracted with EtOAc (30 mL×3). The combined organic layers were washed with brine (40 mL), dried over anhydrous Na₂SO₄, filtered and concentrated. The residue was purified by silica gel column (0% to 35% EtOAc in PE) to give 5-bromo-1-ethylpyridin-2(1H)-one (120 mg, yield: 52%) as a yellow solid.

¹H NMR (400 MHz, DMSO-d₆) δ 1.20 (3H, t, J=7.2 Hz), 3.88 (2H, q, J=7.2 Hz), 6.36 (1H, d, J=9.6 Hz), 7.51 (1H, dd, J=9.6, 2.8 Hz), 8.04 (1H, d, J=2.8 Hz).

### Step 2. Synthesis of I-ethyl-5-(4,4,5,5-tetramethyl-1, 3,2-dioxaborolan-2-yl)pyridin-2(1H)-one (Intermediate 4)

A mixture of 5-bromo-1-ethylpyridin-2(1H)-one (120 mg, 0.594 mmol), Bis-Pin (302 mg, 1.19 mmol), Pd(dppf)Cl₂ (87 mg, 0.12 mmol) and KOAc (175 mg, 1.78 mmol) in dioxane (5 mL) was degassed and purged with N₂ for 3 times, then the mixture was stirred at 90° C. for 2 hours under N₂ atmosphere. The reaction mixture was concentrated and the residue was purified by silica gel column (0% to 35% EtOAc in PE) to give 1-ethyl-5-(4,4,5,5-tetramethyl-1,3,2-dioxaborolan-2-yl)pyridin-2(1H)-one (110 mg, yield: 74%) as yellow gum.

¹H NMR (400 MHz, DMSO-d₆) δ 1.19 (3H, t, J=7.2 Hz), 1.27 (12H, s), 3.93-3.97 (2H, m), 6.35 (1H, d, J=8.8 Hz), 7.48 (1H, dd, J=9.2, 2.0 Hz), 7.98 (1H, d, J=1.6 Hz).

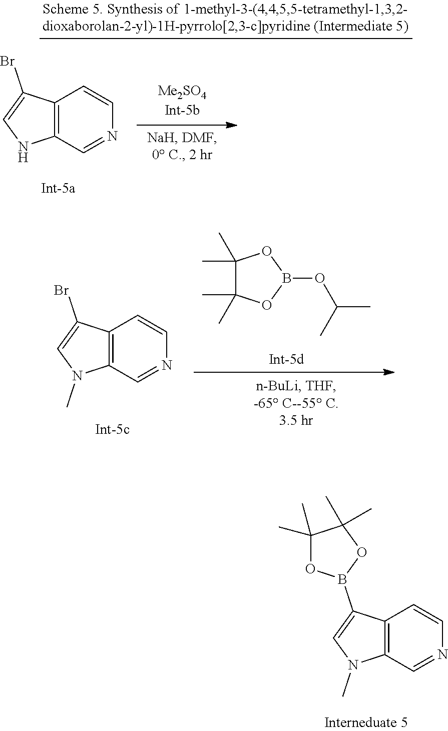 [SMILES: Brc1c[nH]c2cnccc12 ; Cn1cc(Br)c2ccncc21 ; CC(C)OB1OC(C)(C)C(C)(C)O1 ; Cn1cc(B2OC(C)(C)C(C)(C)O2)c2ccncc21]

### Step 1. Synthesis of 3-bromo-1-methyl-1H-pyrrolo[2, 3-c]pyridine

To a solution of 3-bromo-1H-pyrrolo[2,3-c]pyridine (2.50 g, 12.7 mmol) in DMF (20 mL) was added NaH (609 mg, 15.2 mmol, 60% dispersion in mineral oil) at 0° C. After stirring at 0° C. for 0.5 hour, a solution of Me₂SO₄ (1.92 g, 15.2 mmol) in DMF (5 mL) was added to the reaction mixture and the resulting reaction mixture was stirred at 0° C. for another 1.5 hours under N₂ atmosphere. The reaction mixture was quenched with saturated aqueous NaHCO₃ (20 mL) at 0° C., then diluted with water (30 mL) and extracted with DCM (50 mL×3). The combined organic layer was dried over anhydrous Na₂SO₄, filtered and concentrated. The residue was purified by flash silica gel column (MeOH/DCM=1/20) to afford 3-bromo-1-methyl-1H-pyrrolo[2,3-c]pyridine (1.73 g, yield: 62%) as a yellow solid.

¹H NMR (400 MHz, DMSO-d₆) δ 3.92 (3H, s), 7.40 (1H, d, J=5.6 Hz), 7.79 (1H, s), 8.24 (1H, d, J=5.6 Hz), 8.90 (1H, s)

### Step 2. Synthesis of 1-methyl-3-(4,4,5,5-tetramethyl-1, 3,2-dioxaborolan-2-yl)-1H-pyrrolo[2, 3-c]pyridine

To a solution of 3-bromo-1-methyl-1H-pyrrolo[2,3-c]pyridine (280 mg, 1.33 mmol) in THF (7 mL) was added n-BuLi (0.95 mL, 2.39 mmol, 2.5M in hexane) dropwise at −65° C. and stirred for 0.5 hour under N₂ atmosphere. A solution of Int-5d (370 mg, 1.99 mmol) in THF (1 mL) was added dropwise to the reaction mixture at −65° C. After the completion of the addition, the mixture was stirred at −55° C. for another 3 hours under N₂ atmosphere. The reaction mixture was quenched with saturated aqueous NH₄Cl (10 mL) and diluted with water (10 mL), then extracted with DCM (20 mL×3). The combined organic layer was dried over anhydrous Na₂SO₄, filtered and concentrated. The residue was purified by flash silica gel column (EtOAC/PE=1/1) to afford 1-methyl-3-(4,4,5,5-tetramethyl-1,3,2-dioxaborolan-2-yl)-1H-pyrrolo[2,3-c]pyridine (340 mg, yield: 60%) as a brown solid.

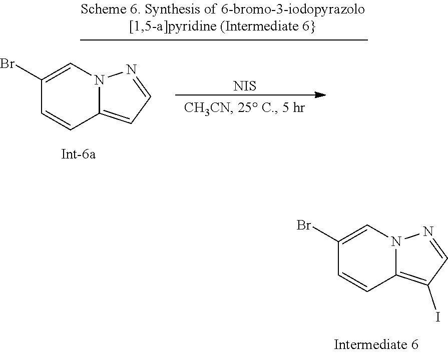 [SMILES: Brc1ccc2ccnn2c1 ; "NIS" ; Brc1ccc2c(I)cnn2c1]

A mixture of 6-bromopyrazolo[1,5-a]pyridine (1.00 g, 5.08 mmol), NIS (1.43 g, 6.34 mmol) in CH₃CN (10 mL) was stirred at 25° C. for 5 hours under N₂ atmosphere. The reaction mixture was concentrated and the crude product was triturated with CH₃CN (10 mL) to give 6-bromo-3-iodopyrazolo[1,5-a]pyridine (1.50 g, yield: 92%) as a gray solid.

¹H NMR (400 MHz, DMSO-d₆) δ 7.30-7.64 (2H, m), 7.94-8.25 (1H, m), 9.17 (1H, s).

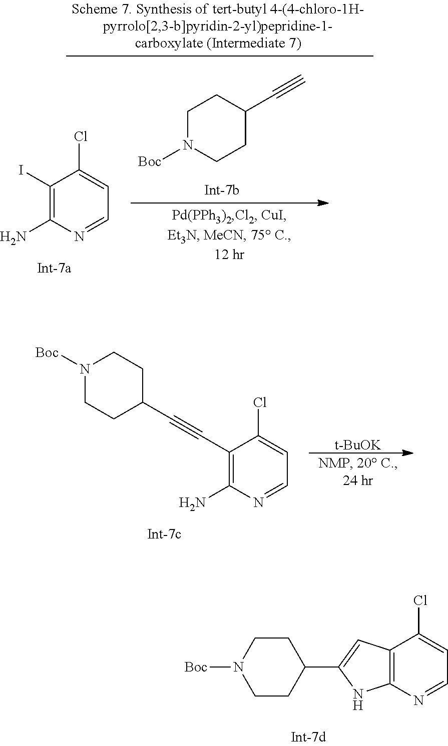 [SMILES: "t-BuOK" ; Nc1nccc(Cl)c1I ; C#CC1CCN(C(=O)OC(C)(C)C)CC1 ; CC(C)(C)OC(=O)N1CCC(C#Cc2c(Cl)ccnc2N)CC1 ; CC(C)(C)OC(=O)N1CCC(c2cc3c(Cl)ccnc3[nH]2)CC1]

### Step 1. Synthesis of tert-butyl 4-((2-amino-4-chloropyridin-3-yl)ethynyl)piperidine-1-carboxylate

A mixture of 4-chloro-3-iodopyridin-2-amine (500 mg, 1.96 mmol), tert-butyl 4-ethynylpiperidine-1-carboxylate (411 mg, 1.96 mmol), CuI (19 mg, 0.098 mmol), Pd(PPh₃)₂Cl₂ (41 mg, 0.059 mmol) and Et₃N (1.39 g, 13.8 mmol) in MeCN (5 mL) was degassed and purged with N₂ for 3 times, and then the mixture was stirred at 75° C. for 12 hours under N₂ atmosphere. The reaction mixture was concentrated and the residue was diluted with H₂O (50 mL) and extracted with EtOAc (60 mL×3). The combined organic layers were washed with brine (100 mL), dried over anhydrous Na₂SO₄, filtered and concentrated. The residue was purified by silica gel column (0% to 15% EtOAc in PE) to give of tert-butyl 4-((2-amino-4-chloropyridin-3-yl)ethynyl)piperidine-1-carboxylate (500 mg, yield: 76%) as a yellow solid.

¹H NMR (400 MHz, DMSO-d₆) δ 1.43 (9H, s), 1.53-1.67 (2H, m), 1.77-1.89 (2H, m), 2.89-3.03 (1H, m), 3.11-3.27 (2H, m), 3.54-3.70 (2H, m), 6.43 (2H, brs), 6.68 (1H, d, J=5.2 Hz), 7.85 (1H, d, J=5.6 Hz).

### Step 2. Synthesis of tert-butyl 4-(4-chloro-1H-pyrrolo[2,3-b]pyridin-2-yl)piperidine-1-carboxylate

To a solution of tert-butyl 4-((2-amino-4-chloropyridin-3-yl)ethynyl)piperidine-1-carboxylate (500 mg, 1.49 mmol) in NMP (5 mL) was added t-BuOK (368 mg, 3.28 mmol). The mixture was stirred at 20° C. for 24 hours. The reaction mixture was diluted with saturated aqueous NH₄Cl (50 mL) and filtered. The solid was washed with water (10 mL×2) and dried to give tert-butyl 4-(4-chloro-1H-pyrrolo[2,3-b]pyridin-2-yl)piperidine-1-carboxylate (500 mg, yield: 100%) as a yellow solid.

¹H NMR (400 MHz, DMSO-d₆) δ 1.42 (9H, s), 1.49-1.68 (2H, m), 1.93-2.05 (2H, m), 2.76-3.02 (3H, m), 3.93-4.16 (2H, m), 6.24 (1H, s), 7.13 (1H, d, J=5.2 Hz), 8.08 (1H, d, J=5.2 Hz), 11.96 (1H, brs).

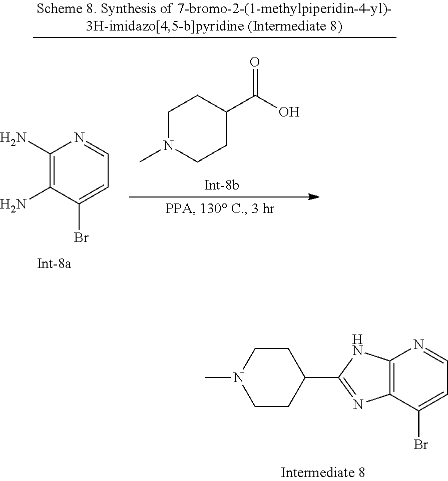

To a solution of 4-bromopyridine-2,3-diamine (350 mg, 1.86 mmol) in PPA (6 mL) was added 1-methylpiperidine-4-carboxylic acid (533 mg, 3.72 mmol). The mixture was stirred at 130° C. for 3 hours. The reaction mixture was diluted with water (50 mL) and basified with 2N aqueous NaOH to pH=10, then extracted with DCM (100 mL×3). The combined organic layers were dried over anhydrous Na₂SO₄, filtered and concentrated to give 7-bromo-2-(1-methylpiperidin-4-yl)-3H-imidazo[4,5-b]pyridine (490 mg, yield: 89%) as a gray solid.

¹H NMR (400 MHz, DMSO-d₆) δ 1.81-1.91 (2H, m), 1.96-2.04 (4H, m), 2.20 (3H, s), 2.80-2.90 (3H, m), 7.45 (1H, d, J=5.2 Hz), 8.10 (1H, d, J=4.8 Hz), 13.12 (1H, brs).

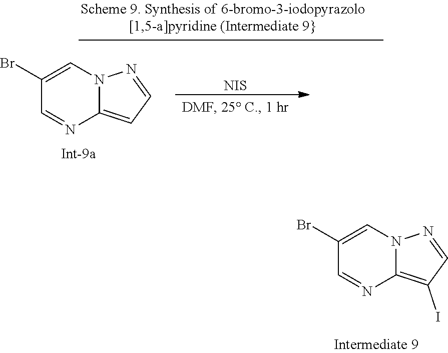 [SMILES: Brc1cnc2ccnn2c1 ; "NIS" ; Brc1cnc2c(I)cnn2c1]

A solution of 6-bromopyrazolo[1,5-a]pyrimidine (500 mg, 2.52 mmol) and NIS (624 mg, 2.78 mmol) in DMF (5 mL) was stirred at 25° C. for 1 hour. The reaction mixture was poured into H₂O (25 mL) and the precipitate was filtered and dried to afford 6-bromo-3-iodopyrazolo[1,5-a]pyrimidine (600 mg, yield: 73%) as a yellow solid.

¹H NMR (400 MHz, DMSO-d₆) δ 8.33 (1H, s), 8.67 (1H, d, J=2.0 Hz), 9.64 (1H, d, J=2.0 Hz).

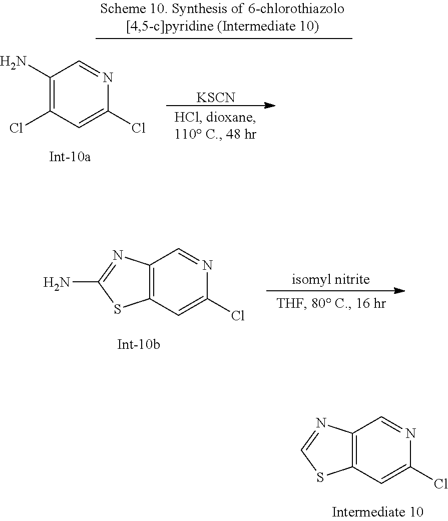 [SMILES: Nc1cnc(Cl)cc1Cl ; "KSCN" ; Nc1nc2cnc(Cl)cc2s1 ; Clc1cc2scnc2cn1]

### Step 1. Synthesis of 6-chlorothiazolo[4,5-c]pyridin-2-amine

To a solution of 4,6-dichloropyridin-3-amine (750 mg, 4.60 mmol) in dioxane (15 mL) was added KSCN (1.34 g, 13.8 mmol) and conc. HCl (0.05 mL). The mixture was stirred at 110° C. for 48 hours. The reaction mixture was concentrated and the residue was purified by silica gel column (0% to 50% EtOAc in PE) to give compound 2 (400 mg, yield: 47%) as a yellow solid.

¹H NMR (400 MHz, DMSO-d₆) δ 7.89 (1H, s), 7.95 (2H, brs), 8.33 (1H, s).

### Step 2. Synthesis of 6-chlorothiazolo[4,5-c]pyridine

To a solution of compound 2 (150 mg, 0.808 mmol) in THF (5 mL) was added isopentyl nitrite (189 mg, 1.62 mmol). The mixture was stirred at 80° C. for 16 hours. The reaction mixture was concentrated and the residue was purified by silica gel column (0% to 18% EtOAc in PE) to give 6-chlorothiazolo[4,5-c]pyridine (80 mg, yield: 58%) as a yellow solid.

¹H NMR (400 MHz, DMSO-d₆) δ 8.43 (1H, s), 9.20 (1H, s), 9.54 (1H, s).

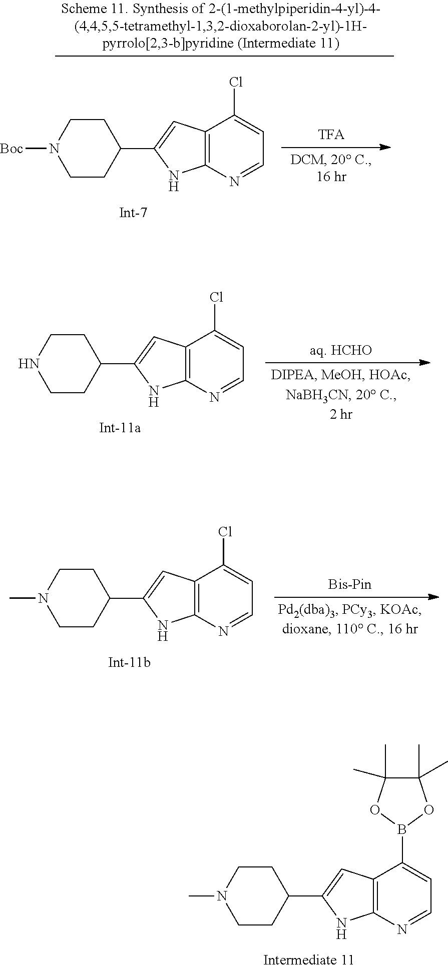

### Step 1. Synthesis of 4-chloro-2-(piperidin-4-yl)-1H-pyrrolo[2,3-b]pyridine

To a solution of Intermediate 7 (400 mg, 1.19 mmol) in DCM (4 mL) was added TFA (1 mL). The mixture was stirred at 20° C. for 16 hours. The reaction mixture was concentrated to give 4-chloro-2-(piperidin-4-yl)-1H-pyrrolo[2,3-b]pyridine (280 mg, crude, TFA salt) as yellow gum.

### Step 2. Synthesis of 4-chloro-2-(1-methylpiperidin-4-yl)-1H-pyrrolo[2,3-b]pyridine

To a solution of 4-chloro-2-(piperidin-4-yl)-1H-pyrrolo[2,3-b]pyridine (280 mg, 1.19 mmol, TFA salt) in MeOH (5 mL) was added DIPEA (154 mg, 1.19 mmol). The mixture was stirred at 20° C. for 0.5 hour. Then HOAc (71 mg, 1.2 mmol) was added to the mixture to adjust pH=5, 37% aqueous HCHO (482 mg, 5.94 mmol) was added and stirred at 20° C. for 0.5 hour. NaBH₃CN (224 mg, 3.56 mmol) was added and the mixture was stirred at 20° C. for another 1 hour. The reaction mixture was quenched by addition saturated aqueous.NaHCO₃ (30 mL) and extracted with DCM (30 mL×3). The combined organic layers were washed with brine (30 mL), dried over anhydrous Na₂SO₄, filtered and concentrated to give 4-chloro-2-(1-methylpiperidin-4-yl)-1H-pyrrolo[2,3-b]pyridine (290 mg, yield: 98%) as a yellow solid.

### Step 3. Synthesis of 2-(1-methylpiperidin-4-yl)-4-(4,4,5,5-tetramethyl-1, 3,2-dioxaborolan-2-yl)-1H-pyrrolo[2,3-b]pyridine

A mixture of 4-chloro-2-(1-methylpiperidin-4-yl)-1H-pyrrolo[2,3-b]pyridine (50 mg, 0.200 mmol), Bis-Pin (66 mg, 0.26 mmol), KOAc (39 mg, 0.40 mmol), PCy₃ (11 mg, 0.040 mmol) and Pd₂(dba)₃ (18 mg, 0.020 mmol) in dioxane (2 mL) was degassed and purged with N₂ for 3 times, and then the mixture was stirred at 110° C. for 16 hours under N₂ atmosphere. The reaction mixture was diluted with dioxane (20 mL) and filtered. The filtrate was concentrated to give 2-(1-methylpiperidin-4-yl)-4-(4,4,5,5-tetramethyl-1,3,2-dioxaborolan-2-yl)-1H-pyrrolo[2,3-b]pyridine (60 mg, crude) as yellow gum.

Compounds of Formula (II)

### Example 1

### 1-methyl-5-(3-(1-methyl-1H-indazol-6-yl)pyrazolo[1,5-a]pyridin-6-yl)pyridin-2(1H)-one

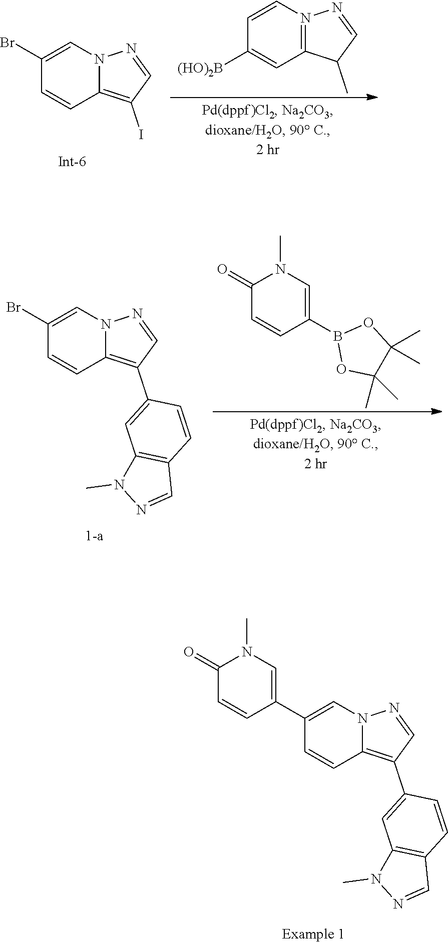

### Step 1. Synthesis of 6-(6-bromopyrazolo[1,5-a]pyridin-3-yl)-1-methyl-1H-indazole

A mixture of Intermediate 6 (300 mg, 0.929 mmol), (1-methyl-1H-indazol-6-yl)boronic acid (163 mg, 0.929 mmol), Pd(dppf)Cl₂ (68 mg, 0.093 mmol) and Na₂CO₃ (295 mg, 2.79 mmol) in dioxane (2 mL) and H₂O (0.5 mL) was degassed and purged with N₂ for 3 times. Then the mixture was stirred at 90° C. for 2 hours under N₂ atmosphere. The reaction mixture was filtered and the filtrate was concentrated. The residue was purified by flash silica gel chromatography (ISCO®; 12 g SepaFlash® Silica Flash Column, Eluent of 0˜20% Ethyl acetate/Petroleum ether gradient @35 mL/min), then further triturated with MeOH (5 mL) to give 6-(6-bromopyrazolo[1,5-a]pyridin-3-yl)-1-methyl-TH-indazole (120 mg, yield: 39%) as a yellow solid.

¹H NMR (400 MHz, DMSO-d₆) 3 4.11 (3H, s), 7.47 (1H, dd, J=2.8, 1.6 Hz), 7.48-7.50 (1H, m), 7.83 (1H, d, J=8.4 Hz), 7.88 (1H, s), 8.05 (1H, s), 8.11 (1H, d, J=9.2 Hz), 8.50 (1H, s), 9.17 (1H, d, J=0.8 Hz).

### Step 2. Synthesis of 1-methyl-5-(3-(1-methyl-1H-indazol-6-yl)pyrazolo[1,5-a]pyridin-6-yl)pyridin-2(JH)-one

A mixture of 6-(6-bromopyrazolo[1,5-a]pyridin-3-yl)-1-methyl-1H-indazole (100 mg, 0.306 mmol), 1-methyl-5-(4,4,5,5-tetramethyl-1,3,2-dioxaborolan-2-yl)pyridin-2(1H)-one (108 mg, 0.458 mmol), Pd(dppf)Cl₂ (22 mg, 0.031 mmol) and Na₂CO₃ (97 mg, 0.92 mmol) in dioxane (2 mL) and H₂O (0.5 mL) was degassed and purged with N₂ for 3 times. Then the mixture was stirred at 90° C. for 2 hours under N₂ atmosphere. The reaction mixture was filtered and the filtrate was concentrated. The residue was purified by flash silica gel chromatography (ISCO®; 12 g SepaFlash® Silica Flash Column, Eluent of 0˜4% MeOH/DCM@30 mL/min), then further triturated with MeOH (2 mL) to give 1-methyl-5-(3-(1-methyl-TH-indazol-6-yl)pyrazolo[1,5-a]pyridin-6-yl)pyridin-2(1H)-one (80.29 mg, yield: 73%) as a yellow solid.

¹H NMR (400 MHz, DMSO-d₆) δ 3.54 (3H, s), 4.12 (3H, s), 6.54 (1H, d, J=9.2 Hz), 7.52 (1H, d, J=8.4 Hz), 7.65 (1H, d, J=9.6 Hz), 7.84 (1H, d, J=8.4 Hz), 7.89 (1H, s), 7.99 (1H, dd, J=9.6, 2.4 Hz), 8.05 (1H, s), 8.20 (1H, d, J=9.2 Hz), 8.32 (1H, d, J=2.4 Hz), 8.50 (1H, s), 9.06 (1H, s).

The following compounds were synthesized analogously to Example 1

| Example | | | |
|---|---|---|---|
| No. | Structure | Name | ¹H NMR (400 MHz) |
| 2 | 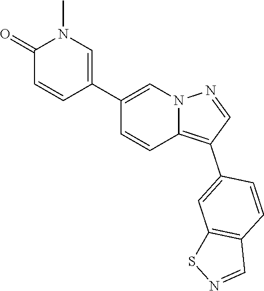 [SMILES: Cn1cc(-c2ccc3c(-c4ccc5cnsc5c4)cnn3c2)ccc1=O] | 5-(3- (benzo[d]isothiazol- 6-yl)pyrazolo[1,5- a]pyridin-6-yl)-1- methylpyridin- 2(1H)-one | DMSO-d₆; δ 3.55 (3H, s), 6.54 (1H, d, J = 9.6 Hz), 7.71 (1H, dd, J = 9.2, 1.6 Hz), 7.92 (1H, dd, J = 8.4, 1.6 Hz), 8.00 (1H, d, J = 9.6 Hz), 8.23-8.30 (2H, m), 8.34 (1H, d, J = 2.4 Hz), 8.55-8.68 (2H, m), 9.05- 9.10 (2H, m). |
| 3 | 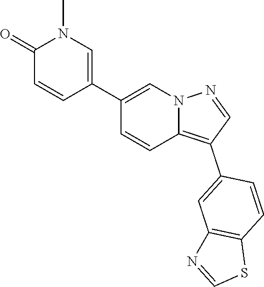 [SMILES: Cn1cc(-c2ccc3c(-c4ccc5scnc5c4)cnn3c2)ccc1=O] | 5-(3- (benzo[d]thiazol-5- yl)pyrazolo[1,5- a]pyridin-6-yl)-1- methylpyridin- 2(1H)-one | DMSO-d₆; δ 3.54 (3H, s), 6.53 (1H, d, J = 9.6 Hz), 7.66 (1H, d, J = 8.8 Hz), 7.88 (1H, dd, J = 8.4, 1.6 Hz), 7.99 (1H, dd, J = 9.6, 2.8Hz), 8.14 (1H, d, J = 9.6 Hz), 8.26 (1H, d, J = 8.4 Hz), 8.33 (1H, d, J = 2.4 Hz), 8.40 (1H, d, J = 1.6 Hz), 8.55 (1H, s), 9.07 (1H, s), 9.45 (1H, s). |
| 8 | 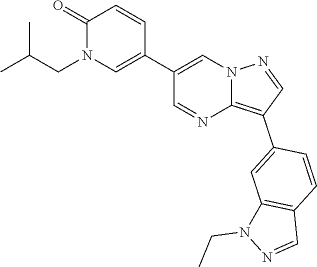 [SMILES: CCn1ncc2ccc(-c3cnn4cc(-c5ccc(=O)n(CC(C)C)c5)cnc34)cc21] | 5-(3-(1-ethyl-1H- indazol-6- yl)pyrazolo[1,5- a]pyrimidin-6-yl)- 1-isobutylpyridin- 2(1H)-one | DMSO-d₆; δ 0.91 (6H, d, J = 6.4 Hz), 1.46 (3H, t, J = 7.2 Hz), 2.10-2.26 (1H, m), 3.80 (2H, d, J = 7.2 Hz), 4.47 (2H, q, J = 7.2 Hz), 6.56 (1H, d, J = 9.6 Hz), 7.81 (1H, d, J = 8.4 Hz), 7.95-8.07 (3H, m), 8.35 (1H, s), 8.40 (1H, s), 8.89 (1H, s), 9.04 (1H, d, J = 2.4 Hz), 9.44 (1H, s). |
| 9 | 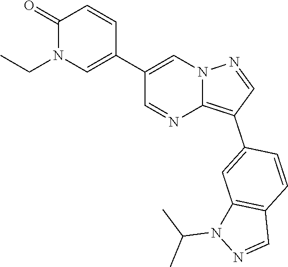 [SMILES: CCn1cc(-c2cnc3c(-c4ccc5cnn(C(C)C)c5c4)cnn3c2)ccc1=O] | 1-ethyl-5-(3-(1- isopropyl-1H- indazol-6- yl)pyrazolo[1,5- a]pyrimidin-6- yl)pyridin-2(1H)- one | DMSO-d₆; δ 1.32 (3H, t, J = 7.2 Hz), 1.55 (6H, d, J = 6.4 Hz), 4.02 (2H, q, J = 7.2 Hz), 4.96-5.07 (1H, m), 6.56 (1H, d, J = 9.6 Hz), 7.82 (1H, d, J = 8.4 Hz), 7.99-8.03 (2H, m), 8.05 (1H, s), 8.40 (1H, d, J = 2.4 Hz), 8.44 (1H, s), 8.91 (1H, s), 9.07 (1H, d, J = 2.4 Hz), 9.47 (1H, d, J = 2.4 Hz). |

### Example 4

### 6-(2-(1-methylpiperidin-4-yl)-1H-pyrrolo[2,3-b]pyridin-4-yl)thiazolo[4,5-c]pyridine

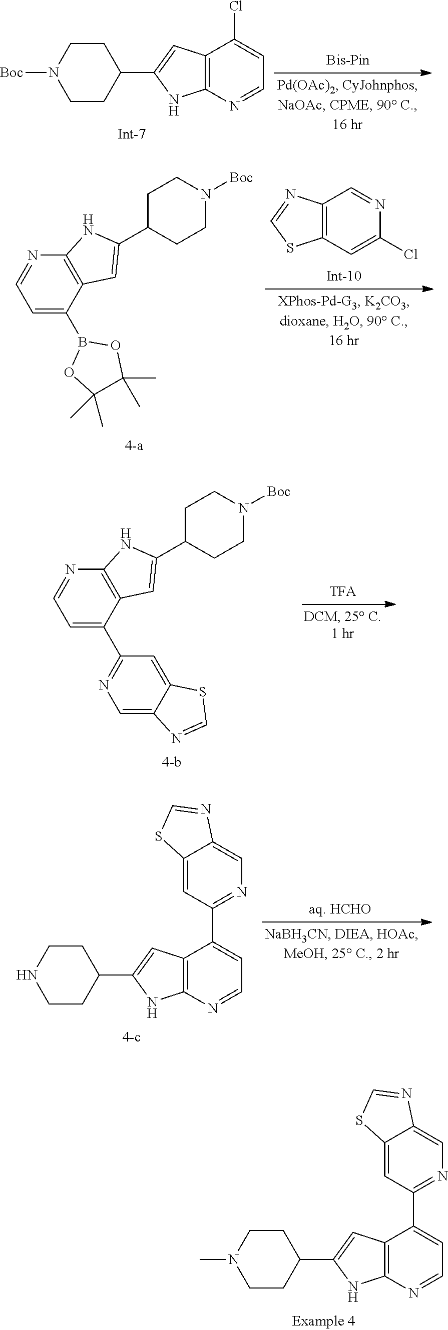 [SMILES: CC(C)(C)OC(=O)N1CCC(c2cc3c(Cl)ccnc3[nH]2)CC1 ; CC(C)(C)OC(=O)N1CCC(c2cc3c(B4OC(C)(C)C(C)(C)O4)ccnc3[nH]2)CC1 ; Clc1cc2scnc2cn1 ; CC(C)(C)OC(=O)N1CCC(c2cc3c(-c4cc5scnc5cn4)ccnc3[nH]2)CC1 ; c1cc(-c2cc3scnc3cn2)c2cc(C3CCNCC3)[nH]c2n1 ; CN1CCC(c2cc3c(-c4cc5scnc5cn4)ccnc3[nH]2)CC1]

### Step 1. Synthesis of tert-butyl 4-(4-(4,4,5,5-tetramethyl-1, 3,2-dioxaborolan-2-yl)-JH-pyrrolo[2,3-b]pyridin-2-yl)piperidine-1-carboxylate

A mixture of Intermediate 7 (400 mg, 1.19 mmol), Bis-Pin (393 mg, 1.55 mmol), NaOAc (195 mg, 2.38 mmol), CyJohnphos (42 mg, 0.12 mmol) and Pd(OAc)₂ (13 mg, 0.060 mmol) in CPME (10 mL) was degassed and purged with N₂ for 3 times, and then the mixture was stirred at 90° C. for 16 hours under N₂ atmosphere. The reaction mixture was concentrated and the residue was purified by silica gel column (0% to 60% EtOAc in PE), then the crude product was triturated with PE/EtOAc (5 mL, 1/1) to give tert-butyl 4-(4-(4,4,5,5-tetramethyl-1,3,2-dioxaborolan-2-yl)-1H-pyrrolo[2,3-b]pyridin-2-yl)piperidine-1-carboxylate (280 mg, yield: 44%) as a yellow solid.

¹H NMR (400 MHz, DMSO-d₆) δ 1.34 (12H, s), 1.43 (9H, s), 1.53-1.65 (2H, m), 1.93-2.00 (2H, m), 2.76-3.01 (3H, m), 3.98-4.17 (2H, m), 6.39 (1H, d, J=1.6 Hz), 7.23 (1H, d, J=4.8 Hz), 8.12 (1H, d, J=4.4 Hz), 11.55 (1H, brs).

### Step 2. Synthesis of tert-butyl 4-(4-(thiazolo[4,5-c]pyridin-6-yl)-1H-pyrrolo[2,3-b]pyridin-2-yl)piperidine-1-carboxylate

A mixture of Intermediate 10 (80 mg, 0.47 mmol), tert-butyl 4-(4-(4,4,5,5-tetramethyl-1,3,2-dioxaborolan-2-yl)-1H-pyrrolo[2,3-b]pyridin-2-yl)piperidine-1-carboxylate (220 mg, 0.516 mmol, XPhos-Pd-G₃ (40 mg, 0.047 mmol) and K₂CO₃ (130 mg, 0.938 mmol) in dioxane (4 mL) and H₂O (0.5 mL) was degassed and purged with N₂ for 3 times, and then the mixture was stirred at 90° C. for 16 hours under N₂ atmosphere. The reaction mixture was concentrated and the residue was purified by silica gel column (0% to 75% EtOAc in PE) to give tert-butyl 4-(4-(thiazolo[4,5-c]pyridin-6-yl)-1H-pyrrolo[2,3-b]pyridin-2-yl)piperidine-1-carboxylate (200 mg, yield: 78%) as a yellow solid.

¹H NMR (400 MHz, DMSO-d₆) δ 1.43 (9H, s), 1.57-1.71 (2H, m), 2.00-2.08 (2H, m), 2.80-3.03 (3H, m), 4.05-4.17 (2H, m), 6.88 (1H, d, J=1.6 Hz), 7.69 (1H, d, J=5.2 Hz), 8.27 (1H, d, J=5.2 Hz), 8.97 (1H, s), 9.52 (1H, s), 9.57 (1H, s), 11.76 (1H, brs).

### Step 3. Synthesis of 6-(2-(piperidin-4-yl)-1H-pyrrolo[2,3-b]pyridin-4-yl)thiazolo[4,5-c]pyridine

To a solution of tert-butyl 4-(4-(thiazolo[4,5-c]pyridin-6-yl)-1H-pyrrolo[2,3-b]pyridin-2-yl)piperidine-1-carboxylate (120 mg, 0.275 mmol) in DCM (4 mL) was added TFA (1 mL). The mixture was stirred at 25° C. for 1 hour. The reaction mixture was concentrated to give 6-(2-(piperidin-4-yl)-1H-pyrrolo[2,3-b]pyridin-4-yl)thiazolo[4,5-c]pyridine (90 mg, yield: 97%) as yellow oil.

### Step 4. Synthesis of 6-(2-(1-methylpiperidin-4-yl)-1H-pyrrolo[2,3-b]pyridin-4-yl)thiazolo[4,5-c]pyridine

To a solution of 6-(2-(piperidin-4-yl)-1H-pyrrolo[2,3-b]pyridin-4-yl)thiazolo[4,5-c]pyridine (90 mg, 0.27 mmol) in MeOH (2 mL) was added DIPEA (35 mg, 0.27 mmol), the mixture was stirred at 25° C. for 0.5 hour. Then HOAc (16 mg, 0.27 mmol) and 37% aqueous HCHO (109 mg, 1.34 mmol) were added and the mixture was stirred at 25° C. for 0.5 hour. NaBH₃CN (51 mg, 0.80 mmol) was added and the mixture was stirred at 25° C. for another 1 hour. The reaction mixture was concentrated and the residue was purified by prep-HPLC (0.225% FA as an additive), then lyophilized to afford 6-(2-(1-methylpiperidin-4-yl)-1H-pyrrolo[2,3-b]pyridin-4-yl)thiazolo[4,5-c]pyridine (48.29 mg, yield: 45%, FA salt) as a yellow solid.

¹H NMR (400 MHz, DMSO-d₆) δ 1.76-1.89 (2H, m), 2.02-2.11 (2H, m), 2.13-2.22 (2H, m), 2.30 (3H, s), 2.74-2.84 (1H, m), 2.94-3.02 (2H, m), 6.86 (1H, s), 7.67 (1H, d, J=5.2 Hz), 8.26 (1H, d, J=5.2 Hz), 8.96 (1H, s), 9.53 (1H, s), 9.57 (1H, s), 11.73 (1H, brs).

The following compounds were synthesized analogously to Example 4

| Example | | | |
|---|---|---|---|
| No. | Structure | Name | ¹H NMR (400 MHz) |
| 6 | 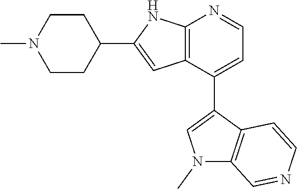 [SMILES: CN1CCC(c2cc3c(-c4cn(C)c5cnccc45)ccnc3[nH]2)CC1] | 1-methyl-3-(2-(1- methylpiperidin-4- yl)-1H-pyrrolo[2,3- b]pyridin-4-yl)-1H- pyrrolo[2,3- c]pyridine | CD₃OD; δ 2.14-2.30 (2H, m), 2.40-2.52 (2H, m), 2.95 (3H, s), 3.23-3.31 (3H, m), 3.65- 3.78 (2H, m), 4.32 (3H, s), 7.01 (1H, s), 7.88 (1H, d, J = 6.4 Hz), 8.45 (1H, d, J = 6.0 Hz), 8.47-8.51 (2H, m), 9.01 (1H, s), 9.49 (1H, s). |
| 7 | 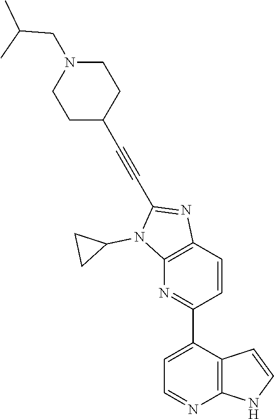 [SMILES: CC(C)CN1CCC(C#Cc2nc3ccc(-c4ccnc5[nH]ccc45)nc3n2C2CC2)CC1] | 3-cyclopropyl-2- ((1- isobutylpiperidin-4- yl)ethynyl)-5-(1H- pyrrolo[2,3- b]pyridin-4-yl)-3H- imidazo[4,5- b]pyridine | DMSO-d₆; δ 0.87 (6H, d, J = 6.4 Hz), 1.22-1.30 (3H, m), 1.32-1.38 (2H, m), 1.63-1.88 (4H, m), 1.94-2.01 (2H, m), 2.02-2.10 (2H, m), 2.15-2.25 (2H, m), 2.80-2.96 (1H, m), 3.50-3.60 (1H, m), 7.27 (1H, dd, J = 3.2, 1.6 Hz), 7.58-7.63 (1H, m), 7.67 (1H, d, J = 4.8 Hz), 8.03-8.09 (1H, m), 8.11- 8.17 (1H, m), 8.34 (1H, d, J = 5.2 Hz), 11.79 (1H, brs). |

### Example 5

### 7-(1-methyl-1H-pyrrolo[2,3-c]pyridin-3-yl)-2-(1-methylpiperidin-4-1)-3H-imidazo[4,5-b]pyridine

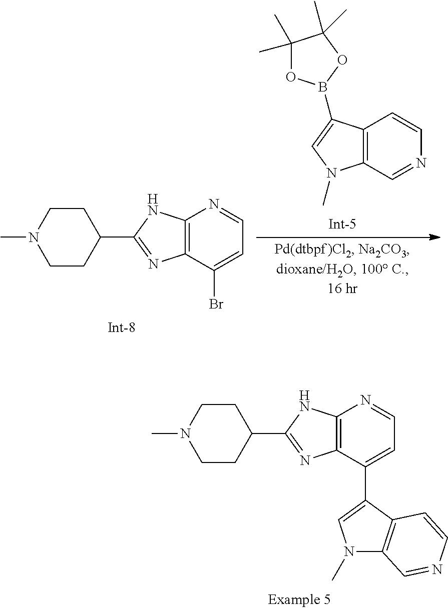 [SMILES: CN1CCC(c2nc3c(Br)ccnc3[nH]2)CC1 ; Cn1cc(B2OC(C)(C)C(C)(C)O2)c2ccncc21 ; CN1CCC(c2nc3c(-c4cn(C)c5cnccc45)ccnc3[nH]2)CC1]

A mixture of Intermediate 8 (170 mg, 0.576 mmol), Intermediate 5 (273 mg, 0.634 mmol), Pd(dtbpf)Cl₂ (56 mg, 0.086 mmol) and Na₂CO₃ (122 mg, 1.15 mmol) in dioxane (6 mL) and H₂O (1 mL) was degassed and purged with N₂ for 3 times, then the mixture was stirred at 100° C. for 16 hours under N₂ atmosphere. The reaction mixture was concentrated and the residue was diluted with water (20 mL), then extracted with EtOAc (20 mL×3). The combined organic layer was dried over anhydrous Na₂SO₄, filtered and concentrated. The residue was purified by prep-TLC (DCM/MeOH=10/1), then further purified by prep-HPLC (0.05% NH₃·H₂O as an additive) and lyophilized to afford 7-(1-methyl-1H-pyrrolo[2,3-c]pyridin-3-yl)-2-(1-methylpiperidin-4-yl)-3H-imidazo[4,5-b]pyridine (17.57 mg, yield: 9%) as a white solid.

¹H NMR (400 MHz, DMSO-d₆) δ 1.92-2.06 (6H, m), 2.22 (3H, s), 2.85-2.95 (3H, m), 4.06 (3H, s), 7.66 (1H, d, J=5.2 Hz), 8.19 (1H, d, J=5.6 Hz), 8.25 (1H, d, J=5.2 Hz), 8.30 (1H, d, J=5.6 Hz), 8.82 (1H, s), 8.97 (1H, s), 12.81 (1H, brs).

### Intermediates of Formula (III)

Schemes below illustrate the preparation of intermediates.

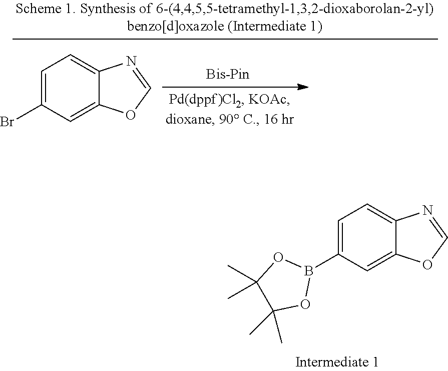 [SMILES: Brc1ccc2ncoc2c1 ; CC1(C)OB(c2ccc3ncoc3c2)OC1(C)C]

A mixture of compound 6-bromobenzo[d]oxazole (300 mg, 1.52 mmol), Bis-Pin (462 mg, 1.82 mmol), Pd(dppf)Cl₂ (111 mg, 0.152 mmol) and KOAc (446 mg, 4.55 mmol) in dioxane (2 mL) was degassed and purged with N₂ for 3 times, and then the mixture was stirred at 90° C. for 16 hours under N₂ atmosphere. The reaction mixture was filtered and the filtrate was concentrated.

The residue was purified by flash silica gel chromatography (ISCO®; 12 g SepaFlash® Silica Flash Column, Eluent of 0-10% Ethyl acetate/Petroleum ether gradient 30 mL/min) to give 6-(4,4,5,5-tetramethyl-1,3,2-dioxaborolan-2-yl)benzo[d]oxazole (300 mg, yield: 81%) as a white solid. ¹H NMR (400 MHz, CDCl₃) δ 1.39 (12H, s), 7.78-7.82 (1H, m), 7.83-7.86 (1H, m), 8.06 (1H, s), 8.16 (1H, s).

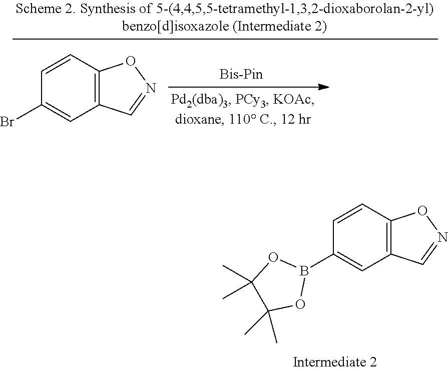 [SMILES: Brc1ccc2oncc2c1 ; CC1(C)OB(c2ccc3oncc3c2)OC1(C)C]

To a mixture of compound 5-bromobenzo[d]isoxazole (500 mg, 2.53 mmol), Bis-Pin (834 mg, 3.28 mmol), KOAc (372 mg, 3.79 mmol), Pd₂(dba)₃ (116 mg, 0.130 mmol) and PCy₃ (71 mg, 0.25 mmol) in 1, 4-dioxane (10 mL) was degassed and purged with N₂ for 3 times, and then the mixture was stirred at 110° C. for 12 hours under N₂ atmosphere. The reaction mixture was concentrated and the residue was purified by silica gel column (0% to 16% EtOAc in PE) to give 5-(4,4,5,5-tetramethyl-1,3,2-dioxaborolan-2-yl)benzo[d]isoxazole (400 mg, yield: 65%) as a yellow solid.

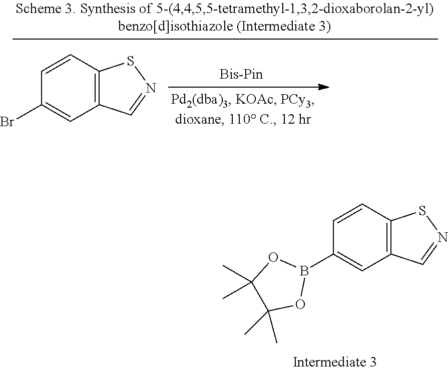 [SMILES: Brc1ccc2sncc2c1 ; CC1(C)OB(c2ccc3sncc3c2)OC1(C)C]

A mixture of compound 5-bromobenzo[d]isothiazole (150 mg, 0.701 mmol), Bis-Pin (231 mg, 0.911 mmol), KOAc (103 mg, 1.05 mmol), Pd₂(dba)₃ (32 mg, 0.035 mmol) and PCy₃ (20 mg, 0.070 mmol) in dioxane (5 mL) was degassed and purged with N₂ for 3 times, and then the mixture was stirred at 110° C. for 12 hours under N₂ atmosphere. The reaction mixture was suspended in CH₃OH (30 mL) and filtered. The filtrate was concentrated and the residue was purified by silica gel column (0% to 6% EtOAc in PE) to give 5-(4,4,5,5-tetramethyl-1,3,2-dioxaborolan-2-yl)benzo[d]isothiazole (150 mg, yield: 82%) as a yellow solid.

¹H NMR (400 MHz, CDCl₃) δ 1.41 (12H, s), 7.89-8.03 (2H, m), 8.58 (1H, s), 8.95 (1H, s).

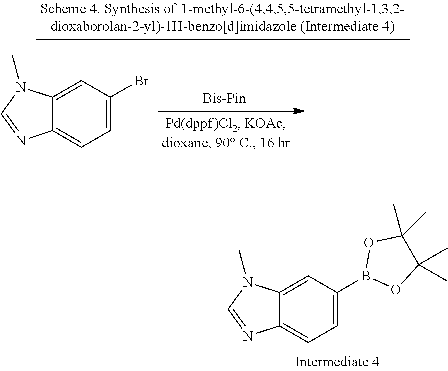 [SMILES: Cn1cnc2ccc(Br)cc21 ; Cn1cnc2ccc(B3OC(C)(C)C(C)(C)O3)cc21]

A mixture of 6-bromo-1-methyl-1H-benzo[d]imidazole (1.00 g, 4.74 mmol), Bis-Pin (1.44 g, 5.69 mmol), KOAc (1.40 g, 14.2 mmol) and Pd(dppf)Cl₂ (347 mg, 0.474 mmol) in 1, 4-dioxane (20 mL) was degassed and purged with N₂ for 3 times and then stirred at 90° C. for 16 hours under N₂ atmosphere. The reaction mixture was concentrated and the residue was purified by silica gel column (PE/EtOAc=0/1) to afford 1-methyl-6-(4,4,5,5-tetramethyl-1,3,2-dioxaborolan-2-yl)-1H-benzo[d]imidazole (880 mg, yield: 72%) as a yellow solid.

¹H NMR (400 MHz, DMSO-d₆) δ 1.32 (12H, s), 3.87 (3H, s), 7.52 (1H, d, J=8.0 Hz), 7.63 (1H, d, J=8.0 Hz), 7.87 (1H, s), 8.24 (1H, s).

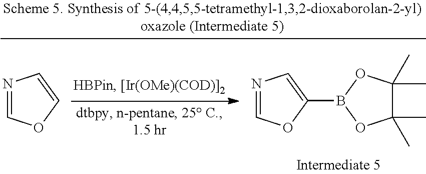

A solution of [Ir(OMe)(COD)]2 (72 mg, 0.11 mmol) and HBPin (1.85 g, 14.5 mmol) in n-pentane (7 mL) was stirred at 25° C. for 20 minutes under N₂ atmosphere, then a solution of dtbpy (59 mg, 0.22 mmol) in n-pentane (3 mL) was added dropwise to the reaction mixture at 25° C. and stirred for another 20 minutes under N₂ atmosphere. A solution of oxazole (500 mg, 7.24 mmol) in n-pentane (6 mL) was added dropwise to the reaction mixture at 25° C., and the resulting reaction mixture was stirred at 25° C. for 50 minutes. The reaction mixture was filtered and the filtrate was concentrated. The residue was purified by Combi-Flash (0 to 10% EtOAc in PE) to give 5-(4,4,5,5-tetramethyl-1,3,2-dioxaborolan-2-yl)oxazole (1.30 g, yield: 52%) as colorless oil.

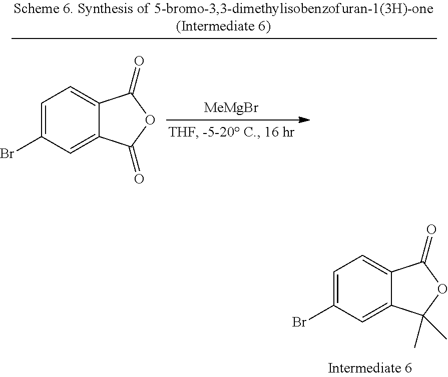

To a solution of 5-bromoisobenzofuran-1,3-dione (1.00 g, 4.41 mmol) in THF (10 mL) was added MeMgBr (3.2 mL, 9.69 mmol, 3M in THF) dropwise at −5° C. Then the mixture was stirred at 20° C. for 16 hours. The reaction mixture was quenched with water (30 mL) and extracted with DCM (30 mL×3). The combined organic layer was washed with brine (30 mL), dried over anhydrous Na₂SO₄, filtered and concentrated. The residue was purified by Combi-Flash (0% to 30% EtOAc in PE) to give 5-bromo-3,3-dimethylisobenzofuran-1(3H)-one (350 mg, yield: 31%) as a light yellow solid.

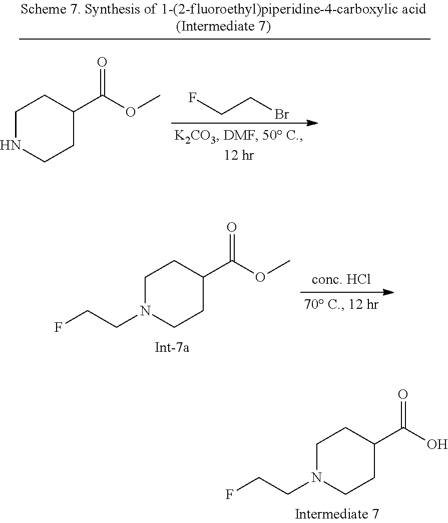

### Step 1. Synthesis of methyl 1-(2-fluoroethyl)piperidine-4-carboxylate

A mixture of methyl piperidine-4-carboxylate (200 mg, 1.40 mmol), 1-bromo-2-fluoro-ethane (266 mg, 2.10 mmol) and K₂CO₃ (386 mg, 2.79 mmol) in DMF (10 mL) was stirred at 50° C. for 12 hours. The reaction was filtered and the filtrate was concentrated. The residue was purified by Combi-Flash (0% to 50% EtOAc in PE) to give methyl 1-(2-fluoroethyl)piperidine-4-carboxylate (100 mg, yield: 38%) as colorless oil.

### Step 2. Synthesis of 1-(2-fluoroethyl)piperidine-4-carboxylic acid

A solution of methyl piperidine-4-carboxylate (100 mg, 0.528 mmol) in conc. HCl (2 mL) was stirred at 70° C. for 12 hours. The reaction mixture was concentrated to give 1-(2-fluoroethyl)piperidine-4-carboxylic acid (80 mg, yield: 72%, HCl salt) as a white solid.

### Example 1

### N-(5-(benzo[d]oxazol-6-yl)thiazol-2-yl)-1-methylpiperidine-4-carboxamide

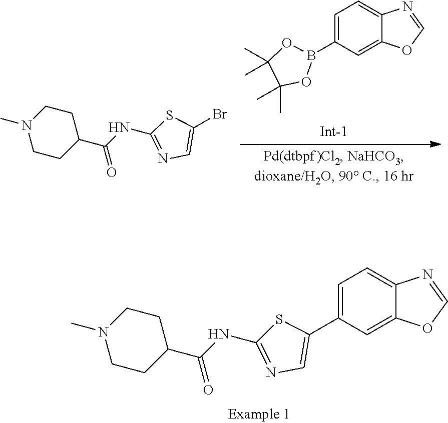 [SMILES: CN1CCC(C(=O)Nc2ncc(Br)s2)CC1 ; CC1(C)OB(c2ccc3ncoc3c2)OC1(C)C ; CN1CCC(C(=O)Nc2ncc(-c3ccc4ncoc4c3)s2)CC1]

A mixture of Intermediate 1 (220 mg, 0.898 mmol), N-(5-bromothiazol-2-yl)-1-methylpiperidine-4-carboxamide (819 mg, 2.69 mmol), Pd(dtbpf)Cl₂ (59 mg, 0.090 mmol), NaHCO₃ (226 mg, 2.69 mmol) in dioxane (4 mL) and H₂O (1 mL) was degassed and purged with N₂ for 3 times, and then the mixture was stirred at 90° C. for 16 hours under N₂ atmosphere. The reaction mixture was filtered and the filtrate was concentrated. The residue was purified by prep-HPLC (0.04% NH₃.H₂O+10 mM NH₄HCO₃ as an additive), then lyophilized to give N-(5-(benzo[d]oxazol-6-yl)thiazol-2-yl)-1-methylpiperidine-4-carboxamide (3 mg, yield: 4%) as a white solid.

¹H NMR (400 MHz, CD₃OD) δ 1.82-1.94 (4H, m), 2.10-2.20 (2H, m), 2.32 (3H, s), 2.50-2.59 (1H, m), 2.50-3.00 (2H, m), 7.69 (1H, dd, J=8.4, 1.6 Hz), 7.76-7.82 (2H, m), 7.94 (1H, d, J=1.2 Hz), 8.51 (1H, s).

The following compounds were synthesized analogously to Example 1

| Example | | | |
|---|---|---|---|
| No. | Structure | Name | ¹H NMR (400 MHz) |
| 2 | 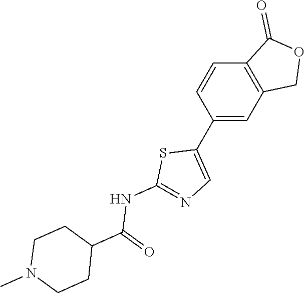 [SMILES: CN1CCC(C(=O)Nc2ncc(-c3ccc4c(c3)COC4=O)s2)CC1] | 1-methyl-N-(5-(1- oxo-1,3- dihydroisobenzofuran- 5-yl)thiazol-2- yl)piperidine-4- carboxamide | DMSO-d₆; δ 1.90-2.20 (4H, m), 2.70-2.75 (2H, m), 2.76- 2.80 (1H, m), 2.87-3.06 (2H, m), 3.40-3.50 (3H, m), 5.42 (2H, s), 7.80-7.85 (2H, m), 7.88 (1H, s), 8.10 (1H, s), 10.39 (1H, brs), 12.53 (1H, brs). |
| 4 | 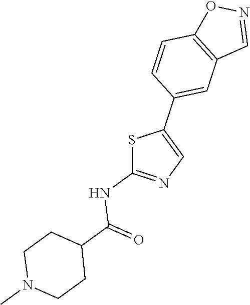 [SMILES: CN1CCC(C(=O)Nc2ncc(-c3ccc4oncc4c3)s2)CC1] | N-(5- (benzo[d]isoxazol-5- yl)thiazol-2-yl)-1- methylpiperidine-4- carboxamide | DMSO-d₆; δ 1.61-1.73 (2H, m), 1.77-1.84 (2H, m), 1.94- 2.03 (2H, m), 2.22 (3H, s), 2.42-2.50 (1H, m), 2.80- 2.92 (2H, m), 7.05 (1H, d, J = 8.4 Hz), 7.68 (1H, dd, J = 8.8, 2.4 Hz), 7.79 (1H, s), 7.87 (1H, d, J = 2.4 Hz), 8.18 (1H, s), 12.15 (1H, brs). |
| 5 | 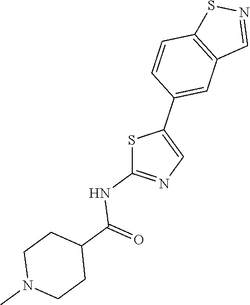 [SMILES: CN1CCC(C(=O)Nc2ncc(-c3ccc4sncc4c3)s2)CC1] | N-(5- (benzo[d]isothiazol-5- yl)thiazol-2-yl)-1- methylpiperidine-4- carboxamide | DMSO-d₆; δ 1.69-1.93 (2H, m), 1.98-2.10 (2H, m), 2.52- 2.56 (4H, m), 2.71-2.97 (4H, m), 7.93 (1H, dd, J = 8.8, 1.6 Hz), 8.03 (1H, s), 8.28 (1H, d, J = 8.4 Hz), 8.41 (1H, s), 9.14 (1H, s), 12.39 (1H, brs). |

### Example 3

### N-(5-(3,3-dimethyl-1-oxo-1,3-dihydroisobenzofuran-5-yl)thiazol-2-yl)-1-methylpiperidine-4-carboxamide

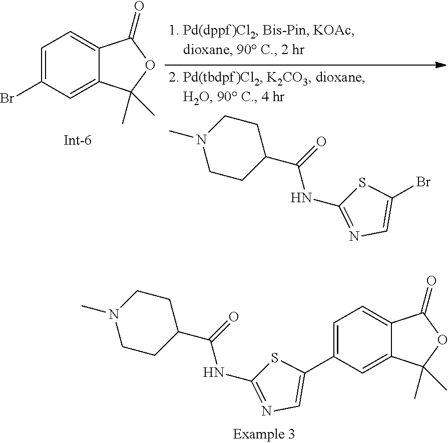

A mixture of Bis-Pin (329 mg, 1.29 mmol), Intermediate 6 (240 mg, 0.996 mmol), Pd(dppf)Cl₂ (73 mg, 0.099 mmol) and KOAc (195 mg, 1.99 mmol) in anhydrous dioxane (4 mL) was degassed and purged with N₂ for 3 times and stirred at 90° C. for 2 hours under N₂ atmosphere. Then a mixture of N-(5-bromothiazol-2-yl)-1-methylpiperidine-4-carboxamide (273 mg, 0.896 mmol), Pd(dtbpf)Cl₂ (65 mg, 0.099 mmol) and K₂CO₃ (275 mg, 1.99 mmol) in anhydrous dioxane (4 mL) and H₂O (0.8 mL) was added to the above mixture under N₂ atmosphere and stirred at 90° C. for another 4 hours. The reaction mixture was filtered through a pad of celite and the filtrate was concentrated. The residue was purified by prep-HPLC (0.025% FA as an additive), then lyophilized to give N-(5-(3,3-dimethyl-1-oxo-1,3-dihydroisobenzofuran-5-yl)thiazol-2-yl)-1-methylpiperidine-4-carboxamide (35 mg, yield: 9%) as a white solid.

¹H NMR (400 MHz, DMSO-d₆) δ 1.59-1.71 (8H, m), 1.75-1.95 (4H, m), 2.15 (3H, s), 2.41-2.48 (1H, m), 2.78-2.81 (2H, m), 7.73-7.84 (2H, m), 8.01 (1H, s), 8.12 (1H, s).

### Example 6

### 1-(2-fluoroethyl)-N-(6-(oxazol-5-yl)pyrrolo[1,2-c]pyrimidin-3-yl)piperidine-4-carboxamide

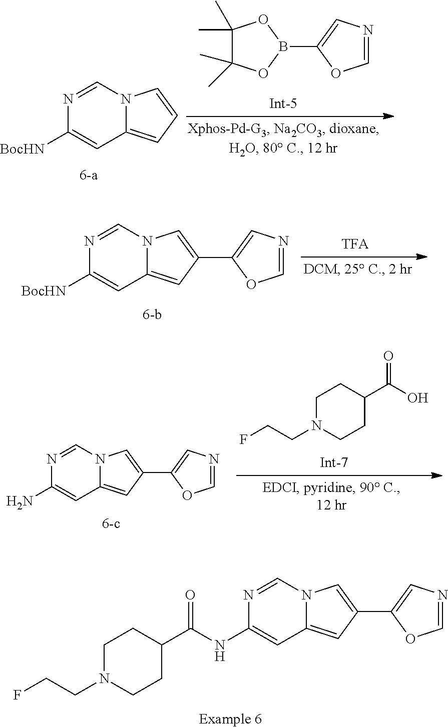

### Step 1. Synthesis of tert-butyl (6-(oxazol-5-yl)pyrrolo[1,2-c]pyrimidin-3-yl)carbamate

A mixture of tert-butyl pyrrolo[1,2-c]pyrimidin-3-ylcarbamate (100 mg, 0.320 mmol), Intermediate 5 (187 mg, 0.961 mmol), Xphos-Pd-G₃ (27 mg, 0.03 mmol) and Na₂CO₃ (68 mg, 0.64 mmol) in 1,4-dioxane (5 mL) and H₂O (1 mL) was degassed and purged with N₂ for 3 times, then the mixture was stirred at 80° C. for 12 hours under N₂ atmosphere. The reaction mixture was diluted with H₂O (20 mL) and extracted with EtOAc (40 mL×3). The combined organic layers were dried over anhydrous Na₂SO₄, filtered and concentrated. The residue was purified by Combi-Flash (0% to 30% EtOAc in PE) to give compound 7 (80 mg, yield: 83%) as a yellow solid.

¹H NMR (400 MHz, DMSO-d₆) δ 1.48 (9H, s), 6.55 (1H, s), 7.51 (1H, s), 7.60 (1H, s), 7.86 (1H, d, J=0.8 Hz), 8.40 (1H, s), 8.92-9.12 (1H, m), 9.57 (1H, brs).

### Step 2. Synthesis of 6-(oxazol-5-yl)pyrrolo[1,2-c]pyrimidin-3-amine

A mixture of tert-butyl (6-(oxazol-5-yl)pyrrolo[1,2-c]pyrimidin-3-yl)carbamate (80 mg, 0.27 mmol) in DCM (4 mL) and TFA (1 mL) was stirred at 25° C. for 2 hours. The reaction mixture was basified with saturated aqueous NaHCO₃ to pH=8 and extracted with MeOH/DCM (25 mL ×2, 1/10), the combined organic layers were dried over anhydrous Na₂SO₄, filtered and concentrated to give 6-(oxazol-5-yl)pyrrolo[1,2-c]pyrimidin-3-amine (30 mg, crude) as a gray solid.

### Step 3. Synthesis of 1-(2-fluoroethyl)-N-(6-(oxazol-5-yl)pyrrolo[1,2-c]pyrimidin-3-yl)piperidine-4-carboxamide

A mixture of Intermediate 7 (32 mg, 0.15 mmol), 6-(oxazol-5-yl)pyrrolo[1,2-c]pyrimidin-3-amine (30 mg, 0.15 mmol) and EDCI (58 mg, 0.30 mmol) in pyridine (3 mL) was stirred at 90° C. for 12 hours. The reaction mixture was basified with saturated aqueous NaHCO₃ to pH =8 and extracted with DCM (25 mL×2). The combined organic layers were dried over anhydrous Na₂SO₄, filtered and concentrated. The residue was purified by prep-HPLC (0.05% NH₃H₂O+10 mM NH₄HCO₃ as an additive) and lyophilized to give 1-(2-fluoroethyl)-N-(6-(oxazol-5-yl)pyrrolo[1,2-c]pyrimidin-3-yl)piperidine-4-carboxamide (2.06 mg, yield: 4%) as a gray solid.

¹H NMR (400 MHz, CD₃OD) δ 1.85-1.97 (4H, m), 2.19-2.29 (2H, m), 2.42-2.54 (1H, m), 2.60-2.80 (2H, m), 3.00-3.20 (2H, m), 4.50-4.70 (2H, m), 6.62 (1H, s), 7.42 (1H, s), 7.84 (1H, s), 7.99 (1H, s), 8.24 (1H, s), 8.87 (1H, s).

### Example 7

### 5-(1-methyl-1H-indazol-6-yl)-2-(piperidin-4-ylethynyl)thiazole

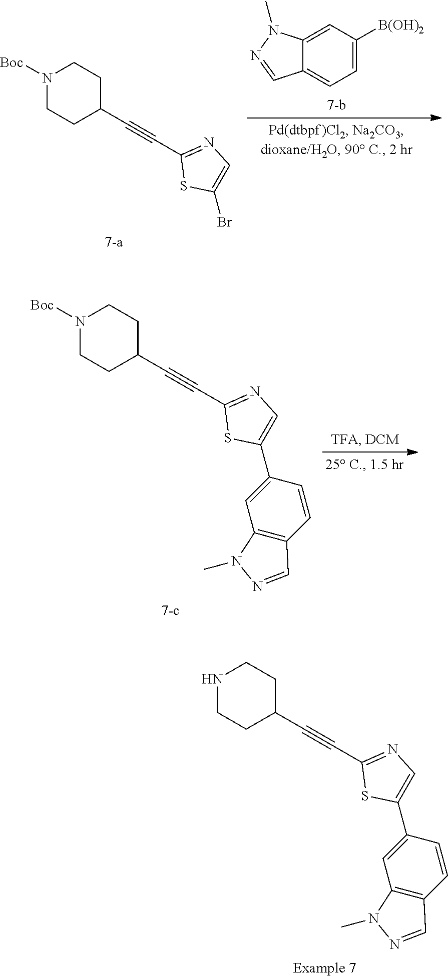 [SMILES: CC(C)(C)OC(=O)N1CCC(C#Cc2ncc(Br)s2)CC1 ; Cn1ncc2ccc(B(O)O)cc21 ; Cn1ncc2ccc(-c3cnc(C#CC4CCN(C(=O)OC(C)(C)C)CC4)s3)cc21 ; Cn1ncc2ccc(-c3cnc(C#CC4CCNCC4)s3)cc21]

### Step 1. Synthesis of tert-butyl 4-((5-(1-methyl-1H-indazol-6-yl)thiazol-2-yl)ethynyl)piperidine-1-carboxylate

A mixture of tert-butyl 4-((5-bromothiazol-2-yl)ethynyl)piperidine-1-carboxylate (500 mg, 1.35 mmol), (1-methyl-1H-indazol-6-yl)boronic acid (284 mg, 1.62 mmol), Pd(dtbpf)Cl₂ (88 mg, 0.13 mmol) and Na₂CO₃ (285 mg, 2.69 mmol) in 1, 4-dioxane (4 mL) and H₂O (0.5 mL) was degassed and purged with N₂ for 3 times and then the mixture was stirred at 90° C. for 2 hours under N₂ atmosphere. The reaction mixture was concentrated and the residue was purified by silica gel column (PE/EtOAc=2/1) to afford tert-butyl 4-((5-(1-methyl-1H-indazol-6-yl)thiazol-2-yl)ethynyl)piperidine-1-carboxylate (220 mg, yield: 35%) as a brown solid.

¹H NMR (400 MHz, DMSO-d₆) δ 1.41 (9H, s), 1.50-1.62 (2H, m), 1.82-1.94 (2H, m), 3.07-3.15 (2H, m), 3.63-3.72 (2H, m), 4.09 (3H, s), 4.13 (1H, s), 7.44 (1H, dd, J=8.4, 1.6 Hz), 7.83-7.85 (1H, m), 8.00-8.04 (1H, m), 8.08 (1H, s), 8.38 (1H, s).

### Step 2. Synthesis of 5-(1-methyl-1H-indazol-6-yl)-2-(piperidin-4-ylethynyl)thiazole

To a solution of tert-butyl 4-((5-(1-methyl-1H-indazol-6-yl)thiazol-2-yl)ethynyl)piperidine-1-carboxylate (220 mg, 0.520 mmol) in DCM (2 mL) was added TFA (2 mL). The mixture was stirred at 20° C. for 1.5 hours. The reaction mixture was concentrated and the residue was purified by prep-HPLC (Method B; 0.05% NH₃H₂O+NH₄HCO₃ as an additive), then lyophilized to afford 5-(1-methyl-1H-indazol-6-yl)-2-(piperidin-4-ylethynyl)thiazole (8.55 mg, yield: 2%) as a white solid.

¹H NMR (400 MHz, DMSO-d₆) δ 1.47-1.59 (2H, m), 1.76-1.87 (2H, m), 2.53-2.60 (2H, m), 2.80-2.94 (3H, m), 4.09 (3H, s), 7.45 (1H, dd, J=8.4, 1.2 Hz), 7.82 (1H, d, J=8.0 Hz), 8.01 (1H, s), 8.08 (1H, s), 8.37 (1H, s).

The following compound was synthesized analogously to Example 7

| Example | | | |
|---|---|---|---|
| No. | Structure | Name | ¹H NMR (400 MHz) |
| 9 | 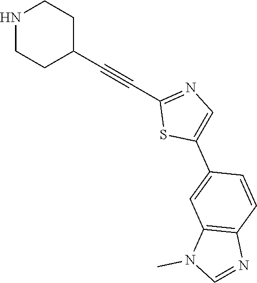 [SMILES: Cn1cnc2ccc(-c3cnc(C#CC4CCNCC4)s3)cc21] | 5-(1-methyl-1H- benzo[d]imidazol- 6-yl)-2-(piperidin- 4-ylethynyl)thiazole | DMSO-d₆; δ 1.47-1.60 (2H, m), 1.77-1.88 (2H, m), 2.52-2.65 (2H, m), 2.75-2.98 (3H, m), 3.88 (3H, s), 7.51 (1H, dd, J = 8.4, 1.6 Hz), 7.70 (1H, d, J = 8.8 Hz), 7.94 (1H, d, J = 1.2 Hz), 8.25 (1H, s), 8.27 (1H, s). |

### Example 8

### 5-(1-methyl-1H-indazol-6-yl)-2-((1-methylpiperidin-4-yl)ethynyl)thiazole

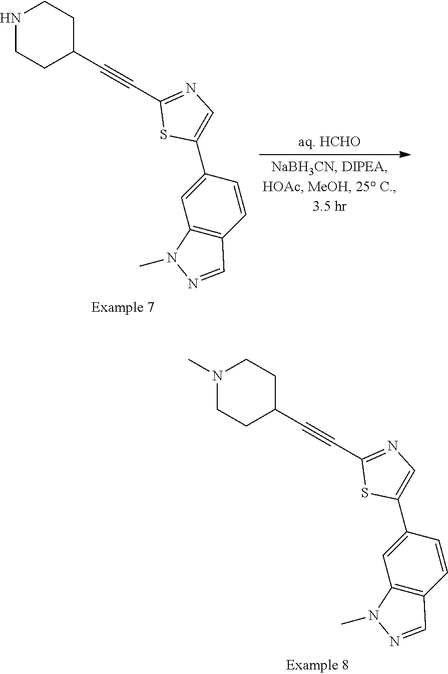 [SMILES: Cn1ncc2ccc(-c3cnc(C#CC4CCNCC4)s3)cc21 ; CN1CCC(C#Cc2ncc(-c3ccc4cnn(C)c4c3)s2)CC1]

To a solution of 5-(1-methyl-1H-indazol-6-yl)-2-(piperidin-4-ylethynyl)thiazole (Example 7; 300 mg, 0.930 mmol) in MeOH (5 mL) was added DIPEA (0.3 mL) at 20° C. and stirred for 0.5 hour, then HOAc (0.2 mL) and HCHO (42 mg, 1.4 mmol) were added to the reaction mixture and stirred for 1 hour. NaBH₃CN (88 mg, 1.4 mmol) was added and the reaction mixture was stirred at 20° C. for another 2 hours. The reaction mixture was concentrated and the residue was purified by prep-HPLC (Method B; 0.05% NH₃H₂O+NH₄HCO₃ as an additive), then lyophilized to afford 5-(1-methyl-TH-indazol-6-yl)-2-((1-methylpiperidin-4-yl)ethynyl)thiazole (26.08 mg, yield: 8%) as a yellow solid.

¹H NMR (400 MHz, DMSO-d₆) δ 1.60-1.72 (2H, m), 1.85-1.94 (2H, m), 2.06-2.14 (2H, m), 2.16 (3H, s), 2.55-2.60 (2H, m), 2.75-2.80 (TH, m), 4.09 (3H, s), 7.45 (TH, dd, J=8.4, 1.6 Hz), 7.82 (1H, d, J=8.0 Hz), 8.01 (1H, s), 8.08 (1H, s), 8.37 (1H, s).

The following compound was synthesized analogously to Example 8

| Example | | | |
|---|---|---|---|
| No. | Structure | Name | ¹H NMR (400 MHz) |
| 10 | 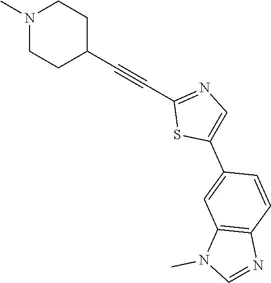 [SMILES: CN1CCC(C#Cc2ncc(-c3ccc4ncn(C)c4c3)s2)CC1] | 5-(1-methyl-1H- benzo[d]imidazol- 6-yl)-2-((1- methylpiperidin-4- yl)ethynyl)thiazole | DMSO-d₆; δ 1.58-1.73 (2H, m), 1.81-1.96 (2H, m), 2.02-2.15 (2H, m), 2.16 (3H, s), 2.55-2.64 (2H, m), 2.69-2.83 (1H, m), 3.88 (3H, s), 7.51 (1H, dd, J = 8.4, 2.0 Hz), 7.70 (1H, d, J = 8.4 Hz), 7.95 (1H, d, J = 1.6 Hz), 8.25 (1H, s), 8.27 (1H, s). |

### Intermediates of Formula (IV)

Schemes below illustrate the preparation of intermediates.

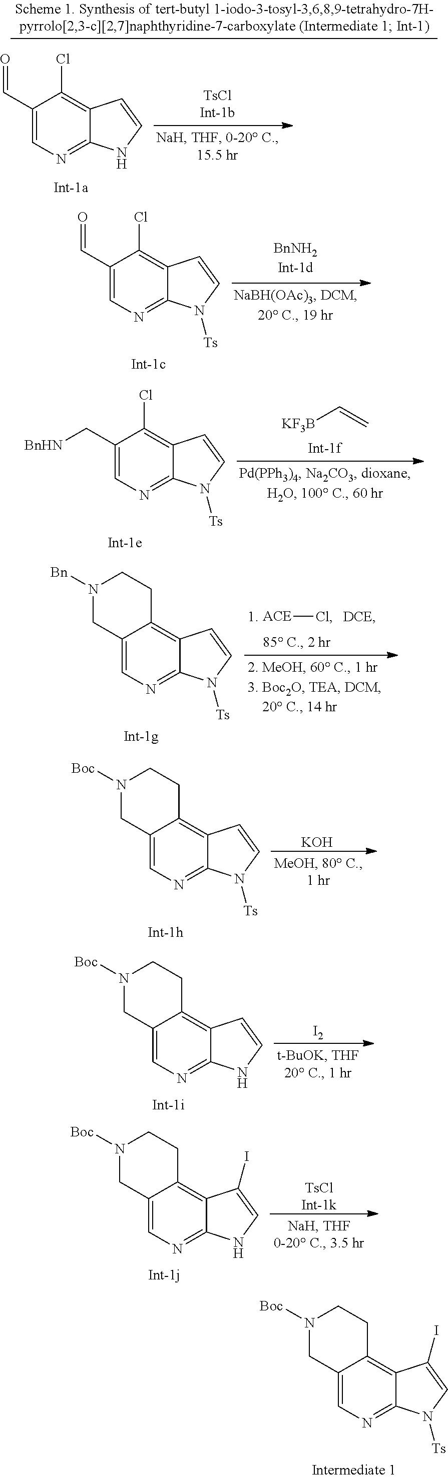

### Step 1. Synthesis of 4-chloro-1-tosyl-1H-pyrrolo[2,3-b]pyridine-5-carbaldehyde

To a solution of compound Int-la (3.50 g, 19.4 mmol) in THF (50 mL) was added NaH (930 mg, 23.3 mmol, 60% dispersion in mineral oil) portion-wise at 0° C. The mixture was stirred at 0° C. for 0.5 hour. TsCl (4.80 g, 25.2 mmol) was added to the mixture at 0° C. and the mixture was stirred at 20° C. for 15 hours. The reaction mixture was quenched with water (200 mL) and the precipitate was filtered. The solid was washed with water (50 mL×3), EtOAc (30 mL×2) and dried to give 4-chloro-1-tosyl-1H-pyrrolo[2,3-b]pyridine-5-carbaldehyde (5.40 g, yield: 83%) as a yellow solid.

¹H NMR (400 MHz, DMSO-d₆) δ 2.35 (3H, s), 7.04 (1H, d, J=4.0 Hz), 7.45 (2H, d, J=8.4 Hz), 8.04 (2H, d, J=8.4 Hz), 8.15 (1H, d, J=4.4 Hz), 8.77 (1H, s), 10.34 (1H, s).

### Step 2. Synthesis of N-benzyl-1-(4-chloro-1-tosyl-1H-pyrrolo[2,3-b]pyridin-5-yl)methanamine

A mixture of compound Int-1c (5.90 g, 17.6 mmol) and BnNH₂ (2.83 g, 26.4 mmol) in DCM (130 mL) was stirred at 20° C. for 4 hours. NaBH(OAc)₃ (7.47 g, 35.3 mmol) was added to the mixture and stirred at 20° C. for 15 hours. The reaction mixture was quenched with water (200 mL) and basified with saturated aqueous NaHCO₃. to pH=8, then extracted with DCM (80 mL×2). The combined organic layer was washed with brine (50 mL), dried over anhydrous Na₂SO₄ and concentrated. The residue was purified by flash silica gel chromatography (ISCO©; 120 g SepaFlash© Silica Flash Column, Eluent of 0˜3% MeOH/DCM @50 mL/min) to give N-benzyl-1-(4-chloro-1-tosyl-1H-pyrrolo[2,3-b]pyridin-5-yl)methanamine (7.10 g, yield: 95%) as colorless gum.

¹H NMR (400 MHz, DMSO-d₆) δ 2.35 (3H, s), 3.72 (2H, s), 3.84 (2H, s), 6.83 (1H, d, J=4.0 Hz), 7.18-7.36 (5H, m), 7.43 (2H, d, J=8.4 Hz), 7.97-8.02 (3H, m), 8.44 (1H, s).

### Step 3. Synthesis of 7-benzyl-3-tosyl-6, 7,8,9-tetrahydro-3H-pyrrolo[2,3-c][2, 7]naphthyridine

A mixture of compound Int-1e (6.40 g, 15.0 mmol), potassium trifluoro(vinyl)boranuide (2.62 g, 19.5 mmol), Pd(PPh₃)₄ (1.74 g, 1.50 mmol) and Na₂CO₃ (4.78 g, 45.1 mmol) in dioxane (100 mL) and H₂O (10 mL) was degassed and purged with N₂ for 3 times, then stirred at 100° C. for 60 hours under N₂ atmosphere. The reaction mixture was diluted with water (300 mL) and extracted with EtOAc (100 mL×3). The combined organic layer was washed with brine (50 mL), dried over anhydrous Na₂SO₄, filtered and concentrated. The residue was purified by flash silica gel chromatography (ISCO®; 120 g SepaFlash® Silica Flash Column, Eluent of 0˜37% Ethyl acetate/Petroleum ether gradient @50 mL/min) to give 7-benzyl-3-tosyl-6,7,8,9-tetrahydro-3H-pyrrolo[2,3-c][2,7]naphthyridine (3.90 g, yield: 62%) as a yellow gum.

¹H NMR (400 MHz, CDCl₃) δ 2.36 (3H, s), 2.81 (2H, t, J=5.6 Hz), 3.00 (2H, t, J=6.0 Hz), 3.68 (2H, s), 3.72 (2H, s), 6.54 (1H, d, J=4.0 Hz), 7.20-7.26 (2H, m), 7.28-7.40 (5H, m), 7.66 (1H, d, J=4.0 Hz), 8.01 (2H, d, J=8.4 Hz), 8.08 (1H, s).

### Step 4. Synthesis of tert-butyl 3-tosyl-3,6,8,9-tetrahydro-7H-pyrrolo[2, 3-c][2, 7]naphthyridine-7-carboxylate

To a solution of compound Int-1g (3.90 g, 9.34 mmol) in DCE (80 mL) was added ACE-C1 (4.01 g, 28.0 mmol), then stirred at 85° C. for 2 hours. The mixture was concentrated and the residue was dissolved in MeOH (80 mL) was stirred at 60° C. for 1 hour, then concentrated. The residue was dissolved in DCM (60 mL), then (Boc)₂₀ (6.20 g, 28.4 mmol) and Et₃N (2.87 g, 28.4 mmol) were added and the resulting reaction mixture was stirred at 20° C. for 14 hours. The mixture was concentrated and the residue was purified by flash silica gel chromatography (ISCO®; 12 g SepaFlash® Silica Flash Column, Eluent of 0˜22% Ethyl acetate/Petroleum ether gradient @30 mL/min) to give tert-butyl 3-tosyl-3,6,8,9-tetrahydro-7H-pyrrolo[2,3-c][2,7]naphthyridine-7-carboxylate (3.20 g, yield: 79% for three steps) as a pale yellow gum.

¹H NMR (400 MHz, CDCl₃) δ 1.49 (9H, s), 2.37 (3H, s), 2.96 (2H, t, J=6.0 Hz), 3.71 (2H, t, J=6.0 Hz), 4.65 (2H, s), 6.56 (1H, d, J=4.0 Hz), 7.23-7.29 (2H, m), 7.70 (1H, d, J=4.0 Hz), 8.05 (2H, d, J=8.4 Hz), 8.19 (1H, s).

### Step 5. Synthesis of tert-butyl 3,6,8,9-tetrahydro-7H-pyrrolo[2,3-c][2, 7]naphthyridine-7-carboxylate

A mixture of compound Int-1h (300 mg, 0.702 mmol) and KOH (118 mg, 2.11 mmol) in EtOH (4.5 mL) and H₂O (1.5 mL) was stirred at 80° C. for 1 hour. The mixture was concentrated and the residue was diluted with saturated aqueous NH₄Cl (20 mL), then extracted with EtOAc (20 mL×2). The combined organic layers were concentrated to give tert-butyl 3,6,8,9-tetrahydro-7H-pyrrolo[2,3-c][2,7]naphthyridine-7-carboxylate (192 mg, crude) as colorless oil.

### Step 6 Synthesis of tert-butyl 1-iodo-3,6,8,9-tetrahydro-7H-pyrrolo[2, 3-c][2, 7]naphthyridine-7-carboxylate

A mixture of compound Int-li (192 mg, 0.702 mmol), 12 (374 mg, 1.48 mmol) and t-BuOK (173 mg, 1.55 mmol) in THF (6 mL) was stirred at 20° C. for 1 hour. The mixture was quenched with saturated aqueous Na₂SO₃ (40 mL) and extracted with EtOAc (20 mL×3). The combined organic layer was washed with brine (10 mL), dried over anhydrous Na₂SO₄, filtered and concentrated to give tert-butyl 1-iodo-3,6,8,9-tetrahydro-7H-pyrrolo[2,3-c][2,7]naphthyridine-7-carboxylate (240 mg, crude) as a yellow gum.

### Step 7. Synthesis of tert-butyl 1-iodo-3-tosyl-3,6,8,9-tetrahydro-7H-pyrrolo[2,3-c][2, 7]naphthyridine-7-carboxylate

To a solution of compound Int-1j (240 mg, 0.601 mmol) in THF (5 mL) was added NaH (31 mg, 0.78 mmol, 60% dispersion in mineral oil) at 0° C. The mixture was stirred at 0° C. for 30 minutes. TsCl (172 mg, 0.902 mmol) was added to the mixture at 0° C. and the mixture was stirred at 20° C. for another 3 hours. The mixture was quenched with saturated aqueous NH₄Cl (20 mL) and extracted with EtOAc (20 mL×3). The combined organic layer was washed with brine (10 mL), dried over anhydrous Na₂SO₄, filtered and concentrated. The residue was purified by flash silica gel chromatography (ISCO®; 4 g SepaFlash® Silica Flash Column, Eluent of 0˜22% Ethyl acetate/Petroleum ether gradient @20 mL/min) to give tert-butyl 1-iodo-3-tosyl-3,6,8,9-tetrahydro-7H-pyrrolo[2,3-c][2,7]naphthyridine-7-carboxylate (200 mg, yield: 60%) was obtained as a colorless gum.

¹H NMR (400 MHz, DMSO-d₆) δ 1.41 (9H, s), 2.34 (3H, s), 3.35-3.41 (2H, m), 3.54-3.65 (2H, m), 4.57 (2H, s), 7.41 (2H, d, J=8.4 Hz), 7.98 (2H, d, J=8.4 Hz), 8.07 (1H, s), 8.23 (1H, s).

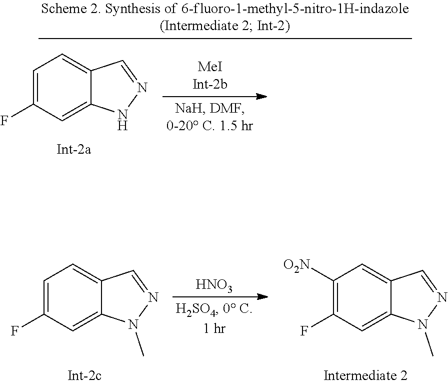

### Step 1. Synthesis of 6-fluoro-1-methyl-1H-indazole

To a solution of compound Int-2a (1.00 g, 7.35 mmol) in DMF (10 mL) was added NaH (353 mg, 8.82 mmol, 60% dispersion in mineral oil) at 0° C. The mixture was stirred at 0° C. for 0.5 hour. Then a solution of MeI (2.09 g, 14.7 mmol) in DMF (5 mL) was added to the mixture slowly and stirred at 0° C. for another 0.5 hour. Then the reaction mixture was stirred at 20° C. for 0.5 hour. The reaction mixture was quenched with H₂O (50 mL) and extracted with EtOAc (60 mL×3). The combined organic layers were washed with brine (80 mL), dried over anhydrous Na₂SO₄, filtered and concentrated. The residue was purified by silica gel column (0% to 20% EtOAc in PE) to give 6-fluoro-1-methyl-1H-indazole (600 mg, yield: 54%) as a yellow solid.

¹H NMR (400 MHz, CDCl₃) δ 4.05 (3H, s), 6.88-6.99 (1H, m), 7.05 (1H, d, J=9.2 Hz), 7.64-7.72 (1H, m), 7.97 (1H, s).

### Step 2. Synthesis of 6-fluoro-1-methyl-5-nitro-1H-indazole

To a solution of compound Int-2c (600 mg, 4.00 mmol) in conc. H₂SO₄ (6 mL) was added conc. HNO₃ (741 mg, 7.99 mmol) slowly. The mixture was stirred at 0° C. for 1 hour. The reaction mixture was slowly poured into ice-water (30 mL) and filtered. The solid was washed with water (5 mL×2) and dried to give 6-fluoro-1-methyl-5-nitro-1H-indazole (300 mg, yield: 38%) as a yellow solid.

¹H NMR (400 MHz, DMSO-d₆) δ 4.08 (3H, s), 7.93 (1H, dd, J=12.4, 0.8 Hz), 8.37 (1H, s), 8.77 (1H, d, J=7.2 Hz).

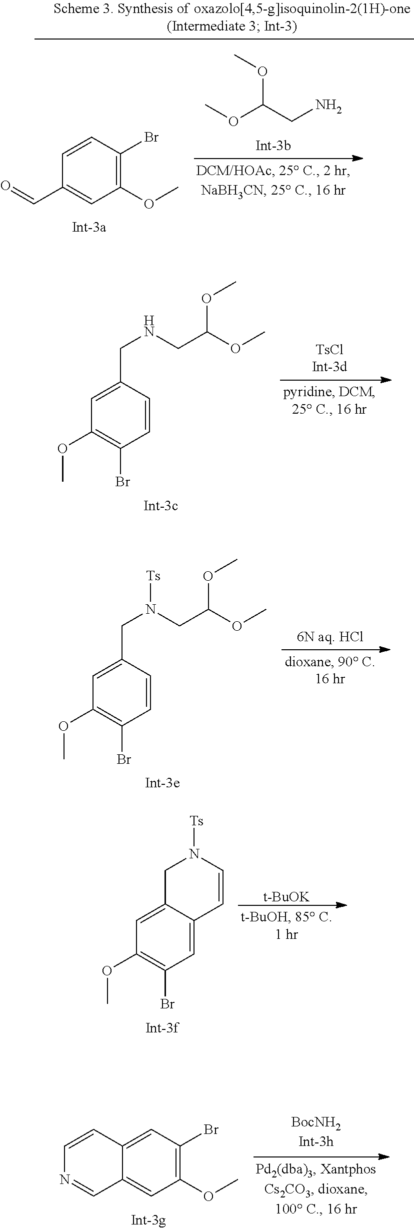

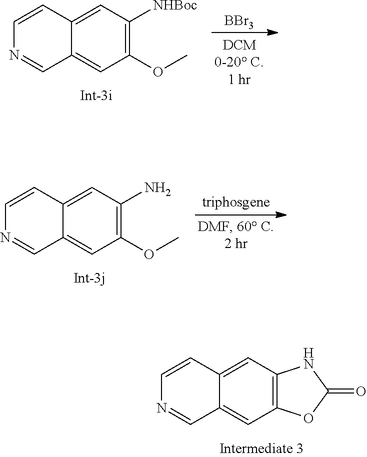 [SMILES: COc1cc2cnccc2cc1NC(=O)OC(C)(C)C ; "BBr3" ; COc1cc2cnccc2cc1N ; O=c1[nH]c2cc3ccncc3cc2o1]

### Step 1. Synthesis of N-(4-bromo-3-methoxybenzyl)-2,2-dimethoxyethan-1-amine

To a solution of compound Int-3a (5.00 g, 23.3 mmol), 2, 2-dimethoxyethylamine (2.93 g, 27.9 mmol) and HOAc (0.5 mL) in DCM (40 mL) was stirred at 25° C. for 2 hours. NaBH₃CN (4.38 g, 69.8 mmol) was added in portions and stirred at 25° C. for another 16 hours. The reaction mixture was diluted with H₂O (50 mL) and extracted with DCM (50 mL×2). The combined organic layer was dried over anhydrous Na₂SO₄, filtered and concentrated. The residue was purified by Combi Flash (0% to 20% MeOH in DCM) to give N-(4-bromo-3-methoxybenzyl)-2,2-dimethoxyethan-1-amine (6.34 g, yield: 90%) as a yellow oil.

### Step 2. Synthesis of N-(4-bromo-3-methoxybenzyl)-N-(2,2-dimethoxyethyl)-4-methylbenzenesulfonamide

To a solution of compound Int-3c (6.34 g, 20.8 mmol) and pyridine (6.59 g, 83.4 mmol) in DCM (50 mL) was added TsCl (5.96 g, 31.3 mmol), then the resulting reaction mixture was stirred at 25° C. for 16 hours. The reaction mixture was washed with 1N aqueous HCl (25 mL×2), dried over anhydrous Na₂SO₄, filtered and concentrated. The residue was purified by Combi Flash (0% to 30% EtOAc in PE) to give N-(4-bromo-3-methoxybenzyl)-N-(2,2-dimethoxyethyl)-4-methylbenzenesulfonamide (7.12 g, yield: 74%) as a colorless oil.

### Step 3. Synthesis of 6-bromo-7-methoxy-2-tosyl-1,2-dihydroisoquinoline

A mixture of compound Int-3e (6.52 g, 14.2 mmol) and 6N aqueous HCl (19 mL) in 1, 4-dioxane (40 mL) was stirred at 90° C. for 16 hours. The reaction mixture was poured into ice water (50 mL) and extracted with DCM (50 mL×3). The combined organic layers was dried over anhydrous Na₂SO₄, filtered and concentrated to give 6-bromo-7-methoxy-2-tosyl-1,2-dihydroisoquinoline (4.89 g, crude) as a yellow solid.

### Step 4. Synthesis of 6-bromo-7-methoxyisoquinoline

A mixture of compound Int-3f (4.89 g, 12.4 mmol) and t-BuOK (6.95 g, 62.0 mmol) in t-BuOH (20 mL) was stirred at 85° C. for 1 hour. The reaction mixture was poured into water (50 mL), and extracted with EtOAc (50 mL×3). The combined organic layers were dried over anhydrous Na₂SO₄, filtered and concentrated. The residue was purified by Combi Flash (0% to 70% EtOAc in PE) to give 6-bromo-7-methoxyisoquinoline (2.19 g, yield: 74%) as a white solid.

### Step 5. Synthesis of tert-butyl (7-methoxyisoquinolin-6-yl)carbamate

A mixture of compound Int-3g (2.14 g, 8.99 mmol), BocNH₂ (1.26 g, 10.8 mmol), Pd₂(dba)₃ (823 mg, 0.899 mmol), Xantphos (1.04 g, 1.80 mmol) and Cs₂CO₃ (5.86 g, 18.0 mmol) in 1,4-dioxane (30 mL) was degassed and purged with N₂ for 3 times, then the mixture was stirred at 100° C. for 16 hours under N₂ atmosphere. The reaction mixture was diluted with H₂O (50 mL) and extracted with EtOAc (50 mL×3). The combined organic layers were dried over anhydrous Na₂SO₄, filtered and concentrated. The residue was purified by Combi Flash (0% to 100% EtOAc in PE) to give tert-butyl (7-methoxyisoquinolin-6-yl)carbamate (2.24 g, yield: 91%) as a brown solid.

### Step 6. Synthesis of 6-aminoisoquinolin-7-ol

To a solution of compound Int-3i (1.00 g, 3.65 mmol) in DCM (20 mL) was added BBr₃ (18.3 g, 72.9 mmol) at 0° C., then the mixture was stirred at 20° C. for 1 hour. The reaction mixture was quenched by addition MeOH (150 mL) at −60° C., then concentrated. The residue was purified by prep-HPLC (0.05% HCl as an additive) and lyophilized to give 6-aminoisoquinolin-7-ol (600 mg, yield: 84%, HCl salt) as a light yellow solid.

¹H NMR (400 MHz, DMSO-d₆) δ 6.90-7.10 (2H, m), 7.43 (1H, d, J=3.2 Hz), 7.77 (1H, d, J=6.8 Hz), 7.98-8.22 (1H, m), 9.07 (1H, d, J=4.8 Hz), 11.34 (1H, brs), 14.38 (1H, brs).

### Step 7. Synthesis of oxazolo[4,5-g]isoquinolin-2(1H)-one

A mixture of compound Int-3j (480 mg, 3.00 mmol) and triphosgene (1.46 g, 8.99 mmol) in DMF (8 mL) was stirred at 60° C. for 2 hours under N₂ atmosphere. The precipitate was filtered and dried to give oxazolo[4,5-g]isoquinolin-2(1H)-one (410 mg, yield: 73%) as a white solid.

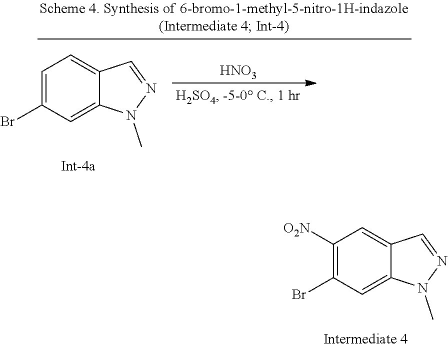

To a solution of compound Int-4a (4.50 g, 21.3 mmol) in conc. H₂SO₄ (40 mL) was added conc. HNO₃ (3.95 g, 42.6 mmol) slowly at −5° C. Then the mixture was stirred at 0° C. for 1 hour. The reaction mixture was slowly poured into ice-water (500 mL) and filtered. The solid was washed with water (20 mL×2) and dried. The residue was purified by silica gel column (0% to 30% EtOAc in PE) to give 6-bromo-1-methyl-5-nitro-1H-indazole (1.80 g, yield: 33%) as a yellow solid.

¹H NMR (400 MHz, DMSO-d₆) δ 4.11 (3H, s), 8.31-8.36 (2H, m), 8.61 (1H, s).

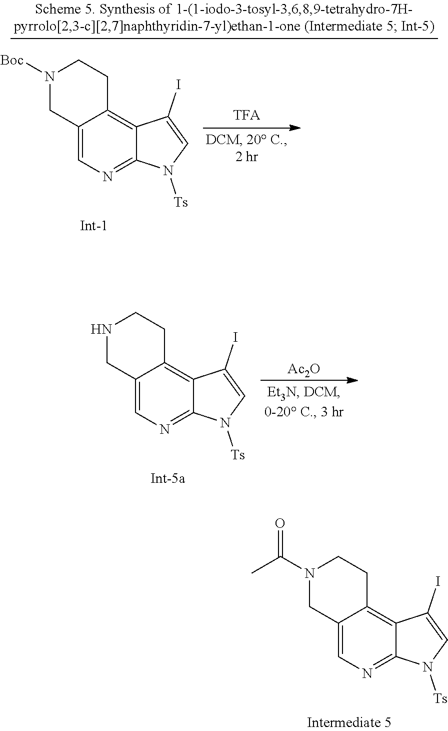 [SMILES: "Ac2O" ; Cc1ccc(S(=O)(=O)n2cc(I)c3c4c(cnc32)CN(C(=O)OC(C)(C)C)CC4)cc1 ; Cc1ccc(S(=O)(=O)n2cc(I)c3c4c(cnc32)CNCC4)cc1 ; CC(=O)N1CCc2c(cnc3c2c(I)cn3S(=O)(=O)c2ccc(C)cc2)C1]

### Step 1. Synthesis of 1-iodo-3-tosyl-6, 7,8,9-tetrahydro-3H-pyrrolo[2, 3-c][2, 7]naphthyridine

To a solution of compound Int-1 (2.00 g, 3.61 mmo) in DCM (20 mL) was added TFA (4 mL) and the reaction mixture was stirred at 20° C. for 2 hours. The reaction mixture was basified with saturated aqueous NaHCO₃ to pH=8 and extracted with EtOAc (50 mL×3). The combined organic layer was dried over anhydrous Na₂SO₄, filtered and concentrated to give 1-iodo-3-tosyl-6,7,8,9-tetrahydro-3H-pyrrolo[2,3-c][2,7]naphthyridine (1.40 g, yield: 85%) as a yellow solid.

### Step 2. Synthesis of 1-(1-iodo-3-tosyl-3,6,8,9-tetrahydro-7H-pyrrolo[2,3-c][2,7]naphthyridin-7-yl)ethan-1-one

To a solution of compound Int-5a (1.10 g, 2.43 mmol) and Et₃N (737 mg, 7.28 mmol) in DCM (15 mL) was added acetyl chloride (229 mg, 2.91 mmol) at 0° C. The reaction mixture was stirred at 20° C. for 3 hours. The reaction mixture was poured into saturated aqueous NaHCO₃ (50 mL) and extracted with EtOAc (50 mL×4). The combined organic layer was dried over anhydrous Na₂SO₄, filtered and concentrated. The crude product was triturated with PE (20 mL) to give 1-(1-iodo-3-tosyl-3,6,8,9-tetrahydro-7H-pyrrolo[2,3-c][2,7]naphthyridin-7-yl)ethan-1-one (1.10 g, yield: 92%) as a yellow solid.

¹H NMR (400 MHz, DMSO-d₆) δ 2.04-2.13 (3H, m), 2.34 (3H, s), 3.05-3.13 (1H, m), 3.45-3.50 (1H, m), 3.65-3.75 (2H, m), 4.65-4.75 (2H, m), 7.42 (2H, d, J=8.4 Hz), 7.94-8.02 (2H, m), 8.06-8.10 (1H, m), 8.21-8.28 (1H, m).

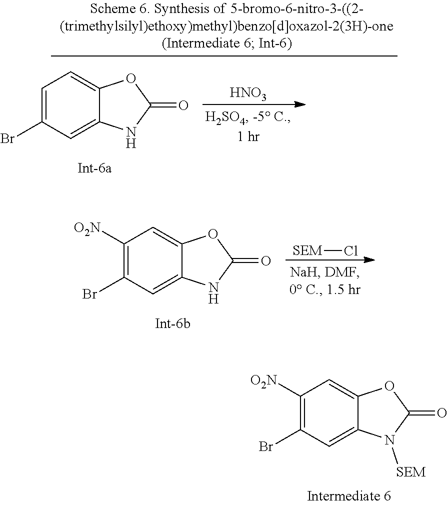

### Step 1. Synthesis of 5-bromo-6-nitrobenzo[d]oxazol-2(3H)-one

To a solution of compound Int-6a (5.00 g, 23.4 mmol) in conc. H₂SO₄ (50 mL) was slowly added conc. HNO₃ (4.33 g, 46.7 mmol) at −5° C., then the mixture was stirred at −5° C. for 1 hour. The reaction mixture was slowly poured into ice-water (200 mL) and filtered. The solid was washed with water (10 mL×2) and dried to give 5-bromo-6-nitrobenzo[d]oxazol-2(3H)-one (5.00 g, yield: 83%) as a gray solid.

¹H NMR (400 MHz, DMSO-d₆) δ 7.55 (1H, s), 8.16 (1H, s), 12.45 (1H, brs).

### Step 2. Synthesis of 5-bromo-6-nitro-3-((2-(trimethylsilyl)ethoxy)methyl)benzo[d]oxazol-2(3H)-one

To a solution of compound Int-6b (3.00 g, 11.6 mmol) in DMF (30 mL) was added NaH (926 mg, 23.2 mmol, 60% dispersion in mineral oil) and stirred at 0° C. for 0.5 hour. Then the SEM-C1 (2.90 g, 17.4 mmol) was added to the mixture and stirred at 0° C. for 1 hour. The reaction mixture was quenched with H₂O (30 mL) and extracted with EtOAc (40 mL×3). The combined organic layers were washed with brine (80 mL), dried over anhydrous Na₂SO₄, filtered and concentrated. The residue was purified by silica gel column (0% to 6% EtOAc in PE) to give 5-bromo-6-nitro-3-((2-(trimethylsilyl)ethoxy)methyl)benzo[d]oxazol-2(3H)-one (1.50 g, crude) as a yellow solid.

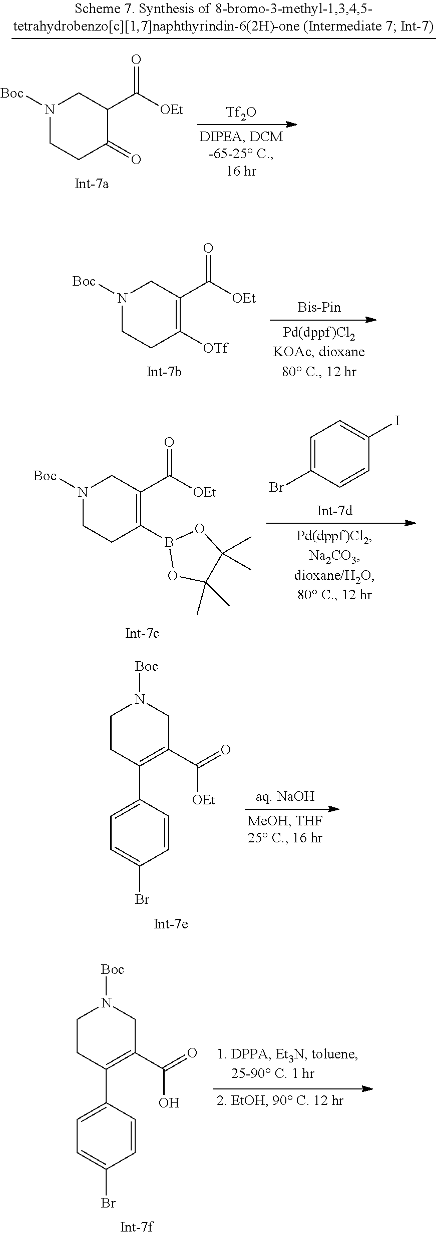

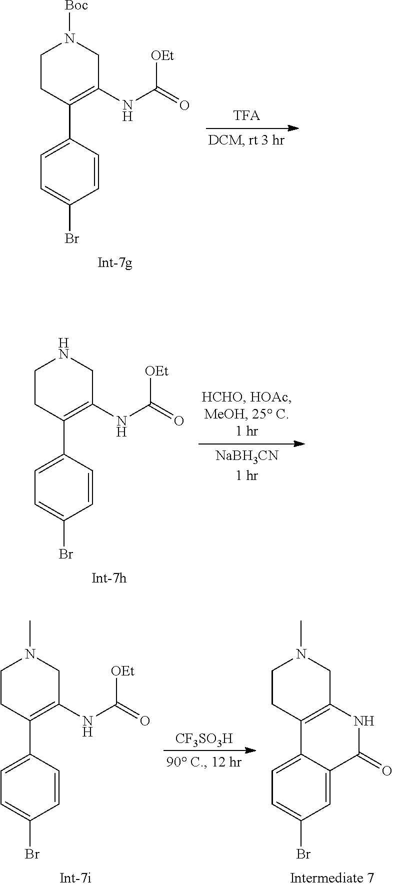

### Step 1. Synthesis of 1-(tert-butyl) 3-ethyl 4-(((trifluoromethyl)sulfonyl)oxy)-5,6-dihydropyridine-1,3(2H)-dicarboxylate

To a solution of compound Int-7a (40.0 g, 147 mmol) in DCM (800 mL) was added DIPEA (22.9 g, 177 mmol) and Tf₂O (45.8 g, 162 mmol) at −65° C., then the reaction was warmed up to 25° C. and stirred for 16 hours. The reaction mixture was quenched by addition saturated aqueous NaHCO₃ (500 mL) and extracted with DCM (500 mL×3). The combined organic layers were washed with brine (250 mL), dried over anhydrous Na₂SO₄, filtered and concentrated. The residue was purified by Combi Flash (0% to 10% EtOAc in PE) to give 1-(tert-butyl) 3-ethyl 4-(((trifluoromethyl)sulfonyl)oxy)-5,6-dihydropyridine-1,3(2H)-dicarboxylate (38.0 g, yield: 64%) as a yellow oil.

### Step 2. Synthesis of 1-(tert-butyl) 3-ethyl 4-(4,4,5,5-tetramethyl-1, 3,2-dioxaborolan-2-yl)-5,6-dihydropyridine-1,3(2H)-dicarboxylate

A mixture of compound Int-7b (35.0 g, 86.8 mmol), Bis-Pin (33.0 g, 130 mmol), Pd(dppf)Cl₂ (6.35 g, 8.68 mmol) and KOAc (17.0 g, 174 mmol) in 1,4-dioxane (300 mL) was degassed and purged with N₂ for 3 times, then the mixture was stirred at 80° C. for 12 hours under N₂ atmosphere. The reaction mixture was concentrated and the residue was diluted with H₂O (100 mL), then extracted with EtOAc (300 mL×3). The combined organic layers were washed with brine (100 mL), dried over anhydrous Na₂SO₄, filtered and concentrated to give 1-(tert-butyl) 3-ethyl 4-(4,4,5,5-tetramethyl-1,3,2-dioxaborolan-2-yl)-5,6-dihydropyridine-1,3(2H)-dicarboxylate (28.0 g, yield: 56%) as a brown oil.

### Step 3. Synthesis of 1-(tert-butyl) 3-ethyl 4-(4-bromophenyl)-5,6-dihydropyridine-1,3(2H)-dicarboxylate

A mixture of compound Int-7c (28.0 g, 73.4 mmol), Int-7d (22.9 g, 80.8 mmol), Pd(dppf)Cl₂ (5.37 g, 7.34 mmol) and Na₂CO₃ (15.6 g, 147 mmol) in 1,4-dioxane (350 mL) and H₂O (35 mL) was degassed and purged with N₂ for 3 times, then the mixture was stirred at 80° C. for 12 hours under N₂ atmosphere. The reaction mixture was diluted with H₂O (100 mL) and extracted EtOAc (200 mL×3). The combined organic layers were dried over anhydrous Na₂SO₄, filtered and concentrated. The residue was purified by Combi Flash (0% to 15% EtOAc in PE) to give 1-(tert-butyl) 3-ethyl 4-(4-bromophenyl)-5,6-dihydropyridine-1,3(2H)-dicarboxylate (15.5 g, yield: 51%) as a yellow oil.

### Step 4. Synthesis of 4-(4-bromophenyl)-1-(tert-butoxycarbonyl)-1,2,5,6-tetrahydropyridine-3-carboxylic acid

To a solution of compound Int-7e (15.5 g, 37.8 mmol) in THF (60 mL) and MeOH (60 mL) was added 2N aqueous NaOH (48 mL). The mixture was stirred at 25° C. for 16 hours. The reaction mixture was concentrated and the residue was acidified with 1N aqueous HCl to pH=1, then extracted with EtOAc (300 mL×3). The combined organic layers were dried over anhydrous Na₂SO₄, filtered and concentrated to give 4-(4-bromophenyl)-1-(tert-butoxycarbonyl)-1,2,5,6-tetrahydropyridine-3-carboxylic acid (13.5 g, yield: 47%) as an off-white solid.

### Step 5. Synthesis of tert-butyl 4-(4-bromophenyl)-5-((ethoxycarbonyl)amino)-3,6-dihydropyridine-1(2H)-carboxylate

To a solution of compound Int7f (13.5 g, 36.0 mmol) in toluene (300 mL) was added DPPA (14.6 g, 53.0 mmol) and Et₃N (7.15 g, 70.6 mmol). Then the mixture was stirred at 25° C. for 0.5 hour, then stirred at 90° C. for another 0.5 hour. EtOH (350 mL) was added to the reaction mixture and stirred at 90° C. for 12 hours. The reaction mixture was concentrated and the residue was diluted with saturated aqueous NaHCO₃ (100 mL), then extracted with EtOAc (300 mL×3). The combined organic layers were dried over anhydrous Na₂SO₄, filtered and concentrated. The residue was purified by Combi Flash (0 to 30% EtOAc in PE) to give tert-butyl 4-(4-bromophenyl)-5-((ethoxycarbonyl)amino)-3,6-dihydropyridine-1(2H)-carboxylate (15.0 g, yield: 100%) as a yellow oil.

### Step 6. Synthesis of ethyl (4-(4-bromophenyl)-1,2,5,6-tetrahydropyridin-3-yl)carbamate

A mixture of compound Int-7g (15.0 g, 35.0 mmol) in TFA (35 mL) and DCM (140 mL) was stirred at 25° C. for 3 hours. The reaction mixture was concentrated to give ethyl (4-(4-bromophenyl)-1,2,5,6-tetrahydropyridin-3-yl)carbamate (16.0 g, crude, TFA salt) as a yellow solid.

### Step 7. Synthesis of ethyl (4-(4-bromophenyl)-1-methyl-1,2,5,6-tetrahydropyridin-3-yl)carbamate

To a solution of compound Int-7h (16.0 g, 36.4 mmol) in MeOH (300 mL) was added HOAc (2.19 g, 36.4 mmol) and 37% aqueous formaldehyde (14.8 g, 182 mmol) and stirred at 25° C. for 1 hour. NaBH₃CN (6.87 g, 109 mmol) was added and the resulting reaction mixture was stirred at 25° C. for another 1 hour. The reaction mixture was concentrated and the residue was diluted with saturated aqueous NaHCO₃ (100 mL), then extracted with DCM (200 mL×2). The combined organic layers were dried over anhydrous Na₂SO₄, filtered concentrated. The residue was purified by Combi Flash (0 to 5% MeOH in DCM) to give ethyl (4-(4-bromophenyl)-1-methyl-1,2,5,6-tetrahydropyridin-3-yl)carbamate (3.70 g, yield: 30%) as a white solid.

¹H NMR (400 MHz, CDCl₃) δ 1.22 (3H, t, J=7.2 Hz), 2.43-2.52 (5H, m), 2.68 (2H, t, J=5.6 Hz), 3.51 (2H, s), 4.08 (2H, q, J=7.2 Hz), 5.98 (1H, brs), 7.13 (2H, d, J=8.8 Hz), 7.52 (2H, d, J=8.4 Hz).

### Step 8. Synthesis of 8-bromo-3-methyl-1,3,4,5-tetrahydrobenzo[c][1, 7]naphthyridin-6(2H)-one

A mixture of compound Int-7i (1.00 g, 2.95 mmol) in CF₃SO₃H (25 mL) was stirred at 90° C. for 12 hours. The reaction mixture was poured into ice water (50 mL), then basified with 2N aqueous NaOH to pH=10 and extracted with DCM (100 mL×3). The combined organic layers were dried over anhydrous Na₂SO₄, filtered and concentrated. The residue was purified by Combi Flash (0 to 5% MeOH in DCM) to give 8-bromo-3-methyl-1,3,4,5-tetrahydrobenzo[c][1,7]naphthyridin-6(2H)-one (370 mg, yield: 43%) as a yellow solid.

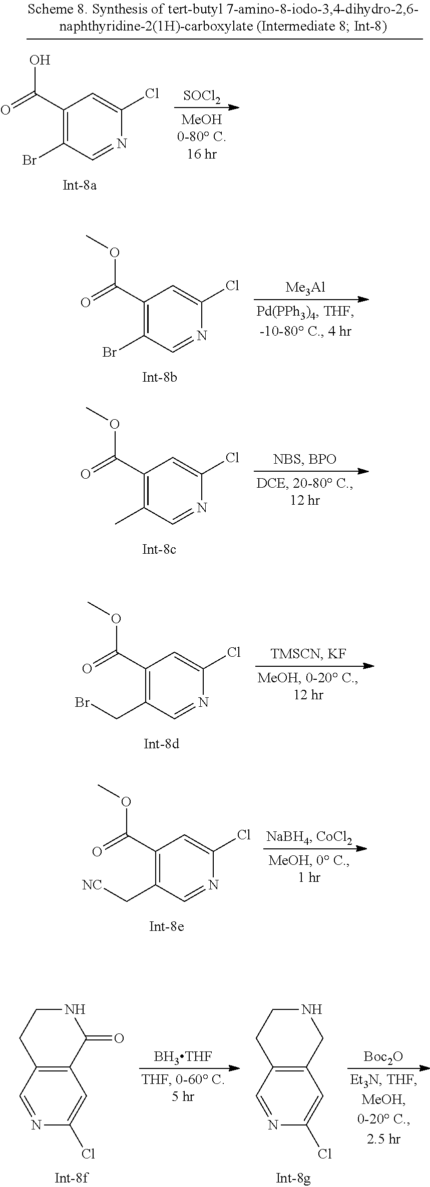

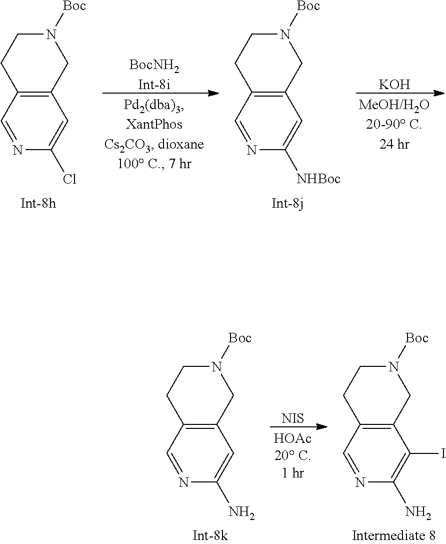

### Step 1. Synthesis of methyl 5-bromo-2-chloroisonicotinate

To a solution of compound Int-8a (50.0 g, 211 mmol) in anhydrous MeOH (500 mL) was added SOCl₂ (20 mL, 276 mmol) dropwise at 0° C. After the addition, the mixture was stirred at 80° C. for 16 hours. The reaction mixture was concentrated and the residue was quenched with saturated aqueous NaHCO₃ (250 mL) and extracted with EtOAc (400 mL×3). The combined organic layer was dried over anhydrous Na₂SO₄, filtered and concentrated to give methyl 5-bromo-2-chloroisonicotinate (52.0 g, yield: 98%) as a brown oil.

### Step 2. Synthesis of methyl 2-chloro-5-methylisonicotinate

To a mixture of compound Int-8b (23.2 g, 92.6 mmol) and Pd(PPh₃)₄ (10.7 g, 9.26 mmol) in anhydrous THF (400 mL) was added AlMe₃ (46 mL, 92.6 mmol, 2M in toluene) dropwise at -10° C. under N₂ atmosphere. The mixture was stirred at −10° C. for 1 hour, followed at 80° C. for another 3 hours. The reaction mixture was quenched with H₂O (10 mL) at 0° C. and concentrated. The residue was purified by Combi Flash (0% to 30% EtOAc in PE) to give methyl 2-chloro-5-methylisonicotinate (9.76 g, yield: 54%) as a light yellow oil.

### Step 3. Synthesis of methyl 5-(bromomethyl)-2-chloroisonicotinate

To a solution of compound Int-8c (9.76 g, 52.6 mmol) in DCE (120 mL) was added NBS (9.17 g, 51.5 mmol) and BPO (2.55 g, 10.5 mmol) at 20° C. The mixture was stirred at 80° C. for 12 hours under N₂ atmosphere. The reaction mixture was concentrated and the residue was purified by Combi Flash (0% to 7% EtOAc in PE) to give methyl 5-(bromomethyl)-2-chloroisonicotinate (11.7 g, yield: 57%) as a light yellow oil.

¹H NMR (400 MHz, CDCl₃) δ 4.01 (3H, s), 4.87 (2H, s), 7.81 (1H, s), 8.52 (1H, s).

### Step 4. Synthesis of methyl 2-chloro-5-(cyanomethyl)isonicotinate

To a solution of compound Int-8d (11.7 g, 44.2 mmol) in absolute MeOH (100 mL) was added TMSCN (22.9 g, 221 mmol) at 0° C., then KF (12.9 g, 221 mmol) was added to the reaction mixture and stirred at 20° C. for 12 hours. The reaction mixture was concentrated and the residue was purified by Combi Flash (0% to 40% EtOAc in PE) to give methyl 2-chloro-5-(cyanomethyl)isonicotinate (6.50 g, yield: 64%) as a white solid.

### Step 5. Synthesis of 7-chloro-3,4-dihydro-2, 6-naphthyridin-1(2H)-one

To a solution of compound Int-8e (6.50 g, 30.9 mmol) in anhydrous MeOH (100 mL) was added CoCl₂ (24.0 g, 185 mmol) at 0° C., then NaBH₄ (7.09 g, 187 mmol) was added to the reaction mixture in small portions over 0.5 hour at 0° C. and stirred for another 0.5 hour. The reaction mixture was quenched with saturated aqueous NH₄Cl (50 mL) at 0° C., then filtered. The solid was washed with MeOH (50 mL×2) and the filtrate was concentrated. The residue was diluted with water (50 mL) and extracted with DCM (100 mL×3). The combined organic layers were washed with brine (80 mL), dried over Na₂SO₄, filtered and concentrated. The residue was purified by Combi Flash (0% to 40% EtOAc in PE) to give 7-chloro-3,4-dihydro-2,6-naphthyridin-1(2H)-one (2.40 g, yield: 41%) as a light yellow solid.

### Step 6. Synthesis of 7-chloro-1,2,3,4-tetrahydro-2, 6-naphthyridine

To a solution of compound Int-8f (1.50 g, 8.21 mmol) in anhydrous THF (25 mL) was added 1M BH₃·THF (28.8 mL, 28.8 mmol) dropwise at 0° C., then stirred at 60° C. for 5 hours. The reaction mixture was quenched with MeOH (10 mL) at 0° C. and stirred at 20° C. for 1 hour.

The mixture was concentrated and the residue was treated with 4N HCl/MeOH (30 mL) at 0° C. and stirred at 20° C. for 1 hour, then concentrated to give 7-chloro-1,2,3,4-tetrahydro-2,6-naphthyridine (1.86 g, yield: 93%, HCl salt) as a light yellow solid.

### Step 7. Synthesis of tert-butyl 7-chloro-3,4-dihydro-2, 6-naphthyridine-2(1H)-carboxylate

To a mixture of compound Int-8g (1.86 g, 7.70 mmol, HCl salt) in anhydrous THF (20 mL) and MeOH (20 mL) was added Et₃N (6.23 g, 61.6 mmol) at 0° C. The mixture was stirred at 0° C. for 0.5 hour and Boc₂O (3.36 g, 15.40 mmol) was added at 0° C., then stirred at 20° C. for 2 hours. The reaction mixture was concentrated and the residue was diluted with water (20 mL), then extracted with DCM (30 mL×3). The combined organic layer was washed with brine (20 mL), dried over anhydrous Na₂SO₄, filtered and concentrated. The residue was purified by Combi Flash (0% to 10% EtOAc in PE) to give tert-butyl 7-chloro-3,4-dihydro-2,6-naphthyridine-2(1H)-carboxylate (2.15 g, yield: 74%) as a white solid.

### Step 8. Synthesis of tert-butyl 7-((tert-butoxycarbonyl)amino)-3,4-dihydro-2,6-naphthyridine-2(1H)-carboxylate

A mixture of compound Int-8h (1.00 g, 3.72 mmol), BocNH₂ (1.31 g, 11.2 mmol), Pd₂(dba)₃ (341 mg, 0.372 mmol), Xantphos (431 mg, 0.744 mmol) and Cs₂CO₃ (2.42 g, 7.44 mmol) in dioxane (20 mL) was degassed and purged with N₂ for 3 times, then stirred at 100° C. for 7 hours under N₂ atmosphere. The reaction mixture was concentrated and the residue was diluted with water (25 mL), then extracted with EtOAc (25 mL×3). The combined organic layer was washed with brine (25 mL), dried over anhydrous Na₂SO₄, filtered and concentrated. The residue was purified by Combi Flash (0% to 10% EtOAc in PE) to give tert-butyl 7-((tert-butoxycarbonyl)amino)-3,4-dihydro-2,6-naphthyridine-2(1H)-carboxylate (630 mg, yield: 43%) as a light yellow solid.

### Step 9. Synthesis of tert-butyl 7-amino-3,4-dihydro-2, 6-naphthyridine-2(1H)-carboxylate

To a solution of compound Int-8j (630 mg, 1.80 mmol) in MeOH (10 mL) and H₂O (5 mL) was added KOH (1.01 g, 18.0 mmol) at 20° C., then stirred at 90° C. for 24 hours. The reaction mixture was concentrated and the residue was diluted with water (25 mL), then extracted with EtOAc (20 mL×3). The combined organic layer was dried over anhydrous Na₂SO₄, filtered and concentrated. The residue was purified by Combi Flash (0% to 100% EtOAc in PE and 0% to 5% MeOH in DCM) to give tert-butyl 7-amino-3,4-dihydro-2,6-naphthyridine-2(1H)-carboxylate (410 mg, yield: 86%) as a light yellow solid.

### Step 10. Synthesis of tert-butyl 7-amino-8-iodo-3,4-dihydro-2,6-naphthyridine-2(1H)-carboxylate

To a solution of compound Int-8k (410 mg, 1.64 mmol) in HOAc (5 mL) was added NIS (444 mg, 1.97 mmol) and stirred at 20° C. for 1 hour. The mixture was concentrated and the residue was basified with saturated aqueous NaHCO₃ to pH=8, then extracted with DCM (20 mL×3). The combined organic layer was washed with brine (20 mL), dried over anhydrous Na₂SO₄, filtered and concentrated. The residue was purified by Combi Flash (0% to 50% EtOAc in PE) to give tert-butyl 7-amino-8-iodo-3,4-dihydro-2,6-naphthyridine-2(1H)-carboxylate (500 mg, yield: 79%) as a light yellow solid.

¹H NMR (400 MHz, CDCl₃) δ 1.51 (9H, s), 2.71 (2H, t, J=5.6 Hz), 3.61 (2H, t, J=5.2 Hz), 4.40 (2H, s), 5.19 (2H, brs), 7.78 (1H, s).

### Preparation of Compounds of Formula (IV)

### Example 1

### 1-(1-cyclobutyl-3,6,8,9-tetrahydro-7H-pyrrolo[2,3-c][2,7]naphthyridin-7-yl)ethan-1-one

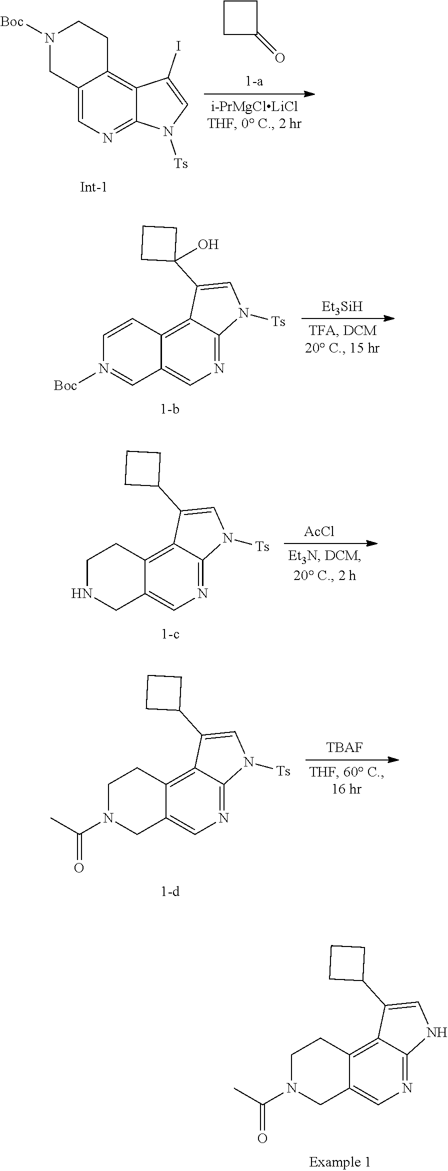

### Step 1. Synthesis of tert-butyl 1-(1-hydroxycyclobutyl)-3-tosyl-3,6,8,9-tetrahydro-7H-pyrrolo[2,3-c][2, 7]naphthyridine-7-carboxylate

To a solution of compound Int-1 (200 mg, 0.361 mmol) in THF (2 mL) was added i-PrMgCl (0.18 mL, 0.36 mmol, 2M in THF) dropwise at 0° C. under N₂. The mixture was stirred at 0° C. for 1 hour. Cyclobutanone (253 mg, 3.61 mmol) was added dropwise to the mixture at 0° C.

The mixture was stirred at 0° C. for another 1 hour. The reaction mixture was quenched with saturated aqueous NH₄Cl (20 mL) and extracted with EtOAc (20 mL×3). The combined organic layer was washed with brine (10 mL), dried over anhydrous Na₂SO₄, filtered and concentrated. The residue was purified by flash silica gel chromatography (ISCO©; 4 g SepaFlash© Silica Flash Column, Eluent of 0˜40% Ethylacetate/Petroleum ether gradient @20 mL/min) to give tert-butyl 1-(1-hydroxycyclobutyl)-3-tosyl-3,6,8,9-tetrahydro-7H-pyrrolo[2,3-c][2,7]naphthyridine-7-carboxylate (70 mg, crude) as a colorless gum.

### Step 2. Synthesis of 1-cyclobutyl-3-tosyl-6,7,8,9-tetrahydro-3H-pyrrolo[2,3-c][2, 7]naphthyridine

A mixture of compound 1-b (70 mg, 0.14 mmol) in DCM (0.4 mL), TFA (0.4 mL) and Et₃SiH (0.4 mL) was stirred at 20° C. for 15 hours. The reaction mixture was quenched with saturated aqueous NaHCO₃ (20 mL) and extracted with DCM (15 mL×2). The combined organic layer was dried over anhydrous Na₂SO₄, filtered and concentrated to give 1-cyclobutyl-3-tosyl-6,7,8,9-tetrahydro-3H-pyrrolo[2,3-c][2,7]naphthyridine (60 mg, crude) as pale yellow gum.

### Step 3. Synthesis of 1-(1-cyclobutyl-3-tosyl-3,6,8,9-tetrahydro-7H-pyrrolo[2,3-c][2,7]naphthyridin-7-yl)ethan-1-one

To a mixture of compound 1-c (200 mg, 0.524 mmol) and Et₃N (159 mg, 1.57 mmol) in DCM (5 mL) was added acetyl chloride (123 mg, 1.57 mmol) at 0° C. and the reaction mixture was stirred at 20° C. for 2 hours. The reaction mixture was poured into brine (10 mL) and extracted with EtOAc (20 mL×3). The combined organic layer was dried over anhydrous Na₂SO₄, filtered and concentrated. The residue was purified by flash column (SiO₂, 0% to 3% MeOH in DCM) to give 1-(1-cyclobutyl-3-tosyl-3,6,8,9-tetrahydro-7H-pyrrolo[2,3-c][2,7]naphthyridin-7-yl)ethan-1-one (140 mg, yield: 63%) as a yellow gum.

### Step 4. Synthesis of 1-(1-cyclobutyl-3,6,8,9-tetrahydro-7H-pyrrolo[2, 3-c][2, 7]naphthyridin-7-yl)ethan-1-one

To a solution of compound 1-d (130 mg, 0.307 mmol) in THF (5 mL) was added TBAF (2 mL, 1M in THF), then stirred at 60° C. for 16 hours. The reaction mixture was poured into saturated aqueous NH₄Cl (20 mL) and extracted with EtOAc (30 mL×3). The combined organic phase washed with saturated aqueous NH₄Cl (20 mL×5), dried over Na₂SO₄, filtered and concentrated. The residue was purified by prep-HPLC (Method C; 0.225% FA as an additive) and lyophilized to give the title compound (19.80 mg, yield: 24%) as a yellow solid.

¹H NMR (400 MHz, CD₃OD) δ 1.87-1.95 (1H, m), 2.05-2.20 (4H, m), 2.21 (3H, s), 2.37-2.45 (2H, m), 3.20-3.25 (1H, m), 3.82-3.836 (3H, m), 4.75-4.80 (2H, m), 7.19 (1H, s), 7.95-8.05 (1H, m).

### Examples 2 and 3

### 7-(trans-3-hydroxycyclohexyl)-1-methyl-5,7-dihydroimidazo[4,5-f]indazol-6(1H)-one (Ex. 2) and 7-(cis-3-hydroxycyclohexyl)-1-methyl-5,7-dihydroimidazo[4,5-f]indazol-6(1H)-one (Ex. 3)

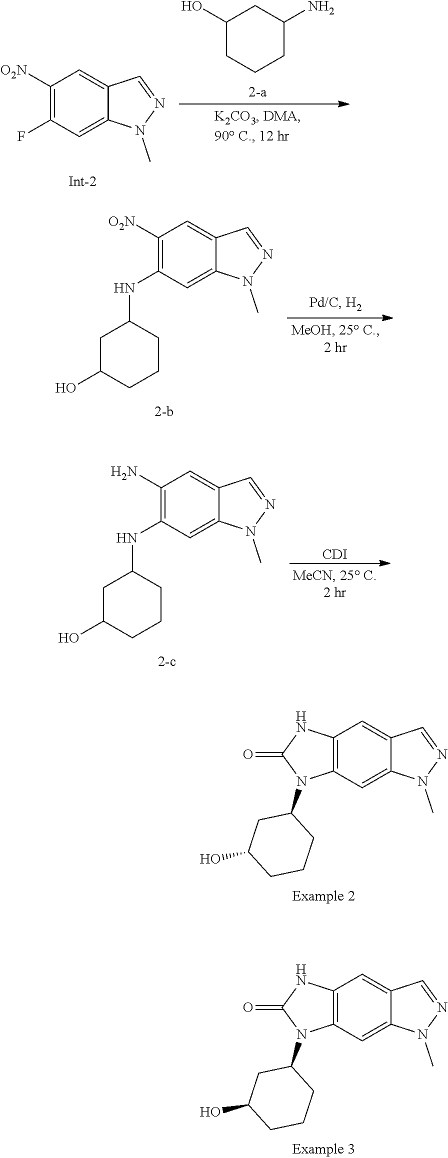

### Step 1. Synthesis of 3-((1-methyl-5-nitro-1H-indazol-6-yl)amino)cyclohexan-1-ol

To a solution of compound Int-2 (210 mg, 1.08 mmol), compound 2-a (186 mg, 1.61 mmol) in DMA (4 mL) was added K₂CO₃ (446 mg, 3.23 mmol). The mixture was stirred at 90° C. for 12 hours. The reaction mixture was diluted with H₂O (20 mL) and extracted with EtOAc (30 mL×3). The combined organic layers were washed with brine (50 mL), dried over anhydrous Na₂SO₄, filtered and concentrated. The residue was purified by silica gel column (0% to 100% EtOAc in PE) to give 3-((1-methyl-5-nitro-1H-indazol-6-yl)amino)cyclohexan-1-ol (200 mg, yield: 56%) as a brown solid.

### Step 2. Synthesis of 3-((5-amino-1-methyl-1H-indazol-6-yl)amino)cyclohexan-1-ol

To a solution of compound 2-b (180 mg, 0.620 mmol) in MeOH (3 mL) was added 10% Pd/C (100 mg). Then the mixture was degassed and purged with H₂ for 3 times and hydrogenated (15 psi) at 25° C. for 2 hours. The reaction mixture was filtered and washed with MeOH (10 mL×3), the filtrate was concentrated to give 3-((5-amino-1-methyl-1H-indazol-6-yl)amino)cyclohexan-1-ol (170 mg, crude) as a brown solid.

### Step 3. Synthesis of 7-(trans-3-hydroxycyclohexyl)-1-methyl-5, 7-dihydroimidazo[4,5-f]indazol-6(1H)-one and 7-(cis-3-hydroxycyclohexyl)-1-methyl-5,7-dihydroimidazo[4,5-f]indazol-6(1H)-one

To a solution of compound 2-c (140 mg, 0.538 mmol) in MeCN (2 mL) was added CDI (174 mg, 1.08 mmol). The mixture was stirred at 25° C. for 2 hours. The reaction mixture was concentrated and the residue was diluted with H₂O (20 mL), then extracted with EtOAc (30 mL×3). The combined organic layers were washed with brine (50 mL), dried over anhydrous Na₂SO₄, filtered and concentrated. The residue was purified by prep-HPLC (Method C; 0.225% FA as an additive), then lyophilized to afford Example 2 (9.53 mg, yield: 5%, trans isomer; stereochemistry as drawn assigned arbitrarily) as a gray solid and Example 3 (8.31 mg, yield: 5%, cis isomer; stereochemistry as drawn assigned arbitrarily) as a gray solid.

Example 2: ¹H NMR (400 MHz, CD₃OD) δ 1.49-1.68 (2H, m), 1.70-1.95 (4H, m), 2.21-2.35 (1H, m), 2.39-2.55 (1H, m), 3.96 (3H, s), 4.16-4.27 (1H, m), 4.53-4.64 (1H, m), 7.17 (1H, s), 7.20 (1H, s), 7.81 (1H, s).

Example 3: ¹H NMR (400 MHz, CD₃OD) δ 1.38-1.61 (2H, m), 1.75-1.88 (1H, m), 1.91-2.01 (1H, m), 2.02-2.15 (2H, m), 2.22-2.40 (2H, m), 3.70-3.84 (1H, m), 4.09 (3H, s), 4.27-4.44 (1H, m), 7.31 (1H, s), 7.34 (1H, s), 7.94 (1H, s).

### Example 4

### 1-(2-(1H-benzo[d]imidazol-2-yl)ethyl)oxazolo[4,5-2]isoquinolin-2(1H)-one

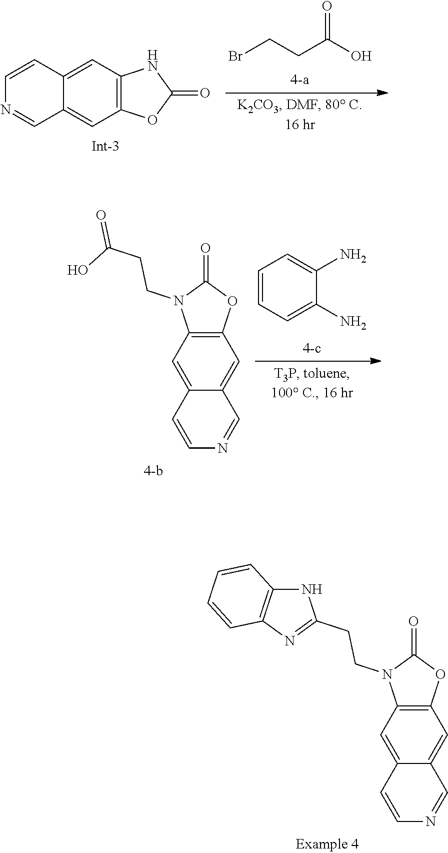

### Step 1. Synthesis of 3-(2-oxooxazolo[4,5-g]isoquinolin-1(2H)-yl)propanoic acid

A mixture of compound Int-3 (200 mg, 1.07 mmol), 3-bromopropanoic acid (197 mg, 1.29 mmol) and K₂CO₃ (445 mg, 3.22 mmol) in DMF (3 mL) was stirred at 80° C. for 16 hours. The reaction mixture was filtered and the filtrate was concentrated. The residue was purified by prep-HPLC (0.04% HCl as an additive) and lyophilized to give 3-(2-oxooxazolo[4,5-g]isoquinolin-1(2H)-yl)propanoic acid (70 mg, yield: 25%) as a white solid.

¹H NMR (400 MHz, DMSO-d₆) δ 2.85 (2H, t, J=6.8 Hz), 4.17 (2H, t, J=7.2 Hz), 8.13 (1H, s), 8.31-8.36 (2H, m), 8.60 (1H, d, J=6.4 Hz), 9.68 (1H, s), 12.53 (1H, brs).

### Step 2. Synthesis of 1-(2-(1H-benzo[d]imidazol-2-yl)ethyl)oxazolo[4,5-g]isoquinolin-2(1H)-one

A mixture of compound 4-b (70 mg, 0.27 mmol), benzene-1, 2-diamine (32 mg, 0.30 mmol), T₃P (259 mg, 0.407 mmol, 50% in EtOAc) in toluene (4 mL) was stirred at 100° C. for 16 hours. The reaction mixture was concentrated under reduced pressure. The residue was diluted with H₂O (10 mL), then basified with saturated aqueous Na₂CO₃ to pH=9 and extracted with EtOAc (20 mL×3). The combined organic layers were dried over anhydrous Na₂SO₄, filtered and concentrated. The residue was purified by prep-HPLC (0.05% NH₃.H₂O as an additive), then lyophilized to give the title compound (35.4 mg, yield: 37%) as a light yellow solid.

¹H NMR (400 MHz, DMSO-d₆) δ 3.37-3.41 (2H, m), 4.38 (2H, t, J=7.2 Hz), 7.08-7.15 (2H, m), 7.36-7.41 (1H, m), 7.51-7.56 (1H, m), 7.63 (1H, s), 7.66 (1H, d, J=6.0 Hz), 8.00 (1H, s), 8.41 (1H, d, J=5.6 Hz), 9.23 (1H, s), 12.35 (1H, brs).

### Example 5

### 7-(3-hydroxyphenyl)-1-methyl-5,7-dihydroimidazo[4,5-f]indazol-6(1H)-one

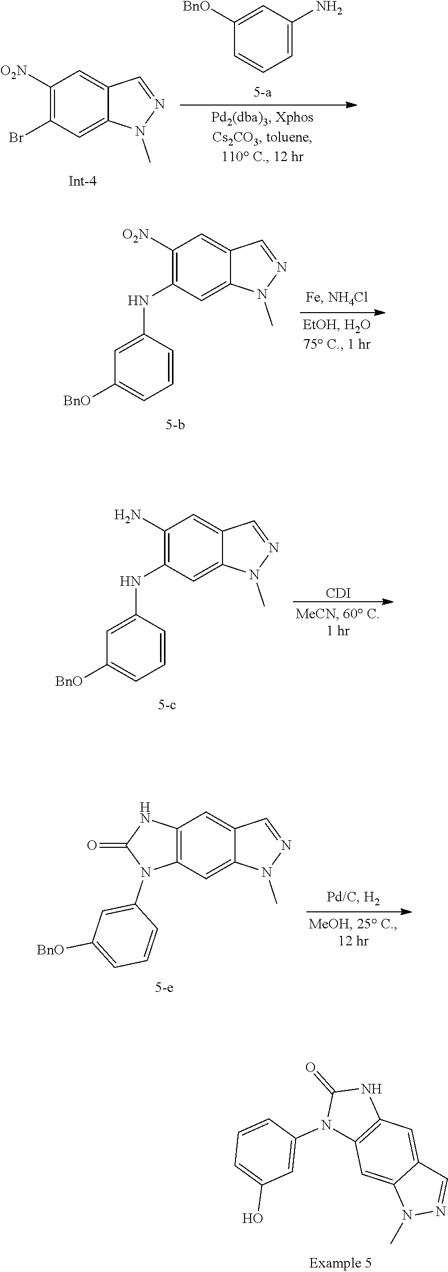

### Step 1. Synthesis of N-(3-(benzyloxy)phenyl)-1-methyl-5-nitro-1H-indazol-6-amine

To a solution of compound Int-4 (500 mg, 1.95 mmol) and compound 5-a (428 mg, 2.15 mmol) in toluene (10 mL) was added Pd₂(dba)₃ (72 mg, 0.078 mmol), Cs₂CO₃ (954 mg, 2.93 mmol) and XPhos (74 mg, 0.16 mmol), then the mixture was degassed and purged with N₂ for 3 times and the mixture was stirred at 110° C. for 12 hours under N₂ atmosphere. The reaction mixture was concentrated and the residue was diluted with H₂O (50 mL), then extracted with EtOAc (80 mL×3). The combined organic layers were washed with brine (80 mL), dried over anhydrous Na₂SO₄, filtered and concentrated. The residue was purified by silica gel column (0% to 50% EtOAc in PE) to give N-(3-(benzyloxy)phenyl)-1-methyl-5-nitro-1H-indazol-6-amine (300 mg, yield: 37%) as a yellow solid.

¹H NMR (400 MHz, DMSO-d₆) δ 3.89 (3H, s), 5.11 (2H, s), 6.71-6.78 (1H, m), 6.93-6.98 (1H, m), 6.99-7.03 (1H, m), 7.25-7.31 (2H, m), 7.32-7.36 (1H, m), 7.37-7.42 (2H, m), 7.43-7.49 (2H, m), 8.18 (1H, s), 8.71 (1H, s), 8.85 (1H, brs).

### Step 2. Synthesis of N⁶-(3-(benzyloxy)phenyl)-1-methyl-1H-indazole-5,6-diamine

To a solution of compound 5-b (250 mg, 0.668 mmol) in EtOH (3 mL) and H₂O (3 mL) was added NH₄Cl (143 mg, 2.67 mmol) and Fe powder (149 mg, 2.67 mmol). The mixture was stirred at 75° C. for 1 hour. The reaction mixture was suspended in CH₃OH (20 mL) and filtered. The filtrate was concentrated and the residue diluted with H₂O (30 mL) and extracted with EtOAc (50 mL×3). The combined organic layers were washed with brine (80 mL), dried over anhydrous Na₂SO₄, filtered and concentrated. The residue was purified by silica gel column (0% to 50% EtOAc in PE) to give N⁶-(3-(benzyloxy)phenyl)-1-methyl-1H-indazole-5,6-diamine (100 mg, yield: 43%) as a white solid.

### Step 3. Synthesis of 7-(3-(benzyloxy)phenyl)-1-methyl-5, 7-dihydroimidazo[4,5-f]indazol-6(1H)-one

To a solution of compound 5-c (100 mg, 0.290 mmol) in MeCN (2 mL) was added CDI (94 mg, 0.581 mmol). The mixture was stirred at 60° C. for 1 hour. The reaction mixture was concentrated and the residue was diluted with H₂O (10 mL) and extracted with EtOAc (20 mL×3). The combined organic layers were washed with brine (30 mL), dried over anhydrous Na₂SO₄, filtered and concentrated to give 7-(3-(benzyloxy)phenyl)-1-methyl-5,7-dihydroimidazo[4,5-f]indazol-6(1H)-one (100 mg, yield: 93%) as a gray solid.

### Step 4. Synthesis of 7-(3-hydroxyphenyl)-1-methyl-5, 7-dihydroimidazo[4,5-f]indazol-6(1H)-one

To a solution of compound 5-e (100 mg, 0.270 mmol) in MeOH (2 mL) was added 10% Pd/C (100 mg). The mixture was degassed and purged with H₂ for 3 times and hydrogenated (15 psi) at 25° C. for 12 hours. The reaction mixture was filtered and the solid was washed with MeOH (5 mL×4), the filtrate was concentrated. The residue was purified by prep-HPLC (Method C; 0.225% FA as an additive) and lyophilized to afford the title compound (7.03 mg, yield: 9%) as a white solid.

¹H NMR (400 MHz, DMSO-d₆) δ 3.95 (3H, s), 6.87 (1H, dd, J=8.0, 1.6 Hz), 6.94-6.98 (1H, m), 6.98-7.03 (1H, m), 7.07 (1H, s), 7.28 (1H, s), 7.36-7.43 (1H, m), 7.94 (1H, s), 9.83 (1H, brs), 11.05 (1H, brs).

The following compound was synthesized analogously to Example 5

| Example | | | |
|---|---|---|---|
| No. | Structure | Name | ¹H NMR (400 MHz) |
| 11 | 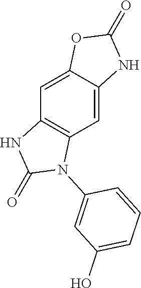 [SMILES: O=c1[nH]c2cc3c(cc2o1)[nH]c(=O)n3-c1cccc(O)c1] | 5-(3-hydroxyphenyl)- 5,7-dihydro-2H- imidazo[4′,5′:4,5]benzo [1,2-d]oxazole- 2,6(3H)-dione | DMSO-d₆; δ 6.65 (1H, s), 6.79- 6.88 (1H, m), 6.88-6.97 (2H, m), 7.08 (1H, s), 7.30-7.40 (1H, m), 9.82 (1H, brs), 11.12 (1H, brs), 11.38 (1H, brs). |

### Example 6

### 1-(1-(benzo[d][1,3]dioxol-5-yl)-3,6,8,9-tetrahydro-7H-pyrrolo[2,3-c][2,7]naphthyridin-7-yl)ethan-1-one

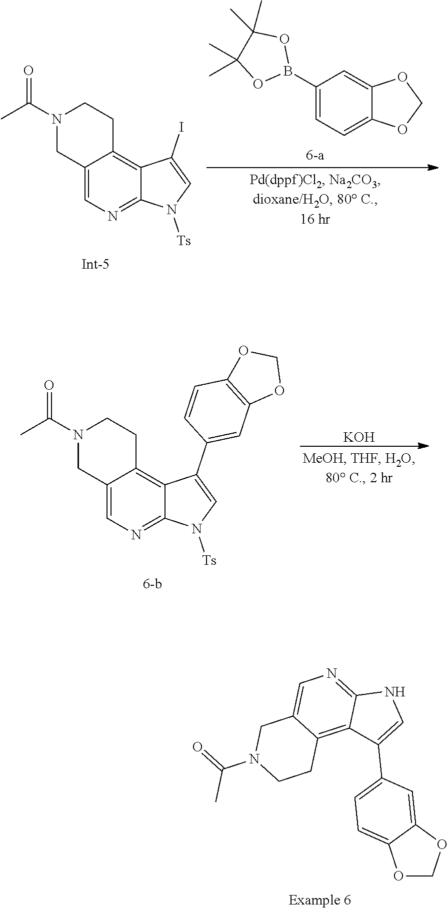 [SMILES: CC(=O)N1CCc2c(cnc3c2c(I)cn3S(=O)(=O)c2ccc(C)cc2)C1 ; CC1(C)OB(c2ccc3c(c2)OCO3)OC1(C)C ; CC(=O)N1CCc2c(cnc3c2c(-c2ccc4c(c2)OCO4)cn3S(=O)(=O)c2ccc(C)cc2)C1 ; "KOH" ; CC(=O)N1CCc2c(cnc3[nH]cc(-c4ccc5c(c4)OCO5)c23)C1]

### Step 1. Synthesis of 1-(1-(benzo[d][1,3]dioxol-5-yl)-3-tosyl-3,6,8,9-tetrahydro-7H-pyrrolo[2,3-c][2, 7]naphthyridin-7-yl)ethan-1-one

A mixture of compound Int-5 (200 mg, 0.404 mmol), compound 6-a (150 mg, 0.606 mmol) Pd(dppf)Cl₂ (33 mg, 0.040 mmol) and Na₂CO₃ (128 mg, 1.21 mmol) in dioxane (8 mL) and H₂O (2 mL) was degassed and purged with N₂ for 3 times, then stirred at 80° C. for 16 hours under N₂ atmosphere. The reaction mixture was concentrated and the residue was purified by flash column (SiO₂, 0% to 4% MeOH in DCM) to give 1-(1-(benzo[d][1,3]dioxol-5-yl)-3-tosyl-3,6,8,9-tetrahydro-7H-pyrrolo[2,3-c][2,7]naphthyridin-7-yl)ethan-1-one (200 mg, crude) as a yellow gum.

### Step 2. Synthesis of 1-(1-(benzo[d][1,3]dioxol-5yl)-3,6,8,9-tetrahydro-7H-pyrrolo[2, 3-c][2, 7]naphthyridin-7yl)ethan-1-one

A mixture of compound 6-b (200 mg, 0.409 mmol) and KOH (69 mg, 1.2 mmol) in THF (4 mL), MeOH (2 mL) and H₂O (1 mL) was stirred at 80° C. for 2 hours. The reaction mixture was filtered and the filtrate was concentrated. The residue was purified by prep-HPLC (0.04% o NH₃H₂O+10 mM NH₄HCO₃ as an additive) and lyophilized to give the title compound (7.0 mg, yield: 5% o) as a white solid.

¹H NMR (400 MHz, DMSO-d₆) δ 5 2.05-2.12 (3H, in), 2.71-2.90 (2H, in), 3.54-3.62 (2H, in), 4.66-4.78 (2H, in), 6.06 (2H, s), 6.84-7.04 (3H, in), 7.33-7.41 (1H, in), 8.09 (1H, s), 11.72 (1H, brs).

The following compounds were synthesized analogously to Example 6

| Example | | | |
|---|---|---|---|
| No. | Structure | Name | ¹H NMR (400 MHz) |
| 7 | 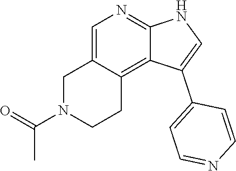 [SMILES: CC(=O)N1CCc2c(cnc3[nH]cc(-c4ccncc4)c23)C1] | 1-(1-(pyridin-4-yl)- 3,6,8,9-tetrahydro-7H- pyrrolo[2,3- c][2,7]naphthyridin-7- yl)ethan-1-one | DMSO-d₆; δ 2.06-2.16 (3H, m), 2.78-2.97 (2H, m), 3.54- 3.64 (2H, m), 4.67-4.82 (2H, m), 7.45-7.53 (2H, m), 7.62- 7.69 (1H, m), 8.15 (1H, s), 8.53-8.60 (2H, m), 12.07 (1H, brs). |
| 8 | 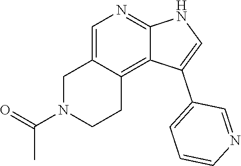 [SMILES: CC(=O)N1CCc2c(cnc3[nH]cc(-c4cccnc4)c23)C1] | 1-(1-(pyridin-3-yl)- 3,6,8,9-tetrahydro-7H- pyrrolo[2,3- c][2,7]naphthyridin-7- yl)ethan-1-one | DMSO-d₆; δ 2.05-2.14 (3H, m), 2.68-2.87 (2H, m), 3.54- 3.64 (2H, m), 4.67-4.82 (2H, m), 7.44 (1H, dd, J = 8.0, 4.8 Hz), 7.57 (1H, d, J = 4.0 Hz), 7.84-7.94 (1H, m), 8.13 (1H, s), 8.53 (1H, dd, J = 4.8, 1.6 Hz), 8.64-8.72 (1H, m), 11.96 (1H, brs). |
| 9 | 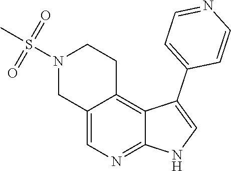 [SMILES: CS(=O)(=O)N1CCc2c(cnc3[nH]cc(-c4ccncc4)c23)C1] | 7-(methylsulfonyl)-1- (pyridine- 4-yl)-6,7,8,9- tetrahydro-3H- pyrrolo[2,3- c][2,7]naphthyridine | DMSO-d₆; δ 2.93-3.01 (5H, m), 3.35-3.36 (1H, m), 3.37- 3.39 (1H, m), 4.51 (2H, s), 7.46-7.56 (2H, m), 7.66 (1H, s), 8.16 (1H, s), 8.52-8.62 (2H, m), 12.10 (1H, brs). |
| 10 | 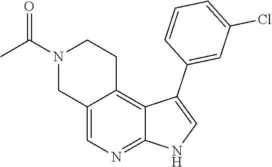 [SMILES: CC(=O)N1CCc2c(cnc3[nH]cc(-c4cccc(Cl)c4)c23)C1] | 1-(1-(3-chlorophenyl)- 3,6,8,9-tetrahydro-7H- pyrrolo[2,3- c][2,7]naphthyridin-7- yl)ethan- 1-one | DMSO-d₆; δ 2.05-2.14 (3H, m), 2.69-2.89 (2H, m), 3.55- 3.60 (2H, m), 4.63-4.83 (2H, m), 7.33-7.46 (3H, m), 7.47- 7.57 (2H, m), 8.12 (1H, s), 11.90 (1H, brs). |
| 12 | 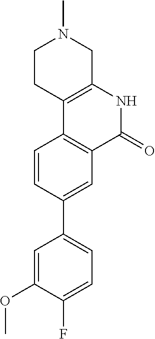 [SMILES: COc1cc(-c2ccc3c4c([nH]c(=O)c3c2)CN(C)CC4)ccc1F] | 8-(4-fluoro-3- methoxyphenyl)- 3-methyl-1,3,4,5- tetrahydrobenzo[c][1,7] naphthyridin-6(2H)- one | CDCl₃; δ 2.57 (3H, s), 2.87- 2.92 (3H, m), 3.54 (2H, s), 3.97-4.03 (4H, m), 7.17-7.25 (2H, m), 7.29-7.33 (1H, m), 7.67 (1H, d, J = 8.8 Hz), 7.93 (1H, dd, J = 8.4, 2.0 Hz), 8.62 (1H, d, J = 1.6 Hz), 10.16 (1H, brs). |
| 13 | 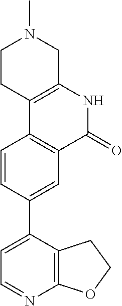 [SMILES: CN1CCc2c([nH]c(=O)c3cc(-c4ccnc5c4CCO5)ccc23)C1] | 8-(2,3-dihydrofuro[2,3- b] pyridin-4-yl)-3-methyl- 1,3,4,5- tetrahydrobenzo[c][1,7] naphthyridin-6(2H)- one | CDCl₃; δ 2.56 (3H, s), 2.85- 2.92 (4H, m), 3.46-3.53 (4H, m), 4.69 (2H, t, J = 8.8 Hz), 7.02 (1H, d, J = 5.6 Hz), 7.71 (1H, d, J = 8.8 Hz), 7.89 (1H, dd, J = 8.8, 2.0 Hz), 8.10-8.15 (2H, m), 8.61 (1H, d, J = 2.0 Hz). |

### Example 14

### 3-methyl-3H-pyrazolo[3,4-c]quinoline-7-carbonitrile

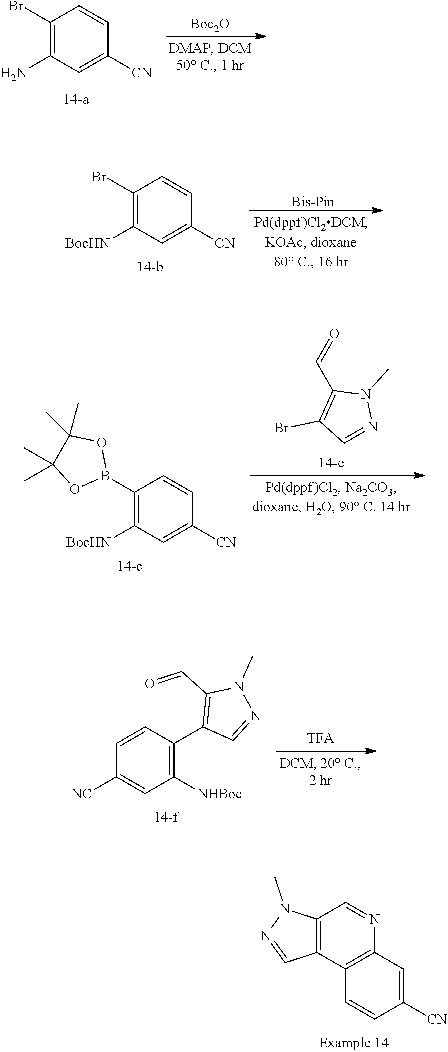

### Step 1. Synthesis of tert-butyl (2-bromo-5-cyanophenyl)carbamate

A mixture of compound 14-a (2.00 g, 10.2 mmol), Boc₂O (2.66 g, 12.2 mmol), DMAP (248 mg, 2.03 mmol) in DCM (20 mL) was stirred at 50° C. for 1 hour. The reaction mixture was concentrated and the residue was purified by flash silica gel column (PE/EtOAc=3/1) to afford tert-butyl (2-bromo-5-cyanophenyl)carbamate (1.00 g, yield: 33%) as a white solid.

### Step 2. Synthesis of tert-butyl (5-cyano-2-(4,4,5,5-tetramethyl-1, 3,2-dioxaborolan-2-yl)phenyl)carbamate

A mixture of compound 2 (370 mg, 1.25 mmol), Bis-Pin (379 mg, 1.49 mmol), KOAc (244 mg, 2.49 mmol), Pd(dppf)Cl₂·DCM (102 mg, 0.120 mmol) in dioxane (10 mL) was degassed and purged with N₂ for 3 times, then the mixture was stirred at 80° C. for 16 hours under N₂ atmosphere. The reaction mixture was filtered and the filtrate was concentrated. The residue was purified by flash silica gel column (PE/EtOAc=5/1) to afford tert-butyl (5-cyano-2-(4,4,5,5-tetramethyl-1,3,2-dioxaborolan-2-yl)phenyl)carbamate (203 mg, yield: 47%) as a white solid.

¹H NMR (400 MHz, DMSO-d₆) δ 1.27 (12H, s), 1.52 (9H, s), 7.46 (1H, d, J=8.0 Hz), 7.60 (1H, d, J=8.0 Hz), 7.66 (1H, s), 9.95 (1H, brs).

### Step 3. Synthesis of tert-butyl (5-cyano-2-(5-formyl-1-methyl-1H-pyrazol-4-yl)phenyl)carbamate

A mixture of compound 14-c (200 mg, 0.580 mmol), compound 14-e (132 mg, 0.697 mmol), Na₂CO₃ (123 mg, 1.16 mmol), Pd(dppf)Cl₂ (43 mg, 0.058 mmol) in dioxane (5 mL) and H₂O (1 mL) was degassed and purged with N₂ for 3 times, then the mixture was stirred at 90° C. for 14 hours under N₂ atmosphere. The reaction mixture was quenched by addition water (20 mL), then extracted with EtOAc (20 mL×2). The combined organic layer was dried over anhydrous Na₂SO₄, filtered and concentrated. The residue was purified by flash silica gel column (PE/EtOAc=1/1) to give tert-butyl (5-cyano-2-(5-formyl-1-methyl-1H-pyrazol-4-yl)phenyl)carbamate (138 mg, yield: 73%) as a white solid.

¹H NMR (400 MHz, DMSO-d₆) δ 1.52 (9H, s), 3.89 (3H, s), 6.91 (1H, d, J=6.8 Hz), 6.98 (1H, d, J=7.2 Hz), 7.61 (1H, dd, J=8.0, 1.6 Hz), 7.83 (1H, d, J=8.0 Hz), 8.02 (1H, s), 8.06 (1H, s).

### Step 4. Synthesis of 3-methyl-3H-pyrazolo[3,4-c]quinoline-7-carbonitrile

A solution of compound 14-f (60 mg, 0.18 mmol) in DCM (4 mL) and TFA (4 mL) was stirred at 20° C. for 2 hours. The reaction mixture was concentrated and the crude product was re-crystallized from HOAc/MeOH (2 mL, 1/1), then washed with CH₃CN (1 mL) to give the title compound (7.83 mg, yield: 20%) as a white solid.

¹H NMR (400 MHz, CDCl₃) δ 4.37 (3H, s), 7.87 (1H, dd, J=8.4, 1.6 Hz), 8.30 (1H, d, J=8.0 Hz), 8.48 (1H, s), 8.59 (1H, d, J=1.6 Hz), 9.28 (1H, s).

### Example 15

### 1-(9-(pyridin-4-yl)-1,3,4,7-tetrahydro-2H-pyrrolo[2,3-c][2,6]naphthyridin-2-yl)ethan-1-one

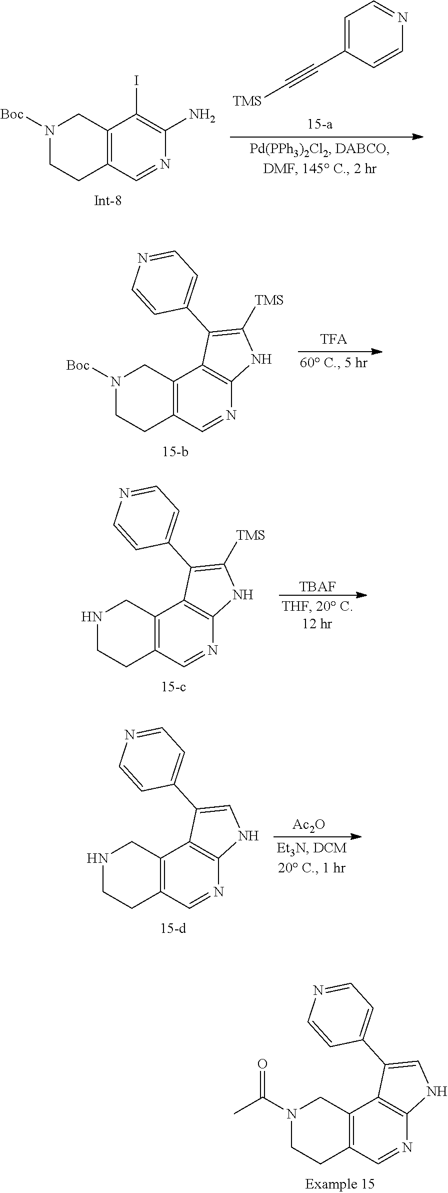

### Step 1. Synthesis of tert-butyl 9-(pyridin-4-yl)-8-(trimethylsilyl)-1,3,4, 7-tetrahydro-2H-pyrrolo[2,3-c][2,6]naphthyridine-2-carboxylate

A mixture of compound Int-8 (160 mg, 0.426 mmol), compound 15-a (224 mg, 1.28 mmol), DABCO (96 mg, 0.85 mmol) and Pd(PPh₃)₂Cl₂ (30 mg, 0.043 mmol) in anhydrous DMF (5 mL) was degassed and purged with N₂ for 3 times, then stirred at 145° C. for 2 hours under N₂ atmosphere. The mixture was concentrated and the residue was purified by Combi Flash (0% to 100% EtOAc in PE) to give tert-butyl 9-(pyridin-4-yl)-8-(trimethylsilyl)-1,3,4,7-tetrahydro-2H-pyrrolo[2,3-c][2,6]naphthyridine-2-carboxylate (80 mg, yield: 40%) as a light yellow solid.

### Step 2. Synthesis of 9-(pyridin-4-yl)-8-(trimethylsilyl)-2,3,4, 7-tetrahydro-1H-pyrrolo[2,3-c][2,6]naphthyridine

A solution of compound 15-b (80 mg, 0.19 mmol) in TFA (3 mL) was stirred at 60° C. for 5 hours. The reaction mixture was concentrated to give 9-(pyridin-4-yl)-8-(trimethylsilyl)-2,3,4,7-tetrahydro-1H-pyrrolo[2,3-c][2,6]naphthyridine (70 mg, crude) as a light yellow solid.

### Step 3. Synthesis of 9-(pyridin-4-yl)-2,3,4, 7-tetrahydro-1H-pyrrolo[2,3-c][2,6]naphthyridine

To a solution of compound 15-c (70 mg, 0.22 mmol) in anhydrous THF (3 mL) was added TBAF (0.43 mL, 0.43 mmol, 1N in THF) and stirred at 20° C. for 12 hours. The reaction mixture was quenched with saturated aqueous NH₄Cl (20 mL) at 20° C., then washed with EtOAc (10 mL×2). The aqueous layer was concentrated and the residue was suspended in DCM/MeOH (30 mL, 10/1) and filtered. The filtrate was concentrated to give 9-(pyridin-4-yl)-2,3,4,7-tetrahydro-1H-pyrrolo[2,3-c][2,6]naphthyridine (60 mg, crude) as a white solid.

### Step 4. Synthesis of 1-(9-(pyridin-4-yl)-1,3,4, 7-tetrahydro-2H-pyrrolo[2,3-c][2,6]naphthyridin-2-yl)ethan-1-one

To a suspension of compound 15-d(40 mg, 0.16 mmol) and Et₃N (32 mg, 0.32 mmol) in DCM (3 mL) was added acetic anhydride (44 mg, 0.43 mmol), then stirred at 20° C. for 1 hour. The reaction mixture was concentrated and the residue was purified by pre-HPLC (0.225% FA as an additive), then lyophilized to give the title compound (10.5 mg, yield: 15%) as a white solid.

¹H NMR (400 MHz, DMSO-d₆) δ 1.80-2.09 (3H, m), 2.93-3.01 (2H, m), 3.65-3.75 (2H, m), 4.65-4.70 (2H, m), 7.42-7.53 (2H, m), 7.56-7.57 (1H, d, J=1.6 Hz), 8.13 (1H, s), 8.52-8.63 (2H, m), 12.08 (1H, brs).

The following compound was synthesized analogously to Example 15

| Example | | | |
|---|---|---|---|
| No. | Structure | Name | ¹H NMR (400 MHz) |
| 16 | 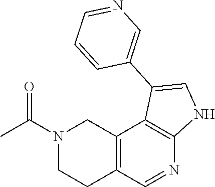 [SMILES: CC(=O)N1CCc2cnc3[nH]cc(-c4cccnc4)c3c2C1] | 1-(9-(pyridin-3-yl)- 1,3,4,7-tetrahydro- 2H-pyrrolo[2,3- c][2,6]naphthyridin- 2-yl)ethan-1-one | DMSO-d₆; δ 1.79 (1H, s), 2.03 (2H, s), 2.79-3.00 (2H, m), 3.62- 3.76 (2H, m), 4.49-4.63 (2H, m), 7.40-7.50 (1H, m), 7.51-7.61 (1H, m), 7.83-8.00 (1H, m), 8.06-8.12 (1H, m), 8.49-8.61 (1H, m), 8.64- 8.75 (1H, m), 11.95 (1H, brs). |

### Example 17

### 8-(2-(dimethylamino)ethoxy)-2-(pyridin-4-yl)pyrrolo[1,2-a]quinoxalin-4(5H)-one

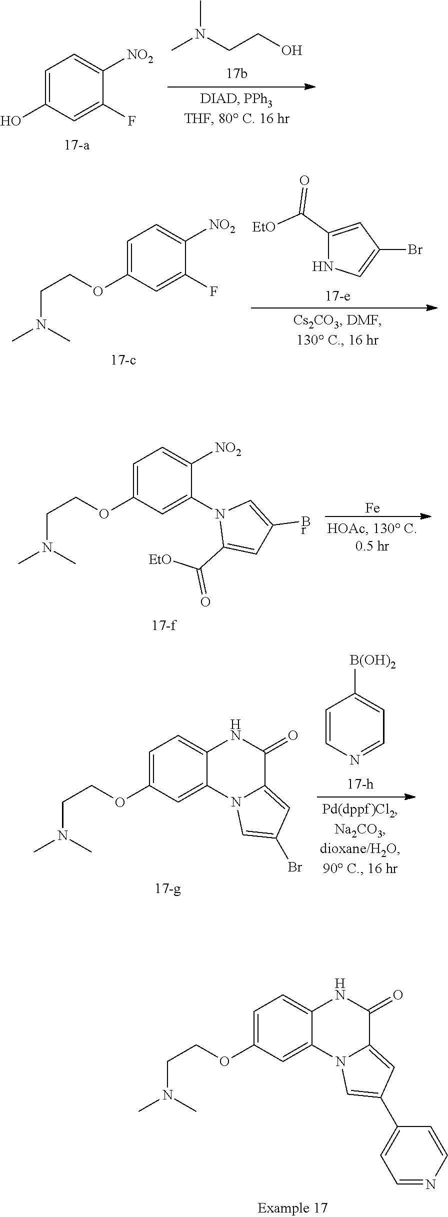

### Step 1. Synthesis of 2-(3-fluoro-4-nitrophenoxy)-N,N-dimethylethan-1-amine

The mixture of compound 17-a (2.00 g, 12.7 mmol), compound 17-b (1.82 g, 20.1 mmol) and PPh₃ (6.68 g, 25.5 mmol) in THF (30 mL) was degassed and purged with N₂ for 3 times, then added DIAD (4.12 g, 20.4 mmol) and the resulting mixture was stirred at 80° C. for 16 hours under N₂ atmosphere. The reaction mixture was cooled to 25° C. and 6N aqueous HCl was added to adjust pH=2, then diluted with H₂O (60 mL) and washed with EtOAc (60 mL×3).

The aqueous layer was basified with 2N aqueous NaOH to pH=10 and extracted with EtOAc (60 mL×3). The combined organic layers were dried over anhydrous Na₂SO₄, filtered and concentrated. The residue was purified by silica gel column (DCM/MeOH=10/1) to afford 2-(3-fluoro-4-nitrophenoxy)-N,N-dimethylethan-1-amine (920 mg, yield: 16%) as yellow oil.

¹H NMR (400 MHz, DMSO-d₆) δ 2.20 (6H, s), 2.64 (2H, t, J=6.0 Hz), 4.21 (2H, t, J=5.6 Hz), 6.91-7.03 (1H, m), 7.15-7.25 (1H, m), 8.13 (1H, t, J=9.2 Hz).

### Step 2. Synthesis of 2-(3-fluoro-4-nitrophenoxy)-N,N-dimethylethan-1-amine ethyl 4-bromo-1-(5-(2-(dimethylamino)ethoxy)-2-nitrophenyl)-1H-pyrrole-2-carboxylate

A mixture of compound 17-c (920 mg, 4.03 mmol), compound 17-e (1.05 g, 4.84 mmol) and Cs₂CO₃ (1.58 g, 4.84 mmol) in DMF (15 mL) was stirred at 130° C. for 16 hours. The reaction mixture was diluted with H₂O (60 mL) and extracted with EtOAc (60 mL×3). The combined organic layers were dried over anhydrous Na₂SO₄, filtered and concentrated. The residue was purified by silica gel column (DCM/MeOH=10/1) to afford ethyl 4-bromo-1-(5-(2-(dimethylamino)ethoxy)-2-nitrophenyl)-1H-pyrrole-2-carboxylate (620 mg, yield: 27%) as yellow gum.

¹H NMR (400 MHz, DMSO-d₆) δ 1.08 (3H, t, J=7.2 Hz), 2.21 (6H, s), 2.65 (2H, t, J=6.0 Hz), 4.03 (2H, q, J=7.2 Hz), 4.22 (2H, t, J=5.6 Hz), 7.08 (1H, d, J=2.0 Hz), 7.21 (1H, d, J=2.8 Hz), 7.51 (1H, d, J=2.0 Hz), 7.95 (1H, s), 8.20 (1H, d, J=9.2 Hz).

### Step 3. Synthesis of 2-bromo-8-(2-(dimethylamino)ethoxy)pyrrolo[1,2-a]quinoxalin-4(5H)-one

To a solution of compound 17-f (120 mg, 0.282 mmol) in HOAc (1 mL) was added Fe (63 mg, 1.1 mmol) with stirring and then stirred at 130° C. for 0.5 hour. The reaction mixture was cooled to 25° C. and basified with 2N aqueous NaOH to pH=10 and filtered. The solid was washed with MeOH (20 mL×3) and the filtrate was concentrated. The residue was purified by silica gel column (DCM/MeOH=10/1) to afford 2-bromo-8-(2-(dimethylamino)ethoxy)pyrrolo[1,2-a]quinoxalin-4(5H)-one (100 mg, yield: 97%) as yellow oil.

### Step 4. Synthesis of 8-(2-(dimethylamino)ethoxy)-2-(pyridin-4-yl)pyrrolo[1,2-a]quinoxalin-4(5H)-one

A mixture of compound 17-g (100 mg, 0.286 mmol), 4-pyridylboronic acid (42 mg, 0.34 mmol), Na₂CO₃ (91 mg, 0.86 mmol) and Pd(dppf)Cl₂ (21 mg, 0.029 mmol) in 1, 4-dioxane (2 mL) and H₂O (0.4 mL) was degassed and purged with N₂ for 3 times and then stirred at 90° C. for 16 hours under N₂ atmosphere. The reaction mixture was concentrated and the residue was purified by silica gel column (DCM/MeOH=5/1), then further purified by prep-HPLC (Method C; 0.225S FA as an additive) and lyophilized to afford the title compound (24.81 mg, yield: 25D, FA salt) as a yellow solid.

¹H NMR (400 MHz, DMSO-d₆) δ 2.24-2.32 (6H, in), 2.73 (2H, t, J=5.6 Hz), 4.17 (2H, t, J 5.6 Hz), 6.97 (1H, dd, J=8.8, 2.4 Hz), 7.24 (1H, d, J 8.8 Hz), 7.60 (1H, d, J=1.6 Hz), 7.73 (1H, d, J=2.4 Hz), 7.75-7.80 (2H, i), 8.16 (1H, d, J=1.6 Hz), 8.55-8.60 (2H, n), 8.96 (1H, s), 11.20 (1H, brs).

The following compound was synthesized analogously to Example 17

| Example | | | |
|---|---|---|---|
| No. | Structure | Name | ¹H NMR (400 MHz) |
| 18 | 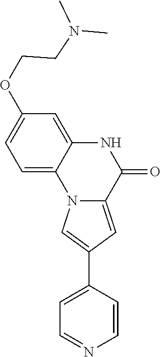 [SMILES: CN(C)CCOc1ccc2c(c1)[nH]c(=O)c1cc(-c3ccncc3)cn12] | 7-(2- (dimethylamino)ethoxy)- 2-(pyridin-4- yl)pyrrolo[1,2- a]quinoxalin-4(5H)-one | DMSO-d₆; δ 2.29 (6H, s), 2.68-2.79 (2H, m), 4.11 (2H, t, J = 5.6 Hz), 6.85 (1H, d, J = 2.8 Hz), 6.90 (1H, dd, J = 8.8, 2.4 Hz), 7.58 (1H, d, J = 1.2 Hz), 7.75-7.80 (2H, m), 8.03 (1H, d, J = 8.8 Hz), 8.10-8.20 (1H, m), 8.55-8.60 (2H, m), 8.85 (1H, s), 11.26 (1H, brs). |

Examples 19-26 were prepared analogously to the examples above.

| Example | |
|---|---|
| No. | Structure |
| 19 | 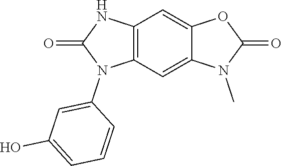 [SMILES: Cn1c(=O)oc2cc3[nH]c(=O)n(-c4cccc(O)c4)c3cc21] |
| 20 | 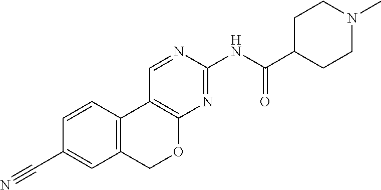 [SMILES: CN1CCC(C(=O)Nc2ncc3c(n2)OCc2cc(C#N)ccc2-3)CC1] |
| 21 | 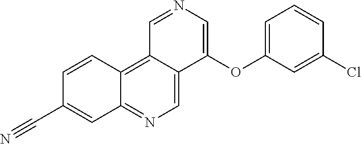 [SMILES: N#Cc1ccc2c(c1)ncc1c(Oc3cccc(Cl)c3)cncc12] |
| 22 | 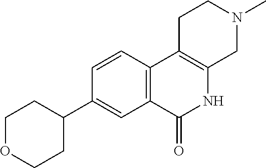 [SMILES: CN1CCc2c([nH]c(=O)c3cc(C4CCOCC4)ccc23)C1] |
| 23 | 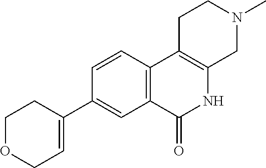 [SMILES: CN1CCc2c([nH]c(=O)c3cc(C4=CCOCC4)ccc23)C1] |
| 24 | 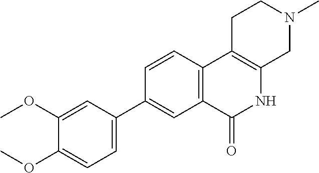 |
| 25 | 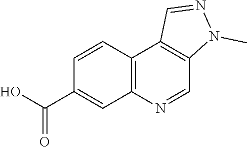 [SMILES: Cn1ncc2c3ccc(C(=O)O)cc3ncc21] |
| 26 | 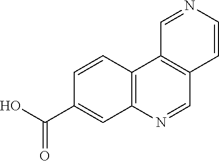 [SMILES: O=C(O)c1ccc2c(c1)ncc1ccncc12] |

Compounds of Formula (I), (II), (III), and (IV) and related analogs and their associated LC-MS data are shown in the Table below. These compounds were prepared according to procedures analogous to the procedures above, with modifications where appropriate that are within the purview of one skilled in the art. Liquid Chromatography-Mass Spectrometry (LCMS) was taken on a quadruple Mass Spectrometer on Waters QDa/Acquity I-Class LCMS (Column: Phenomenex Kinetex EVO C18 (1.0×50 mm, 1.7 um)) operating in ESI (+) ionization mode. Flow Rate: 0.4 mL/min, Acquire Time: 1.5 min, Wavelength: UV215 & 254, Oven Temp.: 55° C.

| | | LC-MS | |
|---|---|---|---|
| | | Retention | |
| Cmpd. | | Time | m/z |
| No. | Compound structure | (min) | (M + H) |
| A1 | 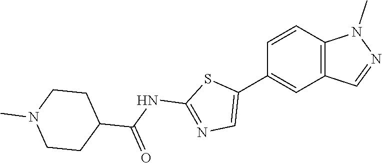 [SMILES: CN1CCC(C(=O)Nc2ncc(-c3ccc4c(cnn4C)c3)s2)CC1] | 0.448 | 356.2 |
| A2 | 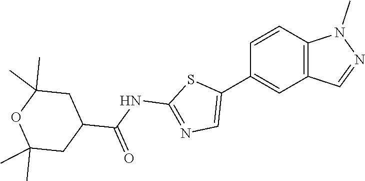 [SMILES: Cn1ncc2cc(-c3cnc(NC(=O)C4CC(C)(C)OC(C)(C)C4)s3)ccc21] | 0.768 | 399.2 |
| A3 | 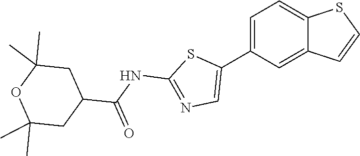 [SMILES: CC1(C)CC(C(=O)Nc2ncc(-c3ccc4sccc4c3)s2)CC(C)(C)O1] | 0.989 | 401.2 |
| A4 | 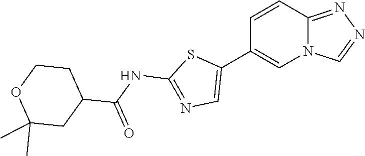 [SMILES: CC1(C)CC(C(=O)Nc2ncc(-c3ccc4nncn4c3)s2)CCO1] | 0.507 | 358.2 |
| A5 | 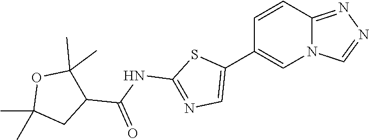 [SMILES: CC1(C)CC(C(=O)Nc2ncc(-c3ccc4nncn4c3)s2)C(C)(C)O1] | 0.555 | 372.2 |

### DYRK1A Inhibition Assay

### Materials

### DYRK1A—Invitrogen #PV4105

Ser/Thr 18 peptide—Invitrogen #PR8227U
Phos-Ser/Thr 18 peptide—Invitrogen #PR8229U
Assay plate—PerkinElmer #6007279
ATP—Sigma #A7699-5G
Development reagent A—Invitrogen #PR5194B
Development buffer—Invitrogen #PR4876B
   Compound preparation
1. Test compounds were diluted to 1 mM in DMSO
2. Stock was further diluted 3-fold using Echo platform
3. 100 nL of DMSO into Columns 1 and 24 and 100 nL of compounds dilutions to Columns 2-23 in a plate.

### Assay Procedure

1. Add 5 μL enzyme & substrate mixture to each well in Column 2-23 and A24-H24 wells of the 384-well assay plate;
2. Add 5 μL 0% Phosphorylation control to A1-H1 and I₂₄-P24 wells of the assay plate;
3. Add 5 μL 100% Phosphorylation control to I1-P1 wells of the assay plate;
4. Spin the assay plate (1000 rpm, 1 minute @23° C.);
5. Incubate enzyme with compounds for 15 minutes at 23° C.;
6. Add 5 μL ATP solution to each well of the assay plate;
7. Spin the plate (1000 rpm, 1 minute @23° C.);
8. Incubate the assay plate for 90 minutes at 23° C.;
9. Add 10 μL Development reagent A to each well of the assay plate;
10. Centrifuge the plate at 1000 rpm about 15 seconds and seal a film over assay plate. Incubate the assay plate for 30 minutes at 23° C.
11. Read assay plates on Envision (see Tables A-D).

### Final Compound Concentrations

Assay buffer: 50 mM Hepes pH7.5, 10 mM MgCl₂, 1 mM EDTA, 0.01% Brij-35
DYRK1A: 1 nM
ATP: 20 μM
Ser/Thr 18 peptide: 2 μM
Reaction time: 90 minutes

TABLE A
DYRK1 Inhibition by Selected Compounds of Formula (I)

| Compound | DYRK1 |
|---|---|
| Number | IC₅₀ |
| 1 | A |
| 2 | B |
| 3 | B |
| 4 | D |
| 5 | B |
| 6 | A |
| 7 | B |
| 8 | A |
| 9 | A |
| 10 | C |
| 11 | C |
| 12 | B |
| 13 | A |
| 14 | A |
| 15 | |
| 16 | B |
| 17 | A |
| 18 | A |
| 19 | A |
| 20 | A |
| 21 | B |
| 22 | C |
| 23 | A |
| 24 | A |
| 25 | A |
| 26 | A |
| 27 | A |
| 28 | A |
| 29 | A |
| 30 | C |
| 31 | C |
| 32 | C |
| 33 | C |
| 34 | C |
| 35 | C |
| 36 | C |
| 37 | B |
| 38 | B |
| 39 | A |
| 40 | A |
| 41 | A |
| 42 | A |
| 43 | B |
| 44 | C |
| 45 | N/A |
| 46 | B |
| 47 | A |
| 48 | N/A |
| 49 | B |
| 50 | B |
| 51 | A |
| 52 | C |
| 53 | C |
| 54 | A |
| 55 | A |
| 56 | A |
| 57 | D |
| 58 | B |
| 59 | A |
| 60 | B |
| 61 | A |
| 62 | B |
| 63 | A |
| 64 | B |
| 65 | |
| 66 | A |
| 67 | A |
| 68 | |
| 69 | B |
| 70 | B |
| 71 | C |
| 72 | A |
| 73 | B |
| 74 | B |
| 75 | B |
| 76 | B |
| 77 | A |
| 78 | A |
| 79 | B |
| 80 | C |
| 81 | C |
| 82 | C |
| 83 | C |
| 84 | D |
| 85 | B |
| 86 | B |
| 87 | B |
| 88 | A |
| 89 | B |
| 90 | N/A |
| 91 | N/A |
| 92 | N/A |
| 93 | N/A |
| 94 | N/A |
| 95 | C |
| 96 | D |
| 97 | N/A |

(A ≤ 10 nM; 10 nM < B ≤ 100 nM; 100 nM < C ≤ 1 μM; D > 1 μM)

TABLE B
DYRK1 Inhibition by Selected Compounds of Formula (II)

| Compound | |
|---|---|
| Number | DYRK1 IC₅₀ |
| 1 | A |
| 2 | A |
| 3 | C |
| 4 | C |
| 5 | B |
| 6 | B |
| 7 | B |
| 8 | ND |
| 9 | B |

(A ≤ 10 nM; 10 nM < B ≤ 100 nM; 100 nM < C ≤ 1 μM; D > 1 μM)
ND = not determined

TABLE C
DYRK1 Inhibition by Selected Compounds of Formula (III)

| Compound | DYRK1 IC₅₀ |
|---|---|
| Number | (nM) |
| 1 | B |
| 2 | A |
| 3 | B |
| 4 | A |
| 5 | C |
| 6 | C |
| 7 | B |
| 8 | B |
| 9 | B |
| 10 | B |

(A ≤ 50 nM; 50 nM < B ≤ 200 nM; 200 nM < C ≤ 1 μM; D > 1 μM)

TABLE D
DYRK1 Inhibition by Selected Compounds of Formula (IV)

| Compound | DYRK1 IC₅₀ |
|---|---|
| Number | (nM) |
| 1 | A |
| 2 | C |
| 3 | C |
| 4 | C |
| 5 | B |
| 6 | A |
| 7 | B |
| 8 | C |
| 9 | C |
| 10 | C |
| 11 | C |
| 12 | C |
| 13 | C |
| 14 | A |
| 15 | C |
| 16 | C |
| 17 | A |
| 18 | A |
| 19 | C |
| 20 | C |
| 21 | D |
| 22 | D |
| 23 | D |
| 24 | D |
| 25 | D |
| 26 | D |

(A ≤ 500 nM; 500 nM < B ≤ 1 μM; 1 μM < C ≤ 5 μM; D > 5 μM)

What is claimed is:

1. A compound of Formula (I): 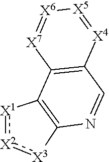
or a pharmaceutically acceptable salt thereof, wherein:
each dashed line represents a single bond or a double bond;
X¹ is CR^{1A}R^{1B}, NR^{A}, S, or O;
X² is CR^{2A}R^{2B}, C(═O), or NR^{B};
X³ is CR^{3A}R^{3B}, NR^{C}, S, or O;
X⁴ is CR⁴ or N;
X⁵ is CR⁵ or N;
X⁶ is CR⁶ or N;
X⁷ is CH, CF, or N;
R^{1A} is hydrogen, C1-C6 alkyl, —C(═O)NR^{F}R^{G}, —(C0-C6 alkyl)-5-6 membered heteroaryl,
—C(═O)-3-6 membered heterocyclyl optionally substituted with C1-C6 alkyl, a phenyl optionally substituted with halogen or —CO₂H, a 3-6 membered heterocyclyl optionally substituted with C1-C6 alkyl or —S(O₂)—C1-C6 alkyl, or a C3-C6 cycloalkyl optionally substituted with hydroxyl, —C(═O)NR^{F}R^{G}, —NR^{F}R^{G}, —CO₂R^{H}, or C1-C6 alkoxy;
R^{1B} is hydrogen or absent, wherein R^{1B} is absent when x¹ [symbol] x² is a double bond;
R^{2A} is hydrogen, C1-C6 alkyl, C1-C6 haloalkyl, C3-C6 cycloalkyl, 5-6 membered heteroaryl, a phenyl optionally substituted with —CO₂H, or a 3-6 membered heterocyclyl optionally substituted with C1-C6 alkyl, C1-C6 haloalkyl, C1-C6 hydroxyalkyl, or —(C0-C6 alkyl)-3-6 membered cycloalkyl optionally substituted with —CO₂H;
R^{2B} is hydrogen or absent, wherein R^{2B} is absent when either of x¹ [symbol] x² or x² [symbol] x³ is a double bond;
R^{3A} is hydrogen;
R^{3B} is hydrogen, C1-C6 alkyl, or absent, wherein R^{3B} is absent when x² [symbol] x³ is a double bond;
R⁴ is hydrogen, halogen, C1-C6 alkyl, or 3-6 membered heterocyclyl optionally substituted with C1-C6 alkyl;
R⁵ is hydrogen, —CO₂H, —C(═O)OCH₃, C1-C6 alkyl optionally substituted with hydroxyl, or a 5-6 membered heteroaryl optionally substituted with C1-C6 alkyl;
R⁶ is hydrogen or a 5-6 membered heteroaryl optionally substituted with 1-2 substituents independently selected from R^{D}, C1-C6 alkyl, and 4-6 membered heterocyclyl optionally substituted with hydroxyl;
R^{A} and R^{B} are independently absent, hydrogen, C1-C6 alkyl optionally substituted with 3-6 membered heterocyclyl or 5-6 membered heteroaryl, or a C3-C6 cycloalkyl optionally substituted with hydroxyl or C1-C6 alkoxy;
R^{C} is absent, hydrogen, or methyl;
R^{D} is 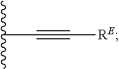
R^{E} is a C1-C6 alkyl, phenyl, 5-6 membered heteroaryl, or 4-6 membered heterocyclyl; wherein the C1-C6 alkyl, phenyl, 5-6 membered heteroaryl, and 4-6 membered heterocyclyl are each optionally substituted with 1-2 independently selected R¹;
each R^{F} and R^{G} are independently selected from hydrogen and C1-C6 alkyl; or R^{F} and R^{G} together with the nitrogen atom to which they are attached form a 4-6 membered heterocyclyl optionally substituted with C1-C6 alkyl;
R^{H} is hydrogen or C1-C6 alkyl;
each R^{I} is independently C1-C6 alkyl, C1-C6 alkoxy, halogen, hydroxyl, cyano, or trifluoromethyl.

2. The compound of claim 1, wherein X¹ is CR^{1A}R^{1B}, X² is CR^{2A}R^{2B}, X³ is NR^{C}, x¹ [symbol] x² is a double bond, X² [symbol] ³ is a single bond, R^{1B} is absent, and R^{2B} is absent.

3. The compound of claim 1, wherein X¹ is NR^{A}, X² is CR^{2A}R^{2B}, X³ is NR^{C}, x¹ [symbol] x² is a single bond, x² [symbol] x³ is a double bond, and R^{2B} and R^{C} are absent.

4. The compound of claim 1, wherein X¹ is NR^{A}, X² is CR^{2A}R^{2B}, X³ is NR^{C}, x¹ [symbol] x² is a double bond, X² [symbol] x³ is a single bond, and R^{2B} and R^{A} is absent.

5. The compound of claim 1, wherein X¹ is S, X² is CR^{2A}R^{2B}, X³ is CR^{3A}R^{3B}x¹ [symbol] x² is a single bond, x² [symbol] x³ is a double bond, R^{2B} is absent, and R^{3B} is absent.

6. The compound of claim 1, wherein X¹ is S, X² is CR^{2A}R^{2B}, X³ is NR^{C}, x¹ [symbol] x² is a single bond, x² [symbol] x³ is a double bond, and R^{2B} is absent.

7. The compound of claim 1, wherein X¹ is NR^{A}, X² is CR^{2A}R^{2B}, X³ is NR^{C}, x¹ [symbol] x² is a single bond, x² [symbol] x³ is a double bond, and R^{2B} and R^{C} are absent.

8. The compound of claim 1, wherein X¹ is CR^{1A}R^{1B}, X² is NR^{B}, X³ is NR^{C}, x¹ [symbol] x² is a single bond, and x² [symbol] x³ is a single bond, and R^{B} is absent.

9. The compound of claim 1, wherein X¹ is CR^{1A}R^{1B}, X² is C═O, X³ is NR^{C}, x¹ [symbol] x² is a single bond, x² [symbol] x³ is a single bond.

10. The compound of claim 1, wherein X¹ is CR^{1A}R^{1B}, X² is CR^{2A}R^{2B}, X³ is O, x¹ [symbol] x² is a single bond, x² [symbol] x³ is a single bond.

11. The compound of claim 1, wherein X¹ is CR^{1A}R^{1B}, X² is CR^{2A}R^{2B}, X³ is O, x¹ [symbol] x² is a double bond, x¹ [symbol] x² is a single bond, R^{1B} is absent, and R^{2B} is absent.

12. The compound of any one of claims 1-11, wherein X⁴ is CR⁴.

13. The compound of any one of claims 1-11, wherein X⁴ is N.

14. The compound of any one of claims 1-13, wherein X⁵ is CR⁵.

15. The compound of any one of claims 1-11, wherein X⁵ is N.

16. The compound of any one of claims 1-15, wherein X⁶ is CR⁶.

17. The compound of any one of claims 1-11, wherein X⁶ is N.

18. The compound of any one of claims 1-17, wherein X⁷ is CH.

19. The compound of any one of claims 1-17, wherein X⁷ is CF.

20. The compound of any one of claims 1-11, wherein X⁷ is N.

21. The compound of any one of claims 1-11, wherein one of X⁴, X⁵, X⁶, and X⁷ are N.

22. The compound of any one of claims 1-11, wherein two of X⁴, X⁵, X⁶, and X⁷ are N.

23. The compound of any one of claims 1-11, wherein X⁴ is CR⁴; X⁵ is CR⁵; X⁶ is CR⁶; and X⁷ is CH.

24. A compound of Formula (II): 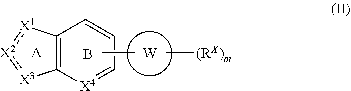
or a pharmaceutically acceptable salt thereof, wherein:
each dashed line represents a single bond or a double bond;
Rings A and B are aromatic;
Ring W is a 9 membered heteroaryl, a 9 membered heterocyclyl, or a 9 membered cycloalkyl;
each R^{X} is independently selected from C1-C6 alkyl, 3-6 membered heterocyclyl optionally substituted with C1-C6 alkyl, and C3-C6 cycloalkyl;
X¹ is CH, S, N, or NR^{A};
X² is N, CH, or CR²;
X³ is N, NR^{B}, O, CR³, or CH;
X⁴ is CH or N;
R² is benzyl or 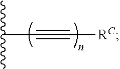
R³ is C1-C6 alkyl;
R^{A} is C1-C6 alkyl or C3-C6 cycloalkyl;
R^{B} is hydrogen or C3-C6 cycloalkyl;
R^{C} is 3-6 membered heterocyclyl optionally substituted with C1-C6 alkyl;
m is 0, 1, or 2; and
n is 0 or 1.

25. The compound of claim 24, wherein the dashed line between X¹ and X² represents a single bond and the dashed line between X² and X³ represents a double bond.

26. The compound of claim 24, wherein the dashed line between X¹ and X² represents a double bond and the dashed line between X² and X³ represents a single bond.

27. The compound of any one of claims 24-26, wherein Ring W is a 9 membered heteroaryl.

28. The compound of claim 27, wherein Ring W is pyrazolo[1,5-a]pyridinyl, pyrazolo[1,5-a]pyrimidinyl, 1H-pyrrolo[2,3-b]pyridinyl, 1H-pyrrolo[2,3-c]pyridinyl, thiazolo[4,5-c]pyridinyl, 1,3-dihydro-2H-imidazo[4,5-b]pyridin-2-only, indazolyl, or imidazo[1,2-a]pyrazine.

29. The compound of any one of claims 27-28, wherein Ring W is

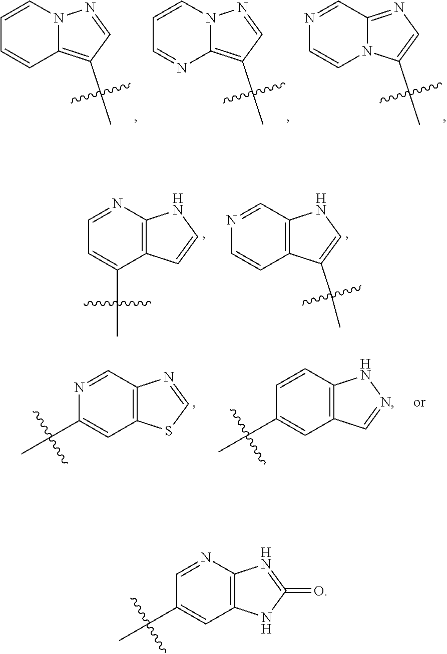

30. The compound of any one of claims 24-26, wherein Ring W is a 9 membered heterocyclyl.

31. The compound of claim 30, wherein Ring W is methylenedioxyphenyl.

32. The compound of claim 31, wherein Ring W is

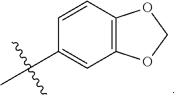 [SMILES: *c1ccc2c(c1)OCO2]

33. The compound of any one of claims 24-26, wherein Ring W is a 9 membered cycloalkyl.

34. The compound of any one of claims 24-33, wherein m is 1.

35. The compound of any one of claims 24-33, wherein m is 2.

36. A compound of Formula (III): 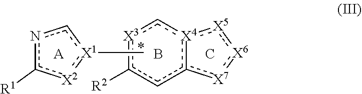
or a pharmaceutically acceptable salt thereof, wherein:
each dashed line represents a single bond or a double bond;
Rings A and B are aromatic;
Ring C is aromatic or partially saturated;
X¹ is C or N;
X² is CH, O, or S;
X³ is CH or N;
X⁴ is C or N;
X⁵ is CH, C(═O), O, S, NH, or NR^{A};
X⁶ is CH, N, or O;
X⁷ is CH, CR⁷, CH₂, CR^{B}R^{C}, or N;
R¹ is hydrogen or —XR^{D};
R² is hydrogen, —NHC(═O)-3-6 membered heterocyclyl optionally substituted with C1-C6 haloalkyl, or joins with the bond denoted with *;
R⁷ is C1-C6 alkyl optionally substituted with a 3-6 membered heterocyclyl optionally substituted with C(═O)OH;
R^{A} is -(3-6 membered heterocyclyl)ₘ-(C1-C6 alkyl)ₙ;
R^{B} and R^{C} are independently hydrogen or C1-C6 alkyl;
X is ethynylene, —NHC(═O)—, or —NHC(═O)OCH₂—;
R^{D} is 3-6 membered heterocyclyl optionally substituted with C1-C6 alkyl, 5-6 membered heteroaryl optionally substituted with C1-C6 alkyl, or C6-C10 aryl optionally substituted with C1-C6 alkyl;
m is 0 or 1; and
n is 0, 1, or 2;
provided that when n is 2, m is 1.

37. The compound of claim 36, wherein R¹ is hydrogen.

38. The compound of claim 36, wherein R¹ is —XR^{D}.

39. The compound of any one of claims 36-38, wherein X is ethynylene.

40. The compound of any one of claims 36-38, wherein X is —NHC(═O)—.

41. The compound of any one of claims 36-38, wherein X is —NHC(═O)OCH₂—.

42. A compound of Formula (IV): 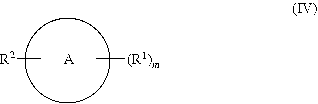
or a pharmaceutically acceptable salt thereof, wherein:
Ring A is a 12-14 membered fused tricyclic heterocyclyl comprising 2-5 nitrogen atoms or a 12-14 membered fused tricyclic heteroaryl comprising 2-5 nitrogen atoms;
R¹ is cyano, C1-C6 alkyl, —NHC(═O)(C1-C6 alkylene)ₙR^{A}, -Q-phenyl optionally substituted with 1-2 substituents independently selected from halogen, hydroxyl, and C1-C6 alkoxy; C3-C6 cycloalkyl optionally substituted with hydroxyl, —(C1-C6 alkylene)ₚ-5-10 membered heteroaryl, or 5-10 membered heterocyclyl;
R² is hydrogen, cyano, C1-C6 alkyl, —C(═O)—C1-C6 alkyl, —(SO₂)C1-C6 alkyl, —CO₂R^{B}, C1-C6 alkoxy optionally substituted with —NR^{C}R^{D};
R^{A} is 4-6 membered heterocyclyl optionally substituted with C1-C6 alkyl, or 5-10 membered heteroaryl optionally substituted with C1-C6 alkoxy or C1-C6 alkyl;
R^{B}, R^{C}, and R^{D} are independently hydrogen or C1-C6 alkyl;
Q is a bond or O;
m is 0 or 1;
n is 0 or 1; and
p is 0 or 1.

43. The compound of claim 42, wherein Ring A is 6H-isochromeno[3,4-d]pyrimidine, 5,7-dihydro-2H-imidazo[4′,5′:4,5]benzo[1,2-d]oxazole-2,6(3H)-dione, 5,7-dihydroimidazo[4,5-f]indazol-6(1H)-one, oxazolo[4,5-g]isoquinolin-2(1H)-one; 1,7-dihydro-6H-oxazolo[5,4-f]indazol-6-one, 6,7,8,9-tetrahydro-3H-pyrrolo[2,3-c][2,7]naphthyridine, benzo[c][2,6]naphthyridine, 1,3,4,5-tetrahydrobenzo[c][1,7]naphthyridin-6(2H)-one, 3H-pyrazolo[3,4-c]quinolone, 2,3,4,7-tetrahydro-1H-pyrrolo[2,3-c][2,6]naphthyridine, or pyrrolo[1,2-a]quinoxalin-4(5H)-one.

44. The compound of claim 42 or 43, wherein Ring A is

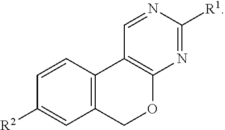

45. The compound of claim 42 or 43, wherein Ring A is

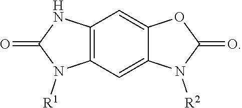

46. The compound of claim 42 or 43, wherein Ring A is

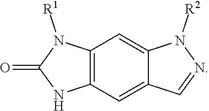

47. The compound of claim 42 or 43, wherein Ring A is

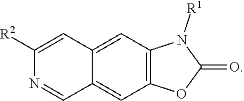

48. The compound of claim 42 or 43, wherein Ring A is

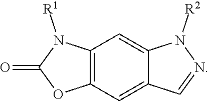

49. The compound of claim 42 or 43, wherein Ring A is

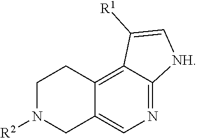

50. The compound of claim 42 or 43, wherein Ring A is

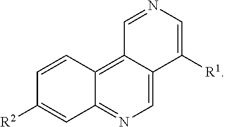

51. The compound of claim 42 or 43, wherein Ring A is

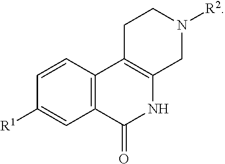

52. The compound of claim 42 or 43, wherein Ring A is

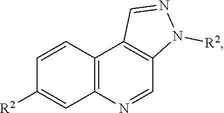

53. The compound of claim 42 or 43, wherein Ring A is

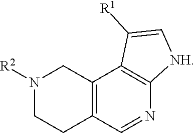

54. The compound of claim 42 or 43, wherein Ring A is H

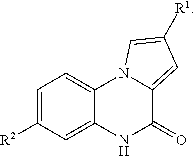

55. The compound of claim 1, wherein the compound is selected from a compound in Table 1, Table 2, Table 3, or Table 4, or a pharmaceutically acceptable salt of any of the foregoing.

56. A pharmaceutical composition comprising a compound of any one of claims 1-55, or a pharmaceutically acceptable salt thereof, and pharmaceutically acceptable diluent or carrier.

57. A method for treating a neurological disorder in a subject in need thereof, the method comprising administering to the subject a therapeutically effective amount of a compound of any one of claims 1-54, or a pharmaceutically acceptable salt thereof, or a pharmaceutical composition according to claim 55.

58. The method of claim 57, wherein the neurological disorder is selected from the group consisting of Down Syndrome, Alzheimer's disease, and Alzheimer's disease associated with Down Syndrome.

59. The method of claim 57 or 58, wherein the neurological disorder is selected Alzheimer's disease associated with Down syndrome.

60. A method of treating a DYRK1A-associated neurological disorder in a subject, the method comprising administering to a subject identified or diagnosed as having a DYRK1A-associated neurological disorder a therapeutically effective amount of a compound of any one of claims 1-54, or a pharmaceutically acceptable salt thereof, or a pharmaceutical composition according to claim 55.

61. A method for modulating DYRK1A in a mammalian cell, the method comprising contacting the mammalian cell with a therapeutically effective amount of a compound of any one of claims 1-54, or a pharmaceutically acceptable salt thereof.
